(19) United States
(12) Patent Application Publication
(10) Pub. No.: US 2026/0365647 A1
(43) Pub. Date: Nov. 19, 2026

(54) AKT1 MODULATORS

(71) Applicant: Alterome Therapeutics, Inc., San Diego, CA (US)

(72) Inventors: Michael David BARTBERGER, Sherman Oaks, CA (US); Yi FAN, San Diego, CA (US); Eric Anthony MURPHY, San Marcos, CA (US); Xuefeng ZHU, San Diego, CA (US); Elena V. DNEPROVSKAIA, San Diego, CA (US)

(21) Appl. No.: 19/106,010

(22) PCT Filed: Sep. 15, 2023
(86) PCT No.: PCT/US2023/032870

§ 371 Date: Feb. 24, 2025

Related U.S. Application Data
(60) Provisional application No. 63/376,252, filed on Sep. 19, 2022.

(51) Int. Cl. C07D 471/04; A61K 31/444; A61K 31/4545; A61K 31/496; A61K 31/5377; A61K 31/55; A61K 31/551; C07D 519/00

(52) U.S. Cl. CPC C07D 471/04; A61K 31/444; A61K 31/4545; A61K 31/496; A61K 31/5377; A61K 31/55; A61K 31/551; C07D 519/00

(57) ABSTRACT

Provided herein are inhibitors of AKT1, pharmaceutical compositions comprising the inhibitory compounds, and methods for using the AKT1 inhibitory compounds for the treatment of disease.

### CROSS REFERENCE

This application is a § 371 U.S. National Stage Entry of International Application No. PCT/US2023/032870, filed Sep. 15, 2023, which claims the benefit of U.S. Provisional Application No. 63/376,252, filed Sep. 19, 2022, all of which are incorporated herein by reference in their entirety.

### BACKGROUND

AKT is a protein kinase and mediates cell survival and proliferation by inhibiting pathways which promotes apoptosis. AKT signaling cascade dysfunction is observed in several cancer types and may be associated with tumor aggressiveness. Additionally, malfunction of AKT typically lead to enhanced proliferation, growth, survival, and resistance to apoptosis. Pharmaceutical agents with the ability to modulate AKT1 activity would be useful in the treatment of disease, such as cancer.

### BRIEF SUMMARY OF THE INVENTION

Provided herein are inhibitors of AKT1, pharmaceutical compositions comprising said inhibitory compounds, and methods for using said inhibitory compounds for the treatment of disease.

One embodiment provides a compound having the structure of Formula (I), or a pharmaceutically acceptable salt or solvate thereof:

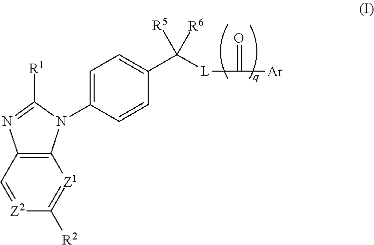

wherein:
Z¹ is N, C—H, or C—R³;
Z² is N, C—H, or C—R⁴;
Ar is selected from

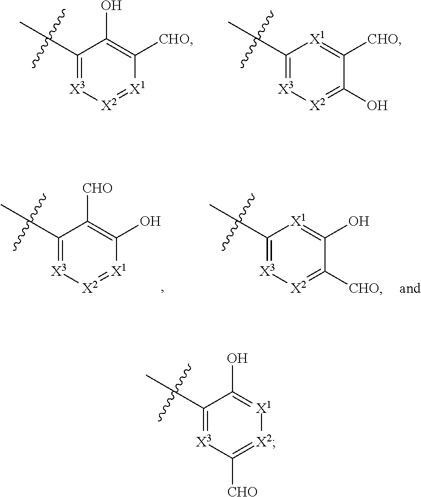

X¹ is N or C—R⁷;
X² is N or C—R⁷;
X³ is N or C—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, optionally substituted carbocyclyl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, halogen, —OH, —SH, optionally substituted C1-C6 alkoxy, —S-(optionally substituted C1-C6 alkyl), —CN, optionally substituted C1-C6 alkyl, optionally substituted amino, optionally substituted C1-C6 alkenyl, and optionally substituted aryl;
L is selected from —N(R⁸)—, —O—, or a divalent radical selected from:

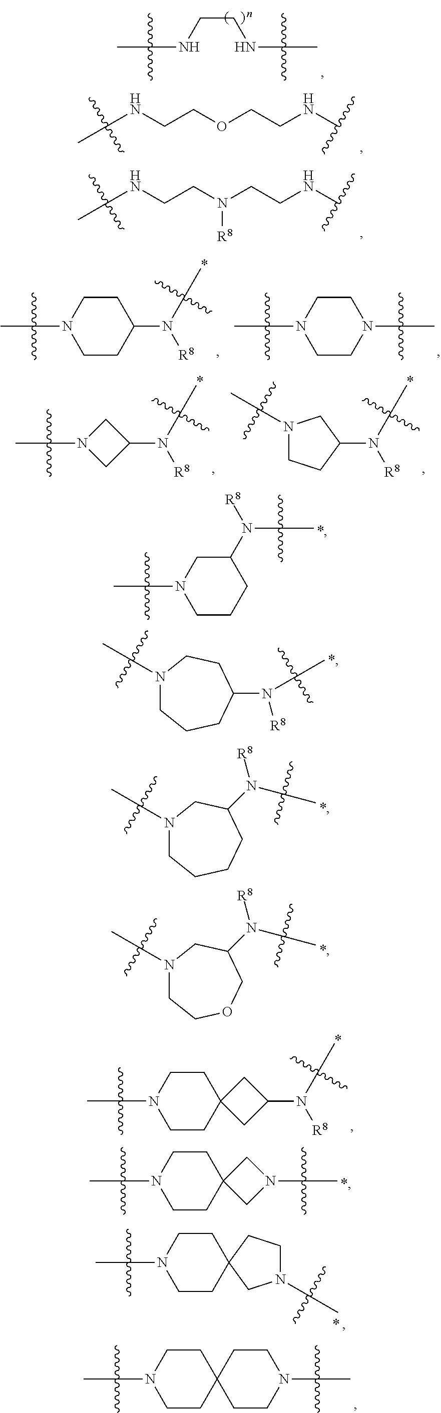

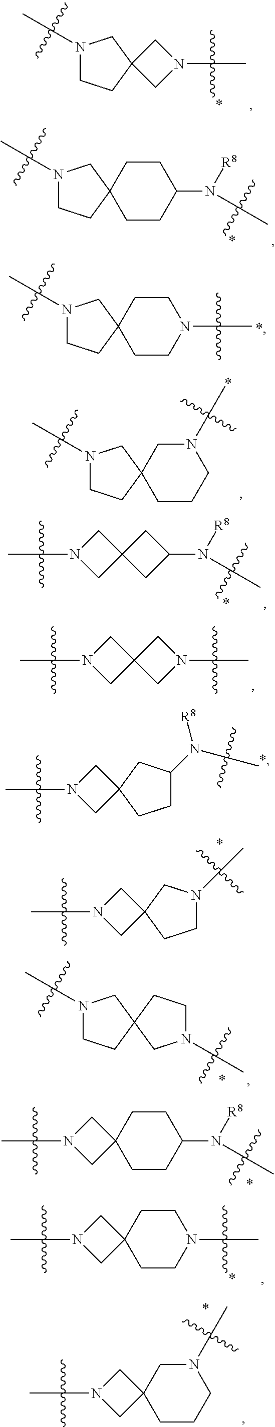

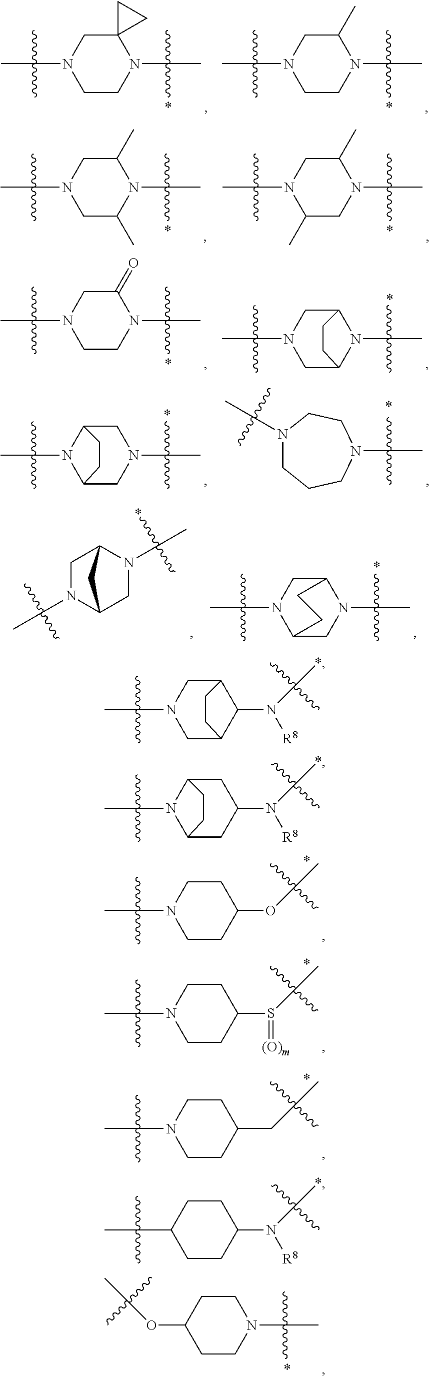

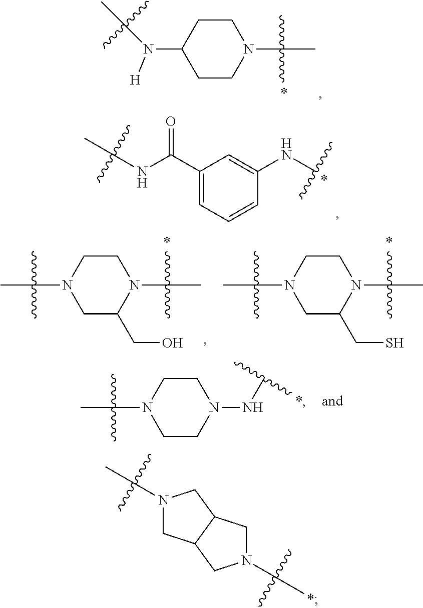

wherein the asterisk (*) indicates the bond to the —CO—Ar group;
R⁸ is hydrogen, optionally substituted C1-C6 alkyl, optionally substituted C3-C7 cycloalkyl, or optionally substituted heterocyclyl;
m is 0, 1, or 2;
n is 1, 2, or 3; and
q is 0 or 1.

One embodiment provides a compound having the structure of Formula (Ia), or a pharmaceutically acceptable salt or solvate thereof:

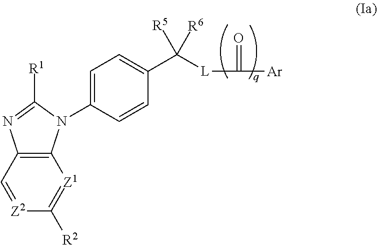

wherein:
Z¹ is N, C—H, or C—R³;
Z² is N, C—H, or C—R,
Ar is selected from

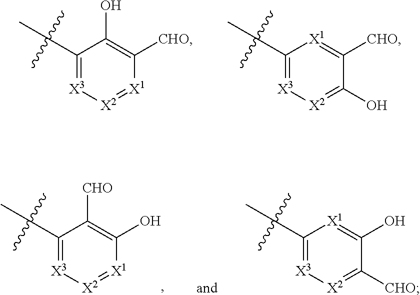

X¹ is N or C—R⁷;
X² is N or C—R⁷;
X³ is N or C—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R and R⁶ join together to form a carbocycle or heterocycle;
each R⁷ is independently selected from hydrogen, halogen, —OH, —SH, optionally substituted C1-C6 alkoxy, —S-(optionally substituted C1-C6 alkyl), —CN, optionally substituted C1-C6 alkyl, and optionally substituted aryl;
L is selected from —N(R⁸)-, or a divalent radical selected from:

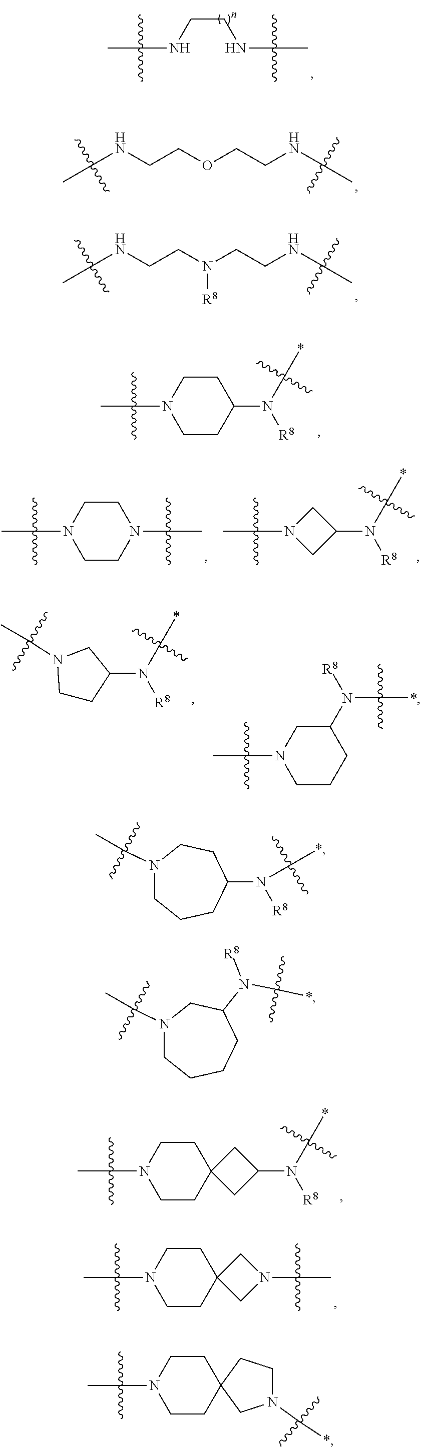

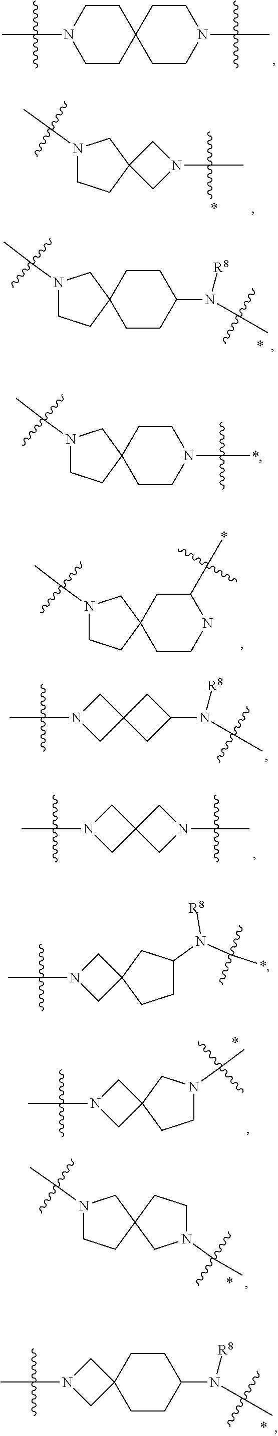

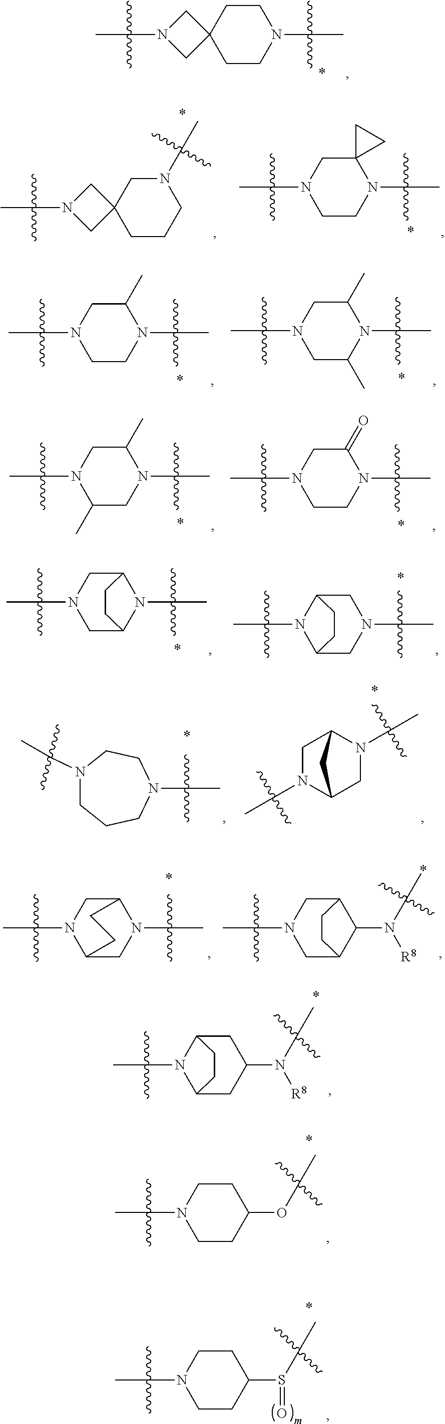

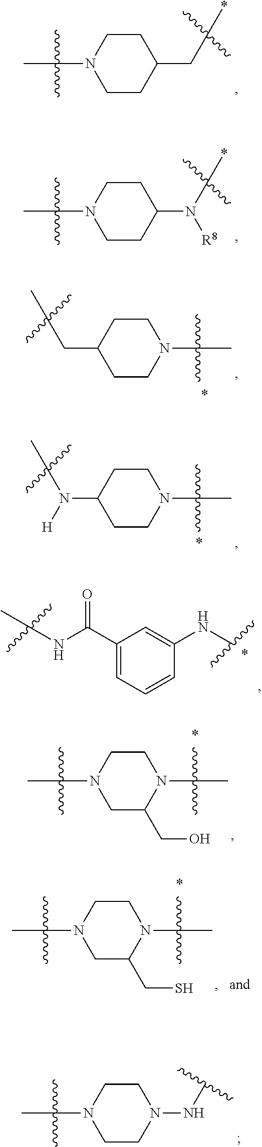

wherein the asterisk (*) indicates the bond to the —CO—Ar group;
R⁸ is hydrogen, optionally substituted C1-C6 alkyl, optionally substituted C3-C7 cycloalkyl, or optionally substituted heterocyclyl;
m is 0, 1, or 2;
n is 1, 2, or 3; and
q is 0 or 1.

One embodiment provides a compound having the structure of Formula (II), or a pharmaceutically acceptable salt or solvate thereof:

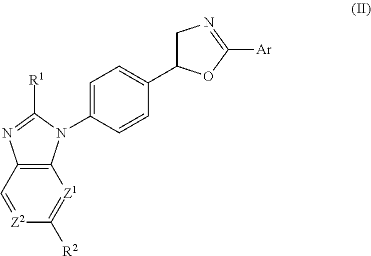

wherein:
Z¹ is N, C—H, or C—R³;
Z² is N, C—H, or C—R;
Ar is selected from

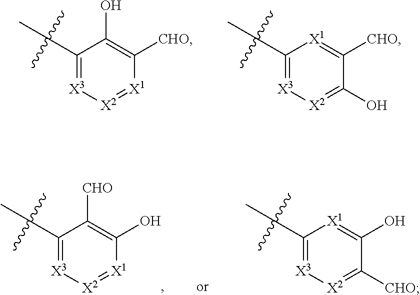

X¹ is N or C—R⁵;
X² is N or C—R⁵;
X³ is N or C—R⁵;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl; and
each R⁵ is independently selected from hydrogen, halogen, —OH, —SH, optionally substituted C1-C6 alkoxy, —S-(optionally substituted C1-C6 alkyl), —CN, optionally substituted C1-C6 alkyl, and optionally substituted aryl.

One embodiment provides a pharmaceutical composition comprising a compound of Formula (I) or (II), or pharmaceutically acceptable salt or solvate thereof, and at least one pharmaceutically acceptable excipient.

One embodiment provides a method of treating a disease or disorder in a patient in need thereof comprising administering to the patient a compound of Formula (I) or (II), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### DEFINITIONS

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR, —C(O)N(R^{a})₂, —N(R^{a})C(O)OR^{a}, —OC(O)—N(R^{a})₂, —N(R^{a})C(O)R^{a}, —N(R^{a})S(O)R^{a} (where t is 1 or 2), —S(O)OR^{a} (where t is 1 or 2), —S(O)R^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R)C(O)R, —N(R^{a})S(O)ₜR^{a} (where t is 1 or 2), —S(O)ₜOR^{a} (where t is 1 or 2), —S(O)ₜR^{a} (where t is 1 or 2) and —S(O)NN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R)C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R)₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)R^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R¹ (where t is 1 or 2), —R^{b}—S(O)R¹ (where t is 1 or 2), —R^{b}—S(O)OR (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group. “Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R¹ is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —Rₜ—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R is a straight or branched alkylene or alkenylene chain, and where each of the R, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

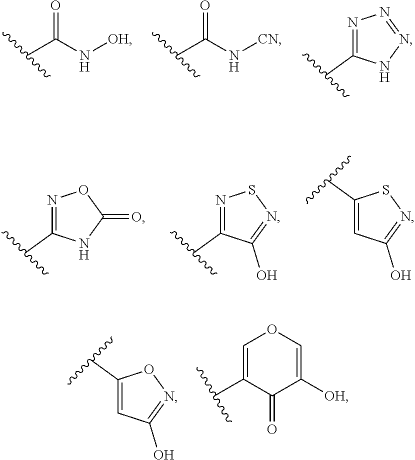

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

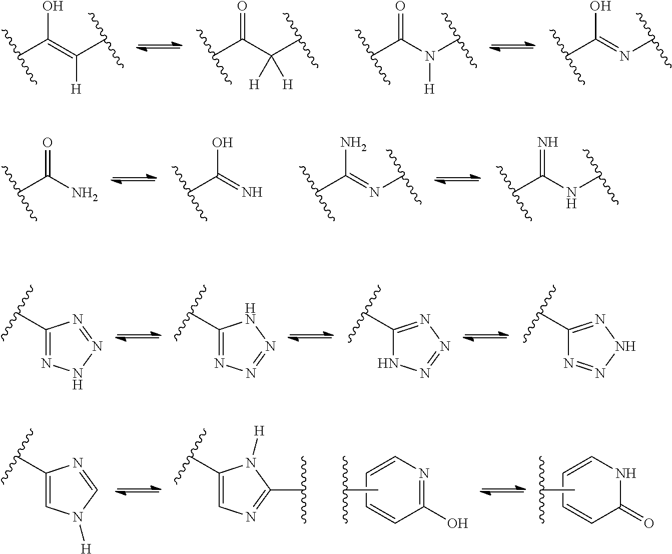

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²¹I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32. Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

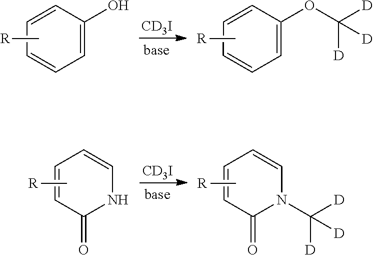

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

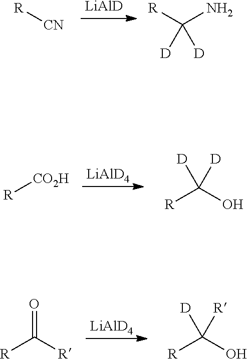

Deuterium gas and palladium catalyst are employed to reduce unsaturated carbon-carbon linkages and to perform a reductive substitution of aryl carbon-halogen bonds as illustrated, by way of example only, in the reaction schemes below.

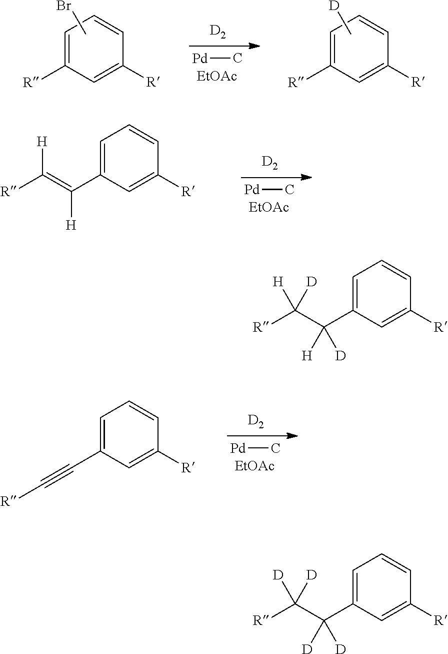

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the AKT1 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### AKT1 Protein and Function

AKT, also known as protein kinase B (PKB), is a serine/threonine protein kinase with three isoforms, AKT1, AKT2, and AKT3. While the isoforms are encoded by different genes, they are highly homologous at the protein level and share a conserved domain structure comprising an N-terminal pleckstrin homology (PH) domain, a kinase domain, and a C-terminal regulatory domain comprising a hydrophobic moiety, which includes the regulatory serine residue (Nitulescu, G. M. et al., Int J Oncol., 2018; 53(6): 2319-2331).

AKT proteins play a crucial role in major cellular functions including cell cycle progression, cell size, regulation of glucose metabolism, transcription, protein synthesis, genome stability, and neovascularization. AKT proteins can block apoptosis by inactivation of pro-apoptotic proteins, and mediate cellular growth factors, promoting cell survival. AKT is a major downstream effector of nuclear factor-kappaB (NfκB), which may link AKT signaling to the nucleus of a cell.

AKT1 is ubiquitously expressed, whereas AKT2 is primarily expressed in insulin-responsive tissues, and AKT3 is primarily expressed in brain and testes. A shared phosphorylation site of AKT in the catalytic domain corresponds to a threonine residue; specifically, Thr308 in AKT1, Thr309 in AKT2, and Thr305 in AKT3. A shared phosphorylation site in the C-terminus of the protein cis a serine residue; specifically, Ser473 in AKT1, Ser474 in AKT2, and Ser472 in AKT3.

AKT is a key downstream mediator of the phosphoinositide-3-kinase (PI3K) signaling pathway. PI3Ks are activated by different compounds. For example, PI3Kα, PI3Kβ, and PI3Kδ, are activated by extracellular ligands binding to a transmembrane glycoprotein with enzymatic activity, receptor tyrosine kinases (RTKs). In contrast, PI3Kγ is activated by G-protein-compound receptors (GPCRs) and by RAS family of GTPases.

The AKT cascade can be activated by RTKs and G-protein-compound receptors (GPCRs), along with other signals including integrins, B cell receptors, T cell receptors, and cytokine receptors.

### AKT1 Mechanism

AKT is activated by a second phosphorylation at the regulatory serine residue, Ser473. Known phosphorylating agents of AKT at Ser473 include, but are not limited to PDK-1, integrin-linked kinase (ILK), members of the PI3K-related kinase (PIKK) family, and mammalian target of rapamycin (mTOR) (Nitulescu, G. M. et al., Int J Oncol., 2018; 53(6): 2319-2331).

mTOR is a key component in the AKT signaling pathway, which is a downstream member of AKT and important regulator for cell metabolism and growth. mTOR is also an activator which can directly phosphorylate AKT's regulatory serine residue, Ser473. mTOR forms a complex with rapamycin-insensitive companion of mTOR (RICTOR) (and other proteins) to form mTOR complex 2 (mTORC2), which can directly phosphorylate AKT Ser473. AKT can affect cell survival and growth because it can influence the tuberous sclerosis complex (TSC) 1/2 along the mTORC signaling pathway and inhibit pro-apoptotic proteins or signals.

AKT is known as a survival kinase and mediates cell survival and proliferation by inhibiting pathways including, but not limited to Bcl2 and MDM2, which promotes apoptosis. Studies have shown that the AKT signaling cascade have frequent malfunctions in various cancers, and may be associated with tumor aggressiveness (Nitulescu, G. M. et al., Int J Oncol., 2018; 53(6): 2319-2331). Malfunctions of AKT typically lead to enhanced proliferation, growth, survival, and resistance to apoptosis (Alwhaibi, A. et al., Pharmacol Res., 2019, 145: 104270). Malfunction and mis-regulation of AKT may lead to cancers such as but not limited to breast cancer, gastric carcinoma, glioblastoma, gliosarcomas, head and neck squamous cell carcinoma, ovarian cancer, pancreatic cancer, and prostate cancer.

Additionally, AKT1 has been found to be involved in invasion and migration of cancerous cells (Alwhaibi, A. et al., Pharmacol Res., 2019, 145: 104270). Researchers found that silencing the AKT1 isoform can abrogate specific types of cancer cell migration. However, there have been other studies which have demonstrated that activated AKT1 resulted in less metastatic propensity for lung metastatic lesion cells and breast cancer cells. AKT1 has also been identified as a key protein involved in angiogenesis, lung cancer, and tumorigenesis.

Furthermore, overexpression of AKT has been correlated to resistance to chemotherapeutic agents such as cisplatin, methotrexate, and paclitaxel. Thus, there remains a need to find AKT inhibitors given its role in cell survival and cancer proliferation.

Recently, it has been found that the AKT1 gene mutation E17K can affect cell growth, proliferation, survival, and migration of breast cancer cells, colorectal cancer cells, and ovarian cancer cells (Chen, Y. et al., Front Cell Dev Biol., 2020; 8: 573599). These mutations in the PH structural domain increase the binding of AKT1 to Phosphatidylinositol-3,4,5-triphosphate (PIP3) lipid ligand, which accelerates transfer of AKT from the cytoplasm to the cell membrane through formation of hydrogen bonds. Transfer of AKT into the cell membrane allows it to be further phosphorylated. Once fully activated, AKT can return to the cytoplasm, or go to the nucleus or other intracellular sites, and phosphorylate other substrate proteins to regulate cell function.

The E17K mutation enhances migration of breast cancer cells, and also enhances resistance to chemotherapeutic drugs. However, the E17K mutation can also selectively destroy chemo-resistant tumor-promoting AKT1 quiescent cancer cells, suggesting that the AKT1(E17K) mutation is crucial in the oncogenic/anti-tumor mechanism.

A major pathway that activates PI3K-AKT signaling pathway is somatic cell mutations, with the E17K mutation being the highest frequency of AKT1 mutations. It is nearly exclusively present in AKT1. The AKT1(E17K) is a recurrent somatic cell mutation predominantly in breast cancer, ovarian cancer, meningioma, and Proteus syndrome.

AKT1(E17K) mutations mediate the PI3K-AKT signaling cascade by expanding PIP lipid specificity, which causes conformational changes. This also enhances subcellular localization to accelerate localization of the PH structural domain to the plasma membrane. The E17K mutation increases PIP3 binding specificity by 7-fold and phosphatidylinositol-(4,5)-bisphosphate (PIP2) by 100-fold.

The AKT1(E17K) mutation also causes rapid conformational changes in the AKT1 PH structural domain. The conformational changes to this domain result in a 4.5-fold increase in its membrane localization, which can result in excessive phosphorylation. The AKT1(E17K) mutation can also result in enhanced subcellular localization by increasing the transient expression.

Given the conformational and signaling effects of the AKT1(E17K) mutation, this target may be useful for targeted treatment of cancers.

### Prior Art AKT1 Inhibitors

Most AKT inhibitors targeting the ATP binding site are non-selective against the three isoforms, as well as having poor to no selectivity against other structurally similar kinases. Thus, there remains a need to develop new and novel AKT inhibitors. These ATP targeting inhibitors are classified as aminofurazans, azepane derivatives, isoquinoline-5-sulfonamides, phenylpyrazole derivatives, thiophene carboxamide derivatives, and thiazole carboxamide derivatives.

There are also ATP non-competitive AKT inhibitors which are allosteric modulators which has greater specificity than the ATP targeting inhibitors. Many of these allosteric modulator inhibitors are classified as purine derivatives, thiourea derivatives, alkylphospholipids, sulfonamides, 2,3-diphenylquinoxaline analogs, and indole-3-carbinol derivatives.

### Novel AKT1 Inhibitory Compounds

In one aspect, provided herein is a AKT1 inhibitory compound.

One embodiment provides a compound having the structure of Formula (I), or a pharmaceutically acceptable salt or solvate thereof:

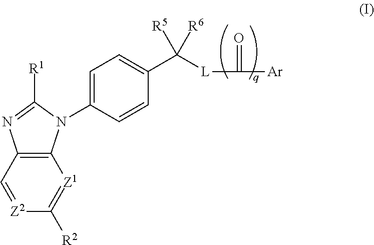

wherein:
Z¹ is N, C—H, or C—R³;
Z² is N, C—H, or C—R,
Ar is selected from

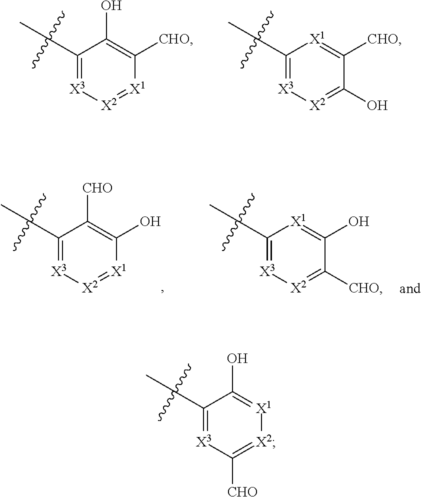

X¹ is N or C—R⁷;
X² is N or C—R⁷;
X³ is N or C—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, optionally substituted carbocyclyl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, halogen, —OH, —SH, optionally substituted C1-C6 alkoxy, —S-(optionally substituted C1-C6 alkyl), —CN, optionally substituted C1-C6 alkyl, optionally substituted amino, optionally substituted C1-C6 alkenyl, and optionally substituted aryl;
L is selected from —N(R⁸)—, —O—, or a divalent radical selected from:

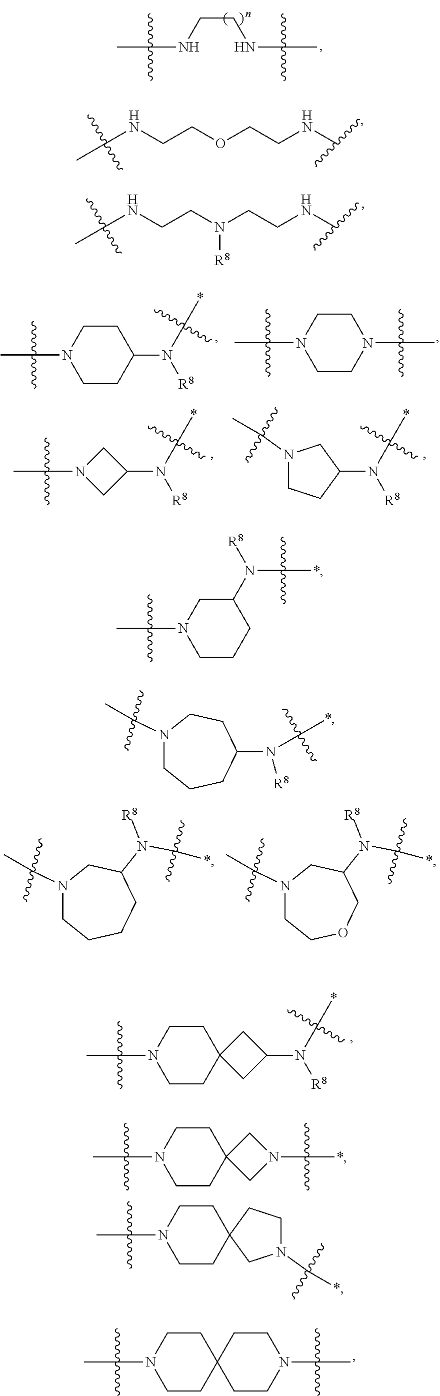

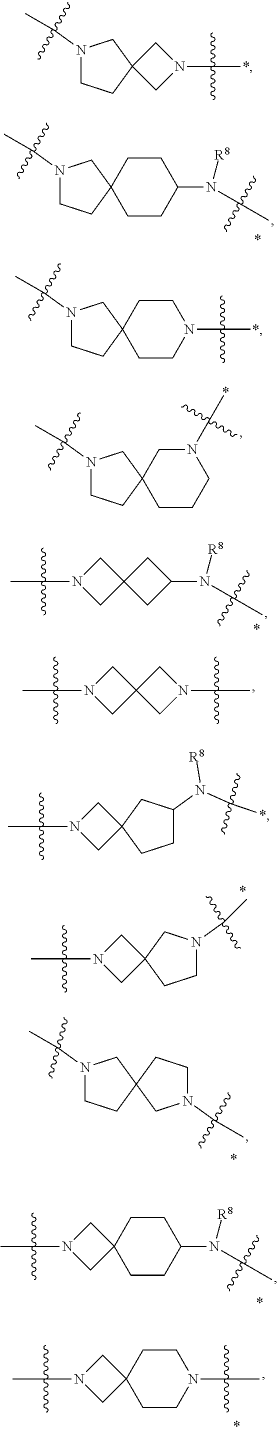

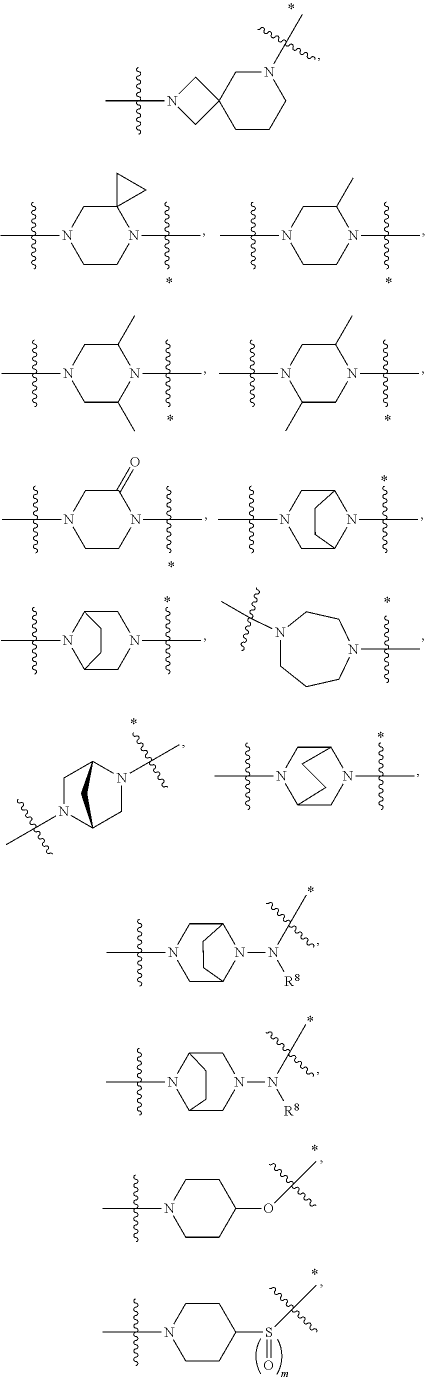

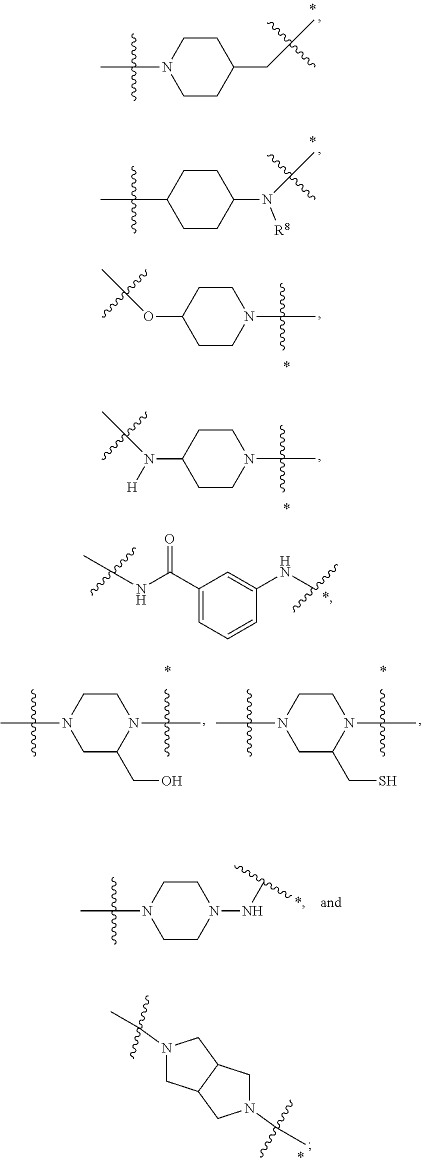

wherein the asterisk (*) indicates the bond to the —CO—Ar group;
R⁸ is hydrogen, optionally substituted C1-C6 alkyl, optionally substituted C3-C7 cycloalkyl, or optionally substituted heterocyclyl;
m is 0, 1, or 2;
n is 1, 2, or 3; and
q is 0 or 1.

One embodiment provides a compound having the structure of Formula (Ia), or a pharmaceutically acceptable salt or solvate thereof:

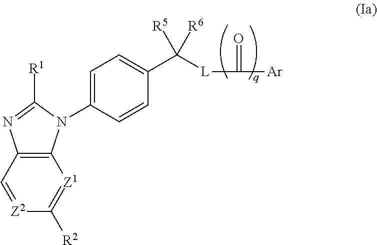

wherein:
Z¹ is N, C—H, or C—R³;
Z² is N, C—H, or C—R;
Ar is selected from

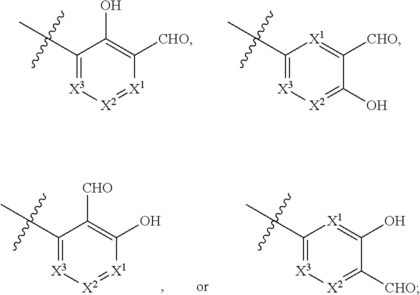

X¹ is N or C—R⁷;
X² is N or C—R⁷;
X³ is N or C—R⁷;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, halogen, —OH, —SH, optionally substituted C1-C6 alkoxy, —S-(optionally substituted C1-C6 alkyl), —CN, optionally substituted C1-C6 alkyl, and optionally substituted aryl;
L is selected from —N(R⁸)—, or a divalent radical selected from:

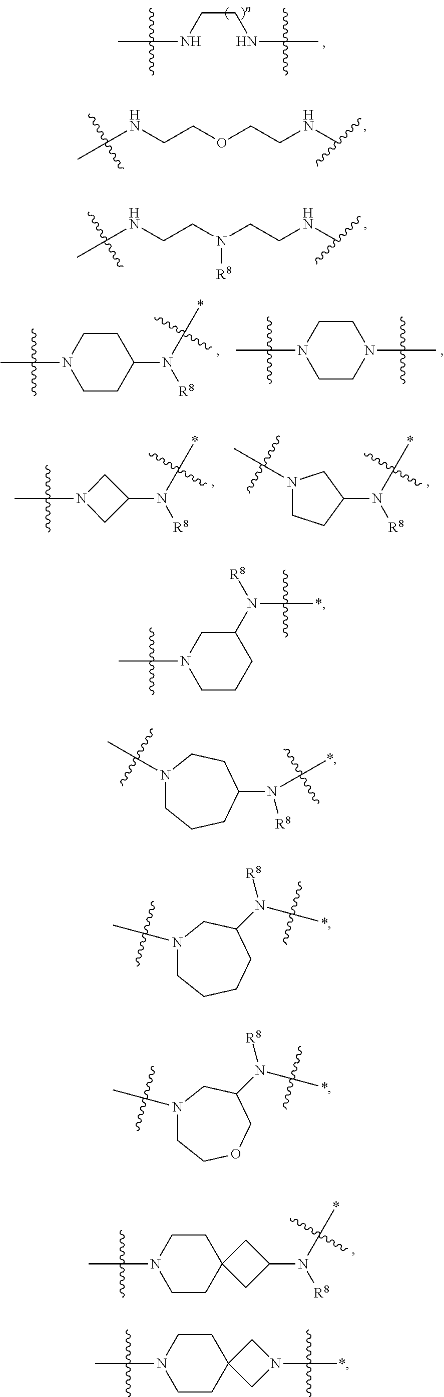

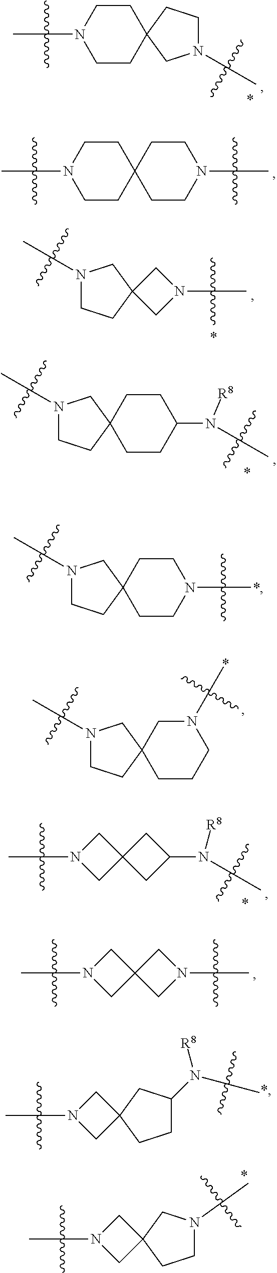

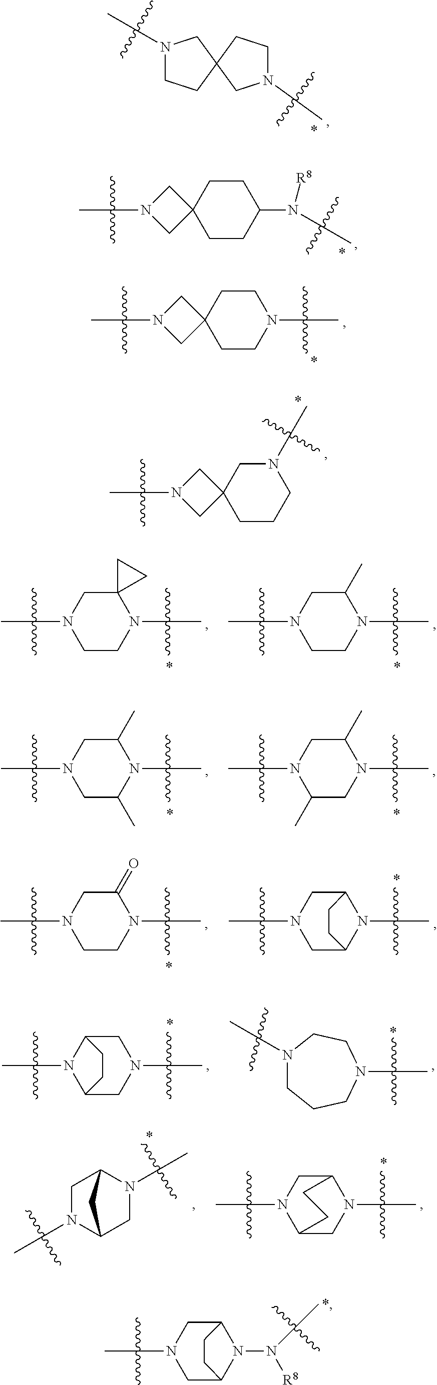

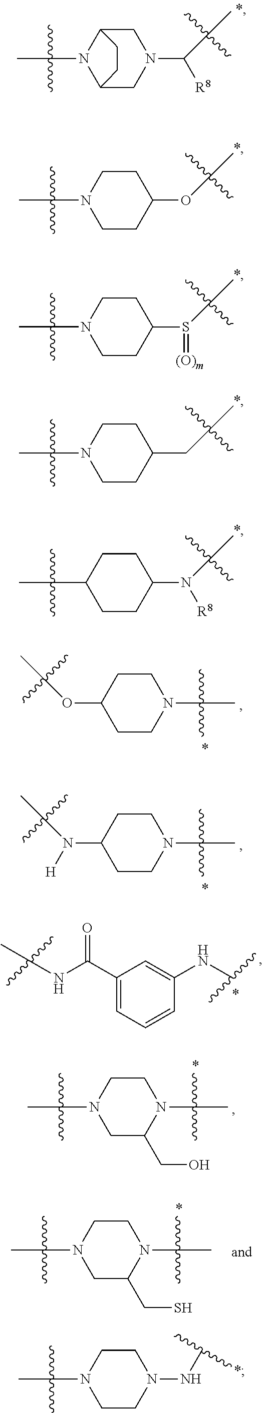

wherein the asterisk (*) indicates the bond to the —CO—Ar group;
R⁸ is hydrogen, optionally substituted C1-C6 alkyl, optionally substituted C3-C7 cycloalkyl, and optionally substituted heterocyclyl;
m is 0, 1, or 2;
n is 1, 2, or 3; and
q is 0 or 1.

Another embodiment provides a compound having the structure of Formula (I), or pharmaceutically acceptable salt or solvate thereof, wherein Z¹ is N.

Another embodiment provides a compound having the structure of Formula (I), or pharmaceutically acceptable salt or solvate thereof, wherein Z² is C—H.

Another embodiment provides a compound having the structure of Formula (I), or pharmaceutically acceptable salt or solvate thereof, wherein Z² is C—R⁴.

Another embodiment provides a compound having the structure of Formula (I), or pharmaceutically acceptable salt or solvate thereof, wherein R¹ is optionally substituted heteroaryl. Another embodiment provides a compound having the structure of Formula (I), or pharmaceutically acceptable salt or solvate thereof, wherein the optionally substituted heteroaryl is an optionally substituted pyridyl.

Another embodiment provides a compound having the structure of Formula (I), or pharmaceutically acceptable salt or solvate thereof, wherein R² is optionally substituted aryl. Another embodiment provides a compound having the structure of Formula (I), or pharmaceutically acceptable salt or solvate thereof, wherein the optionally substituted aryl is an optionally substituted phenyl.

Another embodiment provides a compound having the structure of Formula (I), or pharmaceutically acceptable salt or solvate thereof, wherein R⁵ is hydrogen.

Another embodiment provides a compound having the structure of Formula (I), or pharmaceutically acceptable salt or solvate thereof, wherein R⁶ is hydrogen.

Another embodiment provides a compound having the structure of Formula (I), or pharmaceutically acceptable salt or solvate thereof, wherein R⁵ and R⁶ together form an oxo.

Another embodiment provides a compound having the structure of Formula (I), or pharmaceutically acceptable salt or solvate thereof, wherein R⁶ is optionally substituted C1-C6 alkyl.

Another embodiment provides a compound having the structure of Formula (I), or pharmaceutically acceptable salt or solvate thereof, wherein R⁵ and R⁶ join together to form a carbocycle or heterocycle.

Another embodiment provides a compound having the structure of Formula (I), or pharmaceutically acceptable salt or solvate thereof, wherein L is —N(R⁸)—.

Another embodiment provides a compound having the structure of Formula (I), or pharmaceutically acceptable salt or solvate thereof, wherein L is selected from:

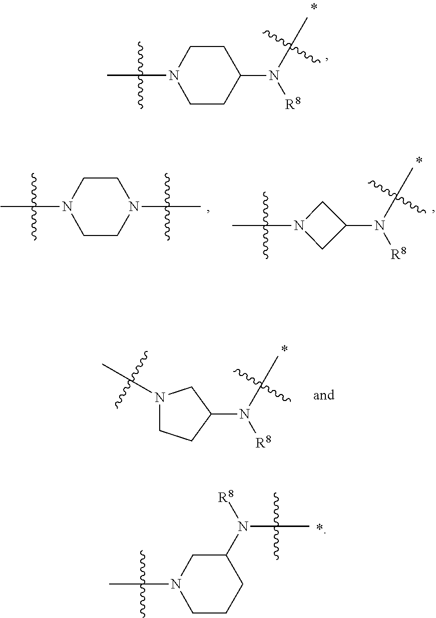

Another embodiment provides a compound having the structure of Formula (I), or pharmaceutically acceptable salt or solvate thereof, wherein L is selected from:

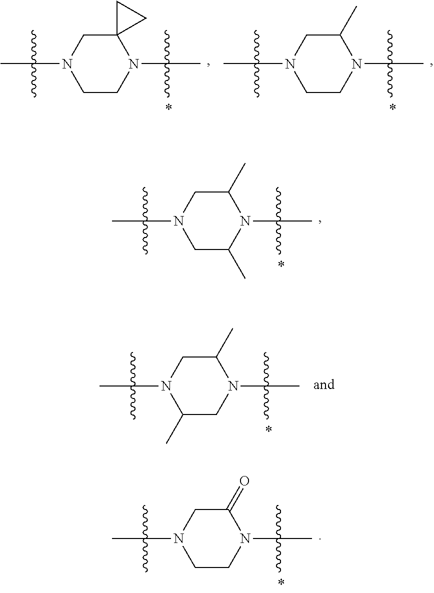

Another embodiment provides a compound having the structure of Formula (I), or pharmaceutically acceptable salt or solvate thereof, wherein L is selected from:

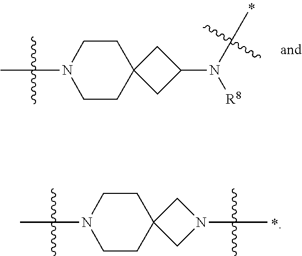

Another embodiment provides a compound having the structure of Formula (I), or pharmaceutically acceptable salt or solvate thereof, wherein L is selected from:

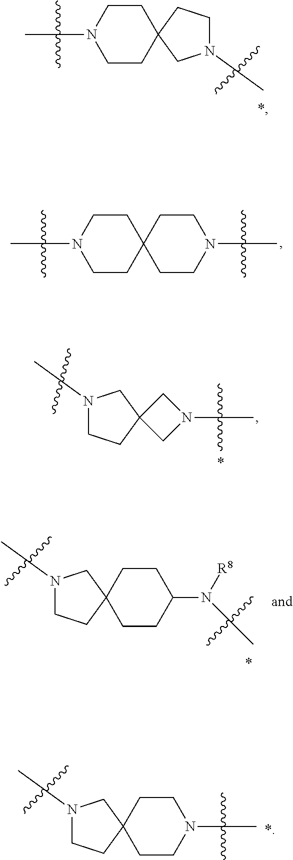

Another embodiment provides a compound having the structure of Formula (I), or pharmaceutically acceptable salt or solvate thereof, wherein L is selected from:

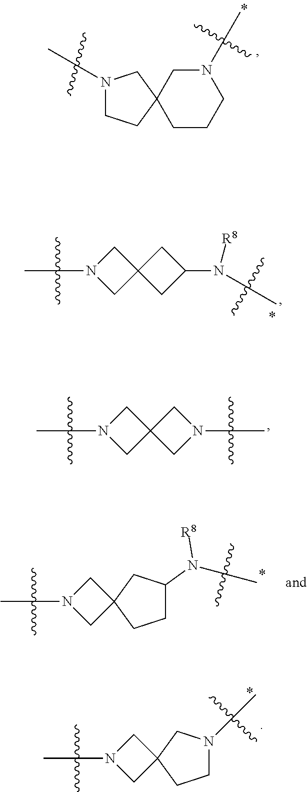

Another embodiment provides a compound having the structure of Formula (I), or pharmaceutically acceptable salt or solvate thereof, wherein L is selected from:

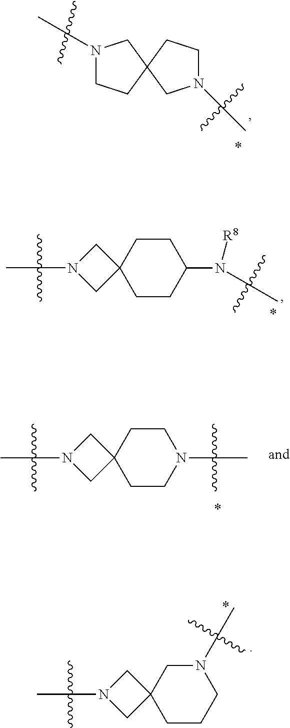

Another embodiment provides a compound having the structure of Formula (I), or pharmaceutically acceptable salt or solvate thereof, wherein R⁸ is hydrogen or optionally substituted C1-C6 alkyl.

Another embodiment provides a compound having the structure of Formula (I), or pharmaceutically acceptable salt or solvate thereof, wherein q is 0. Another embodiment provides a compound having the structure of Formula (I), or pharmaceutically acceptable salt or solvate thereof, wherein q is 1.

Another embodiment provides a compound having the structure of Formula (I), or pharmaceutically acceptable salt or solvate thereof, wherein Ar is

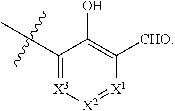

Another embodiment provides a compound having the structure of Formula (I), or pharmaceutically acceptable salt or solvate thereof, wherein Ar is

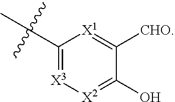

Another embodiment provides a compound having the structure of Formula (I), or pharmaceutically acceptable salt or solvate thereof, wherein Ar is

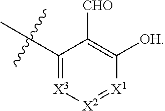

Another embodiment provides a compound having the structure of Formula (I), or pharmaceutically acceptable salt or solvate thereof, wherein Ar is

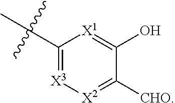

Another embodiment provides a compound having the structure of Formula (I), or pharmaceutically acceptable salt or solvate thereof, wherein X¹, X², and X³ are C—H.

One embodiment provides a compound having the structure of Formula (II), or a pharmaceutically acceptable salt or solvate thereof:

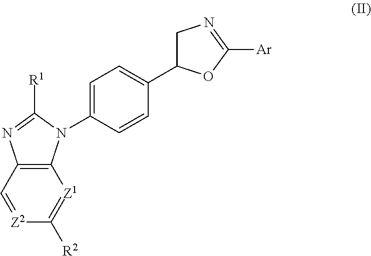

wherein:
Z¹ is N, C—H, or C—R³;
Z² is N, C—H, or C—R,
Ar is selected from

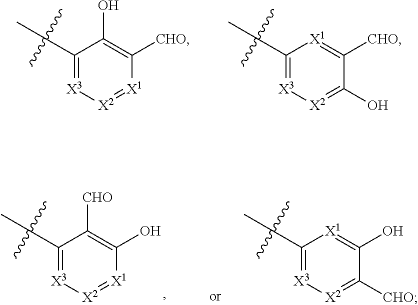

X¹ is N or C—R⁵;
X² is N or C—R⁵;
X is N or C—R⁵;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl; and
each R⁵ is independently selected from hydrogen, halogen, —OH, —SH, optionally substituted C1-C6 alkoxy, —S-(optionally substituted C1-C6 alkyl), —CN, optionally substituted C1-C6 alkyl, and optionally substituted aryl.

One embodiment provides a compound having the structure of Formula (II), or pharmaceutically acceptable salt or solvate thereof, wherein Z¹ is N.

One embodiment provides a compound having the structure of Formula (II), or pharmaceutically acceptable salt or solvate thereof, wherein Z² is C—H.

One embodiment provides a compound having the structure of Formula (II), or pharmaceutically acceptable salt or solvate thereof, wherein Z² is C—R⁴.

One embodiment provides a compound having the structure of Formula (II), or pharmaceutically acceptable salt or solvate thereof, wherein R¹ is optionally substituted heteroaryl. One embodiment provides a compound having the structure of Formula (II), or pharmaceutically acceptable salt or solvate thereof, wherein the optionally substituted heteroaryl is an optionally substituted pyridyl.

One embodiment provides a compound having the structure of Formula (II), or pharmaceutically acceptable salt or solvate thereof, wherein R² is optionally substituted aryl.

One embodiment provides a compound having the structure of Formula (II), or pharmaceutically acceptable salt or solvate thereof, wherein the optionally substituted aryl is an optionally substituted phenyl.

One embodiment provides a compound having the structure of Formula (II), or pharmaceutically acceptable salt or solvate thereof, wherein Ar is

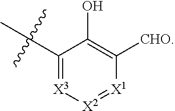

One embodiment provides a compound having the structure of Formula (II), or pharmaceutically acceptable salt or solvate thereof, wherein Ar is

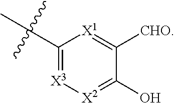

One embodiment provides a compound having the structure of Formula (II), or pharmaceutically acceptable salt or solvate thereof, wherein Ar is

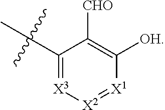

One embodiment provides a compound having the structure of Formula (II), or pharmaceutically acceptable salt or solvate thereof, wherein Ar is

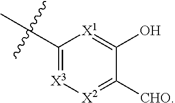

One embodiment provides a compound having the structure of Formula (II), or pharmaceutically acceptable salt or solvate thereof, wherein X¹, X², and X³ are C—H.

One embodiment provides an AKT1 inhibitory compound, or a pharmaceutically acceptable salt or solvate thereof, having a structure presented in Table 1.

TABLE 1

| Syn- | | |
|---|---|---|
| thetic | | |
| Chem- | | |
| is- | | |
| try | | |
| Ex- | | |
| ample | Compound Structure | Compound Name |
| 1 | 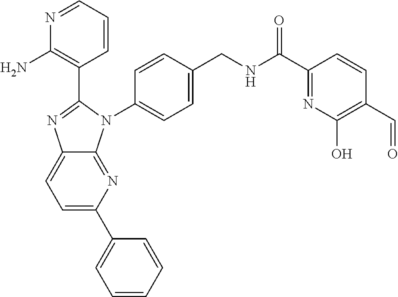 [SMILES: Nc1ncccc1-c1nc2ccc(-c3ccccc3)nc2n1-c1ccc(CNC(=O)c2ccc(C=O)c(O)n2)cc1] | N-(4-(2-(2-aminopyridin-3-yl)-5-phenyl- 3H-imidazo[4,5-b]pyridin-3-yl)benzyl)-4- formyl-3-hydroxybenzamide |
| 2 | 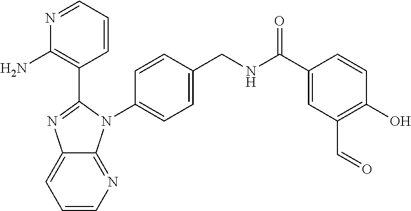 [SMILES: Nc1ncccc1-c1nc2cccnc2n1-c1ccc(CNC(=O)c2ccc(O)c(C=O)c2)cc1] | N-(4-(2-(2-aminopyridin-3-yl)-3H- imidazo[4,5-b]pyridin-3-yl)benzyl)-3- formyl-4-hydroxybenzamide |
| 3 | 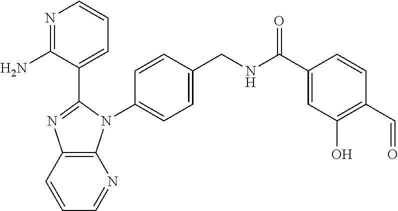 [SMILES: Nc1ncccc1-c1nc2cccnc2n1-c1ccc(CNC(=O)c2ccc(C=O)c(O)c2)cc1] | N-(4-(2-(2-aminopyridin-3-yl)-3H- imidazo[4,5-b]pyridin-3-yl)benzyl)-4- formyl-3-hydroxybenzamide |
| 4 | 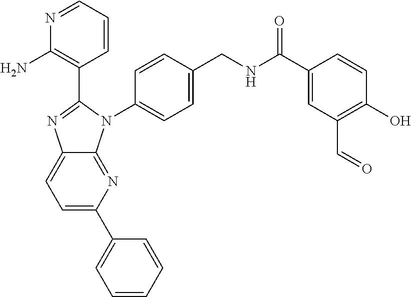 [SMILES: Nc1ncccc1-c1nc2ccc(-c3ccccc3)nc2n1-c1ccc(CNC(=O)c2ccc(O)c(C=O)c2)cc1] | N-(4-(2-(2-aminopyridin-3-yl)-5-phenyl- 3H-imidazo[4,5-b]pyridin-3-yl)benzyl)-3- formyl-4-hydroxybenzamide |
| 5 | 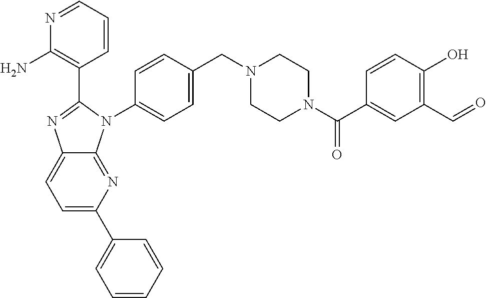 [SMILES: Nc1ncccc1-c1nc2ccc(-c3ccccc3)nc2n1-c1ccc(CN2CCN(C(=O)c3ccc(O)c(C=O)c3)CC2)cc1] | 5-(4-(4-(2-(2-aminopyridin-3-yl)-5-phenyl- 3H-imidazo[4,5-b]pyridin-3- yl)benzyl)piperazine-1-carbonyl)-2- hydroxybenzaldehyde |
| 6 | 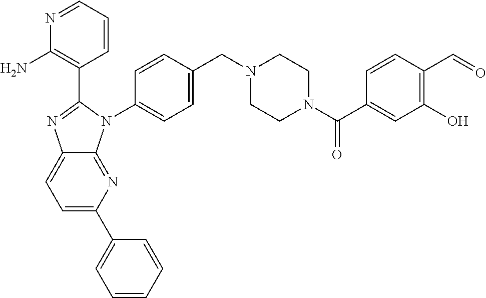 [SMILES: Nc1ncccc1-c1nc2ccc(-c3ccccc3)nc2n1-c1ccc(CN2CCN(C(=O)c3ccc(C=O)c(O)c3)CC2)cc1] | 4-(4-(4-(2-(2-aminopyridin-3-yl)-5-phenyl- 3H-imidazo[4,5-b]pyridin-3- yl)benzyl)piperazine-1-carbonyl)-2- hydroxybenzaldehyde |
| 7 | 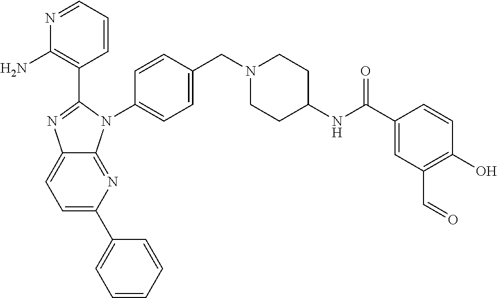 [SMILES: Nc1ncccc1-c1nc2ccc(-c3ccccc3)nc2n1-c1ccc(CN2CCC(NC(=O)c3ccc(O)c(C=O)c3)CC2)cc1] | N-(1-(4-(2-(2-aminopyridin-3-yl)-5-phenyl- 3H-imidazo[4,5-b]pyridin-3- yl)benzyl)piperidin-4-yl)-3-formy1-4- hydroxybenzamide |
| 8 | 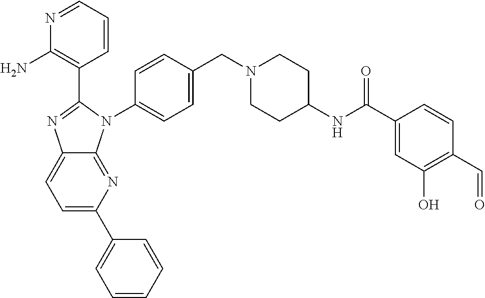 [SMILES: Nc1ncccc1-c1nc2ccc(-c3ccccc3)nc2n1-c1ccc(CN2CCC(NC(=O)c3ccc(C=O)c(O)c3)CC2)cc1] | N-(1-(4-(2-(2-aminopyridin-3-yl)-5-phenyl- 3H-imidazo[4,5-b]pyridin-3- yl)benzyl)piperidin-4-yl)-4-formyl-3- hydroxybenzamide |
| 9 | 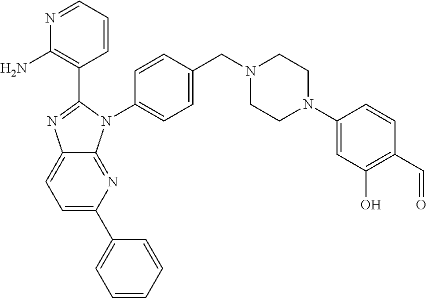 [SMILES: Nc1ncccc1-c1nc2ccc(-c3ccccc3)nc2n1-c1ccc(CN2CCN(c3ccc(C=O)c(O)c3)CC2)cc1] | 4-(4-(4-(2-(2-aminopyridin-3-yl)-5-phenyl- 3H-imidazo[4,5-b]pyridin-3- yl)benzyl)piperazin-1-yl)-2- hydroxybenzaldehyde |
| 10 | 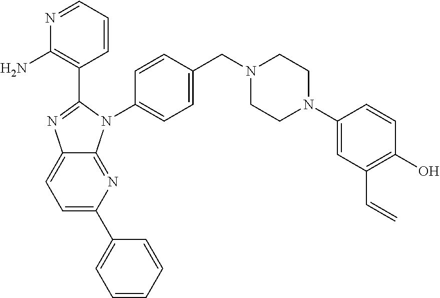 [SMILES: C=Cc1cc(N2CCN(Cc3ccc(-n4c(-c5cccnc5N)nc5ccc(-c6ccccc6)nc54)cc3)CC2)ccc1O] | 5-(4-(4-(2-(2-aminopyridin-3-yl)-5-phenyl- 3H-imidazo[4,5-b]pyridin-3- yl)benzyl)piperazin-1-yl)-2- hydroxybenzaldehyde |
| 11 | 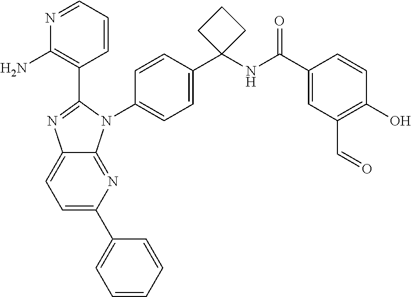 [SMILES: Nc1ncccc1-c1nc2ccc(-c3ccccc3)nc2n1-c1ccc(C2(NC(=O)c3ccc(O)c(C=O)c3)CCC2)cc1] | N-(1-(4-(2-(2-aminopyridin-3-yl)-5-phenyl- 3H-imidazo[4,5-b]pyridin-3- yl)phenyl)cyclobutyl)-3-formyl-4- hydroxybenzamide |
| 12 | 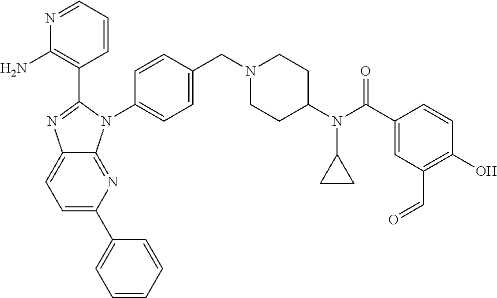 [SMILES: Nc1ncccc1-c1nc2ccc(-c3ccccc3)nc2n1-c1ccc(CN2CCC(N(C(=O)c3ccc(O)c(C=O)c3)C3CC3)CC2)cc1] | N-(1-(4-(2-(2-aminopyridin-3-yl)-5-phenyl- 3H-imidazo[4,5-b]pyridin-3- yl)benzyl)piperidin-4-yl)-N-cyclopropyl-3- formy1-4-hydroxybenzamide |
| 13 | 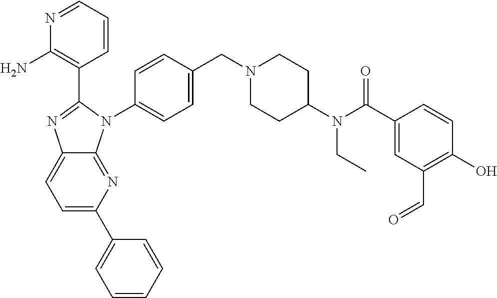 [SMILES: CCN(C(=O)c1ccc(O)c(C=O)c1)C1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-N-ethyl-3-formyl- 4-hydroxybenzamide |
| 14 | 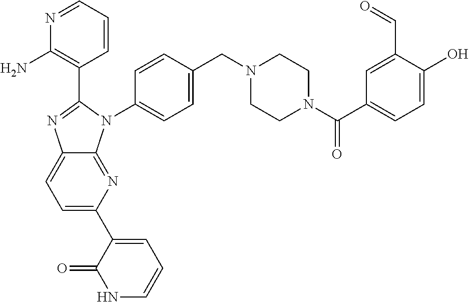 [SMILES: Nc1ncccc1-c1nc2ccc(-c3ccc[nH]c3=O)nc2n1-c1ccc(CN2CCN(C(=O)c3ccc(O)c(C=O)c3)CC2)cc1] | 5-(4-(4-(2-(2-aminopyridin-3-yl)-5-(2-oxo- 1,2-dihydropyridin-3-yl)-3H-imidazo[4,5- b]pyridin-3-yl)benzyl)piperazine-1- carbonyl)-2-hydroxybenzaldehyde |
| 15 | 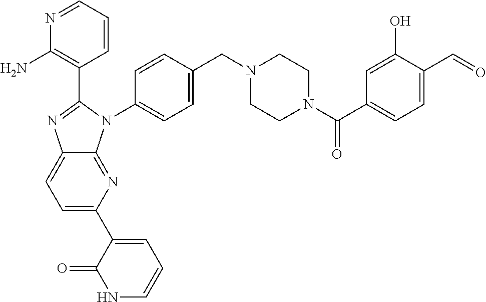 [SMILES: Nc1ncccc1-c1nc2ccc(-c3ccc[nH]c3=O)nc2n1-c1ccc(CN2CCN(C(=O)c3ccc(C=O)c(O)c3)CC2)cc1] | 4-(4-(4-(2-(2-aminopyridin-3-yl)-5-(2-oxo- 1,2-dihydropyridin-3-yl)-3H-imidazo[4,5- b]pyridin-3-yl)benzyl)piperazine-1- carbonyl)-2-hydroxybenzaldehyde |
| 16 | 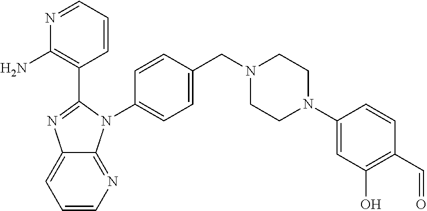 [SMILES: Nc1ncccc1-c1nc2cccnc2n1-c1ccc(CN2CCN(c3ccc(C=O)c(O)c3)CC2)cc1] | 4-(4-(4-(2-(2-aminopyridin-3-yl)-3H- imidazo[4,5-b]pyridin-3- yl)benzyl)piperazin- 1-yl)-2-hydroxybenzaldehyde |
| 17 | 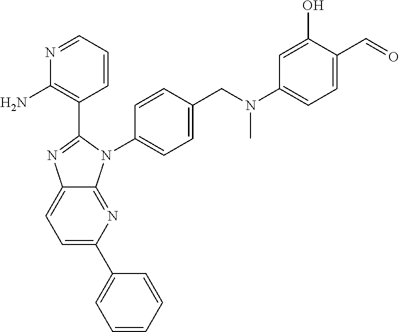 [SMILES: CN(Cc1ccc(-n2c(-c3cccnc3N)nc3ccc(-c4ccccc4)nc32)cc1)c1ccc(C=O)c(O)c1] | 4-((4-(2-(2-aminopyridin-3- yl)-5-phenyl-3H- imidazo[4,5-b]pyridin-3- yl)benzyl)(methyl)amino)-2- hydroxybenzaldehyde |
| 18 | 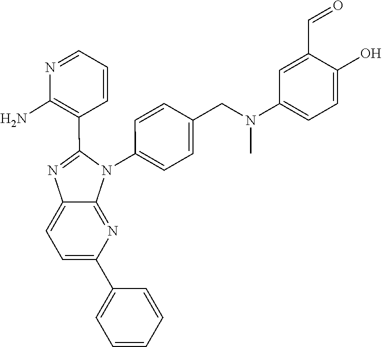 [SMILES: CN(Cc1ccc(-n2c(-c3cccnc3N)nc3ccc(-c4ccccc4)nc32)cc1)c1ccc(O)c(C=O)c1] | 5-((4-(2-(2-aminopyridin-3-yl)- 5-phenyl-3H- imidazo[4,5-b]pyridin-3- yl)benzyl)(methyl)amino)-2- hydroxybenzaldehyde |
| 19 | 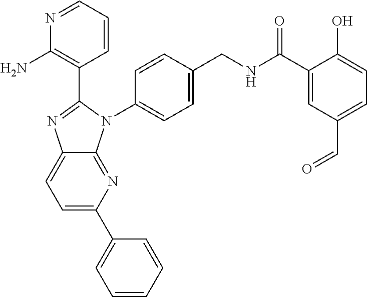 [SMILES: Nc1ncccc1-c1nc2ccc(-c3ccccc3)nc2n1-c1ccc(CNC(=O)c2cc(C=O)ccc2O)cc1] | N-(4-(2-(2-aminopyridin-3- yl)-5-phenyl-3H- imidazo[4,5-b]pyridin-3-yl)benzyl)-5- formyl-2-hydroxybenzamide |
| 20 | 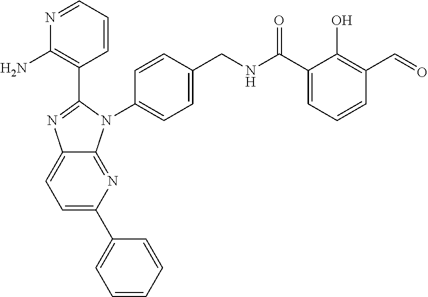 [SMILES: Nc1ncccc1-c1nc2ccc(-c3ccccc3)nc2n1-c1ccc(CNC(=O)c2cccc(C=O)c2O)cc1] | N-(4-(2-(2-aminopyridin-3- yl)-5-phenyl-3H- imidazo[4,5-b]pyridin-3-yl)benzyl)-3- formyl-2-hydroxybenzamide |
| 21 | 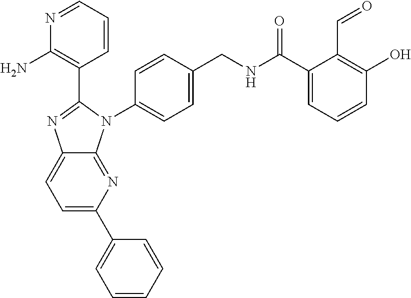 [SMILES: Nc1ncccc1-c1nc2ccc(-c3ccccc3)nc2n1-c1ccc(CNC(=O)c2cccc(O)c2C=O)cc1] | N-(4-(2-(2-aminopyridin- 3-yl)-5-phenyl-3H- imidazo[4,5-b]pyridin-3-yl)benzyl)-2- formyl-3-hydroxybenzamide |
| 22 | 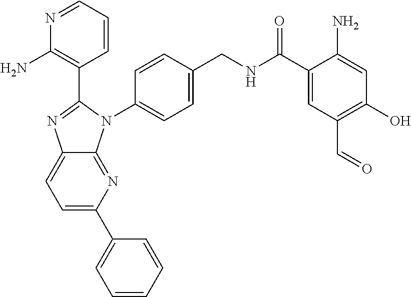 [SMILES: Nc1cc(O)c(C=O)cc1C(=O)NCc1ccc(-n2c(-c3cccnc3N)nc3ccc(-c4ccccc4)nc32)cc1] | 2-amino-N-(4-(2-(2-amino- pyridin-3-yl)-5- phenyl-3H-imidazo[4,5-b]pyridin-3- yl)benzyl)-5-formyl-4-hydroxybenzamide |
| 23 | 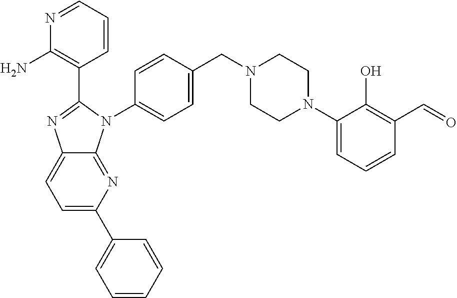 [SMILES: Nc1ncccc1-c1nc2ccc(-c3ccccc3)nc2n1-c1ccc(CN2CCN(c3cccc(C=O)c3O)CC2)cc1] | 3-(4-(4-(2-(2-aminopyridin-3-yl)-5-phenyl- 3H-imidazo[4,5-b]pyridin-3- yl)benzyl)piperazin-1-yl)-2- hydroxybenzaldehyde |
| 24 | 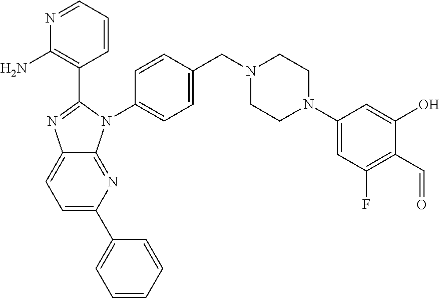 [SMILES: Nc1ncccc1-c1nc2ccc(-c3ccccc3)nc2n1-c1ccc(CN2CCN(c3cc(O)c(C=O)c(F)c3)CC2)cc1] | 4-(4-(4-(2-(2-aminopyridin-3-yl)-5-phenyl- 3H-imidazo[4,5-b]pyridin-3- yl)benzyl)piperazin-1-yl)-2-fluoro-6- hydroxybenzaldehyde |
| 25 | 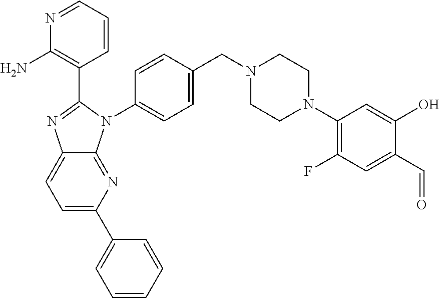 [SMILES: Nc1ncccc1-c1nc2ccc(-c3ccccc3)nc2n1-c1ccc(CN2CCN(c3cc(O)c(C=O)cc3F)CC2)cc1] | 4-(4-(4-(2-(2-aminopyridin-3-yl)-5-phenyl- 3H-imidazo[4,5-b]pyridin-3- yl)benzyl)piperazin-1-yl)-5-fluoro-2- hydroxybenzaldehyde |
| 26 | 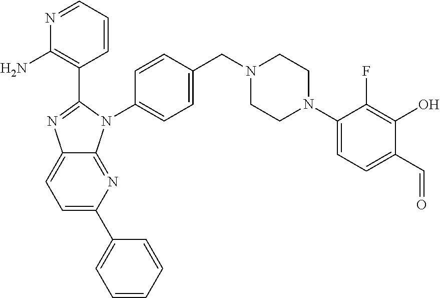 [SMILES: Nc1ncccc1-c1nc2ccc(-c3ccccc3)nc2n1-c1ccc(CN2CCN(c3ccc(C=O)c(O)c3F)CC2)cc1] | 4-(4-(4-(2-(2-aminopyridin-3-yl)-5-phenyl- 3H-imidazo[4,5-b]pyridin-3- yl)benzyl)piperazin-1-yl)-3-fluoro-2- hydroxybenzaldehyde |
| 27 | 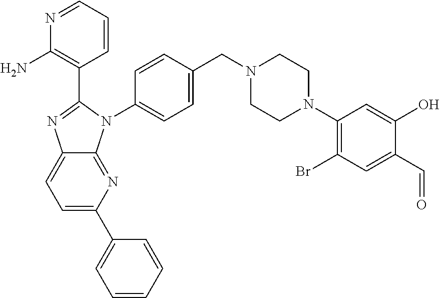 [SMILES: Nc1ncccc1-c1nc2ccc(-c3ccccc3)nc2n1-c1ccc(CN2CCN(c3cc(O)c(C=O)cc3Br)CC2)cc1] | 4-(4-(4-(2-(2-aminopyridin-3-yl)-5-phenyl- 3H-imidazo[4,5-b]pyridin-3- yl)benzyl)piperazin-1-yl)-5-bromo-2- hydroxybenzaldehyde |
| 28 | 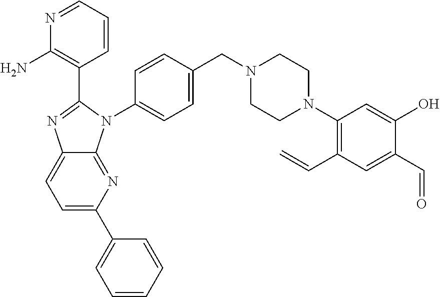 [SMILES: C=Cc1cc(C=O)c(O)cc1N1CCN(Cc2ccc(-n3c(-c4cccnc4N)nc4ccc(-c5ccccc5)nc43)cc2)CC1] | 4-(4-(4-(2-(2-aminopyridin-3-yl)-5-phenyl- 3H-imidazo[4,5-b]pyridin-3- yl)benzyl)piperazin-1-yl)-2-hydroxy-5- vinylbenzaldehyde |
| 29 | 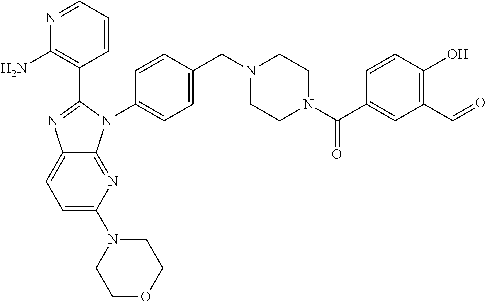 [SMILES: Nc1ncccc1-c1nc2ccc(N3CCOCC3)nc2n1-c1ccc(CN2CCN(C(=O)c3ccc(O)c(C=O)c3)CC2)cc1] | 5-(4-(4-(2-(2-aminopyridin-3-yl)-5-(3,6- dihydro-2H-pyran-4-yl)-3H-imidazo[4,5- b]pyridin-3-yl)benzyl)piperazine-1- carbonyl)-2-hydroxybenzaldehyde |
| 30 | 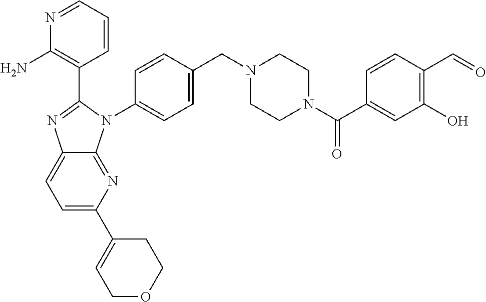 [SMILES: Nc1ncccc1-c1nc2ccc(C3=CCOCC3)nc2n1-c1ccc(CN2CCN(C(=O)c3ccc(C=O)c(O)c3)CC2)cc1] | 4-(4-(4-(2-(2-aminopyridin-3-yl)-5-(3,6- dihydro-2H-pyran-4-yl)-3H-imidazo[4,5- b]pyridin-3-yl)benzyl)piperazine-1- carbonyl)-2-hydroxybenzaldehyde |
| 31 | 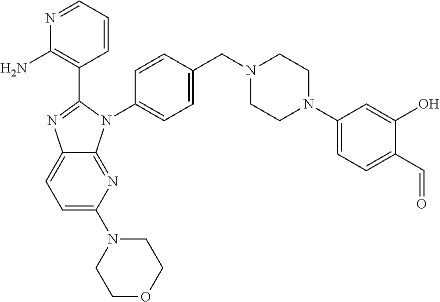 [SMILES: Nc1ncccc1-c1nc2ccc(N3CCOCC3)nc2n1-c1ccc(CN2CCN(c3ccc(C=O)c(O)c3)CC2)cc1] | 4-(4-(4-(2-(2-aminopyridin-3-y1)-5- morpholino-3H-imidazo[4,5-b]pyridin-3- yl)benzyl)piperazin-1-yl)-2- hydroxybenzaldehyde |
| 32 | 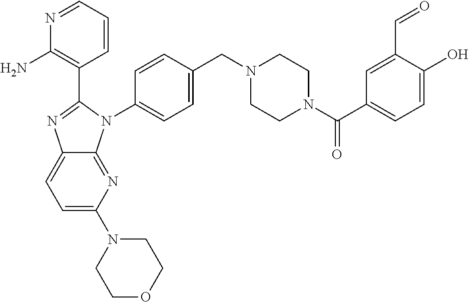 [SMILES: Nc1ncccc1-c1nc2ccc(N3CCOCC3)nc2n1-c1ccc(CN2CCN(C(=O)c3ccc(O)c(C=O)c3)CC2)cc1] | 5-(4-(4-(2-(2-aminopyridin-3-yl)-5- morpholino-3H-imidazo[4,5-b]pyridin-3- yl)benzyl)piperazine-1-carbonyl)-2- hydroxybenzaldehyde |
| 33 | 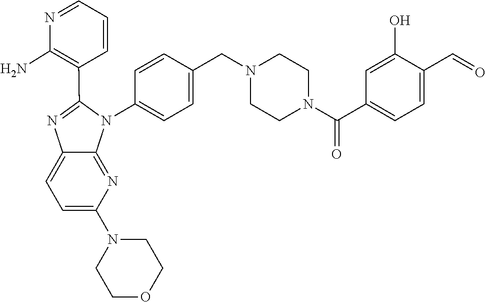 [SMILES: Nc1ncccc1-c1nc2ccc(N3CCOCC3)nc2n1-c1ccc(CN2CCN(C(=O)c3ccc(C=O)c(O)c3)CC2)cc1] | 4-(4-(4-(2-(2-aminopyridin-3-yl)-5- morpholino-3H-imidazo[4,5-b]pyridin-3- yl)benzyl)piperazine-1-carbonyl)-2- hydroxybenzaldehyde |
| 34 | 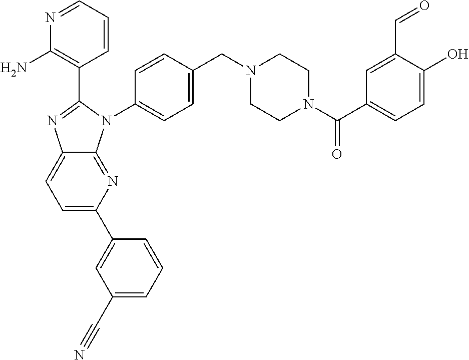 [SMILES: N#Cc1cccc(-c2ccc3nc(-c4cccnc4N)n(-c4ccc(CN5CCN(C(=O)c6ccc(O)c(C=O)c6)CC5)cc4)c3n2)c1] | 3-(2-(2-aminopyridin-3-yl)-3-(4-((4-(3- formyl-4-hydroxybenzoyl)piperazin-1- yl)methyl)phenyl)-3H-imidazo[4,5- b]pyridin-5-yl)benzonitrile |
| 35 | 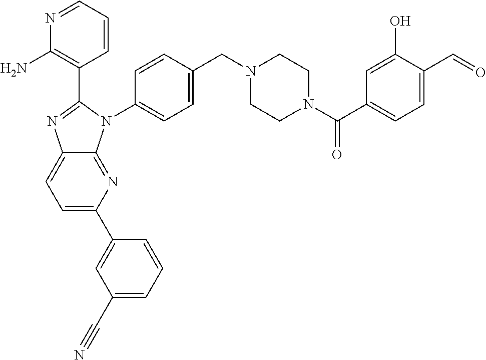 [SMILES: N#Cc1cccc(-c2ccc3nc(-c4cccnc4N)n(-c4ccc(CN5CCN(C(=O)c6ccc(C=O)c(O)c6)CC5)cc4)c3n2)c1] | 3-(2-(2-aminopyridin-3-y1)-3-(4-((4-(4- formyl-3-hydroxybenzoyl)piperazin-1- yl)methyl)phenyl)-3H-imidazo[4,5- b]pyridin-5-yl)benzonitrile |
| 36 | 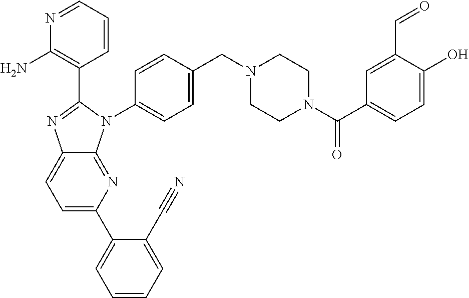 [SMILES: N#Cc1ccccc1-c1ccc2nc(-c3cccnc3N)n(-c3ccc(CN4CCN(C(=O)c5ccc(O)c(C=O)c5)CC4)cc3)c2n1] | 2-(2-(2-aminopyridin-3-yl)-3-(4-((4-(3- formyl-4-hydroxybenzoyl)piperazin-1- yl)methyl)phenyl)-3H-imidazo[4,5- b]pyridin-5-yl)benzonitrile |
| 37 | 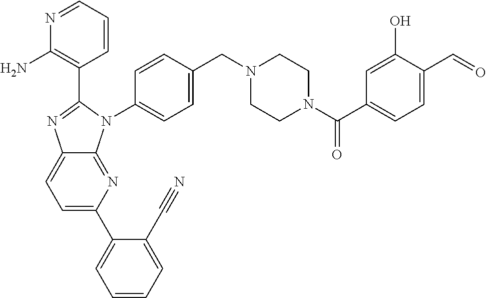 [SMILES: N#Cc1ccccc1-c1ccc2nc(-c3cccnc3N)n(-c3ccc(CN4CCN(C(=O)c5ccc(C=O)c(O)c5)CC4)cc3)c2n1] | 2-(2-(2-aminopyridin-3-yl)-3-(4-((4-(4- formyl-3-hydroxybenzoyl)piperazin-1- yl)methyl)phenyl)-3H-imidazo[4,5- b]pyridin-5-yl)benzonitrile |
| 38 | 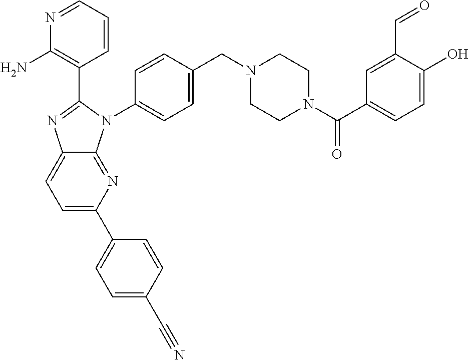 [SMILES: N#Cc1ccc(-c2ccc3nc(-c4cccnc4N)n(-c4ccc(CN5CCN(C(=O)c6ccc(O)c(C=O)c6)CC5)cc4)c3n2)cc1] | 4-(2-(2-aminopyridin-3-yl)-3-(4-((4-(3- formyl-4-hydroxybenzoyl)piperazin-1- yl)methyl)phenyl)-3H-imidazo[4,5- b]pyridin-5-yl)benzonitrile |
| 39 | 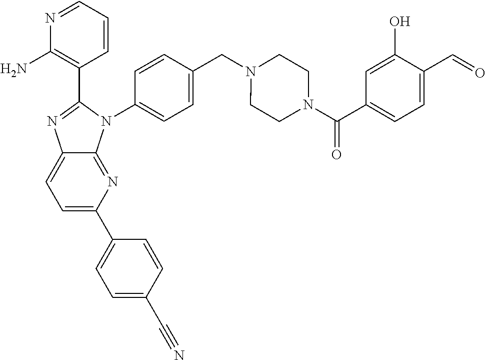 [SMILES: N#Cc1ccc(-c2ccc3nc(-c4cccnc4N)n(-c4ccc(CN5CCN(C(=O)c6ccc(C=O)c(O)c6)CC5)cc4)c3n2)cc1] | 4-(2-(2-aminopyridin-3-yl)-3-(4-((4-(4- formyl-3-hydroxybenzoyl)piperazin-1- yl)methyl)phenyl)-3H-imidazo[4,5- b]pyridin-5-yl)benzonitrile |
| 40 | 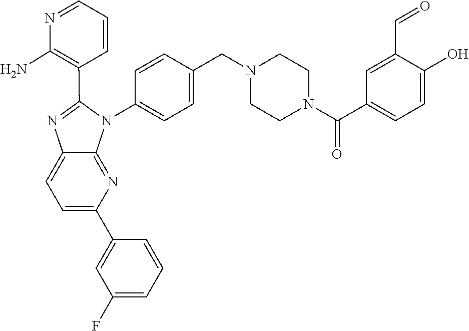 [SMILES: Nc1ncccc1-c1nc2ccc(-c3cccc(F)c3)nc2n1-c1ccc(CN2CCN(C(=O)c3ccc(O)c(C=O)c3)CC2)cc1] | 5-(4-(4-(2-(2-aminopyridin-3-yl)-5-(3- fluorophenyl)-3H-imidazo[4,5-b]pyridin-3- yl)benzyl)piperazine-1-carbonyl)-2- hydroxybenzaldehyde |
| 41 | 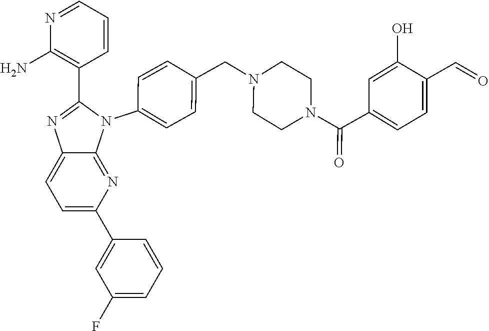 [SMILES: Nc1ncccc1-c1nc2ccc(-c3cccc(F)c3)nc2n1-c1ccc(CN2CCN(C(=O)c3ccc(C=O)c(O)c3)CC2)cc1] | 4-(4-(4-(2-(2-aminopyridin-3-yl)-5-(3- fluorophenyl)-3H-imidazo[4,5-b]pyridin-3- yl)benzyl)piperazine-1-carbonyl)-2- hydroxybenzaldehyde |
| 42 | 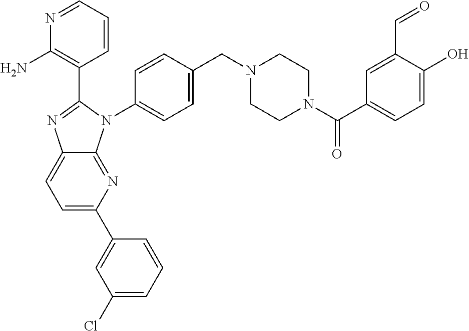 [SMILES: Nc1ncccc1-c1nc2ccc(-c3cccc(Cl)c3)nc2n1-c1ccc(CN2CCN(C(=O)c3ccc(O)c(C=O)c3)CC2)cc1] | 5-(4-(4-(2-(2-aminopyridin-3-yl)-5-(3- chlorophenyl)-3H-imidazo[4,5-b]pyridin-3- yl)benzyl)piperazine-1-carbonyl)-2- hydroxybenzaldehyde |
| 43 | 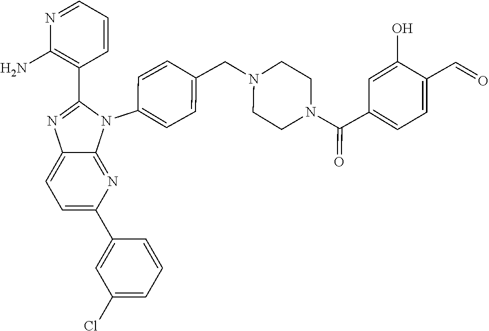 [SMILES: Nc1ncccc1-c1nc2ccc(-c3cccc(Cl)c3)nc2n1-c1ccc(CN2CCN(C(=O)c3ccc(C=O)c(O)c3)CC2)cc1] | 4-(4-(4-(2-(2-aminopyridin-3-yl)-5-(3- chlorophenyl)-3H-imidazo[4,5-b]pyridin-3- yl)benzyl)piperazine-1-carbonyl)-2- hydroxybenzaldehyde |
| 44 | 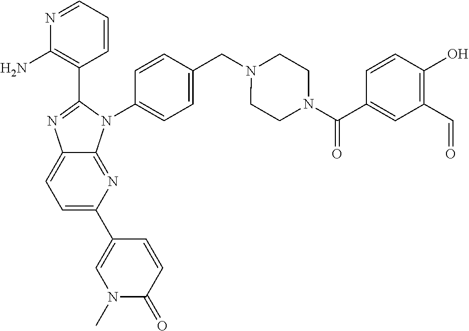 [SMILES: Cn1cc(-c2ccc3nc(-c4cccnc4N)n(-c4ccc(CN5CCN(C(=O)c6ccc(O)c(C=O)c6)CC5)cc4)c3n2)ccc1=O] | 5-(4-(4-(2-(2-aminopyridin-3-yl)-5-(1- methyl-6-oxo-1,6-dihydropyridin-3-yl)-3H- imidazo[4,5-b]pyridin-3- yl)benzyl)piperazine-1-carbonyl)-2- hydroxybenzaldehyde |
| 45 | 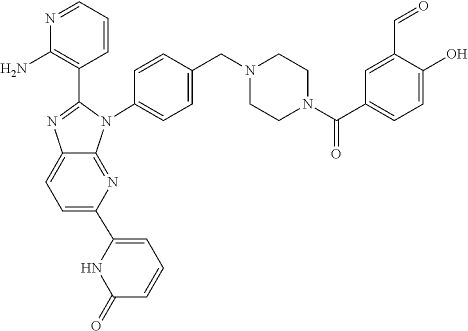 [SMILES: Nc1ncccc1-c1nc2ccc(-c3cccc(=O)[nH]3)nc2n1-c1ccc(CN2CCN(C(=O)c3ccc(O)c(C=O)c3)CC2)cc1] | 5-(4-(4-(2-(2-aminopyridin-3-yl)-5-(6-oxo- 1,6-dihydropyridin-2-yl)-3H-imidazo[4,5- b]pyridin-3-yl)benzyl)piperazine-1- carbonyl)-2-hydroxybenzaldehyde |
| 46 | 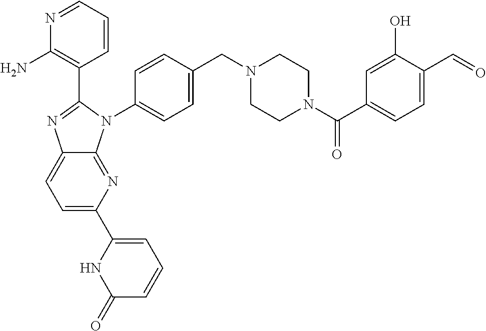 [SMILES: Nc1ncccc1-c1nc2ccc(-c3cccc(=O)[nH]3)nc2n1-c1ccc(CN2CCN(C(=O)c3ccc(C=O)c(O)c3)CC2)cc1] | 4-(4-(4-(2-(2-aminopyridin-3-yl)-5-(6-oxo- 1,6-dihydropyridin-2-yl)-3H-imidazo[4,5- b]pyridin-3-yl)benzyl)piperazine-1- carbonyl)-2-hydroxybenzaldehyde |
| 47 | 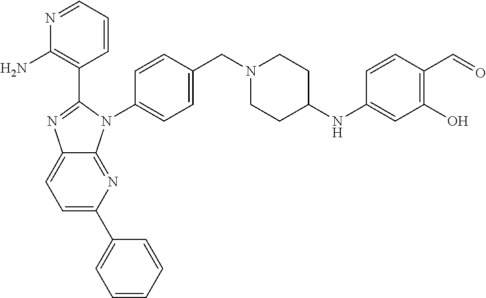 [SMILES: Nc1ncccc1-c1nc2ccc(-c3ccccc3)nc2n1-c1ccc(CN2CCC(Nc3ccc(C=O)c(O)c3)CC2)cc1] | 4-((1-(4-(2-(2-aminopyridin-3-yl)-5-phenyl- 3H-imidazo[4,5-b]pyridin-3- yl)benzyl)piperidin-4-yl)amino)-2- hydroxybenzaldehyde |
| 48 | 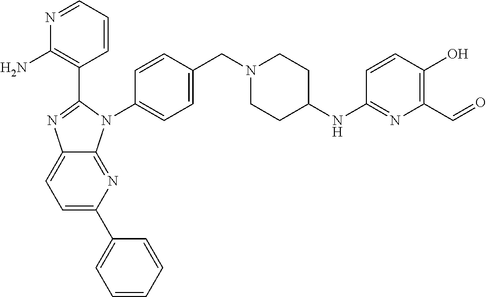 [SMILES: Nc1ncccc1-c1nc2ccc(-c3ccccc3)nc2n1-c1ccc(CN2CCC(Nc3ccc(O)c(C=O)n3)CC2)cc1] | 5-((1-(4-(2-(2-aminopyridin-3-yl)-5-phenyl- 3H-imidazo[4,5-b]pyridin-3- yl)benzyl)piperidin-4-yl)amino)-2- hydroxybenzaldehyde |
| 49 | 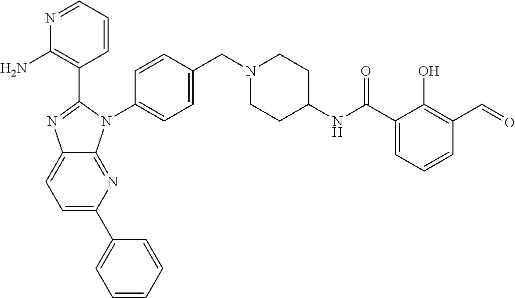 [SMILES: Nc1ncccc1-c1nc2ccc(-c3ccccc3)nc2n1-c1ccc(CN2CCC(NC(=O)c3cccc(C=O)c3O)CC2)cc1] | N-(1-(4-(2-(2-aminopyridin-3-yl)-5-phenyl- 3H-imidazo[4,5-b]pyridin-3- yl)benzyl)piperidin-4-yl)-3-formyl-2- hydroxybenzamide |
| 50 | 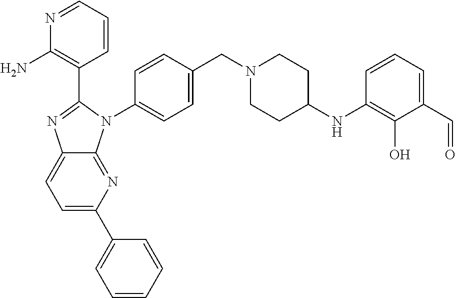 [SMILES: Nc1ncccc1-c1nc2ccc(-c3ccccc3)nc2n1-c1ccc(CN2CCC(Nc3cccc(C=O)c3O)CC2)cc1] | 3-((1-(4-(2-(2-aminopyridin-3-yl)-5-phenyl- 3H-imidazo[4,5-b]pyridin-3- yl)benzyl)piperidin-4-yl)amino)-2- hydroxybenzaldehyde |
| 51 | 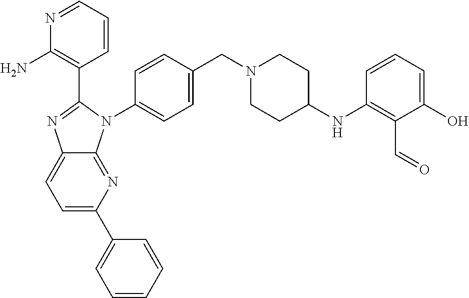 [SMILES: Nc1ncccc1-c1nc2ccc(-c3ccccc3)nc2n1-c1ccc(CN2CCC(Nc3cccc(O)c3C=O)CC2)cc1] | 2-((1-(4-(2-(2-aminopyridin-3-yl)-5-phenyl- 3H-imidazo[4,5-b]pyridin-3- yl)benzyl)piperidin-4-yl)amino)-6- hydroxybenzaldehyde |
| 52 | 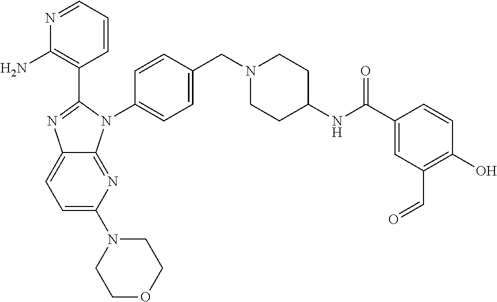 [SMILES: Nc1ncccc1-c1nc2ccc(N3CCOCC3)nc2n1-c1ccc(CN2CCC(NC(=O)c3ccc(O)c(C=O)c3)CC2)cc1] | N-(1-(4-(2-(2-aminopyridin-3-yl)-5- morpholino-3H-imidazo[4,5-b]pyridin-3- yl)benzyl)piperidin-4-yl)-3-formyl-4- hydroxybenzamide |
| 53 | 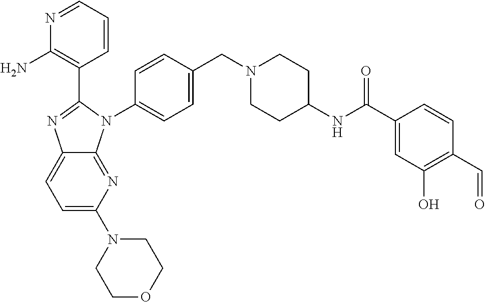 [SMILES: Nc1ncccc1-c1nc2ccc(N3CCOCC3)nc2n1-c1ccc(CN2CCC(NC(=O)c3ccc(C=O)c(O)c3)CC2)cc1] | N-(1-(4-(2-(2-aminopyridin-3-yl)-5- morpholino-3H-imidazo[4,5-b]pyridin-3- yl)benzyl)piperidin-4-yl)-4-formyl-3- hydroxybenzamide |
| 54 | 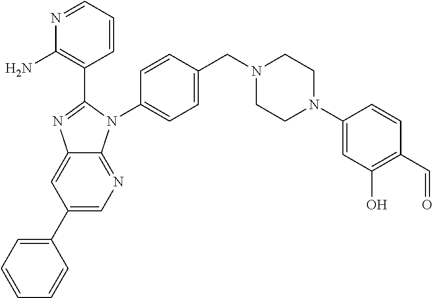 [SMILES: Nc1ncccc1-c1nc2cc(-c3ccccc3)cnc2n1-c1ccc(CN2CCN(c3ccc(C=O)c(O)c3)CC2)cc1] | 4-(4-(4-(2-(2-aminopyridin-3-yl)-6-phenyl- 3H-imidazo[4,5-b]pyridin-3- yl)benzyl)piperazin-1-yl)-2- hydroxybenzaldehyde |
| 55 | 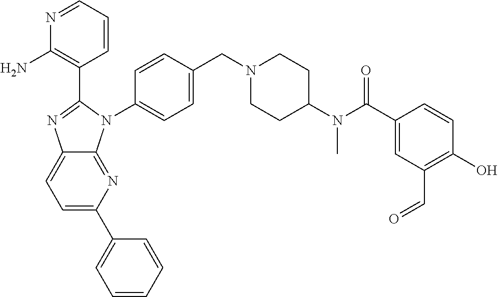 [SMILES: CN(C(=O)c1ccc(O)c(C=O)c1)C1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-3-formyl-4- hydroxy-N-methylbenzamide |
| 56 | 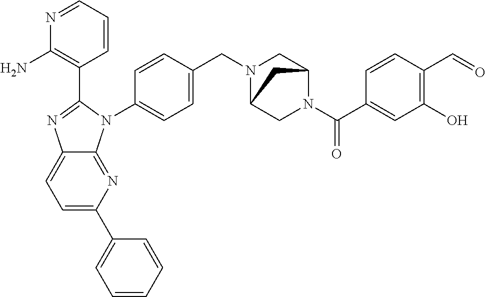 [SMILES: Nc1ncccc1-c1nc2ccc(-c3ccccc3)nc2n1-c1ccc(CN2C[C@H]3C[C@@H]2CN3C(=O)c2ccc(C=O)c(O)c2)cc1] | 4-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptane- 2-carbonyl)-2-hydroxybenzaldehyde |
| 57 | 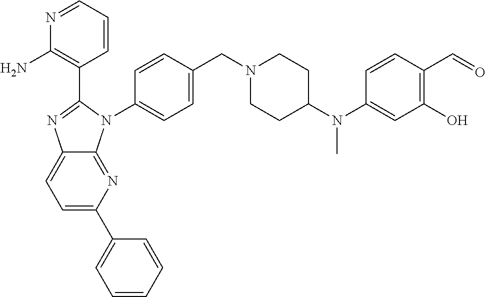 [SMILES: CN(c1ccc(C=O)c(O)c1)C1CCN(Cc2ccc(-n3c(-c4cccnc4N)nc4ccc(-c5ccccc5)nc43)cc2)CC1] | 4-((1-(4-(2-(2-aminopyridin-3-yl)-5- phenyl-3H-imidazo[4,5-b]pyridin-3- yl)benzyl)piperidin-4-yl)(methyl)amino)- 2-hydroxybenzaldehyde |
| 58 | 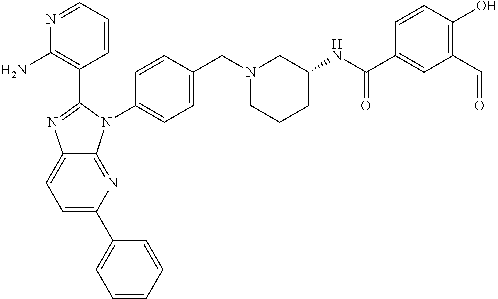 [SMILES: Nc1ncccc1-c1nc2ccc(-c3ccccc3)nc2n1-c1ccc(CN2CCC[C@@H](NC(=O)c3ccc(O)c(C=O)c3)C2)cc1] | (R)-N-(1-(4-(2-(2-aminopyridin-3-yl)-5- phenyl-3H-imidazo[4,5-b]pyridin-3- yl)benzyl)piperidin-3-yl)-3-formyl-4- hydroxybenzamide |
| 59 | 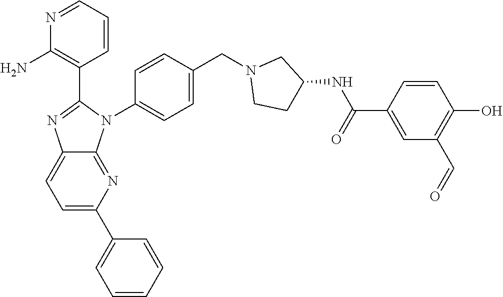 [SMILES: Nc1ncccc1-c1nc2ccc(-c3ccccc3)nc2n1-c1ccc(CN2CC[C@@H](NC(=O)c3ccc(O)c(C=O)c3)C2)cc1] | (R)-N-(1-(4-(2-(2-aminopyridin-3-yl)-5- phenyl-3H-imidazo[4,5-b]pyridin-3- yl)benzyl)pyrrolidin-3-yl)-3-formyl-4- hydroxybenzamide |
| 60 | 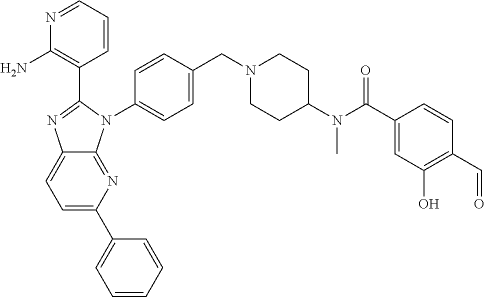 [SMILES: CN(C(=O)c1ccc(C=O)c(O)c1)C1CCN(Cc2ccc(-n3c(-c4cccnc4N)nc4ccc(-c5ccccc5)nc43)cc2)CC1] | N-(1-(4-(2-(2-aminopyridin-3-yl)-5- phenyl-3H-imidazo[4,5-b]pyridin-3- yl)benzyl)piperidin-4-yl)-4-formyl-3- hydroxy-N-methylbenzamide |
| 61 | 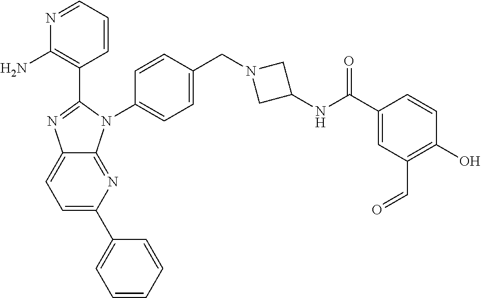 [SMILES: Nc1ncccc1-c1nc2ccc(-c3ccccc3)nc2n1-c1ccc(CN2CC(NC(=O)c3ccc(O)c(C=O)c3)C2)cc1] | N-(1-(4-(2-(2-aminopyridin-3-yl)-5- phenyl-3H-imidazo[4,5-b]pyridin-3- yl)benzyl)azetidin-3-yl)-3-formyl-4- hydroxybenzamide |
| 62 | 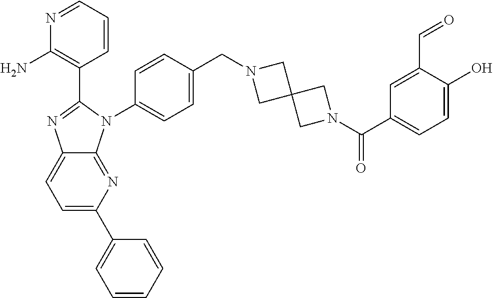 [SMILES: Nc1ncccc1-c1nc2ccc(-c3ccccc3)nc2n1-c1ccc(CN2CC3(C2)CN(C(=O)c2ccc(O)c(C=O)c2)C3)cc1] | 5-(6-(4-(2-(2-aminopyridin-3-yl)-5-phenyl- 3H-imidazo[4,5-b]pyridin-3-yl)benzyl)- 2,6-diazaspiro[3.3]heptane-2- carbonyl)-2-hydroxybenzaldehyde |
| 63 | 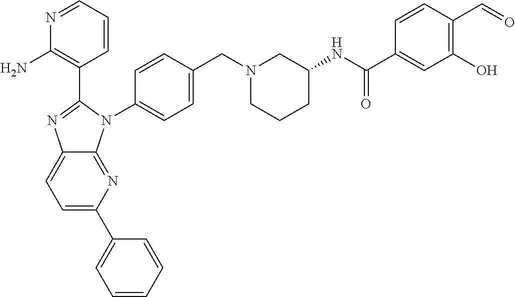 [SMILES: Nc1ncccc1-c1nc2ccc(-c3ccccc3)nc2n1-c1ccc(CN2CCC[C@@H](NC(=O)c3ccc(C=O)c(O)c3)C2)cc1] | (R)-N-(1-(4-(2-(2-aminopyridin-3-yl)-5- phenyl-3H-imidazo[4,5-b]pyridin-3- yl)benzyl)piperidin-3-yl)-4-formyl-3- hydroxybenzamide |
| 64 | 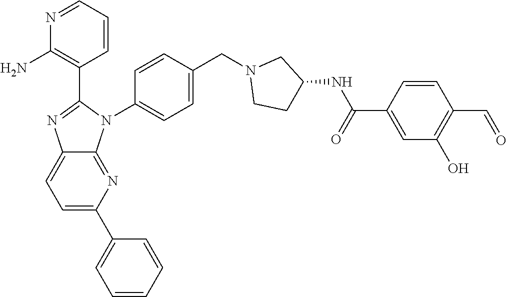 [SMILES: Nc1ncccc1-c1nc2ccc(-c3ccccc3)nc2n1-c1ccc(CN2CC[C@@H](NC(=O)c3ccc(C=O)c(O)c3)C2)cc1] | (R)-N-(1-(4-(2-(2-aminopyridin-3-yl)-5- phenyl-3H-imidazo[4,5-b]pyridin-3- yl)benzyl)pyrrolidin-3-yl)-4-formyl-3- hydroxybenzamide |
| 65 | 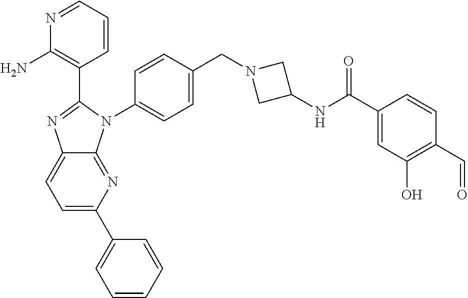 [SMILES: Nc1ncccc1-c1nc2ccc(-c3ccccc3)nc2n1-c1ccc(CN2CC(NC(=O)c3ccc(C=O)c(O)c3)C2)cc1] | N-(1-(4-(2-(2-aminopyridin-3-yl)-5- phenyl-3H-imidazo[4,5-b]pyridin-3- yl)benzyl)azetidin-3-yl)-4-formyl-3- hydroxybenzamide |
| 66 | 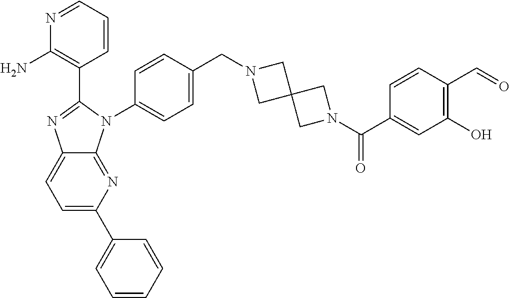 [SMILES: Nc1ncccc1-c1nc2ccc(-c3ccccc3)nc2n1-c1ccc(CN2CC3(C2)CN(C(=O)c2ccc(C=O)c(O)c2)C3)cc1] | 4-(6-(4-(2-(2-aminopyridin-3-yl)-5- phenyl-3H-imidazo[4,5-b]pyridin-3- yl)benzyl)-2,6-diazaspiro[3.3]heptane-2- carbonyl)-2-hydroxybenzaldehyde |
| 67 | 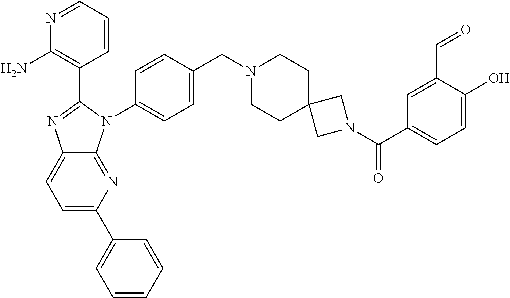 [SMILES: Nc1ncccc1-c1nc2ccc(-c3ccccc3)nc2n1-c1ccc(CN2CCC3(CC2)CN(C(=O)c2ccc(O)c(C=O)c2)C3)cc1] | 5-(7-(4-(2-(2-aminopyridin-3-yl)-5- phenyl-3H-imidazo[4,5-b]pyridin-3- yl)benzyl)-2,7-diazaspiro[3.5]nonane-2- carbonyl)-2-hydroxybenzaldehyde |
| 68 | 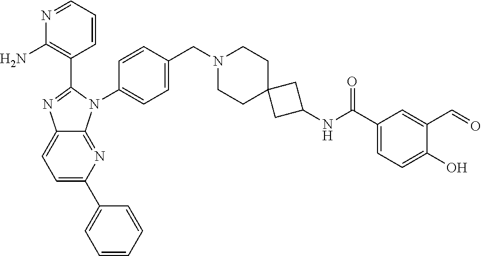 [SMILES: Nc1ncccc1-c1nc2ccc(-c3ccccc3)nc2n1-c1ccc(CN2CCC3(CC2)CC(NC(=O)c2ccc(O)c(C=O)c2)C3)cc1] | N-(7-(4-(2-(2-aminopyridin-3-yl)-5- phenyl-3H-imidazo[4,5-b]pyridin-3- yl)benzyl)-7-azaspiro[3.5]nonan-2-yl)- 3-formy1-4-hydroxybenzamide |
| 69 | 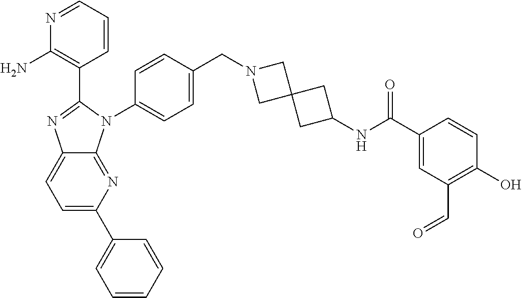 [SMILES: Nc1ncccc1-c1nc2ccc(-c3ccccc3)nc2n1-c1ccc(CN2CC3(CC(NC(=O)c4ccc(O)c(C=O)c4)C3)C2)cc1] | N-(2-(4-(2-(2-aminopyridin-3-yl)-5-phenyl- 3H-imidazo[4,5-b]pyridin-3-yl)benzyl)-2- azaspiro[3.3]heptan-6-yl)-3-formyl-4- hydroxybenzamide |
| 70 | 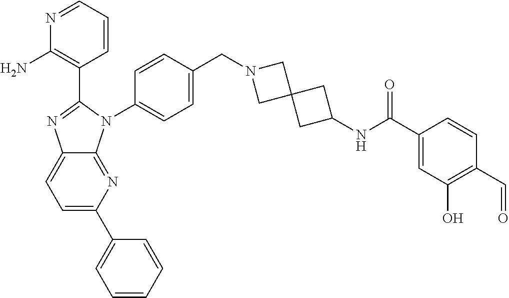 [SMILES: Nc1ncccc1-c1nc2ccc(-c3ccccc3)nc2n1-c1ccc(CN2CC3(CC(NC(=O)c4ccc(C=O)c(O)c4)C3)C2)cc1] | N-(2-(4-(2-(2-aminopyridin-3-yl)-5-phenyl- 3H-imidazo[4,5-b]pyridin-3-yl)benzyl)-2- azaspiro[3.3]heptan-6-yl)-4-formyl-3- hydroxybenzamide |
| 71 | 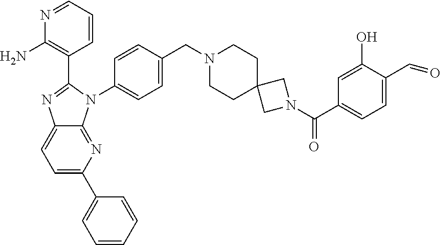 [SMILES: Nc1ncccc1-c1nc2ccc(-c3ccccc3)nc2n1-c1ccc(CN2CCC3(CC2)CN(C(=O)c2ccc(C=O)c(O)c2)C3)cc1] | 4-(7-(4-(2-(2-aminopyridin-3-yl)-5-phenyl- 3H-imidazo[4,5-b]pyridin-3-yl)benzyl)-2,7- diazaspiro[3.5]nonane-2-carbonyl)-2- hydroxybenzaldehyde |
| 72 | 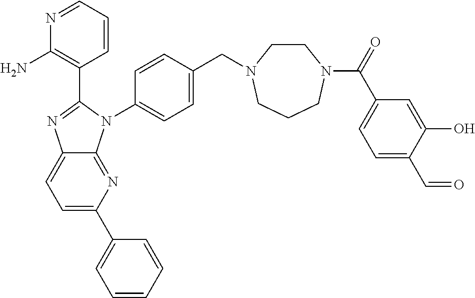 [SMILES: Nc1ncccc1-c1nc2ccc(-c3ccccc3)nc2n1-c1ccc(CN2CCCN(C(=O)c3ccc(C=O)c(O)c3)CC2)cc1] | 4-(4-(4-(2-(2-aminopyridin-3-yl)-5-phenyl- 3H-imidazo[4,5-b]pyridin-3-yl)benzyl)-1,4- diazepane-1-carbonyl)-2- hydroxybenzaldehyde |
| 73 | 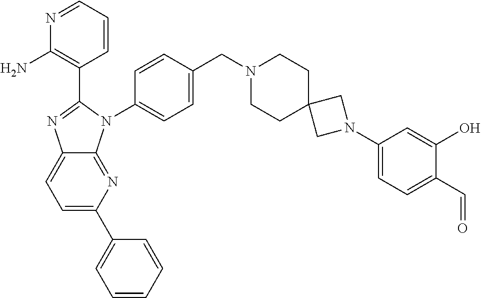 [SMILES: Nc1ncccc1-c1nc2ccc(-c3ccccc3)nc2n1-c1ccc(CN2CCC3(CC2)CN(c2ccc(C=O)c(O)c2)C3)cc1] | 4-(7-(4-(2-(2-aminopyridin-3-yl)-5-phenyl- 3H-imidazo[4,5-b]pyridin-3-yl)benzyl)-2,7- diazaspiro[3.5]nonan-2-yl)-2- hydroxybenzaldehyde |
| 74 | 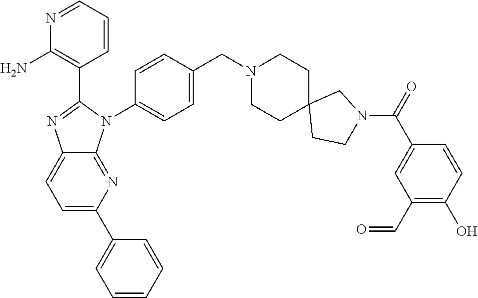 [SMILES: Nc1ncccc1-c1nc2ccc(-c3ccccc3)nc2n1-c1ccc(CN2CCC3(CC2)CCN(C(=O)c2ccc(O)c(C=O)c2)C3)cc1] | 5-(8-(4-(2-(2-aminopyridin-3-yl)-5-phenyl- 3H-imidazo[4,5-b]pyridin-3-yl)benzyl)-2,8- diazaspiro[4.5]decane-2-carbonyl)-2- hydroxybenzaldehyde |
| 75 | 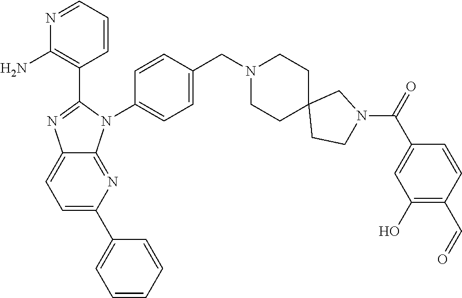 [SMILES: Nc1ncccc1-c1nc2ccc(-c3ccccc3)nc2n1-c1ccc(CN2CCC3(CC2)CCN(C(=O)c2ccc(C=O)c(O)c2)C3)cc1] | 4-(8-(4-(2-(2-aminopyridin-3-yl)-5-phenyl- 3H-imidazo[4,5-b]pyridin-3-yl)benzyl)-2,8- diazaspiro[4.5]decane-2-carbonyl)-2- hydroxybenzaldehyde |
| 76 | 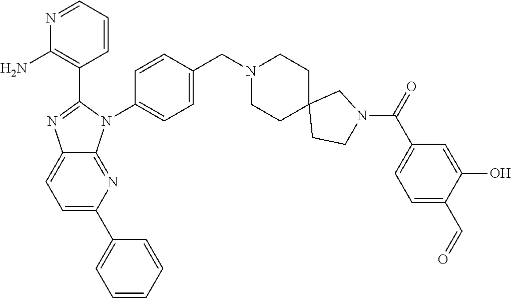 [SMILES: Nc1ncccc1-c1nc2ccc(-c3ccccc3)nc2n1-c1ccc(CN2CCC3(CC2)CCN(C(=O)c2ccc(C=O)c(O)c2)C3)cc1] | 4-(8-(4-(2-(2-aminopyridin-3-yl)-5-phenyl- 3H-imidazo[4,5-b]pyridin-3-yl)benzyl)-2,8- diazaspiro[4.5]decan-2-yl)-2- hydroxybenzaldehyde |
| 77 | 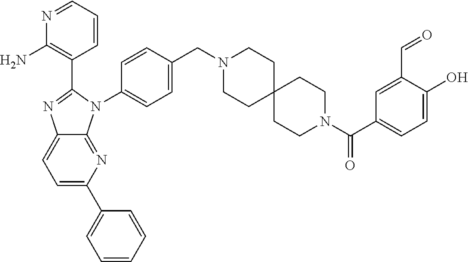 [SMILES: Nc1ncccc1-c1nc2ccc(-c3ccccc3)nc2n1-c1ccc(CN2CCC3(CC2)CCN(C(=O)c2ccc(O)c(C=O)c2)CC3)cc1] | 5-(9-(4-(2-(2-aminopyridin-3-yl)-5-phenyl- 3H-imidazo[4,5-b]pyridin-3-yl)benzyl)-3,9- diazaspiro[5.5]undecane-3-carbonyl)-2- hydroxybenzaldehyde |
| 78 | 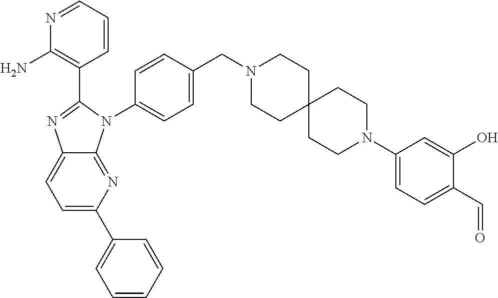 [SMILES: Nc1ncccc1-c1nc2ccc(-c3ccccc3)nc2n1-c1ccc(CN2CCC3(CC2)CCN(c2ccc(C=O)c(O)c2)CC3)cc1] | 4-(9-(4-(2-(2-aminopyridin-3-yl)-5-phenyl- 3H-imidazo[4,5-b]pyridin-3-yl)benzyl)-3,9- diazaspiro[5.5]undecan-3-yl)-2- hydroxybenzaldehyde |
| 79 | 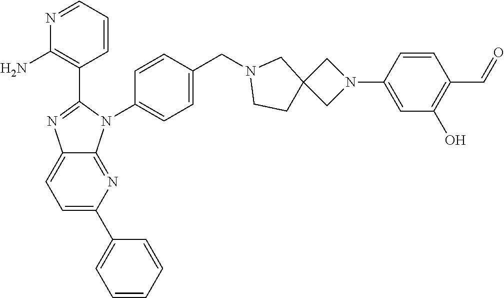 [SMILES: Nc1ncccc1-c1nc2ccc(-c3ccccc3)nc2n1-c1ccc(CN2CCC3(C2)CN(c2ccc(C=O)c(O)c2)C3)cc1] | 4-(6-(4-(2-(2-aminopyridin-3-yl)-5-phenyl- 3H-imidazo[4,5-b]pyridin-3-yl)benzyl)-2,6- diazaspiro[3.4]octan-2-yl)-2- hydroxybenzaldehyde |
| 80 | 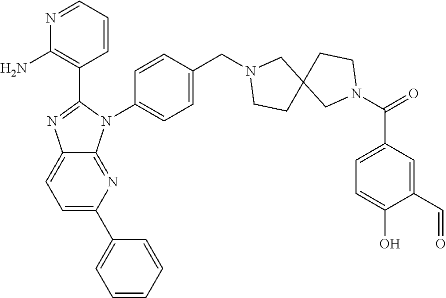 [SMILES: Nc1ncccc1-c1nc2ccc(-c3ccccc3)nc2n1-c1ccc(CN2CCC3(CCN(C(=O)c4ccc(O)c(C=O)c4)C3)C2)cc1] | 5-(7-(4-(2-(2-aminopyridin-3-yl)-5-phenyl- 3H-imidazo[4,5-b]pyridin-3-yl)benzyl)-2,7- diazaspiro[4.4]nonane-2-carbonyl)-2- hydroxybenzaldehyde |
| 81 | 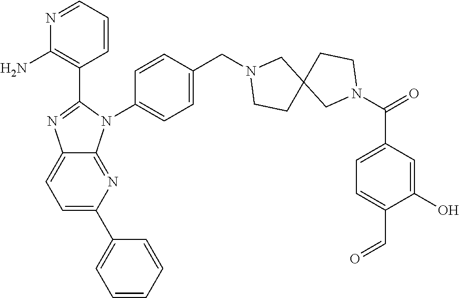 [SMILES: Nc1ncccc1-c1nc2ccc(-c3ccccc3)nc2n1-c1ccc(CN2CCC3(CCN(C(=O)c4ccc(C=O)c(O)c4)C3)C2)cc1] | 4-(7-(4-(2-(2-aminopyridin-3-yl)-5-phenyl- 3H-imidazo[4,5-b]pyridin-3-yl)benzyl)-2,7- diazaspiro[4.4]nonane-2-carbonyl)-2- hydroxybenzaldehyde |
| 82 | 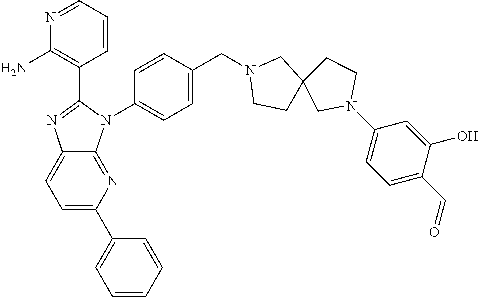 [SMILES: Nc1ncccc1-c1nc2ccc(-c3ccccc3)nc2n1-c1ccc(CN2CCC3(CCN(c4ccc(C=O)c(O)c4)C3)C2)cc1] | 4-(7-(4-(2-(2-aminopyridin-3-yl)-5-phenyl- 3H-imidazo[4,5-b]pyridin-3-yl)benzyl)-2,7- diazaspiro[4.4]nonan-2-yl)-2- hydroxybenzaldehyde |
| 83 | 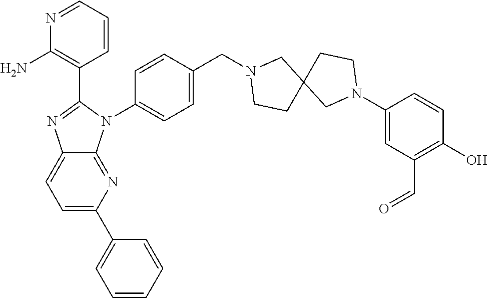 [SMILES: Nc1ncccc1-c1nc2ccc(-c3ccccc3)nc2n1-c1ccc(CN2CCC3(CCN(c4ccc(O)c(C=O)c4)C3)C2)cc1] | 5-(7-(4-(2-(2-aminopyridin-3-yl)-5-phenyl- 3H-imidazo[4,5-b]pyridin-3-yl)benzyl)-2,7- diazaspiro[4.4]nonan-2-yl)-2- hydroxybenzaldehyde |
| 84 | 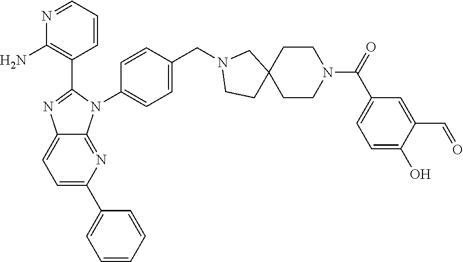 [SMILES: Nc1ncccc1-c1nc2ccc(-c3ccccc3)nc2n1-c1ccc(CN2CCC3(CCN(C(=O)c4ccc(O)c(C=O)c4)CC3)C2)cc1] | 5-(2-(4-(2-(2-aminopyridin-3-yl)-5-phenyl- 3H-imidazo[4,5-b]pyridin-3-yl)benzyl)-2,8- diazaspiro[4.5]decane-8-carbonyl)-2- hydroxybenzaldehyde |
| 85 | 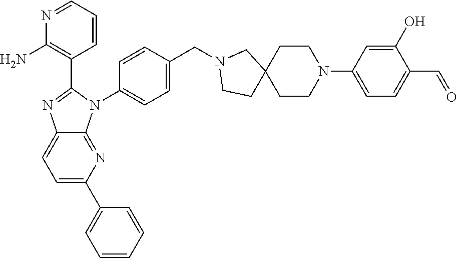 [SMILES: Nc1ncccc1-c1nc2ccc(-c3ccccc3)nc2n1-c1ccc(CN2CCC3(CCN(c4ccc(C=O)c(O)c4)CC3)C2)cc1] | 4-(2-(4-(2-(2-aminopyridin-3-yl)-5-phenyl- 3H-imidazo[4,5-b]pyridin-3-yl)benzyl)-2,8- diazaspiro[4.5]decan-8-yl)-2- hydroxybenzaldehyde |
| 86 | 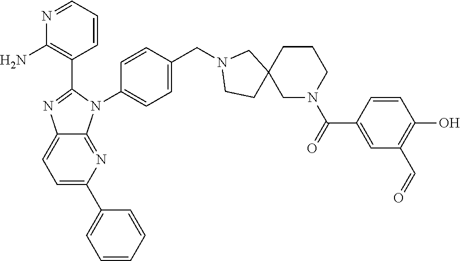 [SMILES: Nc1ncccc1-c1nc2ccc(-c3ccccc3)nc2n1-c1ccc(CN2CCC3(CCCN(C(=O)c4ccc(O)c(C=O)c4)C3)C2)cc1] | 5-(2-(4-(2-(2-aminopyridin-3-yl)-5-phenyl- 3H-imidazo[4,5-b]pyridin-3-yl)benzyl)-2,7- diazaspiro[4.5]decane-7-carbonyl)-2- hydroxybenzaldehyde |
| 87 | 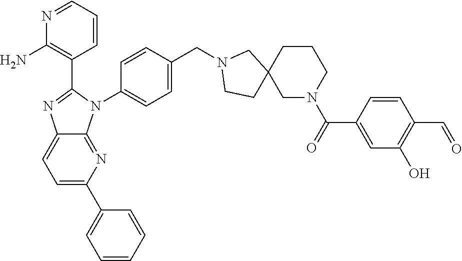 [SMILES: Nc1ncccc1-c1nc2ccc(-c3ccccc3)nc2n1-c1ccc(CN2CCC3(CCCN(C(=O)c4ccc(C=O)c(O)c4)C3)C2)cc1] | 4-(2-(4-(2-(2-aminopyridin-3-yl)-5-phenyl- 3H-imidazo[4,5-b]pyridin-3-yl)benzyl)-2,7- diazaspiro[4.5]decane-7-carbonyl)-2- hydroxybenzaldehyde |
| 88 | 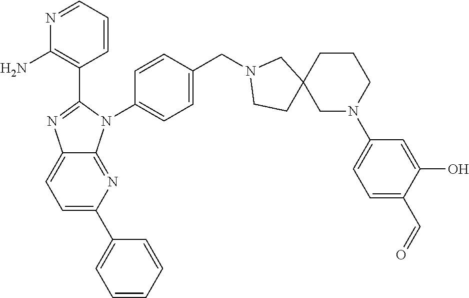 [SMILES: Nc1ncccc1-c1nc2ccc(-c3ccccc3)nc2n1-c1ccc(CN2CCC3(CCCN(c4ccc(C=O)c(O)c4)C3)C2)cc1] | 4-(2-(4-(2-(2-aminopyridin-3-yl)-5-phenyl- 3H-imidazo[4,5-b]pyridin-3-yl)benzyl)-2,7- diazaspiro[4.5]decan-7-yl)-2- hydroxybenzaldehyde |
| 89 | 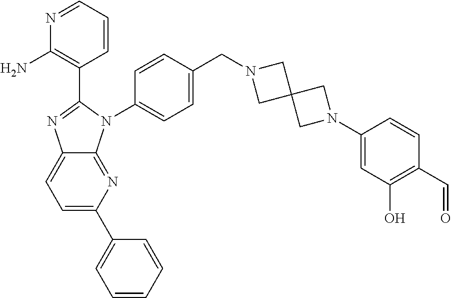 [SMILES: Nc1ncccc1-c1nc2ccc(-c3ccccc3)nc2n1-c1ccc(CN2CC3(C2)CN(c2ccc(C=O)c(O)c2)C3)cc1] | 4-(6-(4-(2-(2-aminopyridin-3-yl)-5-phenyl- 3H-imidazo[4,5-b]pyridin-3-yl)benzyl)-2,6- diazaspiro[3.3]heptan-2-yl)-2- hydroxybenzaldehyde |
| 90 | 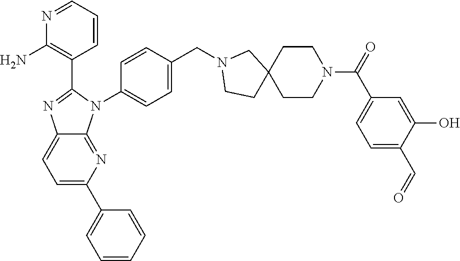 [SMILES: Nc1ncccc1-c1nc2ccc(-c3ccccc3)nc2n1-c1ccc(CN2CCC3(CCN(C(=O)c4ccc(C=O)c(O)c4)CC3)C2)cc1] | 4-(2-(4-(2-(2-aminopyridin-3-yl)-5-phenyl- 3H-imidazo[4,5-b]pyridin-3-yl)benzyl)-2,8- diazaspiro[4.5]decane-8-carbonyl)-2- hydroxybenzaldehyde |
| 91 | 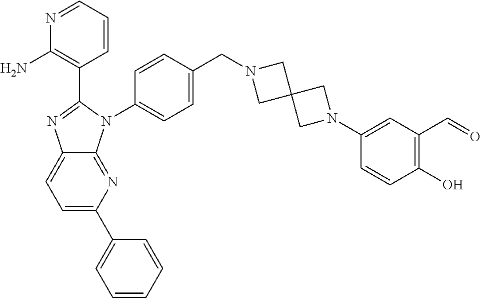 [SMILES: Nc1ncccc1-c1nc2ccc(-c3ccccc3)nc2n1-c1ccc(CN2CC3(C2)CN(c2ccc(O)c(C=O)c2)C3)cc1] | 5-(6-(4-(2-(2-aminopyridin-3-yl)-5-phenyl- 3H-imidazo[4,5-b]pyridin-3-yl)benzyl)-2,6- diazaspiro[3.3]heptan-2-yl)-2- hydroxybenzaldehyde |
| 92 | 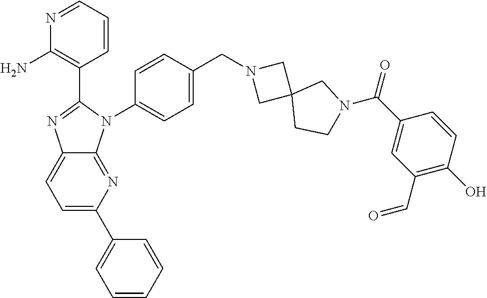 [SMILES: Nc1ncccc1-c1nc2ccc(-c3ccccc3)nc2n1-c1ccc(CN2CC3(CCN(C(=O)c4ccc(O)c(C=O)c4)C3)C2)cc1] | 5-(2-(4-(2-(2-aminopyridin-3-yl)-5-phenyl- 3H-imidazo[4,5-b]pyridin-3-yl)benzyl)-2,6- diazaspiro[3.4]octane-6-carbonyl)-2- hydroxybenzaldehyde |
| 93 | 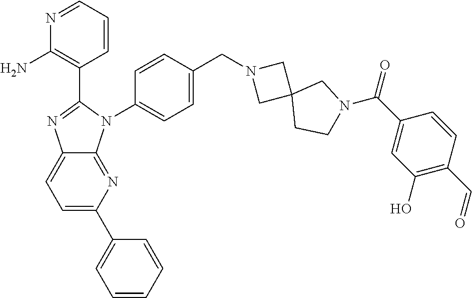 [SMILES: Nc1ncccc1-c1nc2ccc(-c3ccccc3)nc2n1-c1ccc(CN2CC3(CCN(C(=O)c4ccc(C=O)c(O)c4)C3)C2)cc1] | 4-(2-(4-(2-(2-aminopyridin-3-yl)-5-phenyl- 3H-imidazo[4,5-b]pyridin-3-yl)benzyl)-2,6- diazaspiro[3.4]octane-6-carbonyl)-2- hydroxybenzaldehyde |
| 94 | 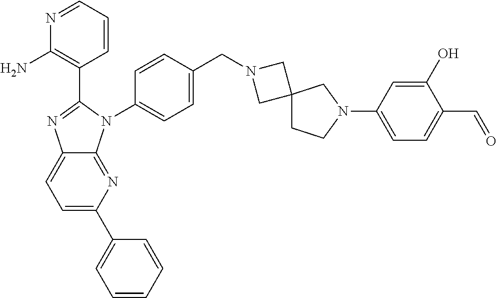 [SMILES: Nc1ncccc1-c1nc2ccc(-c3ccccc3)nc2n1-c1ccc(CN2CC3(CCN(c4ccc(C=O)c(O)c4)C3)C2)cc1] | 4-(2-(4-(2-(2-aminopyridin-3-yl)-5-phenyl- 3H-imidazo[4,5-b]pyridin-3-yl)benzyl)-2,6- diazaspiro[3.4]octan-6-yl)-2- hydroxybenzaldehyde |
| 95 | 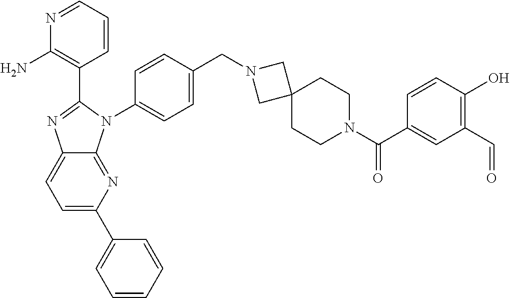 [SMILES: Nc1ncccc1-c1nc2ccc(-c3ccccc3)nc2n1-c1ccc(CN2CC3(CCN(C(=O)c4ccc(O)c(C=O)c4)CC3)C2)cc1] | 5-(2-(4-(2-(2-aminopyridin-3-yl)-5-phenyl- 3H-imidazo[4,5-b]pyridin-3-yl)benzyl)-2,7- diazaspiro[3.5]nonane-7-carbonyl)-2- hydroxybenzaldehyde |
| 96 | 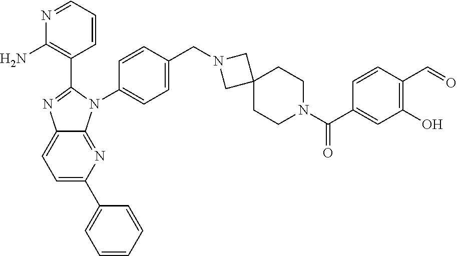 [SMILES: Nc1ncccc1-c1nc2ccc(-c3ccccc3)nc2n1-c1ccc(CN2CC3(CCN(C(=O)c4ccc(C=O)c(O)c4)CC3)C2)cc1] | 4-(2-(4-(2-(2-aminopyridin-3-yl)-5-phenyl- 3H-imidazo[4,5-b]pyridin-3-yl)benzyl)-2,7- diazaspiro[3.5]nonane-7-carbonyl)-2- hydroxybenzaldehyde |
| 97 | 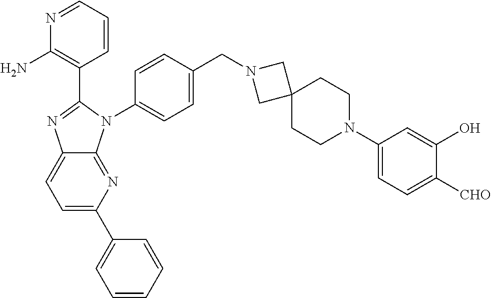 [SMILES: Nc1ncccc1-c1nc2ccc(-c3ccccc3)nc2n1-c1ccc(CN2CC3(CCN(c4ccc(C=O)c(O)c4)CC3)C2)cc1] | 4-(2-(4-(2-(2-aminopyridin-3-yl)-5-phenyl- 3H-imidazo[4,5-b]pyridin-3-yl)benzyl)-2,7- diazaspiro[3.5]nonan-7-yl)-2- hydroxybenzaldehyde |
| 98 | 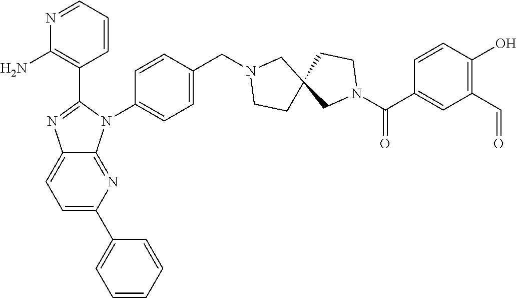 [SMILES: Nc1ncccc1-c1nc2ccc(-c3ccccc3)nc2n1-c1ccc(CN2CC[C@@]3(CCN(C(=O)c4ccc(O)c(C=O)c4)C3)C2)cc1] | (R)-5-(7-(4-(2-(2-aminopyridin-3-yl)-5- phenyl-3H-imidazo[4,5-b]pyridin-3- yl)benzyl)-2,7-diazaspiro[4.4]nonane-2- carbonyl)-2-hydroxybenzaldehyde |
| 99 | 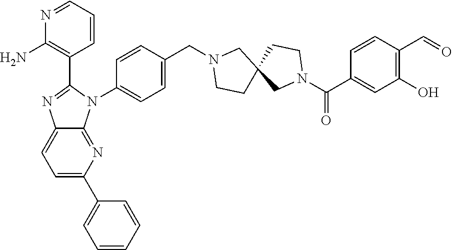 [SMILES: Nc1ncccc1-c1nc2ccc(-c3ccccc3)nc2n1-c1ccc(CN2CC[C@@]3(CCN(C(=O)c4ccc(C=O)c(O)c4)C3)C2)cc1] | (R)-4-(7-(4-(2-(2-aminopyridin-3-yl)-5- phenyl-3H-imidazo[4,5-b]pyridin-3- yl)benzyl)-2,7-diazaspiro[4.4]nonane-2- carbonyl)-2-hydroxybenzaldehyde |
| 100 | 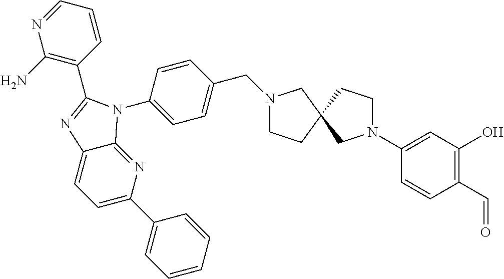 [SMILES: Nc1ncccc1-c1nc2ccc(-c3ccccc3)nc2n1-c1ccc(CN2CC[C@@]3(CCN(c4ccc(C=O)c(O)c4)C3)C2)cc1] | (R)-4-(7-(4-(2-(2-aminopyridin-3-yl)-5- phenyl-3H-imidazo[4,5-b]pyridin-3- yl)benzyl)-2,7-diazaspiro[4.4]nonan- 2-yl)-2-hydroxybenzaldehyde |
| 101 | 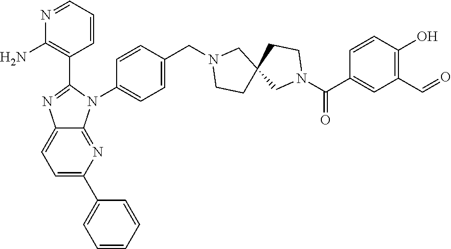 [SMILES: Nc1ncccc1-c1nc2ccc(-c3ccccc3)nc2n1-c1ccc(CN2CC[C@]3(CCN(C(=O)c4ccc(O)c(C=O)c4)C3)C2)cc1] | (S)-5-(7-(4-(2-(2-aminopyridin-3-yl)-5- phenyl-3H-imidazo[4,5-b]pyridin-3- yl)benzyl)-2,7-diazaspiro[4.4]nonane-2- carbonyl)-2-hydroxybenzaldehyde |
| 102 | 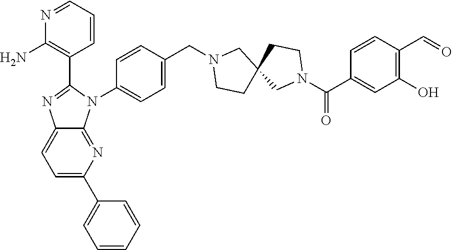 [SMILES: Nc1ncccc1-c1nc2ccc(-c3ccccc3)nc2n1-c1ccc(CN2CC[C@]3(CCN(C(=O)c4ccc(C=O)c(O)c4)C3)C2)cc1] | (S)-4-(7-(4-(2-(2-aminopyridin-3-yl)-5- phenyl-3H-imidazo[4,5-b]pyridin-3- yl)benzyl)-2,7-diazaspiro[4.4]nonane-2- carbonyl)-2-hydroxybenzaldehyde |
| 103 | 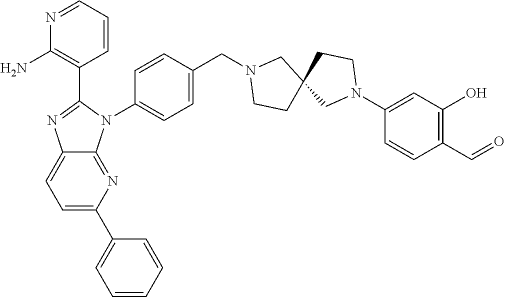 [SMILES: Nc1ncccc1-c1nc2ccc(-c3ccccc3)nc2n1-c1ccc(CN2CC[C@]3(CCN(c4ccc(C=O)c(O)c4)C3)C2)cc1] | (S)-4-(7-(4-(2-(2-aminopyridin-3-yl)-5- phenyl-3H-imidazo[4,5-b]pyridin-3- yl)benzyl)-2,7-diazaspiro[4.4]nonan- 2-yl)-2-hydroxybenzaldehyde |
| 104 | 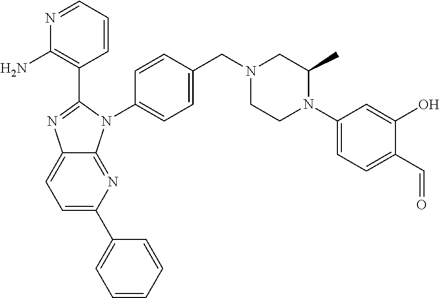 [SMILES: C[C@@H]1CN(Cc2ccc(-n3c(-c4cccnc4N)nc4ccc(-c5ccccc5)nc43)cc2)CCN1c1ccc(C=O)c(O)c1] | (R)-4-(4-(4-(2-(2-aminopyridin-3-yl)-5- phenyl-3H-imidazo[4,5-b]pyridin-3- yl)benzyl)-2-methylpiperazin-1-yl)-2- hydroxybenzaldehyde |
| 105 | 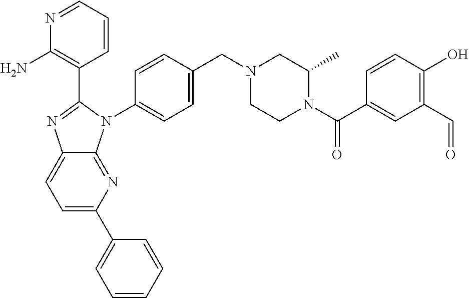 [SMILES: C[C@H]1CN(Cc2ccc(-n3c(-c4cccnc4N)nc4ccc(-c5ccccc5)nc43)cc2)CCN1C(=O)c1ccc(O)c(C=O)c1] | (S)-5-(4-(4-(2-(2-aminopyridin-3-yl)-5- phenyl-3H-imidazo[4,5-b]pyridin-3- yl)benzyl)-2-methylpiperazine-1-carbonyl)- 2-hydroxybenzaldehyde |
| 106 | 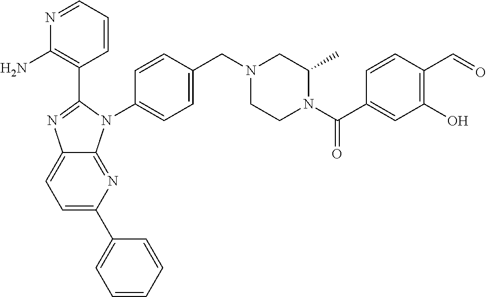 [SMILES: C[C@H]1CN(Cc2ccc(-n3c(-c4cccnc4N)nc4ccc(-c5ccccc5)nc43)cc2)CCN1C(=O)c1ccc(C=O)c(O)c1] | (S)-4-(4-(4-(2-(2-aminopyridin-3-yl)-5- phenyl-3H-imidazo[4,5-b]pyridin-3- yl)benzyl)-2-methylpiperazine-1-carbonyl)- 2-hydroxybenzaldehyde |
| 107 | 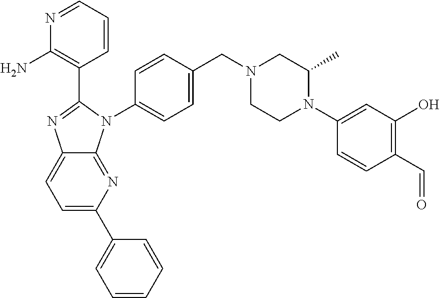 [SMILES: C[C@H]1CN(Cc2ccc(-n3c(-c4cccnc4N)nc4ccc(-c5ccccc5)nc43)cc2)CCN1c1ccc(C=O)c(O)c1] | (S)-4-(4-(4-(2-(2-aminopyridin-3-yl)-5- phenyl-3H-imidazo[4,5-b]pyridin-3- yl)benzyl)-2-methylpiperazin-1-yl)-2- hydroxybenzaldehyde |
| 108 | 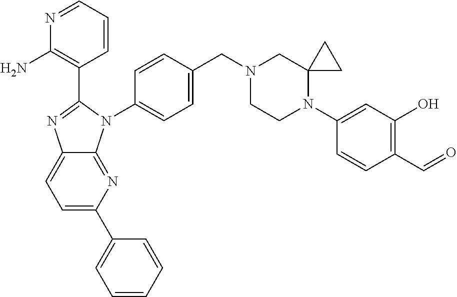 [SMILES: Nc1ncccc1-c1nc2ccc(-c3ccccc3)nc2n1-c1ccc(CN2CCN(c3ccc(C=O)c(O)c3)C3(CC3)C2)cc1] | 4-(7-(4-(2-(2-aminopyridin-3-yl)-5-phenyl- 3H-imidazo[4,5-b]pyridin-3-yl)benzyl)-4,7- diazaspiro[2.5]octan-4-yl)-2- hydroxybenzaldehyde |
| 109 | 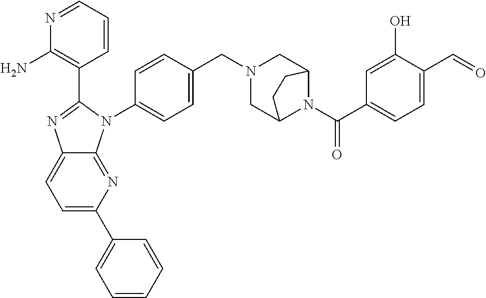 [SMILES: Nc1ncccc1-c1nc2ccc(-c3ccccc3)nc2n1-c1ccc(CN2CC3CCC(C2)N3C(=O)c2ccc(C=O)c(O)c2)cc1] | 4-(3-(4-(2-(2-aminopyridin-3-yl)-5-phenyl- 3H-imidazo[4,5-b]pyridin-3-yl)benzyl)-3,8- diazabicyclo[3.2.1]octane-8-carbonyl)-2- hydroxybenzaldehyde |
| 110 | 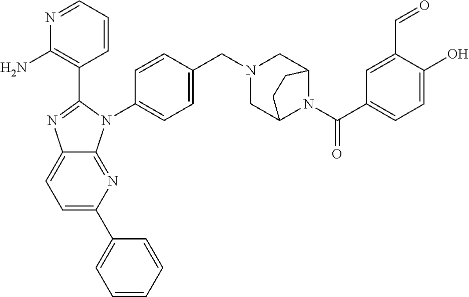 [SMILES: Nc1ncccc1-c1nc2ccc(-c3ccccc3)nc2n1-c1ccc(CN2CC3CCC(C2)N3C(=O)c2ccc(O)c(C=O)c2)cc1] | 5-(3-(4-(2-(2-aminopyridin-3-yl)-5-phenyl- 3H-imidazo[4,5-b]pyridin-3-yl)benzyl)-3,8- diazabicyclo[3.2.1]octane-8-carbonyl)-2- hydroxybenzaldehyde |
| 111 | 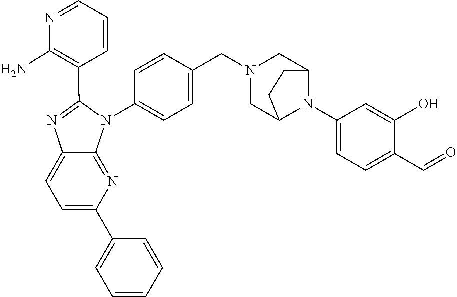 [SMILES: Nc1ncccc1-c1nc2ccc(-c3ccccc3)nc2n1-c1ccc(CN2CC3CCC(C2)N3c2ccc(C=O)c(O)c2)cc1] | 4-(3-(4-(2-(2-aminopyridin-3-yl)-5-phenyl- 3H-imidazo[4,5-b]pyridin-3-yl)benzyl)-3,8- diazabicyclo[3.2.1]octan-8-yl)-2- hydroxybenzaldehyde |
| 112 | 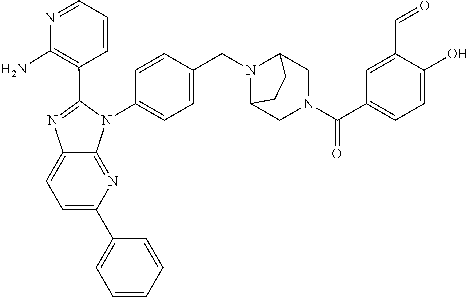 [SMILES: Nc1ncccc1-c1nc2ccc(-c3ccccc3)nc2n1-c1ccc(CN2C3CCC2CN(C(=O)c2ccc(O)c(C=O)c2)C3)cc1] | 5-(8-(4-(2-(2-aminopyridin-3-yl)-5-phenyl- 3H-imidazo[4,5-b]pyridin-3-yl)benzyl)-3,8- diazabicyclo[3.2.1]octane-3-carbonyl)-2- hydroxybenzaldehyde |
| 113 | 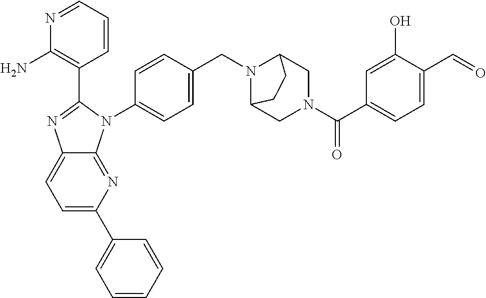 [SMILES: Nc1ncccc1-c1nc2ccc(-c3ccccc3)nc2n1-c1ccc(CN2C3CCC2CN(C(=O)c2ccc(C=O)c(O)c2)C3)cc1] | 4-(8-(4-(2-(2-aminopyridin-3-yl)-5-phenyl- 3H-imidazo[4,5-b]pyridin-3-yl)benzyl)-3,8- diazabicyclo[3.2.1]octane-3-carbonyl)-2- hydroxybenzaldehyde |
| 114 | 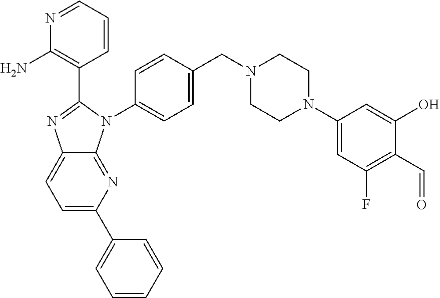 [SMILES: Nc1ncccc1-c1nc2ccc(-c3ccccc3)nc2n1-c1ccc(CN2CCN(c3cc(O)c(C=O)c(F)c3)CC2)cc1] | 4-(8-(4-(2-(2-aminopyridin-3-yl)-5-phenyl- 3H-imidazo[4,5-b]pyridin-3-yl)benzyl)-3,8- diazabicyclo[3.2.1]octan-3-yl)-2- hydroxybenzaldehyde |
| 115 | 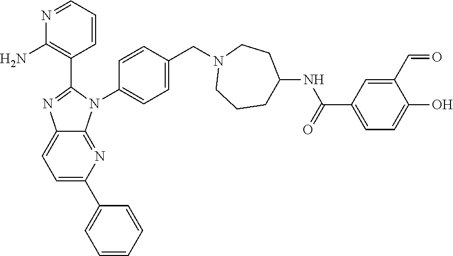 [SMILES: Nc1ncccc1-c1nc2ccc(-c3ccccc3)nc2n1-c1ccc(CN2CCCC(NC(=O)c3ccc(O)c(C=O)c3)CC2)cc1] | N-(1-(4-(2-(2-aminopyridin-3-yl)-5-phenyl- 3H-imidazo[4,5-b]pyridin-3- yl)benzyl)azepan-4-yl)-3-formyl-4- hydroxybenzamide |
| 116 | 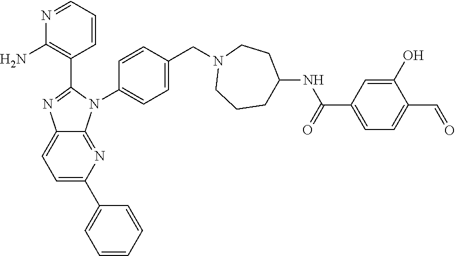 [SMILES: Nc1ncccc1-c1nc2ccc(-c3ccccc3)nc2n1-c1ccc(CN2CCCC(NC(=O)c3ccc(C=O)c(O)c3)CC2)cc1] | N-(1-(4-(2-(2-aminopyridin-3-yl)-5-phenyl- 3H-imidazo[4,5-b]pyridin-3- yl)benzyl)azepan-4-yl)-4-formyl-3- hydroxybenzamide |
| 117 | 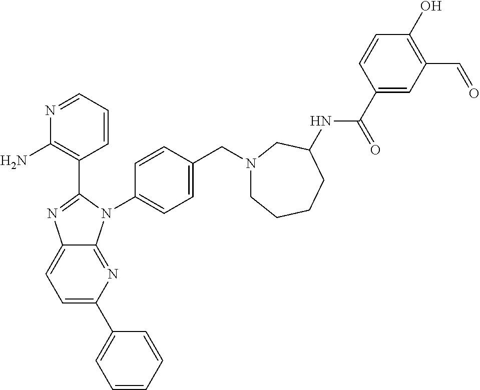 [SMILES: Nc1ncccc1-c1nc2ccc(-c3ccccc3)nc2n1-c1ccc(CN2CCCCC(NC(=O)c3ccc(O)c(C=O)c3)C2)cc1] | N-(1-(4-(2-(2-aminopyridin-3-yl)-5-phenyl- 3H-imidazo[4,5-b]pyridin-3- yl)benzyl)azepan-3-yl)-3-formyl-4- hydroxybenzamide |
| 118 | 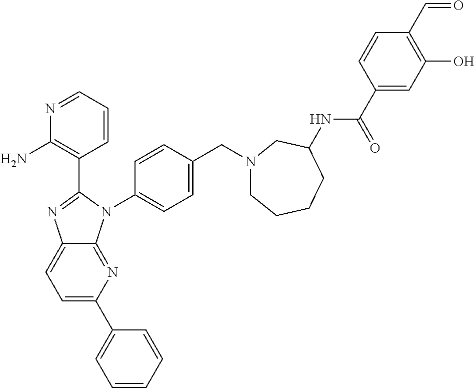 [SMILES: Nc1ncccc1-c1nc2ccc(-c3ccccc3)nc2n1-c1ccc(CN2CCCCC(NC(=O)c3ccc(C=O)c(O)c3)C2)cc1] | N-(1-(4-(2-(2-aminopyridin-3-yl)-5-phenyl- 3H-imidazo[4,5-b]pyridin-3- yl)benzyl)azepan-3-yl)-4-formyl-3- hydroxybenzamide |
| 119 | 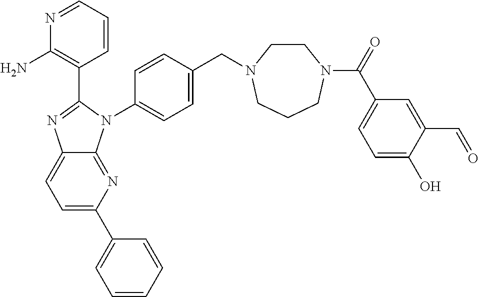 [SMILES: Nc1ncccc1-c1nc2ccc(-c3ccccc3)nc2n1-c1ccc(CN2CCCN(C(=O)c3ccc(O)c(C=O)c3)CC2)cc1] | 5-(4-(4-(2-(2-aminopyridin-3-yl)-5-phenyl- 3H-imidazo[4,5-b]pyridin-3-yl)benzyl)-1,4- diazepane-1-carbonyl)-2- hydroxybenzaldehyde |
| 120 | 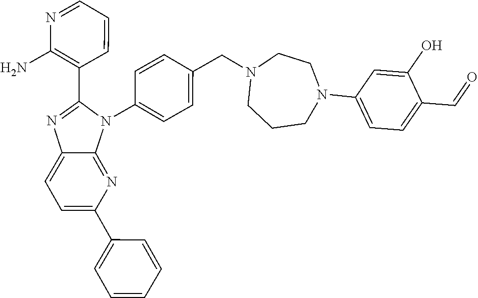 [SMILES: Nc1ncccc1-c1nc2ccc(-c3ccccc3)nc2n1-c1ccc(CN2CCCN(c3ccc(C=O)c(O)c3)CC2)cc1] | 4-(4-(4-(2-(2-aminopyridin-3-yl)-5-phenyl- 3H-imidazo[4,5-b]pyridin-3-yl)benzyl)-1,4- diazepan-1-yl)-2-hydroxybenzaldehyde |
| 121 | 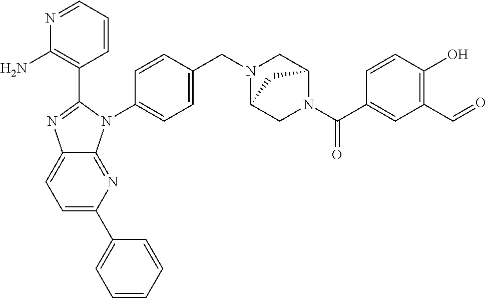 [SMILES: Nc1ncccc1-c1nc2ccc(-c3ccccc3)nc2n1-c1ccc(CN2C[C@@H]3C[C@H]2CN3C(=O)c2ccc(O)c(C=O)c2)cc1] | 5-((1S,4S)-5-(4-(2-(2-aminopyridin-3-yl)- 5-phenyl-3H-imidazo[4,5-b]pyridin-3- yl)benzyl)-2,5-diazabicyclo[2.2.1]heptane- 2-carbonyl)-2-hydroxybenzaldehyde |
| 122 | 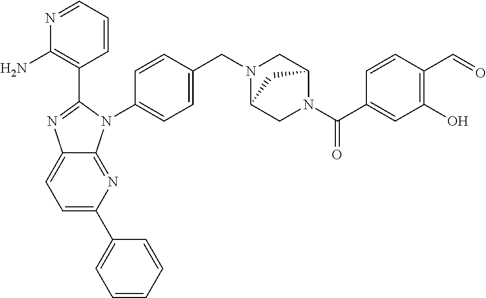 [SMILES: Nc1ncccc1-c1nc2ccc(-c3ccccc3)nc2n1-c1ccc(CN2C[C@@H]3C[C@H]2CN3C(=O)c2ccc(C=O)c(O)c2)cc1] | 4-((1S,4S)-5-(4-(2-(2-aminopyridin-3-yl)- 5-phenyl-3H-imidazo[4,5-b]pyridin-3- yl)benzyl)-2,5-diazabicyclo[2.2.1]heptane- 2-carbonyl)-2-hydroxybenzaldehyde |
| 123 | 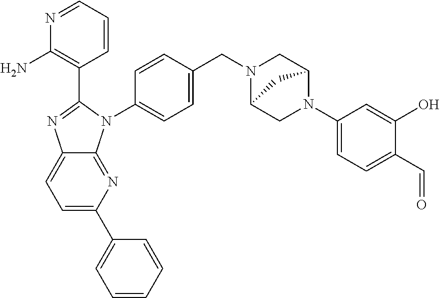 [SMILES: Nc1ncccc1-c1nc2ccc(-c3ccccc3)nc2n1-c1ccc(CN2C[C@@H]3C[C@H]2CN3c2ccc(C=O)c(O)c2)cc1] | 4-((1S,4S)-5-(4-(2-(2-aminopyridin-3-yl)-5- phenyl-3H-imidazo[4,5-b]pyridin-3- yl)benzyl)-2,5-diazabicyclo[2.2.1]heptan-2- yl)-2-hydroxybenzaldehyde |
| 124 | 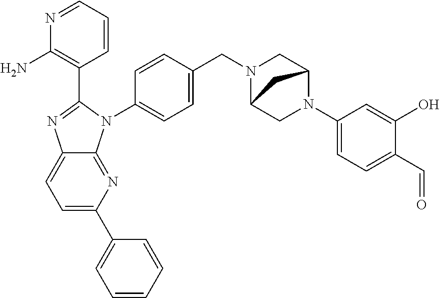 [SMILES: Nc1ncccc1-c1nc2ccc(-c3ccccc3)nc2n1-c1ccc(CN2C[C@H]3C[C@@H]2CN3c2ccc(C=O)c(O)c2)cc1] | 4-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptan-2- yl)-2-hydroxybenzaldehyde |
| 125 | 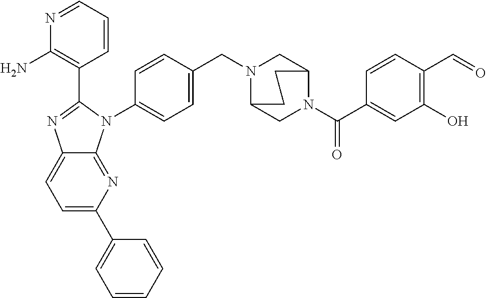 [SMILES: Nc1ncccc1-c1nc2ccc(-c3ccccc3)nc2n1-c1ccc(CN2CC3CCC2CN3C(=O)c2ccc(C=O)c(O)c2)cc1] | 4-(5-(4-(2-(2-aminopyridin-3-yl)-5-phenyl- 3H-imidazo[4,5-b]pyridin-3-yl)benzyl)-2,5- diazabicyclo[2.2.2]octane-2-carbonyl)-2- hydroxybenzaldehyde |
| 126 | 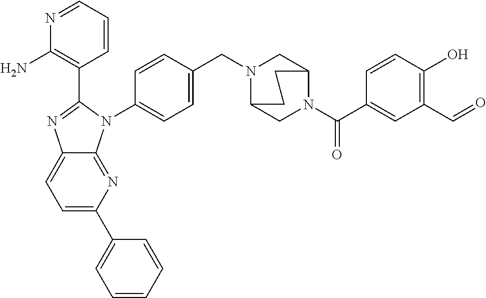 [SMILES: Nc1ncccc1-c1nc2ccc(-c3ccccc3)nc2n1-c1ccc(CN2CC3CCC2CN3C(=O)c2ccc(O)c(C=O)c2)cc1] | 5-(5-(4-(2-(2-aminopyridin-3-yl)-5-phenyl- 3H-imidazo[4,5-b]pyridin-3-yl)benzyl)-2,5- diazabicyclo[2.2.2]octane-2-carbonyl)-2- hydroxybenzaldehyde |
| 127 | 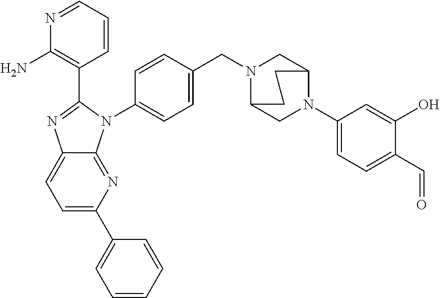 [SMILES: Nc1ncccc1-c1nc2ccc(-c3ccccc3)nc2n1-c1ccc(CN2CC3CCC2CN3c2ccc(C=O)c(O)c2)cc1] | 4-(5-(4-(2-(2-aminopyridin-3-yl)-5-phenyl- 3H-imidazo[4,5-b]pyridin-3-yl)benzyl)-2,5- diazabicyclo[2.2.2]octan-2-yl)-2- hydroxybenzaldehyde |
| 128 | 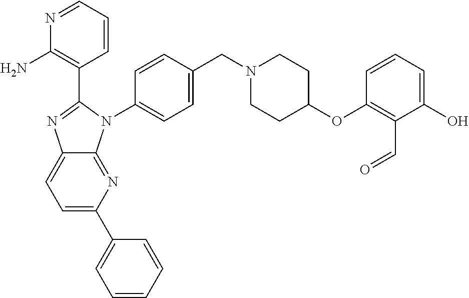 [SMILES: Nc1ncccc1-c1nc2ccc(-c3ccccc3)nc2n1-c1ccc(CN2CCC(Oc3cccc(O)c3C=O)CC2)cc1] | 2-((1-(4-(2-(2-aminopyridin-3-yl)-5-phenyl- 3H-imidazo[4,5-b]pyridin-3- yl)benzyl)piperidin-4-yl)oxy)-6- hydroxybenzaldehyde |
| 129 | 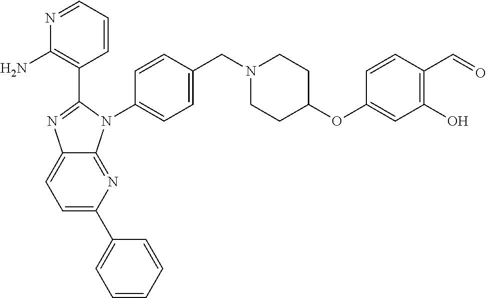 [SMILES: Nc1ncccc1-c1nc2ccc(-c3ccccc3)nc2n1-c1ccc(CN2CCC(Oc3ccc(C=O)c(O)c3)CC2)cc1] | 4-((1-(4-(2-(2-aminopyridin-3-yl)-5-phenyl- 3H-imidazo[4,5-b]pyridin-3- yl)benzyl)piperidin-4-yl)oxy)-2- hydroxybenzaldehyde |
| 130 | 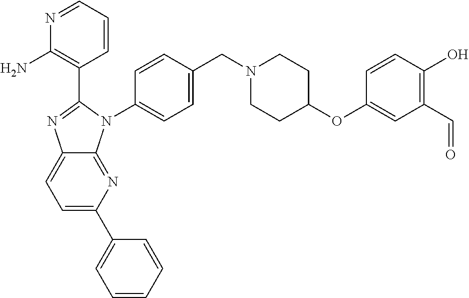 [SMILES: Nc1ncccc1-c1nc2ccc(-c3ccccc3)nc2n1-c1ccc(CN2CCC(Oc3ccc(O)c(C=O)c3)CC2)cc1] | 5-((1-(4-(2-(2-aminopyridin-3-yl)-5-phenyl- 3H-imidazo[4,5-b]pyridin-3- yl)benzyl)piperidin-4-yl)oxy)-2- hydroxybenzaldehyde |
| 131 | 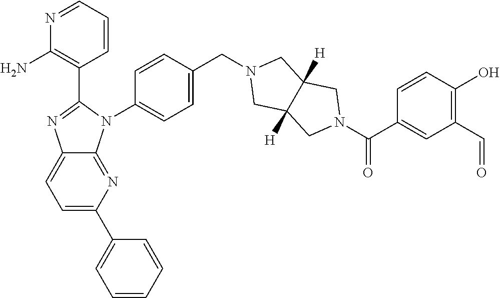 [SMILES: Nc1ncccc1-c1nc2ccc(-c3ccccc3)nc2n1-c1ccc(CN2C[C@@H]3CN(C(=O)c4ccc(O)c(C=O)c4)C[C@@H]3C2)cc1] | 5-((3aR,6aS)-5-(4-(2-(2-aminopyridin-3-yl)- 5-phenyl-3HI-imidazo[4,5-b]pyridin-3- yl)benzyl)octahydropyrrolo[3,4-c]pyrrole-2- carbonyl)-2-hydroxybenzaldehyde |
| 132 | 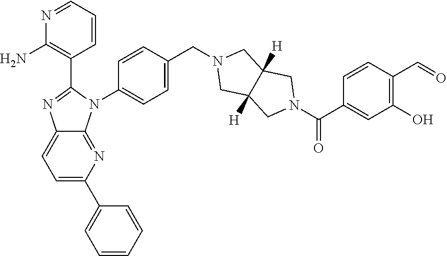 [SMILES: Nc1ncccc1-c1nc2ccc(-c3ccccc3)nc2n1-c1ccc(CN2C[C@@H]3CN(C(=O)c4ccc(C=O)c(O)c4)C[C@@H]3C2)cc1] | 4-((3aR,6aS)-5-(4-(2-(2-aminopyridin-3-yl)- 5-phenyl-3H-imidazo[4,5-b]pyridin-3- yl)benzyl)octahydropyrrolo[3,4-c]pyrrole-2- carbonyl)-2-hydroxybenzaldehyde |
| 133 | 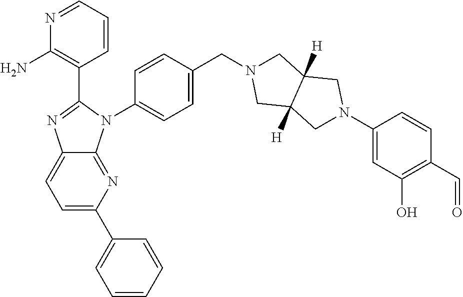 [SMILES: Nc1ncccc1-c1nc2ccc(-c3ccccc3)nc2n1-c1ccc(CN2C[C@@H]3CN(c4ccc(C=O)c(O)c4)C[C@@H]3C2)cc1] | 4-((3aR,6aS)-5-(4-(2-(2-aminopyridin-3-yl)- 5-phenyl-3H-imidazo[4,5-b]pyridin-3- yl)benzyl)hexahydropyrrolo[3,4-c]pyrrol- 2(1H)-yl)-2-hydroxybenzaldehyde |
| 134 | 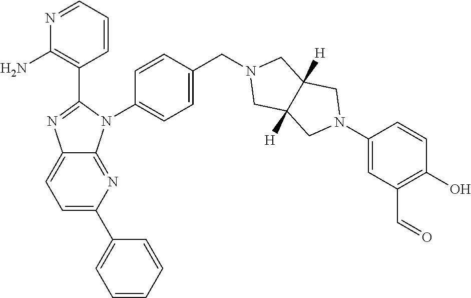 [SMILES: Nc1ncccc1-c1nc2ccc(-c3ccccc3)nc2n1-c1ccc(CN2C[C@@H]3CN(c4ccc(O)c(C=O)c4)C[C@@H]3C2)cc1] | 5-((3aR,6aS)-5-(4-(2-(2-aminopyridin-3-yl)- 5-phenyl-3H-imidazo[4,5-b]pyridin-3- yl)benzyl)hexahydropyrrolo[3,4-c]pyrrol- 2(1H)-yl)-2-hydroxybenzaldehyde |
| 135 | 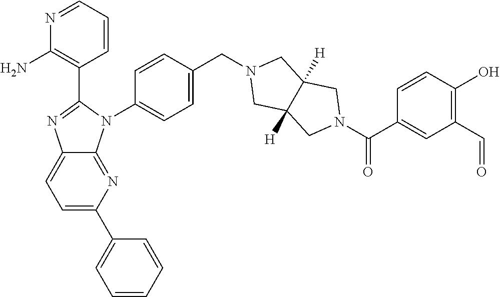 [SMILES: Nc1ncccc1-c1nc2ccc(-c3ccccc3)nc2n1-c1ccc(CN2C[C@H]3CN(C(=O)c4ccc(O)c(C=O)c4)C[C@@H]3C2)cc1] | 5-((3aS,6aS)-5-(4-(2-(2-aminopyridin-3-yl)- 5-phenyl-3H-imidazo[4,5-b]pyridin-3- yl)benzyl)octahydropyrrolo[3,4-c]pyrrole-2- carbonyl)-2-hydroxybenzaldehyde |
| 136 | 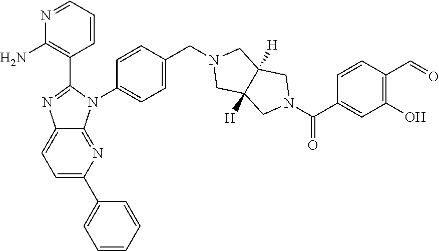 [SMILES: Nc1ncccc1-c1nc2ccc(-c3ccccc3)nc2n1-c1ccc(CN2C[C@H]3CN(C(=O)c4ccc(C=O)c(O)c4)C[C@@H]3C2)cc1] | 4-((3aS,6aS)-5-(4-(2-(2-aminopyridin-3-yl)- 5-phenyl-3H-imidazo[4,5-b]pyridin-3- yl)benzyl)octahydropyrrolo[3,4-c]pyrrole-2- carbonyl)-2-hydroxybenzaldehyde |
| 137 | 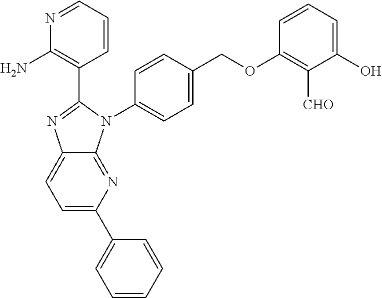 [SMILES: Nc1ncccc1-c1nc2ccc(-c3ccccc3)nc2n1-c1ccc(COc2cccc(O)c2C=O)cc1] | 2-((4-(2-(2-aminopyridin-3-yl)-5-phenyl- 3H-imidazo[4,5-b]pyridin-3- yl)benzyl)oxy)-6-hydroxybenzaldehyde |
| 138 | 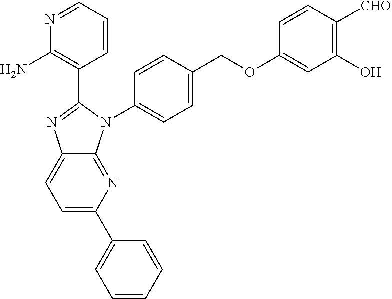 [SMILES: Nc1ncccc1-c1nc2ccc(-c3ccccc3)nc2n1-c1ccc(COc2ccc(C=O)c(O)c2)cc1] | 4-((4-(2-(2-aminopyridin-3-yl)-5-phenyl- 3H-imidazo[4,5-b]pyridin-3- yl)benzyl)oxy)-2-hydroxybenzaldehyde |
| 139 | 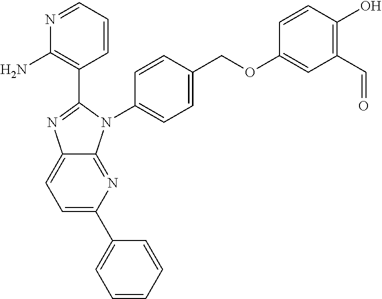 [SMILES: Nc1ncccc1-c1nc2ccc(-c3ccccc3)nc2n1-c1ccc(COc2ccc(O)c(C=O)c2)cc1] | 5-((4-(2-(2-aminopyridin-3-yl)-5-phenyl- 3H-imidazo[4,5-b]pyridin-3- yl)benzyl)oxy)-2-hydroxybenzaldehyde |
| 140 | 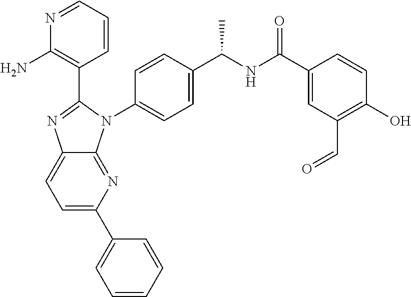 [SMILES: C[C@H](NC(=O)c1ccc(O)c(C=O)c1)c1ccc(-n2c(-c3cccnc3N)nc3ccc(-c4ccccc4)nc32)cc1] | (S)-N-(1-(4-(2-(2-aminopyridin-3-yl)-5- phenyl-3H-imidazo[4,5-b]pyridin-3- yl)phenyl)ethyl)-3-formyl-4- hydroxybenzamide |
| 141 | 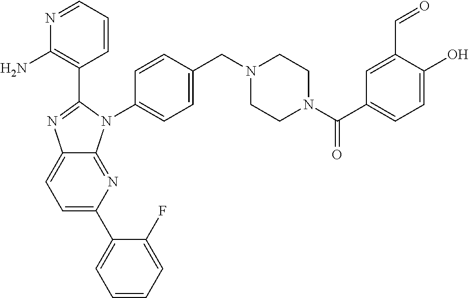 [SMILES: Nc1ncccc1-c1nc2ccc(-c3ccccc3F)nc2n1-c1ccc(CN2CCN(C(=O)c3ccc(O)c(C=O)c3)CC2)cc1] | 5-(4-(4-(2-(2-aminopyridin-3-yl)-5-(2- fluorophenyl)-3H-imidazo[4,5-b]pyridin-3- yl)benzyl)piperazine-1-carbonyl)-2- hydroxybenzaldehyde |
| 142 | 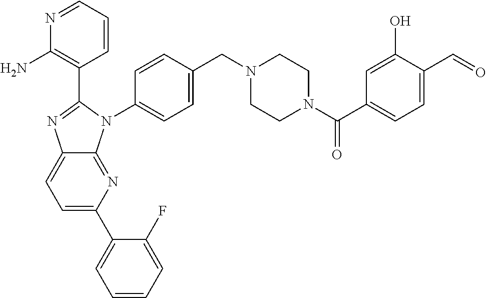 [SMILES: Nc1ncccc1-c1nc2ccc(-c3ccccc3F)nc2n1-c1ccc(CN2CCN(C(=O)c3ccc(C=O)c(O)c3)CC2)cc1] | 4-(4-(4-(2-(2-aminopyridin-3-yl)-5-(2- fluorophenyl)-3H-imidazo[4,5-b]pyridin-3- yl)benzyl)piperazine-1-carbonyl)-2- hydroxybenzaldehyde |
| 143 | 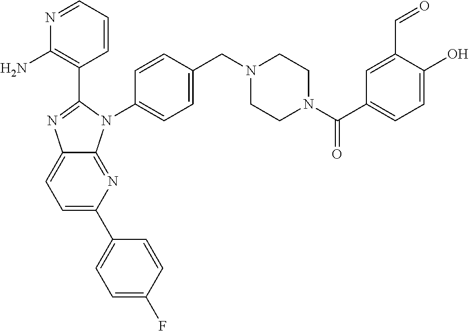 [SMILES: Nc1ncccc1-c1nc2ccc(-c3ccc(F)cc3)nc2n1-c1ccc(CN2CCN(C(=O)c3ccc(O)c(C=O)c3)CC2)cc1] | 5-(4-(4-(2-(2-aminopyridin-3-yl)-5-(4- fluorophenyl)-3H-imidazo[4,5-b]pyridin-3- yl)benzyl)piperazine-1-carbonyl)-2- hydroxybenzaldehyde |
| 144 | 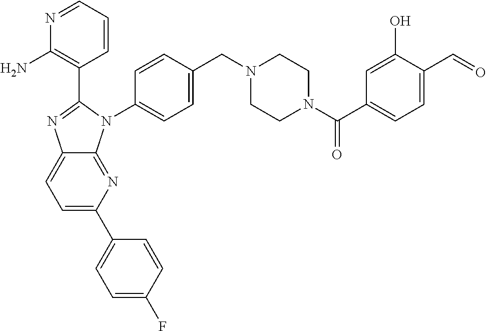 [SMILES: Nc1ncccc1-c1nc2ccc(-c3ccc(F)cc3)nc2n1-c1ccc(CN2CCN(C(=O)c3ccc(C=O)c(O)c3)CC2)cc1] | 4-(4-(4-(2-(2-aminopyridin-3-yl)-5-(4- fluorophenyl)-3H-imidazo[4,5-b]pyridin-3- yl)benzyl)piperazine-1-carbonyl)-2- hydroxybenzaldehyde |
| 145 | 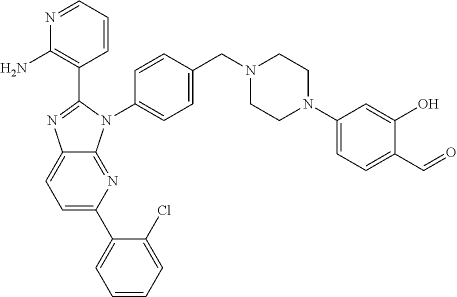 [SMILES: Nc1ncccc1-c1nc2ccc(-c3ccccc3Cl)nc2n1-c1ccc(CN2CCN(c3ccc(C=O)c(O)c3)CC2)cc1] | 4-(4-(4-(2-(2-aminopyridin-3-yl)-5-(2- chlorophenyl)-3H-imidazo[4,5-b]pyridin-3- yl)benzyl)piperazin-1-yl)-2- hydroxybenzaldehyde |
| 146 | 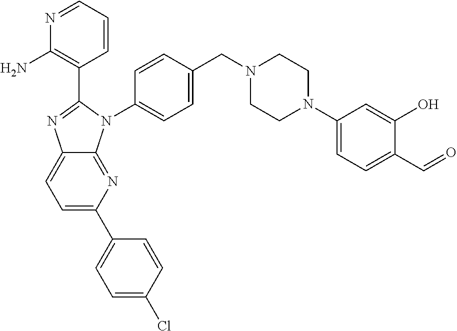 [SMILES: Nc1ncccc1-c1nc2ccc(-c3ccc(Cl)cc3)nc2n1-c1ccc(CN2CCN(c3ccc(C=O)c(O)c3)CC2)cc1] | 4-(4-(4-(2-(2-aminopyridin-3-yl)-5-(4- chlorophenyl)-3H-imidazo[4,5-b]pyridin-3- yl)benzyl)piperazin-1-yl)-2- hydroxybenzaldehyde |
| 147 | 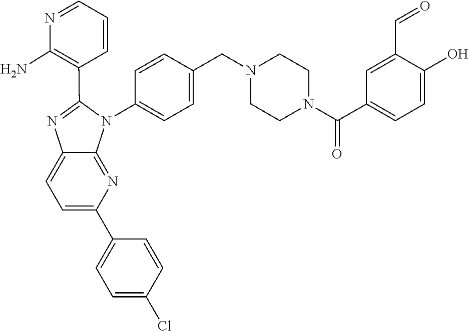 [SMILES: Nc1ncccc1-c1nc2ccc(-c3ccc(Cl)cc3)nc2n1-c1ccc(CN2CCN(C(=O)c3ccc(O)c(C=O)c3)CC2)cc1] | 5-(4-(4-(2-(2-aminopyridin-3-yl)-5-(4- chlorophenyl)-3H-imidazo[4,5-b]pyridin-3- yl)benzyl)piperazine-1-carbonyl)-2- hydroxybenzaldehyde |
| 148 | 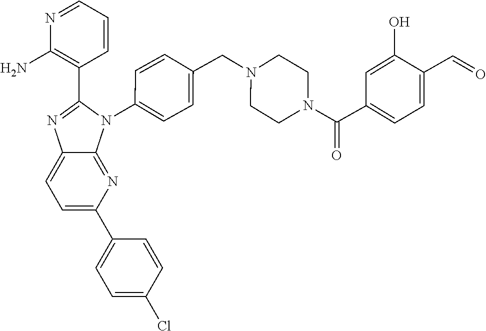 [SMILES: Nc1ncccc1-c1nc2ccc(-c3ccc(Cl)cc3)nc2n1-c1ccc(CN2CCN(C(=O)c3ccc(C=O)c(O)c3)CC2)cc1] | 4-(4-(4-(2-(2-aminopyridin-3-yl)-5-(4- chlorophenyl)-3H-imidazo[4,5-b]pyridin-3- yl)benzyl)piperazine-1-carbonyl)-2- hydroxybenzaldehyde |
| 149 | 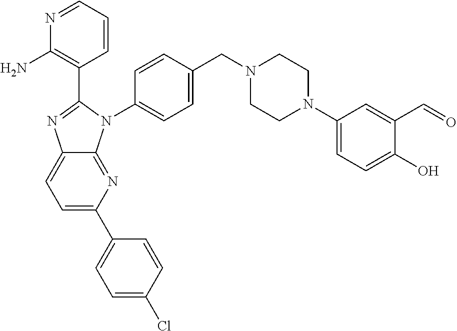 [SMILES: Nc1ncccc1-c1nc2ccc(-c3ccc(Cl)cc3)nc2n1-c1ccc(CN2CCN(c3ccc(O)c(C=O)c3)CC2)cc1] | 5-(4-(4-(2-(2-aminopyridin-3-yl)-5-(4- chlorophenyl)-3H-imidazo[4,5-b]pyridin-3- yl)benzyl)piperazin-1-yl)-2- hydroxybenzaldehyde |
| 150 | 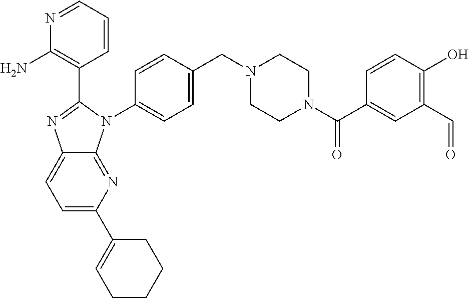 [SMILES: Nc1ncccc1-c1nc2ccc(C3=CCCCC3)nc2n1-c1ccc(CN2CCN(C(=O)c3ccc(O)c(C=O)c3)CC2)cc1] | 5-(4-(4-(2-(2-aminopyridin-3-yl)-5- (cyclohex-1-en-1-yl)-3H-imidazo[4,5- b]pyridin-3-yl)benzyl)piperazine-1- carbonyl)-2-hydroxybenzaldehyde |
| 151 | 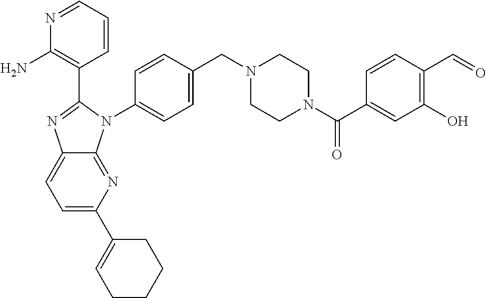 [SMILES: Nc1ncccc1-c1nc2ccc(C3=CCCCC3)nc2n1-c1ccc(CN2CCN(C(=O)c3ccc(C=O)c(O)c3)CC2)cc1] | 4-(4-(4-(2-(2-aminopyridin-3-yl)-5- (cyclohex-1-en-1-yl)-3H-imidazo[4,5- b]pyridin-3-yl)benzyl)piperazine-1- carbonyl)-2-hydroxybenzaldehyde |
| 152 | 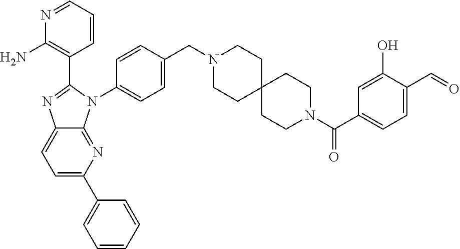 [SMILES: Nc1ncccc1-c1nc2ccc(-c3ccccc3)nc2n1-c1ccc(CN2CCC3(CC2)CCN(C(=O)c2ccc(C=O)c(O)c2)CC3)cc1] | 4-(9-(4-(2-(2-aminopyridin-3-yl)-5-phenyl- 3H-imidazo[4,5-b]pyridin-3-yl)benzyl)- 3,9-diazaspiro[5.5]undecane-3- carbonyl)-2-hydroxybenzaldehyde |
| 153 | 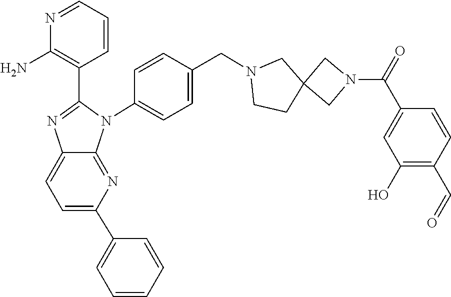 [SMILES: Nc1ncccc1-c1nc2ccc(-c3ccccc3)nc2n1-c1ccc(CN2CCC3(C2)CN(C(=O)c2ccc(C=O)c(O)c2)C3)cc1] | 4-(6-(4-(2-(2-aminopyridin-3-yl)-5-phenyl- 3H-imidazo[4,5-b]pyridin-3-yl)benzyl)- 2,6-diazaspiro[3.4]octane-2-carbonyl)-2- hydroxybenzaldehyde |

Another embodiment provides an AKT 1 inhibitory compound, or a pharmaceutically acceptable salt or solvate thereof, having a structure presented in Table 2.

TABLE 2

| | |
|---|---|
| 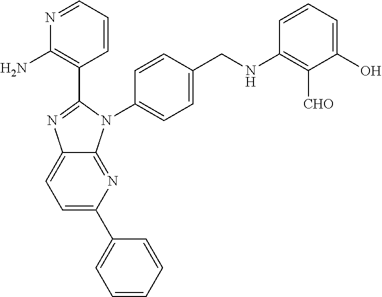 [SMILES: Nc1ncccc1-c1nc2ccc(-c3ccccc3)nc2n1-c1ccc(CNc2cccc(O)c2C=O)cc1] | 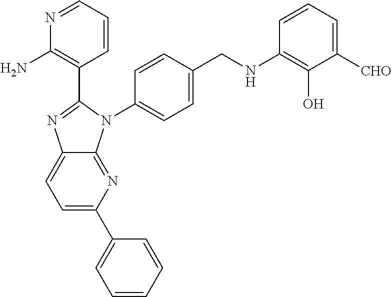 [SMILES: Nc1ncccc1-c1nc2ccc(-c3ccccc3)nc2n1-c1ccc(CNc2cccc(C=O)c2O)cc1] |
| 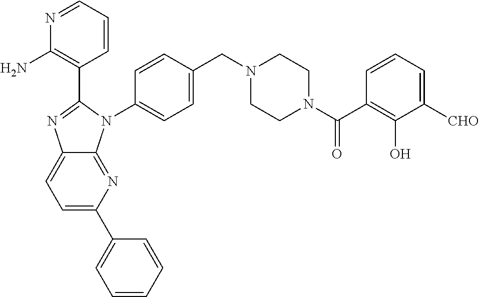 [SMILES: Nc1ncccc1-c1nc2ccc(-c3ccccc3)nc2n1-c1ccc(CN2CCN(C(=O)c3cccc(C=O)c3O)CC2)cc1] | 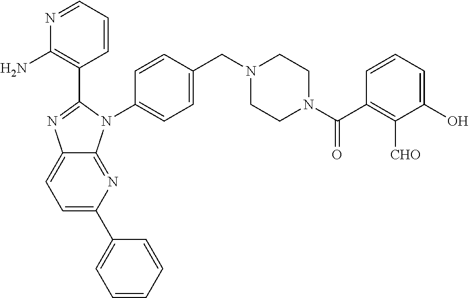 [SMILES: Nc1ncccc1-c1nc2ccc(-c3ccccc3)nc2n1-c1ccc(CN2CCN(C(=O)c3cccc(O)c3C=O)CC2)cc1] |
| 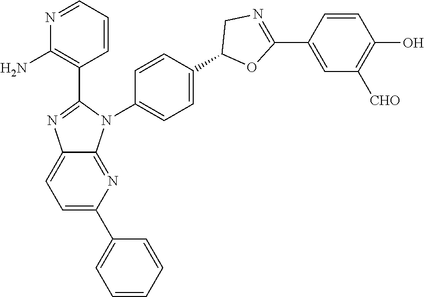 [SMILES: Nc1ncccc1-c1nc2ccc(-c3ccccc3)nc2n1-c1ccc([C@@H]2CN=C(c3ccc(O)c(C=O)c3)O2)cc1] | 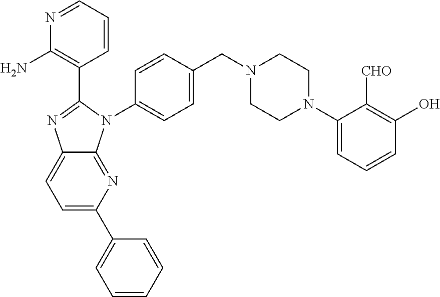 [SMILES: Nc1ncccc1-c1nc2ccc(-c3ccccc3)nc2n1-c1ccc(CN2CCN(c3cccc(O)c3C=O)CC2)cc1] |
| 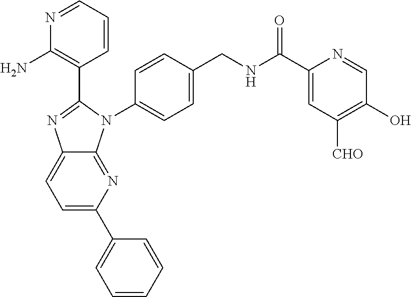 [SMILES: Nc1ncccc1-c1nc2ccc(-c3ccccc3)nc2n1-c1ccc(CNC(=O)c2cc(C=O)c(O)cn2)cc1] | 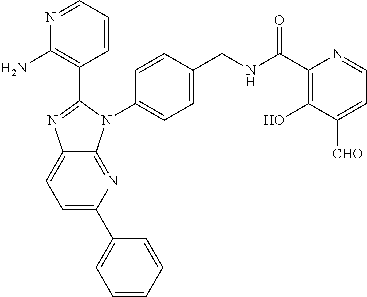 [SMILES: Nc1ncccc1-c1nc2ccc(-c3ccccc3)nc2n1-c1ccc(CNC(=O)c2nccc(C=O)c2O)cc1] |
| 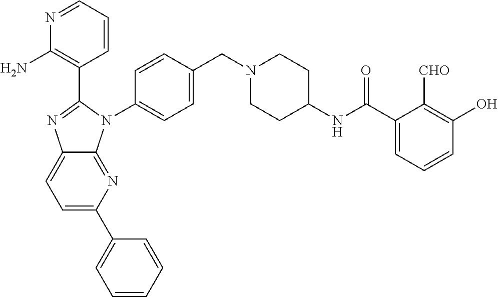 [SMILES: Nc1ncccc1-c1nc2ccc(-c3ccccc3)nc2n1-c1ccc(CN2CCC(NC(=O)c3cccc(O)c3C=O)CC2)cc1] | 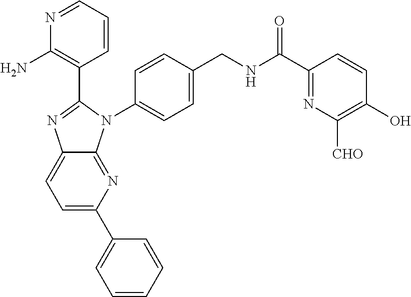 [SMILES: Nc1ncccc1-c1nc2ccc(-c3ccccc3)nc2n1-c1ccc(CNC(=O)c2ccc(O)c(C=O)n2)cc1] |
| 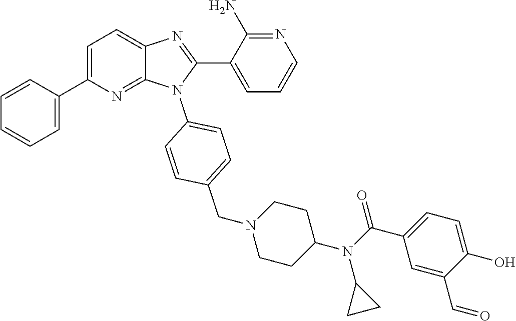 [SMILES: Nc1ncccc1-c1nc2ccc(-c3ccccc3)nc2n1-c1ccc(CN2CCC(N(C(=O)c3ccc(O)c(C=O)c3)C3CC3)CC2)cc1] | |

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa. CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the AKT1 inhibitory compound described herein is administered as a pure chemical. In other embodiments, the AKT1 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one AKT1 inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the AKT1 inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Formula (II), or a pharmaceutically acceptable salt or solvate thereof.

One embodiment provides a method of preparing a pharmaceutical composition comprising mixing a compound of Formula (II), or a pharmaceutically acceptable salt or solvate thereof, and a pharmaceutically acceptable carrier.

In certain embodiments, the AKT1 inhibitory compound as described by Formula (II), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt or solvate thereof.

One embodiment provides a method of preparing a pharmaceutical composition comprising mixing a compound of Table 1 or Table 2, or a pharmaceutically acceptable salt or solvate thereof, and a pharmaceutically acceptable carrier.

In certain embodiments, the AKT1 inhibitory compound as described by Table 1 or Table 2, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the AKT1 inhibitory compound as described by Formula (I) or Table 1 or Table 2, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one AKT1 inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a pharmaceutical composition comprising a compound of Formula (I), or a pharmaceutically acceptable salt or solvate thereof, and a pharmaceutically acceptable excipient for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), or a pharmaceutically acceptable salt or solvate thereof. In some embodiments is provided a method of treating cancer, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Formula (Ia), or a pharmaceutically acceptable salt or solvate thereof, for use in a method of treatment of the human or animal body.

One embodiment provides a compound of Formula (Ia), or a pharmaceutically acceptable salt or solvate thereof, for use in a method of treatment of cancer or neoplastic disease.

One embodiment provides a pharmaceutical composition comprising a compound of Formula (Ia), or a pharmaceutically acceptable salt or solvate thereof, and a pharmaceutically acceptable excipient for use in a method of treatment of cancer or neoplastic disease.

One embodiment provides a use of a compound of Formula (Ia), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (Ia), or a pharmaceutically acceptable salt or solvate thereof. In some embodiments is provided a method of treating cancer, in a patient in need thereof, comprising administering to the patient a pharmaceutical composition comprising a compound of Formula (Ia), or a pharmaceutically acceptable salt or solvate thereof, and a pharmaceutically acceptable excipient.

One embodiment provides a compound of Formula (II), or a pharmaceutically acceptable salt or solvate thereof, for use in a method of treatment of the human or animal body.

One embodiment provides a compound of Formula (II), or a pharmaceutically acceptable salt or solvate thereof, for use in a method of treatment of cancer or neoplastic disease.

One embodiment provides a pharmaceutical composition comprising a compound of Formula (II), or a pharmaceutically acceptable salt or solvate thereof, and a pharmaceutically acceptable excipient for use in a method of treatment of cancer or neoplastic disease.

One embodiment provides a use of a compound of Formula (II), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (II), or a pharmaceutically acceptable salt or solvate thereof. In some embodiments is provided a method of treating cancer, in a patient in need thereof, comprising administering to the patient a pharmaceutical composition comprising a compound of Formula (II), or a pharmaceutically acceptable salt or solvate thereof, and a pharmaceutically acceptable excipient.

One embodiment provides a compound of Table 1 or Table 2, or a pharmaceutically acceptable salt or solvate thereof, for use in a method of treatment of the human or animal body.

One embodiment provides a compound of Table 1 or Table 2, or a pharmaceutically acceptable salt or solvate thereof, for use in a method of treatment of cancer or neoplastic disease.

One embodiment provides a pharmaceutical composition comprising a compound of Table 1 or Table 2, or a pharmaceutically acceptable salt or solvate thereof, and a pharmaceutically acceptable excipient for use in a method of treatment of cancer or neoplastic disease.

One embodiment provides a use of a compound of Table 1 or Table 2, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1 or Table 2, or a pharmaceutically acceptable salt or solvate thereof. In some embodiments is provided a method of treating cancer, in a patient in need thereof, comprising administering to the patient a pharmaceutical composition comprising a compound of Table 1 or Table 2, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting a AKT1 enzyme comprising contacting the AKT1 enzyme with a compound of Formula (I), (II), or Table 1 or Table 2.

Another embodiment provides the method of inhibiting a AKT1 enzyme, wherein the AKT1 enzyme is contacted in an in vivo setting. Another embodiment provides the method of inhibiting a AKT1 enzyme, wherein the AKT1 enzyme is contacted in an in vitro setting.

Other embodiments and uses will be apparent to one skilled in the art in light of the present disclosures. The following examples are provided merely as illustrative of various embodiments and shall not be construed to limit the invention in any way.

### Examples

### 1. Chemical Synthesis

In some embodiments, the AKT1 inhibitory compounds disclosed herein are synthesized according to the following examples. As used below, and throughout the description of the invention, the following abbreviations, unless otherwise indicated, shall be understood to have the following meanings:
ACN acetonitrile
°C degrees Celsius
δ_{H} chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP Triphenylphosphine

### Experimental Procedures

### Intermediate 1: 3-(3-(4-(Aminomethyl)phenyl)-3H-imidazo[4,5-b]pyridin-2-yl)pyridin-2-amine

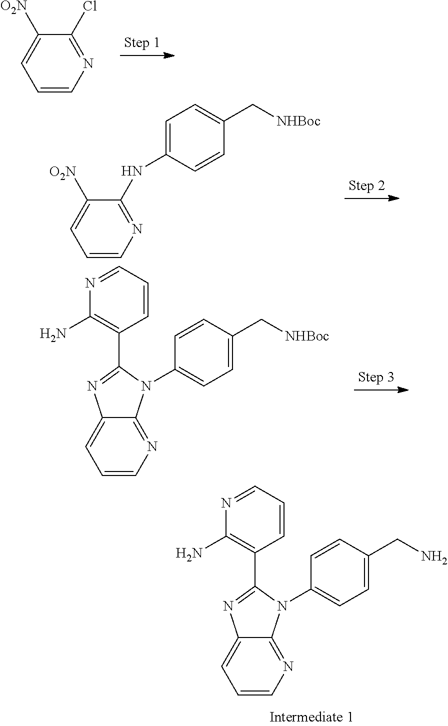

### Step 1: tert-Butyl 4-((3-nitropyridin-2-yl)amino)benzylcarbamate

To a solution of 2-chloro-3-nitro-pyridine (7.0 g, 44.2 mmol) and tert-butyl N-[(4-aminophenyl)methyl]carbamate (9.8 g, 44.2 mmol) in DMSO (100 mL) was added DIEA (11.4 g, 88.3 mmol). The mixture was stirred at 80 °C for 12 hr. The reaction mixture was diluted with H₂O (100 mL) at 25 °C and extracted with EtOAc (100 mL×3). The combined organic layers were washed with brine (150 mL), dried over Na₂SO₄, filtered, and concentrated under reduced pressure to give a residue. The residue was triturated (petroleum ether:EtOAc=10:1) to give tert-butyl N-[[4-[(3-nitro-2-pyridyl)amino]phenyl]methyl]carbamate (13.9 g, yield: 91%). MS: m/z=344.8 [M+H]⁺. ¹H NMR (400 MHz, Dimethylsulfoxide-d₆) δ 9.90 (s, 1H), 8.49 (dd, J=8.4, 1.6 Hz, 1H), 8.45 (dd, J=4.4, 1.6 Hz, 1H), 7.52 (d, J=8.4 Hz, 2H), 7.35 (t, J=6.0 Hz, 1H), 7.19 (d, J=8.4 Hz, 2H), 6.93 (dd, J=8.4, 4.4 Hz, 1H), 4.07 (d, J=6.0 Hz, 2H), 1.36 (s, 9H).

### Step 2: tert-Butyl 4-(2-(2-aminopyridin-3-yl)-3H-imidazo[4,5-b]pyridin-3-yl)benzylcarbamate

To a solution of tert-butyl N-[[4-[(3-nitro-2-pyridyl)amino]phenyl]methyl]carbamate (10 g, 29.0 mmol) in MeOH (70 mL) and DMSO (140 mL) were added 2-aminopyridine-3-carbaldehyde (3.9 g, 31.9 mmol) and Na₂S₂O₄ (10.1 g, 58.1 mmol). The mixture was stirred at 100 °C for 12 hr. After cooling to room temperature, the reaction mixture was diluted with H₂O (200 mL) and extracted with EtOAc (400 mL×2). The combined organic layers were washed with brine (400 mL), dried over Na₂SO₄, filtered, and concentrated under reduced pressure to give a residue. The crude product was purified by flash chromatography on silica gel (Eluent of 1-2% MeOH in CH₂Cl₂) to give tert-butyl 4-(2-(2-aminopyridin-3-yl)-3H-imidazo[4,5-b]pyridin-3-yl)benzylcarbamate (5.7 g, yield: 44%). MS: m/z=417.4 [M+H]⁺. ¹H NMR (400 MHz, Dimethylsulfoxide-d₆) δ 8.31 (dd, J=4.8, 1.2 Hz, 1H), 8.20 (dd, J=8.0, 1.2 Hz, 1H), 7.99 (dd, J=4.8, 1.6 Hz, 1H), 7.49 (t, J=6.0 Hz, 1H), 7.41-7.36 (m, 5H), 7.21 (dd, J=7.6, 1.6 Hz, 1H), 6.99 (br s, 2H), 6.39 (dd, J=7.6, 4.8 Hz, 1H), 4.21 (d, J=6.0 Hz, 2H), 1.41 (s, 9H).

### Step 3: 3-(3-(4-(Aminomethyl)phenyl)-3H-imidazo[4,5-b]pyridin-2-yl)pyridin-2-amine

To a solution of tert-butyl N-[[4-[2-(2-amino-3-pyridyl)imidazo[4,5-b]pyridin-3-yl]phenyl]methyl]carbamate (350 mg, 840 μmol) in 1,4-dioxane (3 mL) was added 4 M HCl in 1,4-dioxane (1 mL) at 20 °C The mixture was stirred at 20 °C for 2 hr. The reaction was concentrated under reduced pressure to give crude product 280 mg (HCl salt, yield: 95%). The crude product was purified by prep-HPLC (Column: Phenomenex luna C18 150×25 mm×10 μm; Condition: water (HCl)-ACN; Begin B: 0; End B: 16; Gradient Time (min): 10; 100% B Hold Time (min): 2; Flow Rate (mL/min): 25) to give product (HCl salt). The product was diluted with aqueous NaHCO₃ (10 mL) and extracted with DCM (10 mL×3). The combined organic layers were washed with brine (20 mL), dried over Na₂SO₄, filtered, and concentrated under reduced pressure to give 3-(3-(4-(Aminomethyl)phenyl)-3H-imidazo[4,5-b]pyridin-2-yl)pyridin-2-amine (Intermediate 1, 70.0 mg, yield: 95%). MS: m/z=317.1 [M+H]r. ¹H NMR (400 MHz, Dimethylsulfoxide-d₆) δ 8.31 (dd, J=4.8, 1.2 Hz, 1H), 8.19 (dd, J=8.0, 1.2 Hz, 1H), 7.99 (dd, J=4.8, 1.6 Hz, 1H), 7.47 (d, J=8.4 Hz, 2H), 7.40-7.33 (m, 3H), 7.22 (dd, J=7.6, 2.0 Hz, 1H), 6.98 (br s, 2H), 6.40 (dd, J=7.6, 4.8 Hz, 1H), 3.79 (s, 2H), 1.82 (br s, 2H).

### Intermediate 2: 3-(3-(4-(Aminomethyl)phenyl)-5-phenyl-3H-imidazo[4,5-b]pyridin-2-ylpyridin-2-amine

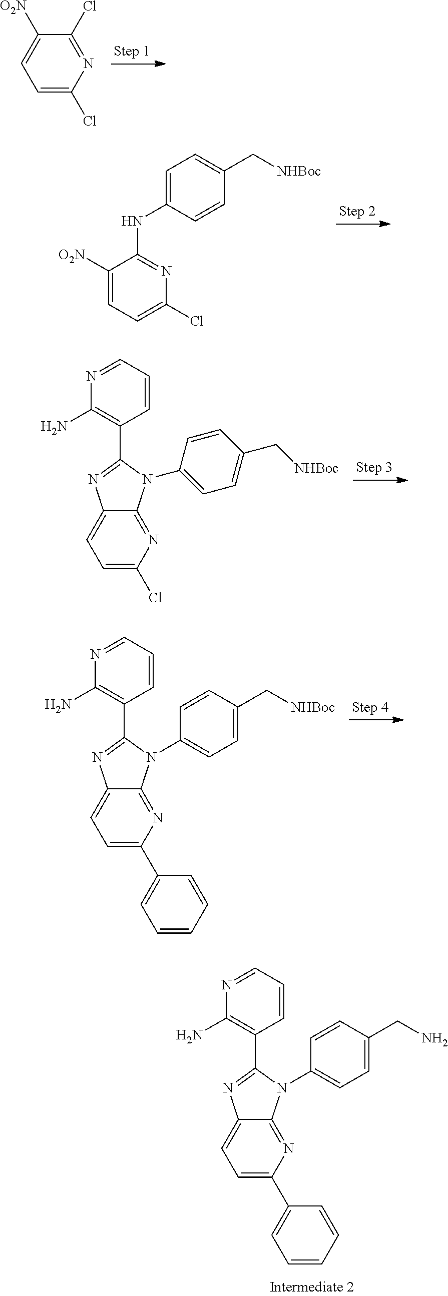

### Step 1: tert-Butyl 4-((6-chloro-3-nitropyridin-2-yl)amino)benzylcarbamate

To a solution of 2,6-dichloro-3-nitro-pyridine (2.0 g, 10.4 mmol) and tert-butyl N-[(4-aminophenyl)methyl]carbamate (2.3 g, 10.4 mmol) in DMSO (25 mL) was added DIEA (4.0 g, 31.1 mmol). The mixture was stirred at 80 °C for 12 hr. The reaction mixture was quenched with H₂O (50 mL) at 25 °C and extracted with EtOAc (50 mL×3). The combined organic layers were washed with brine (50 mL×2), dried over Na₂SO₄, filtered, and concentrated under reduced pressure to give crude product. The crude product was purified by flash chromatography on silica gel (Eluent of 0~10% MeOH in CH₂Cl₂) to give tert-butyl N-[[4-[(6-chloro-3-nitro-2-pyridyl)amino]phenyl]methyl]carbamate (2.4 g, yield: 44%). MS: m/z=400.9 [M+Na]⁺. ¹H NMR (400 MHz, Dimethylsulfoxide-di) δ 10.10 (s, 1H), 8.53 (d, J=8.8 Hz, 11H), 7.52 (d, J=8.4 Hz, 2H), 7.40 (t, J=6.0 Hz, 1H), 7.25 (d, J=8.4 Hz, 2H), 6.99 (d, J=8.8 Hz, 1H), 4.13 (d, J=6.0 Hz, 2H), 1.40 (s, 9H).

### Step 2: tert-Butyl N-[[4-[2-(2-amino-3-pyridyl)-5-chloro-imidazo[4,5-b]pyridin-3-yl]phenyl]methyl]carbamate

To a solution of tert-butyl N-[[4-[(6-chloro-3-nitro-2-pyridyl)amino]phenyl]methyl]carbamate (2.0 g, 5.3 mmol) in DMSO (30 mL) and MeOH (15 mL) were added 2-aminopyridine-3-carbaldehyde (0.7 g, 5.8 mmol) and Na₂S₂O₄ (1.8 g, 10.6 mmol). The mixture was stirred at 100 °C for 16 hr. The reaction mixture was quenched with H₂O (50 mL) at 25 °C and extracted with EtOAc (100 mL×3). The combined organic layers were washed with brine (100 mL×2), dried over Na₂SO₄, filtered, and concentrated under reduced pressure to give a crude product. The crude product was purified by flash chromatography on silica gel (Eluent of 0~10% MeOH in CH₂Cl₂) to give tert-butyl N-[[4-[2-(2-amino-3-pyridyl)-5-chloro-imidazo[4,5-b]pyridin-3-yl]phenyl]methyl]carbamate (1.4 g, yield: 51%). MS: m/z=451.0 [M+H]⁺. ¹H NMR (400 MHz, Dimethylsulfoxide-d₆) δ 8.26 (d, J=8.4 Hz, 1H), 7.99 (dd, J=4.8, 1.6 Hz, 1H), 7.50-7.43 (m, 2H), 7.38 (s, 4H), 7.21 (dd, J=7.6, 1.6 Hz, 1H), 6.91 (br s, 2H), 6.40 (dd, J=7.6, 4.8 Hz, 1H), 4.22 (d, J=6.0 Hz, 2H), 1.41 (s, 9H).

### Step 3: tert-Butyl N-[[4-[2-(2-amino-3-pyridyl)-5-phenyl-imidazo[4,5-b]pyridin-3-yl]phenyl]methyl]carbamate

To a solution of tert-butyl N-[[4-[2-(2-amino-3-pyridyl)-5-chloro-imidazo[4,5-b]pyridin-3-yl]phenyl]methyl]carbamate (500 mg, 1.1 mmol) and phenylboronic acid (270 mg, 2.2 mmol,) in 1,4-dioxane (5 mL) and H₂O (1 mL) were added Pd(dppf)Cl₂ (81.1 mg, 111 μmol) and Cs₂CO₃ (1.1 g, 3.3 mmol). The mixture was degassed, purged with N₂ three times, and stirred at 80 °C for 16 hr under N₂ atmosphere. After cooling to 20 °C, the reaction was diluted with EtOAc, (10 mL). The mixture was filtered through celite and extracted with H₂O (10 mL×3). The combined organic layers were washed with brine (15 mL×3), dried over Na₂SO₄, filtered and concentrated under reduced pressure to give a residue. The crude product was purified by flash chromatography on silica gel (Eluent of 10-35% EtOAc in petroleum ether) to give tert-butyl N-[[4-[2-(2-amino-3-pyridyl)-5-phenyl-imidazo[4,5-b]pyridin-3-yl]phenyl]methyl]carbamate (126 mg, yield: 21%). MS: m/z=493.2 [M+H]⁺.

### Step 4: 3-(3-(4-(Aminomethyl)phenyl)-5-phenyl-3H-imidazo[4,5-b]pyridin-2-yl)pyridin-2-amine

To a solution of tert-butyl N-[[4-[2-(2-amino-3-pyridyl)-5-phenyl-imidazo[4,5-b]pyridin-3-yl]phenyl]methyl]carbamate (126 mg, 256 μmol) in HCl/1,4-dioxane (4 M, 1 mL). The mixture was stirred at 25 °C for 2 hr. The solvent was removed under reduced pressure to give a crude product (84 mg, yield: 84%). The crude product was purified by prep-HPLC (column: Welch Xtimate C18 150×25 mm×5 μm; mobile phase: [water (HCl)-ACN]; B %: 5%-35%, 8 min) to give the desired product (HCl salt). The product was diluted with aqueous NaHCO₃ (10 mL) and extracted with DCM (10 mL×3). The combined organic layers were washed with brine (10 mL), dried over Na₂SO₄, filtered, and concentrated under reduced pressure to give 3-(3-(4-(aminomethyl)phenyl)-5-phenyl-3H-imidazo[4,5-b]pyridin-2-yl)pyridin-2-amine (Intermediate 2, 32.2 mg, yield: 84%). MS: m/z=393.1 [M+H]⁺. ¹H NMR (400 MHz, Dimethylsulfoxide-d₆) δ 8.26 (d, J=8.4 Hz, 1H), 8.04-7.96 (m, 4H), 7.54-7.49 (m, 2H), 7.49-7.43 (m, 2H), 7.43-7.35 (m, 3H), 7.21 (dd, J=7.6, 1.6 Hz, 1H), 6.98 (br s, 2H), 6.41 (dd, J=7.6, 4.8 Hz, 1H), 3.82 (s, 2H).

### Intermediate 3: 3-[5-Phenyl-3-[4-(piperazin-1-ylmethyl)phenyl]imidazo[4,5-b]pyridin-2-yl]pyridin-2-amine

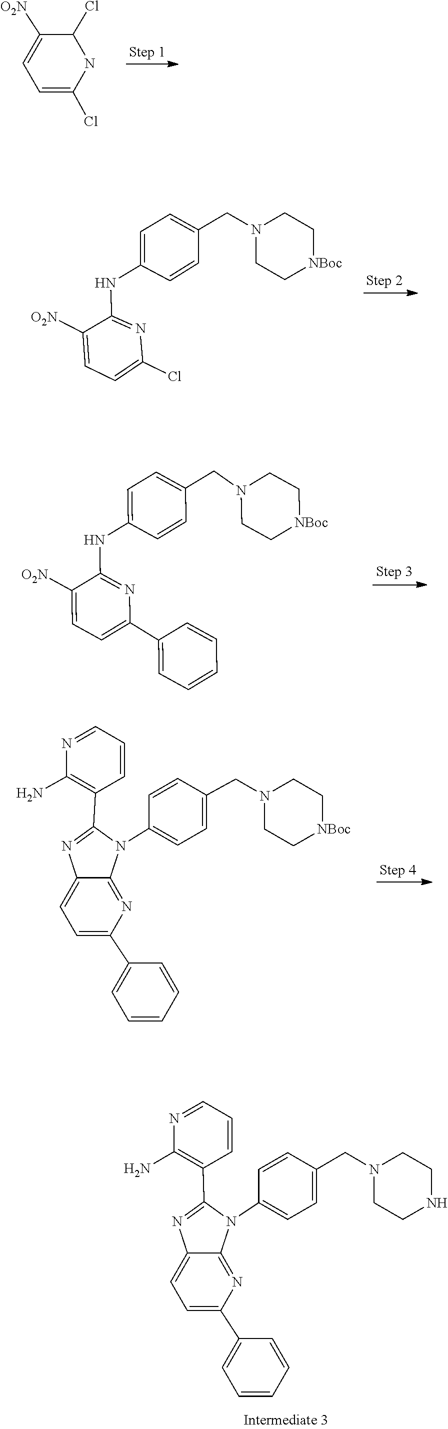

### Step 1: tert-Butyl 4-[[4-[(6-chloro-3-nitro-2-pyridyl)amino]phenyl]methyl]piperazine-1-carboxylate

To a solution of 2,6-dichloro-3-nitro-pyridine (5.0 g, 25.9 mmol) in 1,4-dioxane (50 mL) were added DIEA (6.7 g, 51.8 mmol) and tert-butyl 4-[(4-aminophenyl)methyl]piperazine-1-carboxylate (10.8 g, 25.9 mmol). The mixture was stirred at 60 °C for 12 hr. The reaction mixture was diluted H₂O (50 mL) and extracted with EtOAc (100 mL×3). The combined organic layers were washed with brine (50 mL×2), dried over Na₂SO₄, filtered, and concentrated under reduced pressure to give a residue. The residue was purified by flash silica gel chromatography (Eluent of 0~50% EtOAc in petroleum ether) to give tert-butyl 4-[[4-[(6-chloro-3-nitro-2-pyridyl)amino]phenyl]methyl]piperazine-1-carboxylate (6.1 g, yield: 53%). MS: m/z=447.9 [M+H]⁺. ¹H NMR (400 MHz, Chloroform-d) δ 10.27 (s, 1H), 8.46 (d, J=8.8 Hz, 1H), 7.61 (d, J=8.4 Hz, 2H), 7.36 (d, J=8.4 Hz, 2H), 6.80 (d, J=8.4 Hz, 1H), 3.52 (s, 2H), 3.48-3.37 (m, 4H), 2.45-2.32 (m, 4H), 1.46 (s, 9H).

### Step 2: tert-Butyl 4-[[4-[(3-nitro-6-phenyl-2-pyridyl)amino]phenyl]methyl]piperazine-1-carboxylate

To a solution of tert-butyl 4-[[4-[(6-chloro-3-nitro-2-pyridyl)amino]phenyl]methyl]piperazine-1-carboxylate (1.0 g, 2.23 mmol) and phenylboronic acid (544 mg, 4.47 mmol) in 1,4-dioxane (10 mL) and H₂O (2 mL) were added Pd(dppf)Cl₂ (327 mg, 0.446 mmol) and K₂CO₃ (926 mg, 6.7 mmol). The mixture was stirred at 60 °C for 4 hr. The reaction mixture was diluted with H₂O (50 mL) and extracted with EtOAc (100 mL×3). The combined organic layers were washed with brine (50 mL×2), dried over Na₂SO₄, filtered, and concentrated under reduced pressure to give a residue. The residue was purified by flash silica gel chromatography (Eluent of 0~50% EtOAc in petroleum ether) to give tert-butyl 4-[[4-[(3-nitro-6-phenyl-2-pyridyl)amino]phenyl]methyl]piperazine-1-carboxylate (1.0 g, yield: 92%). MS: m/z=490.1 [M+H]⁺. ¹H NMR (400 MHz, Chloroform-d) δ 10.31 (s, 1H), 8.59 (d, J=8.8 Hz, 1H), 8.06-8.04 (m, 2H), 7.73 (d, J=8.4 Hz, 2H), 7.52-7.44 (m, 3H), 7.37 (d, J=8.4 Hz, 2H), 7.3 (d, J=8.4 Hz, 1H), 3.54 (s, 2H), 3.52-3.37 (m, 4H), 2.49-2.37 (m, 4H), 1.46 (s, 9H).

### Step 3: tert-Butyl 4-[[4-[2-(2-amino-3-pyridyl)-5-phenyl-imidazo[4,5-b]pyridin-3-yl]phenyl]methyl]piperazine-1-carboxylate

To a solution of 2-aminopyridine-3-carbaldehyde (269 mg, 2.21 mmol) and tert-butyl 4-[[4-[(3-nitro-6-phenyl-2-pyridyl)amino]phenyl]methyl]piperazine-1-carboxylate (900 mg, 1.84 mmol) in DMSO (10 mL) was added Na₂S₂O₄ (960 mg, 5.52 mmol) at 15 °C The mixture was stirred at 100 °C for 20 hr. The reaction mixture was diluted with H₂O (50 mL) and extracted with DCM (80 mL×3). The combined organic layers were washed with brine (80 mL×3), dried over Na₂SO₄, filtered, and concentrated under reduced pressure to give a residue. The residue was purified by flash silica gel chromatography (Eluent of 0~10% MeOH in CH₂Cl₂) to give tert-butyl 4-[[4-[2-(2-amino-3-pyridyl)-5-phenyl-imidazo[4,5-b]pyridin-3-yl]phenyl]methyl]piperazine-1-carboxylate (600 mg, yield: 58%). MS: m/z=562.1 [M+H]⁺. ¹H NMR (400 MHz, Chloroform-d) δ 8.13 (d, J=8.4 Hz, 1H), 8.06 (dd, J=5.2, 2.0 Hz, 1H), 8.03-8.01 (m, 2H), 7.81 (d, J=8.4 Hz, 1H), 7.51-7.49 (m, 2H), 7.48-7.35 (m, 5H), 7.10 (dd, J=9.6, 2.0 Hz, 1H), 6.66 (br s, 2H), 6.36 (dd, J=8.0, 4.0 Hz, 1H), 3.64 (s, 2H), 3.53-3.42 (m, 4H), 2.55-2.42 (m, 4H), 1.47 (s, 9H).

### Step 4: 3-[5-Phenyl-3-[4-(piperazin-1-ylmethyl)phenyl]imidazo[4,5-b]pyridin-2-yl]pyridin-2-amine

A solution of tert-butyl 4-[[4-[2-(2-amino-3-pyridyl)-5-phenyl-imidazo[4,5-b]pyridin-3-yl]phenyl]methyl]piperazine-1-carboxylate (400 mg, 712 μmol) in HCl/1,4-dioxane (4M, 8 mL) was stirred at 25 °C for 4 hr. The reaction was filtered and concentrated under reduced pressure to give 3-[5-phenyl-3-[4-(piperazin-1-ylmethyl)phenyl]imidazo[4,5-b]pyridin-2-yl]pyridin-2-amine (450 mg, HCl salt) as a yellow solid, which was used in the next step without further purification. The residue (200 mg) was purified by prep-HPLC (column: Waters xbridge 150*25 mm 10 um; mobile phase: [water(NH₄HCO₃)-ACN]; B %: 19%-49%, 9 min) to give 3-[5-Phenyl-3-[4-(piperazin-1-ylmethyl)phenyl]imidazo[4,5-b]pyridin-2-yl]pyridin-2-amine (Intermediate 3, 49.7 mg). MS: m/z=462.2 [M+H]⁺. ¹H NMR (400 MHz, Dimethylsulfoxide-d₆) δ 8.27 (d, J=8.4 Hz, 1H), 8.04-7.97 (m, 4H), 7.48-7.39 (m, 8H), 7.15 (dd, J=8.0, 2.0 Hz, 1H), 7.03 (br s, 2H), 6.38 (dd, J=7.6, 4.8 Hz, 1H), 3.53 (s, 2H), 2.74-2.66 (m, 4H), 2.38-2.27 (m, 4H).

### Intermediate 4: 3-[3-[4-[(4-amino-1-piperidyl)methyl]phenyl]-5-phenyl-imidazo[4,5-b]pyridin-2-yl]pyridin-2-amine

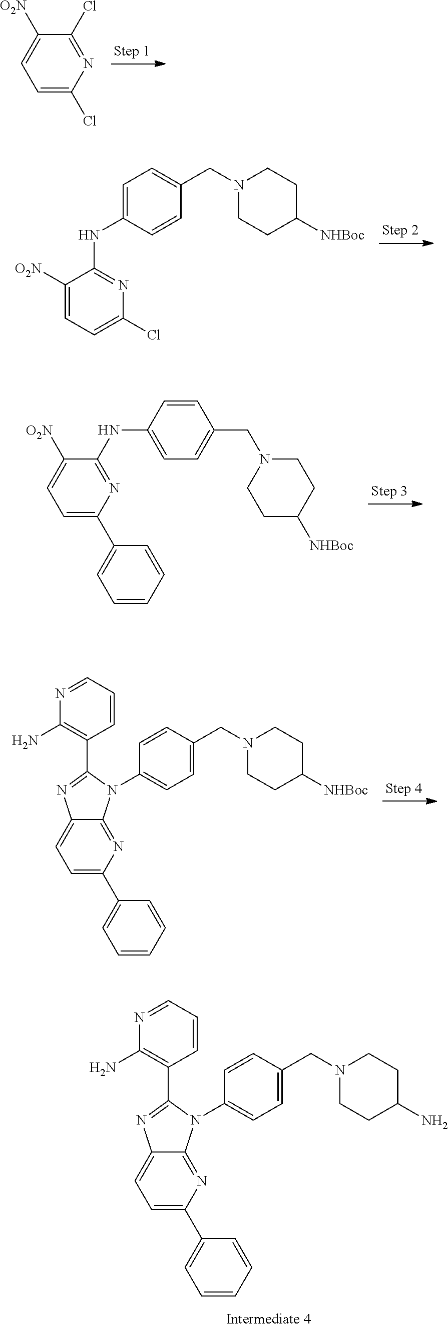 [SMILES: O=[N+]([O-])c1ccc(Cl)nc1Cl ; CC(C)(C)OC(=O)NC1CCN(Cc2ccc(Nc3nc(Cl)ccc3[N+](=O)[O-])cc2)CC1 ; CC(C)(C)OC(=O)NC1CCN(Cc2ccc(Nc3nc(-c4ccccc4)ccc3[N+](=O)[O-])cc2)CC1 ; CC(C)(C)OC(=O)NC1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCC(N)CC2)cc1]

### Step 1: tert-Butyl (1-(4-((6-chloro-3-nitropyridin-2-yl)amino)benzyl)piperidin-4-yl)carbamate

To a solution of 2,6-dichloro-3-nitro-pyridine (3.0 g, 15.5 mmol) and tert-butyl N-[1-[(4-aminophenyl)methyl]-4-piperidyl]carbamate (4.8 g, 15.6 mmol) in 1,4-dioxane (100 mL) was added DIEA (6.0 g, 46.6 mmol). The mixture was stirred at 50 °C for 12 hr. The reaction mixture was diluted with H₂O (50 mL) and extracted with EtOAc (30 mL×2). The combined organic layers were washed with brine (50 mL), dried over Na₂SO₄, filtered, and concentrated under reduced pressure to give a residue. The residue was purified by flash silica gel chromatography (Eluent of 0~5% MeOH in CH₂Cl₂) to give tert-butyl N-[1-[[4-[(6-chloro-3-nitro-2-pyridyl)amino]phenyl]methyl]-4-piperidyl]carbamate (4.6 g, yield: 64%). MS: m/z=462.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.42 (br s, 1H), 3.48 (s, 2H), 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 2: tert-butyl (1-(4-((3-nitro-6-phenylpyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.20 mmol), phenylboronic acid (528 mg, 4.30 mmol), Pd(dppf)Cl₂ (158 mg, 0.216 mmol) and K₂CO₃ (898 mg, 6.50 mmol) in 1,4-dioxane (10 mL) and H₂O (2 mL) was degassed and purged with N₂ three times. The mixture was stirred at 100 °C for 12 hr under N₂ atmosphere. After cooling to 25 °C, the reaction mixture was diluted with H₂O and extracted with DCM (50 mL×2). The combined organic layers were washed with brine (50 mL), dried over Na₂SO₄, filtered, and concentrated under reduced pressure to give a residue. The residue was purified by flash silica gel chromatography (Eluent of 0~7% MeOH in CH₂Cl₂) to give tert-butyl N—[I-[[4-[(3-nitro-6-phenyl-2-pyridyl)amino]phenyl]methyl]-4-piperidyl]carbamate (1.1 g, yield: 96%). MS: m/z=504.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.43 (br s, 1H), 3.48 (s, 2H), 3.41-3.46 (m, 1H). 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 3: tert-Butyl (1-(4-(2-(2-Aminopyridin-3-yl)-5-phenyl-3H-imidazo[4,5-b]pyridin-3-yl)benzyl)piperidin-4-yl) carbamate

A solution of tert-butyl N-[1-[[4-[(3-nitro-6-phenyl-2-pyridyl)amino]phenyl]methyl]-4-piperidyl]carbamate (200 mg, 0.397 mmol), 2-aminopyridine-3-carbaldehyde (53.4 mg, 0.437 mmol) and Na₂S₂O₄ (207 mg, 1.2 mmol) in DMSO (6 mL) was stirred at 100 °C for 18 hr. After cooling to 25 °C, the reaction mixture was diluted with DCM (40 mL). The organic layers were washed with H₂O (20 mL) and brine (20 mL), dried over Na₂SO₄, filtered and concentrated under reduced pressure to give a residue. The residue was purified by flash silica gel chromatography (IEluent of 0~7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]carbamate (100 mg, yield: 40%). MS: m/z=576.2 [M+H]⁺.

### Step 4: 3-(3-(4-((4-Aminopiperidin-1-yl)methyl)phenyl)-5-phenyl-3H-imidazo[4,5-b]pyridin-2-yl)pyridin-2-amine

A solution of tert-butyl (1-(4-(2-(2-aminopyridin-3-yl)-5-phenyl-3H-imidazo[4,5-b]pyridin-3-yl)benzyl)piperidin-4-yl)carbamate (200 mg, 0.347 mmol) in HCl in 1,4-dioxane (4 M, 2 mL) was stirred at 25 °C for 2 hr. The reaction mixture was concentrated under reduced pressure to give a residue. The residue was purified by prep-HPLC (column: Waters xbridge 150*25 mm 10 um; mobile phase: [water (NH₄HCO₃)-ACN]; B %: 24%-54%, 8 min) to give 3-[3-[4-[(4-amino-1-piperidyl)methyl]phenyl]-5-phenyl-imidazo[4,5-b]pyridin-2-yl]pyridin-2-amine (Intermediate 4, 120 mg, yield: 72%). MS: m/z=476.2 [M+H]⁺. ¹H NMR (400 MHz, Chloroform-d) δ 8.12 (d, J=8.4 Hz, 1H), 8.05 (dd, J=5.2, 1.6 Hz, 1H), 8.01 (d, J=7.2 Hz, 2H), 7.79 (d, J=8.4 Hz, 1H), 7.50-7.35 (m, 7H), 7.09 (dd, J=7.6, 1.2 Hz, 1H), 6.61 (br s, 2H), 6.35 (dd, J=7.6, 4.8 Hz, 1H), 3.58 (s, 2H), 2.88 (br d, J=11.6 Hz, 2H), 2.75-2.65 (m, 1H), 2.09 (br t, J=11.6 Hz, 2H), 1.83 (br d, J=11.6 Hz, 2H), 1.49-1.38 (m, 2H).

### Intermediate 5: 3-(3-(4-(Chloromethyl)phenyl)-5-phenyl-3H-imidazo[4,5-b]pyridin-2-yl)pyridin-2-amine

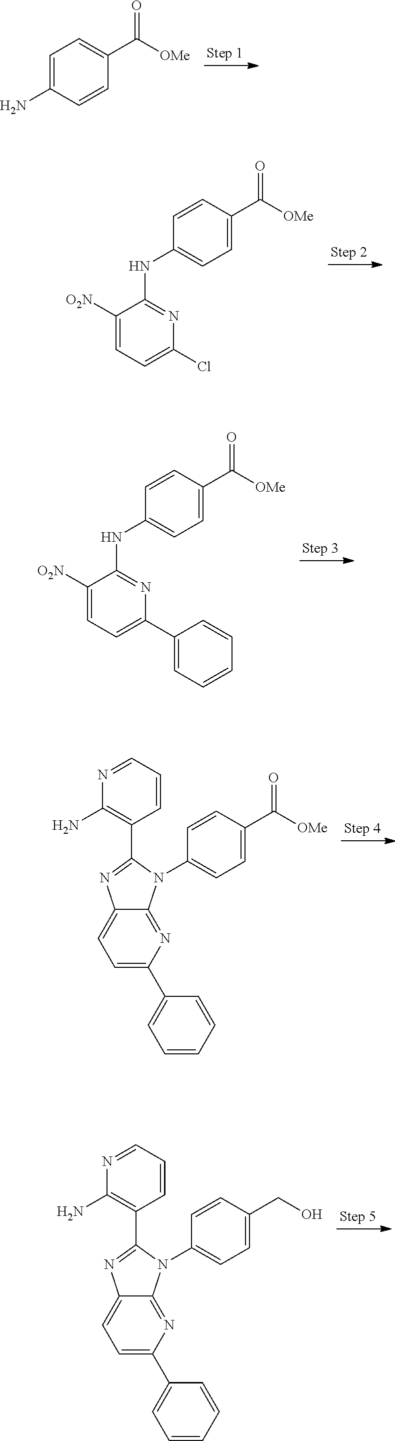 [SMILES: COC(=O)c1ccc(N)cc1 ; COC(=O)c1ccc(Nc2nc(Cl)ccc2[N+](=O)[O-])cc1 ; COC(=O)c1ccc(Nc2nc(-c3ccccc3)ccc2[N+](=O)[O-])cc1 ; COC(=O)c1ccc(-n2c(-c3cccnc3N)nc3ccc(-c4ccccc4)nc32)cc1 ; Nc1ncccc1-c1nc2ccc(-c3ccccc3)nc2n1-c1ccc(CO)cc1]

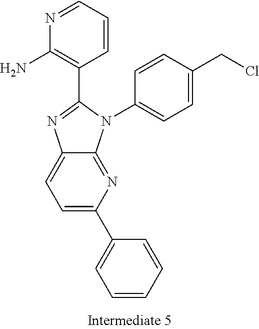 [SMILES: Nc1ncccc1-c1nc2ccc(-c3ccccc3)nc2n1-c1ccc(CCl)cc1]

### Step 1: Methyl 4-((6-chloro-3-nitropyridin-2-yl)amino)benzoate

To a solution of methyl 4-aminobenzoate (5 g, 33.1 mmol) in DMSO (50 mL) were added 2,6-dichloro-3-nitro-pyridine (7.66 g, 39.7 mmol) and DIEA (12.82 g, 99.2 mmol). The mixture was stirred at 80 °C for 16 hr. After cooling to 20 °C, the reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated to dryness. The crude product was triturated with EtOAc at 25 °C for 30 min to give methyl 4-[(6-chloro-3-nitro-2-pyridyl)amino]benzoate (8 g, yield: 51%). MS: m/z=307.8 [M+H]⁺. ¹H NMR (400 MHz, Dimethylsulfoxide-d₆) δ 10.25 (s, 1H), 8.57 (d, J=8.4 Hz, 1H), 7.97 (d, J=8.8 Hz, 2H), 7.79 (d, J=8.8 Hz, 2H), 7.12 (d, J=8.4 Hz, 1H), 3.85 (s, 3H).

### Step 2: Methyl 4-((3-nitro-6-phenylpyridin-2-yl)amino)benzoate

To a solution of methyl 4-[(6-chloro-3-nitro-2-pyridyl)amino]benzoate (45 g, 146 mmol) and phenylboronic acid (21.4 g, 176 mmol) in 1,4-dioxane (500 mL) and H₂O (100 mL) were added Pd(dppf)Cl₂ (10.7 g, 14.6 mmol) and Cs₂CO₃ (143 g, 439 mmol). The mixture was degassed, purged with N₂ three times, and stirred at 80 °C for 16 hr under N₂ atmosphere. The reaction mixture was poured into H₂O (500 mL) and extracted with CH₂Cl₂ (500 mL×3). The combined organic layers were washed with brine (500 mL×3), dried over anhydrous Na₂SO₄, filtered, and concentrated to dryness. The crude product was triturated with EtOAc at 25 °C for 30 min to give methyl 4-[(3-nitro-6-phenyl-2-pyridyl)amino]benzoate (35.2 g, yield: 69%). MS: m/z=350.0 [M+H]⁺. ¹H NMR (400 MHz, Dimethylsulfoxide-d₆) δ 10.25 (s, 1H), 8.62 (d, J=8.8 Hz, 1H), 8.15-8.10 (m, 2H), 8.00 (d, J=8.4 Hz, 2H), 7.92 (d, J=8.4 Hz, 2H), 7.67 (d, J=8.8 Hz, 1H), 7.59-7.54 (m, 3H), 3.86 (s, 3H).

### Step 3: Methyl 4-(2-(2-aminopyridin-3-yl)-5-phenyl-3H-imidazo[4,5-b]pyridin-3-yl)benzoate

To a solution of methyl 4-[(3-nitro-6-phenyl-2-pyridyl)amino]benzoate (15 g, 42.9 mmol) in DMSO (150 mL) were added 2-aminopyridine-3-carbaldehyde (6.29 g, 51.5 mmol) and Na₂S₂O₄ (15 g, 85.9 mmol). The reaction mixture was heated to 100 °C for 16 hr. After cooling to 25 °C, the reaction mixture was diluted with H₂O (200 mL) and extracted with CH₂Cl₂ (200 ml×3). The combined organic layers were washed with brine (200 ml×2), dried over Na₂SO₄, filtered, and concentrated under reduced pressure to give a residue. The crude product was triturated with CH₂Cl₂ at 25 °C for 30 min to give methyl 4-[2-(2-amino-3-pyridyl)-5-phenyl-imidazo[4,5-b]pyridin-3-yl]benzoate (12 g, yield: 66%). MS: m/z=422.0 [M+H]^{t}. ¹H NMR (400 MHz, Dimethylsulfoxide-d₆) δ 8.29 (d, J=8.0 Hz, 1H), 8.12 (d, J=8.4 Hz, 2H), 8.07-8.00 (m, 4H), 7.67 (d, J=8.8 Hz, 2H), 7.49-7.44 (m, 2H), 7.42-7.38 (m, 1H), 7.23 (dd, J=7.6, 1.6 Hz, 1H), 6.89 (br s, 2H), 6.46 (dd, J=7.6, 4.8 Hz, 1H), 3.90 (s, 3H).

### Step 4: (4-(2-(2-Aminopyridin-3-yl)-5-phenyl-3H-imidazo[4,5-b]pyridin-3-yl)phenyl)methanol

To a solution of methyl 4-[2-(2-amino-3-pyridyl)-5-phenyl-imidazo[4,5-b]pyridin-3-yl]benzoate (2.5 g, 5.9 mmol) in THF (25 mL) was added LiAlH₄ (450 mg, 11.9 mmol) at 0 °C After addition, the resulting mixture was stirred at 25 °C for 2 hr. After the reaction mixture was cooled to 0 °C, the reaction mixture was quenched with H₂O (100 mL), followed by 15% aqueous NaOH (30 mL). Then the reaction mixture was filtered. The filter liquor was concentrated to dryness to give [4-[2-(2-amino-3-pyridyl)-5-phenyl-imidazo[4,5-b]pyridin-3-yl]phenyl]methanol (1.87 g, yield: 80%), which was directly used to the next step without further purification. MS: m/z=394.1 [M+H]⁺.

### Step 5: 3-(3-(4-(Chloromethyl)phenyl)-5-phenyl-3H-imidazo[4,5-b]pyridin-2-yl)pyridin-2-amine

To a solution of [4-[2-(2-amino-3-pyridyl)-5-phenyl-imidazo[4,5-b]pyridin-3-yl]phenyl]methanol (2.3 g, 5.9 mmol) in CH₂Cl₂ (25 mL) was added SOCl₂ (2.1 g, 17.5 mmol). The mixture was stirred at 40 °C for 1 hr. The reaction mixture was filtered. The filter liquor was concentrated to dryness to give 3-[3-[4-(chloromethyl)phenyl]-5-phenyl-imidazo[4,5-b]pyridin-2-yl]pyridin-2-amine (Intermediate 5, 1.71 g, yield: 71%). MS: m/z=412.0 [M+H]⁺. ¹H NMR (400 MHz, Dimethylsulfoxide-d₆) δ 8.29 (d, J=8.4 Hz, 1H), 8.12 (d, J=8.4 Hz, 2H), 8.07-8.00 (m, 4H), 7.67 (d, J=8.4 Hz, 2H), 7.50-7.44 (m, 2H), 7.42-7.37 (m, 1H), 7.24 (d, J=7.2 Hz, 1H), 6.88 (br s, 2H), 6.46 (dd, J=4.8, 7.6 Hz, 1H), 3.90 (s, 2H).

### Intermediate 6: 2-hydroxy-4-piperazin-1-yl-benzaldehyde

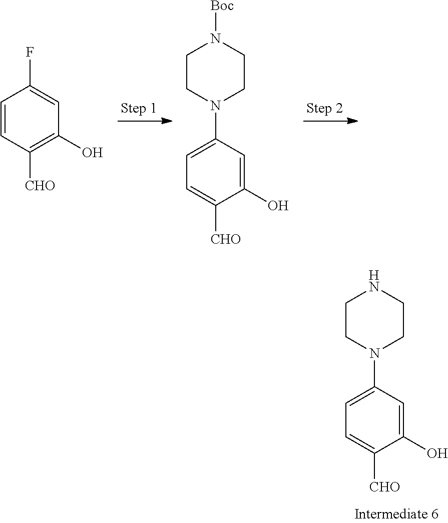 [SMILES: O=Cc1ccc(F)cc1O ; CC(C)(C)OC(=O)N1CCN(c2ccc(C=O)c(O)c2)CC1 ; O=Cc1ccc(N2CCNCC2)cc1O]

### Step 1: tert-Butyl 4-(4-formyl-3-hydroxyphenyl)piperazine-1-carboxylate

To a solution of 5-fluoro-2-hydroxy-benzaldehyde (200 mg, 1.43 mmol) and tert-butyl piperazine-1-carboxylate (266 mg, 1.43 mmol) in DMSO (2 mL) was added DIEA (746 μL, 4.28 mmol). The mixture was degassed, purged with N₂ three times, and stirred at 100 °C for 16 hr under N₂. The reaction mixture was quenched with H₂O (20 mL) at 25 °C and extracted with EtOAc (10 mL×3). The combined organic layers were washed with brine (25 mL×2), dried over Na₂SO₄, filtered, and concentrated under reduced pressure to give a residue. The residue was purified by silica gel column chromatography (Eluent of 1-25% EtOAc in petroleum ether) to give tert-butyl 4-(3-formyl-4-hydroxy-phenyl)piperazine-1-carboxylate (197 mg, yield: 45%). MS: m/z=307.1 [M+H]⁺. ¹H NMR (400 MHz, Chloroform-d) 11.48 (s, 1H), 9.58 (s, 1H), 7.34 (d, J=8.8 Hz, 1H), 6.44 (dd, J=8.8, 2.4 Hz, 1H), 6.25 (d, J=2.4 Hz, 1H), 3.60-3.53 (m, 4H), 3.44-3.37 (m, 4H), 1.48 (s, 9H).

### Step 2: 2-Hydroxy-4-piperazin-1-yl-benzaldehyde

To a solution of tert-butyl 4-(3-formyl-4-hydroxy-phenyl)piperazine-1-carboxylate (190 mg, 621 μmol) in 1,4-dioxane (1 mL) was added HCl in 1,4-dioxane (4M, 2 mL). The mixture was degassed and purged with N₂ three times and stirred at 25 °C for 2 hr under N₂. The reaction mixture was concentrated under reduced pressure to give 2-hydroxy-4-piperazin-1-yl-benzaldehyde (Intermediate 6, 146 mg HCl salt, yield: 98%) which was used directly to the next step without further purification. MS: m/z=207.0 [M+H]⁺.

### Intermediate 7: 2-Hydroxy-5-(piperazin-1-yl)benzaldehyde

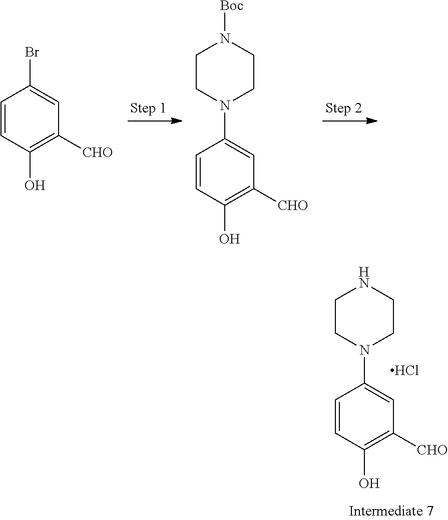 [SMILES: O=Cc1cc(Br)ccc1O ; CC(C)(C)OC(=O)N1CCN(c2ccc(O)c(C=O)c2)CC1 ; O=Cc1cc(N2CCNCC2)ccc1O ; Cl]

### Step 1: tert-Butyl 4-(3-formyl-4-hydroxyphenyl)piperazine-1-carboxylate

To a solution of Pd(OAc)₂ (558 mg, 2.49 mmol) in toluene (10 mL) was added t-Bu₃P (5.03 g, 10% in toluene) and stirred at 25 °C for 10 min. To the resulting mixture were added t-BuONa (5.26 g, 54.7 mmol), 5-bromo-2-hydroxy-benzaldehyde (5 g, 24.9 mmol) and tert-butyl piperazine-1-carboxylate (13.9 g, 74.6 mmol). The mixture was degassed and purged with N₂ three times and stirred at 80 °C for 16 hr under N₂. The reaction mixture was diluted with EtOAc (60 mL) and stirred for 10 min at 25 °C Then the mixture was filtered through celite, the filtrate was washed with brine (50 mL×2), dried over Na₂SO₄, filtered and concentrated under reduced pressure to give a residue. The residue was purified by column chromatography (Eluent of 1-25% EtOAc in petroleum ether), tert-butyl 4-(3-formyl-4-hydroxy-phenyl)piperazine-1-carboxylate (952 mg, yield: 12.2%). MS: m/z=251.0 [M+H-t-Bu]⁻, 207.1 [M+H-Boc]⁺. ¹H NMR (400 MHz, Chloroform-d) 10.66 (s, 1H), 9.86 (s, 1H), 7.25-7.22 (m, 1H), 7.04 (d, J=2.8 Hz, 1H), 6.95 (d, J=8.8 Hz, 1H), 3.62-3.57 (m, 4H), 3.05-3.00 (m, 4H), 1.49 (s, 9H).

### Step 2: 2-Hydroxy-5-(piperazin-1-yl)benzaldehyde

To a solution of tert-butyl 4-(3-formyl-4-hydroxy-phenyl)piperazine-1-carboxylate (400 mg, 1.31 mmol) in 1,4-dioxane (2 mL) was added 4 mL HCl/1,4-dioxane (4 M). The mixture was degassed, purged with N₂ three times and stirred at 25 °C for 1 hr under N₂. The reaction mixture was filtered and the filtered cake was concentrated under reduced pressure to give 2-hydroxy-5-(piperazin-1-yl)benzaldehyde (Intermediate 7, 320 mg, HCl salt). MS: m/z=207.2 [M+H]⁺. 1H NMR (400 MHz, Dimethylsulfoxide-d₆) 10.24 (s, 1H), 9.84 (br s, 1H), 9.37 (br s, 2H), 7.33 (dd, J=9.2, 3.2 Hz, 1H), 7.21 (d, J=3.2 Hz, 1H), 7.02 (d, J=9.2 Hz, 1H), 3.31-3.26 (m, 4H), 3.26-3.17 (m, 4H).

### Intermediate 8: 3-(3-(4-((4-(Cyclopropylamino)piperidin-1-yl)methyl)phenyl)-5-phenyl-3H-imidazo[4,5-b]pyridin-2-yl)pyridin-2-amine

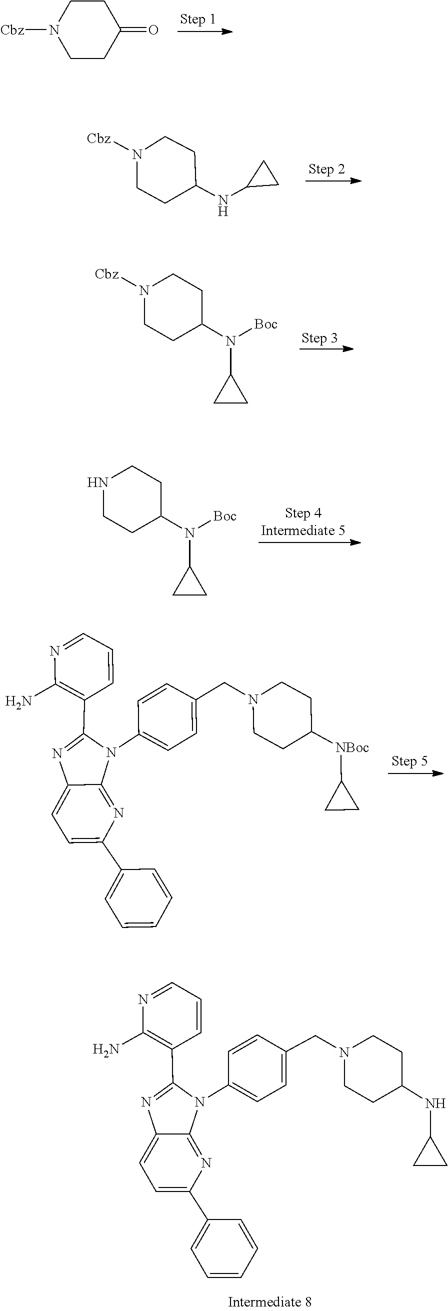 [SMILES: O=C1CCN(C(=O)OCc2ccccc2)CC1 ; O=C(OCc1ccccc1)N1CCC(NC2CC2)CC1 ; CC(C)(C)OC(=O)N(C1CC1)C1CCN(C(=O)OCc2ccccc2)CC1 ; CC(C)(C)OC(=O)N(C1CCNCC1)C1CC1 ; CC(C)(C)OC(=O)N(C1CC1)C1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCC(NC3CC3)CC2)cc1]

### Step 1: Benzyl 4-(cyclopropylamino)piperidine-1-carboxylate

To a solution of benzyl 4-oxopiperidine-1-carboxylate (500 mg, 2.14 mmol) in CH₂C12 (5 mL) were added cyclopropanamine (184 mg, 3.22 mmol, HCl salt) and AcOH (193 mg, 3.22 mmol). The mixture was stirred at 25 °C for 1 hr and then NaBH(OAc)₃ (681 mg, 3.22 mmol) was added. The resulting mixture was stirred at 25 °C for 16 hr. The reaction mixture was quenched with H₂O (10 mL) at 25 °C, diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. Benzyl 4-(cyclopropylamino)piperidine-1-carboxylate (580 mg, yield: 93%) was obtained as a yellow oil. MS: m/z=275.1 [M+H]⁺. ¹H NMR (400 MHz, Chloroform-d) δ 7.42-7.27 (m, 5H), 5.12 (s, 2H), 4.15-4.03 (m, 2H), 2.96-2.85 (m, 2H), 2.82-2.71 (m, 1H), 2.17-2.09 (m, 1H), 1.97-1.88 (m, 2H), 1.76-1.65 (m, 2H), 1.32-1.23 (m, 2H), 0.48-0.42 (m, 2H), 0.36-0.30 (m, 2H).

### Step 2: Benzyl 4-((tert-butoxycarbonyl)(cyclopropyl)amino)piperidine-1-carboxylate

To a solution of benzyl 4-(cyclopropylamino)piperidine-1-carboxylate (580 mg, 2.11 mmol) in THF (10 mL) and H₂O (5 mL) were added Na₂CO₃ (672 mg, 6.34 mmol) and (Boc)₂O (554 mg, 2.54 mmol). The mixture was stirred at 25 °C for 16 hr. The reaction mixture was quenched with H₂O (5 mL) at 25 °C, diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. Benzyl 4-((tert-butoxycarbonyl)(cyclopropyl)amino)piperidine-1-carboxylate (800 mg, yield: 91%) was obtained as a yellow oil. MS: m/z=397.2 [M+Na]⁺. ¹H NMR (400 MHz, Chloroform-d) δ 7.45-7.27 (m, 5H), 5.12 (s, 2H), 4.35-4.17 (m, 2H), 3.84-3.72 (m, 1H), 2.86-2.71 (m, 2H), 2.36-2.25 (m, 1H), 1.97-1.82 (m, 2H), 1.76-1.66 (m, 2H), 1.52 (s, 9H), 0.79-0.71 (m, 2H), 0.68-0.59 (m, 2H).

### Step 3: tert-Butyl cyclopropyl(piperidin-4-yl)carbamate

To a solution of benzyl 4-((tert-butoxycarbonyl)(cyclopropyl)amino)piperidine-1-carboxylate (500 mg, 1.34 mmol) in MeOH (10 mL) was added Pd/C (150 mg, 10% purity) under N₂ atmosphere. The mixture was purged with H₂ three times and stirred at 40 °C under H₂ atmosphere (40 Psi) for 16 hr. The mixture was filtered and washed with MeOH (10 mL×2). The filtrate was concentrated to give tert-butyl cyclopropyl(piperidin-4-yl)carbamate (280 mg, yield: 83%) as a white oil. ¹H NMR (400 MHz, Chloroform-d) δ 3.77-3.68 (m, 1H), 3.13-3.07 (m, 2H), 2.67-2.57 (m, 2H), 2.36-2.28 (m, 1H), 1.93-1.82 (m, 2H), 1.74-1.67 (m, 2H), 1.63-1.55 (m, 1H), 1.45 (s, 9H), 0.76-0.71 (m, 2H), 0.68-0.62 (m, 2H).

### Step 4: tert-Butyl (1-(4-(2-(2-aminopyridin-3-yl)-5-phenyl-3H-imidazo[4,5-b]pyridin-3-yl)benzyl)piperidin-4-yl)(cyclopropyl)carbamate

To a solution of Intermediate 5 (300 mg, 728 μmol) in DMF (10 mL) were added K₂CO₃ (302 mg, 2.19 mmol), NaI (10.9 mg, 72.8 μmol) and tert-butyl cyclopropyl(piperidin-4-yl)carbamate (350 mg, 1.46 mmol). The mixture was stirred at 25 °C for 16 hr. The reaction mixture was quenched with H₂O (10 mL) at 25 °C, diluted with EtOAc (15 mL) and washed with H₂O (20 mL×3). The combined organic layers were washed with brine (20 mL), dried over Na₂SO₄, filtered, and concentrated under reduced pressure. After purified by silica gel flash chromatography (MeOH in CH₂C12=1% to 5%), tert-butyl (1-(4-(2-(2-aminopyridin-3-yl)-5-phenyl-3H-imidazo[4,5-b]pyridin-3-yl)benzyl)piperidin-4-yl)cyclopropyl)carbamate (400 mg, yield: 83%) was obtained as a yellow solid. MS: m/z=616.3 [M+H]⁺.

### Step 5: 3-(3-(4-((4-(Cyclopropylamino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(cyclopropyl)carbamate (150 mg, 244 μmol) in CH₂Cl₂ (3 mL) was added TFA (27.8 mg, 244 μmol). The mixture was stirred at 25 °C for 16 hr. The pH of the mixture was adjusted to 8 with saturated NaHCO₃. The mixture was diluted with CH₂Cl₂ (15 mL), extracted with CH₂Cl₂ (20 mL×3). The combined organic layers were washed with brine (20 mL), dried over Na₂SO₄, filtered and concentrated under reduced pressure. 3-(3-(4-((4-(Cyclopropylamino)piperidin-1-yl)methyl)phenyl)-5-phenyl-3H-imidazo[4,5-b]pyridin-2-yl)pyridin-2-amine (Intermediate 8, 130 mg, yield: 96%) was obtained as a yellow solid. MS: m/z=516.3 [M+H]⁺. ¹H NMR (400 MHz, Dimethylsulfoxide-d₆) δ 8.27 (d, J=8.4 Hz, 1H), 8.05-7.97 (m, 4H), 7.49-7.42 (m, 6H), 7.41-7.38 (m, 1H), 7.16 (dd, J=7.6, 1.6 Hz, 1H), 7.01 (br s, 2H), 6.38 (dd, J=7.6, 4.8 Hz, 1H), 3.58 (s, 2H), 2.91-2.82 (m, 2H), 2.81-2.71 (m, 1H), 2.13-2.00 (m, 2H), 1.95-1.88 (m, 2H), 1.48-1.37 (m, 2H), 1.29-1.14 (m, 2H), 0.59-0.48 (m, 2H), 0.47-0.34 (m, 2H).

### Intermediate 9: 3-(3-(4-((4-(Ethylamino)piperidin-1-yl)methyl)phenyl)-5-phenyl-3H-imidazo[4,5-b]pyridin-2-yl)pyridin-2-amine

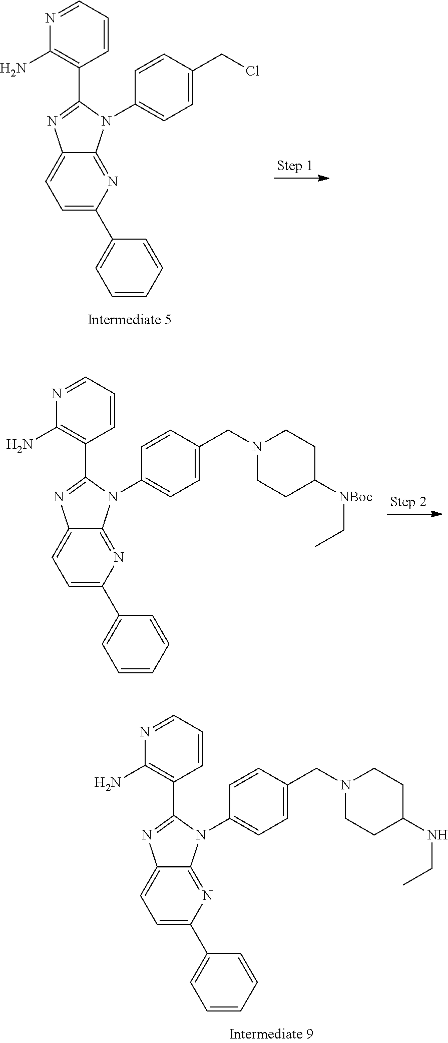 [SMILES: Nc1ncccc1-c1nc2ccc(-c3ccccc3)nc2n1-c1ccc(CCl)cc1 ; CCN(C(=O)OC(C)(C)C)C1CCN(Cc2ccc(-n3c(-c4cccnc4N)nc4ccc(-c5ccccc5)nc43)cc2)CC1 ; CCNC1CCN(Cc2ccc(-n3c(-c4cccnc4N)nc4ccc(-c5ccccc5)nc43)cc2)CC1]

### Step 1: tert-Butyl (1-(4-(2-(2-aminopyridin-3-yl)-5-phenyl-3H-imidazo[4,5-b]pyridin-3-yl)benzyl)piperidin-4-yl)(ethyl)carbamate

To a solution of Intermediate 5 (235 mg, 569 μmol) in DMF (2 mL) were added K₂CO₃ (236 mg, 1.71 mmol), NaI (25.6 mg, 171 μmol) and tert-butyl ethyl(piperidin-4-yl)carbamate (130 mg, 569 μmol). The mixture was stirred at 25 °C for 16 hr. The reaction mixture was poured into H₂O (20 mL). The aqueous phase was extracted with CH₂Cl₂ (20 mL×2). The combined organic layers were washed with brine (20 mL×2), dried over anhydrous Na₂SO₄, filtered and concentrated. tert-Butyl (1-(4-(2-(2-aminopyridin-3-yl)-5-phenyl-3H-imidazo[4,5-b]pyridin-3-yl)benzyl)piperidin-4-yl)(ethyl)carbamate (320 mg, yield: 69%) was obtained as a yellow solid. MS: m/z=604.2 [M+H]⁺.

### Step 2: 3-(3-(4-((4-(Ethylamino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(ethyl)carbamate (320 mg, 530 μmol) in 1,4-dioxane (3.0 mL) was added 4M HCl in 1,4-dioxane (3.0 mL). The mixture was stirred at 25 °C for 0.5 hr. The mixture was concentrated to give 3-(3-(4-((4-(ethylamino)piperidin-1-yl)methyl)phenyl)-5-phenyl-3H-imidazo[4,5-b]pyridin-2-yl)pyridin-2-amine (Intermediate 9, 230 mg HCl salt) as a yellow solid. MS: m/z=504.3. [M+H]⁺. ¹H NMR (400 MHz, Dimethylsulfoxide-d₆) δ 8.27 (d, J=8.0 Hz, 1H), 8.06-7.96 (m, 4H), 7.49-7.38 (m, 7H), 7.15 (dd, J=7.6, 1.6 Hz, 1H), 7.03 (br s, 2H), 6.37 (dd, 1=7.6, 4.8 Hz, 11H), 3.54 (s, 2H), 2.82-2.73 (m, 2H), 2.58-2.52 (m, 2H), 2.40-2.34 (m, 1H), 2.03-1.93 (m, 2H), 1.82-1.74 (m, 2H), 1.32-1.21 (m, 2H), 0.99 (t, J=7.2 Hz, 3H).

### Intermediate 10: tert-Butyl 4-(4-(2-(2-aminopyridin-3-yl)-5-chloro-3H-imidazo[4,5-b]pyridin-3-yl)benzyl)piperazine-1-carboxylate

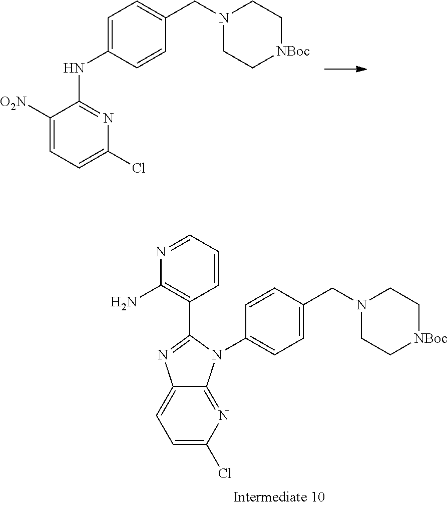 [SMILES: CC(C)(C)OC(=O)N1CCN(Cc2ccc(Nc3nc(Cl)ccc3[N+](=O)[O-])cc2)CC1 ; CC(C)(C)OC(=O)N1CCN(Cc2ccc(-n3c(-c4cccnc4N)nc4ccc(Cl)nc43)cc2)CC1]

To a solution of tert-butyl 4-(4-((6-chloro-3-nitropyridin-2-yl)amino)benzyl)piperazine-1-carboxylate (refer to Intermediate 3 for detail procedures, 21 g, 46.9 mmol) in DMSO (300 mL) were added 2-aminonicotinaldehyde (6.87 g, 56.4 mmol) and Na₂S₂O₄ (28.8 g, 141 mmol). The mixture was stirred at 100 °C for 16 hr. The reaction mixture was quenched with H₂O (300 mL) at 25 °C and extracted with CH₂Cl₂ (500 mL×2). The combined organic layers were washed with H₂O (1000 mL×3), dried over Na₂SO₄, filtered and concentrated under reduced pressure to give a crude product. After purified by silica gel flash chromatography (Eluent of 1~8% MeOH in CH₂Cl₂), tert-butyl 4-(4-(2-(2-aminopyridin-3-yl)-5-chloro-3H-imidazo[4,5-b]pyridin-3-yl)benzyl)piperazine-1-carboxylate (Intermediate 10, 6.3 g, yield: 25%) was obtained as a yellow solid. MS: m/z=520.1 [M+H]⁺. ¹H NMR (400 MHz, Chloroform-d) δ 8.05 (dd, J=4.8, 2.0 Hz, 1H), 8.02 (d, J=8.4 Hz, 1H), 7.49 (d, J=8.4 Hz, 2H), 7.31 (dd, J=8.4, 2.8 Hz, 3H), 7.02 (dd, J=8.0, 2.0 Hz, 1H), 6.62 (br s, 2H), 6.33 (dd, J=8.0, 4.8 Hz, 1H), 3.61 (s, 2H), 3.50-3.45 (m, 4H), 2.49-2.43 (m, 4H), 1.47 (s, 9H).

### Intermediate 11: 3-(2-(2-Aminopyridin-3-yl)-3-(4-(piperazin-1-ylmethyl)phenyl)-3H-imidazo[4,5-b]pyridin-5-yl)pyridin-2(1H)-one

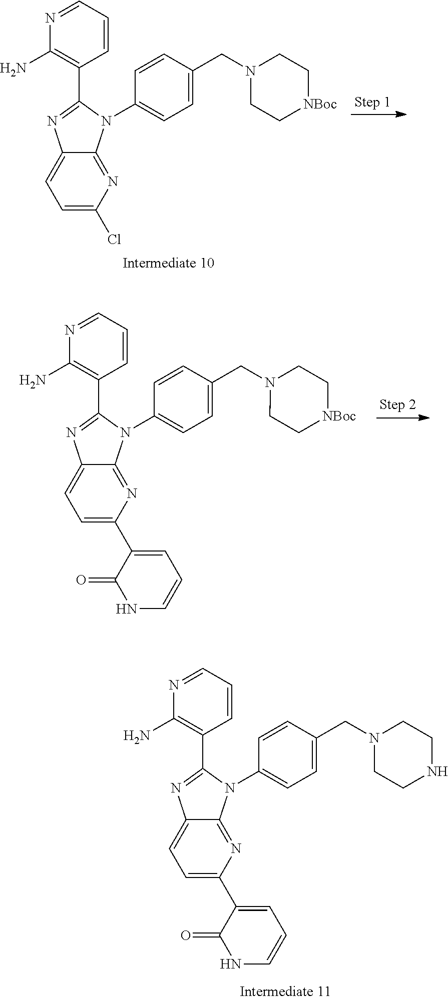 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5ccc[nH]c5=O)nc43)cc2)CC1 ; Nc1ncccc1-c1nc2ccc(-c3ccc[nH]c3=O)nc2n1-c1ccc(CN2CCNCC2)cc1]

### Step 1: tert-Butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate

A mixture of Intermediate 10 (250 mg, 481 μmol), (2-oxo-1,2-dihydropyridin-3-yl)boronic acid (80.1 mg, 577 μmol), Cs₂CO₃ (470 mg, 1.44 mmol), and Pd(dppf)Cl₂ (70.4 mg, 96.2 μmol) in 1,4-dioxane (5 mL) and H₂O (1 mL) was degassed and purged with N₂ three times. The mixture was stirred at 80 °C for 16 hr under N₂ atmosphere. The reaction mixture was filtered at 25 °C, diluted with H₂O (20 mL) and extracted with EtOAc (20 mL×3). The combined organic layers were washed with brine (30 mL), dried over Na₂SO₄, filtered, and concentrated under reduced pressure. After purified by silica gel flash chromatography (Eluent of 1-100% EtOAc in petroleum ether and eluent of 1-10% MeOH in CH₂Cl₂), tert-butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate (200 mg, yield: 58%) was obtained as a brown solid. MS: m/z=579.3 [M+H]⁺.

### Step 2: 3-(2-(2-Aminopyridin-3-yl)-3-(4-(piperazin-1-ylmethyl)phenyl)-3H-imidazo[4,5-b]pyridin-5-yl)pyridin-2(1H)-one

To a solution of tert-butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate (200 mg, 346 μmol) in 1,4-dioxane (3 mL) was added 4 M HCl in 1,4-dioxane (3 mL) at 25 °C The mixture was stirred at 25 °C for 16 hr. The pH of the mixture was adjusted to ~8 with NaHCO₃. The mixture was extracted with CH₂Cl₂ (20 mL×3). The combined organic layers were washed with brine (20 mL), dried over Na₂SO₄, filtered and concentrated under reduced pressure to give 3-(2-(2-aminopyridin-3-yl)-3-(4-(piperazin-1-ylmethyl)phenyl)-3H-imidazo[4,5-b]pyridin-5-yl)pyridin-2(1H)-one (Intermediate 11, 200 mg, yield: 85%) as a yellow solid. MS: m/z=479.2 [M+H]⁺.

### Intermediate 12: 3-(3-(4-(Chloromethyl)phenyl)-3H-imidazo[4,5-b]pyridin-2-yl)pyridin-2-amine

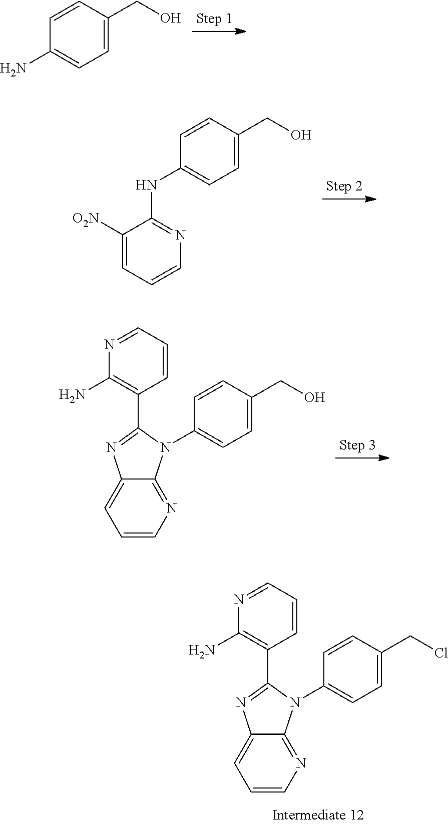

### Step 1: (4-((3-Nitropyridin-2-yl)amino)phenyl)methanol

To a solution of (4-aminophenyl)methanol (10 g, 81.2 mmol) in 1,4-dioxane (150 mL) were added DIEA (31.5 g, 244 mmol) and 2-chloro-3-nitro-pyridine (15.5 g, 97.4 mmol). The mixture was stirred at 100 °C for 16 hr. The reaction mixture was concentrated under reduced pressure. The residue was diluted with H₂O (200 mL) and extracted with EtOAc (200 mL×2). The combined organic layers were washed with brine (500 mL), dried over Na₂SO₄, filtered, and concentrated under reduced pressure. The crude product was purified by silica gel flash chromatography (Eluent of 0~82% EtOAc in petroleum ether) to give (4-((3-nitropyridin-2-yl)amino)phenyl)methanol (15.3 g, yield: 77%) as a yellow solid. MS: m/z=245.8 [M+H]⁺. ¹H NMR (400 MHz, Chloroform-d) δ 10.12 (s, 1H), 8.53 (dd, J=8.4, 2.0 Hz, 1H), 8.48 (dd, J=4.4, 1.6 Hz, 1H), 7.64 (d, J=8.0 Hz, 2H), 7.40 (d, J=8.4 Hz, 2H), 6.84 (dd, J=8.4, 4.8 Hz, 1H), 4.70 (s, 2H).

### Step 2: (4-(2-(2-Aminopyridin-3-yl)-3H-imidazo[4,5-b]pyridin-3-yl)phenyl)methanol

To a solution of 4-((3-nitropyridin-2-yl)amino)phenyl)methanol (10 g, 40.8 mmol) in DMSO (500 mL) were added Na₂S₂O₄ (21.3 g, 122 mmol) and 2-aminopyridine-3-carbaldehyde (5.98 g, 48.9 mmol). The mixture was stirred at 100 °C for 16 hr. The reaction mixture was quenched with H₂O (500 mL) at 20 °C and extracted with EtOAc (500 mL×2). The combined organic layers were washed with brine (500 mL), dried over Na₂SO₄, filtered, and concentrated under reduced pressure. The crude product was purified by silica gel flash chromatography (Eluent of 0~100% EtOAc in petroleum ether) to give (4-(2-(2-aminopyridin-3-yl)-3H-imidazo[4,5-b]pyridin-3-yl)phenyl)methanol (3.1 g, yield: 24%) as a yellow solid. MS: m/z=318.3 [M+H]⁺. ¹H NMR (400 MHz, Chloroform-c) δ 8.39-8.32 (m, 1H), 8.08 (dd, J=8.0, 1.2 Hz, 1H), 8.02 (dd, J=4.8, 1.6 Hz, 1H), 7.52 (d, J=8.4 Hz, 2H), 7.35 (d, J=8.0 Hz, 2H), 7.30-7.27 (m, 1H), 7.12 (dd, J=7.6, 1.6 Hz, 1H), 6.65 (br s, 2H), 6.35 (dd, J=7.6, 4.8 Hz, 1H), 4.76 (s, 2H).

### Step 3: 3-(3-(4-(Chloromethyl)phenyl)-3H-imidazo[4,5-b]pyridin-2-yl)pyridin-2-amine

To a solution of (4-(2-(2-aminopyridin-3-yl)-3H-imidazo[4,5-b]pyridin-3-yl)phenyl)methanol (1.0 g, 3.15 mmol) in CH₂Cl₂ (10 mL) was added SOCl₂ (3.3 g, 27.6 mmol). The mixture was stirred at 40 °C for 1 hr. The reaction mixture was concentrated under reduced pressure to give 3-(3-(4-(chloromethyl)phenyl)-3H-imidazo[4,5-b]pyridin-2-yl)pyridin-2-amine (Intermediate 12, 1.2 g HCl salt) as a gray solid, which was used in the next step without further purification. MS: m/z=336.0 [M+H]⁺. ¹H NMR (400 MHz, Chloroform-d) δ 9.35 (br s, 1H), 8.55 (d, J=8.0 Hz, 1H), 8.28 (d, J=7.6 Hz, 1H), 7.91-7.83 (m, 1H), 7.68 (d, J=8.0 Hz, 2H), 7.64-7.59 (m, 1H), 7.54-7.50 (m, 1H), 7.46 (d, J=8.0 Hz, 2H), 7.30-7.27 (m, 2H), 6.70-6.60 (m, 1H), 4.70 (s, 2H).

### Intermediate 13: 4-Bromo-3-methoxy-N-methylaniline

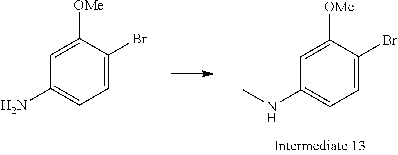

To a solution of 4-bromo-3-methoxyaniline (5 g, 24.8 mmol) and K₂CO₃ (10.3 g, 74.2 mmol) in MeCN (50 mL) was added MeI (3.51 g, 24.8 mmol) at 0 °C The mixture was stirred at 25 °C for 16 hr. The reaction mixture was quenched with aqueous NH₄Cl (50 mL) at 25 °C and extracted with EtOAc (100 mL). The combined organic layers were washed with brine (200 mL×2), dried over Na₂SO₄, filtered, and concentrated under reduced pressure. After purified by silica gel flash chromatography (Eluent of 10%-30% EtOAc in petroleum ether), 4-bromo-3-methoxy-N-methylaniline (Intermediate 13, 1.2 g, yield: 20%) was obtained as a yellow oil. MS: m/z=216.0, 218.0 [M+H]⁺. ¹H NMR (400 MHz, Chloroform-d) δ 7.24 (d, J=4.0 Hz, 1H), 6.16 (d, J=2.4 Hz, 1H), 6.11 (dd, J=8.4, 2.4 Hz, 1H), 3.82 (s, 3H), 2.81 (s, 3H).

### Intermediate 14: 1-(3-Bromo-2-methoxyphenyl)piperazine

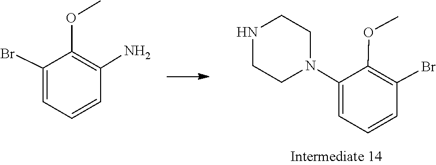

A mixture of 3-bromo-2-methoxyaniline (2.0 g, 9.90 mmol) and bis(2-chloroethyl)amine (1.94 g, 10.9 mmol, HCl salt) in n-BuOH (20 mL) was added Na₂CO₃ (1.05 g, 9.90 mmol). The mixture was stirred at 120 °C for 24 hr under N₂ atmosphere. The reaction mixture was filtered to give 1-(3-bromo-2-methoxyphenyl)piperazine (Intermediate 14, 2.2 g, yield: 82%) as a yellow solid. MS: m/z=270.9, 272.9[M+H]⁺.

### Intermediate 15: 2-fluoro-6-hydroxy-4-(piperazin-1-yl)benzaldehyde

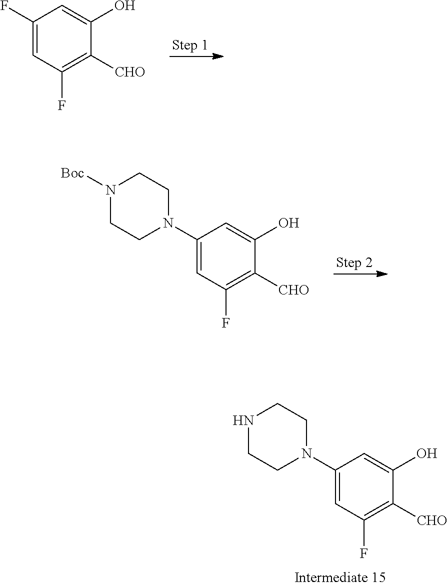 [SMILES: O=Cc1c(O)cc(F)cc1F ; CC(C)(C)OC(=O)N1CCN(c2cc(O)c(C=O)c(F)c2)CC1 ; O=Cc1c(O)cc(N2CCNCC2)cc1F]

### Step 1: tert-Butyl 4-(3-fluoro-4-formyl-5-hydroxyphenyl)piperazine-1-carboxylate

To a solution of 2,4-difluoro-6-hydroxybenzaldehyde (200 mg, 1.27 mmol) and tert-butyl piperazine-1-carboxylate (563 mg, 2.53 mmol) in DMSO (5 mL) was added DIEA (817 mg, 6.33 mmol). The mixture was stirred at 100 °C for 2 hr. The reaction mixture was diluted with water (20 mL) and extracted with EtOAc (3×30 mL). The combined organic layers were washed with brine (30 mL), dried over Na₂SO₄, filtered, and concentrated under reduced pressure. After purified by silica gel flash chromatography (EtOAc in petroleum ether=0 to 25%), tert-butyl 4-(3-fluoro-4-formyl-5-hydroxyphenyl)piperazine-1-carboxylate (300 mg, yield: 69.5%) was obtained as a yellow solid. ¹H NMR (400 MHz, Dimethylsulfoxide-d₆) δ 11.77 (s, 1H), 9.82 (s, 1H), 6.42 (dd, J=15.2, 2.0 Hz, 1H), 6.14 (d, J=1.6 Hz, 1H), 3.48-3.40 (m, 8H), 1.42 (s, 9H).

### Step 2: 2-Fluoro-6-hydroxy-4-(piperazin-1-yl)benzaldehyde

To a solution of tert-butyl 4-(3-fluoro-4-formyl-5-hydroxyphenyl)piperazine-1-carboxylate (300 mg, 925 μmol) in CH₂Cl₂ (1 mL) was added HCl in 1,4-dioxane (4 M, 231 μL). The mixture was stirred at 25 °C for 1 hr. The mixture was concentrated under reduced pressure to give 2-fluoro-6-hydroxy-4-(piperazin-1-yl)benzaldehyde (Intermediate 15, 200 mg, crude, HCl salt) as a yellow solid which was used in the next step. ¹H NMR (400 MHz, Dimethylsulfoxide-d₆) δ 11.72 (s, 1H), 9.86 (s, 1H), 9.55 (br s, 2H), 6.50 (dd, J=15.2, 2.4 Hz, 1H), 6.25 (s, 1H), 3.68 (t, J=0.5.2 Hz, 4H).

### Intermediate 16: 5-Fluoro-2-hydroxy-4-(piperazin-1-yl)benzaldehyde

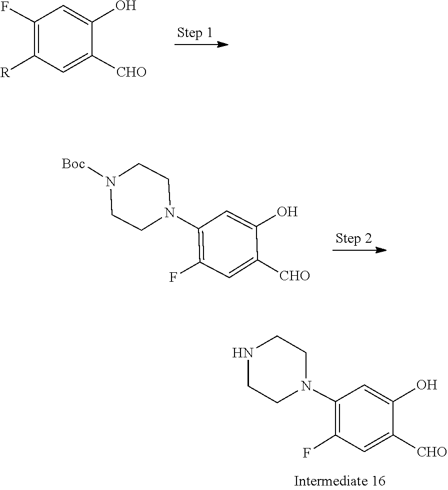

Following the general procedures described in Example 15, the reactions were carried out using corresponding starting materials. 5-fluoro-2-hydroxy-4-(piperazin-1-yl)benzaldehyde (Intermediate 16, 160 mg, crude, HCl) was obtained as a yellow solid, which was used in the next step without further purification.

### Intermediate 17: 3-Fluoro-2-hydroxy-4-(piperazin-1-yl)benzaldehyde

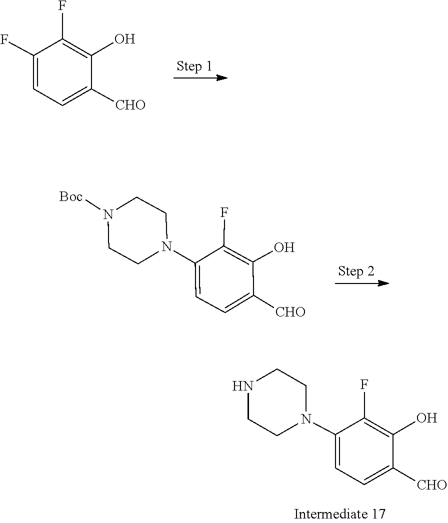 [SMILES: O=Cc1ccc(F)c(F)c1O ; CC(C)(C)OC(=O)N1CCN(c2ccc(C=O)c(O)c2F)CC1 ; O=Cc1ccc(N2CCNCC2)c(F)c1O]

Following the general procedures described in Example 15, the reactions were carried out using corresponding starting materials. 3-Fluoro-2-hydroxy-4-(piperazin-1-yl)benzaldehyde (Intermediate 17, 160 mg, crude, HCl) was obtained as a yellow solid, which was used in the next step without further purification. MS: m/z=225.0 [M+H]⁺.

### Intermediate 18: 5-Bromo-2-hydroxy-4-(piperazin-1-yl)benzaldehyde

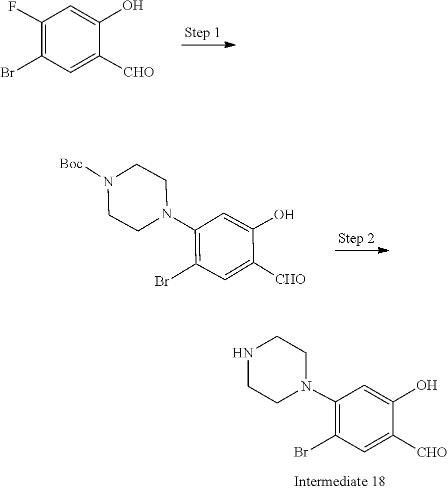 [SMILES: O=Cc1cc(Br)c(F)cc1O ; CC(C)(C)OC(=O)N1CCN(c2cc(O)c(C=O)cc2Br)CC1 ; O=Cc1cc(Br)c(N2CCNCC2)cc1O]

Following the general procedures described in Example 15, the reactions were carried out using corresponding starting materials. 5-Bromo-2-hydroxy-4-(piperazin-1-yl)benzaldehyde (Intermediate 18, 120 mg, HCl salt) was obtained as a yellow solid. MS: m/z=285.0, 287.0 [M+H]⁺.

### Intermediate 19: 2-Hydroxy-4-(piperazin-1-yl)-5-vinylbenzaldehyde

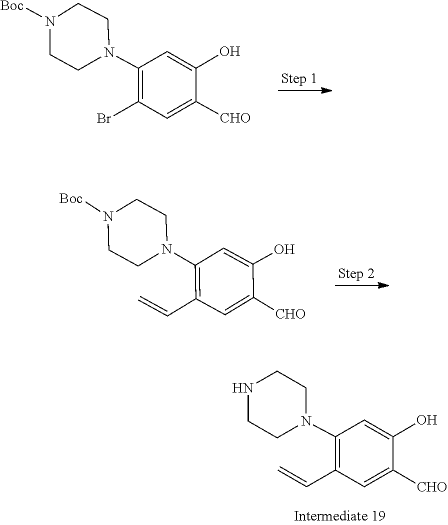 [SMILES: CC(C)(C)OC(=O)N1CCN(c2cc(O)c(C=O)cc2Br)CC1 ; C=Cc1cc(C=O)c(O)cc1N1CCN(C(=O)OC(C)(C)C)CC1 ; C=Cc1cc(C=O)c(O)cc1N1CCNCC1]

### Step 1: tert-Butyl 4-(4-formyl-5-hydroxy-2-vinylphenyl)piperazine-1-carboxylate

A mixture of tert-butyl 4-(2-bromo-4-formyl-5-hydroxyphenyl)piperazine-1-carboxylate (refer to Intermediate 18 for detail procedures, 500 mg, 1.30 mmol), 4,4,5,5-tetramethyl-2-vinyl-1,3,2-dioxaborolane (239 mg, 1.56 mmol), Pd(dppf)Cl₂ (94.9 mg, 129 μmol), Cs₂CO₃ (1.27 g, 3.89 mmol) in 1,4-dioxane (5 mL) and H₂O (0.5 mL) was degassed and purged with N₂ three times, and the mixture was stirred at 80 °C for 12 hr under N₂ atmosphere. The reaction mixture was diluted with water (20 mL) and extracted with CH₂Cl₂ (3×30 mL). The combined organic layers were dried over Na₂SO₄, filtered and concentrated under reduced pressure. After purified by silica gel flash chromatography (Eluent of 0~4%, EtOAc in petroleum ether), tert-butyl 4-(4-formyl-5-hydroxy-2-vinylphenyl)piperazine-1-carboxylate (120 mg, yield: 26%) was obtained as a yellow solid. MS: m/z=277.1 [M-56]⁺. ¹H NMR (400 MHz, Chloroform-d) δ 11.26 (s, 1H), 9.77 (s, 1H), 7.56 (s, 1H), 6.75 (dd, 0.1=17.6, 10.8 Hz, 1H), 6.45 (s, 1H), 5.65 (d, J=17.6 Hz, 1H), 5.24 (d, J=10.8 Hz, 1H), 3.62-3.53 (m, 4H), 3.09-2.99 (m, 4H), 1.49 (s, 9H).

### Step 2: 2-Hydroxy-4-(piperazin-1-yl)-5-vinylbenzaldehyde

To a solution of tert-butyl 4-(4-formyl-5-hydroxy-2-vinylphenyl)piperazine-1-carboxylate (120 mg, 361 μmol) in CH₂Cl₂ (1 mL) was added HCl in 1,4-dioxane (4 M, 90.2 μL). The mixture was stirred at 25 °C for 0.5 hr. The mixture was concentrated under reduced pressure to give 2-hydroxy-4-(piperazin-1-yl)-5-vinylbenzaldehyde (Intermediate 19, 90 mg, HCl) as a green solid. MS: m/z=233.1 [M+H]⁺.

### Intermediate 20: 3-(5-(3,6-Dihydro-2H-pyran-4-yl)-3-(4-(piperazin-1-ylmethyl)phenyl)-3H-imidazo[4,5-b]pyridin-2-yl)pyridin-2-amine

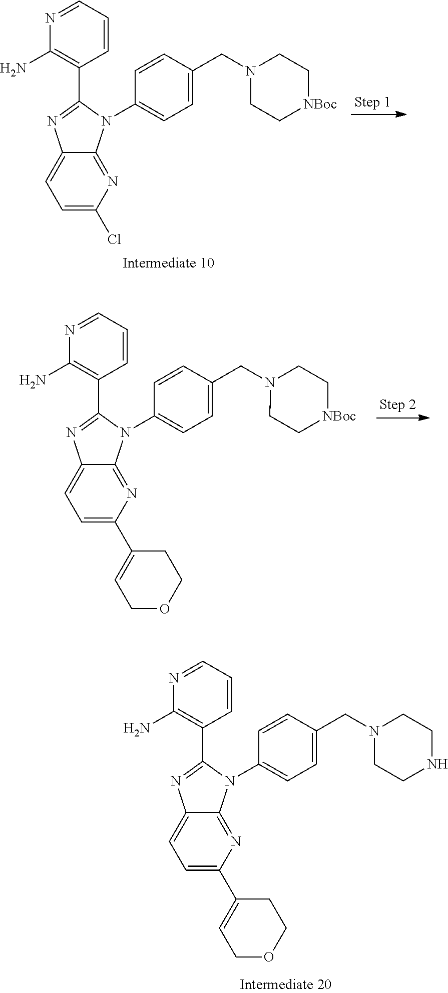 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(C5=CCOCC5)nc43)cc2)CC1 ; Nc1ncccc1-c1nc2ccc(C3=CCOCC3)nc2n1-c1ccc(CN2CCNCC2)cc1]

### Step 1: tert-Butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate

To a solution of Intermediate 10 (200 mg, 385 μmol) in 1,4-dioxane (5 mL) and H₂O (1 mL) were added 2-(3,6-dihydro-2H-pyran-4-yl)-4,4,5,5-tetramethyl-1,3,2-dioxaborolane (121 mg, 577 μmol), K₂CO₃ (159 mg, 1.15 mmol) and Pd(dppf)Cl₂ (28.1 mg, 38.5 μmol) at 25 °C under N₂. This mixture was stirred at 100 °C for 5 hr. The mixture was diluted with water (5 mL) and extracted with EtOAc (20 mL×3). The combined organic layers were washed with brine (30 mL), dried over anhydrous Na₂SO₄, filtered and concentrated. The residue was purified by prep-TLC (EtOAc:petroleum ether=2:1) to give tert-butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate (45 mg, yield: 50%) as an off-white solid. MS: m/z=568.3 [M+H]⁺. ¹H NMR (400 MHz, Methanol-d₄) δ 8.08 (d, J=8.4 Hz, 1H), 7.97 (dd, J=4.8, 1.6 Hz, 1H), 7.62 (d, J=8.4 Hz, 1H), 7.51 (d, J=8.0 Hz, 2H), 7.39 (d, J=8.4 Hz, 2H), 7.29 (dd, J=8.0, 2.0 Hz, 1H), 6.72-6.65 (m, 1H), 6.47 (dd, J=8.0, 5.2 Hz, 1H), 4.36-4.28 (m, 2H), 3.90 (t, J=5.6 Hz, 2H), 3.62 (s, 2H), 3.46-3.45 (m, 4H), 2.67-2.60 (m, 2H), 2.46 (t, J=4.8 Hz, 4H), 1.46 (s, 9H).

### Step 2: 3-(5-(3,6-Dihydro-2H-pyran-4-yl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate (45.0 mg, 79.3 μmol) in 1,4-dioxane (1 mL) was added HCl/1,4-dioxane (4 M, 19.8 μL) at 25 °C This mixture was stirred at 25 °C for 3 hr. The mixture was filtrated and concentrated. 3-(5-(3,6-Dihydro-2H-pyran-4-yl)-3-(4-(piperazin-1-ylmethyl)phenyl)-3H-imidazo[4,5-b]pyridin-2-yl)pyridin-2-amine (Intermediate 20, 20.8 mg, HCl salt, yield: 52%) was obtained as a yellow solid, which was used in the next step without purification. MS: m/z=468.2 [M+H]⁺. ¹H NMR (400 MHz, Methanol-d₄) δ 8.21 (d, J=8.4 Hz, 1H), 8.01 (dd, J=6.4, 1.2 Hz, 1H), 7.93-7.83 (m, 3H), 7.75-7.62 (m, 3H), 6.96-6.87 (m, 1H), 6.75-6.74 (m, 1H), 4.62 (s, 2H), 4.39-4.29 (m, 2H), 3.90 (t, J=5.2 Hz, 2H), 3.69-3.68 (m, 8H), 2.68-2.57 (m, 2H).

### Intermediate 21: 3-(3-(4-(Chloromethyl)phenyl)-5-morpholino-3H-imidazo[4,5-b]pyridin-2-yl)pyridin-2-amine

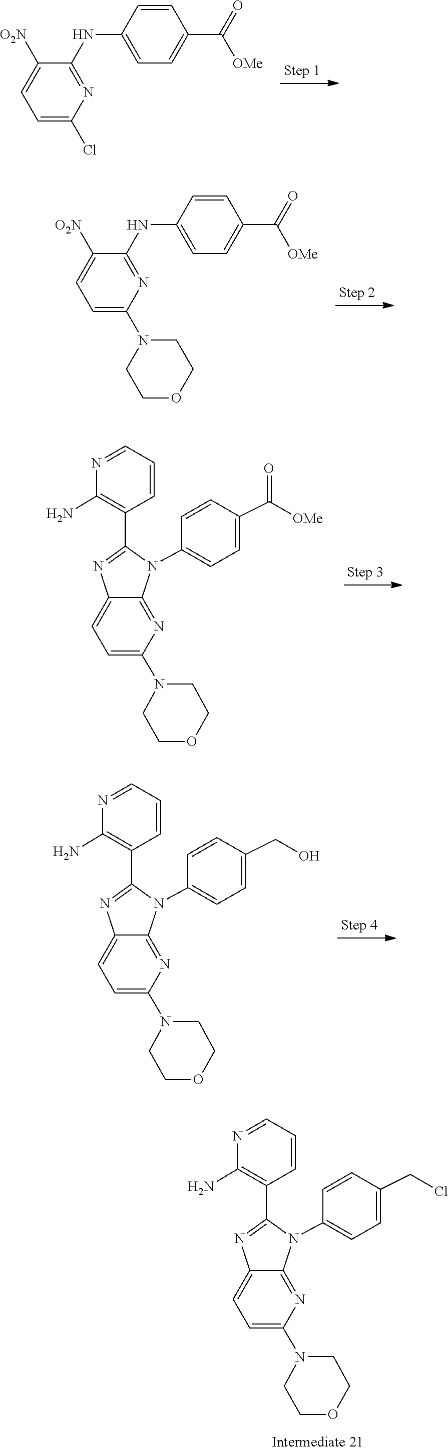 [SMILES: COC(=O)c1ccc(Nc2nc(Cl)ccc2[N+](=O)[O-])cc1 ; COC(=O)c1ccc(Nc2nc(N3CCOCC3)ccc2[N+](=O)[O-])cc1 ; COC(=O)c1ccc(-n2c(-c3cccnc3N)nc3ccc(N4CCOCC4)nc32)cc1 ; Nc1ncccc1-c1nc2ccc(N3CCOCC3)nc2n1-c1ccc(CO)cc1 ; Nc1ncccc1-c1nc2ccc(N3CCOCC3)nc2n1-c1ccc(CCl)cc1]

### Step 1: Methyl 4-((6-morpholino-3-nitropyridin-2-yl)amino)benzoate

A mixture of methyl 4-((6-chloro-3-nitropyridin-2-yl)amino)benzoate (refer to Intermediate 5 for detail procedures, 2.5 g, 8.13 mmol), morpholine (849 mg, 9.75 mmol), DIEA (2.10 g, 16.2 mmol) in CH₃CN (30 mL) was stirred at 90 °C for 2 hr. The reaction mixture was filtered and the filter cake was dried under reduced pressure to give methyl 4-((6-morpholino-3-nitropyridin-2-yl)amino)benzoate (2.94 g, yield: 92%) as a yellow solid. MS: m/z=359.0 [M+H]⁺. ¹H NMR (400 MHz, Dimethylsulfoxide-d₆) 10.74 (s, 1H), 8.27 (d, J=9.6 Hz, 1H), 7.98-7.94 (m, 2H), 7.83-7.79 (m, 2H), 6.58 (d, J=9.6 Hz, 1H), 3.84 (s, 3H), 3.75-3.65 (m, 8H).

### Step 2: Methyl 4-(2-(2-aminopyridin-3-yl)-5-morpholino-3H-imidazo[4,5-b]pyridin-3-yl)benzoate

A mixture of methyl 4-((6-morpholino-3-nitropyridin-2-yl)amino)benzoate (2.94 g, 8.20 mmol), 2-aminonicotinaldehyde (1.10 g, 9.02 mmol), and Na₂S₂O₄ (5.71 g, 32.8 mmol) in DMSO (30 mL) was stirred at 100 °C for 16 hr. The reaction mixture was cooled to room temperature and the pH was adjusted to about 9 with sat. NaHCO₃. The mixture was diluted with H₂O (100 mL) and extracted with CH₂Cl₂ (100 mL×3). The combined organic layers were washed with brine (100 mL), dried over Na₂SO₄, filtered, and concentrated under reduced pressure. The crude was triturated with EtOAc (100 mL) at 20 °C for 30 min to give methyl 4-(2-(2-aminopyridin-3-yl)-5-morpholino-3H-imidazo[4,5-b]pyridin-3-yl)benzoate (3.5 g, yield: 83%) as a green solid. MS: m/z=431.1 [M+H]⁺. ¹H NMR (400 MHz, Dimethylsulfoxide-d₆) δ 8.07 (d, J=8.4 Hz, 2H), 8.03-7.97 (m, 2H), 7.55 (d, J=8.8 Hz, 2H), 7.32-7.13 (m, 3H), 6.93 (d, J=8.8 Hz, 1H), 6.51-6.44 (m, 11H), 3.88 (s, 31H), 3.70-3.65 (m, 4H), 3.44-3.38 (m, 4H).

### Step 3: (4-(2-(2-Aminopyridin-3-yl)-5-morpholino-3H-imidazo[4,5-b]pyridin-3-yl)phenyl)methanol

A mixture of methyl 4-(2-(2-aminopyridin-3-yl)-5-morpholino-3H-imidazo[4,5-b]pyridin-3-yl)benzoate (3.5 g, 8.13 mmol) in THF (100 mL) was added LiAlH₄ (2.5 M, 3.90 mL) dropwise at 0 °C and stirred at 20 °C for 1 hr. The reaction mixture was quenched with Na₂SO₄·10H₂O (5 g) at 0 °C, filtered and concentrated under reduced pressure to give (4-(2-(2-aminopyridin-3-yl)-5-morpholino-3H-imidazo[4,5-b]pyridin-3-yl)phenyl)methanol (2.4 g, yield: 63%) as a green solid. MS: m/z=403.1 [M+H]⁺.

### Step 4: 3-(3-(4-(Chloromethyl)phenyl)-5-morpholino-3H-imidazo[4,5-b]pyridin-2-yl)pyridin-2-amine

To a mixture of (4-(2-(2-aminopyridin-3-yl)-5-morpholino-3H-imidazo[4,5-b]pyridin-3yl)phenyl)methanol (2.4 g, 5.96 mmol) in CH₂Cl₂ (16 mL) was added SOCl₂ (4.26 g, 35.7 mmol). The mixture was stirred at 40 °C for 1 hr. The reaction mixture was concentrated under reduced pressure to give 3-(3-(4-(chloromethyl)phenyl)-5-morpholino-3H-imidazo[4,5-b]pyridin-2-yl)pyridin-2-amine (Intermediate 21, 2.6 g, yield: 64%) as a green solid. MS: m/z=421.0 [M+H]⁺.

### Intermediate 22: 3-(5-Morpholino-3-(4-(piperazin-1-ylmethyl)phenyl)-3H-imidazo[4,5-b]pyridin-2-yl)pyridin-2-amine

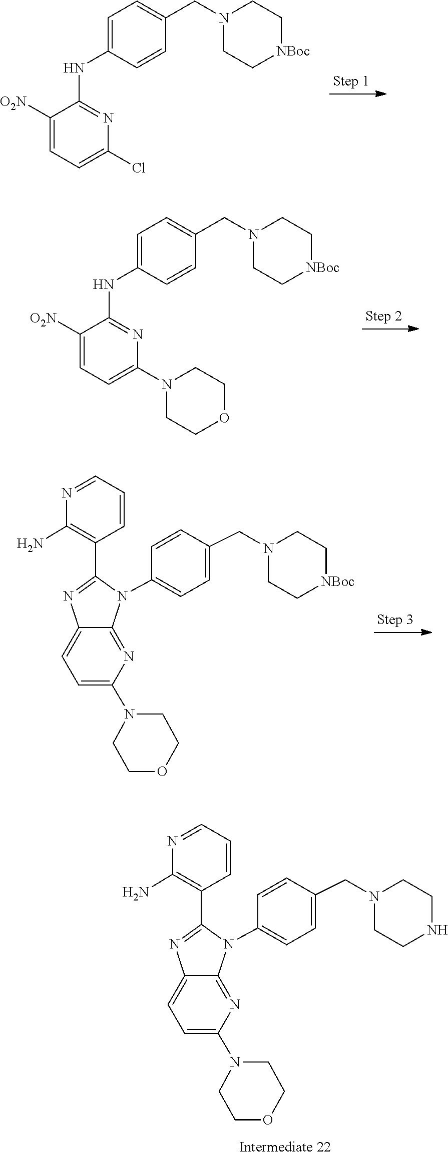 [SMILES: CC(C)(C)OC(=O)N1CCN(Cc2ccc(Nc3nc(Cl)ccc3[N+](=O)[O-])cc2)CC1 ; CC(C)(C)OC(=O)N1CCN(Cc2ccc(Nc3nc(N4CCOCC4)ccc3[N+](=O)[O-])cc2)CC1 ; CC(C)(C)OC(=O)N1CCN(Cc2ccc(-n3c(-c4cccnc4N)nc4ccc(N5CCOCC5)nc43)cc2)CC1 ; Nc1ncccc1-c1nc2ccc(N3CCOCC3)nc2n1-c1ccc(CN2CCNCC2)cc1]

### Step 1: tert-Butyl 4-(4-((6-morpholino-3-nitropyridin-2-yl)amino)benzyl)piperazine-1-carboxylate

To a solution of tert-butyl 4-(4-((6-chloro-3-nitropyridin-2-yl)amino)benzyl)piperazine-1-carboxylate (refer to Intermediate 5 for detail procedures, 300 mg, 670 μmol) and morpholine (117 mg, 1.34 mmol) in CH₃CN (4 mL) was added DIEA (173 mg, 1.34 mmol). The mixture was stirred at 90 °C for 2 hr. The reaction mixture was quenched with H₂O (10 mL) at 25 °C and extracted with CH₂Cl₂ (15 mL×2). The combined organic layers were washed with brine (15 mL), dried over Na₂SO₄, filtered and concentrated under reduced pressure to give tert-butyl 4-(4-((6-morpholino-3-nitropyridin-2-yl)amino)benzyl)piperazine-1-carboxylate (330 mg, yield: 93%) as a yellow solid. MS: m/z=499.2 [M+H]⁺. ¹H NMR (400 MHz, Chloroform-d) δ 10.71 (s, 1H), 8.32 (d, J=9.2 Hz, 1H), 7.53 (d, J=8.0 Hz, 2H), 7.30 (d, J=8.0 Hz, 2H), 6.13 (d, J=9.6 Hz, 1H), 3.79-3.75 (m, 4H), 3.73-3.67 (m, 4H), 3.50 (s, 2H), 3.46-3.40 (m, 4H), 2.44-2.35 (m, 4H), 1.45 (s, 9H).

### Step 2: tert-Butyl 4-(4-(2-(2-aminopyridin-3-yl)-5-morpholino-3H-imidazo[4,5-b]pyridin-3-yl)benzyl)piperazine-1-carboxylate

To a solution of tert-butyl 4-(4-((6-morpholino-3-nitropyridin-2-yl)amino)benzyl)piperazine-1-carboxylate (330 mg, 662 μmol) in DMSO (10 mL) were added 2-aminonicotinaldehyde (97 mg, 794 μmol) and Na₂S₂O₄ (542 mg, 2.65 mmol). The mixture was stirred at 100 °C for 2 hr. The reaction mixture was quenched with H₂O (20 mL) at 25 °C and extracted with CH₂Cl₂ (50 mL×3). The combined organic layers were washed with brine (50 mL), dried over Na₂SO₄, filtered and concentrated under reduced pressure to give the crude product. The crude was purified by silica gel flash chromatography (Eluent of 1~5% MeOH in CH₂Cl₂) to give tert-butyl 4-(4-(2-(2-aminopyridin-3-yl)-5-morpholino-3H-imidazo[4,5-b]pyridin-3-yl)benzyl)piperazine-1-carboxylate (270 mg, yield: 68%) as a yellow solid. MS: m/z=571.2 [M+H]⁺.

### Step 3: 3-(5-Morpholino-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-morpholino-3H-imidazo[4,5-b]pyridin-3-yl)benzyl)piperazine-1-carboxylate (270 mg, 473 μmol) in 1,4-dioxane (2 mL) was added 4 M HCl in 1,4-dioxane (2 mL) at 25 °C The mixture was stirred at 25 °C for 16 hr. The reaction was concentrated under reduced pressure to give 3-(5-morpholino-3-(4-(piperazin-1-ylmethyl)phenyl)-3H-imidazo[4,5-b]pyridin-2-yl)pyridin-2-amine (Intermediate 22, 240 mg, HCl salt, yield: 93%) as a yellow solid. MS: m/z=471.2 [M+H]⁺. ¹H NMR (400 MHz, Methanol-d₄) δ 8.13-7.99 (m, 2H), 7.96-7.82 (m, 3H), 7.66 (dd, J=8.4, 3.2 Hz, 2H), 7.14-7.02 (m, 1H), 7.01-6.90 (m, 1H), 4.66-4.59 (m, 2H), 3.82-3.77 (m, 4H), 3.73-3.69 (m, 6H), 3.68 (s, 2H), 3.60-3.55 (m, 4H).

### Intermediate 23: 3-(2-(2-Aminopyridin-3-yl)-3-(4-(piperazin-1-ylmethyl)phenyl)-3H-imidazo[4,5-b]pyridin-5-yl)benzonitrile

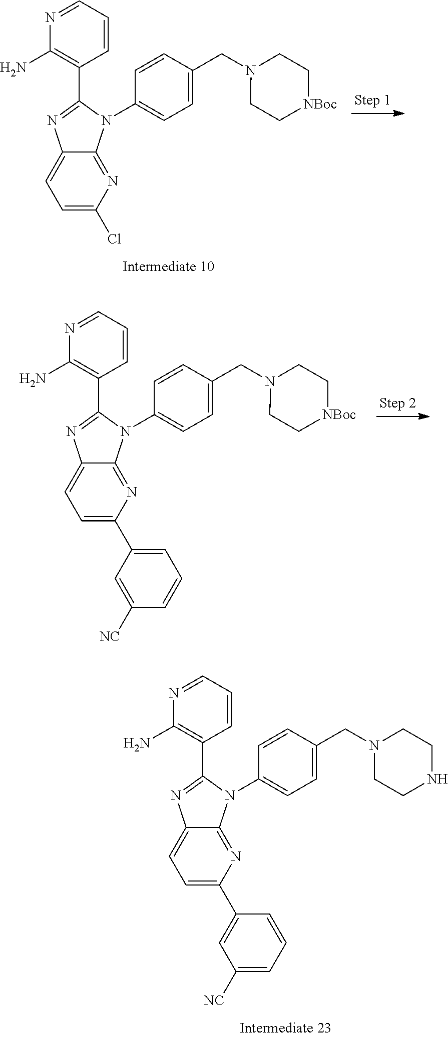 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(C#N)c5)nc43)cc2)CC1 ; N#Cc1cccc(-c2ccc3nc(-c4cccnc4N)n(-c4ccc(CN5CCNCC5)cc4)c3n2)c1]

### Step 1: tert-Butyl 4-(4-(2-(2-aminopyridin-3-yl)-5-(3-cyanophenyl)-3H-imidazo[4,5-b]pyridin-3-yl)benzyl)piperazine-1-carboxylate

A mixture of Intermediate 10 (220 mg, 423 μmol), (3-cyanophenyl)boronic acid (68.4 mg, 465 μmol), Cs₂CO₃ (414 mg, 1.27 mmol), Pd(dppf)Cl₂ (155 mg, 212 μmol) in 1,4-dioxane (5 mL) and H₂O (1 mL) was degassed and purged with N₂ three times. The mixture was stirred at 100 °C for 16 hr under N₂ atmosphere. The reaction mixture was filtered at 25 °C, diluted with H₂O (20 mL), and extracted with CH₂Cl₂ (20 mL×3). The combined organic layers were washed with brine (30 mL), dried over Na₂SO₄, filtered, and concentrated under reduced pressure. After purified by silica gel flash chromatography (Eluent of 0~30% EtOAc in petroleum ether), tert-butyl 4-(4-(2-(2-aminopyridin-3-yl)-5-(3-cyanophenyl)-3H-imidazo[4,5-b]pyridin-3-yl)benzyl)piperazine-1-carboxylate (240 mg, yield: 97%) was obtained as a brown solid. MS: m/z=587.4 [M+H]⁺.

### Step 2: 3-(2-(2-Aminopyridin-3-yl)-3-(4-(piperazin-1-ylmethyl)phenyl)-3H-imidazo[4,5-b]pyridin-5-yl)benzonitrile

To a solution of tert-butyl 4-(4-(2-(2-aminopyridin-3-yl)-5-(3-cyanophenyl)-3H-imidazo[4,5-b]pyridin-3-yl)benzyl)piperazine-1-carboxylate (240 mg, 409 μmol) in 1,4-dioxane (3 mL) was added 4 M HCl in 1,4-dioxane (3 mL) at 25 °C The mixture was stirred at 25 °C for 16 hr. The reaction mixture was filtered. The collected solid was washed with 1,4-dioxane (2 mL×2) and dried in vacuo to give 3-(2-(2-aminopyridin-3-yl)-3-(4-(piperazin-1-ylmethyl)phenyl)-3H-imidazo[4,5-b]pyridin-5-yl)benzonitrile (Intermediate 23, 203 mg HCl salt, yield: 95%) as a yellow solid. MS: m/z=487.2 [M+H]⁺. ¹H NMR (400 MHz, Methanol-d₄) δ 8.43-8.34 (m, 3H), 8.11 (d, J=8.4 Hz, 1H), 8.03 (d, J=5.6 Hz, 1H), 7.96 (d, J=8.4 Hz, 2H), 7.90 (d, J=7.6 Hz, 1H), 7.79-7.73 (m, 3H), 7.68-7.63 (m, 1H), 6.96-6.90 (m, 1H), 3.70-3.68 (m, 6H), 3.67-3.65 (m, 4H).

### Intermediate 24: 2-(2-(2-Aminopyridin-3-yl)-3-(4-(piperazin-1-ylmethyl)phenyl)-3H-imidazo[4,5-b]pyridin-5-yl)benzonitrile

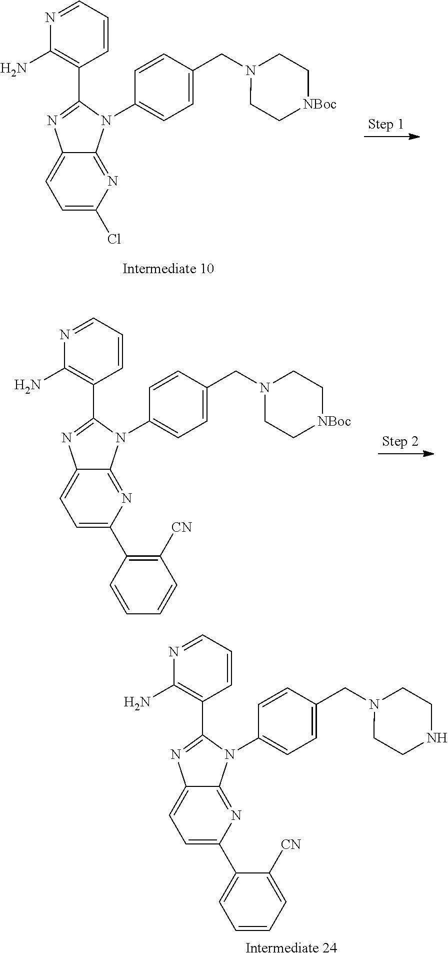

### Step 1: tert-Butyl 4-(4-(2-(2-aminopyridin-3-yl)-5-(2-cyanophenyl)-3H-imidazo[4,5-b]pyridin-3-yl)benzyl)piperazine-1-carboxylate

A mixture of Intermediate 10 (400 mg, 770 μmol), (2-cyanophenyl)boronic acid (124 mg, 846 μmol), K₃PO₄ (327 mg, 1.54 mmol), cataCXiumAPdG₃ (280 mg, 385 μmol), and tricyclohexylphosphane (22 mg, 76.9 μmol) in DMF (5 mL) was degassed and purged with N₂ three times. The mixture was stirred at 120 °C for 16 hr under N₂ atmosphere. The reaction mixture was filtered at 25 °C, diluted with H₂O (20 mL), and extracted with CH₂Cl₂ (20 mL×3). The combined organic layers were washed with brine (30 mL), dried over Na₂SO₄, filtered, and concentrated under reduced pressure. After purified by silica gel flash chromatography (Eluent of 20-80% EtOAc in Petroleum ether), tert-butyl 4-(4-(2-(2-aminopyridin-3-yl)-5-(2-cyanophenyl)-3H-imidazo[4,5-b]pyridin-3-yl)benzyl)piperazine-1-carboxylate (400 mg, yield: 80%) was obtained as a yellow solid. MS: m/z=587.2 [M+H]⁺.

### Step 2: 2-(2-(2-Aminopyridin-3-yl)-3-(4-(piperazin-1-ylmethyl)phenyl)-3H-imidazo[4,5-b]pyridin-5-yl)benzonitrile

To a solution of tert-butyl 4-(4-(2-(2-aminopyridin-3-yl)-5-(2-cyanophenyl)-3H-imidazo[4,5-b]pyridin-3-yl)benzyl)piperazine-1-carboxylate (400 mg, 682 μmol) in 1,4-dioxane (5 mL) was added 4 M HCl in 1,4-dioxane (3 mL) at 25 °C The mixture was stirred at 25 °C for 16 hr. The reaction mixture was filtered. The collected solid was washed with 1,4-dioxane (5 mL×2) and dried in vacuo to give 2-(2-(2-aminopyridin-3-yl)-3-(4-(piperazin-1-ylmethyl)phenyl)-3H-imidazo[4,5-b]pyridin-5-yl)benzonitrile (Intermediate 24, 268 mg HCl salt, yield: 75%) as a yellow solid. MS: m/z=487.2 [M+H]⁺. ¹H NMR (400 MHz, Dimethylsulfoxide-d₆) δ 8.38 (d, J=8.0 Hz, 1H), 8.03-7.99 (m, 1H), 7.97-7.92 (m, 1H), 7.90-7.77 (m, 3H), 7.64-7.58 (m, 1H), 7.50-7.37 (m, 5H), 7.19 (dd, J=7.6, 2.0 Hz, 1H), 6.98 (br s, 2H), 6.40 (dd, J=7.6, 4.8 Hz, 1H), 3.50 (s, 2H), 2.80-2.70 (m, 4H), 2.37-2.32 (m, 4H).

### Intermediate 25: 4-(2-(2-Aminopyridin-3-yl)-3-(4-(piperazin-1-ylmethyl)phenyl)-3H-imidazo[4,5-b]pyridin-5-yl)benzonitrile

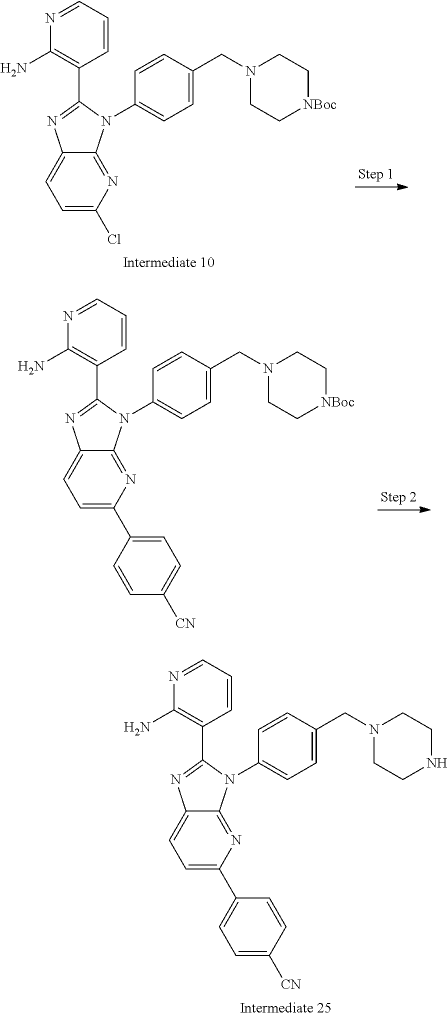

Following the general procedures described in Intermediate 23, the reactions were carried out using Intermediate 10. 4-(2-(2-Aminopyridin-3-yl)-3-(4-(piperazin-1-ylmethyl)phenyl)-3H-imidazo[4,5-b]pyridin-5-yl)benzonitrile (Intermediate 25, 192 mg HCl solid, yield: 98%) was obtained as a yellow solid. MS: m/z=487.2 [M+H]⁺. ¹H NMR (400 MHz, Methanol-d₄) δ 8.38 (d, J=8.4 Hz, 1H), 8.25 (d, J=8.4 Hz, 2H), 8.13 (d, J=8.4 Hz, 1H), 8.04 (d, J=5.6 Hz, 1H), 7.95 (d, J=7.6 Hz, 2H), 7.89 (d, J=7.2 Hz, 1H), 7.81 (d, J=8.4 Hz, 2H), 7.74 (d, J=7.6 Hz, 2H), 6.93 (t, J=6.8 Hz, 1H), 3.71-3.70 (m, 6H), 3.68-3.64 (m, 4H).

### Intermediate 26: 3-(5-(3-Fluorophenyl)-3-(4-(piperazin-1-ylmethyl)phenyl)-3H-imidazo[4,5-h]pyridin-2-yl)pyridin-2-amine

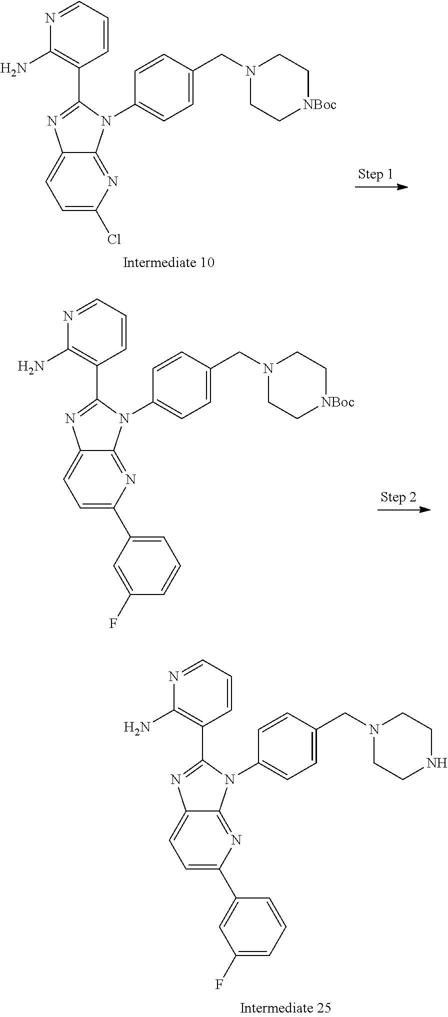 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(F)c5)nc43)cc2)CC1 ; Nc1ncccc1-c1nc2ccc(-c3cccc(F)c3)nc2n1-c1ccc(CN2CCNCC2)cc1]

Following the general procedures described in Intermediate 23, the reactions were carried out using Intermediate 10. 3-(5-(3-Fluorophenyl)-3-(4-(piperazin-1-ylmethyl)phenyl)-3H-imidazo[4,5-b]pyridin-2-yl)pyridin-2-amine (Intermediate 26, 174 mg HCl salt, yield: 98%) was obtained as a yellow solid. MS: m/z=480.1 [M+H]⁺. ¹H NMR (400 MHz, Methanol-d₄) δ 8.34 (d, J=8.4 Hz, 1H), 8.08-8.01 (m, 2H), 7.98-7.86 (m, 4H), 7.81-7.71 (m, 3H), 7.52-7.42 (m, 1H), 7.18-7.12 (m, 1H), 6.98-6.85 (m, 1H), 3.69-3.67 (m, 4H), 3.35 (s, 2H), 3.34-3.31 (m, 4H). ¹⁹F NMR (400 MHz, Methanol-d₄) δ −115.02.

### Intermediate 27: 3-(5-(3-Chlorophenyl)-3-(4-(piperazin-1-ylmethyl)phenyl)-3H-imidazo[4,5-b]pyridin-2-yl)pyridin-2-amine

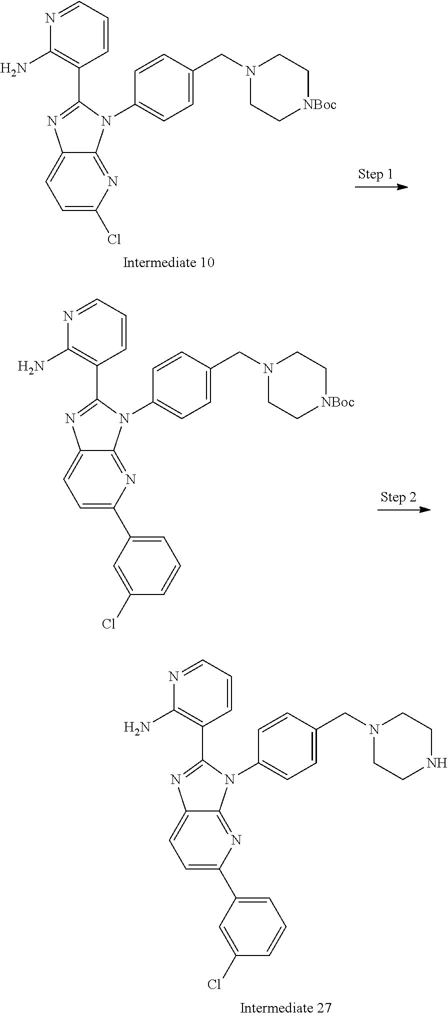 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(Cl)c5)nc43)cc2)CC1 ; Nc1ncccc1-c1nc2ccc(-c3cccc(Cl)c3)nc2n1-c1ccc(CN2CCNCC2)cc1]

Following the general procedures described in Intermediate 23, the reactions were carried out using Intermediate 10. 3-(5-(3-Chlorophenyl)-3-(4-(piperazin-1-ylmethyl)phenyl)-3H-imidazo[4,5-b]pyridin-2-yl)pyridin-2-amine (Intermediate 27, 113 mg, HCl salt, yield: 84%) was obtained as a yellow solid. MS: m/z=496.1 [M+H]⁺. ¹H NMR (400 MHz, Methanol-d₄) δ 8.21 (d, J=8.4 Hz, 1H), 8.04 (s, 1H), 8.01-7.92 (m, 3H), 7.55 (d, J=8.0 Hz, 2H), 7.45 (d, J=8.4 Hz, 2H), 7.43-7.33 (m, 3H), 6.48 (dd, J=7.6, 5.2 Hz, 1H), 3.64 (s, 2H), 2.92 (t, J=5.2 Hz, 4H), 2.50-2.58 (m, 4H).

### Intermediate 28: 5-(2-(2-Aminopyridin-3-yl)-3-(4-(piperazin-1-ylmethyl)phenyl)-3H-imidazo[4,5-b]pyridin-5-yl)-1-methylpyridin-2(1H)-one

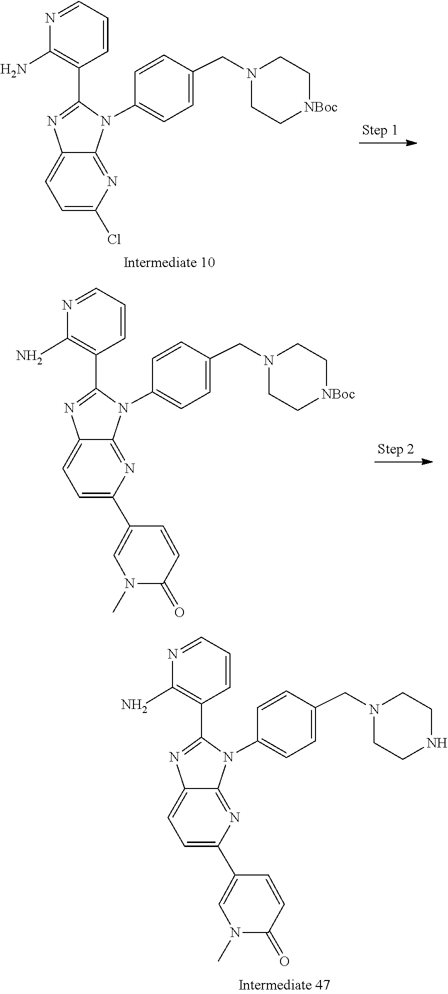

Following the general procedures described in Intermediate 23, the reactions were carried out using Intermediate 10. 5-(2-(2-Aminopyridin-3-yl)-3-(4-(piperazin-1-ylmethyl)phenyl)-3H-imidazo[4,5-b]pyridin-5-yl)-1-methylpyridin-2(1H)-one (Intermediate 28, 10.4 mg HCl salt, yield: 47%) was obtained as a yellow solid. MS: m/z=493.3 [M+H]⁺. ¹H NMR (400 MHz, Methanol-d₄) δ 8.49 (d, J=1.6 Hz, 1H), 8.34-8.26 (m, 2H), 8.02 (d, J=6.0 Hz, 1H), 7.96-7.90 (m, 3H), 7.86 (d, J=7.6 Hz, 1H), 7.72 (d, J=8.4 Hz, 2H), 6.91 (t, J=6.8 Hz, 1H), 6.66 (d, J=9.2 Hz, 1H), 3.74-3.66 (m, 10H), 3.60 (s, 3H).

### Intermediate 29: 6-(2-(2-Aminopyridin-3-yl)-3-(4-(piperazin-1-ylmethyl)phenyl)-3H-imidazo[4,5-b]pyridin-5-yl)pyridin-2(1H)-one

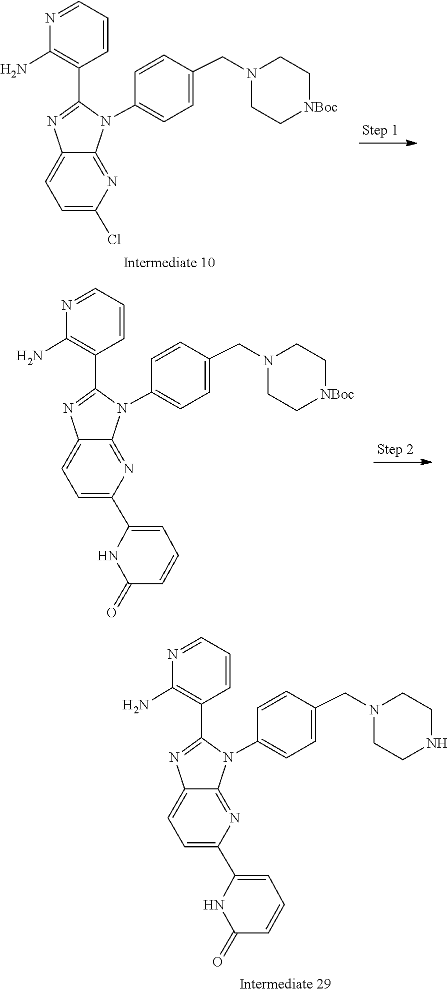 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(=O)[nH]5)nc43)cc2)CC1 ; Nc1ncccc1-c1nc2ccc(-c3cccc(=O)[nH]3)nc2n1-c1ccc(CN2CCNCC2)cc1]

Following the general procedures described in Intermediate 23, the reactions were carried out using Intermediate 10. 6-(2-(2-Aminopyridin-3-yl)-3-(4-(piperazin-1-ylmethyl)phenyl)-3H-imidazo[4,5-b]pyridin-5-yl)pyridin-2(1H)-one (Intermediate 29, 68.2 mg, yield: 52%) as a yellow solid. MS: m/z=479.2 [M+H]⁺. ¹H NMR (400 MHz, Dimethylsulfoxide-d₆) δ 8.33 (d, J=8.4 Hz, 1H), 8.16 (d, J=8.4 Hz, 1H), 8.01 (dd, J=4.8, 2.0 Hz, 1H), 7.60 (dd, J=8.8, 6.8 Hz, 1H), 7.52-7.41 (m, 5H), 7.20-7.14 (m, 2H), 7.02 (br s, 2H), 6.46 (d, J=8.8 Hz, 1H), 6.38 (dd, J=7.6, 4.8 Hz, 1H), 3.53 (s, 2H), 2.73-2.69 (m, 4H), 2.36-2.30 (m, 4H).

### Intermediate 30: 2-Methoxy-4-(piperidin-4-ylamino)benzaldehyde

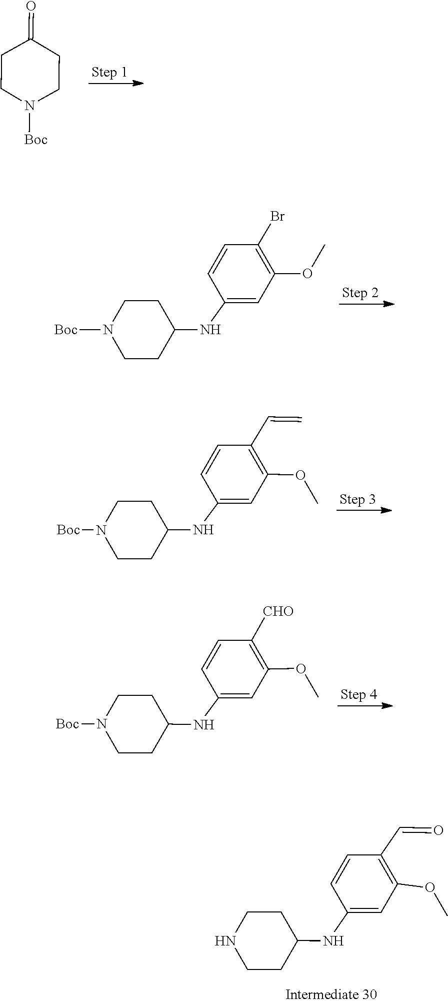

### Step 1: tert-Butyl 4-((4-bromo-3-methoxyphenyl)amino)piperidine-1-carboxylate

To a solution of tert-butyl 4-oxopiperidine-1-carboxylate (1 g, 4.95 mmol) and 4-bromo-3-methoxyaniline (1.08 g, 5.44 mmol) in CH₂Cl₂ (10 mL) was added acetic acid (446 mg, 7.42 mmol) at 0 °C After stirring at 0 °C for 0.5 hr, NaBH(OAc)₃ (1.57 g, 7.42 mmol) was added to the mixture. The resulting mixture was stirred at 25 °C for 16 hr. The reaction mixture was alkalized with 1M NaOH to adjust pH to around 9 and extracted with CH₂Cl₂ (20 mL×2). The combined organic layers were washed with brine (20 mL×2), dried over Na₂SO₄, filtered and concentrated under reduced pressure. tert-Butyl 4-((4-bromo-3-methoxyphenyl)amino)piperidine-1-carboxylate (1.9 g, yield: 86%) was obtained as a white solid. MS: m/z=385.0, 387.0 [M+H]⁺.

### Step 2: tert-Butyl 4-((3-methoxy-4-vinylphenyl)amino)piperidine-1-carboxylate

To a solution of tert-butyl 4-((4-bromo-3-methoxyphenyl)amino)piperidine-1-carboxylate (500 mg, 1.3 mmol) and Pd(dppf)Cl₂ (95 mg, 823 μmol) in toluene (10 mL) was added tributyl(vinyl)stannane (2.03 g, 6.4 mmol). The mixture was degassed and purged with N₂ three times and stirred at 110 °C for 16 hr under N₂ atmosphere. The reaction mixture was quenched with aqueous KF (10 mL) at 25 °C and diluted with EtOAc (200 mL×2). The combined organic layers were washed with brine (200 mL×2), dried over Na₂SO₄, filtered, and concentrated under reduced pressure. After purified by silica gel flash chromatography (Eluent of 20%-50% EtOAc in petroleum ether), tert-butyl 4-((3-methoxy-4-vinylphenyl)amino)piperidine-1-carboxylate (160 mg, yield: 26%) was obtained as a yellow oil. MS: m/z=333.2 [M+H]⁺.

### Step 3: tert-Butyl 4-((4-formyl-3-methoxyphenyl)amino)piperidine-1-carboxylate

To a solution of tert-butyl 4-((3-methoxy-4-vinylphenyl)amino)piperidine-1-carboxylate (800 mg, 2.41 mmol) and K₂OsO₄ (26.6 mg, 72.2 μmol) in THF (9 mL) and H₂O (3 mL) was added NaIO₄ (1.54 g, 7.22 mmol). The mixture was stirred at 25 °C for 1 hr. The reaction mixture was diluted with H₂O (20 mL) and extracted with EtOAc (20 mL×2). The combined organic layers were washed with brine (20 mL×2), dried over Na₂SO₄, filtered, and concentrated under reduced pressure. After purified by silica gel flash chromatography (Eluent of 20%~35% EtOAc in petroleum ether), tert-butyl 4-((4-formyl-3-methoxyphenyl)amino)piperidine-1-carboxylate (200 mg, yield: 17%) was obtained as a yellow oil. MS: m/z=335.2 [M+H]⁺.

### Step 4: 2-Methoxy-4-(piperidin-4-ylamino)benzaldehyde

To a solution of tert-butyl 4-((4-formyl-3-methoxyphenyl)amino)piperidine-1-carboxylate (50 mg, 150 μmol) in 1,4-dioxane (2 mL) was added 4M HCl in 1,4-dioxane (2 mL). The mixture was stirred at 25 °C for 1 hr. The reaction mixture was concentrated under reduced pressure to give 2-methoxy-4-(piperidin-4-ylamino)benzaldehyde (Intermediate 30, 30 mg, HCl salt) was obtained as a yellow solid. MS: m/z=235.1 [M+Na]⁺.

### Intermediate 31: 2-Methoxy-5-(piperidin-4-ylamino)benzaldehyde

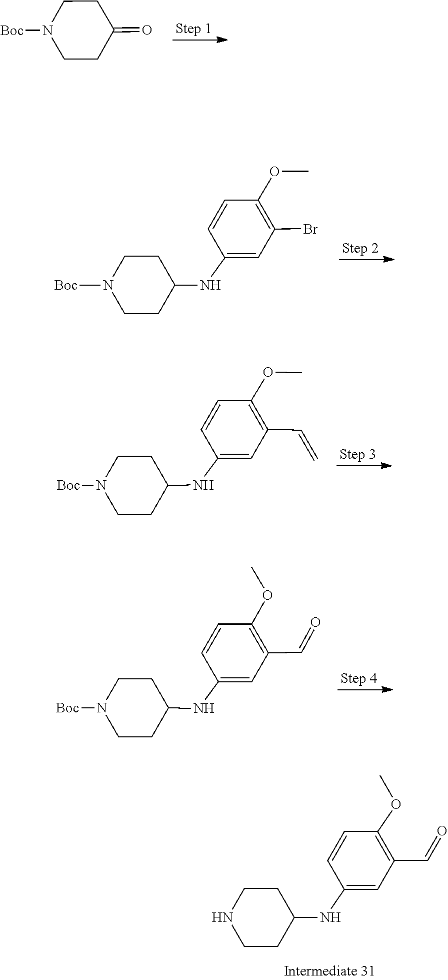 [SMILES: CC(C)(C)OC(=O)N1CCC(=O)CC1 ; COc1ccc(NC2CCN(C(=O)OC(C)(C)C)CC2)cc1Br ; C=Cc1cc(NC2CCN(C(=O)OC(C)(C)C)CC2)ccc1OC ; COc1ccc(NC2CCN(C(=O)OC(C)(C)C)CC2)cc1C=O ; COc1ccc(NC2CCNCC2)cc1C=O]

Following the general procedures described in Intermediate 30, the reactions were carried out using corresponding starting materials. 2-Methoxy-5-(piperidin-4-ylamino)benzaldehyde (Intermediate 31, 50 mg, HCl salt, yield: 59%) was obtained as a yellow solid. MS: m/z=235.1 [M+H]⁺.

### Intermediate 32: 3-Formyl-2-methoxy-N-(piperidin-4-yl)benzamide

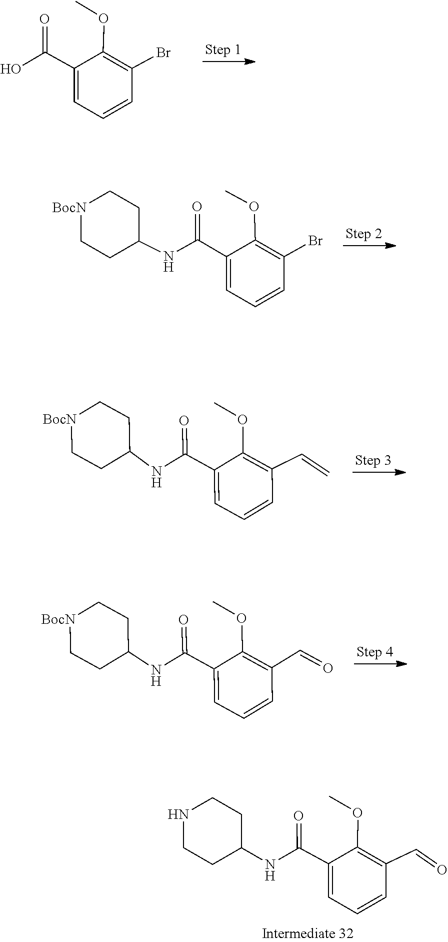

### Step 1: tert-Butyl 4-(3-bromo-2-methoxybenzamido)piperidine-1-carboxylate

To a solution of tert-butyl 4-aminopiperidine-1-carboxylate (2 g, 9.99 mmol) in DMF (10 mL) were added 3-bromo-2-methoxybenzoic acid (2.31 g, 9.99 mmol), HATU (5.70 g, 14.9 mmol) and DIEA (3.87 g, 29.9 mmol). The mixture was stirred at 25 °C for 16 hr. The mixture was diluted with H₂O (200 mL), extracted with EtOAc (200 mL) and washed with H₂O (200 mL×2). The combined organic layers were washed with brine (200 mL×2), dried over Na₂SO₄, filtered and concentrated under reduced pressure to give tert-butyl 4-(3-bromo-2-methoxybenzamido)piperidine-1-carboxylate (4 g, yield: 85%) as a gray solid. MS: m/z=358.8 [M-55]⁺. ¹H NMR (400 MHz, Dimethylsulfoxide-d₆) δ 8.30 (d, J=8.0 Hz, 1H), 7.71 (d, J=8.0 Hz, 1H), 7.42 (d, J=7.2 Hz, 1H), 7.15-7.10 (m, 1H), 3.93-3.85 (m, 2H), 3.77 (s, 3H), 3.71-3.54 (m, 1H), 3.24-3.07 (m, 1H), 2.91-2.88 (m, 1H), 1.84-1.75 (m, 2H), 1.40 (s, 9H), 1.38-1.31 (m, 2H)

### Step 2: tert-Butyl 4-(2-methoxy-3-vinylbenzamido)piperidine-1-carboxylate

To a solution of tert-butyl 4-(3-bromo-2-methoxybenzamido)piperidine-1-carboxylate (2 g, 4.84 mmol) in 1,4-dioxane (20 mL) and H₂O (2 mL) were added 4,4,5,5-tetramethyl-2-vinyl-1,3,2-dioxaborolane (894 mg, 5.81 mmol), Cs₂CO₃ (3.15 g, 9.68 mmol) and Pd(dppf)Cl₂ (354 mg, 484 μmol). The mixture was degassed and purged with N₂ three times. The mixture was stirred at 100 °C for 16 hr under N₂ atmosphere. The mixture was diluted with H₂O (200 mL) and extracted with CH₂Cl₂ (200 mL×2). The combined organic layers were washed with brine (200 mL×2), dried over Na₂SO₄, filtered and concentrated under reduced pressure. After purified by silica gel flash chromatography (Eluent of 0~15% EtOAc in petroleum ether), tert-butyl 4-(2-methoxy-3-vinylbenzamido)piperidine-1-carboxylate (1.36 g, yield: 75%) was obtained as a yellow oil. MS: m/z=305.1 [M+H-55]⁺. ¹H NMR (400 MHz, Dimethylsulfoxide-d₆) 58.20 (d, J=8.0 Hz, 1H), 7.68 (dd, J=7.6, 1.6 Hz, 1H), 7.38 (dd, J=7.2, 1.6 Hz, 1H), 7.20-7.14 (m, 1H), 6.96 (dd, J=18.0, 11.2 Hz, 1H), 5.88 (dd, J=18.0, 1.2 Hz, 1H), 5.44 (dd, J=10.8, 0.8 Hz, 1H), 3.94-3.92 (m, 1H), 3.91-3.83 (m, 2H), 3.70 (s, 3H), 2.99-2.80 (m, 2H), 1.84-1.76 (m, 2H), 1.40 (s, 9H), 1.39-1.31 (m, 2H).

### Step 3: tert-Butyl 4-(3-formyl-2-methoxybenzamido)piperidine-1-carboxylate

To a solution of tert-butyl 4-(2-methoxy-3-vinylbenzamido)piperidine-1-carboxylate (1.2 g, 3.33 mmol) in THF (20 mL) and H₂O (5 mL) were added K₂OsO₄·2H₂O (123 mg, 333 μmol) and NaIO₄ (2.14 g, 9.99 mmol) at 0 °C The mixture was stirred at 25 °C for 2 hr. The mixture was diluted with H₂O (100 mL) and extracted with EtOAc (100 mL×2). The combined organic layers were washed with brine (100 mL×2), dried over Na₂SO₄, filtered and concentrated under reduced pressure to give tert-butyl 4-(3-formyl-2-methoxybenzamido)piperidine-1-carboxylate (1.1 g, yield: 74%) as a black brown solid. MS: m/z=307.1 [M-55]⁺.

### Step 4: 3-Formyl-2-methoxy-N-(piperidin-4-yl)benzamide

To a solution of tert-butyl 4-(3-formyl-2-methoxybenzamido)piperidine-1-carboxylate (1.1 g, 3.04 mmol) in CH₂Cl₂ (5 mL) was added 4 M HCl in 1,4-dioxane (759 μL). The mixture was stirred at 25 °C for 4 hr. The mixture was filtered and the filter cake was concentrated under reduced pressure to give 3-formyl-2-methoxy-N-(piperidin-4-yl)benzamide (Intermediate 32, 600 mg, yield: 67%) as a yellow solid. MS: m/z=263.1 [M+H]⁺.

### Intermediate 33: 2-Methoxy-3-(piperidin-4-ylamino)benzaldehyde

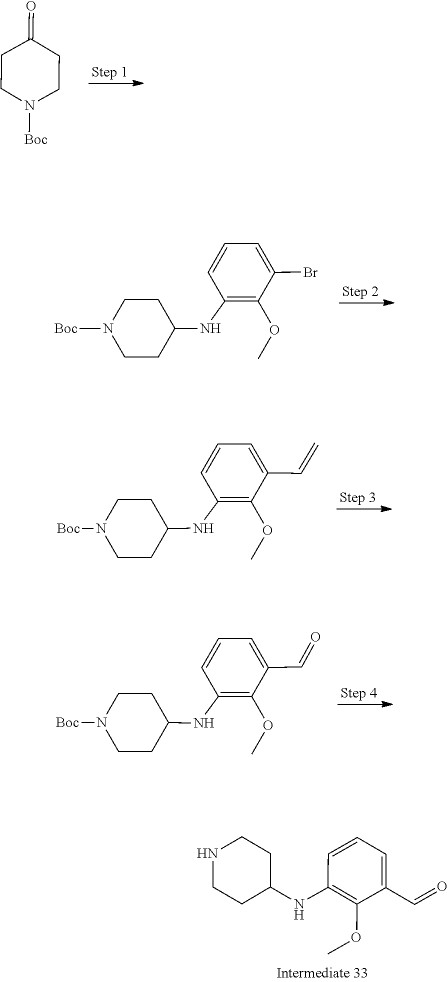 [SMILES: CC(C)(C)OC(=O)N1CCC(=O)CC1 ; COc1c(Br)cccc1NC1CCN(C(=O)OC(C)(C)C)CC1 ; C=Cc1cccc(NC2CCN(C(=O)OC(C)(C)C)CC2)c1OC ; COc1c(C=O)cccc1NC1CCN(C(=O)OC(C)(C)C)CC1 ; COc1c(C=O)cccc1NC1CCNCC1]

Following the general procedures described in Intermediate 30, the reactions were carried out using corresponding starting materials. 2-Methoxy-3-(piperidin-4-ylamino)benzaldehyde (Intermediate 33, 150 mg, yield: 71%) was obtained as a yellow solid. MS: m/z=235.2 [M+H]⁺.

### Intermediate 34: 2-Methoxy-6-(piperidin-4-ylamino)benzaldehyde

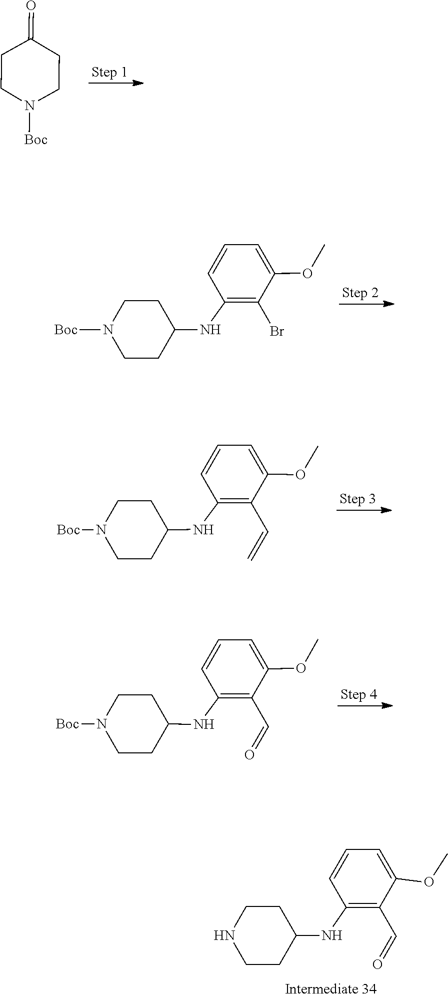

Following the general procedures described in Intermediate 30, the reactions were carried out using corresponding starting materials. 2-Methoxy-6-(piperidin-4-ylamino)benzaldehyde (Intermediate 34, 121 mg, yield: 100%, HCl) was obtained as a yellow solid. MS: m/z=234.9 [M+H]⁺. ¹H NMR (400 MHz, Methanol-d₄) δ 10.33 (s, 1H), 7.39 (t, J=8.4 Hz, 1H), 6.46 (d, J=8.8 Hz, 1H), 6.26 (d, J=8.4 Hz, 1H), 3.90-3.80 (m, 4H), 3.51-3.38 (m, 2H), 3.25-3.10 (m, 2H), 2.34-2.23 (m, 2H), 1.88-1.61 (in, 2H).

### Intermediate 35: 3-(3-(4-((4-Aminopiperidin-1l-yl)methyl)phenyl)-5-morpholino-3H-imidazo[4,5-b]pyridin-2-yl)pyridin-2-amine

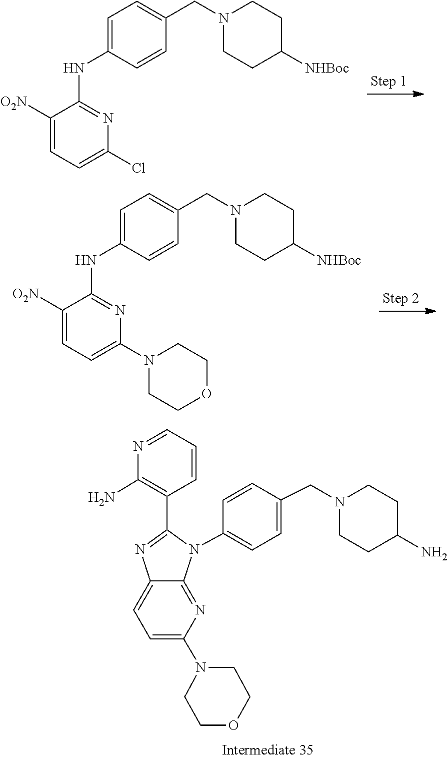

### Step 1: tert-Butyl (1-(4-((6-morpholino-3-nitropyridin-2-yl)amino)benzyl)piperidin-4-yl)carbamate

A mixture of tert-butyl (1-(4-((6-chloro-3-nitropyridin-2-yl)amino)benzyl)piperidin-4-yl)carbamate (refer to Intermediate 4 for detail procedures, 3 g, 6.49 mmol), morpholine (678 mg, 7.79 mmol), DIEA (1.68 g, 12.9 mmol) in MeCN (40 mL) was stirred at 90 °C for 2 hr. The reaction mixture was diluted with H₂O (100 mL) and extracted with CH₂Cl₂ (100 mL×3). The combined organic layers were washed with H₂O (50 mL×2), dried over Na₂SO₄, filtered and concentrated to give tert-butyl (1-(4-((6-morpholino-3-nitropyridin-2-yl)amino)benzyl)piperidin-4-yl)carbamate (3.24 g, yield: 97%) as a yellow solid, which was directly used in the next step. MS: m/z=513.2 [M+H]⁺. ¹H NMR (400 MHz, Chloroform-d) 10.70 (s, 1H), 8.32 (d, J=9.2 Hz, 1H), 7.52 (d, J=8.4 Hz, 2H), 7.30 (d, J=8.0 Hz, 2H), 6.12 (d, J=9.6 Hz, 1H), 4.53-4.34 (m, 1H), 3.79-3.75 (m, 4H), 3.75-3.55 (m, 4H), 3.48 (s, 3H), 2.90-2.75 (m, 2H), 2.15-2.05 (m, 2H), 1.95-1.75 (m, 2H), 1.76-1.53 (m, 2H), 1.44 (s, 9H).

### Step 2: 3-(3-(4-((4-Aminopiperidin-1-yl)methyl)phenyl)-5-morpholino-3H-imidazo[4,5-b]pyridin-2-yl)pyridin-2-amine

A mixture of tert-butyl (1-(4-((6-morpholino-3-nitropyridin-2-yl)amino)benzyl)piperidin-4-yl)carbamate (3.24 g, 6.32 mmol), 2-aminopyridine-3-carbaldehyde (926 mg, 7.58 mmol), and Na₂S₂O₄ (3.88 g, 18.9 mmol) in DMSO (90 mL) was stirred at 100 °C for 24 hr. The reaction mixture was diluted with H₂O (200 mL) and extracted with CH₂Cl₂ (50 mL×3). The pH of aqueous phase was adjusted to about 8-9 with sat. Na₂CO₃ (100 mL). The mixture was extracted with EtOAc (50 mL×6), dried over Na₂SO₄, filtered and concentrated under reduced pressure. After purified by silica gel flash chromatography (Eluent of 0-7% MeOH in CH₂Cl₂), 3-(3-(4-((4-aminopiperidin-1-yl)methyl)phenyl)-5-morpholino-3H-imidazo[4,5-b]pyridin-2-yl)pyridin-2-amine (Intermediate 35, 1.2 g, yield: 29%) was obtained as a yellow solid. MS: m/z=485.3 [M+H]⁺. ¹H NMR (400 MHz, Dimethylsulfoxide-d₆) δ 7.98 (d, J=8.8 Hz, 1H), 7.96 (dd, J=4.8, 2.0 Hz, 1H), 7.40 (d, J=8.4 Hz, 2H), 7.29 (d, J=8.4 Hz, 2H), 7.03 (br s, 2H), 6.98 (dd, J=8.0, 1.6 Hz, 1H), 6.89 (d, J=8.8 Hz, 1H), 6.30 (dd, J=8.0, 4.8 Hz, 1H), 3.69-3.65 (m, 4H), 3.50 (s, 2H), 3.41-3.38 (m, 5H), 2.78-2.70 (m, 2H), 2.02-1.92 (m, 2H), 1.70-1.64 (m, 2H), 1.35-1.16 (m, 2H).

### Intermediate 36: 3-(3-(4-(Chloromethyl)phenyl)-6-phenyl-3H-imidazo[4,5-b]pyridin-2-yl)pyridin-2-amine

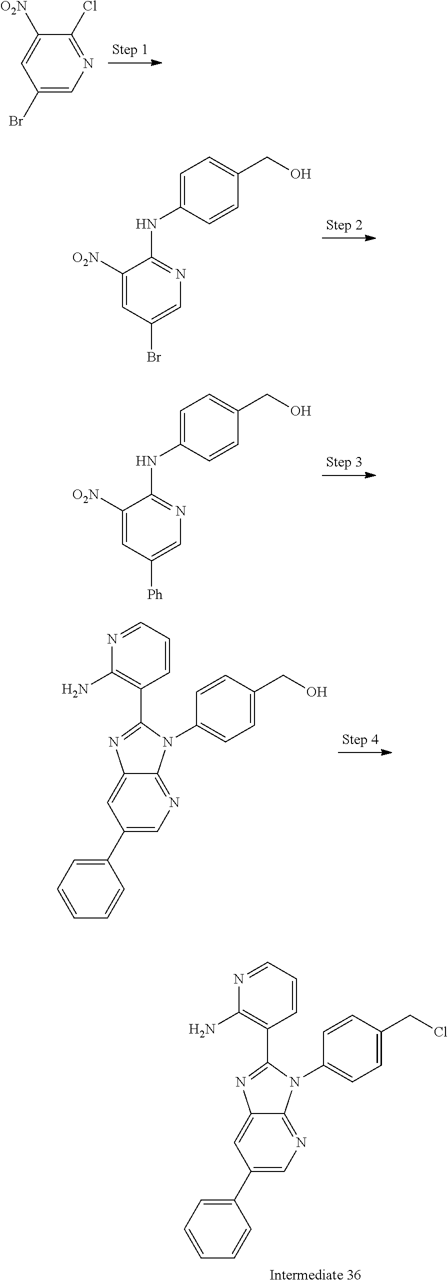

### Step 1: (4-((5-Bromo-3-nitropyridin-2-yl)amino)phenyl)methanol

To a solution of 5-bromo-2-chloro-3-nitro-pyridine (10 g, 42 mmol) and (4-aminophenyl)methanol (5.2 g, 42 mmol) in DMSO (100 mL) was added DIEA (16.3 g, 126 mmol). The mixture was stirred at 25 °C for 16 hr. The reaction mixture was poured into H₂O (200 mL) and extracted with EtOAc (300 mL×2). The combined organic layers were washed with brine (200 mL×2), dried over anhydrous Na₂SO₄, filtered, and concentrated to dryness. The residue was purified by silica gel flash chromatography (Eluent of 10-50% EtOAc in petroleum ether) to give (4-((5-bromo-3-nitropyridin-2-yl)amino)phenyl)methanol (6.28 g, yield: 46%) as a red solid. MS: m/z=324.0, 325.0 [M+H]⁺. ¹H NMR (400 MHz, Chloroform-d-) δ 10.05 (br s, 1H), 8.65 (d, J=2.4 Hz, 1H), 8.50 (d, J=2.4 Hz, 1H), 7.59 (d, J=8.4 Hz, 2H), 7.40 (d, J=8.4 Hz, 2H), 4.71 (s, 2H).

### Step 2: (4-((3-Nitro-5-phenylpyridin-2-yl)amino)phenyl)methanol

A mixture of [4-[(5-bromo-3-nitro-2-pyridyl)amino]phenyl]methanol (5.4 g, 16.7 mmol), phenylboronic acid (6.1 g, 50 mmol), K₂CO₃ (4.62 g, 33.44 mmol), Pd(dppf)Cl₂ (1.22 g, 1.67 mmol) in 1,4-dioxane (60 mL) and H₂O (12 mL) was degassed and purged with N₂ three times. The mixture was stirred at 100 °C for 8 hr under N₂ atmosphere. The reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated under reduced pressure. The residue was purified by silica gel flash chromatography (Eluent of 0~50% EtOAc in petroleum ether) to give [4-[(3-nitro-5-phenyl-2-pyridyl)amino]phenyl]methanol (4.6 g, yield: 86%) as a red solid. MS: m/z=322.1 [M+H]^{t}. ¹H NMR (400 MHz, Chloroform-d) δ 10.15 (s, 1H), 8.78-8.72 (m, 2H), 8.25 (d, J=8.0 Hz 1H), 7.70-7.65 (m, 2H), 7.59-7.56 (m, 2H), 7.51-7.49 (m, 2H), 7.44-7.41 (m, 2H), 4.72 (s, 2H).

### Step 3: (4-(2-(2-Aminopyridin-3-yl)-6-phenyl-3H-imidazo[4,5-b]pyridin-3-yl)phenyl)methanol

To a solution of [4-[(3-nitro-5-phenyl-2-pyridyl)amino]phenyl]methanol (15 g, 46.7 mmol) in DMSO (600 mL) were added Na₂S₂O₄ (16 g, 93 mmol) and 2-aminopyridine-3-carbaldehyde (6.8 g, 56 mmol). The mixture was stirred at 100 °C for 12 hr. After cooling to 25 °C, the reaction mixture was diluted with H₂O (200 mL) and extracted with CH₂Cl₂ (200 ml×3). The combined organic layers were washed with brine (200 ml×2), dried over Na₂SO₄, filtered, and concentrated under reduced pressure. The residue was purified by silica gel flash chromatography (Eluent of 0~100% EtOAc in petroleum ether) to give [4-[2-(2-amino-3-pyridyl)-6-phenyl-imidazo[4,5-b]pyridin-3-yl]phenyl]methanol (5.15 g crude, yield: 28%) as a yellow solid. MS: m/z=394.0 [M+H]⁺.

### Step 4: 3-(3-(4-(Chloromethyl)phenyl)-6-phenyl-3H-imidazo[4,5-b]pyridin-2-yl)pyridin-2-amine

To a solution of [4-[2-(2-amino-3-pyridyl)-6-phenyl-imidazo[4,5-b]pyridin-3-yl]phenyl]methanol (500 mg, 1.3 mmol) in CH₂Cl₂ (10 mL) was added SOCl₂ (771 mg, 6.5 mmol). The mixture was stirred at 40 °C for 2 hr. The reaction mixture was concentrated under reduced pressure. The residue was purified by silica gel flash chromatography (Eluent of 0-100% EtOAc in petroleum ether) to give 3-(3-(4-(chloromethyl)phenyl)-6-phenyl-3H-imidazo[4,5-b]pyridin-2-yl)pyridin-2-amine (Intermediate 36, 267 mg, yield: 51%) as a yellow solid. MS: m/z=411.8 [M+H]⁺. ¹H NMR (400 MHz, Dimethylsulfoxide-di) δ 8.63 (d, J=2.0 Hz, 1H), 8.47 (d, J=2.0 Hz, 1H), 8.01 (dd, J=8.8, 3.2 Hz, 1H), 7.79 (d, J=7.2 Hz, 2H), 7.61 (d, J=8.4 Hz, 2H), 7.55-7.47 (m, 4H), 7.45-7.40 (m, 1H), 7.25 (dd, J=7.6, 1.8 Hz, 1H), 6.96 (br s, 2H), 6.44 (dd, 1=7.6, 4.8 Hz, 1H), 4.86 (s, 2H).

### Intermediate 37: 3-(3-(4-((4-(Methylamino)piperidin-1-yl)methyl)phenyl)-5-phenyl-3H-imidazo[4,5-b]pyridin-2-yl)pyridin-2-amine

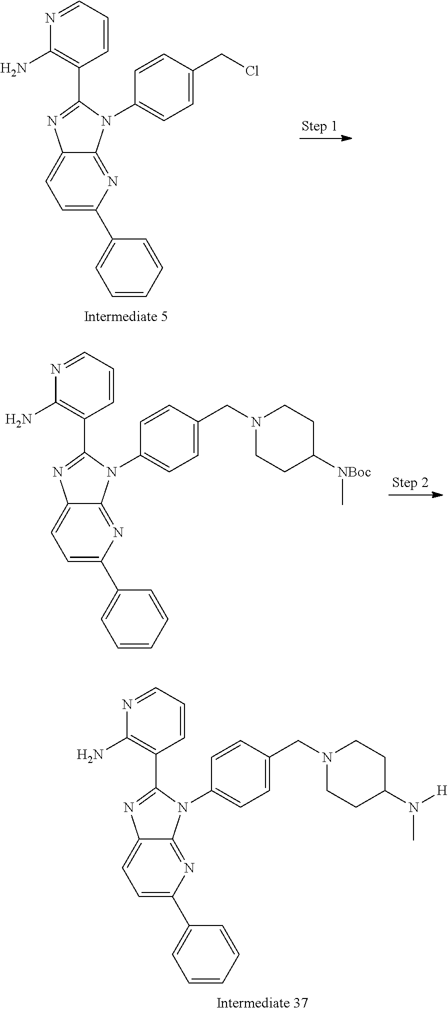

### Step 1: 3-[3-[4-[[4-(methylamino)-1-piperidyl]methyl]phenyl]-5-phenyl-imidazo[4,5-b]pyridin-2-yl]pyridin-2-amine

To a solution of Intermediate 5 (300 mg, 0.73 mmol) and tert-butyl N-methyl-N-(4-piperidyl)carbamate (312 mg, 1.46 mmol) in DMF (2 mL) were added NaI (18.2 mg, 0.073 mmol) and K₂CO₃ (336 mg, 2.43 mmol). The mixture was stirred at 80 °C for 2 hr. After cooling to 20 °C, the reaction mixture was poured into H₂O (3 mL) and extracted with EtOAc (10 mL×3). The combined organic layers were dried over anhydrous Na₂SO₄, filtered, and concentrated under reduced pressure. The residue was purified by silica gel flash chromatography (Eluent of 0~7% MeOH in CH₂Cl₂) to give tert-butyl N-[l-[[4-[2-(2-amino-3-pyridyl)-5-phenyl-imidazo[4,5-b]pyridin-3-yl]phenyl]methyl]-4-piperidyl]-N-methyl-carbamate (220 mg, yield: 51%,) as a yellow solid. MS: m/z=590.3 [M+H]⁺.

### Step 2: 3-(3-(4-((4-(Methylamino)piperidin-1-yl)methyl)phenyl)-5-phenyl-3H-imidazo[4,5-b]pyridin-2-yl)pyridin-2-amine

To a solution of tert-butyl N-[1-[[4-[2-(2-amino-3-pyridyl)-5-phenyl-imidazo[4,5-b]pyridin-3-yl]phenyl]methyl]-4-piperidyl]-N-methyl-carbamate (200 mg, 0.339 mmol) in CH₂Cl₂ (2 mL) was added TFA (38.7 mg, 0.339 mmol). The mixture was stirred at 25 °C for 2 hr. The reaction mixture was concentrated to give a residue (Intermediate 37, 185 mg TFA salt, yield: 93%). The crude (50 mg) was diluted with aqueous NaHCO₃ (10 mL), extracted with CH₂Cl₂ (10 mL×3), and purified by prep-TLC (CH₂Cl₂:MeOH=10:1) to give 3-(3-(4-((4-(methylamino)piperidin-1-yl)methyl)phenyl)-5-phenyl-3H-imidazo[4,5-b]pyridin-2-yl)pyridin-2-amine (Intermediate 37) as an off-white solid. MS: m/z=490.2 [M+H]⁺. ¹H NMR (400 MHz, Dimethylsulfoxide-d₆) δ 8.27 (d, J=8.4 Hz, 1H), 8.07-7.94 (m, 4H), 7.53-7.43 (m, 6H), 7.41-7.39 (m, 1H), 7.16 (dd, J=7.8, 1.2 Hz, 1H), 7.02 (br s, 2H), 6.37 (dd, J=7.4, 4.8 Hz, 1H), 3.55 (s, 2H), 3.47-3.37 (m, 1H), 2.80 (br d, J=12.0 Hz, 2H), 2.31 (s, 3H), 2.01 (dd, J=11.4, 9.8 Hz, 2H), 1.85-1.77 (m, 2H), 1.36-1.22 (m, 2H).

### Intermediate 38: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

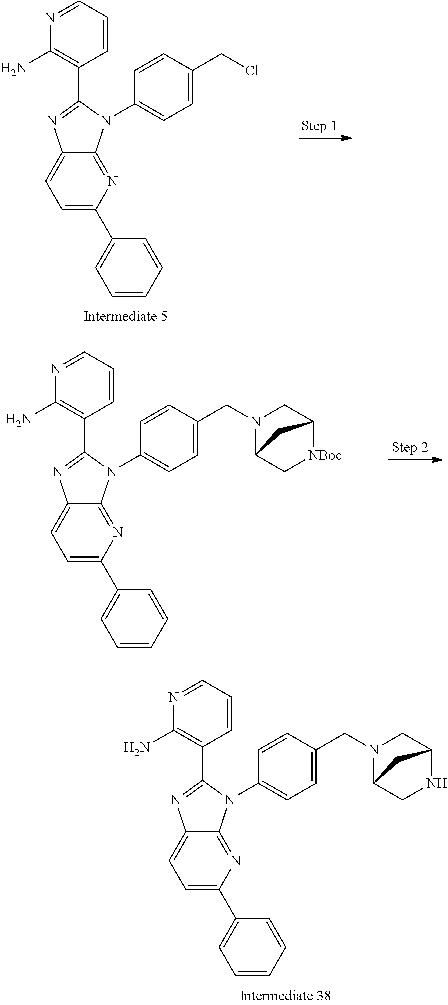 [SMILES: Nc1ncccc1-c1nc2ccc(-c3ccccc3)nc2n1-c1ccc(CCl)cc1 ; CC(C)(C)OC(=O)N1C[C@H]2C[C@@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@H]3C[C@@H]2CN3)cc1]

Following the general procedures described in Intermediate 37, the reactions were carried out using Intermediate 5. 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine (Intermediate 38, 854 mg, 2HCl salt, yield: 90%) was obtained as a yellow solid. MS: m/z=474.2 [M+H]⁺. ¹H NMR (400 MHz, Methanol-d₄) δ 8.33 (d, J=8.4 Hz, 1H), 8.06-8.00 (m, 6H), 7.88 (dd, J=7.6, 1.6 Hz, 1H), 7.73 (d, J=8.4 Hz, 2H), 7.46-7.40 (m, 3H), 6.92 (dd, J=7.6, 6.4 Hz, 1H), 4.84-4.81 (m, 1H), 4.75-4.65 (m, 2H), 4.14 (d, J=12.8 Hz, 1H), 3.96-3.92 (m, 1H), 3.74-3.71 (m, 1H), 3.62-3.60 (m, 1H), 3.38-3.32 (m, 1H), 2.87-2.82 (m, 1H), 2.39-2.36 (m, 1H).

### Intermediate 39: 2-Hydroxy-5-(methyl(piperidin-4-yl)amino)benzaldehyde

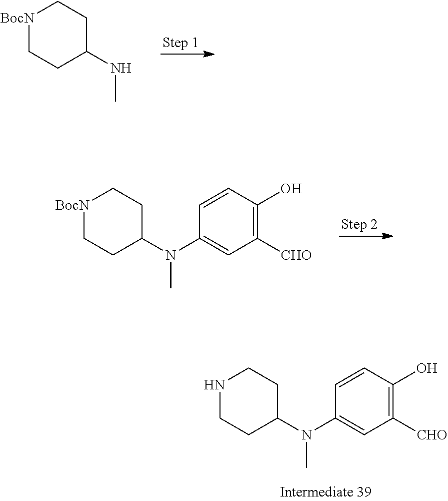 [SMILES: CNC1CCN(C(=O)OC(C)(C)C)CC1 ; CN(c1ccc(O)c(C=O)c1)C1CCN(C(=O)OC(C)(C)C)CC1 ; CN(c1ccc(O)c(C=O)c1)C1CCNCC1]

### Step 1: tert-Butyl 4-((3-formyl-4-hydroxyphenyl)(methyl)amino)piperidine-1-carboxylate

To a solution of tert-butyl 4-(methylamino)piperidine-1-carboxylate (306 mg, 1.43 mmol) and 4-fluoro-2-hydroxybenzaldehyde (200 mg, 1.43 mmol) in DMSO (2 mL) was added DIEA (553 mg, 4.28 mmol). The mixture was stirred at 100 °C for 16 hr. The reaction mixture was diluted with H₂O (10 mL) and extracted with EtOAc (30 mL). The combined organic layers were washed with brine (20 mL), dried over Na₂SO₄, filtered, and concentrated under reduced pressure. After purified by silica gel flash chromatography (Eluent of 0%-16% EtOAc in petroleum ether), tert-butyl 4-((3-formyl-4-hydroxyphenyl)(methyl)amino)piperidine-1-carboxylate (126 mg, yield: 26%) was obtained as an off white solid. MS: m/z=335.1 [M+H]⁺.

### Step 2: 2-Hydroxy-5-(methyl(piperidin-4-yl)amino)benzaldehyde

To a solution of tert-butyl 4-((3-formyl-4-hydroxyphenyl)(methyl)amino)piperidine-1-carboxylate (150 mg, 451 μmol) in CH₂Cl₂ (2 mL) was added 4M HCl in 1,4-dioxane (1.68 mL). The mixture was stirred at 25 °C for 1 hr. The reaction mixture was concentrated under reduced pressure to give 2-hydroxy-5-(methyl(piperidin-4-yl)amino)benzaldehyde (Intermediate 39, 102 mg, HCl salt) as an off white solid, which was used in the next step without further purification.

### Intermediate 40: (R)-3-(3-(4-((3-Aminopiperidin-1-yl)methyl)phenyl)-5-phenyl-3H-imidazo[4,5-b]pyridin-2-yl)pyridin-2-amine

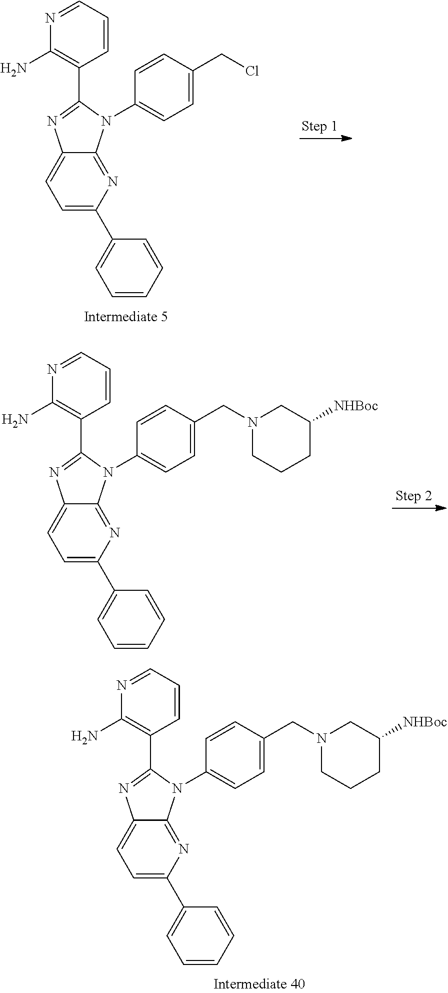

Following the general procedures described in Intermediate 37, the reactions were carried out using corresponding starting materials. After purified by prep-TLC (CH₂Cl₂:MeOH=10:1), (R)-3-(3-(4-((3-aminopiperidin-1-yl)methyl)phenyl)-5-phenyl-3H-imidazo[4,5-b]pyridin-2-yl)pyridin-2-amine (Intermediate 40, 6.0 mg, yield: 11.5%) was obtained as a light-yellow powder. MS: m/z=476.3 [M+H]⁺. ¹H NMR (400 MHz, Dimethylsulfoxide-d₆) δ 8.27 (d, J=8.0 Hz, 1H), 8.04-7.97 (m, 4H), 7.50-7.44 (m, 6H), 7.42-7.38 (m, 1H), 7.16 (dd, J=7.6, 1.6 Hz, 1H), 7.01 (br s, 2H), 6.39 (dd, J=7.6, 4.8 Hz, 1H), 3.62-3.52 (m, 2H), 2.90-2.85 (m, 1H), 2.79-2.74 (m, 1H), 2.62-2.59 (m, 1H), 2.09-2.00 (m, 1H), 1.95-1.87 (m, 1H), 1.80-1.74 (m, 1H), 1.70-1.64 (m, 1H), 1.53-1.45 (m, 1H), 1.26-1.21 (m, 1H).

### Intermediate 41: (R)-3-(3-(4-((3-Aminopyrrolidin-1-yl)methyl)phenyl)-5-phenyl-3H-imidazo[4,5-b]pyridin-2-yl)pyridin-2-amine

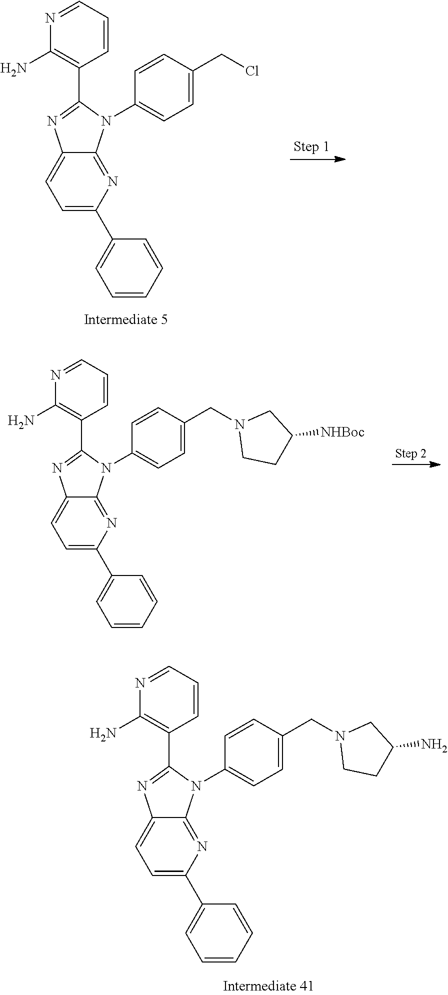 [SMILES: Nc1ncccc1-c1nc2ccc(-c3ccccc3)nc2n1-c1ccc(CCl)cc1 ; CC(C)(C)OC(=O)N[C@@H]1CCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CC[C@@H](N)C2)cc1]

Following the general procedures described in Intermediate 37, the reactions were carried out using Intermediate 5. After purified by prep-TLC (CH₂Cl₂:MeOH=10:1), (R)-3-(3-(4-((3-aminopyrrolidin-1-yl)methyl)phenyl)-5-phenyl-3H-imidazo[4,5-b]pyridin-2-yl)pyridin-2-amine (Intermediate 41, 7.7 mg) was obtained as a light-yellow powder. MS: m/z=462.3 [M+H]⁺. ¹H NMR (400 MHz, Dimethylsulfoxide-d₆) δ 8.27 (d, J=8.0 Hz, 1H), 8.04-7.97 (m, 4H), 7.52-7.44 (m, 6H), 7.43-7.34 (m, 1H), 7.6 (dd, J=7.6, 2.0 Hz, 1H), 7.00 (br s, 2H), 6.40 (dd, J=8.0, 5.2 Hz, 1H), 3.69 (s, 2H), 3.60-3.57 (m, 2H), 2.75-2.66 (m, 2H), 2.46-2.40 (m, 1H), 2.18-2.08 (m, 1H), 1.66-1.56 (m, 1H).

### Intermediate 42: 3-(3-(4-((3-Aminoazetidin-1-yl)methyl)phenyl)-5-phenyl-3H-imidazo[4,5-b]pyridin-2-yl)pyridin-2-amine

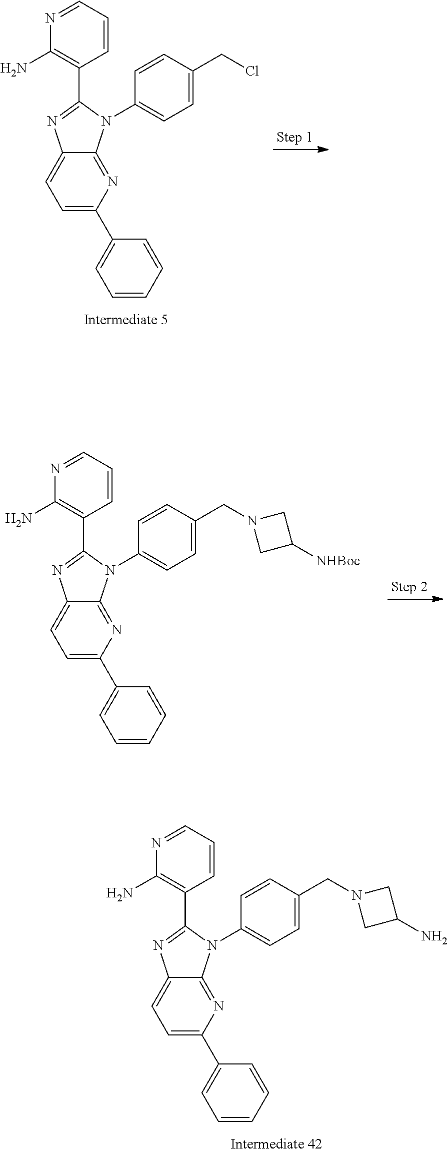 [SMILES: Nc1ncccc1-c1nc2ccc(-c3ccccc3)nc2n1-c1ccc(CCl)cc1 ; CC(C)(C)OC(=O)NC1CN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CC(N)C2)cc1]

Following the general procedures described in Intermediate 37, the reactions were carried out using Intermediate 5. 3-(3-(4-((3-Aminoazetidin-1-yl)methyl)phenyl)-5-phenyl-3H-imidazo[4,5-b]pyridin-2-yl)pyridin-2-amine (Intermediate 42, 31 mg, TFA salt, yield: 53%) was obtained, which was used in the next step without further purification. MS: m/z=448.2 [M+H]⁺. ¹H NMR (400 MHz, Dimethylsulfoxide-d₆) δ 8.26 (d, J=8.4 Hz, 1H), 8.04-7.96 (m, 4H), 7.49-7.36 (m, 7H), 7.15 (dd, J=7.6, 1.6 Hz, 1H), 7.00 (br s, 2H), 6.39 (dd, J=8.0, 4.8 Hz, 1H), 3.61 (s, 2H), 3.52-3.48 (m, 2H), 3.47-3.38 (m, 3H), 2.72-2.65 (m, 2H).

### Intermediate 43: 3-(3-(4-((2,6-Diazaspiro[3.3]heptan-2-yl)methyl)phenyl)-5-phenyl-3H-imidazo[4,5-b]pyridin-2-yl)pyridin-2-amine

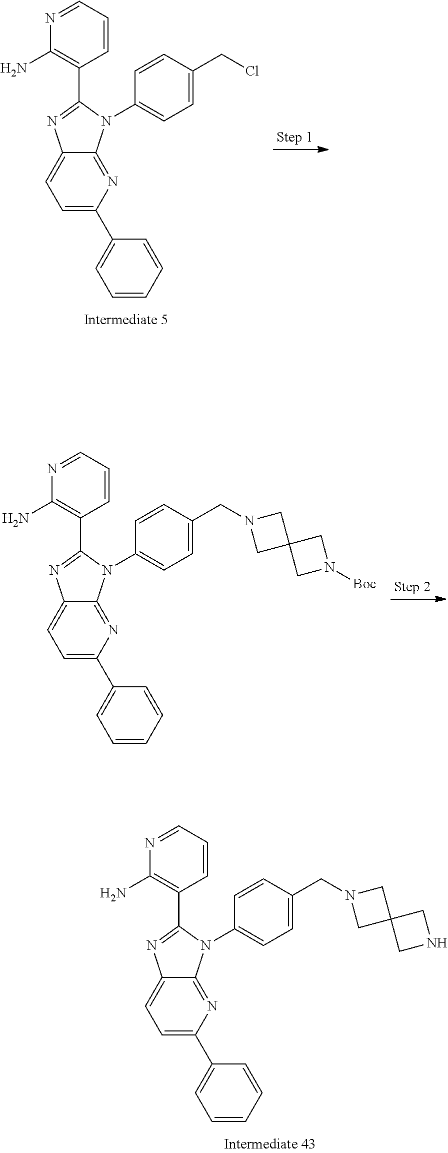 [SMILES: Nc1ncccc1-c1nc2ccc(-c3ccccc3)nc2n1-c1ccc(CCl)cc1 ; CC(C)(C)OC(=O)N1CC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CNC3)C2)cc1]

Following the general procedures described in Intermediate 37, the reactions were carried out using Intermediate 5. 3-(3-(4-((2,6-Diazaspiro[3.3]heptan-2-yl)methyl)phenyl)-5-phenyl-3H-imidazo[4,5-b]pyridin-2-yl)pyridin-2-amine (Intermediate 43, 120 mg, TFA salt, yield: 81%) was obtained as a yellow solid. MS: m/z=474.2 [M+H]⁺. ¹H NMR (400 MHz, Dimethylsulfoxide-d₆) δ 8.26 (d, J=8.4 Hz, 1H), 8.04-7.97 (m, 4H), 7.49-7.44 (m, 2H), 7.42-7.37 (m, 5H), 7.15 (dd, J=7.6, 1.6 Hz, 1H), 6.99 (br s, 2H), 6.39 (dd, J=8.0, 5.2 Hz, 1H), 3.58 (s, 2H), 3.56 (s, 4H), 3.25 (s, 4H), 1.23 (s, 1H).

### Intermediate 44: 3-(3-(4-((2,7-Diazaspiro[3.5]nonan-7-yl)methyl)phenyl)-5-phenyl-3H-imidazo[4,5-b]pyridin-2-yl)pyridin-2-amine

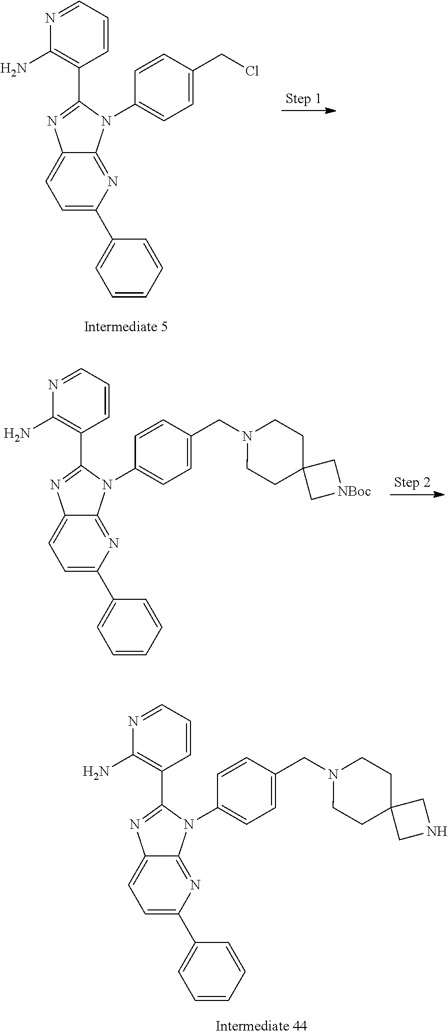 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CNC3)cc1]

Following the general procedures described in Intermediate 37, the reactions were carried out using Intermediate 5. After purified by prep-HPLC (column: Waters xbridge 150*25 mm 10 μm; mobile phase: [water (NH₄HCO₃)-ACN]; B %: 32%-62%, 8 min), 3-(3-(4-((2,7-Diazaspiro[3.5]nonan-7-yl)methyl)phenyl)-5-phenyl-3H-imidazo[4,5-b]pyridin-2-yl)pyridin-2-amine (Intermediate 44, 300 mg, yield: 74%) was obtained as a light-yellow oil. MS: m/z=502.3 [M+H]⁺. ¹H NMR (400 MHz, Dimethylsulfoxide-d₆) δ 8.26 (d, J=8.4 Hz, 1H), 8.05-7.97 (m, 4H), 7.49-7.39 (m, 7H), 7.18-7.12 (dd, J=7.6, 1.6 Hz, 1H), 7.02 (br s, 2H), 6.36 (dd, J=7.6, 5.2 Hz, 1H), 3.58-3.41 (m, 6H), 2.38-2.26 (m, 4H), 1.71-1.68 (m, 4H).

### Intermediate 45: 7-(4-(2-(2-Aminopyridin-3-yl)-5-phenyl-3H-imidazo[4,5-b]pyridin-3-yl)benzyl)-7-azaspiro[3.5]nonan-2-amine

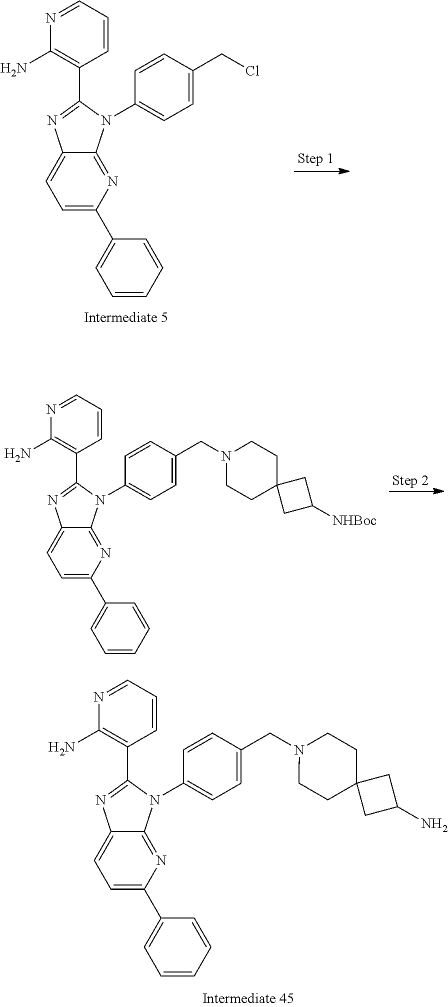

Following the general procedures described in Intermediate 37, the reactions were carried out using Intermediate 5. After purified by prep-HPLC (column: Phenomenex C18 150*25 mm 10 μm; mobile phase: [water (NH₄HCO₃)-ACN]; B %: 28%-58%, 8 min), 7-(4-(2-(2-Aminopyridin-3-yl)-5-phenyl-3H-imidazo[4,5-b]pyridin-3-yl)benzyl)-7-azaspiro[3.5]nonan-2-amine (Intermediate 45, 370 mg, yield: 91%) as a light-yellow oil. MS: m/z=516.3 [M+H]⁺. ¹H NMR (400 MHz, Dimethylsulfoxide-d₆) δ 8.27 (d, J=8.0 Hz, 1H), 8.06-7.95 (m, 4H), 7.55-7.35 (m, 7H), 7.18-7.12 (m, 1H), 7.01 (br s, 2H), 6.36 (dd, 1=7.6, 4.8 Hz, 1H), 3.71-3.58 (m, 1H), 3.52 (s, 2H), 3.01-2.98 (m, 4H), 2.41-2.20 (m, 2H), 2.14-2.05 (m, 2H), 1.90-1.80 (m, 2H), 1.67-1.53 (m, 2H).

### Intermediate 46: 2-(4-(2-(2-Aminopyridin-3-yl)-5-phenyl-3H-imidazo[4,5-b]pyridin-3-yl)benzyl)-2-azaspiro[3.3]heptan-6-amine

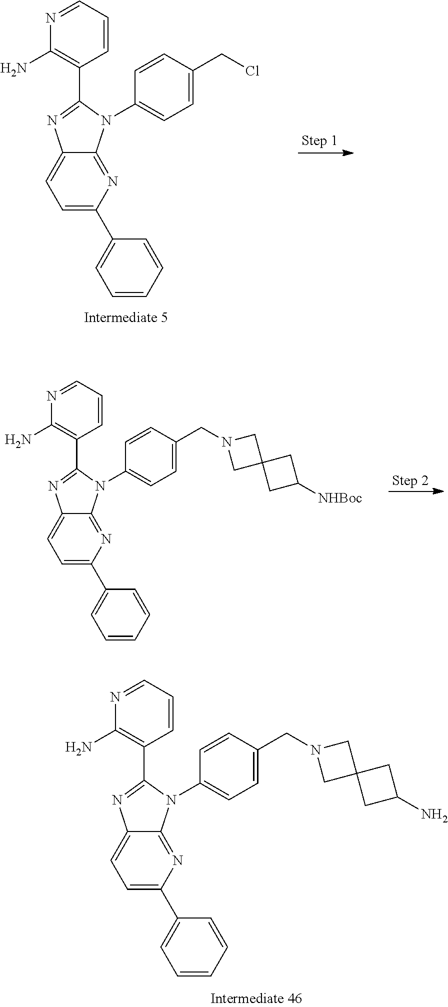

Following the general procedures described in Intermediate 37, the reactions were carried out using corresponding starting materials. After purified by prep-TLC (CH₂Cl₂:MeOH=10:1), 2-(4-(2-(2-aminopyridin-3-yl)-5-phenyl-3H-imidazo[4,5-b]pyridin-3-yl)benzyl)-2-azaspiro[3.3]heptan-6-amine (Intermediate 46, 123 mg, yield: 83%) was obtained as a light-yellow solid. MS: m/z=488.3 [M+H]⁺. ¹H NMR (400 MHz, Methanol-d₄) δ 8.20 (d, J=8.8 Hz, 1H), 8.05-8.01 (m, 2H), 7.99 (dd, J=5.2, 1.6 Hz, 1H), 7.95 (d, J=8.4 Hz, 1H), 7.58-7.50 (m, 2H), 7.43-7.39 (m, 2H), 7.34 (dd, J=7.6, 2.0 Hz, 1H), 6.49 (dd, J=7.6, 4.8 Hz, 1H), 6.21-6.17 (m, 2H), 5.65 (dd, J=7.6, 4.4 Hz, 1H), 4.26-4.18 (m, 1H), 4.00 (s, 2H), 3.77 (s, 2H), 3.66 (s, 2H), 2.65-2.60 (m, 2H), 2.22-2.17 (m, 2H).

### Intermediate 47: 3-(3-(4-((1,4-Diazepan-1-yl)methyl)phenyl)-5-phenyl-3H-imidazo[4,5-b]pyridin-2-yl)pyridin-2-amine

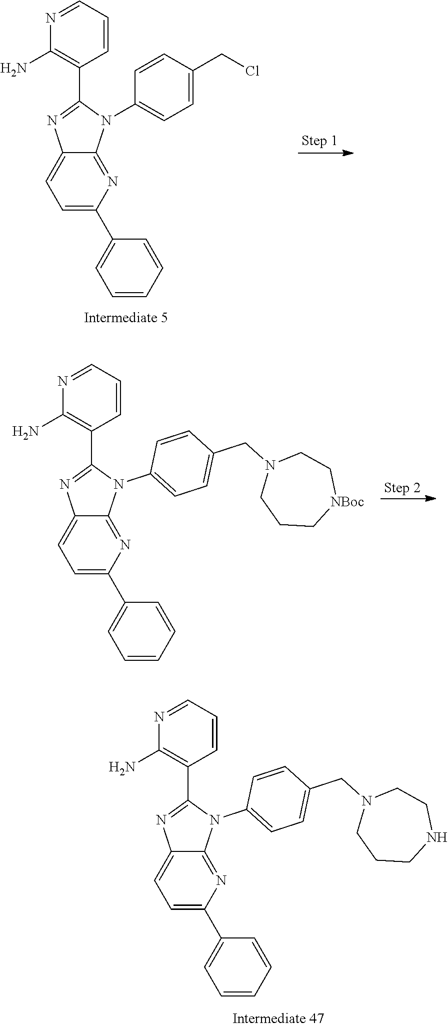 [SMILES: Nc1ncccc1-c1nc2ccc(-c3ccccc3)nc2n1-c1ccc(CCl)cc1 ; CC(C)(C)OC(=O)N1CCCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCCNCC2)cc1]

Following the general procedures described in Intermediate 37, the reactions were carried out using corresponding starting materials. 3-(3-(4-((1,4-Diazepan-1-yl)methyl)phenyl)-5-phenyl-3H-imidazo[4,5-b]pyridin-2-yl)pyridin-2-amine (Intermediate 47, 310 mg HCl salt, yield: 67%) was obtained and used in the next step without further purification. MS: m/z=476.3 [M+H]⁺. ¹H NMR (400 MHz, Dimethylsulfoxide-d₆) δ 8.4 (d, J=8.4 Hz, 1H), 8.04-7.97 (m, 4H), 7.52-7.39 (m, 7H), 7.14 (dd, J=7.6, 1.8 Hz, 1H), 7.05 (br s, 2H), 6.39-6.34 (m, 1H), 3.72 (s, 2H), 2.82 (t, J=6.4 Hz, 2H), 2.78-2.74 (m, 2H), 2.67 (t, J=6.4 Hz, 2H), 2.66-2.57 (m, 2H), 1.78-1.64 (m, 2H).

### Intermediate 48: 2-Hydroxy-4-(2,7-diazaspiro[3.5]nonan-2-yl)benzaldehyde

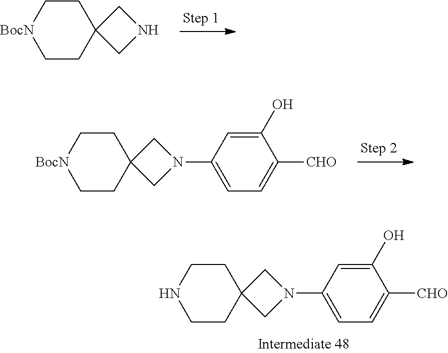 [SMILES: CC(C)(C)OC(=O)N1CCC2(CC1)CNC2 ; CC(C)(C)OC(=O)N1CCC2(CC1)CN(c1ccc(C=O)c(O)c1)C2 ; O=Cc1ccc(N2CC3(CCNCC3)C2)cc1O]

### Step 1: tert-Butyl 2-(4-formyl-3-hydroxyphenyl)-2,7-diazaspiro[3.5]nonane-7-carboxylate

To a solution of tert-butyl 2,7-diazaspiro[3.5]nonane-7-carboxylate (200 mg, 1.43 mmol) and 4-fluoro-2-hydroxybenzaldehyde (323 mg, 1.43 mmol) in DMSO (2 mL) was added DIEA (553 mg, 4.28 mmol). The resulting mixture was stirred at 100 °C for 16 hr. The reaction mixture was diluted with H₂O (20 mL) and extracted with EtOAc (20 mL×2). The combined organic layers were dried over Na₂SO₄, filtered, and concentrated under reduced pressure. After purified by silica gel flash chromatography (Eluent of 0~23% EtOAc in petroleum ether), tert-butyl 2-(4-formyl-3-hydroxyphenyl)-2,7-diazaspiro[3.5]nonane-7-carboxylate (200 mg, yield: 39%) was obtained as a white solid. MS: m/z=347.1 [M+H]⁺. ¹H NMR (400 MHz, Chloroform-d) δ 11.68 (s, 1H), 9.51 (s, 1H), 7.28 (s, 1H), 5.97 (dd, J=8.8, 2.0 Hz, 1H), 5.78 (d, J=2.0 Hz, 1H), 3.73 (s, 4H), 3.42-3.39 (m, 4H), 1.80-1.76 (m, 4H), 1.46 (s, 9H).

### Step 2: 2-Hydroxy-4-(2,7-diazaspiro[3.5]nonan-2-yl)benzaldehyde

To a solution of tert-butyl 2-(4-formyl-3-hydroxyphenyl)-2,7-diazaspiro[3.5]nonane-7-carboxylate (200 mg, 577 μmol) in 1,4-dioxane (2 mL) was added HCl in 1,4-dioxane (4M, 2 mL). The mixture was stirred at 25 °C for 1 hr. The reaction mixture was concentrated under reduced pressure to give 2-hydroxy-4-(2,7-diazaspiro[3.5]nonan-2-yl)benzaldehyde (Intermediate 48, 140 mg, HCl salt, yield: 85%) as a brown solid. MS: m/z=247.0 [M+H]⁺.

### Intermediate 49: 3-(3-(4-((2,8-Diazaspiro[4.5]decan-8-yl)methyl)phenyl)-5-phenyl-3H-imidazo[4,5-b]pyridin-2-yl)pyridin-2-amine

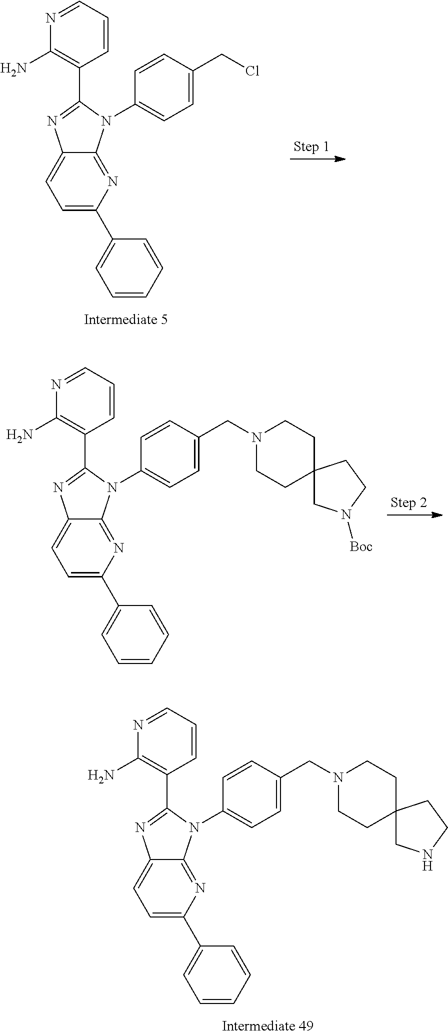 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCNC3)CC2)cc1]

Following the general procedures described in Intermediate 37, the reactions were carried out using corresponding starting materials. 3-(3-(4-((2,8-Diazaspiro[4.5]decan-8-yl)methyl)phenyl)-5-phenyl-3H-imidazo[4,5-b]pyridin-2-yl)pyridin-2-amine (Intermediate 49, 200 mg, yield: 85%) was obtained. MS: m/z=516.3 [M+H]⁺. ¹H NMR (400 MHz, Methanol-d₄) δ 8.18 (d, J=8.4 Hz, 1H), 8.02 (d, J=7.2 Hz, 2H), 7.97 (dd, J=4.8, 1.6 Hz, 1H), 7.93 (d, J=8.4 Hz, 1H), 7.52 (d, J=8.4 Hz, 2H), 7.45-7.34 (m, 5H), 7.31 (dd, J=6.8, 1.6 Hz, 1H), 6.45 (dd, J=7.6, 4.8 Hz, 1H), 3.62 (s, 2H), 3.10 (t, J=7.2 Hz, 2H), 2.85 (s, 2H), 2.50 (m, 4H), 1.75 (t, J=7.2 Hz, 2H), 1.65 (t, J=5.6 Hz, 4H).

### Intermediate 50: 2-Hydroxy-4-(2,8-diazaspiro[4.5]decan-2-yl)benzaldehyde

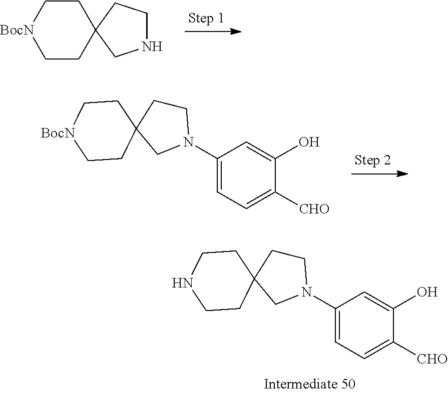

Following the general procedures described in Intermediate 48, the reactions were carried out using corresponding starting materials. 2-Hydroxy-4-(2,8-diazaspiro[4.5]decan-2-yl)benzaldehyde (Intermediate 50, 180 mg, HCl salt) was obtained as an off white solid.

### Intermediate 51: 3-(3-(4-((3,9-diazaspiro[5.5]undecan-3-yl)methyl)phenyl)-5-phenyl-3H-imidazo[4,5-b]pyridin-2-yl)pyridin-2-amine

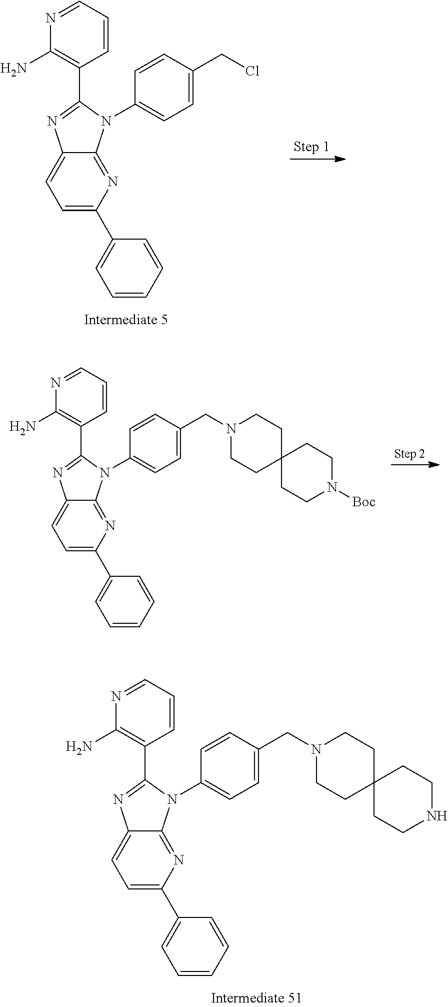 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)CC1 ; Nc1ncccc1-c1nc2ccc(-c3ccccc3)nc2n1-c1ccc(CN2CCC3(CCNCC3)CC2)cc1]

Following the general procedures described in Intermediate 37, the reactions were carried out using corresponding starting materials. 3-(3-(4-((3,9-Diazaspiro[5.5]undecan-3-yl)methyl)phenyl)-5-phenyl-3H-imidazo[4,5-b]pyridin-2-yl)pyridin-2-amine (Intermediate 51, 121 mg, HCl salt, yield: 67%) was obtained as a yellow solid. MS: m/z=530.3 [M+H]⁺. ¹H NMR (400 MHz, Dimethylsulfoxide-d₆) δ 10.81-10.50 (m, 1H), 8.79-8.66 (m, 2H), 8.38 (dd, J=8.4 Hz, 1H), 8.13-8.11 (m, 1H), 8.08-8.05 (m, 4H), 7.86-7.77 (m, 3H), 7.67 (d, J=8.0 Hz, 2H), 7.51-7.46 (m, 2H), 7.45-7.41 (m, 1H), 6.86-6.78 (m, 1H), 4.41 (d, J=4.4 Hz, 2H), 3.21-3.15 (m, 2H), 3.09-3.01 (m, 6H), 1.91-1.85 (m, 2H), 1.82-1.71 (m, 4H), 1.54-1.53 (m, 2H).

### Intermediate 52: 2-Hydroxy-4-(3,9-diazaspiro[5.5]undecan-3-yl)benzaldehyde

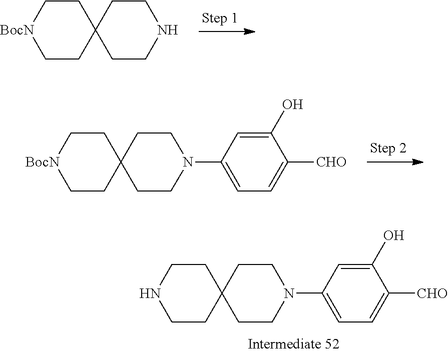 [SMILES: CC(C)(C)OC(=O)N1CCC2(CCNCC2)CC1 ; CC(C)(C)OC(=O)N1CCC2(CC1)CCN(c1ccc(C=O)c(O)c1)CC2 ; O=Cc1ccc(N2CCC3(CCNCC3)CC2)cc1O]

Following the general procedures described in Intermediate 48, the reactions were carried out using corresponding starting materials. 2-Hydroxy-4-(3,9-diazaspiro[5.5]undecan-3-yl)benzaldehyde (Intermediate 52, 180 mg, HCl salt) was obtained as an off white solid. MS: m/z=275.1 [M+H]⁺.

### Intermediate 53: 2-Hydroxy-4-(2,6-diazaspiro[3.4]octan-2-yl)benzaldehyde

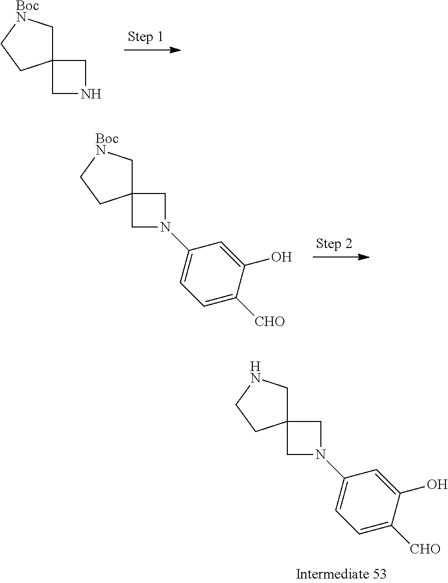 [SMILES: CC(C)(C)OC(=O)N1CCC2(CNC2)C1 ; CC(C)(C)OC(=O)N1CCC2(C1)CN(c1ccc(C=O)c(O)c1)C2 ; O=Cc1ccc(N2CC3(CCNC3)C2)cc1O]

Following the general procedures described in Intermediate 48, the reactions were carried out using corresponding starting materials. 2-Hydroxy-4-(2,6-diazaspiro[3.4]octan-2-yl)benzaldehyde (Intermediate 53, 300 mg, TFA salt, crude) was obtained as a brown oil. MS: m/z=233.0 [M+H]⁺.

### Intermediate 54: 3-(3-(4-((2,7-Diazaspiro[4.4]nonan-2-yl)methyl)phenyl)-5-phenyl-3H-imidazo[4,5-b]pyridin-2-1)pyridin-2-amine

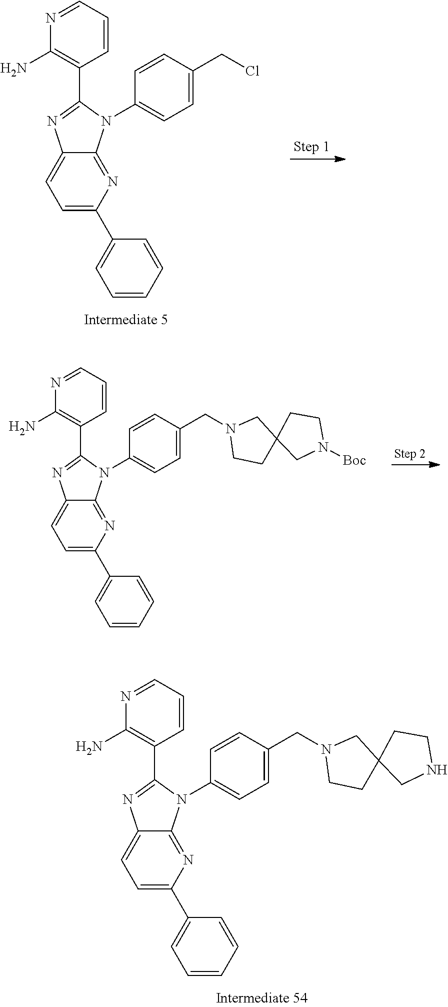 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCNC3)C2)cc1]

Following the general procedures described in Intermediate 37, the reactions were carried out using corresponding starting materials. 3-(3-(4-((2,7-Diazaspiro[4.4]nonan-2-yl)methyl)phenyl)-5-phenyl-3H-imidazo[4,5-b]pyridin-2-yl)pyridin-2-amine (Intermediate 54, 452 mg, HCl salt, yield: 86%) was obtained as a yellow solid. MS: m/z=502.3 [M+H]⁺. ¹H NMR (400 MHz, Dimethylsulfoxide-d₆) δ 11.93 (br s, 1H), 9.88-9.48 (m, 2H), 8.60-8.35 (m, 2H), 8.38 (d, J=8.4 Hz, 1H), 8.16 (dd, J=6.4, 1.6 Hz, 1H), 8.08-8.05 (m, 3H), 7.98-7.83 (m, 3H), 7.66 (d, J=8.4 Hz, 2H), 7.52-7.40 (m, 3H), 7.02-6.92 (m, 1H), 4.54-4.43 (m, 2H), 3.64-3.47 (m, 2H), 3.43-3.27 (m, 3H), 3.25-3.18 (m, 3H), 2.31-2.14 (m, 2H), 2.11-1.96 (m, 2H).

### Intermediate 55: 2-Hydroxy-4-(2,7-diazaspiro[4.4]nonan-2-yl)benzaldehyde

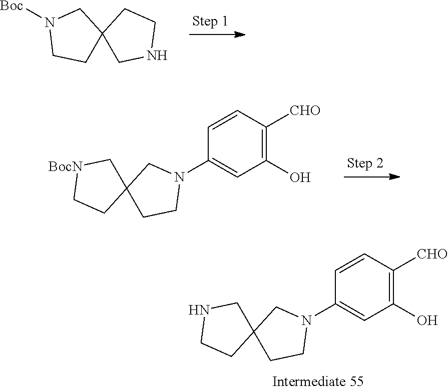 [SMILES: CC(C)(C)OC(=O)N1CCC2(CCNC2)C1 ; CC(C)(C)OC(=O)N1CCC2(CCN(c3ccc(C=O)c(O)c3)C2)C1 ; O=Cc1ccc(N2CCC3(CCNC3)C2)cc1O]

Following the general procedures described in Intermediate 48, the reactions were carried out using corresponding starting materials. 2-Hydroxy-4-(2,7-diazaspiro[4.4]nonan-2-yl)benzaldehyde (Intermediate 55, 40 mg, HCl salt, yield: 45%) was obtained as a yellow solid. MS: m/z=247.1 [M+H]⁺.

### Intermediate 56: 2-Hydroxy-5-(2,7-diazaspiro[4.4]nonan-2-yl)benzaldehyde

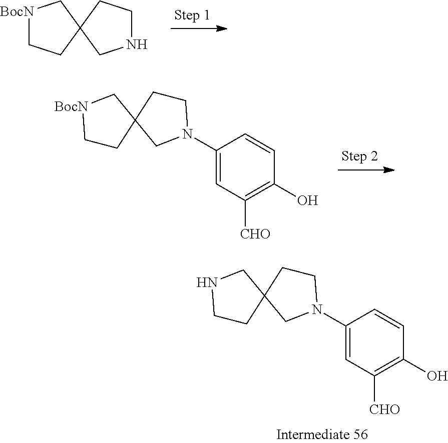

### Step 1: tert-Butyl 7-(3-formyl-4-hydroxyphenyl)-2,7-diazaspiro[4.4]nonane-2-carboxylate

A mixture of tert-butyl 2,7-diazaspiro[4.4]nonane-2-carboxylate (500 mg, 2.2 mmol), 5-bromo-2-hydroxybenzaldehyde (533 mg, 2.7 mmol), t-BuONa (2 M, 1.1 mL), t-BuXPhos-Pd-G3 (5 mg, 0.2 mmol) in 1,4-dioxane (2 mL) was degassed and purged with N₂ three times. The mixture was stirred at 90 °C for 16 hr under N₂ atmosphere. The reaction mixture was diluted with H₂O (40 mL) and extracted with EtOAc (20 mL×2). The combined organic layers were washed with brine (30 mL), dried over Na₂SO₄, filtered, and concentrated under reduced pressure. After purified by silica gel flash chromatography (Eluent of 0~30% EtOAc in petroleum ether), tert-butyl 7-(3-formyl-4-hydroxyphenyl)-2,7-diazaspiro[4.4]nonane-2-carboxylate (158 mg, yield: 26%) was obtained as a yellow oil. ¹H NMR (400 MHz, Chloroform-d) δ 10.33 (s, 1H), 9.87 (s, 1H), 6.96-6.89 (m, 1H), 6.86-6.81 (m, 1H), 6.67-6.58 (m, 1H), 3.52-3.43 (m, 2H), 3.42-3.31 (m, 4H), 3.26-3.20 (m, 2H), 2.06-1.89 (m, 4H), 1.46 (s, 9H).

### Step 2: 2-Hydroxy-5-(2,7-diazaspiro[4.4]nonan-2-yl)benzaldehyde

To a solution of tert-butyl 7-(3-formyl-4-hydroxy-phenyl)-2,7-diazaspiro[4.4]nonane-2-carboxylate (Intermediate 56, 198 mg, 572 μmol) in CH₂Cl₂ (2 mL) was added 4M HCl in EtOAc (2 mL). The mixture was stirred at 25 °C for 1 hr. The reaction mixture was concentrated under reduced pressure to give 2-hydroxy-5-(2,7-diazaspiro[4.4]nonan-2-yl)benzaldehyde (Intermediate 56: 160 mg, HCl salt) as a yellow solid. MS: m/z=247.0 [M+H]⁺.

### Intermediate 57: 3-(3-(4-((2,8-Diazaspiro[4.5]decan-2-yl)methyl)phenyl)-5-phenyl-3H-imidazo[4,5-b]pyridin-2-yl)pyridin-2-amine

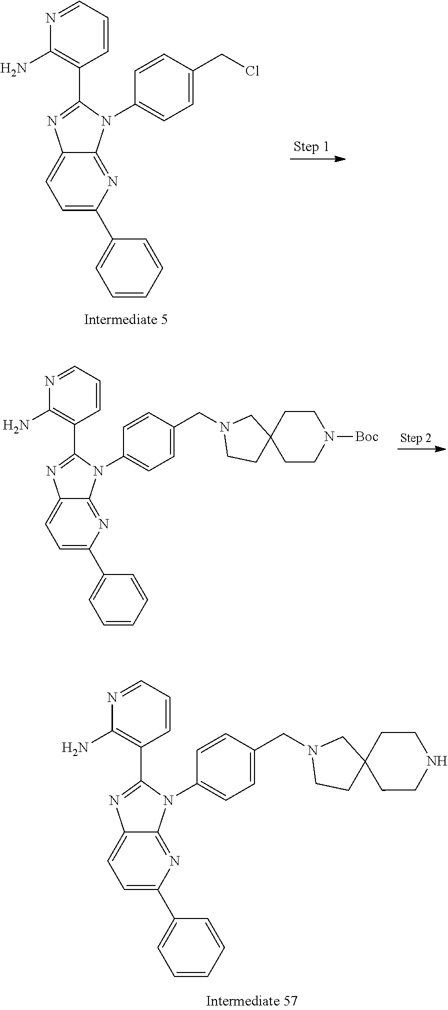 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)CC1 ; Nc1ncccc1-c1nc2ccc(-c3ccccc3)nc2n1-c1ccc(CN2CCC3(CCNCC3)C2)cc1]

Following the general procedures described in Intermediate 37, the reactions were carried out using corresponding starting materials. 3-(3-(4-((2,8-Diazaspiro[4.5]decan-2-yl)methyl)phenyl)-5-phenyl-3H-imidazo[4,5-b]pyridin-2-yl)pyridin-2-amine (Intermediate 57, 600 mg, HCl salt, yield: 91%) was obtained as a yellow solid. MS: m/z=516.3 [M+H]^{t}. ¹H NMR (400 MHz, Methanol-di) δ 8.33 (d, J=8.8 Hz, 1H), 8.10-8.01 (m, 4H), 7.96-7.87 (m, 3H), 7.72 (d, J=8.0 Hz, 2H), 7.49-7.40 (m, 3H), 6.96-6.87 (m, 1H), 4.67-4.54 (m, 2H), 3.75-3.65 (m, 2H), 3.51 (d, J=10.0 Hz, 1H), 3.35 (s, 2H), 3.32-3.20 (m, 3H), 2.32-2.23 (m, 1H), 2.15-2.05 (m, 2H), 2.04-1.96 (m, 3H).

### Intermediate 58: 2-Hydroxy-4-(2,8-diazaspiro[4.5]decan-8-yl)benzaldehyde

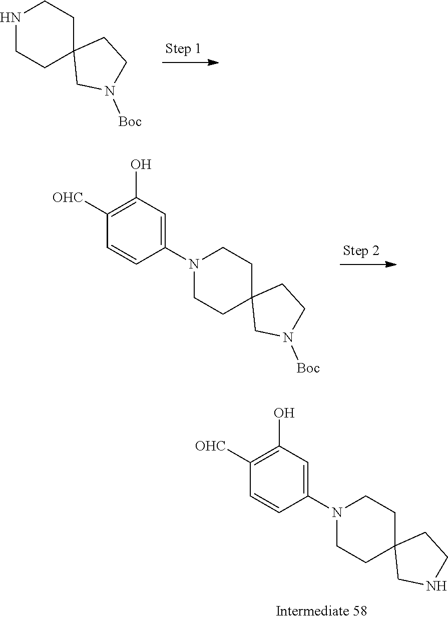

Following the general procedures described in Intermediate 48, the reactions were carried out using corresponding starting materials. 2-Hydroxy-4-(2,8-diazaspiro[4.5]decan-8-yl)benzaldehyde (Intermediate 58, 180 mg, crude) was obtained as a black brown solid. MS: m/z=261.1 [M+H]⁺.

### Intermediate 59: 3-(3-(4-((2,7-Diazaspiro[4.5]decan-2-yl)methyl)phenyl)-5-phenyl-3H-imidazo[4,5-b]pyridin-2-yl)pyridin-2-amine

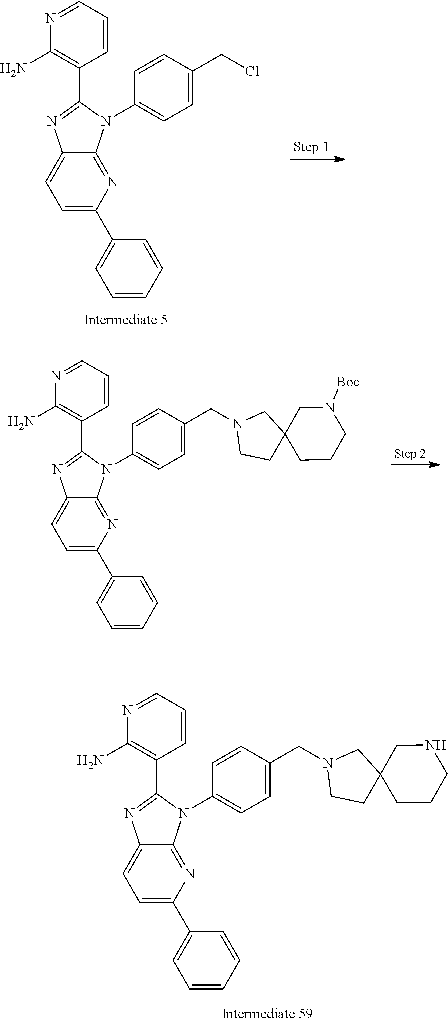 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCCNC3)C2)cc1]

Following the general procedures described in Intermediate 37, the reactions were carried out using corresponding starting materials. 3-(3-(4-((2,7-Diazaspiro[4.5]decan-2-yl)methyl)phenyl)-5-phenyl-3H-imidazo[4,5-b]pyridin-2-yl)pyridin-2-amine (Intermediate 59, 188 mg, HCl salt, yield: 88%) was obtained as a yellow solid. MS: m/z=516.4 [M+H]⁺. ¹H NMR (400 MHz, Methanol-d₄) δ 8.32 (d, J=8.4 Hz, 1H), 8.09-8.00 (m, 4H), 7.93 (d, J=8.4 Hz, 2H), 7.89 (d, J=7.2 Hz, 1H), 7.72 (d, J=8.0 Hz, 2H), 7.49-7.37 (m, 3H), 6.99-6.85 (m, 1H), 4.62 (d, J=11.6 Hz, 2H), 3.78-3.72 (m, 1H), 3.65-3.47 (m, 2H), 3.44-3.38 (m, 1H), 3.35 (s, 2H), 3.18-3.13 (m, 2H), 2.26-2.13 (m, 1H), 2.11-2.01 (m, 1H), 1.95-1.86 (m, 4H).

### Intermediate 60: 2-Hydroxy-4-(2,7-diazaspiro[4.5]decan-7-yl)benzaldehyde

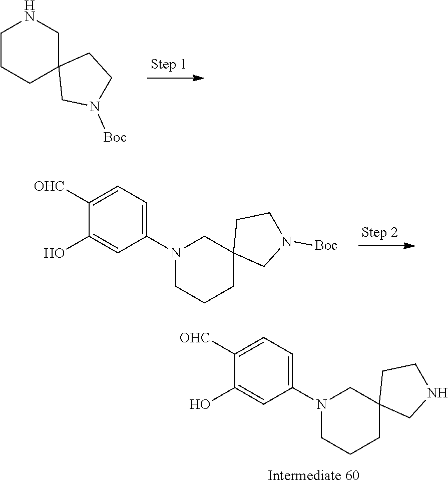

Following the general procedures described in Intermediate 48, the reactions were carried out using corresponding starting materials. 2-Hydroxy-4-(2,7-diazaspiro[4.5]decan-7-yl)benzaldehyde (Intermediate 60, 200 mg, HCl salt) was obtained as a black brown solid. MS: m/z=261.1 [M+H]⁺.

### Intermediate 61: 2-Hydroxy-4-(2,6-diazaspiro[3.3]heptan-2-yl)benzaldehyde

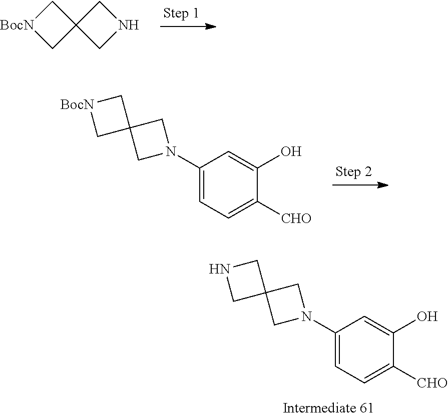 [SMILES: CC(C)(C)OC(=O)N1CC2(CNC2)C1 ; CC(C)(C)OC(=O)N1CC2(C1)CN(c1ccc(C=O)c(O)c1)C2 ; O=Cc1ccc(N2CC3(CNC3)C2)cc1O]

Following the general procedures described in Intermediate 48, the reactions were carried out using corresponding starting materials. 2-Hydroxy-4-(2,6-diazaspiro[3.3]heptan-2-yl)benzaldehyde (Intermediate 61, 106 mg, TFA salt, crude) was obtained as a yellow oil. MS: m/z=219.1 [M+H]⁺.

### Intermediate 62: 2-hydroxy-5-(2,6-diazaspiro[3.3]heptan-2-yl)benzaldehyde

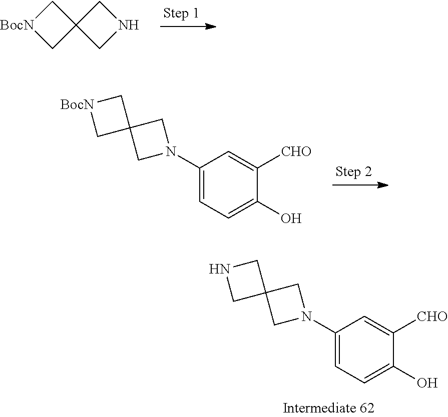 [SMILES: CC(C)(C)OC(=O)N1CC2(CNC2)C1 ; CC(C)(C)OC(=O)N1CC2(C1)CN(c1ccc(O)c(C=O)c1)C2 ; O=Cc1cc(N2CC3(CNC3)C2)ccc1O]

### Step 1: tert-Butyl 6-(3-formyl-4-hydroxyphenyl)-2,6-diazaspiro[3.3]heptane-2-carboxylate

To a mixture of tert-butyl 2,6-diazaspiro[3.3]heptane-2-carboxylate (200 mg, 1.0 mmol) and 5-bromo-2-hydroxybenzaldehyde (203 mg, 1.0 mmol) in 1,4-dioxane (5 mL) were added XPhos Pd G4 (87 mg, 101 μmol) and Cs₂CO₃ (986 mg, 3.0 mmol) at 25 °C The mixture was degassed and purged with N₂ three times. The mixture was stirred at 100 °C for 12 hr under N₂ atmosphere. The reaction mixture was diluted with CH₂Cl₂ (20 mL) and washed with H₂O (20 mL×2) and brine (20 mL×2). The combined organic phases were dried over Na₂SO₄, filtered and concentrated under reduced pressure. After purified by silica gel flash chromatography (Eluent of 0~30% EtOAc in petroleum ether), tert-butyl 6-(3-formyl-4-hydroxyphenyl)-2,6-diazaspiro[3.3]heptane-2-carboxylate (100 mg, yield: 29%) was obtained as a yellow solid. MS: m/z=319.1 [M+H]⁺. ¹H NMR (400 MHz, Chloroform-d) δ 11.97 (s, 1H), 7.32-7.25 (m, 1H), 7.19 (d, J=8.0 Hz, 1H), 6.89 (d, J=8.4 Hz, 1H), 6.77-6.67 (m, 1H), 4.69-4.15 (m, 4H), 4.01 (s, 4H), 1.36 (s, 9H).

### Step 2: 2-Hydroxy-5-(2,6-diazaspiro[3.3]heptan-2-yl)benzaldehyde

To a solution of tert-butyl 6-(3-formyl-4-hydroxyphenyl)-2,6-diazaspiro[3.3]heptane-2-carboxylate (100 mg, 314 μmol) in CH₂Cl₂ (3 mL) was added TFA (1.8 g, 13.5 mmol) at 25 °C The mixture was stirred at 25 °C for 1 hr. The reaction mixture was concentrated under reduced pressure to give 2-hydroxy-5-(2,6-diazaspiro[3.3]heptan-2-yl)benzaldehyde (Intermediate 62, 106 mg, TFA salt, crude) as a yellow oil. MS: m/z=219.0 [M+H]⁺.

### Intermediate 63: 3-(3-(4-((2,6-diazaspiro[3.4]octan-2-yl)methyl)phenyl)-5-phenyl-3H-imidazo[4,5-b]pyridin-2-yl)pyridin-2-amine

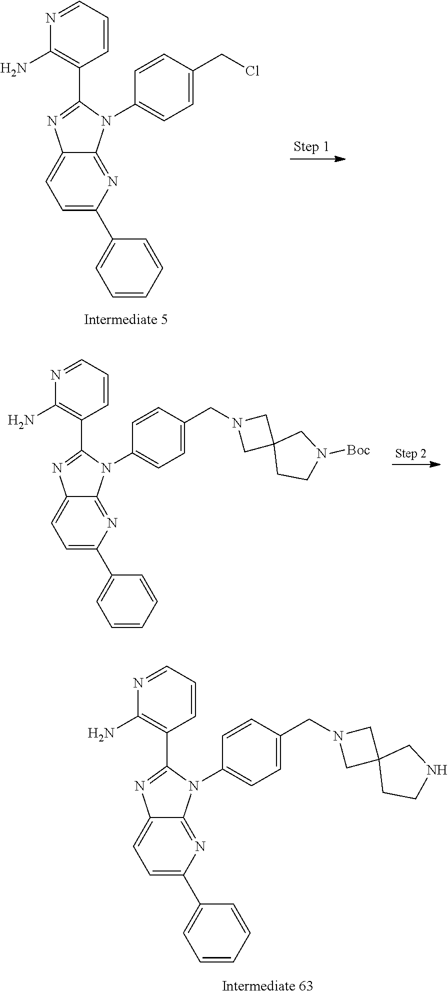

Following the general procedures described in Intermediate 37, the reactions were carried out using corresponding starting materials. 3-(3-(4-((2,6-Diazaspiro[3.4]octan-2-yl)methyl)phenyl)-5-phenyl-3H-imidazo[4,5-b]pyridin-2-yl)pyridin-2-amine (Intermediate 63, 104 mg, yield: 14%) was obtained as a yellow solid. MS: m/z=488.3 [M+H]⁺. 1 H NMR (400 MHz, Dimethylsulfoxide-d₆) δ 8.26 (d, J=8.4 Hz, 1H), 8.06-7.95 (m, 4H), 7.51-7.36 (m, 7H), 7.15 (dd, J=7.6, 1.6 Hz, 1H), 6.99 (br s, 2H), 6.39 (dd, J=7.6, 4.8 Hz, 1H), 3.63 (s, 2H), 3.16-3.10 (m, 4H), 2.88 (s, 2H), 2.74 (t, J=6.8 Hz, 2H), 1.82 (t, J=6.8 Hz, 2H), 1.25-1.22 (m, 1H).

### Intermediate 64: 2-Hydroxy-4-(2,6-diazaspiro[3.4]octan-6-yl)benzaldehyde

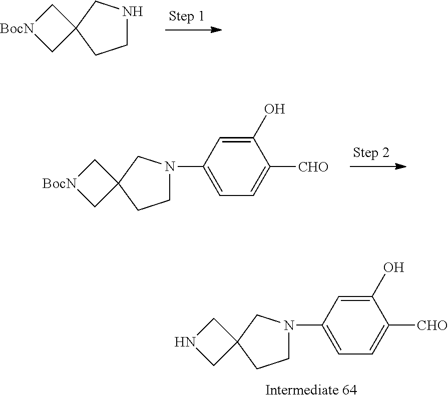 [SMILES: CC(C)(C)OC(=O)N1CC2(CCNC2)C1 ; CC(C)(C)OC(=O)N1CC2(CCN(c3ccc(C=O)c(O)c3)C2)C1 ; O=Cc1ccc(N2CCC3(CNC3)C2)cc1O]

Following the general procedures described in Intermediate 48, the reactions were carried out using corresponding starting materials. 2-Hydroxy-4-(2,6-diazaspiro[3.4]octan-6-yl)benzaldehyde (Intermediate 64, 102 mg, TFA salt) was obtained as an off white solid. MS: m/z=233.1 [M+H]⁺.

### Intermediate 65: 3-(3-(4-((2,7-Diazaspiro[3.5]nonan-2-yl)methyl)phenyl)-5-phenyl-3H-imidazo[4,5-b]pyridin-2-yl)pyridin-2-amine

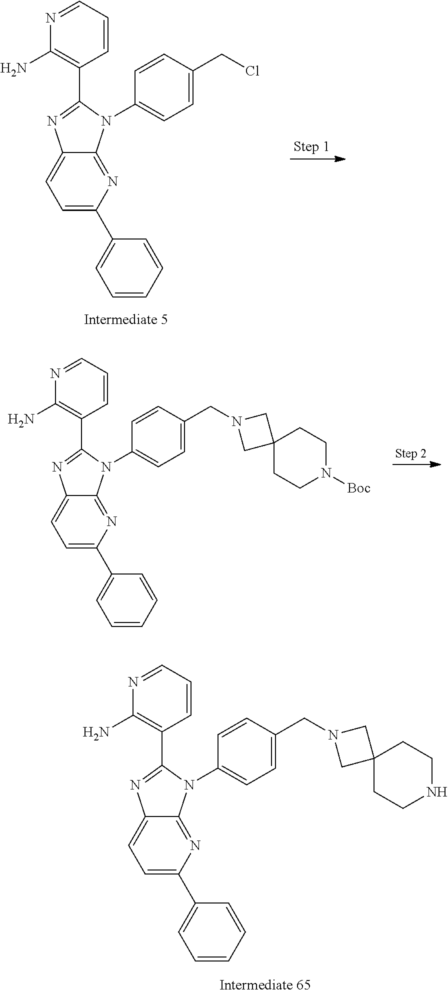

Following the general procedures described in Intermediate 37, the reactions were carried out using corresponding starting materials. 3-(3-(4-((2,7-Diazaspiro[3.5]nonan-2-yl)methyl)phenyl)-5-phenyl-3H-imidazo[4,5-b]pyridin-2-yl)pyridin-2-amine (Intermediate 65, 270 mg, HCl salt, yield: 86%) as a yellow solid. MS: m/z=502.3 [M+H]⁺. ¹H NMR (400 MHz, Dimethylsulfoxide-d₆) δ 11.79-11.63 (m, 1H), 9.00-8.82 (m, 2H), 8.36 (d, J=8.8 Hz, 1H), 8.33-8.18 (m, 1H), 8.13 (dd, J=6.8, 1.2 Hz, 1H), 8.04-8.08 (m, 3H), 7.83-7.75 (m, 3H), 7.65 (d, J=8.4 Hz, 2H), 7.52-7.46 (m, 2H), 7.45-7.39 (m, 1H), 6.92-6.83 (m, 1H), 4.50 (d, J=6.0 Hz, 2H), 3.96 (d, J=6.0 Hz, 4H), 3.07-3.10 (m, 2H), 2.95-3.02 (m, 2H), 2.10-2.17 (m, 2H), 1.97-2.04 (m, 2H).

### Intermediate 66: 2-Hydroxy-4-(2,7-diazaspiro[3.5]nonan-7-yl)benzaldehyde

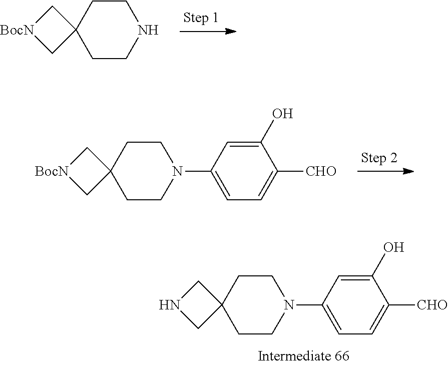 [SMILES: CC(C)(C)OC(=O)N1CC2(CCNCC2)C1 ; CC(C)(C)OC(=O)N1CC2(CCN(c3ccc(C=O)c(O)c3)CC2)C1 ; O=Cc1ccc(N2CCC3(CC2)CNC3)cc1O]

Following the general procedures described in Intermediate 48, the reactions were carried out using corresponding starting materials. 2-hydroxy-4-(2,7-diazaspiro[3.5]nonan-7-yl)benzaldehyde (Intermediate 66, 150 mg, HCl salt, yield: 80%) was obtained as a brown solid. MS: m/z=247.1 [M+H]⁺.

### Intermediate 67: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

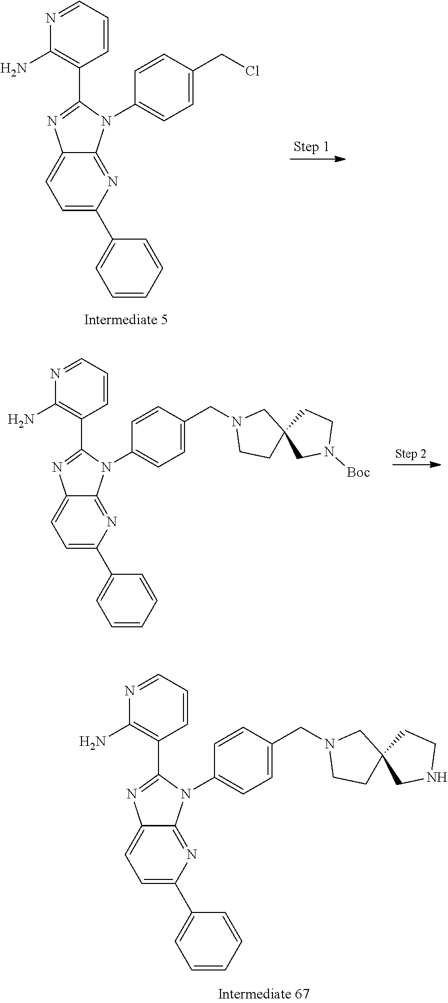 [SMILES: Nc1ncccc1-c1nc2ccc(-c3ccccc3)nc2n1-c1ccc(CCl)cc1 ; CC(C)(C)OC(=O)N1CC[C@@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@@]3(CCNC3)C2)cc1]

### Step 1: tert-Butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 5 (1.0 g, 2.43 mmol) in DMF (10 mL) were added K₂CO₃ (671 mg, 4.86 mmol) and tert-butyl (R)-2,7-diazaspiro[4.4]nonane-2-carboxylate (604 mg, 2.67 mmol). The mixture was stirred at 25 °C for 16 hr. The reaction mixture was diluted with H₂O (10 mL) at 25 °C and extracted with CH₂Cl₂ (20 mL×3). The combined organic layers were washed with brine (30 ml), dried over Na₂SO₄, filtered, and concentrated under reduced pressure. The crude product was purified by silica gel flash chromatography (Eluent of 1-5% MeOH in CH₂Cl₂) to give tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (920 mg, yield: 63%) as a yellow solid. MS: m/z=602.6 [M+H]⁺.

### Step 2: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

A solution of tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (700 mg, 1.16 mmol) in 4 M HCl/1,4-dioxane (7 mL) was stirred at 25 °C for 2 hr. The reaction was concentrated under reduced pressure to give (R)-3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 67, 600 mg, HCl salt, yield: 96%) as a yellow solid. MS: m/z=502.3 [M+H]⁺. ¹H NMR (400 MHz, Dimethylsulfoxide-d₆) δ 12.00-11.77 (m, 1H), 9.73-9.52 (m, 2H), 8.58-8.44 (m, 1H), 8.38 (d, J=8.4 Hz, 1H), 8.16 (dd, J=6.0, 1.6 Hz, 1H), 8.04-8.09 (m, 3H), 7.92-7.85 (m, 3H), 7.66 (d, J=8.4 Hz, 2H), 7.52-7.46 (m, 2H), 7.45-7.40 (m, 1H), 7.00-6.92 (m, 1H), 4.57-4.41 (m, 2H), 3.50-3.58 (m, 4H), 3.41-3.33 (m, 2H), 3.25-3.22 (m, 2H), 2.28-2.13 (m, 2H), 2.10-1.97 (m, 2H).

### Intermediate 68: (R)-2-Hydroxy-4-(2,7-diazaspiro[4.4]nonan-2-yl)benzaldehyde

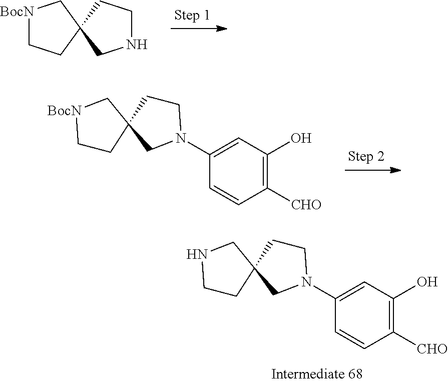

Following the general procedures described in Intermediate 48, the reactions were carried out using corresponding starting materials. (R)-2-hydroxy-4-(2,7-diazaspiro[4.4]nonan-2-yl)benzaldehyde (Intermediate 68, 245 mg, HCl salt) was obtained as a yellow solid. MS: m/z=247.0 [M+H]⁺.

### Intermediate 69: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

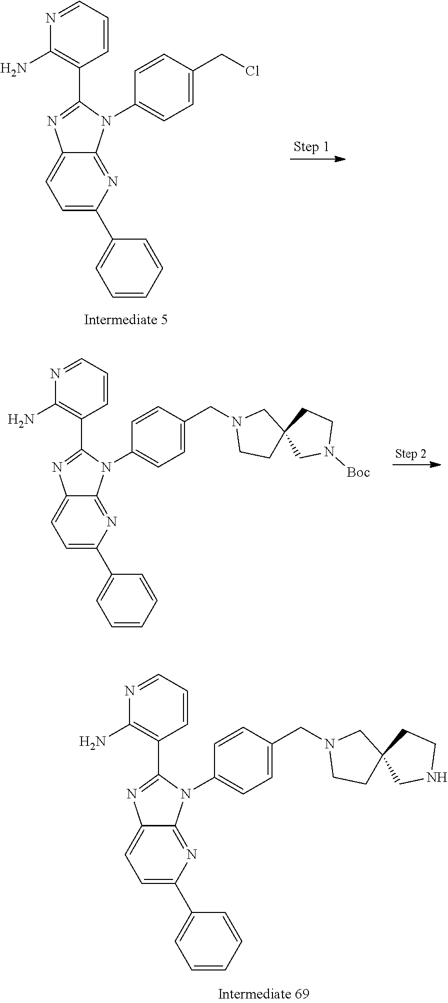 [SMILES: Nc1ncccc1-c1nc2ccc(-c3ccccc3)nc2n1-c1ccc(CCl)cc1 ; CC(C)(C)OC(=O)N1CC[C@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@]3(CCNC3)C2)cc1]

Following the general procedures described in Intermediate 67, the reactions were carried out using corresponding starting materials. (S)-3-(3-(4-((2,7-Diazaspiro[4.4]nonan-2-yl)methyl)phenyl)-5-phenyl-3H-imidazo[4,5-b]pyridin-2-yl)pyridin-2-amine (Intermediate 69, 500 mg, HCl salt, yield: 96%) was obtained as a yellow solid. MS: m/z=502.3 [M+H]⁺. ¹H NMR (400 MHz, Dimethylsulfoxide-d) δ 11.9 (br s, 1H), 9.80-9.66 (m, 2H), 8.66-8.50 (m, 2H), 8.38 (d, J=8.4 Hz, 1H), 8.17 (dd, J=6.4, 1.6 Hz, 1H), 8.08-8.05 (m, 3H), 7.93-7.86 (M, 3H), 7.66 (d, J=8.4 Hz, 2H), 7.54-7.40 (m, 3H), 7.00-6.95 (m, 1H), 3.55-3.50 (m, 1H), 3.40-3.17 (m, 8H), 2.33-1.95 (m, 5H).

### Intermediate 70: (S)-2-Hydroxy-4-(2,7-diazaspiro[4.4]nonan-2-yl)benzaldehyde

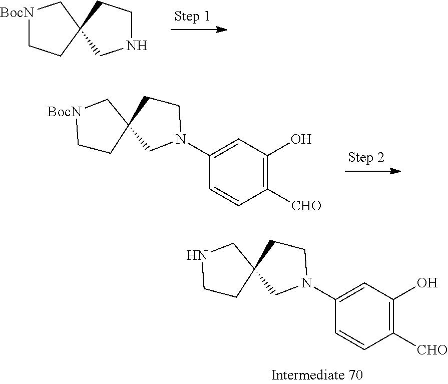

Following the general procedures described in Intermediate 48, the reactions were carried out using corresponding starting materials. (S)-2-Hydroxy-4-(2,7-diazaspiro[4.4]nonan-2-yl)benzaldehyde (Intermediate 70, 160 mg, yield: 98%, HCl) was obtained as yellow oil, which was used in next step without further purification. MS: m/z=247.3 [M+H]⁺.

### Intermediate 71: (R)-2-hydroxy-4-(2-methylpiperazin-1-yl)benzaldehyde

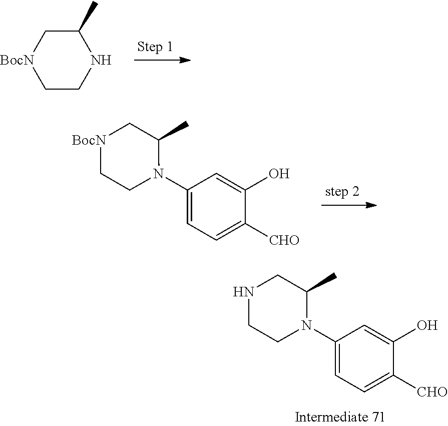

Following the general procedures described in Intermediate 48, the reactions were carried out using corresponding starting materials. (R)-2-Hydroxy-4-(2-methylpiperazin-1-yl)benzaldehyde (Intermediate 71, 128 mg, crude) was obtained as a brown solid, which was used into next step without further purification.

### Intermediate 72: (S)-3-(3-(4-((3-methylpiperazin-1-yl)methyl)phenyl)-5-phenyl-3H-imidazo[4,5-b]pyridin-2-yl)pyridin-2-amine

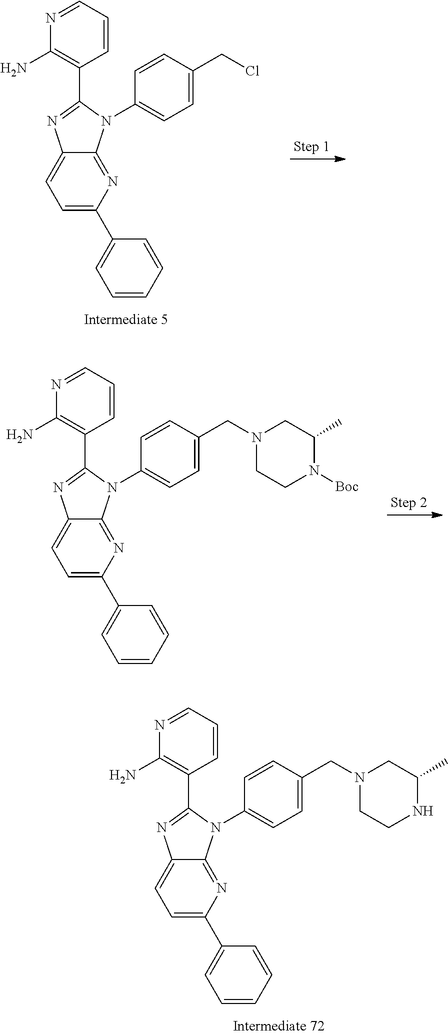 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)CCN1C(=O)OC(C)(C)C ; C[C@H]1CN(Cc2ccc(-n3c(-c4cccnc4N)nc4ccc(-c5ccccc5)nc43)cc2)CCN1]

Following the general procedures described in Intermediate 67, the reactions were carried out using corresponding starting materials. (S)-3-(3-(4-((3-Methylpiperazin-1-yl)methyl)phenyl)-5-phenyl-3H-imidazo[4,5-b]pyridin-2-yl)pyridin-2-amine (Intermediate 72, 350 mg, yield: 82%) was obtained as a yellow solid. MS: m/z=476.2 [M+H]⁺. ¹H NMR (400 MHz, Methanol-d₄) δ 8.32 (d, J=8.4 Hz, 1H), 8.07-7.99 (m, 4H), 7.94-7.84 (m, 3H), 7.73 (d, J=8.4 Hz, 2H), 7.50-7.37 (m, 3H), 6.96-6.88 (m, 1H), 4.57 (s, 1H), 4.60-4.53 (s, 2H), 3.94-3.83 (m, 1H), 3.81-3.69 (m, 3H), 3.68-3.56 (m, 1H), 3.54-3.41 (m, 1H), 3.39-3.31 (m, 1H), 1.45 (d, J=6.8 Hz, 3H).

### Intermediate 73: (S)-2-Hydroxy-4-(2-methylpiperazin-1-yl)benzaldehyde

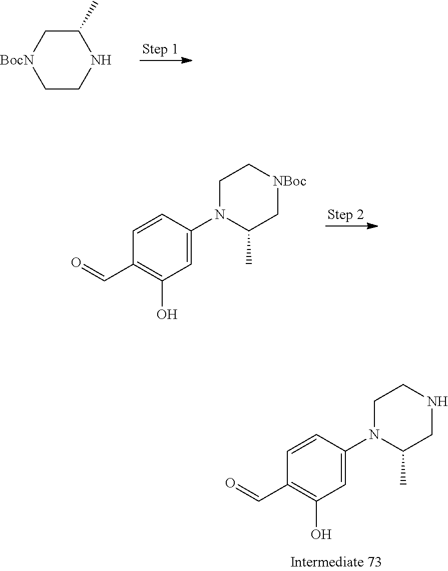 [SMILES: C[C@H]1CN(C(=O)OC(C)(C)C)CCN1 ; C[C@H]1CN(C(=O)OC(C)(C)C)CCN1c1ccc(C=O)c(O)c1 ; C[C@H]1CNCCN1c1ccc(C=O)c(O)c1]

Following the general procedures described in Intermediate 48, the reactions were carried out using corresponding starting materials. (S)-2-hydroxy-4-(2-methylpiperazin-1-yl)benzaldehyde (Intermediate 73, 100 mg, yield: 83%, HCl) was obtained as yellow oil. MS: m/z=221.2 [M+H]⁺.

### Intermediate 74: 3-(3-(4-((3,8-diazabicyclo[3.2.1]octan-3-yl)methyl)phenyl)-5-phenyl-3H-imidazo[4,5-b]pyridin-2-yl)pyridin-2-amine

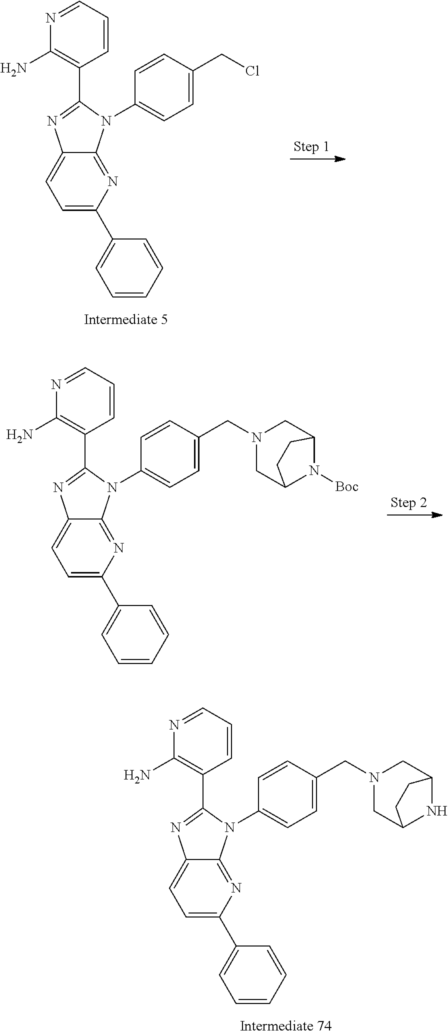 [SMILES: Nc1ncccc1-c1nc2ccc(-c3ccccc3)nc2n1-c1ccc(CCl)cc1 ; CC(C)(C)OC(=O)N1C2CCC1CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3CCC(C2)N3)cc1]

Following the general procedures described in Intermediate 67, the reactions were carried out using corresponding starting materials. 3-(3-(4-((3,8-Diazabicyclo[3.2.1]octan-3-yl)methyl)phenyl)-5-phenyl-3H-imidazo[4,5-b]pyridin-2-yl)pyridin-2-amine (Intermediate 74, 240 mg, HCl salt) as a light yellow solid. MS: m/z=488.1 [M+H]⁺. ¹H NMR (400 MHz, Methanol-d₄) δ 8.34-8.28 (m, 1H), 8.05-7.95 (m, 4H), 7.90-7.85 (m, 3H), 7.65 (d, J=8.0 Hz, 2H), 7.48-7.37 (m, 3H), 6.90 (t, J=6.8 Hz, 1H), 4.35-4.25 (m, 4H), 3.50-3.40 (m, 4H), 2.41 (d, J=8.4 Hz, 2H), 2.25-2.16 (m, 2H).

### Intermediate 75: 4-(3,8-Diazabicyclo[3.2.1]octan-8-yl)-2-hydroxybenzaldehyde

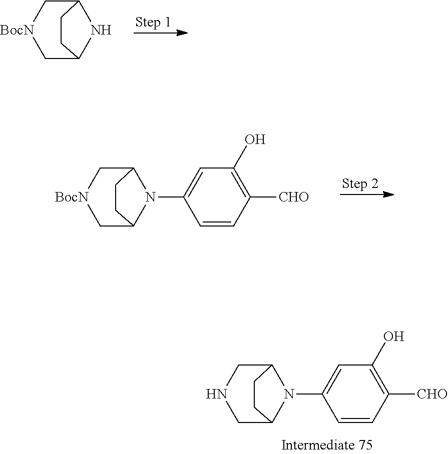 [SMILES: CC(C)(C)OC(=O)N1CC2CCC(C1)N2 ; CC(C)(C)OC(=O)N1CC2CCC(C1)N2c1ccc(C=O)c(O)c1 ; O=Cc1ccc(N2C3CCC2CNC3)cc1O]

Following the general procedures described in Intermediate 48, the reactions were carried out using corresponding starting materials. 4-(3,8-Diazabicyclo[3.2.1]octan-8-yl)-2-hydroxybenzaldehyde (Intermediate 75, 500 mg, HCl salt, yield: 91%) was obtained as a yellow solid. MS: m/z=233.1 [M+H]⁺.

### Intermediate 76: 3-(3-(4-((3,8-Diazabicyclo[3.2.1]octan-8-yl)methyl)phenyl)-5-phenyl-3H-imidazo[4,5-b]pyridin-2-yl)pyridin-2-amine

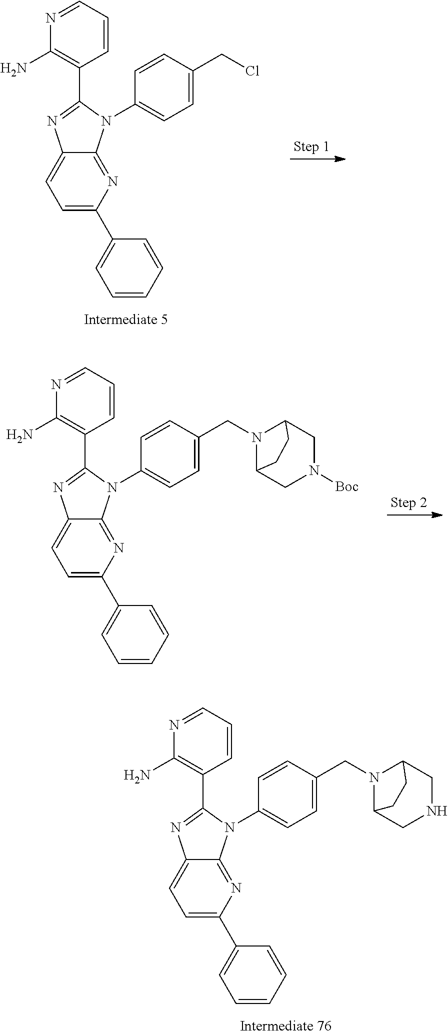 [SMILES: Nc1ncccc1-c1nc2ccc(-c3ccccc3)nc2n1-c1ccc(CCl)cc1 ; CC(C)(C)OC(=O)N1CC2CCC(C1)N2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C3CCC2CNC3)cc1]

Following the general procedures described in Intermediate 37, the reactions were carried out using corresponding starting materials. 3-(3-(4-((3,8-Diazabicyclo[3.2.1]octan-8-yl)methyl)phenyl)-5-phenyl-3H-imidazo[4,5-b]pyridin-2-yl)pyridin-2-amine (Intermediate 76, 535 mg, HCl salt) was obtained as a light yellow solid. MS: m/z=488.2 [M+H]⁺. ¹H NMR (400 MHz, Dimethylsulfoxide-d₆) δ 12.42 (br s, 1H), 10.4-10.0 (m, 2H), 8.55 (br s, 1H), 8.38 (d, J=8.0 Hz, 1H), 8.13-8.07 (m, 1H), 8.06-8.00 (m, 5H), 7.98-7.94 (m, 1H), 7.69 (d, J=8.0 Hz, 2H), 7.50-7.40 (m, 3H), 6.94 (t, J=7.2 Hz, 1H), 4.39-4.33 (m, 2H), 4.01 (s, 2H), 3.95-3.89 (m, 3H), 3.42-3.39 (m, 3H), 2.47-2.38 (m, 2H).

### Intermediate 77: 4-(3,8-Diazabicyclo[3.2.1]octan-3-yl)-2-hydroxybenzaldehyde

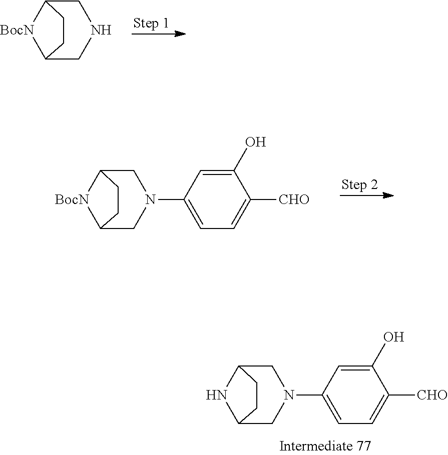 [SMILES: CC(C)(C)OC(=O)N1C2CCC1CNC2 ; CC(C)(C)OC(=O)N1C2CCC1CN(c1ccc(C=O)c(O)c1)C2 ; O=Cc1ccc(N2CC3CCC(C2)N3)cc1O]

Following the general procedures described in Intermediate 48, the reactions were carried out using corresponding starting materials. 4-(3,8-diazabicyclo[3.2.1]octan-3-yl)-2-hydroxybenzaldehyde (Intermediate 77, 500 mg, HCl salt, yield: 91%) was obtained as a yellow solid. MS: m/z=233.1 [M+H]⁺.

### Intermediate 78: 1-(4-(2-(2-aminopyridin-3-yl)-5-phenyl-3H-imidazo[4,5-b]pyridin-3-yl)benzyl)azepan-4-amine

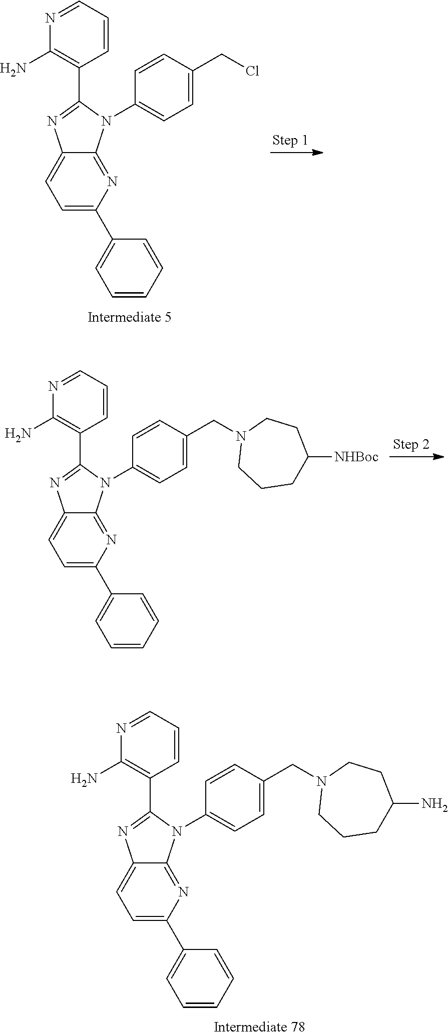 [SMILES: Nc1ncccc1-c1nc2ccc(-c3ccccc3)nc2n1-c1ccc(CCl)cc1 ; CC(C)(C)OC(=O)NC1CCCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCCC(N)CC2)cc1]

### Step 1: tert-Butyl (1-(4-(2-(2-aminopyridin-3-yl)-5-phenyl-3H-imidazo[4,5-b]pyridin-3-yl)benzyl)azepan-4-yl)carbamate

To a solution of Intermediate 5 (1.0 g, 2.43 mmol) and tert-butyl N-(azepan-4-yl)carbamate (670 mg, 2.67 mmol) in DMF (10 mL) was added DIEA (1.26 g, 9.71 mmol). The mixture was stirred at 80 °C for 16 hr. The mixture was quenched with H₂O (40 mL) and extracted with EtOAc (40 mL×3). The combined organic phase was dried over anhydrous Na₂SO₄, filtered and concentrated. The residue was purified by silica gel flash chromatography (Eluent of 0~10% MeOH in CH₂C2) to give tert-butyl (1-(4-(2-(2-aminopyridin-3-yl)-5-phenyl-3H-imidazo[4,5-b]pyridin-3-yl)benzyl)azepan-4-yl)carbamate (890 mg, yield: 62%) as a yellow solid. MS: m/z=590.4 [M+H]⁺. ¹H NMR (400 MHz, Chloroform-d) 58.13 (d, J=8.4 Hz, 1H), 8.09-8.05 (m, 1H), 8.02 (d, J=7.6 Hz, 2H), 7.80 (d, J=8.4 Hz, 1H), 7.55-7.47 (m, 2H), 7.47-7.33 (m, 5H), 7.15-7.06 (m, 1H), 6.61 (br s, 2H), 6.35 (dd, J=7.6, 4.8 Hz, 1H), 5.08-4.93 (m, 1H), 3.99-3.83 (m, 1H), 3.73 (s, 2H), 2.88-2.68 (m, 2H), 2.67-2.52 (m, 2H), 2.00-1.68 (m, 6H), 1.43 (s, 9H).

### Step 2: 1-(4-(2-(2-Aminopyridin-3-yl)-5-phenyl-3H-imidazo[4,5-b]pyridin-3-yl)benzyl)azepan-4-amine

A solution of tert-butyl (1-(4-(2-(2-aminopyridin-3-yl)-5-phenyl-3H-imidazo[4,5-b]pyridin-3-yl)benzyl)azepan-4-yl)carbamate (100 mg, 170 μmol) in HCl in 1,4-dioxane (4M, 1 mL) was stirred at 25 °C for 1 hr. The mixture was filtered to give 1-(4-(2-(2-aminopyridin-3-yl)-5-phenyl-3H-imidazo[4,5-b]pyridin-3-yl)benzyl)azepan-4-amine (Intermediate 78, 80 mg HCl salt, yield: 89%) as a yellow solid. MS: m/z=490.2 [M+H]r. ¹H NMR (400 MHz, Dimethylsulfoxide-d₆) δ 11.56-11.29 (m, 1H), 8.37 (J=8.4 Hz, 1H), 8.36-8.18 (m, 3H), 8.14 (J=7.6 Hz, 1H), 8.12-7.97 (m, 3H), 7.96-7.80 (m, 3H), 7.67 (d, J=8.4 Hz, 2H), 7.52-7.38 (m, 3H), 6.93-6.84 (m, 1H), 4.52-4.33 (m, 2H), 3.40-3.38 (m, 2H), 3.21-3.12 (m, 2H), 3.09-2.96 (m, 1H), 2.26-1.98 (m, 4H), 1.93-1.80 (m, 1H), 1.75-1.61 (m, 1H).

### Intermediate 79: 1-(4-(2-(2-Aminopyridin-3-yl)-5-phenyl-3H-imidazo[4,5-b]pyridin-3-yl)benzyl)azepan-3-amine

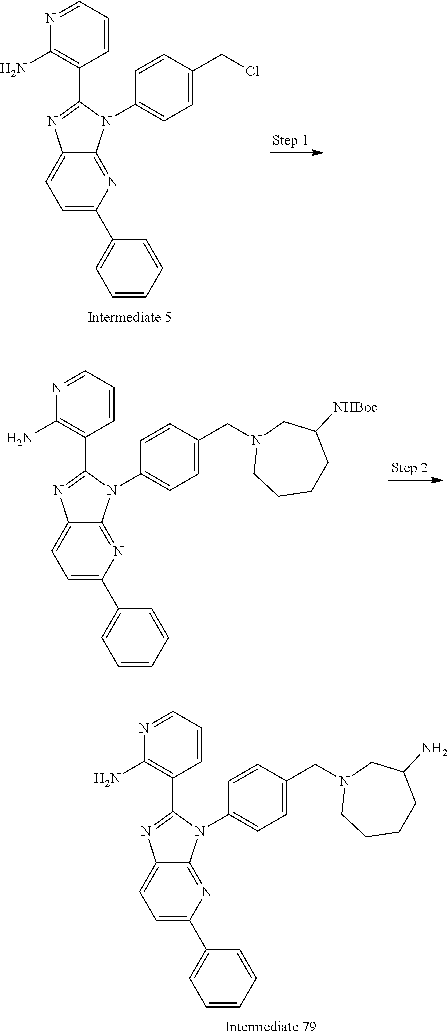 [SMILES: Nc1ncccc1-c1nc2ccc(-c3ccccc3)nc2n1-c1ccc(CCl)cc1 ; CC(C)(C)OC(=O)NC1CCCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCCCC(N)C2)cc1]

Following the general procedures described in Intermediate 37, the reactions were carried out using corresponding starting materials. 1-(4-(2-(2-Aminopyridin-3-yl)-5-phenyl-3H-imidazo[4,5-b]pyridin-3-yl)benzyl)azepan-3-amine (Intermediate 79, 83 mg, HCl salt yield: 93%) was obtained as a yellow solid. MS: m/z=490.2 [M+H]⁺. ¹H NMR (400 MHz, Dimethylsulfoxide-d₆) δ 8.27 (d, J=8.4 Hz, 1H), 8.05-7.98 (m, 4H), 7.53-7.37 (m, 7H), 7.16-7.12 (m, 1H), 7.07 (br s, 2H), 6.38 (dd, J=7.6, 4.8 Hz, 1H), 3.77-3.72 (m, 2H), 3.62-3.53 (m, 1H), 2.89-2.78 (m, 2H), 2.65-2.61 (m, 2H), 2.59-2.53 (m, 2H), 1.82-1.73 (m, 1H), 1.67-1.48 (m, 4H), 1.46-1.25 (m, 1H).

### Intermediate 81: 4-(1,4-Diazepan-1-yl)-2-hydroxybenzaldehyde

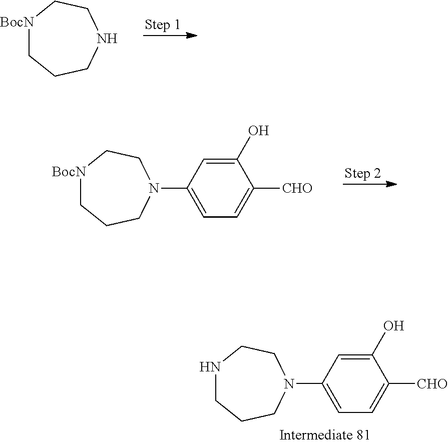 [SMILES: CC(C)(C)OC(=O)N1CCCNCC1 ; CC(C)(C)OC(=O)N1CCCN(c2ccc(C=O)c(O)c2)CC1 ; O=Cc1ccc(N2CCCNCC2)cc1O]

Following the general procedures described in Intermediate 48, the reactions were carried out using corresponding starting materials. 4-(1,4-Diazepan-1-yl)-2-hydroxybenzaldehyde (Intermediate 81, 90 mg, HCl salt, yield: 74%) was obtained as a brown solid. MS: m/z=221.0 [M+H]⁺.

### Intermediate 82: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

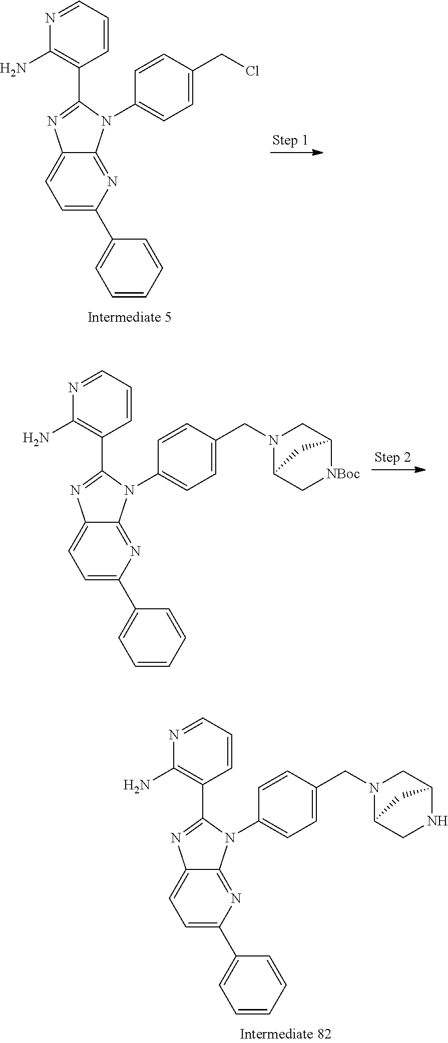 [SMILES: Nc1ncccc1-c1nc2ccc(-c3ccccc3)nc2n1-c1ccc(CCl)cc1 ; CC(C)(C)OC(=O)N1C[C@@H]2C[C@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@@H]3C[C@H]2CN3)cc1]

Following the general procedures described in Intermediate 78, the reactions were carried out using corresponding starting materials. 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine (Intermediate 82, 41 mg, HCl salt, yield: 95%) was obtained as a light-yellow solid. MS: m/z=474.2 [M+H]⁺. ¹H NMR (400 MHz, Dimethylsulfoxide-d₆) δ 12.43-11.57 (m, 1H), 10.39-9.51 (m, 2H), 8.55-8.39 (m, 1H), 8.38 (d, J=8.4 Hz, 1H), 8.14 (d, J=5.2 Hz, 1H), 8.11-8.01 (m, 3H), 7.94 (d, J=8.0 Hz, 2H), 7.84 (d, J=7.2 Hz, 1H), 7.69 (d, J=8.4 Hz, 2H), 7.54-7.40 (m, 3H), 6.91-6.87 (m, 1H), 4.68-4.40 (m, 4H), 3.99-3.94 (m, 1H), 3.80-3.75 (m, 2H), 3.42-3.40 (m, 1H), 2.60-2.52 (m, 1H), 2.20-2.06 (m, 1H).

### Intermediate 83: 4-((1R,4R)-2,5-Diazabicyclo[2.2.1]heptan-2-yl)-2-hydroxybenzaldehyde

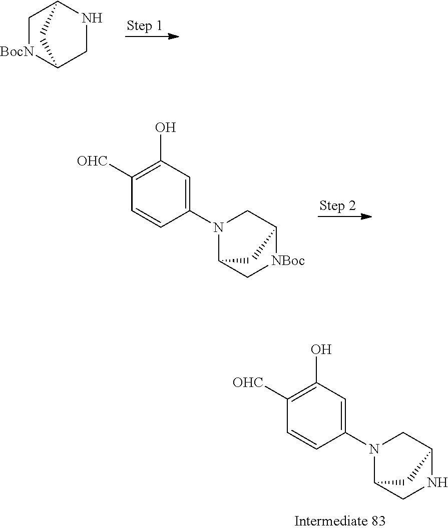 [SMILES: CC(C)(C)OC(=O)N1C[C@@H]2C[C@H]1CN2 ; CC(C)(C)OC(=O)N1C[C@@H]2C[C@H]1CN2c1ccc(C=O)c(O)c1 ; O=Cc1ccc(N2C[C@@H]3C[C@H]2CN3)cc1O]

Following the general procedures described in Intermediate 48, the reactions were carried out using corresponding starting materials. 4-((1S,4S)-2,5-diazabicyclo[2.2.1]heptan-2-yl)-2-hydroxybenzaldehyde (Intermediate 83, 300 mg, crude) was obtained as a black solid. 4-((1S,4S)-2,5-diazabicyclo[2.2.1]heptan-2-yl)-2-hydroxybenzaldehyde (Intermediate 83, 300 mg, crude) was obtained as a black solid. MS: m/z=219.1 [M+H]⁺.

### Intermediate 84: 4-((1R,4R)-2,5-Diazabicyclo[2.2.1]heptan-2-yl)-2-hydroxybenzaldehyde

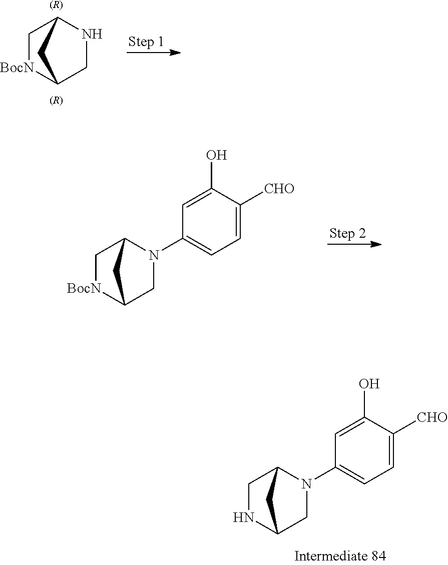

Following the general procedures described in Intermediate 48, the reactions were carried out using corresponding starting materials. 4-((1R,4R)-2,5-diazabicyclo[2.2.1]heptan-2-yl)-2-hydroxybenzaldehyde (Intermediate 84, 300 mg, crude) was obtained as a black solid and used directly in the next step. MS: m/z=219.2 [M+H]⁺.

### Intermediate 85: 3-(3-(4-((2,5-Diazabicyclo[2.2.2]octan-2-yl)methyl)phenyl)-5-phenyl-3H-imidazo[4,5-b]pyridin-2-yl)pyridin-2-amine

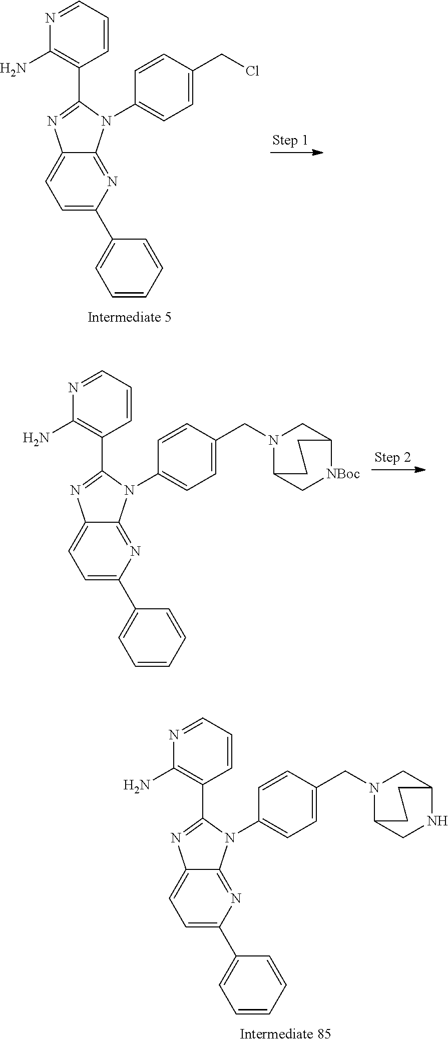 [SMILES: Nc1ncccc1-c1nc2ccc(-c3ccccc3)nc2n1-c1ccc(CCl)cc1 ; CC(C)(C)OC(=O)N1CC2CCC1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2CC3CCC2CN3)cc1]

Following the general procedures described in Intermediate 78, the reactions were carried out using corresponding starting materials. 3-(3-(4-((2,5-Diazabicyclo[2.2.2]octan-2-yl)methyl)phenyl)-5-phenyl-3H-imidazo[4,5-b]pyridin-2-yl)pyridin-2-amine (Intermediate 85, 44.9 mg, yield: 99%) as a yellow solid. MS: m/z=488.2 [M+H]⁺. ¹H NMR (400 MHz, Dimethylsulfoxide-d₆) δ 12.11-11.44 (m, 1H), 10.27-9.61 (m, 2H), 8.56-8.40 (m, 1H), 8.38 (d, J=8.4 Hz, 1H), 8.15 (dd, J=6.0, 1.2 Hz, 1H), 8.10-7.99 (m, 5H), 7.86 (dd, J=7.2, 1.2 Hz, 1H), 7.68 (d, J=8.4 Hz, 2H), 7.51-7.46 (m, 2H), 7.46-7.40 (m, 1H), 6.91 (dd, J=7.6, 6.4 Hz, 1H), 4.62 (br s, 2H), 4.01-3.79 (m, 4H), 3.76-3.70 (m, 2H), 2.21 (m, 2H), 1.97-1.80 (m, 2H).

### Intermediate 86: 4-(2,5-Diazabicyclo[2.2.2]octan-2-yl)-2-hydroxybenzaldehyde

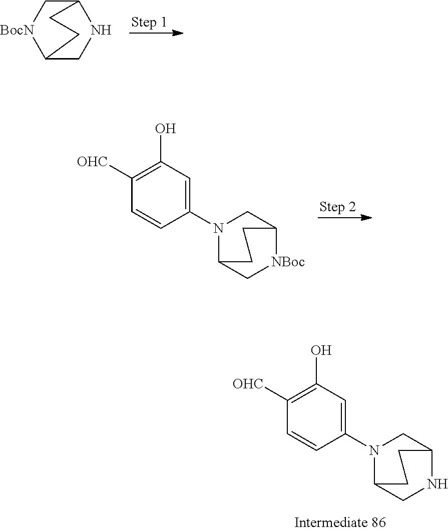 [SMILES: CC(C)(C)OC(=O)N1CC2CCC1CN2 ; CC(C)(C)OC(=O)N1CC2CCC1CN2c1ccc(C=O)c(O)c1 ; O=Cc1ccc(N2CC3CCC2CN3)cc1O]

Following the general procedures described in Intermediate 48, the reactions were carried out using corresponding starting materials. 4-(2,5-diazabicyclo[2.2.2]octan-2-yl)-2-hydroxybenzaldehyde (Intermediate 86, 300 mg, crude) was obtained as a black solid. MS: m/z=233.1 [M+H]⁺.

### Intermediate 87: 2-Methoxy-6)piperidin-4-yloxy)benzaldehyde

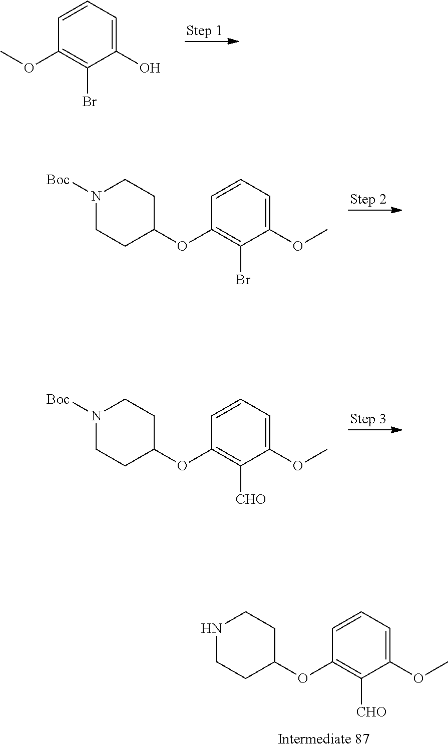

### Step 1: tert-Butyl 4-(2-bromo-3-methoxyphenoxy)piperidine-1-carboxylate

To a mixture of 2-bromo-3-methoxyphenol (1 g, 4.93 mmol), tert-butyl 4-hydroxypiperidine-1-carboxylate (991 mg, 4.93 mmol) and PPh₃ (1.55 g, 5.91 mmol) in THF (10 mL) was added DIAD (1.2 g, 5.91 mmol) at 0 °C The mixture was stirred at 20 °C for 16 hr. The reaction mixture was diluted with H₂O (50 mL) and extracted with CH₂Cl₂ (30 mL×3). The combined organic layers were washed with brine (30 mL), dried over Na₂SO₄, filtered and concentrated under reduced pressure. After purified by silica gel flash chromatography (Eluent of 0%~10% EtOAc in petroleum ether), tert-butyl 4-(2-bromo-3-methoxyphenoxy)piperidine-1-carboxylate (1.7 g, yield: 84%) was obtained as an off white solid. ¹H NMR (400 MHz, Chloroform-d) δ 7.22-7.12 (m, 1H), 6.59-6.55 (m, 2H), 4.62-4.53 (m, 1H), 3.90 (s, 3H), 3.67-3.58 (m, 2H), 3.54-3.46 (m, 2H), 1.89-1.83 (m, 4H), 1.47 (s, 9H).

### Step 2: tert-Butyl 4-(2-formyl-3-methoxyphenoxy)piperidine-1-carboxylate

To a mixture of tert-butyl 4-(2-bromo-3-methoxyphenoxy)piperidine-1-carboxylate (1 g, 2.59 mmol) in THF (5 mL) was added n-BuLi (2.5 M, 2.07 mL) at −78 °C The mixture was stirred at −78 °C for 0.5 hr under N₂. Then DMF (283 mg, 3.88 mmol) was added at −78 °C The mixture was stirred at −78 °C for 0.5 hr under N₂. The reaction mixture was diluted with NH₄C1 (30 mL) and extracted with EtOAc (50 mL×3). The combined organic layers were washed with brine (20 mL), dried over Na₂SO₄, filtered and concentrated under reduced pressure. After purified by silica gel flash chromatography (Eluent of 0%-30% EtOAc in petroleum ether), tert-butyl 4-(2-formyl-3-methoxyphenoxy)piperidine-1-carboxylate (600 mg, yield: 68%) was obtained as a light yellow oil. MS: m/z=358.1 [M+Na]. ¹H NMR (400 MHz, Chloroform-d) δ 10.53 (s, 1H), 7.44-7.39 (m, 1H), 6.56 (dd, J=8.4, 3.2 Hz, 2H), 4.65-4.58 (m, 1H), 3.90 (s, 3H), 3.64-3.58 (m, 2H), 3.49-3.42 (m, 2H), 1.93-1.87 (m, 2H), 1.86-1.79 (m, 2H), 1.46 (s, 9H).

### Step 3: 2-Methoxy-6-(piperidin-4-yloxy)benzaldehyde

To a mixture of tert-butyl 4-(2-formyl-3-methoxyphenoxy)piperidine-1-carboxylate (200 mg, 596 μmol) in CH₂Cl₂ (5 mL) was added HCl in 1,4-dioxane (4 M, 0.5 mL). The mixture was stirred at 20 °C for 2 hr. The reaction mixture concentrated under reduced pressure to give 2-methoxy-6-(piperidin-4-yloxy)benzaldehyde (Intermediate 87, 160 mg, HCl salt, yield: 99%) as an off white solid which was used directly for next step. MS: m/z=236.1 [M+H]⁺.

### Intermediate 88: 2-Methoxy-4-(piperidin-4-yloxy)benzaldehyde

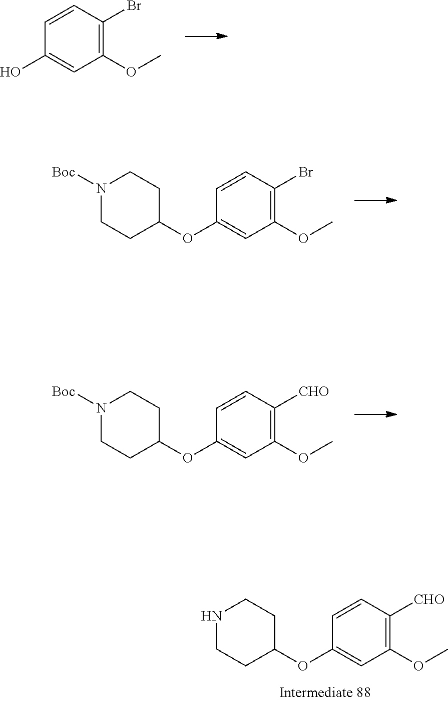

Following the general procedures described in Intermediate 87, the reactions were carried out using corresponding starting materials. 2-Methoxy-4-(piperidin-4-yloxy)benzaldehyde (Intermediate 88, 170 mg, HCl salt, yield: 92%) was obtained as a light yellow solid. MS: m/z=236.0 [M+H]⁺.

### Intermediate 89: 2-Methoxy-5-(piperidin-4-yloxy)benzaldehyde

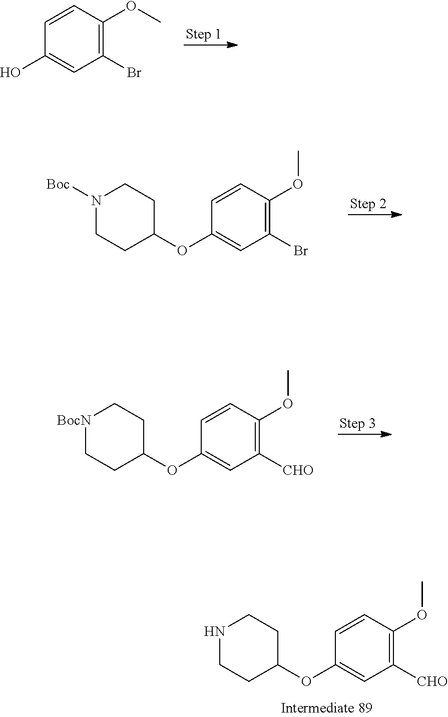

Following the general procedures described in Intermediate 87, the reactions were carried out using corresponding starting materials. 2-Methoxy-5-(piperidin-4-yloxy)benzaldehyde (Intermediate 89, 260 mg, HCl salt, yield: 78%) was obtained as a light yellow solid. MS: m/z=236.1 [M+H]⁺.

### Intermediate 90: 3-(3-(4-(((3aR,6aS)-Hexahydropyrrolo[3,4-c]pyrrol-2(1H)-yl)methyl)phenyl)-5-phenyl-3H-imidazo[4,5-b]pyridin-2-yl)pyridin-2-amine

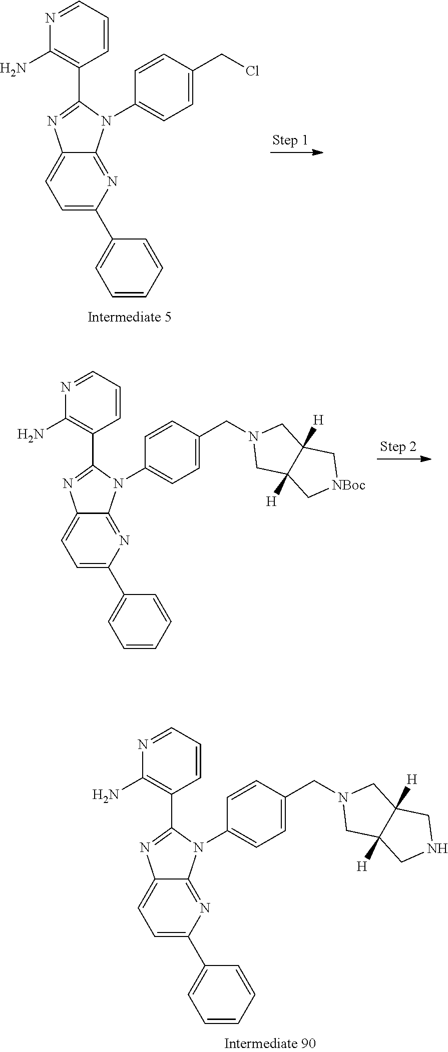 [SMILES: Nc1ncccc1-c1nc2ccc(-c3ccccc3)nc2n1-c1ccc(CCl)cc1 ; CC(C)(C)OC(=O)N1C[C@H]2CN(Cc3ccc(-n4c(-c5cccnc5N)nc5ccc(-c6ccccc6)nc54)cc3)C[C@H]2C1 ; Nc1ncccc1-c1nc2ccc(-c3ccccc3)nc2n1-c1ccc(CN2C[C@H]3CNC[C@H]3C2)cc1]

Following the general procedures described in Intermediate 37, the reactions were carried out using corresponding starting materials. 3-(3-(4-(((3aR,6aS)-Hexahydropyrrolo[3,4-c]pyrrol-2(1H)-yl)methyl)phenyl)-5-phenyl-3H-imidazo[4,5-b]pyridin-2-yl)pyridin-2-amine (Intermediate 90, 35.4 mg, yield: 85%) was obtained as an off-white solid. MS: m/z=488.2 [M+H]⁺. ¹H NMR (400 MHz, Dimethylsulfoxide-d₆) 8.27 (d, J=8.0 Hz, 1H), 8.04-7.97 (m, 4H), 7.49-7.44 (m, 5H), 7.43-7.37 (m, 2H), 7.16 (dd, J=7.6, 1.6 Hz, 1H), 7.04 (br s, 2H), 6.38 (dd, J=7.6, 4.8 Hz, 1H), 3.60 (s, 2H), 2.85-2.76 (m, 2H), 2.58-2.51 (m, 7H), 2.31-2.26 (m, 2H).

### Intermediate 91: 4-((3aR,6aS)-Hexahydropyrrolo[3,4-c]pyrrol-2(1H)-yl)-2-hydroxybenzaldehyde

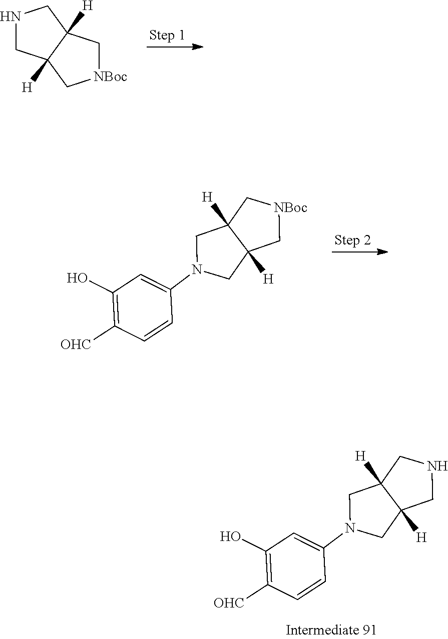 [SMILES: CC(C)(C)OC(=O)N1C[C@H]2CNC[C@H]2C1 ; CC(C)(C)OC(=O)N1C[C@@H]2CN(c3ccc(C=O)c(O)c3)C[C@@H]2C1 ; O=Cc1ccc(N2C[C@H]3CNC[C@H]3C2)cc1O]

Following the general procedures described in Intermediate 48, the reactions were carried out using corresponding starting materials. 4-((3aR,6aS)-Hexahydropyrrolo[3,4-c]pyrrol-2(1H)-yl)-2-hydroxybenzaldehyde (Intermediate 91, 280 mg,) was obtained as a brown solid. ¹H NMR (400 MHz, Methanol-d₄) δ 9.56 (s, 1H), 7.45-7.42 (m, 1H), 6.35 (d, J=8.8, 1H), 6.06 (s, 1H), 3.66-3.48 (m, 10H).

### Intermediate 92: 5-((3aR,6aS)-Hexahydropyrrolo[3,4-c]pyrrol-2(1H)-yl)-2-hydroxybenzaldehyde

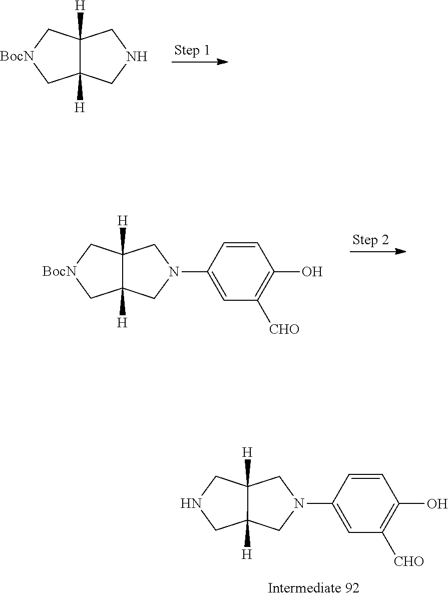 [SMILES: CC(C)(C)OC(=O)N1C[C@H]2CNC[C@H]2C1 ; CC(C)(C)OC(=O)N1C[C@@H]2CN(c3ccc(O)c(C=O)c3)C[C@@H]2C1 ; O=Cc1cc(N2C[C@H]3CNC[C@H]3C2)ccc1O]

Following the general procedures described in Intermediate 56, the reactions were carried out using corresponding starting materials. 5-((3aR,6aS)-Hexahydropyrrolo[3,4-c]pyrrol-2(1H)-yl)-2-hydroxybenzaldehyde (Intermediate 92, 72 mg, HCl salt) was obtained as a yellow solid. MS: m/z=232.9 [M+H]⁺.

### Intermediate 93: 3-(3-(4-(((3aR,6aR)-hexahydropyrrolo[3,4-c]pyrrol-2(1H)-yl)methyl)phenyl)-5-phenyl-3H-imidazo[4,5-b]pyridin-2-yl)pyridin-2-amine

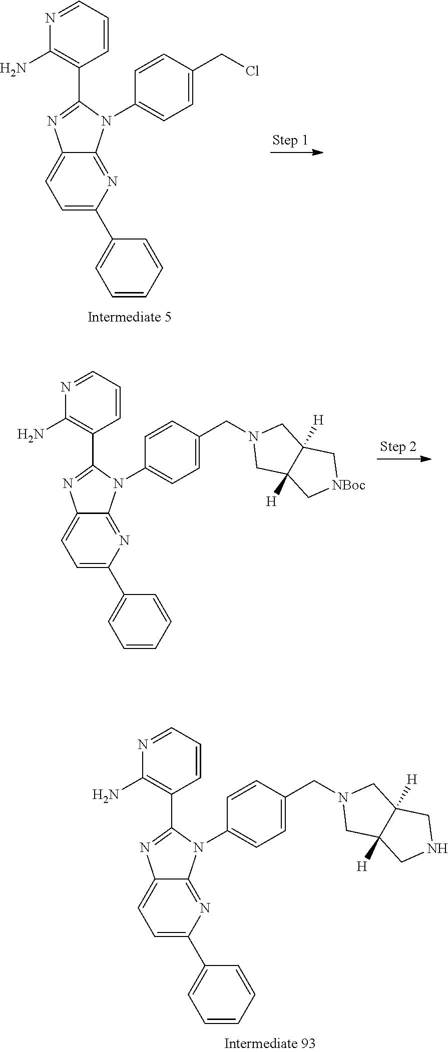 [SMILES: Nc1ncccc1-c1nc2ccc(-c3ccccc3)nc2n1-c1ccc(CCl)cc1 ; CC(C)(C)OC(=O)N1C[C@@H]2CN(Cc3ccc(-n4c(-c5cccnc5N)nc5ccc(-c6ccccc6)nc54)cc3)C[C@H]2C1 ; Nc1ncccc1-c1nc2ccc(-c3ccccc3)nc2n1-c1ccc(CN2C[C@H]3CNC[C@@H]3C2)cc1]

Following the general procedures described in Intermediate 37, the reactions were carried out using corresponding starting materials. 3-(3-(4-(((3aR,6aR)-Hexahydropyrrolo[3,4-c]pyrrol-2(1H)-yl)methyl)phenyl)-5-phenyl-3H-imidazo[4,5-b]pyridin-2-yl)pyridin-2-amine (Intermediate 93, 30 mg, HCl salt, yield: 33%) was obtained as a yellow solid. MS: m/z=488.3 [M+H]⁺. ¹H NMR (400 MHz, Dimethylsulfoxide-d₆) δ 12.14-11.98 (m, 1H), 9.92-9.78 (m, 1H), 9.73-9.54 (m, 1H), 8.37 (d, J=8.8 Hz, 1H), 8.33-8.19 (m, 1H), 8.13 (dd, J=6.0, 1.2 Hz, 1H), 8.09-8.04 (m, 3H), 7.86 (d, J=8.4 Hz, 2H), 7.81-7.76 (m, 1H), 7.66 (d, J=8.0 Hz, 2H), 7.51-7.41 (m, 3H), 6.89-6.83 (m, 1H), 4.66-4.51 (m, 2H), 3.72-3.63 (m, 2H), 3.20-3.10 (m, 2H), 3.04-2.93 (m, 2H), 2.85-2.76 (m, 1H), 2.64-2.57 (m, 1H), 2.46-2.33 (m, 2H).

### Intermediate 94: (S)-3-(3-(4-(1-Aminoethyl)phenyl)-5-phenyl-3H-imidazo[4,5-b]pyridin-2-yl)pyridin-2-amine

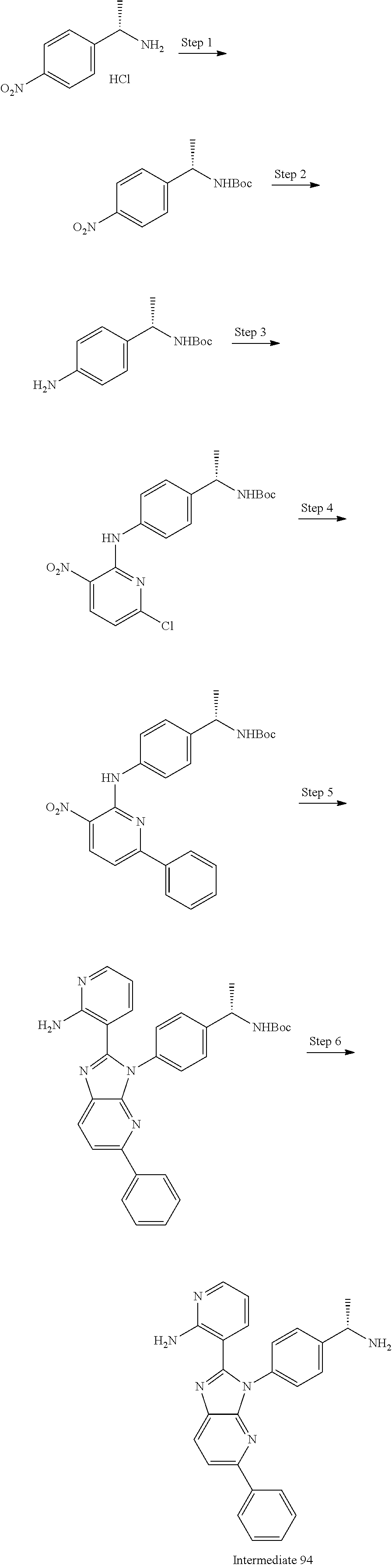

### Step 1: tert-Butyl (S)-(1-(4-nitrophenyl)ethyl)carbamate

A mixture of (S)-1-(4-nitrophenyl)ethan-1-amine (1.8 g, 10.8 mmol), Boc₂O (2.36 g, 10.8 mmol), NaHCO₃ (2.73 g, 32.5 mmol) in MeOH (100 mL) and H₂O (100 mL) was degassed and purged with N₂ three times. The mixture was stirred at 20 °C for 16 hr under N₂ atmosphere. The reaction mixture was concentrated under reduced pressure to remove MeOH. The residue was diluted with H₂O (50 mL) and extracted with EtOAc (100 mL×3). The combined organic layers were washed with brine (50 mL), dried over Na₂SO₄, filtered and concentrated under reduced pressure. tert-Butyl (S)-(1-(4-nitrophenyl)ethyl)carbamate (2.2 g, yield: 76%) was obtained as a light yellow oil which was used directly. MS: m/z=211.3 [M-55]⁺.

### Step 2: tert-Butyl (S)-(1-(4-aminophenyl)ethyl)carbamate

A mixture of tert-butyl (S)-(1-(4-nitrophenyl)ethyl)carbamate (2 g, 7.51 mmol), Fe (2.10 g, 37.5 mmol) and NH₄Cl (4.05 g, 75.1 mmol) in EtOH (12 mL) and H₂O (4 mL) was degassed and purged with N₂ three times. The mixture was stirred at 85 °C for 2 hr under N₂ atmosphere. The reaction mixture was filtered and concentrated under reduced pressure to remove solvent, tert-butyl (S)-(1-(4-aminophenyl)ethyl)carbamate (1.78 g, yield: 98%) was obtained as a light yellow oil which was used directly for next step. MS: m/z=259.1 [M+Na]⁺.

### Step 3: tert-Butyl (S)-(1-(4-((6-chloro-3-nitropyridin-2-yl)amino)phenyl)ethyl)carbamate

A mixture of 2,6-dichloro-3-nitropyridine (969 mg, 5.02 mmol), tert-butyl (S)-(1-(4-aminophenyl)ethyl)carbamate (1.78 g, 7.53 mmol) and DIEA (1.95 g, 15.1 mmol) in 1,4-dioxane (20 mL) was degassed and purged with N₂ three times. The mixture was stirred at 80 °C for 16 hr under N₂ atmosphere. The reaction mixture was concentrated under reduced pressure. After purified by silica gel flash chromatography (Eluent of 0%~1% MeOH in CH₂Cl₂), tert-butyl (S)-(1-(4-((6-chloro-3-nitropyridin-2-yl)amino)phenyl)ethyl)carbamate (1 g, yield: 49%) was obtained as an orange solid. MS: m/z=415.2 [M+Na]⁺.

### Step 4: tert-Butyl (S)-(1-(4-((3-nitro-6-phenylpyridin-2-yl)amino)phenyl)ethyl)carbamate

A mixture of tert-butyl (S)-(1-(4-((6-chloro-3-nitropyridin-2-yl)amino)phenyl)ethyl)carbamate (6.1 g, 14.0 mmol), phenylboronic acid (2.04 g, 16.8 mmol), Pd(dppf)Cl₂ (1.02 g, 1.40 mmol), Cs₂CO₃ (13.7 g, 42.0 mmol) in 1,4-dioxane (100 mL) and H₂O (10 mL) was degassed and purged with N₂ three times. The mixture was stirred at 100 °C for 16 hr under N₂ atmosphere. After cooling to room temperature, the reaction mixture was diluted with CH₂Cl₂ (100 mL). The organic layer was washed with H₂O (50 mL) and brine (300 mL), dried over Na₂SO₄, filtered and concentrated under reduced pressure. After purified by silica gel flash chromatography (Eluent of 0%-1% MeOH in CH₂Cl₂), tert-butyl (S)-(1-(4-((3-nitro-6-phenylpyridin-2-yl)amino)phenyl)ethyl)carbamate (4.34 g, yield: 70%) was obtained as an orange solid. MS: m/z=435.3 [M+H]+. ¹H NMR (400 MHz, Chloroform-d) δ 10.30 (s, 1H), 8.58 (d, J=8.4 Hz, 1H), 8.09-8.02 (m, 2H), 7.73 (d, J=8.4 Hz, 2H), 7.52-7.48 (m, 3H), 7.36 (d, J=8.4 Hz, 2H), 7.30 (d, J=8.8 Hz, 1H), 4.91-4.68 (m, 2H), 1.49 (d, J=5.6 Hz, 3H), 1.44 (s, 9H).

### Step 5: tert-Butyl (S)-(1-(4-(2-(2-aminopyridin-3-yl)-5-phenyl-3H-imidazo[4,5-b]pyridin-3-yl)phenyl)ethyl)carbamate

To a solution of tert-butyl (S)-(1-(4-((3-nitro-6-phenylpyridin-2-yl)amino)phenyl)ethyl)carbamate (4.34 g, 9.99 mmol) and 2-aminonicotinaldehyde (1.46 g, 11.9 mmol) in DMSO (40 mL) was added Na₂S₂O₄ (3.48 g, 19.9 mmol). The mixture was stirred at 100 °C for 16 hr. After cooling to room temperature, the reaction mixture was diluted with CH₂Cl₂ (100 mL). The organic layer was washed with H₂O (50 mL) and brine (20 mL), dried over Na₂SO₄, filtered and concentrated under reduced pressure. The residue (4.1 g) was dissolved in CH₂Cl₂ (10 mL). Boc₂O (3.30 g, 15.1 mmol) and TEA (2.04 g, 20.1 mmol) were added. The mixture was stirred at 25 °C for 1 hr. The reaction mixture was diluted with H₂O (50 mL) and extracted with CH₂Cl₂ (50 mL×3). The combined organic layers dried over Na₂SO₄, filtered and concentrated under reduced pressure. After purified by silica gel flash chromatography (Eluent of 0%~5% MeOH in CH₂Cl₂), tert-butyl (S)-(1-(4-(2-(2-aminopyridin-3-yl)-5-phenyl-3H-imidazo[4,5-b]pyridin-3-yl)phenyl)ethyl)carbamate (1.7 g, yield: 29%) was obtained as a yellow solid. MS: m/z=507.2 [M+H]⁺. ¹H NMR (400 MHz, Chloroform-d) δ 8.12 (d, J=8.4 Hz, 1H), 8.06 (dd, J=6.8, 1.6 Hz, 1H), 8.02 (d, J=7.6 Hz, 2H), 7.80 (d, J=8.0 Hz, 1H), 7.47-7.40 (m, 7H), 7.12-7.09 (m, 1H), 6.6 (br s, 2H), 6.37 (dd, J=7.6, 5.2 Hz, 1H), 4.96-4.76 (m, 2H), 1.53 (d, J=6.0 Hz, 3H), 1.45 (s, 9H).

### Step 6: (S)-3-(3-(4-(1-Aminoethyl)phenyl)-5-phenyl-3H-imidazo[4,5-b]pyridin-2-yl)pyridin-2-amine

To a solution of tert-butyl (S)-(1-(4-(2-(2-aminopyridin-3-yl)-5-phenyl-3H-imidazo[4,5-b]pyridin-3-yl)phenyl)ethyl)carbamate (1.3 g, 2.57 mmol) in CH₂Cl₂ (10 mL) was added HCl in 1,4-dioxane (4 M, 10.2 mL). The mixture was stirred at 25 °C for 0.5 hr. The reaction mixture was concentrated under reduced pressure to give (S)-3-(3-(4-(1-aminoethyl)phenyl)-5-phenyl-3H-imidazo[4,5-b]pyridin-2-yl)pyridin-2-amine (Intermediate 94, 1.2 g, HCl salt, yield: 83%) as a yellow solid. MS: m/z=407.1 [M+H]⁺. ¹H NMR (400 MHz, Methanol-d₄) δ 8.14 (d, J=8.4 Hz, 1H), 8.02-7.98 (m, 2H), 7.96 (dd, J=4.8, 1.6 Hz, 1H), 7.89 (d, J=8.4 Hz, 1H), 7.53 (d, J=8.4 Hz, 2H), 7.42-7.34 (m, 5H), 7.27 (dd, J=7.6, 1.6 Hz, 1H), 6.44 (dd, J=7.6, 5.2 Hz, 1H), 4.19-4.08 (m, 1H), 1.45 (d, J=6.8 Hz, 3H).

### Intermediate 95: 3-(5-(2-Fluorophenyl)-3-(4-(piperazin-1-ylmethyl)phenyl)-3H-imidazo[4,5-b]pyridin-2-yl)pyridin-2-amine hydrochloride

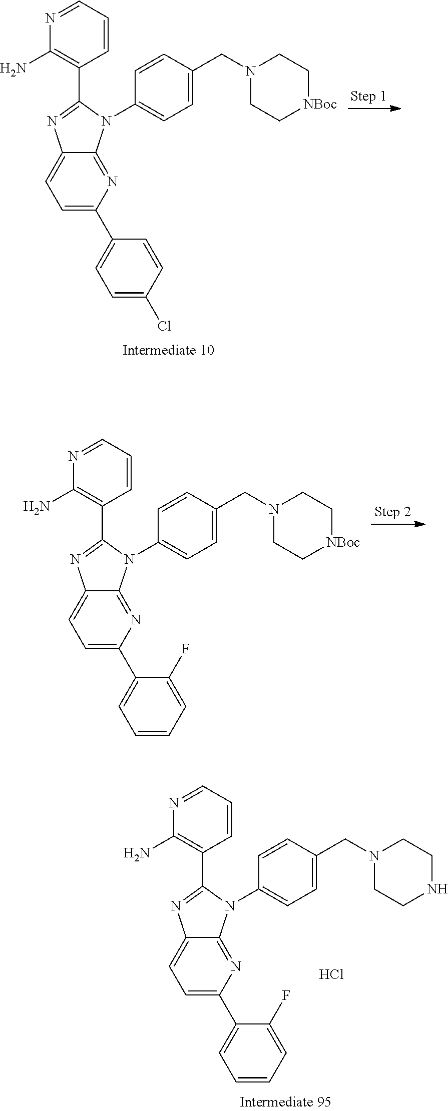 [SMILES: CC(C)(C)OC(=O)N1CCN(Cc2ccc(-n3c(-c4cccnc4N)nc4ccc(-c5ccc(Cl)cc5)nc43)cc2)CC1 ; CC(C)(C)OC(=O)N1CCN(Cc2ccc(-n3c(-c4cccnc4N)nc4ccc(-c5ccccc5F)nc43)cc2)CC1 ; Nc1ncccc1-c1nc2ccc(-c3ccccc3F)nc2n1-c1ccc(CN2CCNCC2)cc1 ; Cl]

### Step 1: tert-Butyl 4-(4-(2-(2-aminopyridin-3-yl)-5-(2-fluorophenyl)-3H-imidazo[4,5-b]pyridin-3-yl)benzyl)piperazine-1-carboxylate

To a solution of Intermediate 10 (500 mg, 961 μmol) and (2-fluorophenyl)boronic acid (134 mg, 961 μmol) in 1,4-dioxane (10 mL) and H₂O (2 mL) were added Cs₂CO₃ (940 mg, 2.88 mmol) and Pd(dppf)Cl₂ (70.4 mg, 96.1 μmol). The mixture was degassed and purged with N₂ three times and stirred at 80 °C for 16 hr under N₂ atmosphere. Then the reaction mixture was diluted with H₂O (50 mL) and extracted with CH₂Cl₂ (50 mL×2). The combined organic layers were dried over Na₂SO₄, filtered and concentrated under reduced pressure. The crude product was purified by silica gel flash chromatography (Eluent of 30~80% EtOAc in petroleum ether) to give tert-Butyl 4-(4-(2-(2-aminopyridin-3-yl)-5-(2-fluorophenyl)-3H-imidazo[4,5-b]pyridin-3-yl)benzyl)piperazine-1-carboxylate (500 mg, yield: 87%) as a yellow solid. MS: m/z=580.2 [M+H]⁺. ¹H NMR (400 MHz, Dimethylsulfoxide-d₆) δ 8.30 (d, J=8.4 Hz, 1H), 8.00 (dd, J=4.8, 1.6 Hz, 1H), 7.90-7.70 (m, 2H), 7.56-7.40 (m, 5H), 7.37-7.26 (m, 2H), 7.16 (dd, J=8.0, 2.0 Hz, 1H), 6.99 (br s, 2H), 6.39 (dd, J=7.6, 4.8 Hz, 1H), 3.57 (s, 2H), 3.36-3.33 (m, 4H), 2.42-2.30 (m, 4H), 1.40 (s, 9H).

### Step 2: 3-(5-(2-Fluorophenyl)-3-(4-(piperazin-1-ylmethyl)phenyl)-3H-imidazo[4,5-b]pyridin-2-yl)pyridin-2-amine hydrochloride

To a solution of tert-butyl 4-(4-(2-(2-aminopyridin-3-yl)-5-(2-fluorophenyl)-3H-imidazo[4,5-b]pyridin-3-yl)benzyl)piperazine-1-carboxylate (500 mg, 863 μmol) in 1,4-dioxane (5 mL) was added HCl/1,4-dioxane (4M, 5 mL). The mixture was stirred at 25 °C for 0.5 hr.

The reaction mixture was filtered and concentrated under reduced pressure to give 3-(5-(2-fluorophenyl)-3-(4-(piperazin-1-ylmethyl)phenyl)-3H-imidazo[4,5-b]pyridin-2-yl)pyridin-2-amine (Intermediate 95, 400 mg, yield: 92%, HCl salt) as a yellow solid. MS: m/z=480.2 [M+H]+. ¹H NMR (400 MHz, Dimethylsulfoxide-d₆) δ 12.82-11.99 (m, 1H), 10.20-9.62 (m, 2H), 8.53-8.43 (m, 1H), 8.40 (d, J=8.0 Hz, 1H), 8.15 (dd, J=6.0, 1.6 Hz, 1H), 7.91-7.81 (m, 5H), 7.67 (d, J=8.4 Hz, 2H), 7.52-7.46 (m, 1H), 7.39-7.30 (m, 2H), 6.98 (dd, J=7.6, 6.4 Hz, 1H), 4.48 (s, 2H), 3.53-3.48 (m, 4H), 2.50 (m, 4H). ¹⁹F NMR (400 MHz, Dimethylsulfoxide-d₆) δ −117.18.

### Intermediate 96: 3-(5-(4-fluorophenyl)-3-(4-(piperazin-1-ylmethyl)phenyl)-3H-imidazo[4,5-b]pyridin-2-yl)pyridin-2-amine hydrochloride

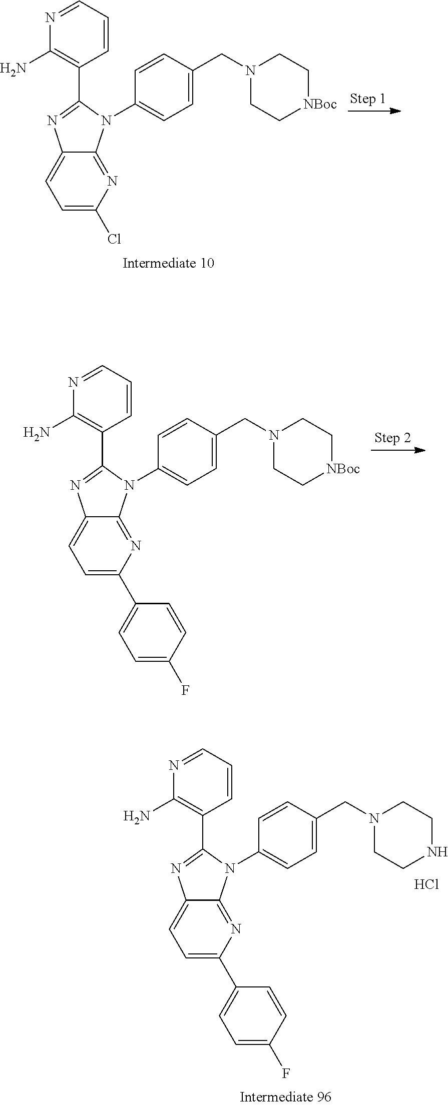 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5ccc(F)cc5)nc43)cc2)CC1 ; Nc1ncccc1-c1nc2ccc(-c3ccc(F)cc3)nc2n1-c1ccc(CN2CCNCC2)cc1 ; Cl]

### Step 1: tert-Butyl 4-(4-(2-(2-aminopyridin-3-yl)-5-(4-fluorophenyl)-3H-imidazo[4,5-b]pyridin-3-yl)benzyl)piperazine-1-carboxylate

To a solution of Intermediate 10 (500 mg, 961 μmol) and (4-fluorophenyl)boronic acid (135 mg, 961 μmol) in 1,4-dioxane (10 mL) and H₂O (2 mL) were added Cs₂CO₃ (940 mg, 2.88 mmol) and Pd(dppf)Cl₂ (70.4 mg, 96.1 μmol). The mixture was degassed and purged with N₂ three times and stirred at 80 °C for 16 hr under N₂ atmosphere. Then the reaction mixture was diluted with H₂O (50 mL) and extracted with CH₂Cl₂ (50 mL×2). The combined organic layers were dried over Na₂SO₄, filtered and concentrated under reduced pressure. The crude product was purified by silica gel flash chromatography (Eluent of 50-100% EtOAc in petroleum ether) to give tert-butyl 4-(4-(2-(2-aminopyridin-3-yl)-5-(4-fluorophenyl)-3H-imidazo[4,5-b]pyridin-3-yl)benzyl)piperazine-1-carboxylate (500 mg, yield: 86%) as a yellow solid. MS: m/z=580.3 [M+H]⁺. ¹H NMR (400 MHz, Dimethylsulfoxide-d₆) δ 8.26 (d, J=8.4 Hz, 1H), 8.13-8.04 (m, 2H), 8.01-7.87 (m, 2H), 7.52-7.41 (m, 4H), 7.29 (t, J=8.8 Hz, 2H), 7.14 (dd, J=7.6, 1.6 Hz, 1H), 7.03 (s, 2H), 6.38 (dd, J=8.0, 4.8 Hz, 1H), 3.59 (s, 2H), 3.37-3.33 (m, 4H), 2.40-2.26 (m, 4H), 1.40 (s, 9H). ¹⁹F NMR (400 MHz, Dimethylsulfoxide-d₆) δ −113.59.

### Step 2: 3-(5-(4-Fluorophenyl)-3-(4-(piperazin-1-ylmethyl)phenyl)-3H-imidazo[4,5-b]pyridin-2-yl)pyridin-2-amine hydrochloride

To a solution of tert-butyl 4-(4-(2-(2-aminopyridin-3-yl)-5-(4-fluorophenyl)-3H-imidazo[4,5-b]pyridin-3-yl)benzyl)piperazine-1-carboxylate (500 mg, 863 μmol) in 1,4-dioxane (5 mL) was added HCl/1,4-dioxane (4 M, 5 mL). The mixture was stirred at 25 °C for 0.5 hr.

The reaction mixture was filtered and concentrated under reduced pressure to give 3-(5-(4-fluorophenyl)-3-(4-(piperazin-1-ylmethyl)phenyl)-3H-imidazo[4,5-b]pyridin-2-yl)pyridin-2-amine (Intermediate 96, 400 mg HCl salt, yield: 92%) as a yellow solid. MS: m/z=480.2 [M+H]⁺. ¹H NMR (400 MHz, Dimethylsulfoxide-d₆) δ 13.00-12.05 (m, 1H), 10.26-9.46 (m, 2H), 8.60-8.48 (m, 1H), 8.38 (d, J=8.4 Hz, 1H), 8.19-8.06 (m, 4H), 7.90-7.82 (m, 3H), 7.68 (d, J=8.4 Hz, 2H), 7.32 (t, J=9.2 Hz, 2H), 7.03-6.93 (m, 1H), 4.51 (br s, 2H), 3.55-3.50 (m, 4H), 2.59-2.51 (m, 4H). ¹⁹F NMR (400 MHz, Dimethylsulfoxide-d₆) δ −113.04.

### Intermediate 97: 3-(3-(4-(Chloromethyl)phenyl)-5-(2-chlorophenyl)-3H-imidazo[4,5-b]pyridin-2-yl)pyridin-2-amine

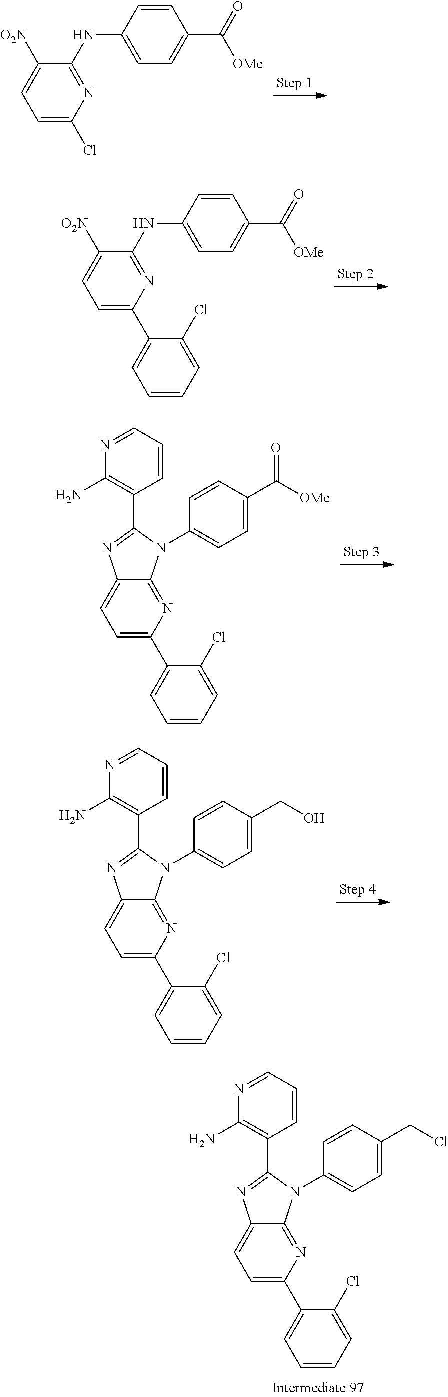

### Step 1: Methyl 4-((6-(2-chlorophenyl)-3-nitropyridin-2-yl)amino)benzoate

To a mixture of methyl 4-((6-chloro-3-nitropyridin-2-yl)amino)benzoate (refer to Intermediate 5 for detail procedures, 5 g, 16.2 mmol), (2-chlorophenyl)boronic acid (3.3 g, 21.1 mmol), Pd(dppf)Cl₂ (1.19 g, 1.63 mmol), Cs₂CO₃ (15.8 g, 48.7 mmol) in dioxane (50 mL) and H₂O (5 mL) was degassed and purged with N₂ three times. The mixture was stirred at 100 °C for 12 hr under N₂ atmosphere. The reaction mixture was diluted with water (20 mL) and extracted with CH₂Cl₂ (30 mL×2). The combined organic layers were dried over Na₂SO₄, filtered and concentrated under reduced pressure. The residue was purified by silica gel flash chromatography (Eluent of 0~10% MeOH in CH₂Cl₂) to give methyl 4-((6-(2-chlorophenyl)-3-nitropyridin-2-yl)amino)benzoate (1.5 g, yield: 21%) as a yellow solid. MS: m/z=384.0 [M+H]+. ¹H NMR (400 MHz, Chloroform-d) δ 10.49 (br s, 1H), 8.63 (d, J=8.8 Hz, 1H), 8.04 (d, J=6.8 Hz, 2H), 7.90 (d, J=9.2 Hz, 2H), 7.68-7.63 (m, 1H), 7.56-7.52 (m, 1H), 7.46-7.39 (m, 2H), 7.33 (d, J=8.8 Hz, 1H), 3.91 (s, 3H).

### Step 2: Methyl 4-(2-(2-aminopyridin-3-yl)-5-(2-chlorophenyl)-3H-imidazo[4,5-b]pyridin-3-yl)benzoate

To a solution of methyl 4-((6-(2-chlorophenyl)-3-nitropyridin-2-yl)amino)benzoate (1.5 g, 3.91 mmol) and 2-aminonicotinaldehyde (525 mg, 4.30 mmol) in DMSO (45 mL) was added Na₂S₂O₄ (3.20 g, 15.6 mmol). The mixture was stirred at 100 °C for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0-100% EtOAc in petroleum ether) to give methyl 4-(2-(2-aminopyridin-3-yl)-5-(2-chlorophenyl)-3H-imidazo[4,5-b]pyridin-3-yl) benzoate (800 mg, yield: 41%) as a yellow solid. MS: m/z=456.0 [M+H]⁺. ¹H NMR (400 MHz, Chloroform-d) δ 8.23-8.15 (m, 3H), 8.07 (dd, J=4.8, 1.6 Hz, 1H), 7.73 (d, J=8.4 Hz, 1H), 7.60-7.52 (m, 3H), 7.50-7.46 (m, 1H), 7.36-7.31 (m, 2H), 7.10 (dd, J=7.6, 1.6 Hz, 1H), 6.92 (br s, 2H), 6.43 (dd, J=8.0, 5.2 Hz, 1H), 3.96 (s, 3H).

### Step 3: (4-(2-(2-Aminopyridin-3-yl)-5-(2-chlorophenyl)-3H-imidazo[4,5-b]pyridin-3-yl)phenyl)methanol

To a solution of methyl 4-(2-(2-aminopyridin-3-yl)-5-(2-chlorophenyl)-3H-imidazo[4,5-b]pyridin-3-yl)benzoate (800 mg, 1.75 mmol) in THF (50 mL) was added LiAlH₄ (2.5 M, 1.05 mL) at 0 °C The mixture was stirred at 25 °C for 2 hr. The reaction mixture was quenched with Na₂SO₄·10H₂O (120 mg) at 0 °C and filtered. The filtrate was concentrated under reduced pressure to give (4-(2-(2-aminopyridin-3-yl)-5-(2-chlorophenyl)-3H-imidazo[4,5-b]pyridin-3-yl)phenyl)methanol (700 mg, crude as a yellow solid.

### Step 4: 3-(3-(4-(Chloromethyl)phenyl)-5-(2-chlorophenyl)-3H-imidazo[4,5-b]pyridin-2-yl)pyridin-2-amine

To a solution of (4-(2-(2-aminopyridin-3-yl)-5-(2-chlorophenyl)-3H-imidazo[4,5-b]pyridin-3-yl)phenyl)methanol (700 mg, 1.64 mmol) in CH₂Cl₂ (10 mL) was added SOCl₂ (1.17 g, 9.82 mmol). The mixture was stirred at 40 °C for 2 hr. The mixture was concentrated under reduced pressure. 3-(3-(4-(Chloromethyl)phenyl)-5-(2-chlorophenyl)-3H-imidazo[4,5-b]pyridin-2-yl)pyridin-2-amine (Intermediate 97, 750 mg HCl salt) was obtained as a yellow solid. MS: m/z=446.0 [M+H]+.

### Intermediate 98: 3-(3-(4-(Chloromethyl)phenyl)-5-(4-chlorophenyl)-3H-imidazo[4,5-b]pyridin-2-yl)pyridin-2-amine

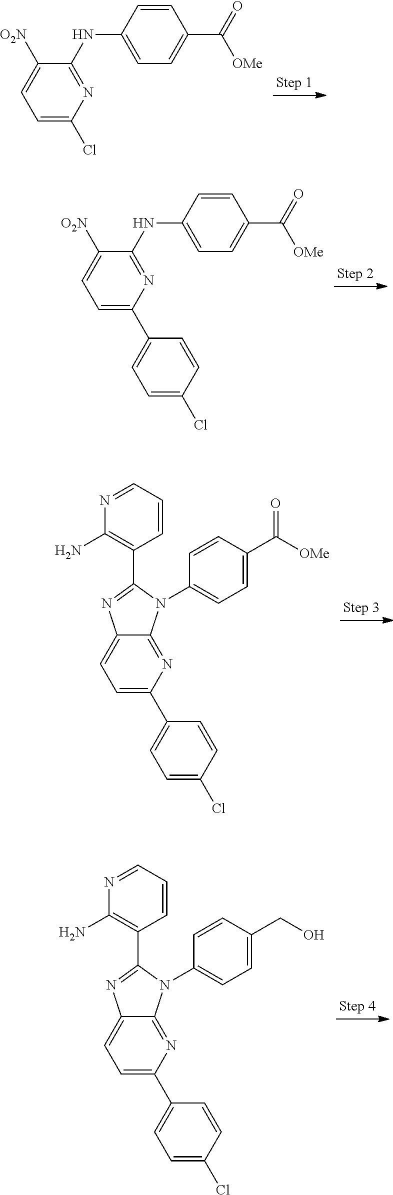 [SMILES: COC(=O)c1ccc(Nc2nc(Cl)ccc2[N+](=O)[O-])cc1 ; COC(=O)c1ccc(Nc2nc(-c3ccc(Cl)cc3)ccc2[N+](=O)[O-])cc1 ; COC(=O)c1ccc(-n2c(-c3cccnc3N)nc3ccc(-c4ccc(Cl)cc4)nc32)cc1 ; Nc1ncccc1-c1nc2ccc(-c3ccc(Cl)cc3)nc2n1-c1ccc(CO)cc1]

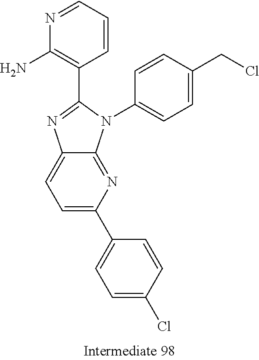 [SMILES: Nc1ncccc1-c1nc2ccc(-c3ccc(Cl)cc3)nc2n1-c1ccc(CCl)cc1]

### Step 1: Methyl 4-((6-(4-chlorophenyl)-3-nitropyridin-2-yl)amino)benzoate

A mixture of methyl 4-((6-chloro-3-nitropyridin-2-yl)amino)benzoate (refer to Intermediate 5 for detail procedures, 5 g, 16.3 mmol), (4-chlorophenyl)boronic acid (2.54 g, 16.3 mmol), Cs₂CO₃ (15.9 g, 48.8 mmol), and Pd(dppf)Cl₂ (2.38 g, 3.25 mmol) in 1,4-dioxane (100 mL) and H₂O (20 mL) was degassed and purged with N₂ three times. The mixture was stirred at 80 °C for 16 hr under N₂ atmosphere. The reaction mixture was diluted with H₂O (100 mL) and extracted with CH₂Cl₂ (50 mL×4). The combined organic layers were washed with brine (30 mL×3), dried over Na₂SO₄, filtered and concentrated under reduced pressure. The residue was purified by silica gel flash chromatography (Eluent of 35-45% EtOAc in petroleum ether) to give methyl 4-((6-(4-chlorophenyl)-3-nitropyridin-2-yl)amino)benzoate (5 g, yield: 80%) as a yellow solid. MS: m/z=383.9 [M+H]⁺.

### Step 2: Methyl 4-(2-(2-aminopyridin-3-yl)-5-(4-chlorophenyl)-3H-imidazo[4,5-b]pyridin-3-yl)benzoate

To a solution of methyl 4-((6-(4-chlorophenyl)-3-nitropyridin-2-yl)amino)benzoate (5 g, 13.0 mmol) and 2-aminonicotinaldehyde (1.75 g, 14.3 mmol) in DMSO (150 mL) was added Na₂S₂O₄ (9.07 g, 52.1 mmol). The mixture was stirred at 100 °C for 16 hr. The reaction mixture was diluted with H₂O (100 mL) and extracted with CH₂Cl₂ (500 mL×6). The combined organic layers were washed with brine (250 mL×2), dried over Na₂SO₄, filtered and concentrated under reduced pressure. The residue was purified by silica gel flash chromatography (Eluent of 25-30% EtOAc in CH₂Cl₂) to give methyl 4-(2-(2-aminopyridin-3-yl)-5-(4-chlorophenyl)-3H-imidazo[4,5-b]pyridin-3-yl)benzoate (2.3 g, yield: 34%) as a yellow solid. MS: m/z=456.0 [M+H]⁺.

### Step 3: (4-(2-(2-Aminopyridin-3-yl)-5-(4-chlorophenyl)-3H-imidazo[4,5-b]pyridin-3-yl)phenyl)methanol

To a solution of 4-(2-(2-aminopyridin-3-yl)-5-(4-chlorophenyl)-3H-imidazo[4,5-b]pyridin-3-yl)benzoate (2.3 g, 5.05 mmol) in THF (30 mL) was added LiAlH₄ (2.5 M, 2.42 mL) at 0 °C The mixture was stirred at 25 °C for 2 hr. The reaction mixture was quenched with Na₂SO₄·H₂O (4 g) at 0 °C, and the mixture was filtered, the filter cake was washed by CH₂C12 (30 mL×3). The filtrate was concentrated under reduced pressure to give (4-(2-(2-aminopyridin-3-yl)-5-(4-chlorophenyl)-3H-imidazo[4,5-b]pyridin-3-yl)phenyl)methanol (2.1 g, yield: 97%) as a yellow solid, which was directly used in the next step without purification. MS: m/z=428.1 [M+H]⁺.

### Step 4: 3-(3-(4-(Chloromethyl)phenyl)-5-(4-chlorophenyl)-3H-imidazo[4,5-b]pyridin-2-yl)pyridin-2-amine

To a solution of (4-(2-(2-aminopyridin-3-yl)-5-(4-chlorophenyl)-3H-imidazo[4,5-b]pyridin-3-yl)phenyl)methanol (1.85 g, 4.32 mmol) in CH₂Cl₂ (40 mL) was added SOCl₂ (1.54 g, 13.0 mmol) at 0 °C The mixture was stirred at 40 °C for 3 hr. The reaction mixture was quenched with H₂O (5 mL) at 0 °C, filtered and concentrated under reduced pressure to give 3-(3-(4-(chloromethyl)phenyl)-5-(4-chlorophenyl)-3H-imidazo[4,5-b]pyridin-2-yl)pyridin-2-amine (Intermediate 98, 1.9 g, yield: 98%) as a black brown solid. MS: m/z=455.9, 447.8 [M+H]⁺.

### Intermediate 99: 3-(5-(4-Chlorophenyl)-3-(4-(piperazin-1-ylmethyl)phenyl)-3H-imidazo[4,5-b]pyridin-2-yl)pyridin-2-amine

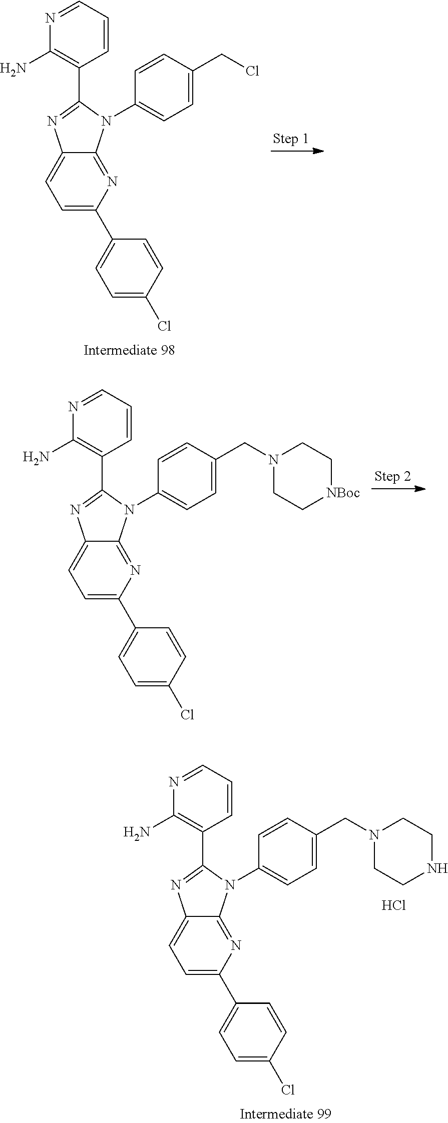 [SMILES: Nc1ncccc1-c1nc2ccc(-c3ccc(Cl)cc3)nc2n1-c1ccc(CCl)cc1 ; CC(C)(C)OC(=O)N1CCN(Cc2ccc(-n3c(-c4cccnc4N)nc4ccc(-c5ccc(Cl)cc5)nc43)cc2)CC1 ; Nc1ncccc1-c1nc2ccc(-c3ccc(Cl)cc3)nc2n1-c1ccc(CN2CCNCC2)cc1 ; Cl]

### Step 1: tert-Butyl 4-(4-(2-(2-aminopyridin-3-yl)-5-(4-chlorophenyl)-3H-imidazo[4,5-b]pyridin-3-yl)benzyl)piperazine-1-carboxylate

To a solution of Intermediate 98 (1 g, 2.24 mmol), tert-butyl piperazine-1-carboxylate (417 mg, 2.24 mmol) in DMF (10 mL) were added NaI (67.2 mg, 448 mol) and K₂CO₃ (929 mg, 6.72 mmol). The mixture was stirred at 25 °C for 16 hr. The residue was diluted with H₂O (30 mL) and extracted with EtOAc (30 mL×3). The combined organic layers were washed with brine (20 mL×2), dried over Na₂SO₄, filtered and concentrated under reduced pressure. The residue was purified by silica gel flash chromatography (Eluent of 0-4% MeOH in CH₂Cl₂) to give tert-butyl 4-(4-(2-(2-aminopyridin-3-yl)-5-(4-chlorophenyl)-3H-imidazo[4,5-b]pyridin-3-yl)benzyl)piperazine-1-carboxylate (550 mg, yield: 40%) as a yellow solid. MS: m/z=596.1 [M+H]⁺.

### Step 2: 3-(5-(4-Chlorophenyl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(4-chlorophenyl)-3H-imidazo[4,5-b]pyridin-3-yl)benzyl)piperazine-1-carboxylate (550 mg, 923 μmol) in 1,4-dioxane (5 mL) was added 4.0 M HCl in 1,4-dioxane (3 mL) at 25 °C The mixture was stirred at 25 °C for 1 hr. The reaction was concentrated under reduced pressure to give 3-(5-(4-chlorophenyl)-3-(4-(piperazin-1-ylmethyl)phenyl)-3H-imidazo[4,5-b]pyridin-2-yl)pyridin-2-amine (Intermediate 99, 550 mg, HCl salt, yield: 98%) as a yellow solid. MS: m/z=496.0 [M+H]+. ¹H NMR (400 MHz, Methanol-4) δ 8.33 (d, J=8.4 Hz, 1H), 8.08-8.02 (m, 4H), 7.95 (d, J=8.4 Hz, 2H), 7.90 (d, J=7.6 Hz, 1H), 7.74 (d, J=8.0 Hz, 2H), 7.45 (d, J=8.8 Hz, 2H), 6.94 (t, J=7.2 Hz, 1H), 4.67 (s, 2H), 3.80-3.64 (m, 8H).

### Intermediate 100: 3-(5-(Cyclohex-1-en-1-yl)-3-(4-(piperazin-1-ylmethyl)phenyl)-3H-imidazo[4,5-b]pyridin-2-yl)pyridin-2-amine

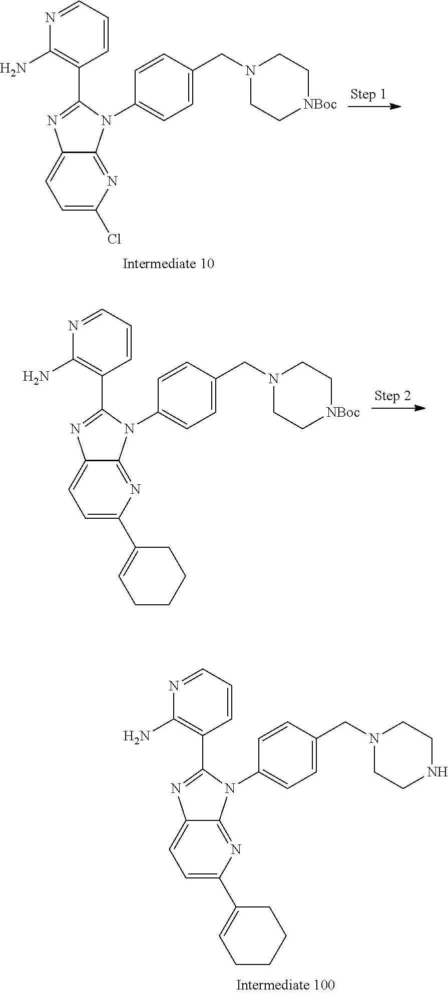 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(C5=CCCCC5)nc43)cc2)CC1 ; Nc1ncccc1-c1nc2ccc(C3=CCCCC3)nc2n1-c1ccc(CN2CCNCC2)cc1]

### Step 1: tert-Butyl 4-(4-(2-(2-aminopyridin-3-yl)-5-(cyclohex-1-en-1-yl)-3H-imidazo[4,5-b]pyridin-3-yl)benzyl)piperazine-1-carboxylate

To a solution of Intermediate 10 (200 mg, 385 μmol) in 1,4-dioxane (5 mL) and H₂O (1 mL) were added cyclohex-1-en-1-ylboronic acid (72.7 mg, 577 μmol), K₂CO₃ (159 mg, 1.15 mmol) and Pd(dppf)Cl₂ (28.1 mg, 38.5 μmol) at 25 °C under N₂. This mixture was stirred at 100 °C for 5 hr. The mixture was diluted with H₂O (5 mL) and extracted with EtOAc (20 mL×3). The combined organic layers were washed with brine (30 mL), dried over anhydrous Na₂SO₄, filtered and concentrated. The residue was purified by prep-TLC (EtOAc:petroleum ether=2:1) to give tert-butyl 4-(4-(2-(2-aminopyridin-3-yl)-5-(cyclohex-1-en-1-yl)-3H-imidazo[4,5-b]pyridin-3-yl)benzyl)piperazine-1-carboxylate (100 mg, yield: 83%) as a yellow oil. MS: m/z=566.4 [M+H]⁺.

### Step 2: 3-(5-(Cyclohex-1-en-1-yl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(cyclohex-1-en-1-yl)-3H-imidazo[4,5-b]pyridin-3-yl)benzyl)piperazine-1-carboxylate (79.7 mg, 141 μmol) in 1,4-dioxane (1 mL) was added HCl/1,4-dioxane (4 M, 35.2 IL) at 25 °C This mixture was stirred at 25 °C for 3 hr. The mixture was filtrated and concentrated. 3-(5-(Cyclohex-1-en-1-yl)-3-(4-(piperazin-1-ylmethyl)phenyl)-3H-imidazo[4,5-b]pyridin-2-yl)pyridin-2-amine (Intermediate 100, 60 mg, HCl salt, yield: 84%) was obtained as a yellow solid, which was used in the next step without purification. MS: m/z=466.2 [M+H]+. ¹H NMR (400 MHz, Methanol-d₄) δ 8.16 (d, J=8.4 Hz, 1H), 8.01 (dd, J=6.4, 1.6 Hz, 1H), 7.90 (d, J=8.4 Hz, 2H), 7.85 (dd, J=7.6, 1.4 Hz, 1H), 7.70-7.62 (m, 3H), 6.92 (dd, J=7.6, 6.4 Hz, 1H), 6.75-6.69 (m, 1H), 4.62 (s, 2H), 3.69-3.68 (m, 7H), 3.60-3.59 (m, 1H), 2.56-2.47 (m, 2H), 2.30-2.21 (m, 2H), 1.81-1.64 (m, 4H).

### Intermediate 101: 2-Hydroxy-4-(4,7-diazaspiro[2.5]octan-4-yl)benzaldehyde

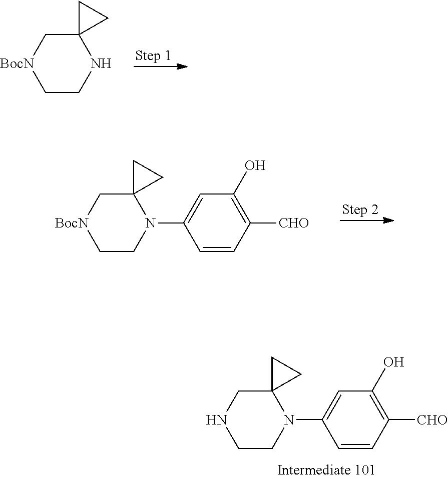 [SMILES: CC(C)(C)OC(=O)N1CCNC2(CC2)C1 ; CC(C)(C)OC(=O)N1CCN(c2ccc(C=O)c(O)c2)C2(CC2)C1 ; O=Cc1ccc(N2CCNCC23CC3)cc1O]

### Step 1: tert-Butyl 4-(4-formyl-3-hydroxyphenyl)-4,7-diazaspiro[2.5]octane-7-carboxylate

To a solution of 4-bromo-2-hydroxybenzaldehyde (80 mg, 398 μmol) in 2-methylbutan-2-ol (1 mL) were added tert-butyl 4,7-diazaspiro[2.5]octane-7-carboxylate (101 mg, 478 μmol), Pd(t-Bu₃P)₂ (20.3 mg, 39.8 μmol) and NaOtBu (115 mg, 1.19 mmol). The mixture was stirred at 100 °C for 16 hr. The mixture was concentrated, diluted with water (50 mL), and extracted with EtOAc (50 mL×2). The organic layers were dried over anhydrous Na₂SO₄, filtered and concentrated. After purified by silica gel flash chromatography (EtOAc in petroleum ether=0-33%), tert-butyl 4-(4-formyl-3-hydroxyphenyl)-4,7-diazaspiro[2.5]octane-7-carboxylate (120 mg, yield: 91%) was obtained as a yellow oil. MS: m/z=277.2 [M-C₄H₇]+.

### Step 2: 2-Hydroxy-4-(4,7-diazaspiro[2.5]octan-4-yl)benzaldehyde

To a solution of tert-butyl 4-(4-formyl-3-hydroxy-phenyl)-4,7-diazaspiro[2.5]octane-7-carboxylate (110 mg, 331 μmol) in 1,4-dioxane (0.5 mL) was added 4M HCl in 1,4-dioxane (550 μL). The mixture was stirred at 25 °C for 1 hr. The mixture was concentrated under reduced pressure to give 2-hydroxy-4-(4,7-diazaspiro[2.5]octan-4-yl)benzaldehyde (Intermediate 101, 88.9 mg, 331 μmol, yield: 100%, HCl) as a yellow oil. The crude product was used in the next step without further purification. MS: m/z=233.2 [M+H]⁺.

### Intermediate 102: 3-(3-(4-((2,6-Diazaspiro[3.4]octan-6-yl)methyl)phenyl)-5-phenyl-3H-imidazo[4,5-b]pyridin-2-yl)pyridin-2-amine

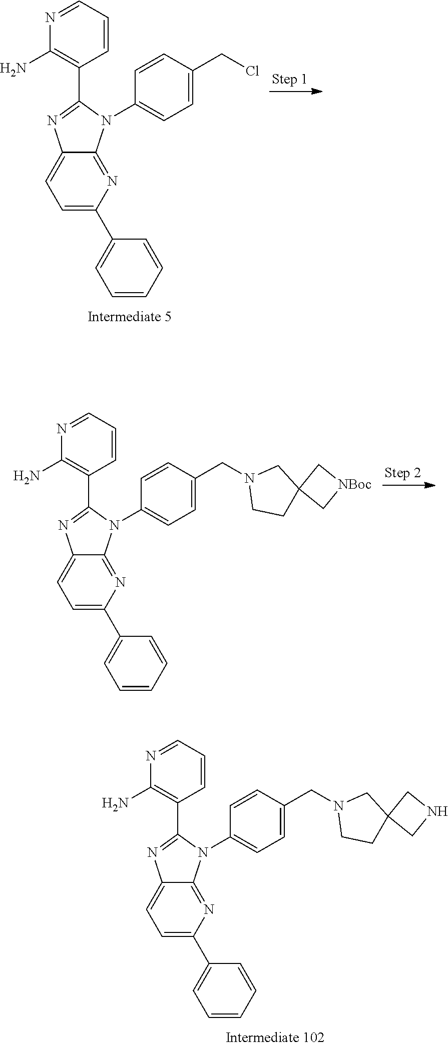 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CNC3)C2)cc1]

Following the general procedures described in Intermediate 37, the reactions were carried out using corresponding starting materials. 3-(3-(4-((2,6-Diazaspiro[3.4]octan-6-yl)methyl)phenyl)-5-phenyl-3H-imidazo[4,5-b]pyridin-2-yl)pyridin-2-amine (Intermediate 102, 17.6 mg, free base) as a yellow solid. MS: m/z=488.3 [M+H]+. ¹H NMR (400 MHz, Methanol-da) δ 8.18 (d, J=8.4 Hz, 1H), 8.06-7.89 (m, 4H), 7.51 (d, J=8.0 Hz, 2H), 7.45-7.40 (m, 3H), 7.39-7.30 (m, 3H), 6.48 (dd, J=7.6, 5.2 Hz, 1H), 3.99 (d, J=2.4 Hz, 2H), 3.72 (s, 2H), 2.87 (s, 2H), 2.71-2.61 (m, 2H), 2.18 (t, J=7.2 Hz, 2H), 1.28-1.25 (m, 2H).

### Example 1: N-(4-(2-(2-aminopyridin-3-yl)-5-phenyl-3H-imidazo[4,5-b]pyridin-3-yl)benzyl)-4-formyl-3-hydroxybenzamide

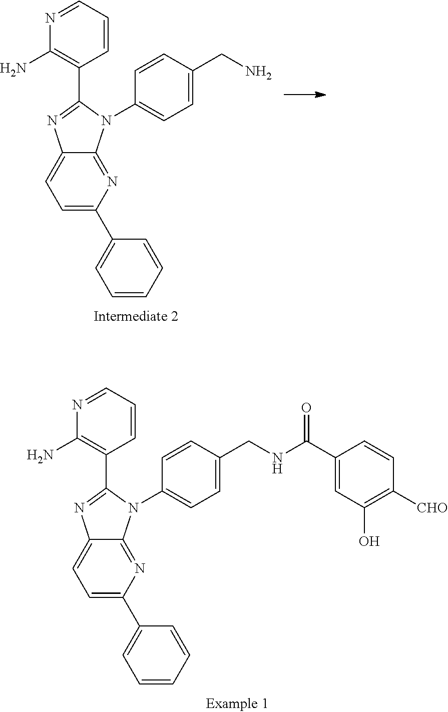 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2ccc(C=O)c(O)c2)cc1]

To a solution of Intermediate 2 (200 mg, 510 μmol) and 4-formyl-3-hydroxybenzoic acid (84.7 mg, 510 μmol) in DMF (2 mL) were added HATU (291 mg, 764 μmol) and DIPEA (329 mg, 2.55 mmol). The resulting mixture stirred at 25 °C for 16 hr. The reaction mixture was filtered. The filter liquor was concentrated to dryness. The crude product was purified by prep-HPLC (column: Waters xbridge 150×25 mm 10 μm; mobile phase: [water (NH₄HCO₃)-ACN]; B %: 30%-60%, 10 min) to give N-(4-(2-(2-aminopyridin-3-yl)-5-phenyl-3H-imidazo[4,5-b]pyridin-3-yl)benzyl)-4-formyl-3-hydroxybenzamide (Example 1, 5.4 mg, yield: 2.0%). MS: m/z=541.1 [M+H]⁺. ¹H NMR (400 MHz, Dimethylsulfoxide-d₆) δ 10.32 (s, 1H), 9.30-9.22 (m, 1H), 8.26 (d, J=8.4 Hz, 1H), 8.04-7.97 (m, 4H), 7.73 (d, J=8.0 Hz, 1H), 7.53-7.42 (m, 9H), 7.41-7.37 (m, 1H), 7.22 (d, J=6.0 Hz, 1H), 6.94 (br s, 2H), 6.42 (dd, J=7.6, 4.8 Hz, 1H), 4.60 (d, J=5.6 Hz, 2H).

### Example 2: N-(4-(2-(2-Aminopyridin-3-yl)-3H-imidazo[4,5-b]pyridin-3-yl)benzyl)-3-formyl-4-hydroxybenzamide

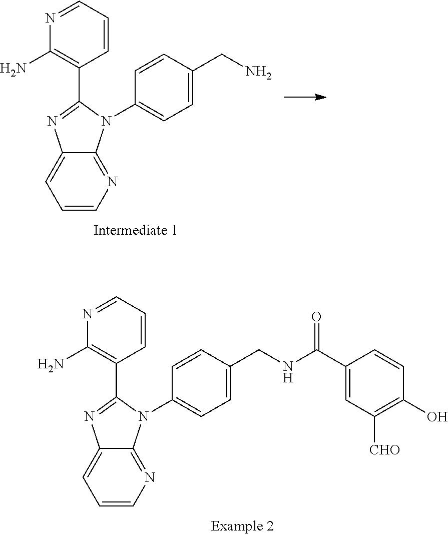 [SMILES: NCc1ccc(-n2c(-c3cccnc3N)nc3cccnc32)cc1 ; Nc1ncccc1-c1nc2cccnc2n1-c1ccc(CNC(=O)c2ccc(O)c(C=O)c2)cc1]

Following the general procedure of Example 1, the reaction of Intermediate 1 (150 mg, 478 μmol) with 3-formyl-4-hydroxy-benzoic acid (94.5 mg, 567 μmol) was carried out. After purified by prep-HPLC (neutral condition: column: Phenomenex C18 150*25 mm*10 um; mobile phase: [water (NH₄HCO₃)-ACN]; B %: 14/6-44%, 8 min) and then re-purified by another prep-HPLC (neutral condition: column: Phenomenex C18 150*25 mm*10 um; mobile phase: [water (NH₄HCO₃)-ACN]; B %: 20/6-50%, 8 min), N-(4-(2-(2-aminopyridin-3-yl)-3H-imidazo[4,5-b]pyridin-3-yl)benzyl)-3-formyl-4-hydroxybenzamide (Example 2, 6.0 mg, yield: 2.7%) was obtained. MS: m/z=465.0 [M+H]+. ¹H NMR (400 MHz, Dimethylsulfoxide-d₆) δ 10.30 (s, 1H), 9.15-8.96 (m, 1H), 8.32-8.14 (m, 3H), 8.07-7.94 (m, 2H), 7.47-7.33 (m, 5H), 7.24-7.19 (m, 2H), 7.03-6.94 (m, 3H), 6.43-6.38 (m, 1H), 4.54 (d, J=6.0 Hz, 2H).

### Example 3: N-(4-(2-(2-Aminopyridin-3-yl)-3H-imidazo[4,5-b]pyridin-3-yl)benzyl)-4-formyl-3-hydroxybenzamide

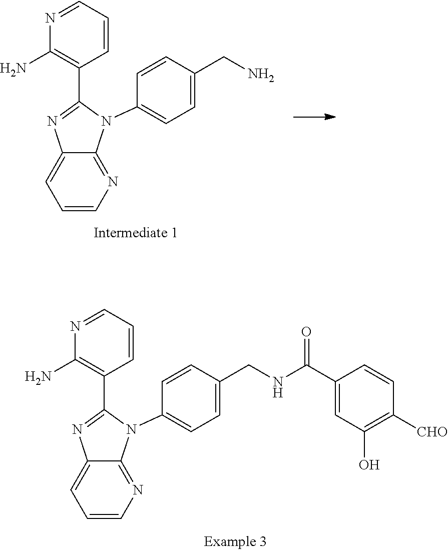 [SMILES: NCc1ccc(-n2c(-c3cccnc3N)nc3cccnc32)cc1 ; Nc1ncccc1-c1nc2cccnc2n1-c1ccc(CNC(=O)c2ccc(C=O)c(O)c2)cc1]

Following the general procedure of Example 1, the reaction of Intermediate 1 (150 mg, 478 μmol) with 4-formyl-3-hydroxybenzoic acid (94.5 mg, 567 μmol) was carried out. After purified by prep-HPLC (neutral condition: column: Waters xbridge 150*25 mm 10 um; mobile phase: [water (NH₄HCO₃)-ACN]; B %: 17%-47%, 10 min) and then re-purified by column chromatography (Eluent of 0%-15% MeOH in CH₂Cl₂), N-(4-(2-(2-aminopyridin-3-yl)-3H-imidazo[4,5-b]pyridin-3-yl)benzyl)-4-formyl-3-hydroxybenzamide (Example 3, 25 mg, yield: 11.4%) was obtained. MS: m/z=465.2 [M+H]+. ¹H NMR (400 MHz, Dimethylsulfoxide-d₆) δ 10.93 (br s, 1H), 10.42-10.27 (m, 1H), 9.34-9.14 (m, 1H), 8.35-8.27 (m, 1H), 8.20 (d, J=8.0 Hz, 1H), 8.04-7.96 (m, 1H), 7.77-7.68 (m, 1H), 7.46-7.37 (m, 6H), 7.25-7.17 (m, 2H), 6.96 (br s, 2H), 6.41 (dd, J=7.6, 4.8 Hz, 1H), 4.55 (d, J=6.0 Hz, 2H).

### Example 4: N-(4-(2-(2-Aminopyridin-3-yl)-5-phenyl-3H-imidazo[4,5-b]pyridin-3-yl)benzyl)-3-formyl-4-hydroxybenzamide

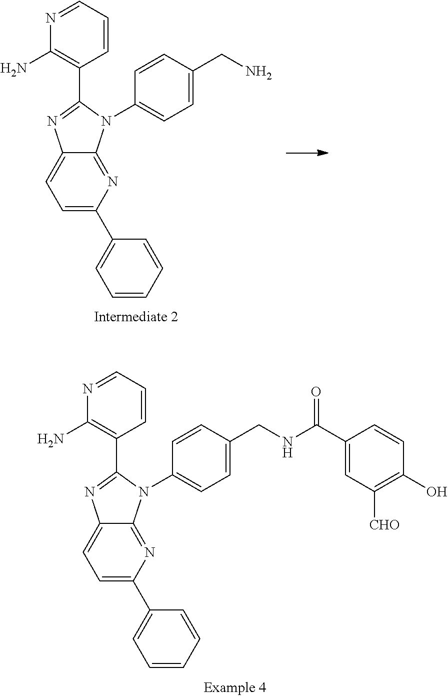 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2ccc(O)c(C=O)c2)cc1]

To a solution of Intermediate 2 (100 mg, 255 μmol) and 3-formyl-4-hydroxybenzoic acid (46.6 mg, 280 μmol) in DMF (1 mL) were added EDCI (73 mg, 382 μmol), HOBt (52 mg, 382 μmol) and DIEA (132 mg, 1.02 mmol). The mixture was stirred at 25 °C for 12 hr. The reaction mixture was filtered and concentrated under reduced pressure to give a residue. The residue was purified by prep-HPLC (column: Waters xbridge 150*25 mm 10 um; mobile phase: [water (NH₄HCO₃)-ACN]; B %: 30%-60%, 10 min) to give N-(4-(2-(2-Aminopyridin-3-yl)-5-phenyl-3H-imidazo[4,5-b]pyridin-3-yl)benzyl)-3-formyl-4-hydroxybenzamide (Example 4, 11 mg, yield: 8%). MS: m/z=541.2 [M+H]⁺. ¹H NMR (400 MHz, Dimethylsulfoxide-d₆) δ 10.32 (s, 1H), 9.16 (t, J=6.0 Hz, 1H), 8.29 (d, J=2.4 Hz, 1H), 8.26 (d, J=8.4 Hz, 1H), 8.07 (dd, J=8.8, 2.4 Hz, 1H), 8.04-7.96 (m, 5H), 7.49-7.35 (m, 7H), 7.22 (dd, J=7.6, 1.6 Hz, 1H), 7.06 (d, J=8.8 Hz, 1H), 6.94 (br s, 2H), 6.42 (dd, J=7.6, 4.8 Hz, 1H), 4.58 (d, J=6.0 Hz, 2H).

### Example 5: 5-(4-(4-(2-(2-Aminopyridin-3-yl)-5-phenyl-3H-imidazo[4,5-b]pyridin-3-yl)benzyl)piperazine-1-carbonyl)-2-hydroxybenzaldehyde

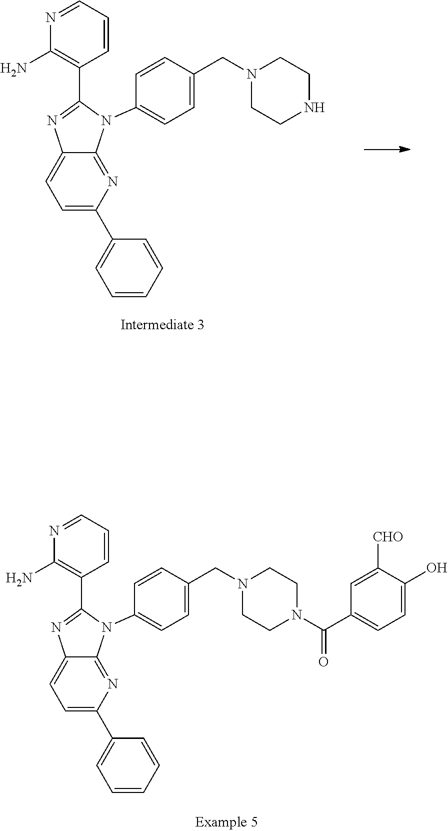 [SMILES: Nc1ncccc1-c1nc2ccc(-c3ccccc3)nc2n1-c1ccc(CN2CCNCC2)cc1 ; Nc1ncccc1-c1nc2ccc(-c3ccccc3)nc2n1-c1ccc(CN2CCN(C(=O)c3ccc(O)c(C=O)c3)CC2)cc1]

Following the general procedure of Example 4, the reaction of Intermediate 3 (150 mg, 325 μmol) with 3-formyl-4-hydroxy-benzoic acid (59 mg, 357 μmol) was carried out. After purified by prep-HPLC (column: Phenomenex luna C18 150*25 mm*10 um; mobile phase: [water (FA)-ACN]; B %: 36%-66%, 10 min), 5-(4-(4-(2-(2-aminopyridin-3-yl)-5-phenyl-3H-imidazo[4,5-b]pyridin-3-yl)benzyl)piperazine-1-carbonyl)-2-hydroxybenzaldehyde (Example 5, 32 mg, yield: 16%) was obtained. MS: m/z=610.3 [M+H]+. ¹H NMR (400 MHz, Dimethylsulfoxide-d₆) δ 10.28 (s, 1H), 8.27 (d, J=8.4 Hz, 1H), 8.06-7.95 (m, 4H), 7.68 (d, J=2.0 Hz, 1H), 7.57-7.53 (m, 1H), 7.52-7.43 (m, 7H), 7.42-7.36 (m, 1H), 7.15 (d, J=7.6 Hz, 1H), 7.04-7.00 (m, 3H), 6.38 (dd, J=7.6, 4.8 Hz, 1H), 3.63 (s, 2H), 3.58-3.49 (m, 4H), 2.47-2.42 (m, 4H).

### Example 6: 4-(4-(4-(2-(2-Aminopyridin-3-yl)-5-phenyl-3H-imidazo[4,5-b]pyridin-3-yl)benzyl)piperazine-1-carbonyl)-2-hydroxybenzaldehyde

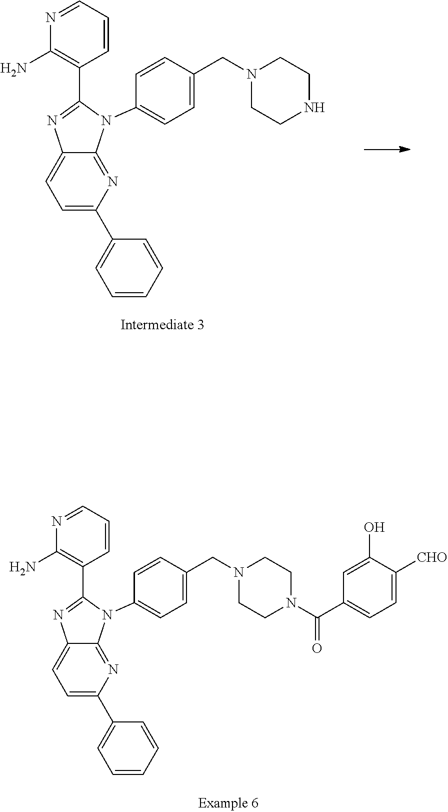 [SMILES: Nc1ncccc1-c1nc2ccc(-c3ccccc3)nc2n1-c1ccc(CN2CCNCC2)cc1 ; Nc1ncccc1-c1nc2ccc(-c3ccccc3)nc2n1-c1ccc(CN2CCN(C(=O)c3ccc(C=O)c(O)c3)CC2)cc1]

Following the general procedure of Example 4, the reaction of Intermediate 3 (50 mg, 108 μmol) with 4-formyl-3-hydroxy-benzoic acid (20 mg, 119 μmol) was carried out. After purified by prep-HPLC (column: Phenomenex luna C18 150*25 mm*10 um; mobile phase: [water (NH₄HCO₃)-ACN]; B %: 36%, 10 min), 4-(4-(4-(2-(2-aminopyridin-3-yl)-5-phenyl-3H-imidazo[4,5-b]pyridin-3-yl)benzyl)piperazine-1-carbonyl)-2-hydroxybenzaldehyde (Example 6, 15 mg, yield: 23%) was obtained. MS: m/z=610.3 [M+H]+. ¹H NMR (400 MHz, Dimethylsulfoxide-d₆) δ 10.28 (s, 1H), 8.27 (d, J=8.4 Hz, 1H), 8.05-7.95 (m, 4H), 7.68 (d, J=2.0 Hz, 1H), 7.57 (dd, J=8.4, 2.0 Hz, 1H), 7.53-7.43 (m, 7H), 7.42-7.37 (m, 1H), 7.15 (dd, J=8.0, 1.2 Hz, 1H), 7.04 (d, J=8.4 Hz, 1H), 7.02 (br s, 2H), 6.38 (dd, J=8.0, 4.8 Hz, 1H), 3.63 (s, 2H), 3.58-3.48 (m, 4H), 2.46-2.40 (m, 4H).

### Example 7: N-(1-(4-(2-(2-Aminopyridin-3-yl)-5-phenyl-3H-imidazo[4,5-b]pyridin-3-yl)benzyl)piperidin-4-yl)-3-formyl-4-hydroxybenzamide

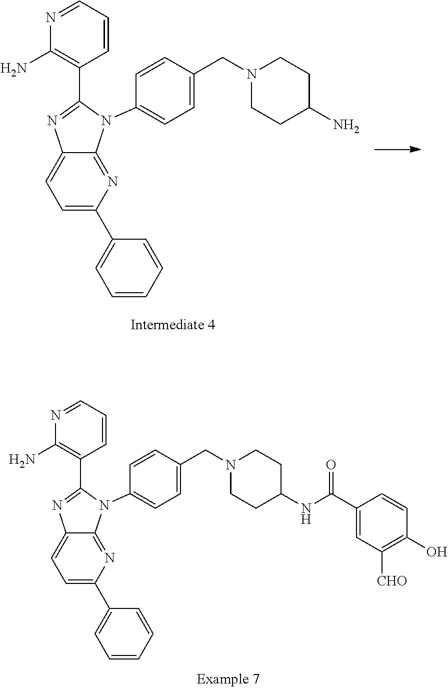 [SMILES: Nc1ncccc1-c1nc2ccc(-c3ccccc3)nc2n1-c1ccc(CN2CCC(N)CC2)cc1 ; Nc1ncccc1-c1nc2ccc(-c3ccccc3)nc2n1-c1ccc(CN2CCC(NC(=O)c3ccc(O)c(C=O)c3)CC2)cc1]

Following the general procedure of Example 4, the reaction of Intermediate 4 (150 mg, 315 μmol) with 3-formyl-4-hydroxy-benzoic acid (59 mg, 357 μmol) was carried out. After purified by prep-HPLC (column: Waters xbridge 150*25 mm 10 um; mobile phase: [water (NH₄HCO₃)-ACN]; B %: 31%-61%, 10 min), N-(1-(4-(2-(2-aminopyridin-3-yl)-5-phenyl-3H-imidazo[4,5-b]pyridin-3-yl)benzyl)piperidin-4-yl)-3-formyl-4-hydroxybenzamide (Example 7, 20 mg, yield: 10%) was obtained. MS: m/z=624.4 [M+H]⁺. ¹H NMR (400 MHz, Dimethylsulfoxide-d₆) δ 10.88 (s, 1H), 10.31 (s, 1H), 8.42 (d, J=8.0 Hz, 1H), 8.27 (d, J=8.4 Hz, 1H), 8.05-7.96 (m, 4H), 7.70 (d, J=8.0 Hz, 1H), 7.50-7.34 (m, 9H), 7.16 (d, J=7.6 Hz, 11H), 7.04 (br s, 2H), 6.38 (dd, J=7.6, 4.8 Hz, 1H), 3.85-3.72 (m, 1H), 3.58 (s, 2H), 2.93-2.83 (m, 2H), 2.14-2.04 (m, 2H), 1.84-1.76 (m, 2H), 1.67-1.58 (m, 2H).

### Example 8: N-(1-(4-(2-(2-Aminopyridin-3-yl)-5-phenyl-3H-imidazo[4,5-b]pyridin-3-yl)benzyl)piperidin-4-yl)-4-formyl-3-hydroxybenzamide

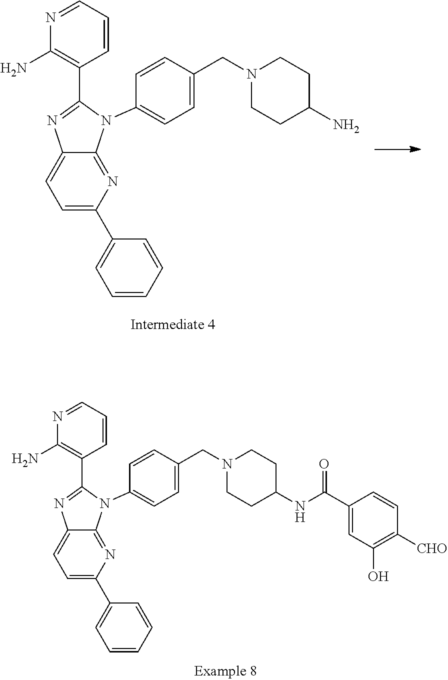 [SMILES: Nc1ncccc1-c1nc2ccc(-c3ccccc3)nc2n1-c1ccc(CN2CCC(N)CC2)cc1 ; Nc1ncccc1-c1nc2ccc(-c3ccccc3)nc2n1-c1ccc(CN2CCC(NC(=O)c3ccc(C=O)c(O)c3)CC2)cc1]

Following the general procedure of Example 4, the reaction of Intermediate 4 (150 mg, 315 μmol) with 4-formyl-3-hydroxy-benzoic acid (58 mg, 347 μmol) was carried out. After purified by prep-HPLC (column: Waters xbridge 150*25 mm 10 um; mobile phase: [water (NH₄HCO₃)-ACN]; B %: 37%-67%, 10 min), N-(1-(4-(2-(2-aminopyridin-3-yl)-5-phenyl-3H-imidazo[4,5-b]pyridin-3-yl)benzyl)piperidin-4-yl)-4-formyl-3-hydroxybenzamide (Example 8, 20 mg, yield: 10%) was obtained. MS: m/z=624.3 [M+H]⁺. ¹H NMR (400 MHz, Dimethylsulfoxide-de) δ 10.29 (s, 1H), 8.42 (d, J=8.0 Hz, 1H), 8.26 (d, J=8.4 Hz, 1H), 8.05-7.95 (m, 4H), 7.70 (d, J=8.0 Hz, 1H), 7.50-7.34 (m, 9H), 7.17-7.13 (m, 1H), 7.04 (br s, 2H), 6.36 (dd, J=7.6, 4.8 Hz, 1H), 3.83-3.74 (m, 1H), 3.60-3.55 (m, 2H), 2.91-2.83 (m, 2H), 2.14-2.06 (m, 2H), 1.84-1.75 (m, 2H), 1.67-1.55 (m, 2H).

### Example 9: 4-(4-(4-(2-(2-Aminopyridin-3-yl)-5-phenyl-3H-imidazo[4,5-b]pyridin-3-yl)benzyl)piperazin-1-yl)-2-hydroxybenzaldehyde

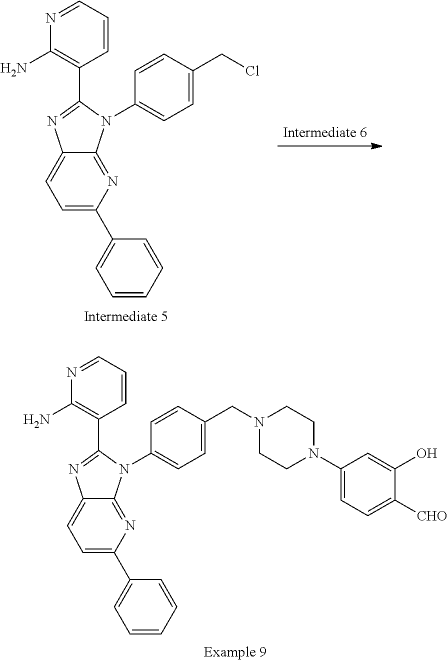 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3ccc(C=O)c(O)c3)CC2)cc1]

To a solution of Intermediate 5 (100 mg, 243 μmol) in DMF (1 mL) were added DIEA (169 μL, 971 μmol) and Intermediate 6 (59 mg, 243 μmol). The mixture was degassed and purged with N₂ three times and stirred at 80 °C for 2 hr under N₂. The reaction mixture was quenched with H₂O (20 mL) at 25 °C and extracted with CH₂Cl₂ (10 mL×3). The combined organic layers were washed with brine (10 mL×2), dried over Na₂SO₄, filtered, and concentrated under reduced pressure to give a residue. The residue was purified by prep-HPLC (column: Waters xbridge 150*25 mm 10 um; mobile phase: [water (NH₄HCO₃)-ACN]; B %: 52%-82%, 10 min) to give 4-[4-[[4-[2-(2-amino-3-pyridyl)-5-phenyl-imidazo[4,5-b]pyridin-3-yl]phenyl]methyl]piperazin-1-yl]-2-hydroxy-benzaldehyde (Example 9, 31 mg, yield: 22%). MS: m/z=582.4 [M+H]+. ¹H NMR (400 MHz, Dimethylsulfoxide-d₆) δ 11.12 (br s, 1H), 9.73 (s, 1H), 8.27 (d, J=8.4 Hz, 1H), 8.08-7.94 (m, 4H), 7.54-7.44 (m, 7H), 7.42-7.36 (m, 1H), 7.15 (dd, J=7.6, 1.6 Hz, 1H), 7.03 (br s, 2H), 6.61 (dd, J=8.8, 2.0 Hz, 1H), 6.37 (dd, J=7.6, 4.8 Hz, 1H), 6.32 (d, J=2.0 Hz, 1H), 3.64 (s, 2H), 3.45-3.39 (m, 4H), 2.54-2.51 (m, 4H).

### Example 10: 5-(4-(4-(2-(2-aminopyridin-3-yl)-5-phenyl-3H-imidazo[4,5-b]pyridin-3-yl)benzyl)piperazin-1-yl)-2-hydroxybenzaldehyde

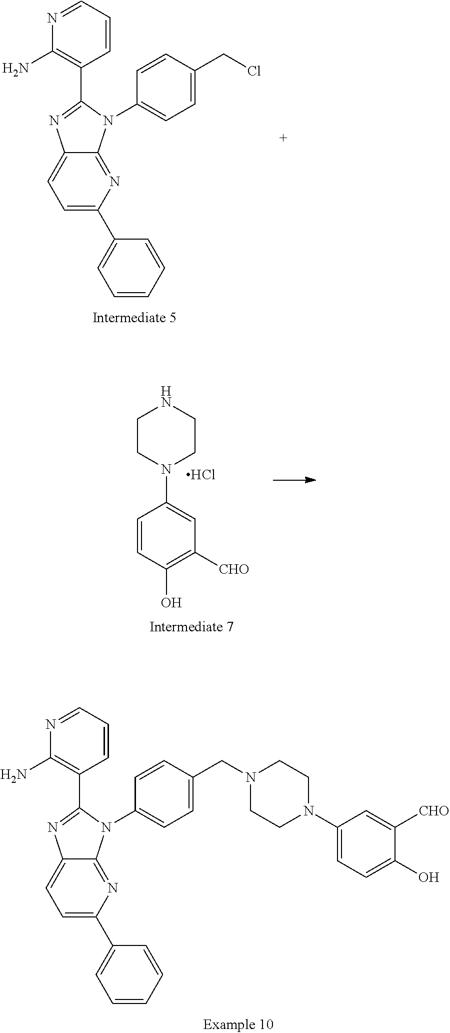 [SMILES: Nc1ncccc1-c1nc2ccc(-c3ccccc3)nc2n1-c1ccc(CCl)cc1 ; O=Cc1cc(N2CCNCC2)ccc1O ; Cl ; Nc1ncccc1-c1nc2ccc(-c3ccccc3)nc2n1-c1ccc(CN2CCN(c3ccc(O)c(C=O)c3)CC2)cc1]

To a solution of Intermediate 5 (300 mg, 728 μmol) in DMF (3 mL) were added DIEA (0.51 mL, 2.91 mmol) and Intermediate 7 (194 mg, 801 μmol). The mixture was degassed and purged with N₂ three times and stirred at 80 °C for 16 hr under N₂. The reaction mixture was quenched with H₂O (20 mL) at 25 °C and extracted with CH₂Cl₂ (10 mL×3). The combined organic layers were washed with brine (10 mL×2), dried over Na₂SO₄, filtered and concentrated under reduced pressure to give a residue. The residue was purified by prep-HPLC (column: Waters xbridge 150*25 mm 10 μm; mobile phase: [water (NH₄HCO₃)-ACN]; B %: 50% -80%, 10 min) to give 5-(4-(4-(2-(2-aminopyridin-3-yl)-5-phenyl-3H-imidazo[4,5-b]pyridin-3-yl)benzyl)piperazin-1-yl)-2-hydroxybenzaldehyde (Example 10, 44.6 mg, yield: 10%). MS: m/z=582.3 [M+H]⁺. ¹H NMR (400 MHz, Dimethylsulfoxide-d₆) 10.34-9.99 (m, 2H), 8.27 (d, J=8.4 Hz, 1H), 8.05-7.97 (m, 4H), 7.54-7.50 (m, 2H), 7.49-7.45 (m, 4H), 7.42-7.37 (m, 1H), 7.27 (dd, J=9.2, 3.2 Hz, 1H), 7.16 (dd, J=8.0, 2.0 Hz, 1H), 7.13 (d, J=3.2 Hz, 1H), 7.02 (br s, 2H), 6.91 (d, J=9.2 Hz, 1H), 6.39 (dd, J=7.6, 4.8 Hz, 1H), 3.64 (s, 2H), 3.11-3.04 (m, 4H), 2.59-2.56 (m, 4H).

### Example 11: N-(1-(4-(2-(2-aminopyridin-3-yl)-5-phenyl-3H-imidazo[4,5-b]pyridin-3-yl)phenyl)cyclobutyl)-3-formyl-4-hydroxybenzamide

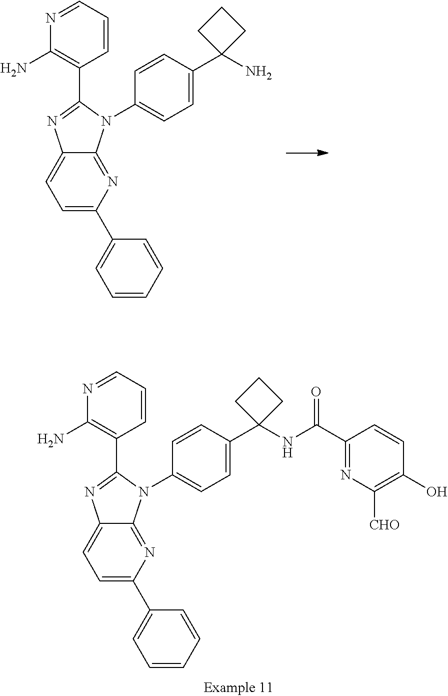 [SMILES: Nc1ncccc1-c1nc2ccc(-c3ccccc3)nc2n1-c1ccc(C2(N)CCC2)cc1 ; Nc1ncccc1-c1nc2ccc(-c3ccccc3)nc2n1-c1ccc(C2(NC(=O)c3ccc(O)c(C=O)n3)CCC2)cc1]

To a solution of 3-(3-(4-(1-aminocyclobutyl)phenyl)-5-phenyl-3H-imidazo[4,5-b]pyridin-2-yl)pyridin-2-amine (200 mg, 462 μmol) and 3-formyl-4-hydroxybenzoic acid (77 mg, 462 μmol) in dichloromethane (2 mL) were added EDCI (133 mg, 694 μmol), DIEA (239 mg, 1.85 mmol) and HOBt (94 mg, 694 μmol). The resulting mixture was stirred at 25 °C for 14 hr. The reaction mixture was diluted with H₂O (10 mL) and extracted with CH₂Cl₂ (10 mL×2). The combined organic layers were washed with brine (10 mL×2), dried over Na₂SO₄, filtered, and concentrated under reduced pressure to give a residue. The residue was purified by prep-HPLC (mobile phase: [water (HCl)-ACN]; B %: 25%-55%, 11 min) to give N-(1-(4-(2-(2-aminopyridin-3-yl)-5-phenyl-3H-imidazo[4,5-b]pyridin-3-yl)phenyl)cyclobutyl)-3-formyl-4-hydroxybenzamide (Example 11, 8.6 mg, HCl salt, yield: 3.2%). MS: m/z=581.4 [M+H]⁺. ¹H NMR (400 MHz, Methanol-di)) δ 8.29 (d, J=8.4 Hz, 1H), 8.06-7.99 (m, 3H), 7.98-7.93 (m, 2H), 7.87-7.70 (m, 5H), 7.51 (d, J=8.4 Hz, 2H), 7.45-7.37 (m, 3H), 6.87 (d, J=8.4 Hz, 1H), 6.83-6.78 (m, 1H), 2.74 (t, J=7.2 Hz, 4H), 2.27-2.15 (m, 1H), 2.10-1.99 (m, 1H).

### Example 12: N-(1-(4-(2-(2-aminopyridin-3-yl)-5-phenyl-3H-imidazo[4,5-b]pyridin-3-yl)benzyl)piperidin-4-yl)-N-cyclopropyl-3-formyl-4-hydroxybenzamide

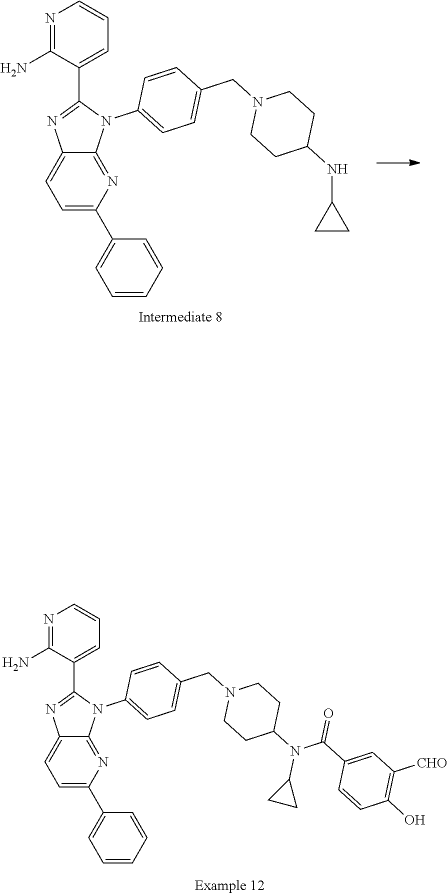 [SMILES: Nc1ncccc1-c1nc2ccc(-c3ccccc3)nc2n1-c1ccc(CN2CCC(NC3CC3)CC2)cc1 ; Nc1ncccc1-c1nc2ccc(-c3ccccc3)nc2n1-c1ccc(CN2CCC(N(C(=O)c3ccc(O)c(C=O)c3)C3CC3)CC2)cc1]

To a solution of Intermediate 8 (100 mg, 193 μmol) and 3-formyl-4-hydroxybenzoic acid (35.4 mg, 213 μmol) in DMF (2.5 mL) were added EDCI (55.7 mg, 290 μmol), HOBt (39.3 mg, 290 μmol) and DIEA (100 mg, 775 μmol) at 0 °C The mixture was stirred at 25 °C for 12 hr. The mixture was filtered and concentrated under reduced pressure. After purified by prep-HPLC (column: Waters xbridge 150×25 mm×10 μm; mobile phase: [water (NH₄HCO₃)-ACN]; gradient: 40%-70% B over 10 min), N-(1-(4-(2-(2-aminopyridin-3-yl)-5-phenyl-3H-imidazo[4,5-b]pyridin-3-yl)benzyl)piperidin-4-yl)-N-cyclopropyl-3-formyl-4-hydroxybenzamide (Example 12, 36.3 mg, yield: 28%) was obtained as a light yellow solid. MS: m/z=664.4 [M+H]+. ¹H NMR (400 MHz, Methanol-d₄) δ 10.10 (s, 1H), 8.17 (d, J=8.4 Hz, 1H), 8.05-7.90 (m, 5H), 7.71 (dd, J=8.4, 2.0 Hz, 1H), 7.55-7.52 (m, 2H), 7.46-7.35 (m, 6H), 7.30 (dd, J=7.2, 1.2 Hz, 1H), 6.98 (d, J=8.4 Hz, 1H), 6.46 (dd, J=7.6, 4.8 Hz, 1H), 4.13-4.01 (m, 1H), 3.65 (s, 2H), 3.10-3.04 (m, 2H), 2.84-2.76 (m, 1H), 2.25-2.15 (m, 4H), 1.96-1.91 (m, 2H), 0.70-0.64 (m, 2H), 0.52-0.47 (m, 2H).

### Example 13: N-(1-(4-(2-(2-Aminopyridin-3-yl)-5-phenyl-3H-imidazo[4,5-b]pyridin-3-yl)benzyl)piperidin-4-yl)-N-ethyl-3-formyl-4-hydroxybenzamide

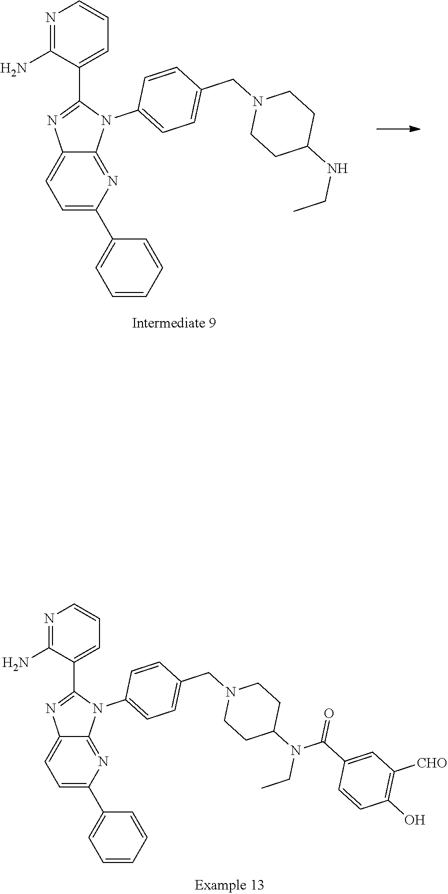 [SMILES: CCNC1CCN(Cc2ccc(-n3c(-c4cccnc4N)nc4ccc(-c5ccccc5)nc43)cc2)CC1 ; CCN(C(=O)c1ccc(O)c(C=O)c1)C1CCN(Cc2ccc(-n3c(-c4cccnc4N)nc4ccc(-c5ccccc5)nc43)cc2)CC1]

To a solution of Intermediate 9 (200 mg, 397 μmol) and 3-formyl-4-hydroxybenzoic acid (82.4 mg, 496 μmol) in DMF (4 mL) were added EDCI (114 mg, 595 μmol), HOBt (80.4 mg, 595 μmol) and DIEA (205 mg, 1.59 mmol) at 0 °C The mixture was stirred at 25 °C for 12 hr. The mixture was filtered and concentrated under reduced pressure. After purified by prep-HPLC (column: Waters xbridge 150×25 mm×10 μm; mobile phase: [water (NH₄HCO₃)-ACN]; gradient: 38%-68% B over 10 min), N-(1-(4-(2-(2-aminopyridin-3-yl)-5-phenyl-3H-imidazo[4,5-b]pyridin-3-yl)benzyl)piperidin-4-yl)-N-ethyl-3-formyl-4-hydroxybenzamide (Example 13, 20.0 mg, yield: 7.7%) was obtained as a light yellow solid. MS: m/z=652.3 [M+H]+. ¹H NMR (400 MHz, Chloroform-d) δ 11.14 (br s, 1H), 9.92 (s, 1H), 8.15-8.00 (m, 4H), 7.81 (d, J=8.4 Hz, 1H), 7.66 (d, J=2.0 Hz, 1H), 7.54 (dd, J=8.8, 2.0 Hz, 1H), 7.48-7.35 (m, 7H), 7.09 (dd, J=7.6, 1.2 Hz, 1H), 7.03 (d, J=8.4 Hz, 1H), 6.61 (br s, 2H), 6.35 (dd, J=7.6, 4.8 Hz, 1H), 3.57 (s, 2H), 3.49-3.38 (m, 2H), 3.05-2.98 (m, 2H), 2.04-1.93 (m, 4H), 1.80-1.70 (m, 3H), 1.25-1.11 (m, 3H).

### Example 14: 5-(4-(4-(2-(2-Aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde

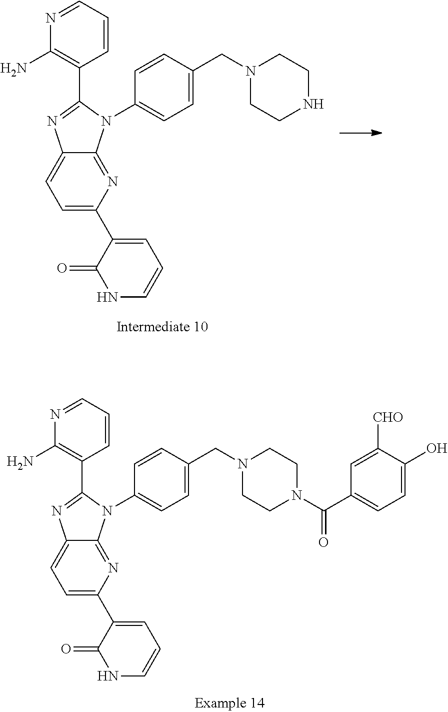 [SMILES: Nc1ncccc1-c1nc2ccc(-c3ccc[nH]c3=O)nc2n1-c1ccc(CN2CCNCC2)cc1 ; Nc1ncccc1-c1nc2ccc(-c3ccc[nH]c3=O)nc2n1-c1ccc(CN2CCN(C(=O)c3ccc(O)c(C=O)c3)CC2)cc1]

A mixture of 3-formyl-4-hydroxybenzoic acid (19.1 mg, 115 μmol), Intermediate 11 (50 mg, 104 μmol), HOBt (21.2 mg, 157 μmol), EDCI (30 mg, 157 μmol) and DIEA (67.5 mg, 522 μmol) in DMF (2 mL) was stirred at 30 °C for 16 hr. The mixture was filtered and concentrated under reduced pressure. After purified by prep-HPLC (column: Phenomenex C18 150*25 mm*10 μm; mobile phase: [water (NH₄HCO₃)-ACN]; B %: 15%-45%, over 12 min), 5-(4-(4-(2-(2-aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde (Example 14, 16.2 mg, yield: 24%) was obtained as a yellow lyophilized powder. MS: m/z=627.2 [M+H]⁺. ¹H NMR (400 MHz, Dimethylsulfoxide-d₆)S 12.23-11.75 (m, 1H), 10.28 (s, 1H), 8.68 (d, J=8.4 Hz, 1H), 8.22 (dd, J=6.8, 2.0 Hz, 1H), 8.18 (d, J=8.8 Hz, 1H), 7.98 (dd, J=4.8, 1.6 Hz, 1H), 7.68 (d, J=2.4 Hz, 1H), 7.56 (dd, J=8.4, 2.4 Hz, 1H), 7.50-7.40 (m, 5H), 7.12 (dd, J=8.0, 1.6 Hz, 1H), 7.08-6.90 (m, 3H), 6.41-6.32 (m, 2H), 3.62 (s, 2H), 3.56-3.40 (m, 4H), 2.49-2.40 (m, 4H).

### Example 15: 4-(4-(4-(2-(2-Aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde

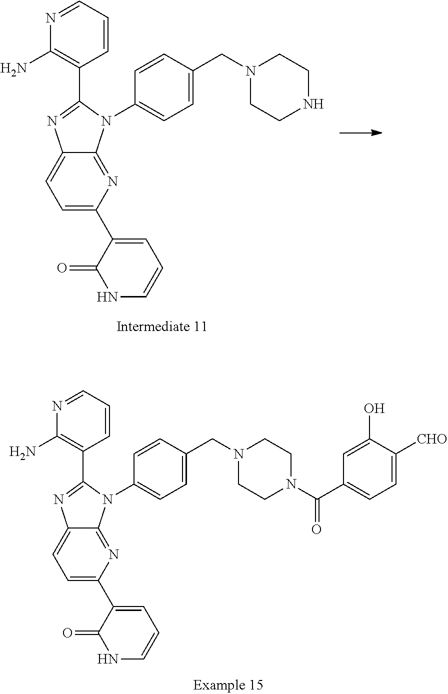 [SMILES: Nc1ncccc1-c1nc2ccc(-c3ccc[nH]c3=O)nc2n1-c1ccc(CN2CCNCC2)cc1 ; Nc1ncccc1-c1nc2ccc(-c3ccc[nH]c3=O)nc2n1-c1ccc(CN2CCN(C(=O)c3ccc(C=O)c(O)c3)CC2)cc1]

A mixture of 4-formyl-3-hydroxybenzoic acid (19.1 mg, 115 μmol), Intermediate 11 (50 mg, 104 μmol), HOBt (21.2 mg, 157 μmol), EDCI (30 mg, 157 μmol) and DIEA (67.5 mg, 522 μmol) in DMF (2 mL) was stirred at 30 °C for 16 hr. The mixture was filtered through Celite, and the filtrate was concentrated to give the crude product. After purified by prep-HPLC (column: Phenomenex C18 150*25 mm*10 μm; mobile phase: [water (NH₄HCO₃)-ACN]; B %: 17%-47%, over 12 min), 4-(4-(4-(2-(2-aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde (Example 15, 15.2 mg, yield: 22%) was obtained as a yellow lyophilized powder. MS: m/z=627.2 [M+H]⁺. ¹H NMR (400 MHz, Dimethylsulfoxide-d₆)S 12.09-11.88 (m, 1H), 11.23-10.72 (m, 1H), 10.30 (s, 1H), 8.68 (d, J=8.8 Hz, 1H), 8.23 (dd, J=7.2, 2.0 Hz, 1H), 8.19 (d, J=8.4 Hz, 1H), 7.99 (dd, J=5.2, 2.0 Hz, 1H), 7.70 (d, J=7.6 Hz, 1H), 7.51-7.47 (m, 3H), 7.46-7.43 (m, 2H), 7.13 (dd, J=8.0, 2.0 Hz, 1H), 7.03 (br s, 2H), 6.95 (s, 1H), 6.91 (d, J=8.0 Hz, 1H), 6.40-6.34 (m, 2H), 3.72-3.64 (m, 2H), 3.62 (s, 2H), 3.38-3.36 (m, 2H), 2.49-2.34 (m, 4H).

### Example 16: 4-(4-(4-(2-(2-Aminopyridin-3-yl)-3H-imidazo[4,5-b]pyridin-3-yl)benzyl)piperazin-1-yl)-2-hydroxybenzaldehyde

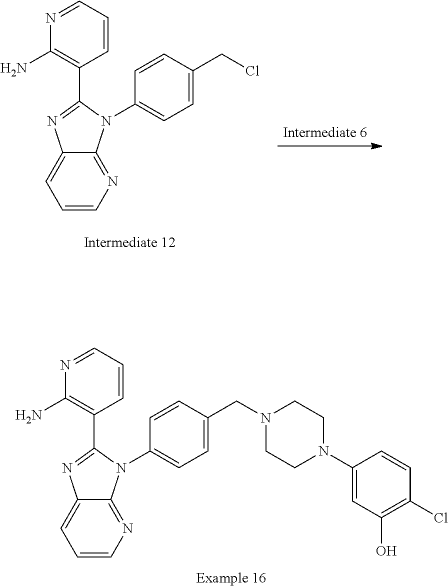 [SMILES: Nc1ncccc1-c1nc2cccnc2n1-c1ccc(CCl)cc1 ; Nc1ncccc1-c1nc2cccnc2n1-c1ccc(CN2CCN(c3ccc(Cl)c(O)c3)CC2)cc1]

To a solution of Intermediate 12 (300 mg, 893 μmol) and Intermediate 6 (221 mg, 1.1 mmol) in DMF (3 mL) were added NaI (13.4 mg, 89.3 μmol) and K₂CO₃ (370 mg, 2.7 mmol).

The mixture was stirred at 80 °C for 2 hr. The mixture was diluted with H₂O (30 mL) and extracted with EtOAc (30 mL×2). The combined organic layers were washed with brine (30 mL), dried over Na₂SO₄, filtered, and concentrated under reduced pressure. After purified by prep-HPLC (column: Waters xbridge 150×25 mm 10 μm; mobile phase: [water (NH₄HCO₃)-ACN]; gradient: 33%-63% B over 10 min), 4-(4-(4-(2-(2-aminopyridin-3-yl)-3H-imidazo[4,5-b]pyridin-3-yl)benzyl)piperazin-1-yl)-2-hydroxybenzaldehyde (Example 16, 68.1 mg, yield: 14%) was obtained as a yellow solid. MS: m/z=506.4 [M+H]⁺. ¹H NMR (400 MHz, Dimethylsulfoxide-d₆) δ 11.25-10.94 (m, 1H), 9.73 (s, 1H), 8.34 (d, J=4.8 Hz, 1H), 8.21 (d, J=8.0 Hz, 1H), 8.02-7.95 (m, 1H), 7.48 (d, J=8.0 Hz, 3H), 7.42-7.37 (m, 3H), 7.1-7.14 (m, 1H), 7.01 (br s, 2H), 6.63-6.58 (m, 1H), 6.40-6.30 (m, 2H), 3.62 (s, 2H), 3.45-3.39 (m, 4H), 2.49-2.47 (m, 4H).

### Example 17: 4-((4-(2-(2-Aminopyridin-3-yl)-5-phenyl-3H-imidazo[4,5-b]pyridin-3-yl)benzyl)(methyl)amino)-2-hydroxybenzaldehyde

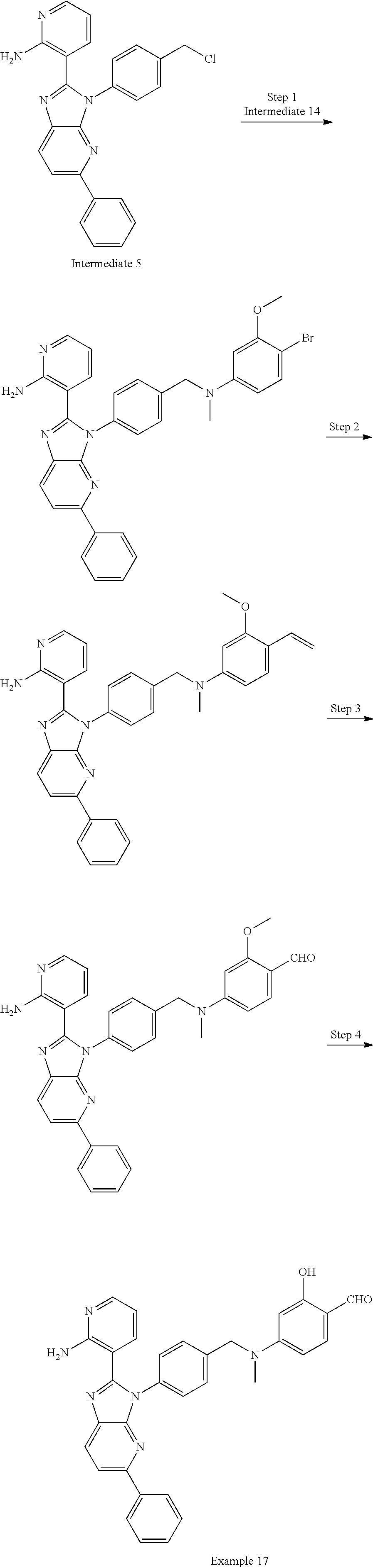 [SMILES: Nc1ncccc1-c1nc2ccc(-c3ccccc3)nc2n1-c1ccc(CCl)cc1 ; COc1cc(N(C)Cc2ccc(-n3c(-c4cccnc4N)nc4ccc(-c5ccccc5)nc43)cc2)ccc1Br ; C=Cc1ccc(N(C)Cc2ccc(-n3c(-c4cccnc4N)nc4ccc(-c5ccccc5)nc43)cc2)cc1OC ; COc1cc(N(C)Cc2ccc(-n3c(-c4cccnc4N)nc4ccc(-c5ccccc5)nc43)cc2)ccc1C=O ; CN(Cc1ccc(-n2c(-c3cccnc3N)nc3ccc(-c4ccccc4)nc32)cc1)c1ccc(C=O)c(O)c1]

### Step 1: 3-(3-(4-(((4-Bromo-3-methoxyphenyl)(methyl)amino)methyl)phenyl)-5-phenyl-3H-imidazo[4,5-b]pyridin-2-yl)pyridin-2-amine

To a solution of Intermediate 5 (133 mg, 324 μmol) and Intermediate 14 (70 mg, 324 μmol) in DMF (2 mL) were added K₂CO₃ (134 mg, 972 μmol) and NaI (4.86 mg, 32.4 μmol). The mixture was stirred at 25 °C for 16 hr. The reaction mixture was diluted with H₂O (20 mL) and extracted with EtOAc (20 mL×2). The combined organic layers were washed with brine (20 mL×2), dried over Na₂SO₄, filtered, and concentrated under reduced pressure. After purified by prep-TLC (petroleum ether:ethyl acetate=0:1), 3-(3-(4-(((4-bromo-3-methoxyphenyl)(methyl)amino)methyl)phenyl)-5-phenyl-3H-imidazo[4,5-b]pyridin-2-yl)pyridin-2-amine (19 mg, yield: 9%) was obtained as an off white solid. MS: m/z=591.1, 593.0 [M+H]⁺. ¹H NMR (400 MHz, Dimethylsulfoxide-d₆) δ 8.26 (d, J=8.4 Hz, 1H), 8.06-7.96 (m, 4H), 7.51-7.38 (m, 7H), 7.26 (d, J=8.8 Hz, 1H), 7.15 (dd, J=7.6, 1.6 Hz, 1H), 7.02 (s, 2H), 6.39-6.24 (m, 3H), 4.71 (s, 2H), 3.73 (s, 3H), 3.11 (s, 3H).

### Step 2: 3-(3-(4-(((3-Methoxy-4-vinylphenyl)(methyl)amino)methyl)phenyl)-5-phenyl-3H-imidazo[4,5-b]pyridin-2-yl)pyridin-2-amine

To a solution of 3-(3-(4-(((4-bromo-3-methoxyphenyl)(methyl)amino)methyl)phenyl)-5-phenyl-3H-imidazo[4,5-b]pyridin-2-yl)pyridin-2-amine (100 mg, 169 μmol) and 4,4,5,5-tetramethyl-2-vinyl-1,3,2-dioxaborolane (130 mg, 845 μmol) in DMF (2 mL) were added Cs₂CO₃ (165 mg, 507 μmol) and Pd(dppf)Cl₂ (12.4 mg, 16.9 μmol). The mixture was degassed, purged with N₂ three times and stirred at 100 °C for 16 hr under N₂ atmosphere. The reaction mixture was diluted with H₂O (20 mL) and extracted with EtOAc (20 mL×2). The combined organic layers were washed with brine (20 mL×2), dried over Na₂SO₄, filtered, and concentrated under reduced pressure. After purified by prep-HPLC (column: Phenomenex Cis 150×25 mm×10 μm; mobile phase: [water (NH₄HCO₃)-ACN]; gradient: 65%-95% B over 12 min), 3-(3-(4-(((3-methoxy-4-vinylphenyl)(methyl)amino)methyl)phenyl)-5-phenyl-3H-imidazo[4,5-b]pyridin-2-yl)pyridin-2-amine (5.9 mg, yield: 6.4%) was obtained as an off white solid. MS: m/z=539.3 [M+H]⁺. ¹H NMR (400 MHz, Methanol-d₄) δ 8.18 (d, J=8.4 Hz, 1H), 8.04-8.00 (m, 2H), 7.98-7.90 (m, 2H), 7.46-7.37 (m, 7H), 7.33-7.26 (m, 2H), 6.88 (dd, J=18.0, 11.2 Hz, 1H), 6.44 (dd, 1=7.6, 5.2 Hz, 1H), 6.41-6.33 (m, 1H), 6.26 (d, J=2.4 Hz, 1H), 5.55-5.46 (m, 1H), 4.96-4.92 (m, 1H), 4.69 (s, 2H), 3.72 (s, 3H), 3.15 (s, 3H).

### Step 3: 4-((4-(2-(2-Aminopyridin-3-yl)-5-phenyl-3H-imidazo[4,5-b]pyridin-3-yl)benzyl)(methyl)amino)-2-methoxybenzaldehyde

To a solution of 3-(3-(4-(((3-methoxy-4-vinylphenyl)(methyl)amino)methyl)phenyl)-5-phenyl-3H-imidazo[4,5-b]pyridin-2-yl)pyridin-2-amine (200 mg, 371 μmol) and K₂OsO₄ (4.1 mg, 11.1 μmol) in THF (9 mL) and H₂O (3 mL) was added NaIO₄ (238 mg, 1.11 mmol). The mixture was stirred at 25 °C for 1 hr. The reaction mixture was diluted with H₂O (50 mL) and extracted with EtOAc (50 mL×2). The combined organic layers were washed with brine (50 mL×2), dried over Na₂SO₄, filtered, and concentrated under reduced pressure. After purified by silica gel flash chromatography (Eluent of 50%~85% EtOAc in petroleum ether), 4-((4-(2-(2-aminopyridin-3-yl)-5-phenyl-3H-imidazo[4,5-b]pyridin-3-yl)benzyl)(methyl)amino)-2-methoxybenzaldehyde (70 mg, yield: 22%) was obtained as a yellow solid. MS: m/z=541.1 [M+H]+.

### Step 4: 4-((4-(2-(2-Aminopyridin-3-yl)-5-phenyl-3H-imidazo[4,5-b]pyridin-3-yl)benzyl)(methyl)amino)-2-hydroxybenzaldehyde

To a solution of 4-((4-(2-(2-aminopyridin-3-yl)-5-phenyl-3H-imidazo[4,5-b]pyridin-3-yl)benzyl)(methyl)amino)-2-methoxybenzaldehyde (50 mg, 92.5 μmol) in CH₂Cl₂ (10 mL) was added BBr₃ (232 mg, 925 μmol) at 0 °C The mixture was degassed, purged with N₂ three times, and stirred at 25 °C for 16 hr under N₂ atmosphere. The reaction mixture was alkalized with aqueous NaHCO₃ to pH around 9 and extracted with CH₂Cl₂ (20 mL×2). The combined organic layers were washed with brine (20 mL×2), dried over Na₂SO₄, filtered, and concentrated under reduced pressure. After purified by prep-HPLC (column: Welch Ultimate C₁₈ 150×25 mm×5 μm; mobile phase: [water (HCl)-ACN]; gradient: 51%-81% B over 10 min), 4-((4-(2-(2-aminopyridin-3-yl)-5-phenyl-3H-imidazo[4,5-b]pyridin-3-yl)benzyl)(methyl)amino)-2-hydroxybenzaldehyde (Example 17, 8.9 mg, HCl salt, yield: 18%) was obtained as a brown solid. MS: m/z=527.3 [M+H]⁺. ¹H NMR (400 MHz, Dimethylsulfoxide-d₆) δ 11.19 (br s, 1H), 9.70 (s, 1H), 8.34 (d, J=8.4 Hz, 1H), 8.27-8.13 (m, 1H), 8.12-7.84 (m, 5H), 7.77-7.68 (m, 1H), 7.58-7.37 (m, 9H), 6.82-6.74 (m, 1H), 6.46 (dd, J=8.8, 2.4 Hz, 1H), 6.15 (d, J=2.4 Hz, 1H), 4.82 (s, 2H), 3.20 (s, 3H).

### Example 18: 5-((4-(2-(2-Aminopyridin-3-yl)-5-phenyl-3H-imidazo[4,5-b]pyridin-3-yl)benzyl)(methyl)amino)-2-hydroxybenzaldehyde

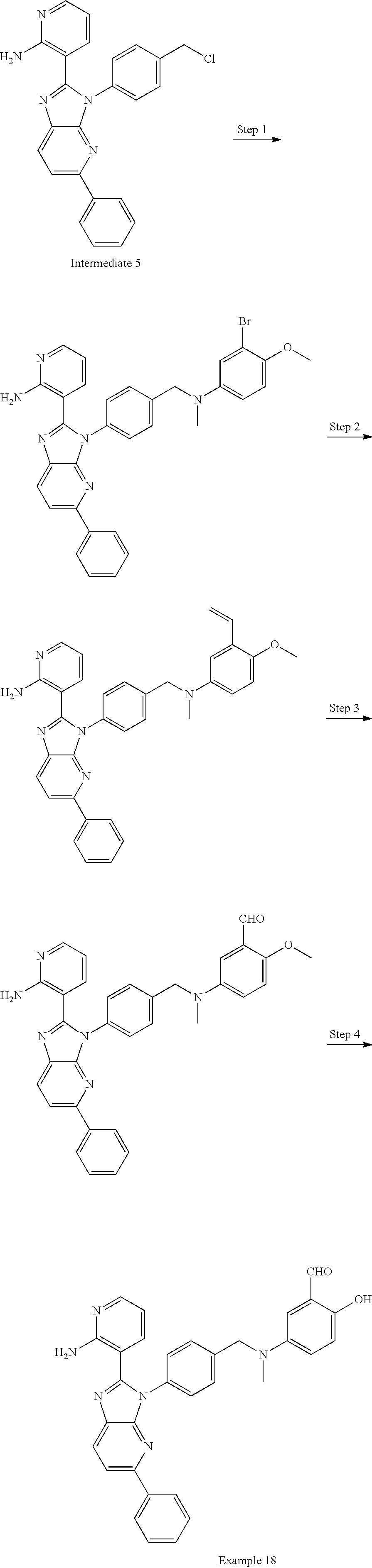 [SMILES: Nc1ncccc1-c1nc2ccc(-c3ccccc3)nc2n1-c1ccc(CCl)cc1 ; COc1ccc(N(C)Cc2ccc(-n3c(-c4cccnc4N)nc4ccc(-c5ccccc5)nc43)cc2)cc1Br ; C=Cc1cc(N(C)Cc2ccc(-n3c(-c4cccnc4N)nc4ccc(-c5ccccc5)nc43)cc2)ccc1OC ; COc1ccc(N(C)Cc2ccc(-n3c(-c4cccnc4N)nc4ccc(-c5ccccc5)nc43)cc2)cc1C=O ; CN(Cc1ccc(-n2c(-c3cccnc3N)nc3ccc(-c4ccccc4)nc32)cc1)c1ccc(O)c(C=O)c1]

Following the general procedures described in Example 17, the reactions were carried out using Intermediate 5. After purified by prep-HPLC (column: Agela DuraShell C₁₈ 150×25 mm×5 μm; mobile phase: [water (NH₃H₂O)-ACN]; gradient: 51%-81% B over 10 min), 5-((4-(2-(2-aminopyridin-3-yl)-5-phenyl-3H-imidazo[4,5-b]pyridin-3-yl)benzyl)(methyl)amino)-2-hydroxybenzaldehyde (Example 18) was obtained as a yellow solid. MS: m/z=527.2 [M+H]⁺. ¹H NMR (400 MHz, Dimethylsulfoxide-d₆) δ 10.22 (s, 1H), 9.97 (s, 1H), 8.25 (d, J=8.4 Hz, 1H), 8.05-7.97 (m, 4H), 7.49-7.38 (m, 7H), 7.16-6.87 (m, 6H), 6.43-6.26 (m, 1H), 4.53 (s, 2H), 2.97 (s, 3H).

### Example 19: N-(4-(2-(2-Aminopyridin-3-yl)-5-phenyl-3H-imidazo[4,5-b]pyridin-3-yl)benzyl)-5-formyl-2-hydroxybenzamide

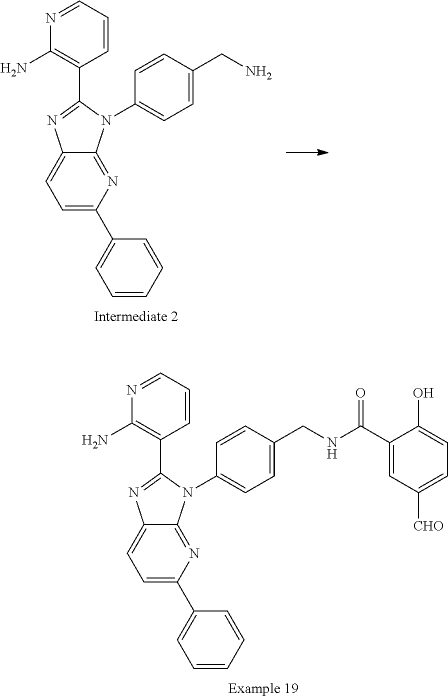 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2cc(C=O)ccc2O)cc1]

To a solution of 5-formyl-2-hydroxybenzoic acid (50.8 mg, 306 μmol) in DMF (2 mL) were added DIEA (98.8 mg, 764 μmol), HATU (145 mg, 382 μmol) and Intermediate 2 (100 mg, 255 μmol). The mixture was stirred at 20 °C for 2 hr. The reaction mixture was poured into H₂O (20 mL) at 20 °C and extracted with EtOAc (20 mL×3). The combined organic layers were washed with brine (50 mL), dried over anhydrous Na₂SO₄, filtered, and concentrated under reduced pressure. After purified by prep-HPLC (column: Phenomenex C18 75×30 mm×3 μm; mobile phase: [water(NH₃H₂O+NH₄HCO)-ACN]; B %: 17%-47%, 8 min), N-(4-(2-(2-aminopyridin-3-yl)-5-phenyl-3H-imidazo[4,5-b]pyridin-3-yl)benzyl)-5-formyl-2-hydroxybenzamide (Example 19, 9.3 mg, yield: 7%) was obtained as an off-white solid. MS: m/z=541.3 [M+H]⁺. ¹H NMR (400 MHz, Dimethylsulfoxide-d₆) δ 9.84-9.82 (m, 2H), 8.53 (s, 1H), 8.26 (d, J=8.4 Hz, 1H), 8.08-7.89 (m, 5H), 7.54-7.36 (m, 7H), 7.22 (d, J=7.6 Hz, 1H), 7.08 (d, J=8.4 Hz, 1H), 6.94 (s, 2H), 6.42 (dd, J=7.6, 4.8 Hz, 1H), 4.66 (d, J=6.0 Hz, 2H).

### Example 20: N-(4-(2-(2-Aminopyridin-3-yl)-5-phenyl-3H-imidazo[4,5-b]pyridin-3-yl)benzyl)-3-formyl-2-hydroxybenzamide

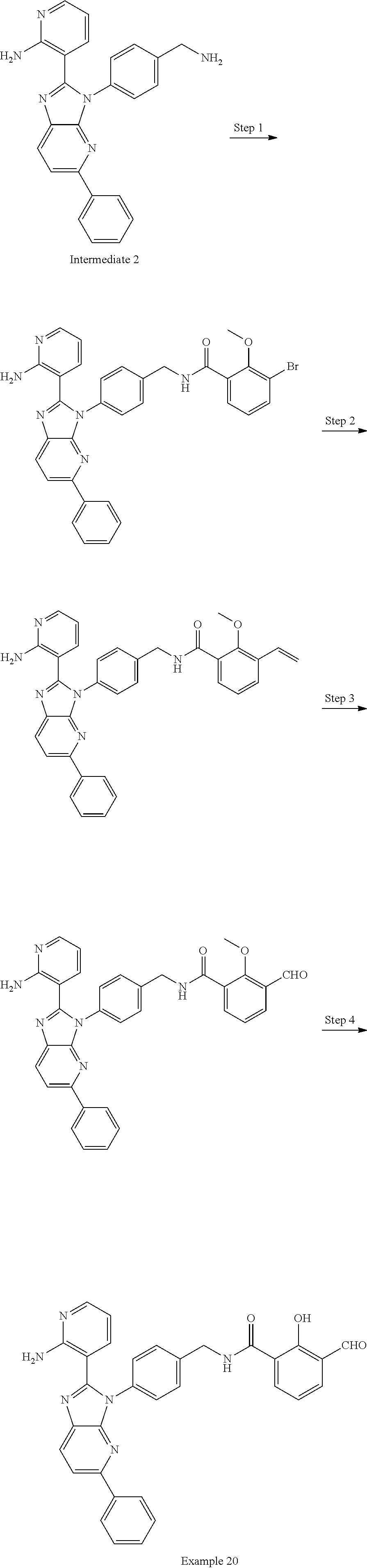 [SMILES: NCc1ccc(-n2c(-c3cccnc3N)nc3ccc(-c4ccccc4)nc32)cc1 ; COc1c(Br)cccc1C(=O)NCc1ccc(-n2c(-c3cccnc3N)nc3ccc(-c4ccccc4)nc32)cc1 ; C=Cc1cccc(C(=O)NCc2ccc(-n3c(-c4cccnc4N)nc4ccc(-c5ccccc5)nc43)cc2)c1OC ; COc1c(C=O)cccc1C(=O)NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2cccc(C=O)c2O)cc1]

### Step 1: N-(4-(2-(2-Aminopyridin-3-yl)-5-phenyl-3H-imidazo[4,5-b]pyridin-3-yl)benzyl)-3-bromo-2-methoxybenzamide

To a solution of Intermediate 2 (1.0 g, 2.6 mmol) and 3-bromo-2-methoxybenzoic acid (880 mg, 4 mmol) in DMF (10 mL) were added DIEA (1.7 g, 13 mmol) and HATU (2.9 g, 7.6 mmol). The mixture was stirred at 25 °C for 2 hr. The reaction mixture was diluted with H₂O (100 mL) and extracted with EtOAc (150 mL). The organic layer was washed with brine (90 mL), dried over Na₂SO₄, filtered, and concentrated under reduced pressure. After purified by silica gel flash chromatography (Eluent of 0-95% EtOAc in petroleum ether), N-(4-(2-(2-aminopyridin-3-yl)-5-phenyl-3H-imidazo[4,5-b]pyridin-3-yl)benzyl)-3-bromo-2-methoxybenzamide (1.4 g, yield: 74%) was obtained as a yellow solid. MS: m/z=605.1, 607.1 [M+H]⁺. ¹H NMR (400 MHz, Dimethysulfoxide-d₆) δ 9.01 (t, J=6.4 Hz, 1H), 8.27 (d, J=8.4 Hz, 1H), 8.06-7.96 (m, 4H), 7.59-7.53 (m, 2H), 7.50-7.38 (m, 6H), 7.22 (dd, J=7.6, 1.6 Hz, 1H), 7.16 (dd, J=7.6, 1.6 Hz, 1H), 7.05-6.95 (m, 3H), 6.41 (dd, J=7.6, 1.6 Hz, 1H), 4.55 (d, J=6.0 Hz, 2H), 3.88 (s, 3H).

### Step 2: N-(4-(2-(2-Aminopyridin-3-yl)-5-phenyl-3H-imidazo[4,5-b]pyridin-3-yl)benzyl)-2-methoxy-3-vinylbenzamide

A mixture of N-(4-(2-(2-aminopyridin-3-yl)-5-phenyl-3H-imidazo[4,5-b]pyridin-3-yl)benzyl)-3-bromo-2-methoxybenzamide (1.2 g, 2 mmol), 4,4,5,5-tetramethyl-2-vinyl-1,3,2-dioxaborolane (610 mg, 4 mmol), KOAc (389 mg, 4 mmol), Pd(dppf)Cl₂ (145 mg, 198 μmol) and H₂O (3 mL) in 1,4-dioxane (12 mL) was degassed and purged with N₂ three times. The mixture was stirred at 100 °C for 2 hr under N₂ atmosphere. The mixture was concentrated under reduced pressure. After purified by prep-HPLC (column: Phenomenex C18 250×50 mm×10 μm; mobile phase: [water (NH₄HCO₃)-ACN]; gradient: 40%-70% B over 8 min), N-(4-(2-(2-aminopyridin-3-yl)-5-phenyl-3H-imidazo[4,5-b]pyridin-3-yl)benzyl)-2-methoxy-3-vinylbenzamide (250 mg, yield: 21%). MS: m/z=553.4 [M+H]+. ¹H NMR (400 MHz, Methanol-d₄) δ 8.19 (d, J=8.4 Hz, 1H), 8.04 (d, J=8.4 Hz, 2H), 8.01-7.91 (m, 2H), 7.57 (d, J=8.4 Hz, 2H), 7.47 (d, J=8.4 Hz, 2H), 7.44-7.34 (m, 4H), 7.30 (t, J=8.4 Hz, 1H), 7.06 (d, J=8.4 Hz, 1H), 6.98 (d, J=8.8 Hz, 1H), 6.87-6.72 (m, 1H), 6.51-6.47 (m, 1H), 5.60 (d, J=12.6 Hz, 1H), 5.27 (d, J=12.6 Hz, 1H), 4.60 (s, 2H), 3.86 (s, 3H).

### Step 3: N-(4-(2-(2-Aminopyridin-3-yl)-5-phenyl-3H-imidazo[4,5-b]pyridin-3-yl)benzyl)-3-formyl-2-methoxybenzamide

To a solution of N-(4-(2-(2-aminopyridin-3-yl)-5-phenyl-3H-imidazo[4,5-b]pyridin-3-yl)benzyl)-2-methoxy-3-vinylbenzamide (500 mg, 905 μmol) in THF (8 mL) were added NaIO₄ (581 mg, 2.7 mmol) and K₂OsO₄·2H₂O (33 mg, 90 μmol) and H₂O (2 mL). The mixture was stirred at 25 °C for 2 hr. The reaction mixture was diluted with H₂O (20 mL) and extracted with EtOAc (60 mL). The organic layer was washed with brine (30 mL), dried over Na₂SO₄, filtered, and concentrated under reduced pressure. N-(4-(2-(2-Aminopyridin-3-yl)-5-phenyl-3H-imidazo[4,5-b]pyridin-3-yl)benzyl)-3-formyl-2-methoxybenzamide (400 mg, crude) was obtained as a black brown solid. MS: m/z=555.3 [M+H]⁺.

### Step 4: N-(4-(2-(2-Aminopyridin-3-yl)-5-phenyl-3H-imidazo[4,5-b]pyridin-3-yl)benzyl)-3-formyl-2-hydroxybenzamide

To a solution of N-(4-(2-(2-aminopyridin-3-yl)-5-phenyl-3H-imidazo[4,5-b]pyridin-3-yl)benzyl)-3-formyl-2-methoxybenzamide (400 mg, 721 μmol) in CH₂Cl₂ (4 mL) was added BBr₃ (542 mg, 2 mmol) at 0 °C The mixture was stirred at 25 °C for 3 hr. The reaction mixture was diluted with H₂O (20 mL) and extracted with EtOAc (60 mL). The organic layer was washed with brine (10 mL), dried over Na₂SO₄, filtered, and concentrated under reduced pressure to give a residue. After purified by prep-HPLC (column: Waters xbridge 150×25 mm 10 μm; mobile phase: [water (NH₄HCO₃)-ACN]; gradient: 29%-59% B over 15 min), N-(4-(2-(2-aminopyridin-3-yl)-5-phenyl-3H-imidazo[4,5-b]pyridin-3-yl)benzyl)-3-formyl-2-hydroxybenzamide (Example 20, 26.0 mg, yield: 6% for two steps) was obtained as a yellow solid. MS: m/z=541.4 [M+H]⁺. ¹H NMR (400 MHz, Dimethysulfoxide-d₆) δ 13.95 (br s, 1H), 10.36 (s, 1H), 8.38-8.17 (m, 2H), 8.08-7.95 (m, 4H), 7.90-7.75 (m, 1H), 7.65-7.29 (m, 8H), 7.22 (d, J=4.4 Hz 1H), 7.15-6.60 (m 3H), 6.42 (dd, J=7.6, 4.4 Hz, 1H), 4.67 (d, J=6.4 Hz, 2H).

### Example 21: N-(4-(2-(2-Aminopyridin-3-yl)-5-phenyl-3H-imidazo[4,5-b]pyridin-3-yl)benzyl)-2-formyl-3-hydroxybenzamide

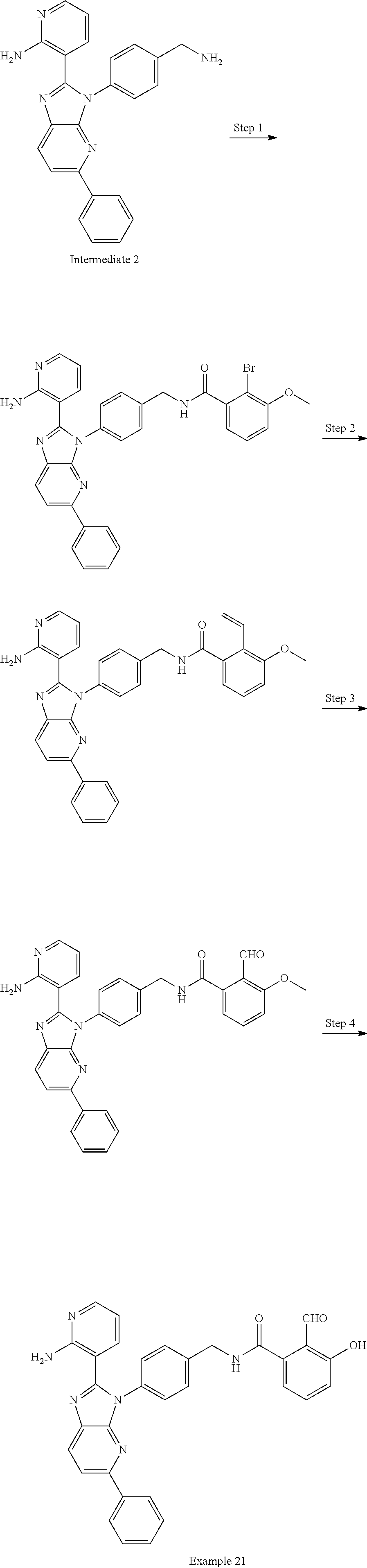 [SMILES: NCc1ccc(-n2c(-c3cccnc3N)nc3ccc(-c4ccccc4)nc32)cc1 ; COc1cccc(C(=O)NCc2ccc(-n3c(-c4cccnc4N)nc4ccc(-c5ccccc5)nc43)cc2)c1Br ; C=Cc1c(OC)cccc1C(=O)NCc1ccc(-n2c(-c3cccnc3N)nc3ccc(-c4ccccc4)nc32)cc1 ; COc1cccc(C(=O)NCc2ccc(-n3c(-c4cccnc4N)nc4ccc(-c5ccccc5)nc43)cc2)c1C=O ; Nc1ncccc1-c1nc2ccc(-c3ccccc3)nc2n1-c1ccc(CNC(=O)c2cccc(O)c2C=O)cc1]

Following the general procedures described in Example 20, the reactions were carried out using Intermediate 2. After purified by prep-TLC (SiO₂, CH₂Cl₂:MeOH=15:1), N-(4-(2-(2-aminopyridin-3-yl)-5-phenyl-3H-imidazo[4,5-b]pyridin-3-yl)benzyl)-2-formyl-3-hydroxybenzamide (Example 21) was obtained as an off white solid. MS: m/z=541.4 [M+H]⁺. ¹H NMR (400 MHz, Dimethysulfoxide-d₆) δ 10.17-9.75 (m, 1H), 8.26 (d, J=8.4 Hz, 1H), 8.06-7.95 (m, 4H), 7.49-7.42 (m, 6H), 7.41-7.34 (m, 2H), 7.20-7.10 (m, 2H), 7.06-6.93 (m, 3H), 6.60-6.50 (m, 1H), 6.41 (dd, J=7.6, 4.8 Hz, 1H), 5.74 (d, J=8.4 Hz, 1H), 4.97 (d, J=16.0 Hz, 1H), 4.45 (d, J=16.0 Hz, 1H).

### Example 22: 2-Amino-N-(4-(2-(2-aminopyridin-3-yl)-5-phenyl-3H-imidazo[4,5-b]pyridin-3-yl)benzyl)-5-formyl-4-hydroxybenzamide

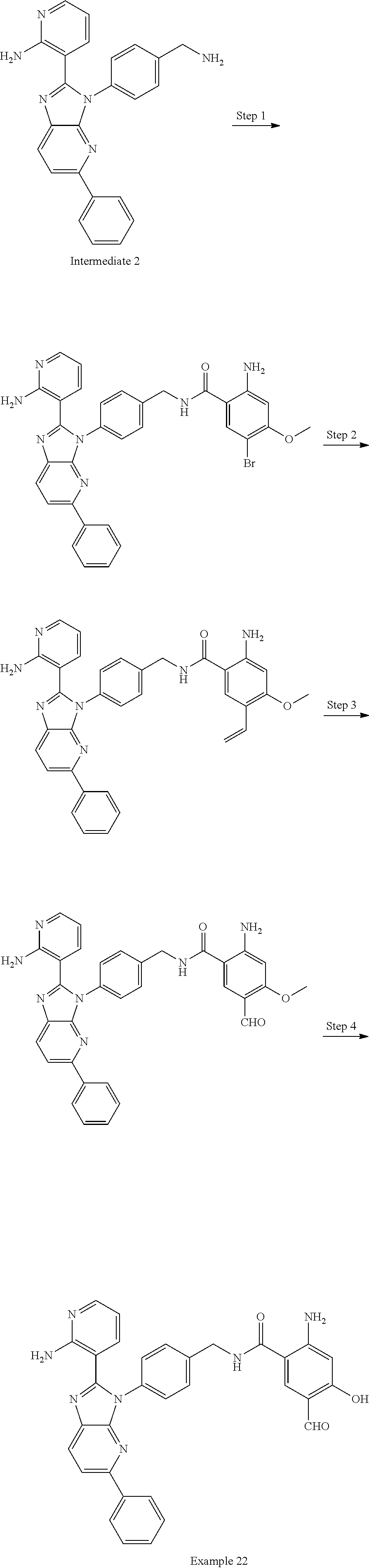 [SMILES: NCc1ccc(-n2c(-c3cccnc3N)nc3ccc(-c4ccccc4)nc32)cc1 ; COc1cc(N)c(C(=O)NCc2ccc(-n3c(-c4cccnc4N)nc4ccc(-c5ccccc5)nc43)cc2)cc1Br ; C=Cc1cc(C(=O)NCc2ccc(-n3c(-c4cccnc4N)nc4ccc(-c5ccccc5)nc43)cc2)c(N)cc1OC ; COc1cc(N)c(C(=O)NCc2ccc(-n3c(-c4cccnc4N)nc4ccc(-c5ccccc5)nc43)cc2)cc1C=O ; Nc1cc(O)c(C=O)cc1C(=O)NCc1ccc(-n2c(-c3cccnc3N)nc3ccc(-c4ccccc4)nc32)cc1]

### Step 1: 2-Amino-N-(4-(2-(2-aminopyridin-3-yl)-5-phenyl-3H-imidazo[4,5-b]pyridin-3-yl)benzyl)-5-bromo-4-methoxybenzamide

To a solution of Intermediate 2 (200 mg, 510 μmol) and 2-amino-5-bromo-4-methoxybenzoic acid (138 mg, 561 μmol) in DMF (2 mL) were added HATU (291 mg, 764 μmol) and DIEA (329 mg, 2.55 mmol). The mixture was stirred at 25 °C for 16 hr. The reaction mixture was diluted with H₂O (10 mL) and extracted with CH₂Cl₂ (10 mL×2). The combined organic layers were washed with brine (10 mL), dried over Na₂SO₄, filtered, and concentrated under reduced pressure. After purified by prep-HPLC (column: Waters xbridge 150×25 mm×10 μm; mobile phase: [water (NH₄HCO₃)-ACN]; gradient: 46%-76% B, 10 min), 2-amino-N-(4-(2-(2-aminopyridin-3-yl)-5-phenyl-3H-imidazo[4,5-b]pyridin-3-yl)benzyl)-5-bromo-4-methoxybenzamide (25.3 mg, yield: 8%) was obtained as a yellow solid. MS: m/z=620.1, 622.1 [M+H]⁺. ¹H NMR (400 MHz, Dimethylsulfoxide-d₆) δ 8.83 (t, J=5.6 Hz, 1H), 8.26 (d, J=8.0 Hz, 1H), 8.06-7.96 (m, 4H), 7.86 (s, 1H), 7.51-7.42 (m, 6H), 7.41-7.37 (m, 1H), 7.21 (d, J=7.6 Hz, 1H), 6.95 (s, 2H), 6.85 (s, 2H), 6.47-6.39 (m, 2H), 4.51 (d, J=5.6 Hz, 2H), 3.78 (s, 3H).

### Step 2: 2-Amino-N-(4-(2-(2-aminopyridin-3-yl)-5-phenyl-3H-imidazo[4,5-b]pyridin-3-yl)benzyl)-4-methoxy-5-vinylbenzamide

To a solution of 2-amino-N-(4-(2-(2-aminopyridin-3-yl)-5-phenyl-3H-imidazo[4,5-b]pyridin-3-yl)benzyl)-5-bromo-4-methoxybenzamide (360 mg, 581 mol) and Pd(dba)₂ (16.7 mg, 29 μmol) in toluene (5 mL) was added tributyl(vinyl)stannane (780 mg, 2.46 mmol). The mixture was degassed and purged with N₂ three times and stirred at 110 °C for 16 hr under N₂ atmosphere. The reaction mixture was quenched with aqueous KF (10 mL) at 25 °C, diluted with H₂O (50 mL), and extracted with EtOAc (50 mL×2). The combined organic layers were washed with brine (50 mL), dried over Na₂SO₄, filtered, and concentrated under reduced pressure. After purified by silica gel flash chromatography (Eluent of 50%-100% EtOAc in petroleum ether), 2-amino-N-(4-(2-(2-aminopyridin-3-yl)-5-phenyl-3H-imidazo[4,5-b]pyridin-3-yl)benzyl)-4-methoxy-5-vinylbenzamide (200 mg, yield: 42%) was obtained as a yellow solid. MS: m/z=568.2 [M+H]⁺.

### Step 3: 2-Amino-N-(4-(2-(2-aminopyridin-3-yl)-5-phenyl-3H-imidazo[4,5-b]pyridin-3-yl)benzyl)-5-formyl-4-methoxybenzamide

To a solution of 2-amino-N-(4-(2-(2-aminopyridin-3-yl)-5-phenyl-3H-imidazo[4,5-b]pyridin-3-yl)benzyl)-4-methoxy-5-vinylbenzamide (200 mg, 352 μmol) and K₂OsO₄ (3.89 mg, 10.6 μmol) in THF (9 mL) and H₂O (3 mL) was added NaIO₄ (226 mg, 1.06 mmol). The mixture was stirred at 25 °C for 1 hr. The reaction mixture was diluted with H₂O (50 mL) and extracted with EtOAc (50 mL×2). The combined organic layers were washed with brine (50 mL×2), dried over Na₂SO₄, filtered, and concentrated under reduced pressure. After purified by silica gel flash chromatography (Eluent of 50%-100% EtOAc in petroleum ether), 2-amino-N-(4-(2-(2-aminopyridin-3-yl)-5-phenyl-3H-imidazo[4,5-b]pyridin-3-yl)benzyl)-5-formyl-4-methoxybenzamide (50 mg, yield: 18%) was obtained as a yellow solid. MS: m/z=570.1 [M+H]+.

### Step 4: 2-Amino-N-(4-(2-(2-aminopyridin-3-yl)-5-phenyl-3H-imidazo[4,5-b]pyridin-3-yl)benzyl)-5-formyl-4-hydroxybenzamide

To a solution of 2-amino-N-(4-(2-(2-aminopyridin-3-yl)-5-phenyl-3H-imidazo[4,5-b]pyridin-3-yl)benzyl)-5-formyl-4-methoxybenzamide (40 mg, 70.2 μmol) in CH₂Cl₂ (10 mL) was added BBr₃ (176 mg, 702 μmol) at 0 °C The mixture degassed, purged with N₂ three times, and stirred at 25 °C for 16 hr under N₂ atmosphere. The reaction mixture was alkalized with aqueous NaHCO₃ to pH around 9 and extracted with CH₂Cl₂ (20 mL×2). The combined organic layers were washed with brine (20 mL×2), dried over Na₂SO₄, filtered, and concentrated under reduced pressure. After purified by prep-TLC (Petroleum ether:EtOAc=0:1), 2-amino-N-(4-(2-(2-aminopyridin-3-yl)-5-phenyl-3H-imidazo[4,5-b]pyridin-3-yl)benzyl)-5-formyl-4-hydroxybenzamide (Example 22, 3.5 mg, yield: 8%) was obtained as a yellow solid. MS: m/z=556.3 [M+H]+. ¹H NMR (400 MHz, Methanol-d₄) δ 9.57 (s, 1H), 8.25 (d, J=8.8 Hz, 1H), 8.05-7.94 (m, 5H), 7.67 (d, J=7.2 Hz, 1H), 7.62-7.58 (m, 2H), 7.48-7.31 (m, 6H), 6.72-6.61 (m, 1H), 4.67 (s, 2H).

### Example 23: 3-(4-(4-(2-(2-aminopyridin-3-yl)-5-phenyl-3H-imidazo[4,5-b]pyridin-3-yl)benzyl)piperazin-1-yl)-2-hydroxybenzaldehyde

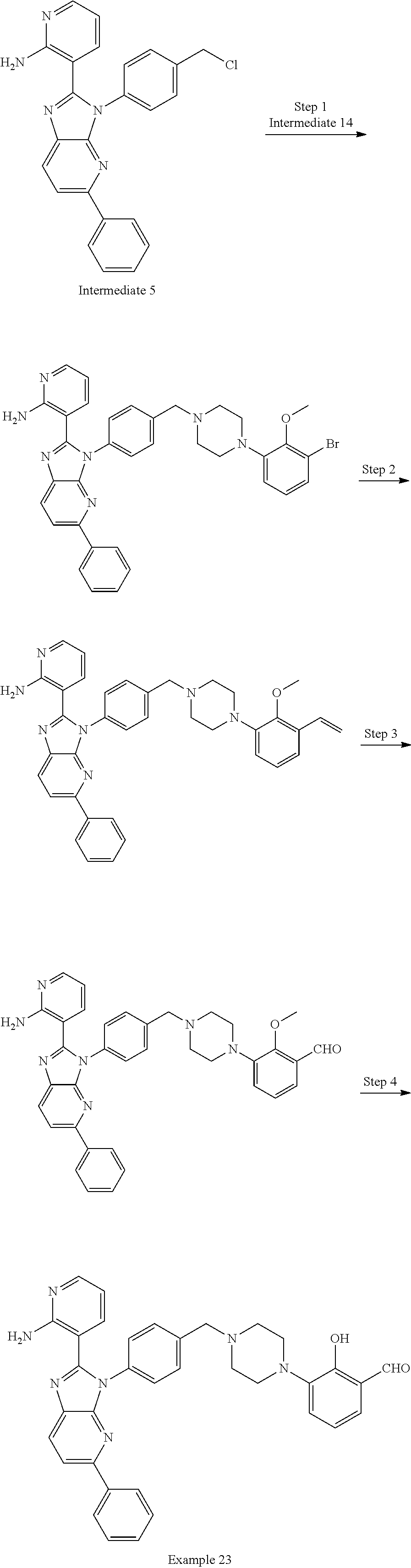 [SMILES: Nc1ncccc1-c1nc2ccc(-c3ccccc3)nc2n1-c1ccc(CCl)cc1 ; COc1c(Br)cccc1N1CCN(Cc2ccc(-n3c(-c4cccnc4N)nc4ccc(-c5ccccc5)nc43)cc2)CC1 ; C=Cc1cccc(N2CCN(Cc3ccc(-n4c(-c5cccnc5N)nc5ccc(-c6ccccc6)nc54)cc3)CC2)c1OC ; COc1c(C=O)cccc1N1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCN(c3cccc(C=O)c3O)CC2)cc1]

### Step 1: 3-(3-(4-((4-(3-Bromo-2-methoxyphenyl)piperazin-1-yl)methyl)phenyl)-5-phenyl-3H-imidazo[4,5-b]pyridin-2-yl)pyridin-2-amine

To a solution of Intermediate 14 (395 mg, 1.46 mmol) and 3-(3-(4-ethylphenyl)-5-phenyl-3H-imidazo[4,5-b]pyridin-2-yl)pyridin-2-amine (500 mg, 1.21 mmol) in DMF (10 mL) were added NaI (91 mg, 607 μmol) and K₂CO₃ (671 mg, 4.86 mmol). The mixture was stirred at 25 °C for 16 hr. The reaction mixture was diluted with H₂O (50 mL) and extracted with EtOAc (30 mL×3). The combined organic layers were washed with brine (50 mL), dried over Na₂SO₄, filtered and concentrated under reduced pressure. After purified by silica gel flash chromatography (Eluent of 0-7% MeOH in CH₂Cl₂), 3-(3-(4-((4-(3-bromo-2-methoxyphenyl)piperazin-1-yl)methyl)phenyl)-5-phenyl-3H-imidazo[4,5-b]pyridin-2-yl)pyridin-2-amine (7.3 mg, yield: 38%) was obtained as a yellow solid. MS: m/z=646.2, 648.2 [M+H]⁺. ¹H NMR (400 MHz, Methanol-d₄) δ 8.20 (d, J=8.4 Hz, 1H), 8.06-8.03 (m, 2H), 8.00-7.97 (m, 2H), 7.95 (d, J=8.8 Hz, 1H), 7.59 (d, J=8.8 Hz, 2H), 7.48-7.35 (m, 5H), 7.17 (dd, J=7.2, 2.8 Hz, 1H), 6.96-6.89 (m, 2H), 6.49 (dd, J=7.6, 5.2 Hz, 1H), 3.84 (s, 3H), 3.71 (s, 2H), 3.19-3.16 (m, 4H), 2.76-2.70 (m, 4H).

### Step 2: 3-(3-(4-((4-(2-Methoxy-3-vinylphenyl)piperazin-1-yl)methyl)phenyl)-5-phenyl-3H-imidazo[4,5-b]pyridin-2-yl)pyridin-2-amine

A mixture of 3-(3-(4-((4-(3-bromo-2-methoxyphenyl)piperazin-1-yl)methyl)phenyl)-5-phenyl-3H-imidazo[4,5-b]pyridin-2-yl)pyridin-2-amine (230 mg, 356 μmol), 4,4,5,5-tetramethyl-2-vinyl-1,3,2-dioxaborolane (65.7 mg, 427 μmol), Pd(dppf)Cl₂ (26 mg, 35.6 μmol) and Cs₂CO₃ (34.8 mg, 1.07 mmol) in 1,4-dioxane (5 mL) and H₂O (0.5 mL) was degassed and purged with N₂ three times. The mixture was stirred at 100 °C for 16 hr under N₂ atmosphere. The reaction mixture was diluted with water (100 mL) and extracted with EtOAc (50 mL×3). The combined organic layers were washed with brine (100 mL), dried over Na₂SO₄, filtered and concentrated under reduced pressure. After purified by silica gel flash chromatography (Eluent of 0~9% MeOH in CH₂Cl₂), 3-(3-(4-((4-(2-methoxy-3-vinylphenyl)piperazin-1-yl)methyl)phenyl)-5-phenyl-3H-imidazo[4,5-b]pyridin-2-yl)pyridin-2-amine (177 mg, yield: 71%) was obtained as a yellow solid. MS: m/z=594.3 [M+H]⁺.

### Step 3: 3-(4-(4-(2-(2-Aminopyridin-3-yl)-5-phenyl-3H-imidazo[4,5-b]pyridin-3-yl)benzyl)piperazin-1-yl)-2-methoxybenzaldehyde

To a mixture of 3-(3-(4-((4-(2-methoxy-3-vinylphenyl)piperazin-1-yl)methyl)phenyl)-5-phenyl-3H-imidazo[4,5-b]pyridin-2-yl)pyridin-2-amine (150 mg, 253 μmol) in THF (3 mL) and H₂O (1 mL) were added NaIO₄ (162 mg, 756 μmol) and K₂OsO₄ (9.31 mg, 25.3 μmol). The mixture was stirred at 0 °C for 2 hr. The reaction mixture was quenched with Na₂SO₃ (5 mL) and NaHCO₃ (5 mL) at 0 °C The reaction mixture was diluted with water (100 mL) and extracted with EtOAc (50 mL×3). The combined organic layers were washed with brine (100 mL), dried over Na₂SO₄, filtered and concentrated under reduced pressure. After purified by prep-HPLC (column: Waters xbridge 150×25 mm 10 μm; mobile phase: [water (NH₄HCO₃)-ACN]; gradient: 60%-90% B over 53 min), 3-(4-(4-(2-(2-aminopyridin-3-yl)-5-phenyl-3H-imidazo[4,5-b]pyridin-3-yl)benzyl)piperazin-1-yl)-2-methoxybenzaldehyde (6.4 mg, yield: 4%) was obtained as a yellow solid. MS: m/z=596.4 [M+H]⁺, ¹H NMR (400 MHz, Methanol-d₄) δ 10.38 (s, 1H), 8.20 (d, J=8.4 Hz, 1H), 8.04 (d, J=7.6 Hz, 2H), 7.99 (d, J=4.0 Hz, 1H), 7.95 (d, J=8.4 Hz, 1H), 7.60 (d, J=8.0 Hz, 2H), 7.49-7.34 (m, 8H), 7.18 (d, J=8.4 Hz, 1H), 6.48 (dd, J=7.2, 4.4 Hz, 1H), 4.01 (s, 3H), 3.73 (s, 2H), 3.23-3.16 (m, 4H), 2.78-2.73 (m, 4H).

### Step 4: 3-(4-(4-(2-(2-Aminopyridin-3-yl)-5-phenyl-3H-imidazo[4,5-b]pyridin-3-yl)benzyl)piperazin-1-yl)-2-hydroxybenzaldehyde

To a solution of 3-(4-(4-(2-(2-aminopyridin-3-yl)-5-phenyl-3H-imidazo[4,5-b]pyridin-3-yl)benzyl)piperazin-1-yl)-2-methoxybenzaldehyde (86 mg, 144 μmol) in CH₂Cl₂ (5 mL) was added BBr₃ (43.4 mg, 173 μmol). The mixture was stirred at 0 °C for 2 hr. The reaction mixture was quenched with H₂O (30 mL) at 25 °C, diluted with NaHCO₃ (20 mL), and extracted with EtOAc (3×30 mL). The combined organic layers was dried over Na₂SO₄, filtered and concentrated under reduced pressure. 3-(4-(4-(2-(2-Aminopyridin-3-yl)-5-phenyl-3H-imidazo[4,5-b]pyridin-3-yl)benzyl)piperazin-1-yl)-2-hydroxybenzaldehyde (Example 23, 5.3 mg, yield: 6%) was obtained as a yellow solid. MS: m/z=582.4 [M+H]⁺. ¹H NMR (400 MHz, Methanol-d₄) δ 9.97 (s, 1H), 8.20 (d, J=8.4 Hz, 1H), 8.04 (d, J=7.6 Hz, 2H), 7.99-7.93 (m, 2H), 7.59 (d, J=8.4 Hz, 2H), 7.47-7.28 (m, 8H), 7.05-6.97 (m, 1H), 6.52-6.47 (m, 1H), 3.73 (s, 2H), 3.18-3.13 (m, 4H), 2.78-2.72 (m, 4H).

### Example 24: 4-(4-(4-(2-(2-Aminopyridin-3-yl)-5-phenyl-3H-imidazo[4,5-b]pyridin-3-yl)benzyl)piperazin-1-yl)-2-fluoro-6-hydroxybenzaldehyde

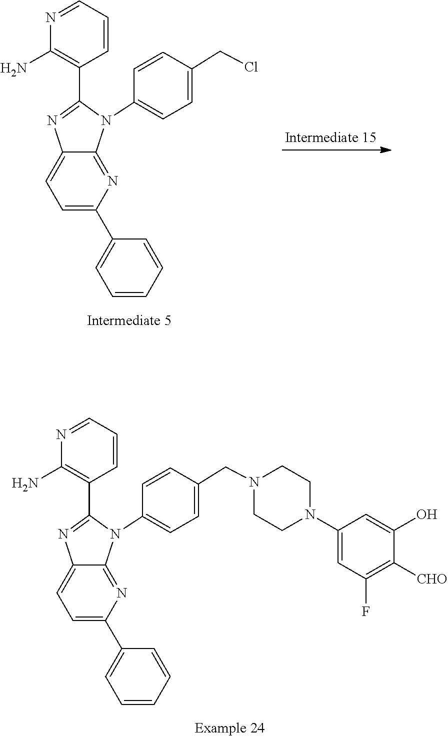 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3cc(O)c(C=O)c(F)c3)CC2)cc1]

To a solution of Intermediate 5 (150 mg, 334 μmol) and Intermediate 15 (113 mg, 435 μmol, HCl salt) in DMF (1.5 mL) were added NaI (25.1 mg, 167 μmol) and K₂CO₃ (231 mg, 1.67 mmol). The mixture was stirred at 80 °C for 2 hr. The reaction mixture was filtered. After purified by prep-HPLC (column: [water (NH₄HCO₃)-ACN]; B %: 52%-82%, 18 min), 4-(4-(4-(2-(2-aminopyridin-3-yl)-5-phenyl-3H-imidazo[4,5-b]pyridin-3-yl)benzyl)piperazin-1-yl)-2-fluoro-6-hydroxybenzaldehyde (Example 24, 27.4 mg, yield: 13.4%) was obtained as a light yellow solid. MS: m/z=600.2 [M+H]+. ¹H NMR (400 MHz, Methanol-d₄) δ 9.84 (s, 1H), 8.20 (d, J=8.4 Hz, 1H), 8.07-7.92 (m, 4H), 7.60-7.53 (m, 2H), 7.49-7.31 (m, 6H), 6.50-6.45 (m, 1H), 6.31 (dd, J=15.2, 2.4 Hz, 1H), 6.13 (d, J=2.0 Hz, 1H), 3.69 (s, 2H), 3.52-3.47 (m, 4H), 2.65-2.59 (m, 4H). ¹⁹F NMR (400 MHz, Methanol-d₄) δ −123.080.

### Example 25: 4-(4-(4-(2-(2-Aminopyridin-3-yl)-5-phenyl-3H-imidazo[4,5-b]pyridin-3-yl)benzyl)piperazin-1-yl)-5-fluoro-2-hydroxybenzaldehyde

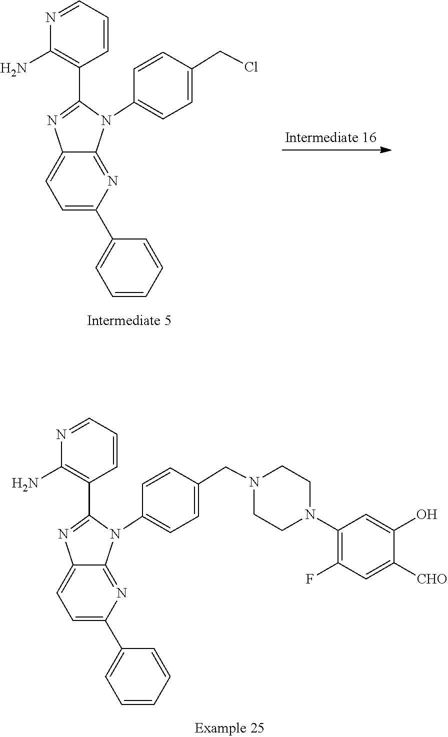 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3cc(O)c(C=O)cc3F)CC2)cc1]

To a solution of Intermediate 5 (150 mg, 334 μmol, HCl) and Intermediate 16 (104.66 mg, 401 μmol, HCl) in DMF (1.5 mL) were added NaI (25.1 mg, 167 μmol) and K₂CO₃ (231 mg, 1.67 mmol). The mixture was stirred at 80 °C for 2 hr. The reaction mixture was filtered. After purified by prep-HPLC (column: [water (NH₄HCO₃)-ACN]; B %: 51%-81%, 18 min), 4-(4-(4-(2-(2-aminopyridin-3-yl)-5-phenyl-3H-imidazo[4,5-b]pyridin-3-yl)benzyl)piperazin-1-yl)-5-fluoro-2-hydroxybenzaldehyde (Example 25, 16 mg, yield: 7.9% for two steps) was obtained as a yellow solid. MS: m/z=600.4 [M+H]+. ¹H NMR (400 MHz, Dimethylsulfoxide-d₆) S 10.88-10.62 (m, 1H), 10.03-9.88 (m, 1H), 8.32-8.22 (m, 1H), 8.08-7.94 (m, 4H), 7.56-7.44 (m, 6H), 7.43-7.29 (m, 2H), 7.15 (d, J=6.0 Hz, 1H), 7.02 (br s, 2H), 6.52-6.32 (m, 2H), 3.65 (br s, 2H), 3.25-3.20 (m, 4H), 2.58-3.56 (m, 4H). ¹H NMR (400 MHz, Dimethylsulfoxide+D₂O) δ 9.92 (s, 1H), 8.26 (d, J=8.4 Hz, 1H), 8.04-7.94 (m, 4H), 7.52-7.37 (m, 7H), 7.32 (d, J=13.6 Hz, 1H), 7.16 (dd, J=7.6, 1.6 Hz, 1H), 6.45 (d, J=7.6 Hz, 1H), 6.39 (dd, J=8.0, 5.2 Hz, 1H), 3.63 (s, 2H), 3.25-3.20 (m, 4H), 2.58-2.53 (br s, 4H). 19F NMR (400 MHz, Dimethylsulfoxide-d₆) δ −132.321.

### Example 26: 4-(4-(4-(2-(2-Aminopyridin-3-yl)-5-phenyl-3H-imidazo[4,5-b]pyridin-3-yl)benzyl)piperazin-1-yl)-3-fluoro-2-hydroxybenzaldehyde

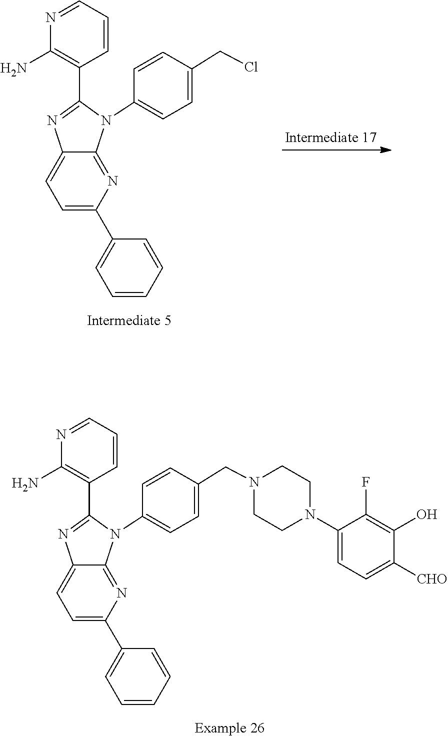 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3ccc(C=O)c(O)c3F)CC2)cc1]

To a solution of Intermediate 5 (150 mg, 334 μmol, HCl) and Intermediate 17 (104 mg, 401 μmol, HCl) in DMF (1.5 mL) were added NaI (25.1 mg, 167 μmol) and K₂CO₃ (231 mg, 1.67 mmol). The mixture was stirred at 80 °C for 2 hr. The reaction mixture was filtered. After purified by prep-HPLC (column: [water (NH₄HCO₃)-ACN]; B %: 47%-77%, 18 min), 4-(4-(4-(2-(2-aminopyridin-3-yl)-5-phenyl-3H-imidazo[4,5-b]pyridin-3-yl)benzyl)piperazin-1-yl)-3-fluoro-2-hydroxybenzaldehyde (Example 26, 16 mg, yield: 7.8% for two steps) was obtained as a yellow solid. MS: m/z=600.4 [M+H]⁺. ¹H NMR (400 MHz, Methanol-d₄) δ 9.81 (s, 1H), 8.20 (d, J=8.4 Hz, 1H), 8.04 (d, J=7.2 Hz, 2H), 7.98 (dd, J=4.8, 1.6 Hz, 1H), 7.95 (d, J=8.4 Hz, 1H), 7.58 (d, J=8.4 Hz, 2H), 7.49-7.32 (m, 7H), 6.66-6.61 (m, 1H), 6.48 (dd, J=7.6, 5.2 Hz, 1H), 3.71 (s, 2H), 3.40-3.36 (m, 4H), 2.72-2.66 (m, 4H). ¹⁹F NMR (400 MHz, Methanol-d₄) δ −151.272.

### Example 27: 4-(4-(4-(2-(2-Aminopyridin-3-yl)-5-phenyl-3H-imidazo[4,5-b]pyridin-3-yl)benzyl)piperazin-1-yl)-5-bromo-2-hydroxybenzaldehyde

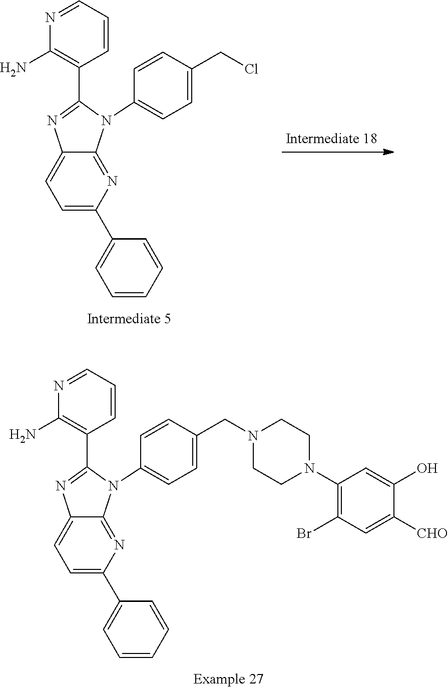 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3cc(O)c(C=O)cc3Br)CC2)cc1]

To a solution of Intermediate 5 (100 mg, 242 μmol) and Intermediate 18 (93.7 mg, 291 μmol, HCl salt) in DMF (1 mL) were added NaI (18.2 mg, 121 μmol) and K₂CO₃ (167 mg, 1.21 mmol). The mixture was stirred at 80 °C for 2 hr. The reaction mixture was filtered. After purified by prep-HPLC (column: [water (NH₄HCO₃)-ACN]; B %: 57%-87%, 18 min), 4-(4-(4-(2-(2-aminopyridin-3-yl)-5-phenyl-3H-imidazo[4,5-b]pyridin-3-yl)benzyl)piperazin-1-yl)-5-bromo-2-hydroxybenzaldehyde (Example 27, 24.2 mg, yield: 14% for two steps) was obtained as a yellow solid. MS: m/z=660.3 [M+H]⁺. ¹H NMR (400 MHz, Methanol-d₄) δ 9.82 (s, 1H), 8.19 (d, J=8.4 Hz, 1H), 8.04 (d, J=7.6 Hz, 2H), 7.98 (d, J=4.8 Hz, 1H), 7.94 (d, J=8.8 Hz, 1H), 7.85-7.82 (m, 1H), 7.58 (d, J=8.0 Hz, 2H), 7.49-7.40 (m, 4H), 7.39-7.32 (m, 2H), 6.62-6.59 (m, 1H), 6.51-6.42 (m, 1H), 3.74 (s, 2H), 3.28-3.16 (m, 4H), 2.81-2.68 (m, 4H).

### Example 28: 4-(4-(4-(2-(2-Aminopyridin-3-yl)-5-phenyl-3H-imidazo[4,5-b]pyridin-3-yl)benzyl)piperazin-1-yl)-2-hydroxy-5-vinylbenzaldehyde

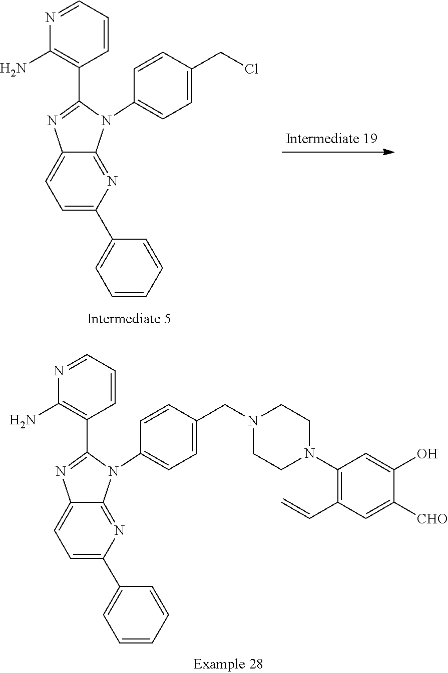 [SMILES: Nc1ncccc1-c1nc2ccc(-c3ccccc3)nc2n1-c1ccc(CCl)cc1 ; C=Cc1cc(C=O)c(O)cc1N1CCN(Cc2ccc(-n3c(-c4cccnc4N)nc4ccc(-c5ccccc5)nc43)cc2)CC1]

To a solution of Intermediate 5 (100 mg, 242 μmol) and Intermediate 19 (78.3 mg, 291 μmol, HCl) in DMF (1 mL) were added NaI (18.2 mg, 121 μmo) and K₂CO₃ (167 mg, 1.21 mmol). The mixture was stirred at 80 °C for 2 hr. The reaction mixture was filtered. After purified by prep-HPLC (column: [water (NH₄HCO₃)-ACN]; B %: 62%-92%, 10 min), 4-(4-(4-(2-(2-aminopyridin-3-yl)-5-phenyl-3H-imidazo[4,5-b]pyridin-3-yl)benzyl)piperazin-1-yl)-2-hydroxy-5-vinylbenzaldehyde (Example 28, 8.9 mg, yield: 5.9% for two steps) was obtained as a yellow solid. MS: m/z=608.0 [M+H]+. ¹H NMR (400 MHz, Methanol-d₄) δ 9.84 (s, 1H), 8.20 (d, J=8.4 Hz, 1H), 8.04 (d, J=7.2 Hz, 2H), 8.00-7.93 (m, 2H), 7.75-7.69 (m, 1H), 7.62-7.54 (m, 2H), 7.49-7.33 (m, 6H), 6.87-6.71 (m, 1H), 6.52-6.45 (m, 2H), 5.75-5.65 (m, 1H), 5.22 (d, J=12.4 Hz, 1H), 3.72 (s, 2H), 3.16-3.13 (m, 4H), 2.75-7.67 (m, 4H).

### Example 29: 5-(4-(4-(2-(2-Aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde

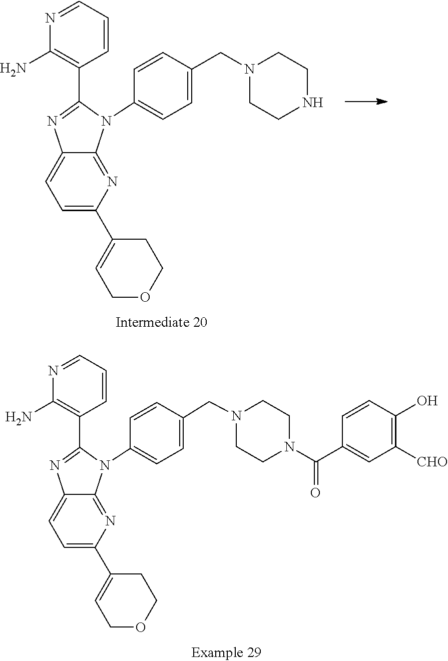 [SMILES: Nc1ncccc1-c1nc2ccc(C3=CCOCC3)nc2n1-c1ccc(CN2CCNCC2)cc1 ; Nc1ncccc1-c1nc2ccc(C3=CCOCC3)nc2n1-c1ccc(CN2CCN(C(=O)c3ccc(O)c(C=O)c3)CC2)cc1]

To a solution of Intermediate 20 (100 mg, 214 μmol) in CH₂Cl₂ (0.5 mL) were added DIEA (82.9 mg, 641 μmol), 3-formyl-4-hydroxybenzoic acid (35.5 mg, 214 μmol) and HATU (81.3 mg, 214 μmol). The mixture was stirred at 25 °C for 1 hr. The mixture was diluted with H₂O (10 mL) and extracted with CH₂Cl₂ (10 mL×2). The combined organic layers were washed with brine (10 mL), dried over anhydrous Na₂SO₄, filtered and concentrated. After purified by prep-HPLC (column: Welch Xtimate C18 150×25 mm×5 □m: mobile phase: [water (NH₃H₂O+NH₄HCO₃)-ACN]; gradient: 25%-55% B over 7 min), 5-(4-(4-(2-(2-aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde (Example 29, 8.0 mg, yield: 6.1%) was obtained as a yellow solid. MS: m/z=616.3 [M+H]⁺. ¹H NMR (400 MHz, Dimethylsulfoxide-d_{f};) δ 10.27 (s, 1H), 8.16 (d, J=8.4 Hz, 1H), 7.99-7.95 (m, 1H), 7.67 (d, J=2.0 Hz, 1H), 7.60-7.52 (m, 2H), 7.47-7.44 (m, 2H), 7.41-7.35 (m, 2H), 7.13-7.08 (m, 1H), 7.05-6.98 (m, 3H), 6.68 (s, 1H), 6.39-6.34 (m, 1H), 4.28-4.21 (m, 2H), 3.79 (t, J=5.2 Hz, 2H), 3.61 (s, 2H), 3.57-3.47 (m, 6H), 2.47-2.37 (m, 4H).

### Example 30: 4-(4-(4-(2-(2-Aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde

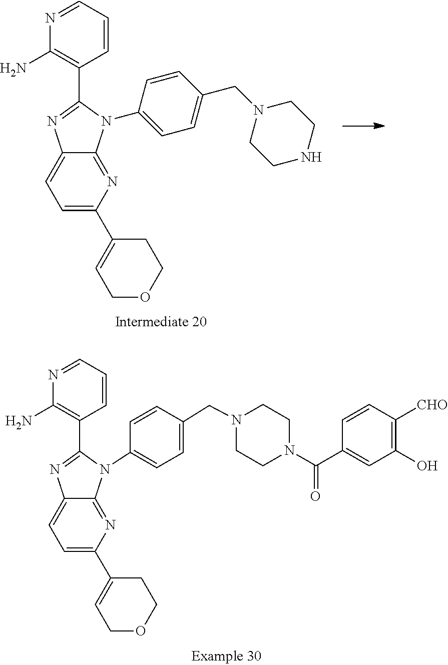 [SMILES: Nc1ncccc1-c1nc2ccc(C3=CCOCC3)nc2n1-c1ccc(CN2CCNCC2)cc1 ; Nc1ncccc1-c1nc2ccc(C3=CCOCC3)nc2n1-c1ccc(CN2CCN(C(=O)c3ccc(C=O)c(O)c3)CC2)cc1]

Following the general procedures described in Example 29, the reactions was carried out using Intermediate 20. After purified by prep-HPLC (column: Welch Xtimate C18 150×25 mm×5 μm; mobile phase: [water (NH₃H₂O+NH₄HCO₃)-ACN]; gradient: 30%-60% B over 7 min), 4-(4-(4-(2-(2-aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde (Example 30, 18.3 mg, yield: 7.7%) was obtained as a yellow solid. MS: m/z=616.3 [M+H]⁺. ¹H NMR (400 MHz, Dimethylsulfoxide-d₆) δ 11.32-10.62 (m, 1H), 10.27 (s, 1H), 8.15 (d, J=8.0 Hz, 1H), 7.97 (d, J=3.2 Hz, 1H), 7.69 (d, J=7.6 Hz, 1H), 7.58 (d, J=8.4 Hz, 1H), 7.45 (d, J=8.4 Hz, 2H), 7.37 (d, J=8.4 Hz, 2H), 7.10 (dd, J=7.6, 2.0 Hz, 1H), 7.02-6.90 (m, 4H), 6.67 (s, 1H), 6.36 (dd, J=8.0, 4.8 Hz, 1H), 4.28-4.21 (m, 2H), 3.78-3.76 (m, 2H), 3.68-3.59 (m, 4H), 3.35-3.26 (m, 6H), 2.39-2.31 (m, 2H).

### Example 31: 4-(4-(4-(2-(2-Aminopyridin-3-yl)-5-morpholino-3H-imidazo[4,5-b]pyridin-3-yl)benzyl)piperazin-1-yl)-2-hydroxybenzaldehyde

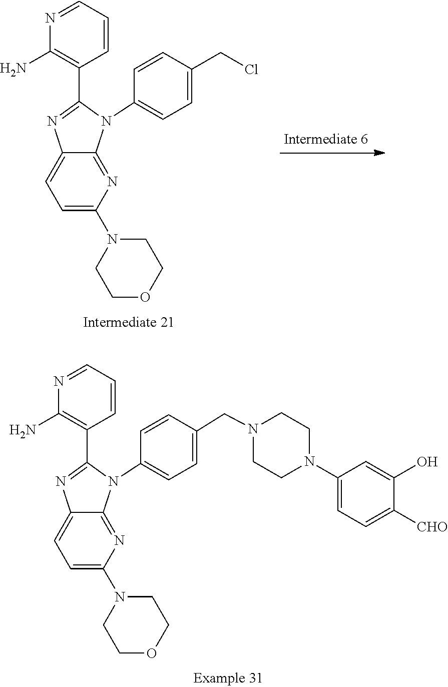 [SMILES: Nc1ncccc1-c1nc2ccc(N3CCOCC3)nc2n1-c1ccc(CCl)cc1 ; Nc1ncccc1-c1nc2ccc(N3CCOCC3)nc2n1-c1ccc(CN2CCN(c3ccc(C=O)c(O)c3)CC2)cc1]

To a mixture of Intermediate 21 (150 mg, 356 μmol) and Intermediate 6 (73.5 mg, 356 μmol) in DMF (3 mL) were added NaI (16.0 mg, 106 μmol) and K₂CO₃ (246 mg, 1.78 mmol). The mixture was stirred at 80 °C for 1 hr. The reaction mixture was diluted with H₂O (1 mL), filtered, and concentrated under reduced pressure. After purified by silica gel flash chromatography (Eluent of 1%-7% MeOH in CH₂Cl₂) and prep-HPLC (column: Waters xbridge 150×25 mm 10 μm; mobile phase: [water (NH₄HCO₃)-ACN]; gradient: 39%-69% B over 10 min), 4-(4-(4-(2-(2-aminopyridin-3-yl)-5-morpholino-3H-imidazo[4,5-b]pyridin-3-yl)benzyl)piperazin-1-yl)-2-hydroxybenzaldehyde (Example 31, 9.5 mg, yield: 4.2%) was obtained as a yellow powder. MS: m/z=591.3 [M+H]⁺. ¹H NMR (400 MHz, Dimethylsulfoxide-d₆) δ 11.10 (br s, 1H), 9.73 (s, 1H), 7.98 (d, J=9.2 Hz, 1H), 7.92 (dd, J=4.8, 1.6 Hz, 1H), 7.50-7.44 (m, 3H), 7.33 (d, J=8.0 Hz, 2H), 7.03 (br s, 2H), 6.98 (dd, J=7.6, 2.0 Hz, 1H), 6.90 (d, J=8.8 Hz, 1H), 6.60 (dd, J=8.8, 2.0 Hz, 1H), 6.33-6.26 (m, 2H), 3.70-3.66 (m, 4H), 3.61 (s, 2H), 3.43-3.38 (m, 8H), 3.34-3.33 (m, 4H).

### Example 32: 5-(4-(4-(2-(2-Aminopyridin-3-yl)-5-morpholino-3H-imidazo[4,5-b]pyridin-3-yl)benzyl)piperazine-1-carbonyl)-2-hydroxybenzaldehyde

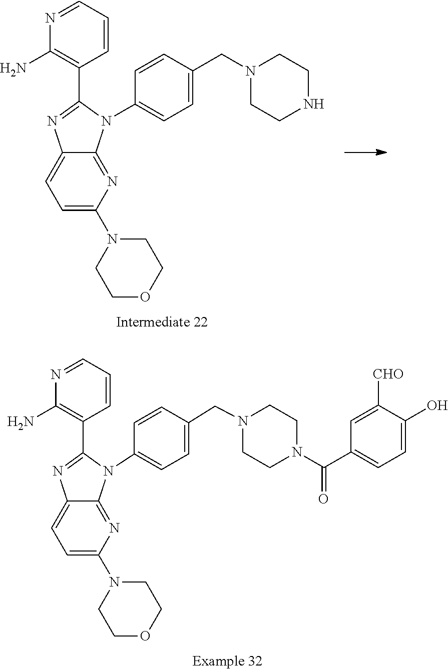 [SMILES: Nc1ncccc1-c1nc2ccc(N3CCOCC3)nc2n1-c1ccc(CN2CCNCC2)cc1 ; Nc1ncccc1-c1nc2ccc(N3CCOCC3)nc2n1-c1ccc(CN2CCN(C(=O)c3ccc(O)c(C=O)c3)CC2)cc1]

A mixture of Intermediate 22 (30 mg, 59.2 μmol, HCl salt), 3-formyl-4-hydroxybenzoic acid (10.8 mg, 65.1 μmol), HOBt (12 mg, 88.8 μmol), EDCI (17 mg, 88.8 μmol) and DIEA (38.2 mg, 296 μmol) in DMF (2 mL) was stirred at 30 °C for 16 hr. The mixture was filtered and concentrated under reduced pressure. After purified by prep-HPLC (column: Waters xbridge 150*25 mm*10 μm; mobile phase: [water (NH₄HCO₃)-ACN]; B %: 19%-49%, over 10 min), 5-(4-(4-(2-(2-aminopyridin-3-yl)-5-morpholino-3H-imidazo[4,5-b]pyridin-3-yl)benzyl)piperazine-1-carbonyl)-2-hydroxybenzaldehyde (Example 32, 9.3 mg, yield: 24%) was obtained as a yellow lyophilized powder. MS: m/z=619.2 [M+H]⁺. ¹H NMR (400 MHz, Dimethylsulfoxide-d₆) δ 10.27-10.24 (m, 1H), 7.98 (d, J=8.8 Hz, 1H), 7.92 (dd, J=4.8, 1.6 Hz, 1H), 7.66 (s, 1H), 7.53 (d, J=8.0 Hz, 1H), 7.44 (d, J=8.0 Hz, 2H), 7.32 (d, J=8.4 Hz, 2H), 7.02 (br s, 2H), 6.98 (dd, J=7.6, 2.0 Hz, 2H), 6.89 (d, J=8.8 Hz, 1H), 6.31 (dd, J=7.6, 4.8 Hz, 1H), 3.69-3.65 (m, 4H), 3.60 (s, 2H), 3.56-3.49 (m, 4H), 3.41-3.38 (m, 4H), 2.45-2.39 (m, 4H).

### Example 33: 4-(4-(4-(2-(2-Aminopyridin-3-yl)-5-morpholino-3H-imidazo[4,5-b]pyridin-3-yl)benzyl)piperazine-1-carbonyl)-2-hydroxybenzaldehyde

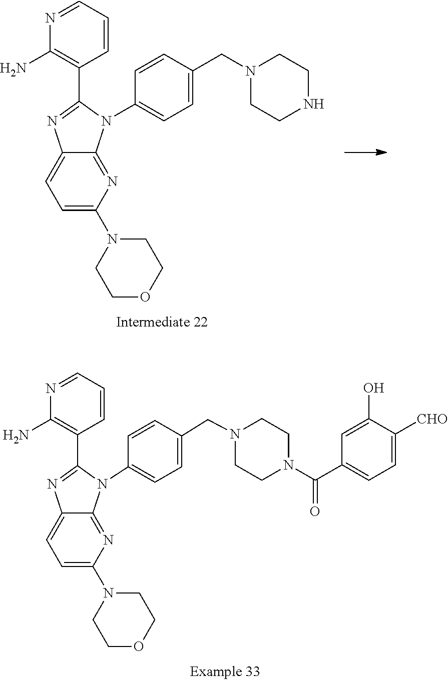 [SMILES: Nc1ncccc1-c1nc2ccc(N3CCOCC3)nc2n1-c1ccc(CN2CCNCC2)cc1 ; Nc1ncccc1-c1nc2ccc(N3CCOCC3)nc2n1-c1ccc(CN2CCN(C(=O)c3ccc(C=O)c(O)c3)CC2)cc1]

Following the general procedures described in Example 32, the reaction was carried out using Intermediate 22. After purified by prep-HPLC (column: Phenomenex C18 150*25 mm*10 μm; mobile phase: [water (NH₄HCO₃)-ACN]; B %: 23%-53%, over 10 min), 4-(4-(4-(2-(2-aminopyridin-3-yl)-5-morpholino-3H-imidazo[4,5-b]pyridin-3-yl)benzyl)piperazine-1-carbonyl)-2-hydroxybenzaldehyde (Example 33, 9.9 mg, yield: 25%) was obtained as a yellow lyophilized powder. MS: m/z=619.2 [M+H]+. ¹H NMR (400 MHz, Dimethylsulfoxide-d₆) δ 11.12-10.81 (m, 1H), 10.29 (s, 1H), 7.98 (d, J=8.8 Hz, 1H), 7.92 (dd, J=4.8, 1.6 Hz, 1H), 7.69 (d, J=8.0 Hz, 1H), 7.44 (d, J=8.4 Hz, 2H), 7.32 (d, J=8.4 Hz, 2H), 7.01 (br s, 2H), 6.98 (dd, J=7.6, 1.6 Hz, 1H), 6.94 (s, 1H), 6.89 (d, J=8.8 Hz, 2H), 6.31 (dd, J=7.6, 4.8 Hz, 1H), 3.86-3.60 (m, 8H), 3.60 (s, 2H), 3.41-3.38 (m, 4H), 2.48-2.31 (m, 4H).

### Example 34: 3-(2-(2-Aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile

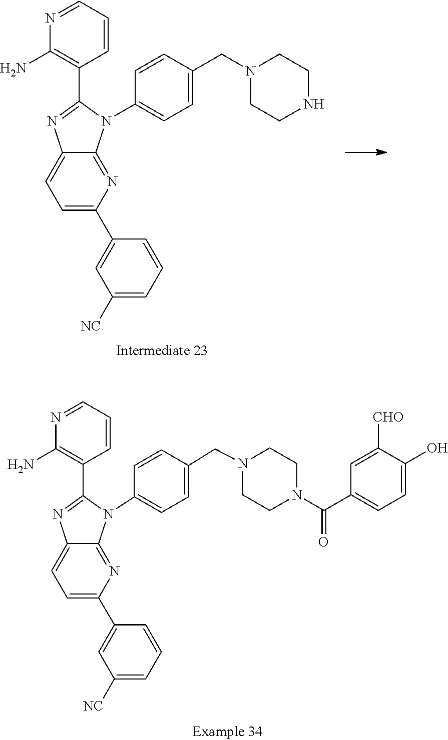 [SMILES: N#Cc1cccc(-c2ccc3nc(-c4cccnc4N)n(-c4ccc(CN5CCNCC5)cc4)c3n2)c1 ; N#Cc1cccc(-c2ccc3nc(-c4cccnc4N)n(-c4ccc(CN5CCN(C(=O)c6ccc(O)c(C=O)c6)CC5)cc4)c3n2)c1]

To a solution of Intermediate 23 (40 mg, 82.2 μmol), 3-formyl-4-hydroxybenzoic acid (13.7 mg, 82.2 μmol) in DMF (1 mL) were added HOBt (16.7 mg, 123 μmol), EDCI (23.6 mg, 123 μmol) and DIEA (42.5 mg, 329 μmol). The mixture was stirred at 25 °C for 16 hr. The reaction mixture was filtered and concentrated under reduced pressure. After purified by prep-HPLC (column: Phenomenex C₁₈ 150×25 mm×10 μm; mobile phase: [water(NH₄HCO₃)-ACN]; gradient: 25%-55% B over 14 min), 3-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile (Example 34, 2.5 mg, yield: 4.7%) was obtained as a yellow solid. MS: m/z=635.4 [M+H]+. ¹H NMR (400 MHz, Methanol-d₄) δ 10.11 (s, 1H), 8.40-8.34 (m, 2H), 8.24 (d, J=8.4 Hz, 1H), 8.04-7.98 (m, 2H), 7.82 (d, J=2.0 Hz, 1H), 7.74-7.71 (m, 1H), 7.64-7.57 (m, 4H), 7.48-7.45 (m, 2H), 7.36-7.33 (m, 1H), 7.04 (d, J=8.0 Hz, 1H), 6.50-6.46 (m, 1H), 4.59 (s, 2H), 3.73-3.67 (m, 4H), 2.63-2.51 (m, 4H).

### Example 35: 3-(2-(2-Aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile

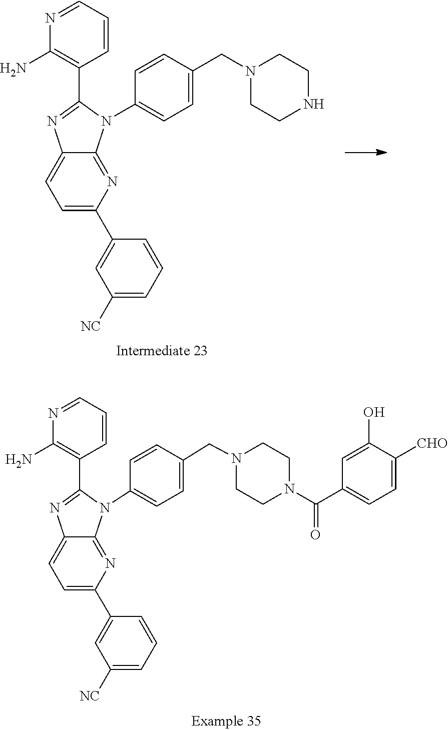 [SMILES: N#Cc1cccc(-c2ccc3nc(-c4cccnc4N)n(-c4ccc(CN5CCNCC5)cc4)c3n2)c1 ; N#Cc1cccc(-c2ccc3nc(-c4cccnc4N)n(-c4ccc(CN5CCN(C(=O)c6ccc(C=O)c(O)c6)CC5)cc4)c3n2)c1]

Following the general procedures described in Example 34, the reaction was carried out using corresponding starting material 4-formyl-3-hydroxybenzoic acid. After purified by prep-HPLC (column: Phenomenex Cis 150×25 mm×10 μm; mobile phase: [water(NH₄HCO₃)-ACN]; gradient: 28%-58% B over 14 min), 3-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile (Example 35, 14.9 mg, yield: 18%) was obtained as a yellow lyophilized powder. MS: m/z=635.4 [M+H]⁺. ¹H NMR (400 MHz, Methanol-d₄) δ 10.11 (s, 1H), 8.40-8.34 (m, 2H), 8.24 (d, J=8.4 Hz, 1H), 8.04-7.98 (m, 2H), 7.80 (d, J=8.0 Hz, 1H), 7.74-7.71 (m, 1H), 7.64-7.61 (m, 1H), 7.59-7.56 (m, 2H), 7.48-7.44 (m, 2H), 7.35-7.32 (m, 1H), 7.04-7.00 (m, 1H), 6.97 (s, 1H), 6.47 (dd, J=7.6, 4.8 Hz, 1H), 3.82-3.80 (m, 2H), 3.69 (s, 2H), 3.46-3.44 (m, 2H), 2.65-2.47 (m, 4H).

### Example 36: 2-(2-(2-Aminopyridin-3-yl)-3-(4-((4-(3-formyl-4-hydroxybenzoyl piperazin-1-yl)methyl)phenyl)-3H-imidazo[4,5-b]pyridin-5-yl)benzonitrile

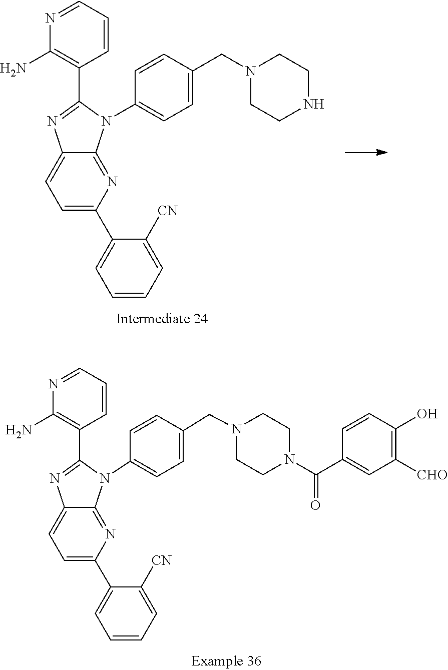 [SMILES: N#Cc1ccccc1-c1ccc2nc(-c3cccnc3N)n(-c3ccc(CN4CCNCC4)cc3)c2n1 ; N#Cc1ccccc1-c1ccc2nc(-c3cccnc3N)n(-c3ccc(CN4CCN(C(=O)c5ccc(O)c(C=O)c5)CC4)cc3)c2n1]

To a solution of Intermediate 24 (50 mg, 103 μmol) and 3-formyl-4-hydroxybenzoic acid (17 mg, 103 μmol) in DMF (1 mL) were added EDCI (30 mg, 154 μmol), DIEA (66 mg, 514 μmol) and HOBt (21 mg, 154 μmol). The mixture was stirred at 25 °C for 16 hr. The resulting mixture was filtered and concentrated under reduced pressure. After purified by prep-HPLC (column: Phenomenex C18 150×25 mm×10 μm; mobile phase: [water(NH₄HCO₃)-ACN]; gradient: 29%-59% B over 12 min), 2-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile (Example 36, 9 mg, yield: 14%) was obtained as a yellow powder. MS: m/z=635.4 [M+H]⁺. 1H NMR (400 MHz, Methanol-d₄) 10.10 (s, 1H), 8.30 (d, J=8.4 Hz, 1H), 8.02-7.97 (m, 1H), 7.90-7.86 (m, 1H), 7.86-7.80 (m, 3H), 7.77-7.71 (m, 1H), 7.63-7.56 (m, 2H), 7.52-7.51 (m, 4H), 7.39-7.29 (m, 1H), 7.03 (d, 1=8.8 Hz, 1H), 6.51-6.46 (m, 1H), 3.71-3.62 (m, 6H), 2.60-2.46 (m, 4H).

### Example 37: 2-(2-(2-Aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile

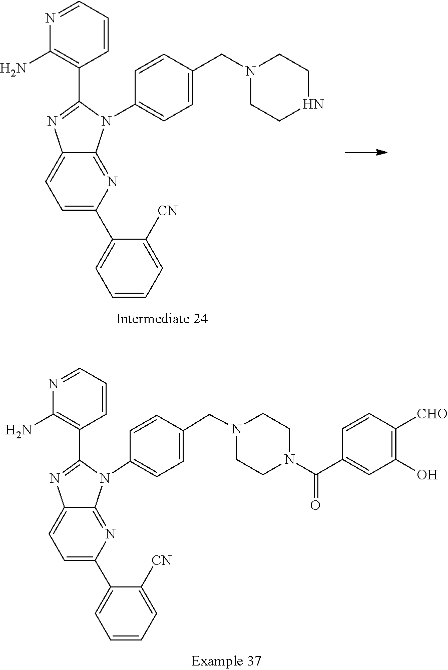 [SMILES: N#Cc1ccccc1-c1ccc2nc(-c3cccnc3N)n(-c3ccc(CN4CCNCC4)cc3)c2n1 ; N#Cc1ccccc1-c1ccc2nc(-c3cccnc3N)n(-c3ccc(CN4CCN(C(=O)c5ccc(C=O)c(O)c5)CC4)cc3)c2n1]

Following the general procedures described in Example 36, the reaction was carried out using 4-formyl-3-hydroxybenzoic acid and Intermediate 24. After purified by prep-HPLC (column: Phenomenex C18 150×25 mm×10 μm; mobile phase: [water(NH₄HCO₃)-ACN]; gradient: 32%-62% B over 10 min), 2-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile (Example 37, 13.2 mg, yield: 20%) was obtained as a yellow powder. MS: m/z=635.3 [M+H]+. ¹H NMR (400 MHz, Methanol-d₄) δ 10.11 (s, 1H), 8.30 (d, J=8.4 Hz, 1H), 8.00 (dd, J=5.2, 1.6 Hz, 1H), 7.90-7.71 (m, 5H), 7.59-7.54 (m, 1H), 7.51-7.50 (m, 4H), 7.35 (dd, J=7.6, 1.6 Hz, 1H), 7.00 (dd, J=8.0, 1.2 Hz, 1H), 6.96-6.94 (m, 1H), 6.49 (dd, J=7.6, 5.2 Hz, 1H), 3.84-3.74 (m, 2H), 3.66 (s, 2H), 3.45-3.36 (m, 2H), 2.63-2.58 (m, 2H), 2.50-2.46 (m, 2H).

### Example 38: 4-(2-(2-Aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile

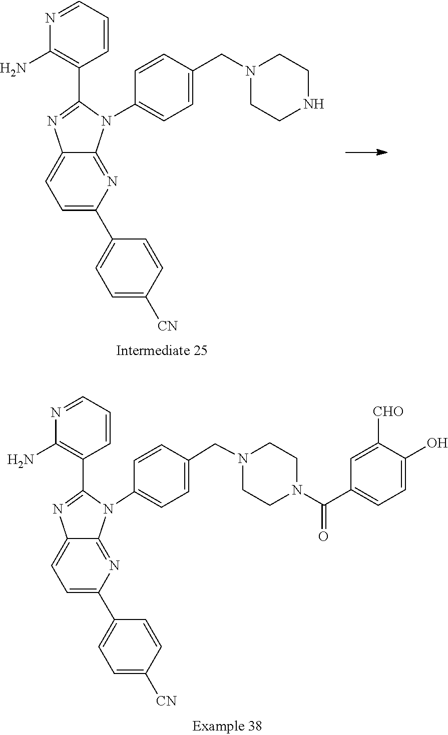 [SMILES: N#Cc1ccc(-c2ccc3nc(-c4cccnc4N)n(-c4ccc(CN5CCNCC5)cc4)c3n2)cc1 ; N#Cc1ccc(-c2ccc3nc(-c4cccnc4N)n(-c4ccc(CN5CCN(C(=O)c6ccc(O)c(C=O)c6)CC5)cc4)c3n2)cc1]

Following the general procedures described in Example 36, the reaction was carried out using 3-formyl-4-hydroxybenzoic acid and Intermediate 25. After purified by prep-HPLC (column: Phenomenex Cis 150×25 mm×10 μm; mobile phase: [water(NH₄HCO₃)-ACN]; gradient: 30%-50% B over 14 min), 4-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile (Example 38, 24.3 mg, yield: 30%) was obtained as a yellow lyophilized powder. MS: m/z=635.4 [M+H]⁺. ¹H NMR (400 MHz, Methanol-d₄) δ 10.13 (s, 1H), 8.26-8.22 (m, 3H), 8.06-8.03 (m, 1H), 8.00-7.97 (m, 1H), 7.83-7.77 (m, 3H), 7.64-7.60 (m, 1H), 7.58-7.55 (m, 2H), 7.47-7.44 (m, 2H), 7.34-7.31 (m, 1H), 7.03 (d, J=9.2 Hz, 1H), 6.50-6.45 (m, 1H), 3.73-3.65 (m, 4H), 3.19-3.14 (m, 2H), 2.59-2.56 (m, 4H).

### Example 39: 4-(2-(2-Aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile

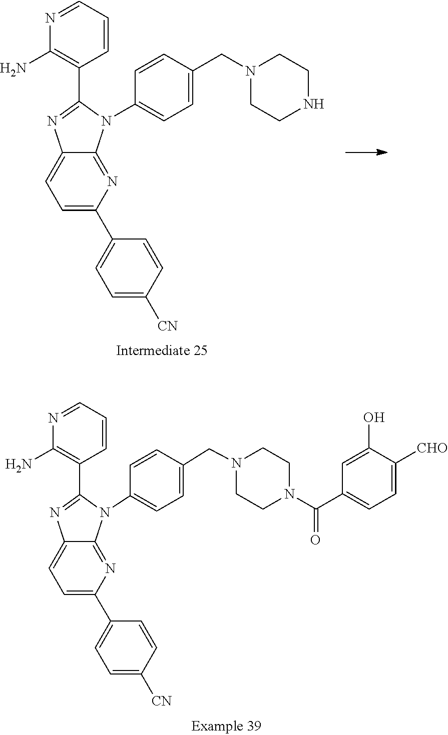 [SMILES: N#Cc1ccc(-c2ccc3nc(-c4cccnc4N)n(-c4ccc(CN5CCNCC5)cc4)c3n2)cc1 ; N#Cc1ccc(-c2ccc3nc(-c4cccnc4N)n(-c4ccc(CN5CCN(C(=O)c6ccc(C=O)c(O)c6)CC5)cc4)c3n2)cc1]

Following the general procedures described in Example 36, the reaction was carried out using 4-formyl-3-hydroxybenzoic acid and Intermediate 25. After purified by prep-HPLC (column: Phenomenex C₁₈ 150×25 mm×10 μm; mobile phase: [water(NH₄HCO₃)-ACN]; gradient: 32%-52% B over 14 min), 4-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile (Example 39, 12.4 mg, yield: 16%) was obtained as a yellow lyophilized powder. MS: m/z=635.4 [M+H]⁺. ¹H NMR (400 MHz, Methanol-d₄) δ 10.11 (s, 1H), 8.40-8.24 (m, 3H), 8.04-7.98 (m, 2H), 7.80-7.61 (m, 3H), 7.59-7.56 (m, 2H), 7.48-7.44 (m, 2H), 7.35-7.32 (m, 1H), 7.04-7.00 (m, 1H), 6.97 (s, 1H), 6.47 (dd, J=7.6, 4.8 Hz, 1H), 3.82-3.80 (m, 2H), 3.69 (s, 2H), 3.46-3.44 (m, 2H), 2.65-2.47 (m, 4H).

### Example 40: 5-(4-(4-(2-(2-Aminopyridin-3-yl)-5-(3-fluorophenyl)-3H-imidazo[4,5-b]pyridin-3-yl)benzyl)piperazine-1-carbonyl)-2-hydroxybenzaldehyde

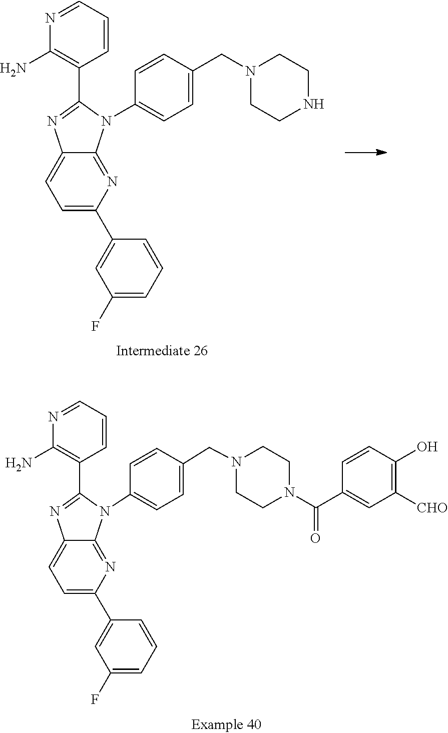 [SMILES: Nc1ncccc1-c1nc2ccc(-c3cccc(F)c3)nc2n1-c1ccc(CN2CCNCC2)cc1 ; Nc1ncccc1-c1nc2ccc(-c3cccc(F)c3)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 26. After purified by prep-HPLC (column: Phenomenex C18 150×25 mm×10 μm; mobile phase: [water(NH₄HCO₃)-ACN]; gradient: 35%-55% B over 14 min), 5-(4-(4-(2-(2-aminopyridin-3-yl)-5-(3-fluorophenyl)-3H-imidazo[4,5-b]pyridin-3-yl)benzyl)piperazine-1-carbonyl)-2-hydroxybenzaldehyde (Example 40, 17.7 mg, yield: 14%) was obtained as a yellow powder. MS: m/z=628.2 [M+H]¹H NMR (400 MHz, Dimethysulfoxide-d₆) δ 10.27 (s, 1H), 8.29 (d, J=8.4 Hz, 1H), 8.05 (d, J=8.4 Hz, 1H), 8.00 (dd, J=4.8, 1.6 Hz, 1H), 7.90 (d, J=8.0 Hz, 1H), 7.84-7.79 (m, 1H), 7.67 (d, J=2.0 Hz, 1H), 7.60-7.41 (m, 7H), 7.25-7.20 (m, 1H), 7.16 (dd, J=7.6, 1.6 Hz, 1H), 7.03-7.00 (m, 2H), 6.39 (dd, J=7.6, 4.8 Hz, 1H), 3.64 (s, 2H), 3.59-3.49 (m, 4H), 2.46-2.43 (m, 4H). ¹⁹F NMR (400 MHz, Dimethylsulfoxide-d₆) δ −112.913.

### Example 41: 4-(4-(4-(2-(2-Aminopyridin-3-yl)-5-(3-fluorophenyl)-3H-imidazo[4,5-b]pyridin-3-yl)benzyl)piperazine-1-carbonyl)-2-hydroxybenzaldehyde

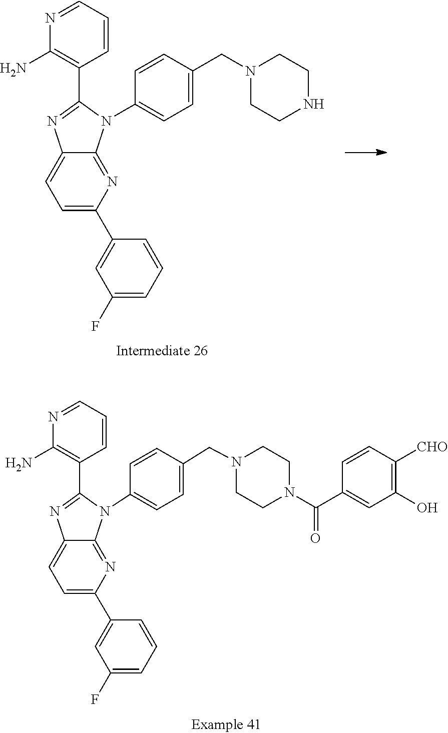 [SMILES: Nc1ncccc1-c1nc2ccc(-c3cccc(F)c3)nc2n1-c1ccc(CN2CCNCC2)cc1 ; Nc1ncccc1-c1nc2ccc(-c3cccc(F)c3)nc2n1-c1ccc(CN2CCN(C(=O)c3ccc(C=O)c(O)c3)CC2)cc1]

Following the general procedures described in Example 36, the reaction was carried out using 4-formyl-3-hydroxybenzoic acid and Intermediate 26. After purified by prep-HPLC (column: Phenomenex C18 150×25 mm×10 μm; mobile phase: [water(NH₄HCO₃)-ACN]; gradient: 35%-55% B over 14 min), 4-(4-(4-(2-(2-aminopyridin-3-yl)-5-(3-fluorophenyl)-3H-imidazo[4,5-b]pyridin-3-yl)benzyl)piperazine-1-carbonyl)-2-hydroxybenzaldehyde (Example 41, 33.2 mg, yield: 28%) was obtained as a yellow powder. MS: m/z=628.2 [M+H]⁺. ¹H NMR (400 MHz, Dimethysulfoxide-d₆) δ 11.23-10.73 (m, 1H), 10.29 (s, 1H), 8.29 (d, J=8.4 Hz, 1H), 8.05 (d, J=8.4 Hz, 1H), 8.00 (dd, J=4.8, 1.6 Hz, 1H), 7.90 (d, J=8.4 Hz, 1H), 7.84-7.79 (m, 1H), 7.70 (d, J=8.0 Hz, 1H), 7.52-7.45 (m, 5H), 7.25-7.21 (m, 1H), 7.16 (dd, J=7.6, 1.6 Hz, 1H), 7.06-6.98 (m, 2H), 6.97-6.94 (m, 1H), 6.91 (d, J=8.0 Hz, 1H), 6.39 (dd, J=7.6, 4.8 Hz, 1H), 3.69-3.61 (m, 4H), 3.29-3.27 (m, 2H), 2.48-2.37 (m, 4H). ¹⁹F NMR (400 MHz, Dimethylsulfoxide-d₆) δ −112.913.

### Example 42: 5-(4-(4-(2-(2-Aminopyridin-3-yl)-5-(3-chlorophenyl)-3H-imidazo[4,5-b]pyridin-3-yl)benzyl)piperazine-1-carbonyl)-2-hydroxybenzaldehyde

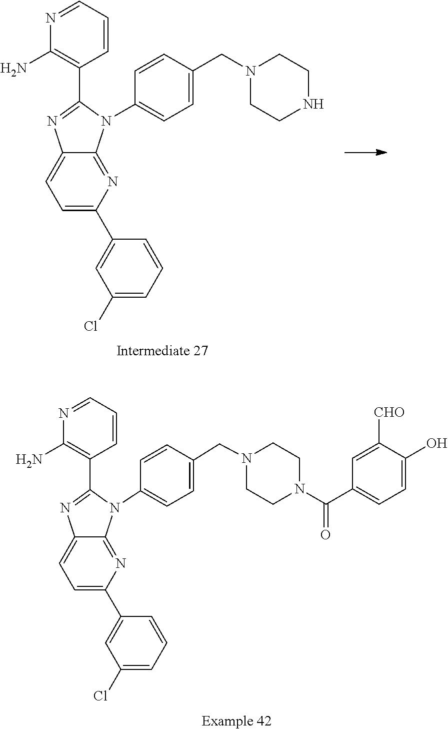 [SMILES: Nc1ncccc1-c1nc2ccc(-c3cccc(Cl)c3)nc2n1-c1ccc(CN2CCNCC2)cc1 ; Nc1ncccc1-c1nc2ccc(-c3cccc(Cl)c3)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 27. After purified by prep-HPLC (column: Waters xbridge 150×25 mm 10 μm; mobile phase: [water (NH₄HCO₃)-ACN]; gradient: 37%-67% B over 14 min), 5-(4-(4-(2-(2-aminopyridin-3-yl)-5-(3-chlorophenyl)-3H-imidazo[4,5-b]pyridin-3-yl)benzyl)piperazine-1-carbonyl)-2-hydroxybenzaldehyde (Example 42, 31.6 mg, yield: 23%) was obtained as a pink solid. MS: m/z=644.3, 646.3 [M+H]⁺. ¹H NMR (400 MHz, Methanol-d₄) δ 10.12 (s, 1H), 8.21 (d, J=8.4 Hz, 1H), 8.04 (s, 1H), 7.99-7.94 (m, 3H), 7.82 (d, J=2.0 Hz, 1H), 7.62-7.54 (m, 3H), 7.46 (d, J=8.4 Hz, 2H), 7.43-7.33 (m, 31H), 7.02 (d, J=8.8 Hz, 1H), 6.48 (dd, 1=7.6, 5.2 Hz, 1H), 3.75-3.60 (m, 6H), 2.61-2.51 (m, 4H).

### Example 43: 4-(4-(4-(2-(2-Aminopyridin-3-yl)-5-(3-chlorophenyl)-3H-imidazo[4,5-b]pyridin-3-yl)benzyl)piperazine-1-carbonyl)-2-hydroxybenzaldehyde

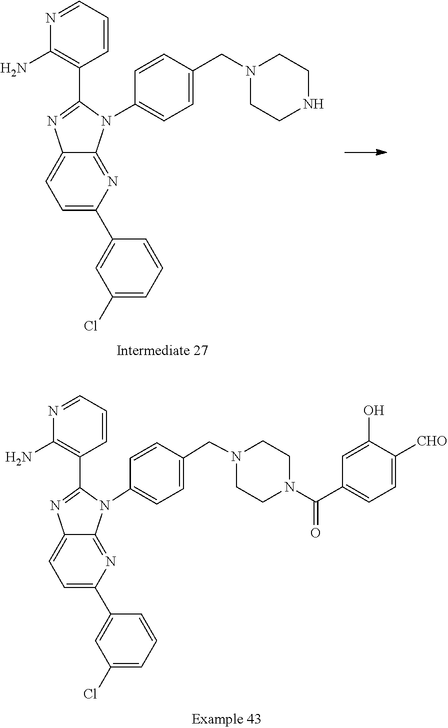 [SMILES: Nc1ncccc1-c1nc2ccc(-c3cccc(Cl)c3)nc2n1-c1ccc(CN2CCNCC2)cc1 ; Nc1ncccc1-c1nc2ccc(-c3cccc(Cl)c3)nc2n1-c1ccc(CN2CCN(C(=O)c3ccc(C=O)c(O)c3)CC2)cc1]

Following the general procedures described in Example 36, the reaction was carried out using 4-formyl-3-hydroxybenzoic acid and Intermediate 27. After purified by prep-HPLC (column: Waters xbridge 150*25 mm 10 μm; mobile phase: [water (NH₄HCO₃)-ACN]; gradient: 41%-71% B over 14 min), 4-(4-(4-(2-(2-aminopyridin-3-yl)-5-(3-chlorophenyl)-3H-imidazo[4,5-b]pyridin-3-yl)benzyl)piperazine-1-carbonyl)-2-hydroxybenzaldehyde (Example 43, 33.9 mg, yield: 26%) was obtained as a yellow lyophilized powder. MS: m/z=644.4, 646.4 [M+H]⁺. ¹H NMR (400 MHz, Methanol-d₄) δ 10.12 (s, 1H), 8.21 (d, J=8.4 Hz, 1H), 8.04 (s, 1H), 7.99-7.94 (m, 3H), 7.80 (d, J=7.6 Hz, 1H), 7.57 (d, J=8.4 Hz, 2H), 7.45 (d, J=8.4 Hz, 2H), 7.41 (d, J=7.6 Hz, 1H), 7.38-7.32 (m, 2H), 7.01 (d, J=8.0 Hz, 1H), 6.97 (s, 1H), 6.48 (dd, J=7.6, 4.8 Hz, 1H), 3.84-3.76 (m, 2H), 3.69 (s, 2H), 3.48-3.43 (m, 2H), 2.65-2.48 (m, 4H).

### Example 44: 5-(4-(4-(2-(2-Aminopyridin-3-yl)-5-(I-methyl-6-oxo-1,6-dihydropyridin-3-yl)-3H-imidazo[4,5-b]pyridin-3-yl)benzyl)piperazine-1-carbonyl)-2-hydroxybenzaldehyde

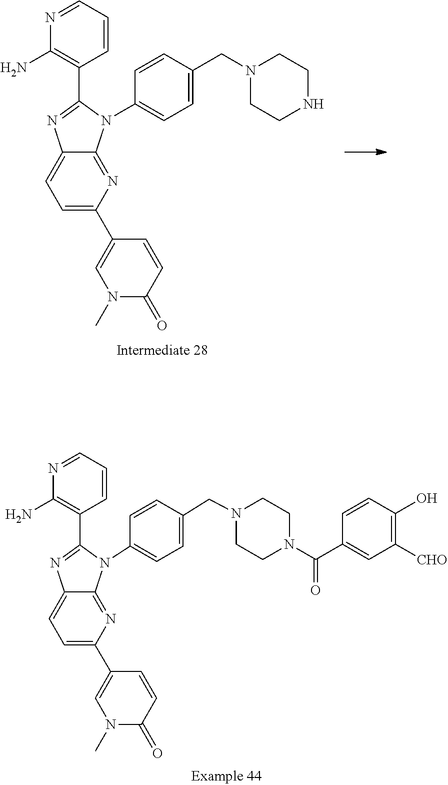 [SMILES: Cn1cc(-c2ccc3nc(-c4cccnc4N)n(-c4ccc(CN5CCNCC5)cc4)c3n2)ccc1=O ; Cn1cc(-c2ccc3nc(-c4cccnc4N)n(-c4ccc(CN5CCN(C(=O)c6ccc(O)c(C=O)c6)CC5)cc4)c3n2)ccc1=O]

To a solution of Intermediate 28 (90.0 mg, 183 □mol) in CH₂Cl₂ (3 mL) were added DIEA (70.8 mg, 548 □mol), HATU (76.4 mg, 201 □mol) and 3-formyl-4-hydroxybenzoic acid (36.4 mg, 219 μmol) at 25 °C The mixture was stirred at 25 °C for 5 hr. The mixture was quenched with H₂O (10 mL) at 0 °C and extracted with CH₂Cl₂ (10 mL×3). The combined organic phases were washed with brine (10 mL), dried over anhydrous Na₂SO₄, filtered and concentrated. After purified by prep-HPLC (column: Phenomenex C18 75×30 mm×3 □m; mobile phase: [water (NH₃H₂O+NH₄HCO₃)-ACN]; gradient: 60%-90% B over 8 min), 5-(4-(4-(2-(2-aminopyridin-3-yl)-5-(1-methyl-6-oxo-1,6-dihydropyridin-3-yl)-3H-imidazo[4,5-b]pyridin-3-yl)benzyl)piperazine-1-carbonyl)-2-hydroxybenzaldehyde (Example 44, 4.2 mg, yield: 3.6%) was obtained as an off white solid. MS: m/z=641.2 [M+H]⁺. ¹H NMR (400 MHz, Dimethylsulfoxide-d₆) δ 10.28 (s, 1H), 8.48 (d, J=2.0 Hz, 1H), 8.23 (d, J=8.4 Hz, 1H), 8.08 (dd, J=9.6, 2.4 Hz, 1H), 8.01-7.94 (m, 1H), 7.84 (d, J=8.4 Hz, 1H), 7.74-7.66 (m, 1H), 7.60-7.54 (m, 1H), 7.51-7.40 (m, 4H), 7.14-6.99 (m, 4H), 6.48 (d, J=9.6 Hz, 1H), 6.37 (dd, J=7.6, 4.4 Hz, 1H), 3.81-3.59 (br s, 2H), 3.58-3.45 (m, 7H), 2.50-2.38 (m, 4H).

### Example 45: 5-(4-(4-(2-(2-Aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde

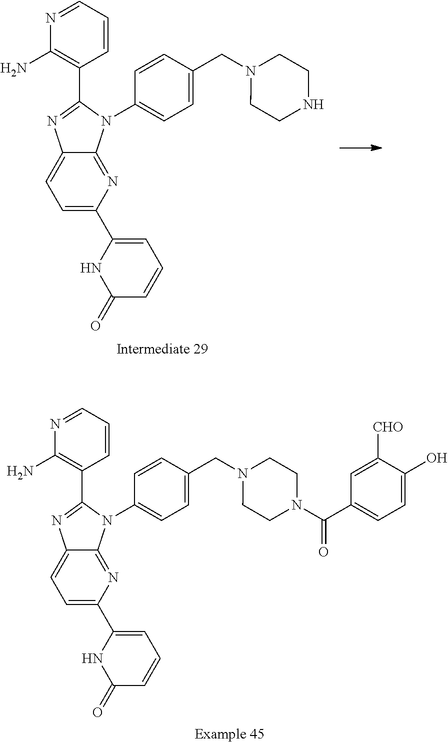 [SMILES: Nc1ncccc1-c1nc2ccc(-c3cccc(=O)[nH]3)nc2n1-c1ccc(CN2CCNCC2)cc1 ; Nc1ncccc1-c1nc2ccc(-c3cccc(=O)[nH]3)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 29. After purified by prep-HPLC (column: Phenomenex C18 150*25 mm*10 μm; mobile phase: [water (NH₄HCO₃)-ACN]; B %: 16%-46%, over 10 min), 5-(4-(4-(2-(2-aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde (Example 45, 8.1 mg, yield: 10%) was obtained as a light-yellow lyophilized powder. MS: m/z=627.2 [M+H]+. ¹H NMR (400 MHz, Dimethylsulfoxide-d₆) δ 10.82 (br s, 1H), 10.27 (s, 1H), 8.33 (d, J=8.4 Hz, 1H), 8.22-8.11 (m, 1H), 8.03-7.97 (m, 1H), 7.68 (d, J=1.6 Hz, 1H), 7.62-7.47 (m, 6H), 7.15 (d, J=6.4 Hz, 2H), 7.05-6.94 (m, 3H), 6.55-6.42 (m, 1H), 6.39 (dd, J=7.6, 4.8 Hz, 1H), 3.64 (s, 2H), 3.59-3.45 (m, 4H), 2.47-2.42 (m, 4H).

### Example 46: 4-(4-(4-(2-(2-Aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde

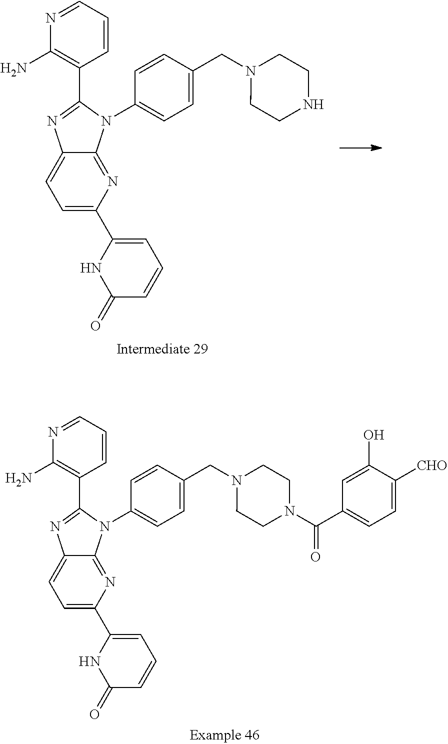 [SMILES: Nc1ncccc1-c1nc2ccc(-c3cccc(=O)[nH]3)nc2n1-c1ccc(CN2CCNCC2)cc1 ; Nc1ncccc1-c1nc2ccc(-c3cccc(=O)[nH]3)nc2n1-c1ccc(CN2CCN(C(=O)c3ccc(C=O)c(O)c3)CC2)cc1]

Following the general procedures described in Example 36, the reaction was carried out using 4-formyl-3-hydroxybenzoic acid and Intermediate 29. After purified by prep-HPLC (column: Phenomenex C18 150*25 mm*10 μm; mobile phase: [water (NH₄HCO₃)-ACN]; B %: 18%-48%, over 10 min), 4-(4-(4-(2-(2-aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde (Example 46, 21.3 mg, yield: 27%) was obtained as a light-yellow lyophilized powder. MS: m/z=627.2 [M+H]+. ¹H NMR (400 MHz, Dimethylsulfoxide-d₆) δ 11.30-10.89 (m, 1H), 10.82 (br s, 1H), 10.29 (s, 1H), 8.34 (d, J=8.4 Hz, 1H), 8.24-8.08 (m, 1H), 8.01 (dd, J=4.8, 1.6 Hz, 1H), 7.69 (d, J=8.0 Hz, 1H), 7.65-7.58 (m, 1H), 7.57-7.31 (m, 5H), 7.16 (dd, J=7.6, 1.6 Hz, 1H), 7.02 (br s, 2H), 6.97-6.93 (m, 1H), 6.90 (d, J=7.6 Hz, 1H), 6.52-6.42 (m, 1H), 6.39 (dd, J=7.6, 4.8 Hz, 1H), 3.64 (s, 4H), 3.38-3.34 (m, 2H), 2.48-2.33 (m, 4H).

### Example 47: 4-((1-(4-(2-(2-Aminopyridin-3-yl)-5-phenyl-3H-imidazo[4,5-b]pyridin-3-yl)benzyl)piperidin-4-yl)amino)-2-hydroxybenzaldehyde

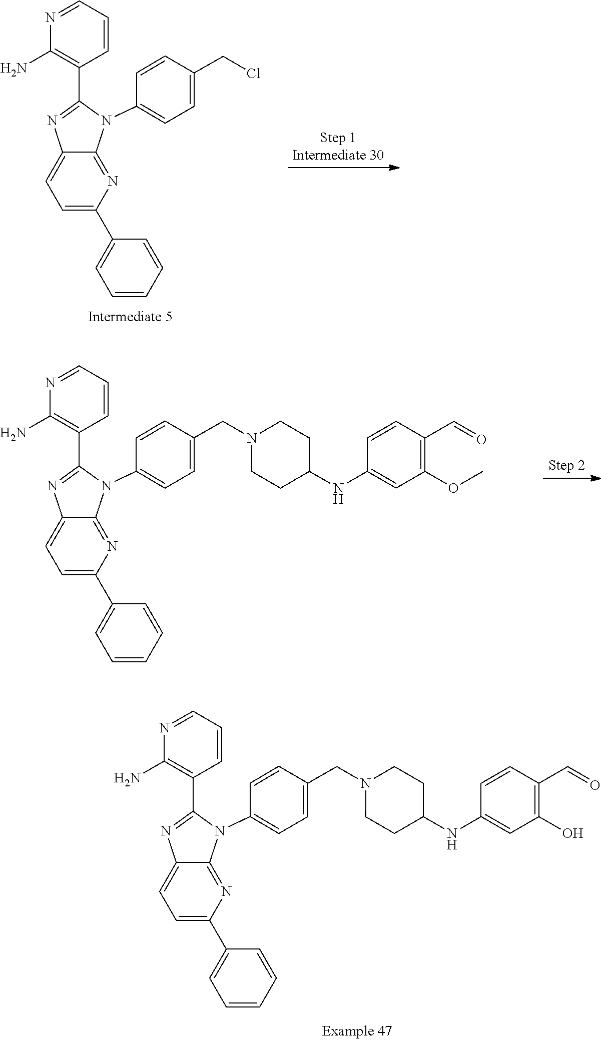 [SMILES: Nc1ncccc1-c1nc2ccc(-c3ccccc3)nc2n1-c1ccc(CCl)cc1 ; COc1cc(NC2CCN(Cc3ccc(-n4c(-c5cccnc5N)nc5ccc(-c6ccccc6)nc54)cc3)CC2)ccc1C=O ; Nc1ncccc1-c1nc2ccc(-c3ccccc3)nc2n1-c1ccc(CN2CCC(Nc3ccc(C=O)c(O)c3)CC2)cc1]

### Step 1: 4-((1-(4-(2-(2-Aminopyridin-3-yl)-5-phenyl-3H-imidazo[4,5-b]pyridin-3-yl)benzyl)piperidin-4-yl)amino)-2-methoxybenzaldehyde

To a solution of Intermediate 5 (105 mg, 256 μmol) and Intermediate 30 (60 mg, 256 μmol, HCl salt) in DMF (1 mL) were added K₂CO₃ (106 mg, 768 μmol) and NaI (3.84 mg, 25.6 μmol). The mixture was stirred at 25 °C for 16 hr. The reaction mixture was diluted with H₂O (20 mL) and extracted with EtOAc (20 mL×2). The combined organic layers were washed with brine (20 mL×2), dried over Na₂SO₄, filtered and concentrated under reduced pressure. After purified by silica gel flash chromatography (Eluent of 50%~100% EtOAc in petroleum ether), 4-((1-(4-(2-(2-aminopyridin-3-yl)-5-phenyl-3H-imidazo[4,5-b]pyridin-3-yl)benzyl)piperidin-4-yl)amino)-2-methoxybenzaldehyde (50 mg, yield: 24% for two steps) was obtained as a yellow solid. MS: m/z=610.2 [M+H]⁺.

### Step 2: 4-((1-(4-(2-(2-Aminopyridin-3-yl)-5-phenyl-3H-imidazo[4,5-b]pyridin-3-yl)benzyl)piperidin-4-yl)amino)-2-hydroxybenzaldehyde

To a solution of 4-((I-(4-(2-(2-aminopyridin-3-yl)-5-phenyl-3H-imidazo[4,5-b]pyridin-3-yl)benzyl)piperidin-4-yl)amino)-2-methoxybenzaldehyde (50 mg, 82 μmol) in CH₂Cl₂ (10 mL) was added BBr₃ (446 mg, 7.42 mmol) at 0 °C The mixture was degassed and purged with N₂ three times and stirred at 25 °C for 16 hr under N₂ atmosphere. The reaction mixture was alkalized with aqueous NaHCO₃ to pH to around 9 and extracted with CH₂Cl₂ (20 mL×2). The combined organic layers were washed with brine (20 mL×2), dried over Na₂SO₄, filtered, and concentrated under reduced pressure. After purified by prep-HPLC (column: Welch Xtimate C₁₈ 150×25 mm×5 μm; mobile phase: [water (HCl)-ACN]; gradient: 15%-45% B over 10 min), 4-((1-(4-(2-(2-aminopyridin-3-yl)-5-phenyl-3H-imidazo[4,5-b]pyridin-3-yl)benzyl)piperidin-4-yl)amino)-2-hydroxybenzaldehyde (Example 47, 6.8 mg, HCl salt, yield: 13%) was obtained as a yellow solid. MS: m/z=596.4 [M+H]⁺. ¹H NMR (400 MHz, Dimethylsulfoxide-d₆) δ 11.04-10.80 (m, 1H), 9.58 (s, 1H), 8.38 (d, J=8.4 Hz, 1H), 8.15-8.05 (m, 5H), 7.88-7.81 (m, 3H), 7.72-7.68 (m, 2H), 7.53-7.36 (m, 5H), 7.24-7.12 (m, 1H), 6.91-6.80 (m, 1H), 6.41-6.25 (m, 1H), 6.13-6.04 (m, 1H), 4.40-4.37 (m, 2H), 3.69-3.59 (m, 1H), 3.49-3.39 (m, 2H), 3.17-3.03 (m, 2H), 2.16-2.11 (m, 2H), 1.92-1.80 (m, 2H).

### Example 48: 5-((1-(4-(2-(2-Aminopyridin-3-yl)-5-phenyl-3H-imidazo[4,5-b]pyridin-3-yl)benzyl)piperidin-4-yl)amino)-2-hydroxybenzaldehyde

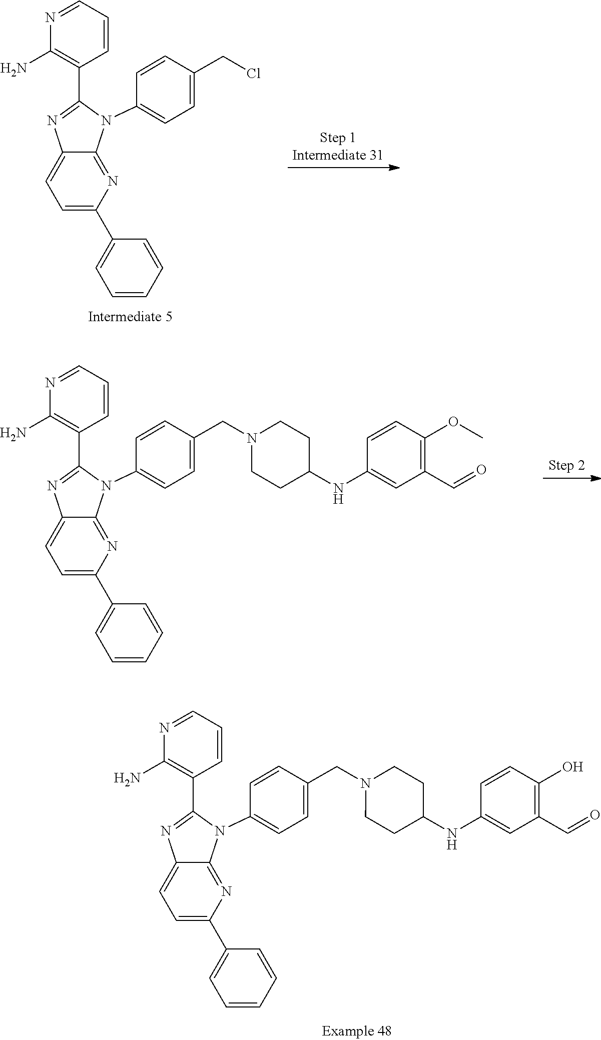 [SMILES: Nc1ncccc1-c1nc2ccc(-c3ccccc3)nc2n1-c1ccc(CCl)cc1 ; COc1ccc(NC2CCN(Cc3ccc(-n4c(-c5cccnc5N)nc5ccc(-c6ccccc6)nc54)cc3)CC2)cc1C=O ; Nc1ncccc1-c1nc2ccc(-c3ccccc3)nc2n1-c1ccc(CN2CCC(Nc3ccc(O)c(C=O)c3)CC2)cc1]

Following the general procedures described in Example 47, the reaction was carried out using Intermediate 5 and Intermediate 31. After purified by prep-HPLC (column: Welch Xtimate C₁₈ 150×25 mm×5 μm; mobile phase: [water (HCl)-ACN]; gradient: 12%-42% B over 10 min), 5-((1-(4-(2-(2-aminopyridin-3-yl)-5-phenyl-3H-imidazo[4,5-b]pyridin-3-yl)benzyl)piperidin-4-yl)amino)-2-hydroxybenzaldehyde (Example 48, 12.1 mg, HCl salt, yield: 6%) was obtained as a brown solid. MS: m/z=596.4 [M+H]⁺. ¹H NMR (400 MHz, Dimethylsulfoxide-d₆) δ 11.02 (br s, 1H), 10.28 (s, 1H), 8.58-8.41 (m, 1H), 8.38 (d, J=8.8 Hz, 1H), 8.15-8.02 (m, 4H), 7.91-7.79 (m, 3H), 7.72-7.65 (m, 2H), 7.56-7.36 (m, 5H), 7.17-7.03 (m, 1H), 6.94-6.86 (m, 1H), 4.36 (s, 2H), 3.66-3.58 (m, 1H), 3.50-3.39 (m, 2H), 3.10-2.91 (m, 2H), 2.24-2.03 (m, 4H).

### Example 49: N-(1-(4-(2-(2-aminopyridin-3-yl)-5-phenyl-3H-imidazo[4,5-b]pyridin-3-yl)benzyl)piperidin-4-yl)-3-formyl-2-hydroxybenzamide

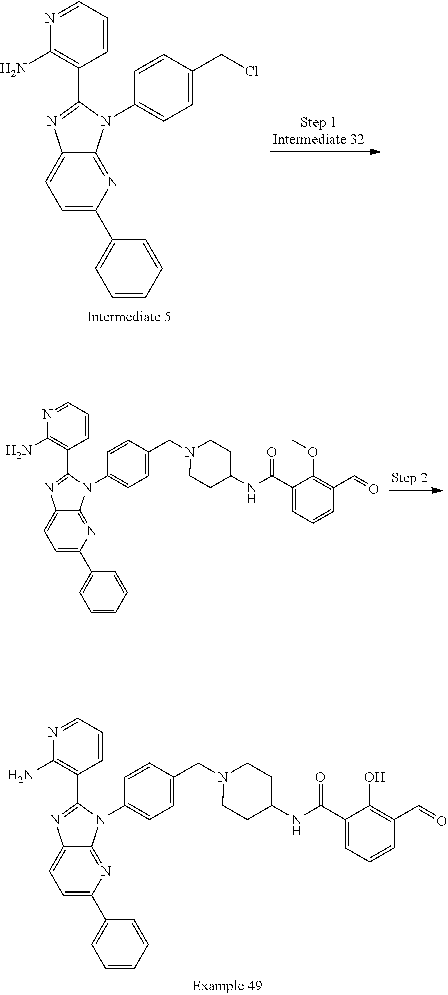 [SMILES: Nc1ncccc1-c1nc2ccc(-c3ccccc3)nc2n1-c1ccc(CCl)cc1 ; COc1c(C=O)cccc1C(=O)NC1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCC(NC(=O)c3cccc(C=O)c3O)CC2)cc1]

Following the general procedures described in Example 47, the reaction was carried out using Intermediate 5 and Intermediate 32. After purified by prep-HPLC (column: Waters xbridge 150×25 mm×10 μm; mobile phase: [water (NH₄HCO₃)-ACN]; gradient: 24%-54% B over 53 min), N-(1-(4-(2-(2-aminopyridin-3-yl)-5-phenyl-3H-imidazo[4,5-b]pyridin-3-yl)benzyl)piperidin-4-yl)-3-formyl-2-hydroxybenzamide (Example 49, 29.7 mg, yield: 15%) was obtained as a light-yellow lyophilized powder. MS: m/z=624.2 [M+H]+. ¹H NMR (400 MHz, Dimethylsulfoxide-d₆) δ 10.33 (s, 1H), 8.27 (d, J=8.4 Hz, 1H), 8.06-7.96 (m, 5H), 7.65-7.55 (m, 2H), 7.54-7.31 (m, 9H), 7.17 (dd, J=7.6, 1.6 Hz, 1H), 7.03 (s, 2H), 6.39 (dd, J=7.6, 4.8 Hz, 1H), 3.90-3.83 (m, 1H), 3.66-3.57 (m, 2H), 2.94-2.83 (m, 2H), 2.47-2.43 (m, 2H), 2.21-2.13 (m, 1H), 1.90-1.82 (m, 2H), 1.67-1.62 (m, 1H).

### Example 50: 3-((I-(4-(2-(2-Aminopyridin-3-yl)-5-phenyl-3H-imidazo[4,5-b]pyridin-3-yl)benzyl)piperidin-4-yl)amino)-2-hydroxybenzaldehyde

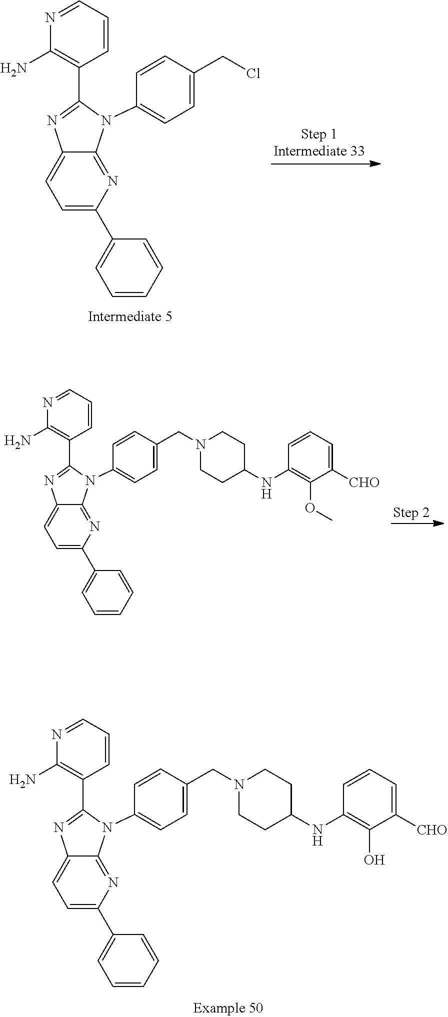 [SMILES: Nc1ncccc1-c1nc2ccc(-c3ccccc3)nc2n1-c1ccc(CCl)cc1 ; COc1c(C=O)cccc1NC1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCC(Nc3cccc(C=O)c3O)CC2)cc1]

Following the general procedures described in Example 47, the reaction was carried out using Intermediate 5 and Intermediate 33. After purified by SFC (column: DAICEL CHIRALCEL OD (250 mm×30 mm, 10 μm); mobile phase: [CO₂-EtOH (0.1% NH₃H₂O)]; B %: 45%, isocratic elution mode), 3-((I-(4-(2-(2-aminopyridin-3-yl)-5-phenyl-3H-imidazo[4,5-b]pyridin-3-yl)benzyl)piperidin-4-yl)amino)-2-hydroxybenzaldehyde (Example 50, 25 mg, yield: 38%) was obtained as a yellow solid. MS: m/z=596.2 [M+H]⁺. ¹H NMR (400 MHz, Dimethylsulfoxide-d₆) δ 9.99 (s, 1H), 8.27 (d, J=8.4 Hz, 1H), 8.06-7.96 (m, 4H), 7.55-7.33 (m, 8H), 7.16 (d, J=7.2 Hz, 1H), 7.03-6.86 (m, 5H), 6.40-6.35 (m, 1H), 3.66-3.52 (m, 2H), 3.27-3.16 (m, 1H), 2.91-2.73 (m, 2H), 2.26-2.09 (m, 2H), 1.98-1.88 (m, 2H), 1.59-1.44 (m, 2H).

### Example 51: 2-((1-(4-(2-(2-Aminopyridin-3-yl)-5-phenyl-3H-imidazo[4,5-b]pyridin-3-yl)benzyl)piperidin-4-yl)amino)-6-hydroxybenzaldehyde

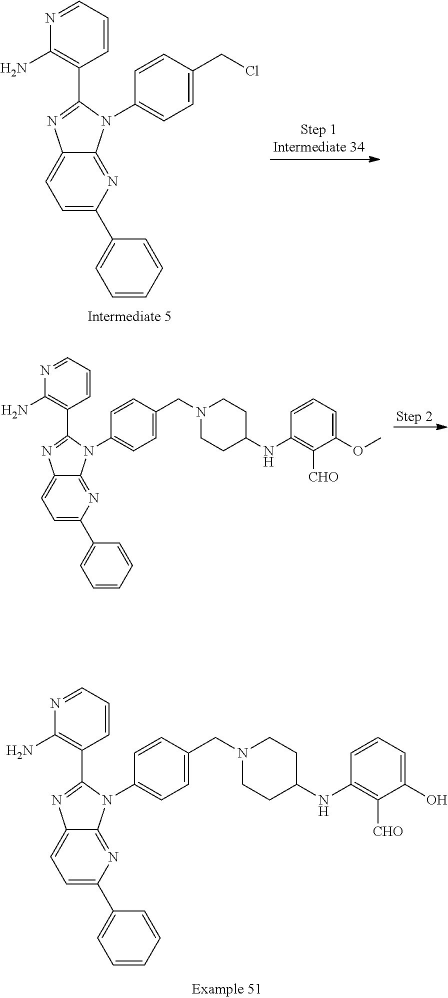 [SMILES: Nc1ncccc1-c1nc2ccc(-c3ccccc3)nc2n1-c1ccc(CCl)cc1 ; COc1cccc(NC2CCN(Cc3ccc(-n4c(-c5cccnc5N)nc5ccc(-c6ccccc6)nc54)cc3)CC2)c1C=O ; Nc1ncccc1-c1nc2ccc(-c3ccccc3)nc2n1-c1ccc(CN2CCC(Nc3cccc(O)c3C=O)CC2)cc1]

Following the general procedures described in Example 47, the reaction was carried out using Intermediate 5 and Intermediate 34. After purified by prep-HPLC (column: Phenomenex C18 80×40 mm×3 μm; mobile phase: [water (NH₃H₂O+NH₄HCO₃)-ACN]; gradient: 55%-85% B over 8 min), 2-((1-(4-(2-(2-aminopyridin-3-yl)-5-phenyl-3H-imidazo[4,5-b]pyridin-3-yl)benzyl)piperidin-4-yl)amino)-6-hydroxybenzaldehyde (Example 51, 12.4 mg, yield: 8.5%) was obtained as a yellow solid. MS: m/z=596.1 [M+H]⁺. ¹H NMR (400 MHz, Dimethylsulfoxide-d₆) δ 10.27-10.26 (m, 2H), 8.80 (d, J=7.2 Hz, 1H), 8.27 (d, J=8.4 Hz, 1H), 8.06-7.96 (m, 4H), 7.51-7.43 (m, 6H), 7.41-7.37 (m, 1H), 7.25-7.14 (m, 2H), 7.02 (br s, 2H), 6.38 (dd, J=7.6, 4.8 Hz, 1H), 6.21 (d, J=8.4 Hz, 1H), 6.04 (d, J=7.6 Hz, 1H), 3.60 (s, 2H), 3.51-3.45 (m, 1H), 2.80-2.69 (m, 2H), 2.33-2.22 (m, 2H), 2.02-1.92 (m, 2H), 1.55-1.41 (m, 2H).

### Example 52: N-(1-(4-(2-(2-Aminopyridin-3-yl)-5-morpholino-3H-imidazo[4,5-b]pyridin-3-yl)benzyl)piperidin-4-yl)-3-formyl-4-hydroxybenzamide

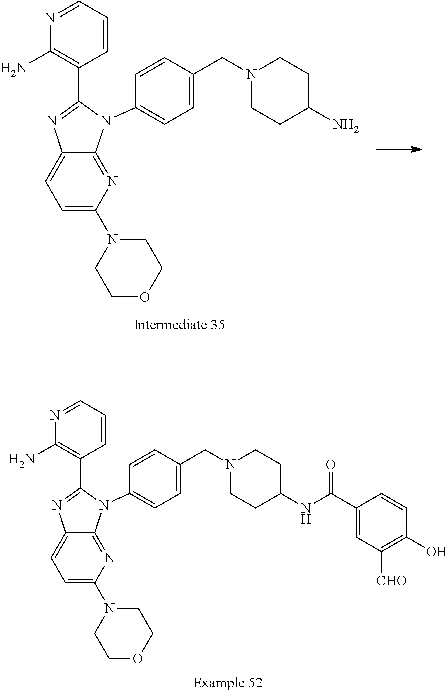 [SMILES: Nc1ncccc1-c1nc2ccc(N3CCOCC3)nc2n1-c1ccc(CN2CCC(N)CC2)cc1 ; Nc1ncccc1-c1nc2ccc(N3CCOCC3)nc2n1-c1ccc(CN2CCC(NC(=O)c3ccc(O)c(C=O)c3)CC2)cc1]

Following the general procedures described in Example 36, the reaction was carried out using 3-formyl-4-hydroxybenzoic acid and Intermediate 35. After purified by prep-HPLC (column: Phenomenex C18 150×25 mm×10 μm; mobile phase: [water (NH₄HCO₃)-ACN]; gradient: 21%-51% B over 10 min), N-(1-(4-(2-(2-aminopyridin-3-yl)-5-morpholino-3H-imidazo[4,5-b]pyridin-3-yl)benzyl)piperidin-4-yl)-3-formyl-4-hydroxybenzamide (Example 52, 6.7 mg, yield: 4.8%) was obtained as a light yellow solid. MS: m/z=633.1 [M+H]⁺. ¹H NMR (400 MHz, Methanol-d₄) δ 10.12 (s, 1H), 8.22 (d, J=2.0 Hz, 1H), 7.99 (dd, J=8.8, 2.4 Hz, 1H), 7.92 (dd, J=6.0, 2.8 Hz, 2H), 7.48 (d, J=8.4 Hz, 2H), 7.35 (d, J=8.0 Hz, 2H), 7.22 (dd, J=7.6, 1.6 Hz, 1H), 7.00 (d, J=8.8 Hz, 1H), 6.88 (d, J=8.8 Hz, 1H), 6.44 (dd, J=7.6, 5.2 Hz, 1H), 3.93-3.86 (m, 1H), 3.78-3.75 (m, 4H), 3.63 (s, 2H), 3.50-3.47 (m, 4H), 3.02-2.95 (m, 2H), 2.26-2.19 (m, 2H), 1.98-1.92 (m, 2H), 1.75-1.67 (m, 2H).

### Example 53: N-(1-(4-(2-(2-Aminopyridin-3-yl)-5-morpholino-3H-imidazo[4,5-b]pyridin-3-yl)benzyl)piperidin-4-yl)-4-formyl-3-hydroxybenzamide

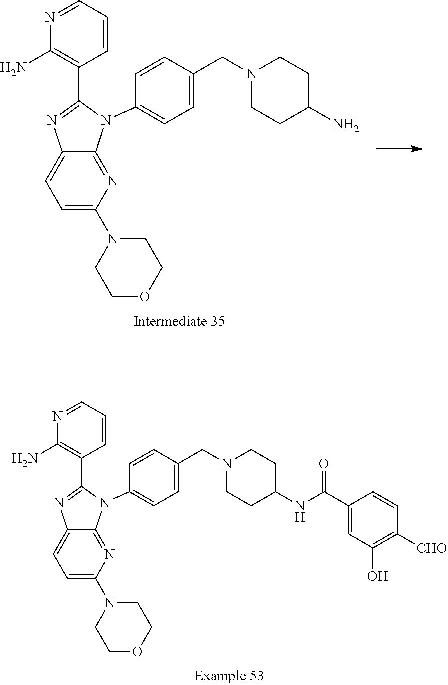 [SMILES: Nc1ncccc1-c1nc2ccc(N3CCOCC3)nc2n1-c1ccc(CN2CCC(N)CC2)cc1 ; Nc1ncccc1-c1nc2ccc(N3CCOCC3)nc2n1-c1ccc(CN2CCC(NC(=O)c3ccc(C=O)c(O)c3)CC2)cc1]

Following the general procedures described in Example 36, the reaction was carried out using 4-formyl-3-hydroxybenzoic acid and Intermediate 35. After purified by silica gel flash chromatography (Eluent of 0%-6% MeOH in CH₂Cl₂) and prep-HPLC (column: Phenomenex C18 150×25 mm×10 μm; mobile phase: [water (NH₄HCO₃)-ACN]; gradient: 24%-54% B over 12 min), N-(1-(4-(2-(2-aminopyridin-3-yl)-5-morpholino-3H-imidazo[4,5-b]pyridin-3-yl)benzyl)piperidin-4-yl)-4-formyl-3-hydroxybenzamide (Example 53, 10.2 mg, yield: 7.5%) was obtained as a light yellow solid. MS: m/z=633.4 [M+H]⁺. ¹H NMR (400 MHz, Methanol-d₄) δ 10.13 (s, 1H), 7.95-7.91 (m, 2H), 7.78 (d, J=8.0 Hz, 1H), 7.48 (d, J=8.4 Hz, 2H), 7.39-7.32 (m, 4H), 7.22 (dd, J=7.6, 1.6 Hz, 1H), 6.89 (d, J=9.2 Hz, 1H), 6.45 (dd, J=7.2, 4.8 Hz, 1H), 3.92-3.87 (m, 1H), 3.78-3.75 (m, 4H), 3.62 (s, 2H), 3.50-3.48 (m, 4H), 3.05-2.92 (m, 2H), 2.25-2.18 (m, 2H), 1.99-1.93 (m, 2H), 1.75-1.66 (m, 2H).

### Example 54: 4-(4-(4-(2-(2-Aminopyridin-3-yl)-6-phenyl-3H-imidazo[4,5-b]pyridin-3-yl)benzyl)piperazin-1-yl)-2-hydroxybenzaldehyde

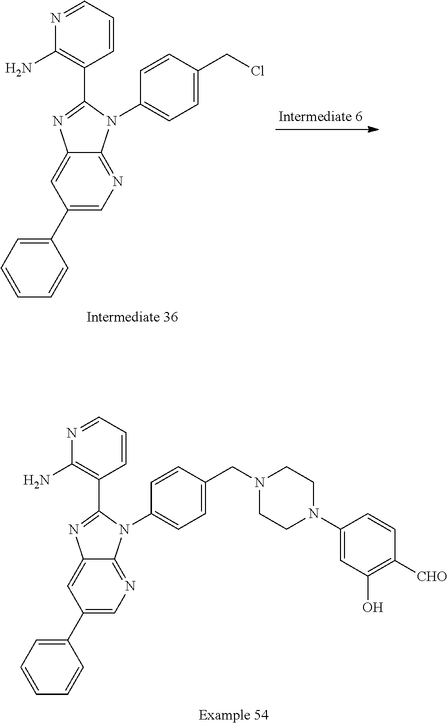 [SMILES: Nc1ncccc1-c1nc2cc(-c3ccccc3)cnc2n1-c1ccc(CCl)cc1 ; Nc1ncccc1-c1nc2cc(-c3ccccc3)cnc2n1-c1ccc(CN2CCN(c3ccc(C=O)c(O)c3)CC2)cc1]

To a solution of Intermediate 36 (200 mg, 486 μmol) and Intermediate 6 (110 mg, 534 μmol) in DMF (2 mL) were added NaI (7.3 mg, 49 μmol) and K₂CO₃ (201 mg, 1.5 mmol).

The mixture was stirred at 80 °C for 2 hr. The reaction mixture was diluted with H₂O (20 mL) and extracted with EtOAc (60 mL). The organic layer was washed with brine (30 mL), dried over Na₂SO₄, filtered, and concentrated under reduced pressure. After purified by prep-HPLC (column: Waters xbridge 150×25 mm 10 μm; mobile phase: [water (NH₄HCO₃)-ACN]; gradient: 52%-82% B over 10 min), 4-(4-(4-(2-(2-aminopyridin-3-yl)-6-phenyl-3H-imidazo[4,5-b]pyridin-3-yl)benzyl)piperazin-1-yl)-2-hydroxybenzaldehyde (Example 54, 15.3 mg, yield: 5%) was obtained as an off white solid. MS: m/z=582.3 [M+H]⁺. ¹H NMR (400 MHz, Dimethylsulfoxide-d₆) δ 11.11 (s, 1H), 9.73 (s, 1H), 8.64 (d, J=1.6 Hz, 1H), 8.47 (d, J=1.6 Hz, 1H), 8.07-7.93 (m, 1H), 7.79 (d, J=7.6 Hz, 2H), 7.55-7.41 (m, 8H), 7.22-7.16 (m, 1H), 7.06 (s, 2H), 6.64-6.58 (m, 1H), 6.38 (dd, J=7.6, 4.8 Hz, 1H), 6.32 (d, J=2.0 Hz, 1H), 3.63 (s, 2H), 3.46-3.40 (m, 4H), 3.34-3.31 (m, 4H).

### Example 55: N-(1-(4-(2-(2-Aminopyridin-3-yl)-5-phenyl-3H-imidazo[4,5-b]pyridin-3-yl)benzyl)piperidin-4-yl)-3-formyl-4-hydroxy-N-methylbenzamide

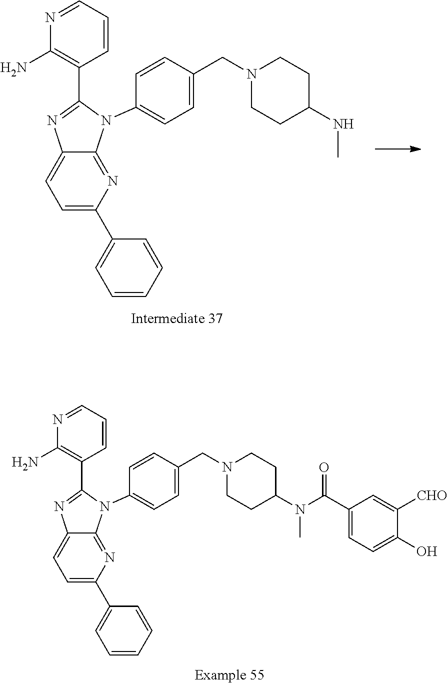 [SMILES: CNC1CCN(Cc2ccc(-n3c(-c4cccnc4N)nc4ccc(-c5ccccc5)nc43)cc2)CC1 ; CN(C(=O)c1ccc(O)c(C=O)c1)C1CCN(Cc2ccc(-n3c(-c4cccnc4N)nc4ccc(-c5ccccc5)nc43)cc2)CC1]

Following the general procedures described in Example 36, the reaction was carried out using 3-formyl-4-hydroxybenzoic acid and Intermediate 37. After purified by prep-HPLC (column: Waters xbridge 150×25 mm 10 μm; mobile phase: [water(NH₄HCO₃)-ACN]; B %: 33%-63%, 14 min), N-(1-(4-(2-(2-aminopyridin-3-yl)-5-phenyl-3H-imidazo[4,5-b]pyridin-3-yl)benzyl)piperidin-4-yl)-3-formyl-4-hydroxy-N-methylbenzamide (Example 55, 6.4 mg, yield: 4%) was obtained as an off white lyophilized powder. MS: m/z=638.3 [M+H]⁺. ¹H NMR (400 MHz, Dimethylsulfoxide-d₆) δ 10.28 (s, 1H), 8.27 (d, J=8.4 Hz, 1H), 8.05-7.96 (m, 4H), 7.72-7.62 (m, 1H), 7.55 (dd, J=8.4, 2.0 Hz, 1H), 7.49-7.37 (m, 7H), 7.17-7.10 (m, 1H), 7.06-6.99 (m, 3H), 6.39-6.31 (m, 1H), 5.75 (s, 1H), 3.56 (br s, 2H), 2.91-2.88 (m, 2H), 2.83 (s, 3H), 2.10-2.05 (m, 2H), 1.94-1.78 (m, 3H), 1.67-1.59 (m, 2H).

### Example 56: 4-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde

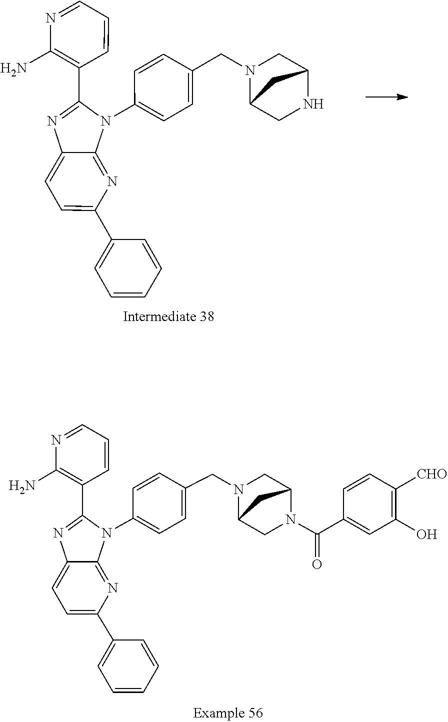 [SMILES: Nc1ncccc1-c1nc2ccc(-c3ccccc3)nc2n1-c1ccc(CN2C[C@H]3C[C@@H]2CN3)cc1 ; Nc1ncccc1-c1nc2ccc(-c3ccccc3)nc2n1-c1ccc(CN2C[C@H]3C[C@@H]2CN3C(=O)c2ccc(C=O)c(O)c2)cc1]

Following the general procedures described in Example 36, the reaction was carried out using 4-formyl-3-hydroxybenzoic acid and Intermediate 38. After purified by prep-HPLC (column: Waters xbridge 150×25 mm×10 μm; mobile phase: [water (NH₃H₂O)-ACN]; gradient: 32%-62% B over 10 min), 4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde (Example 56, 11 mg, yield: 8.4%) was obtained as a light yellow solid. MS: m/z=622.4 [M+H]⁺. ¹H NMR (400 MHz, Chloroform-d) δ 11.11-10.95 (m, 1H), 9.95 (s, 1H), 8.20-8.00 (m, 4H), 7.85-7.77 (m, 1H), 7.68-7.62 (m, 1H), 7.55-7.36 (m, 8H), 7.14-7.08 (m, 2H), 6.60 (br s, 2H), 6.42-6.35 (m, 1H), 3.94-3.84 (m, 2H), 3.58-3.54 (m, 1H), 3.50 (s, 2H), 3.05-2.95 (m, 2H), 2.78-2.72 (m, 1H), 2.02-1.98 (m, 1H), 1.87-1.80 (m, 1H).

### Example 57: 4-((1-(4-(2-(2-Aminopyridin-3-yl)-5-phenyl-3H-imidazo[4,5-b]pyridin-3-yl)benzyl)piperidin-4-yl)(methyl)amino)-2-hydroxybenzaldehyde

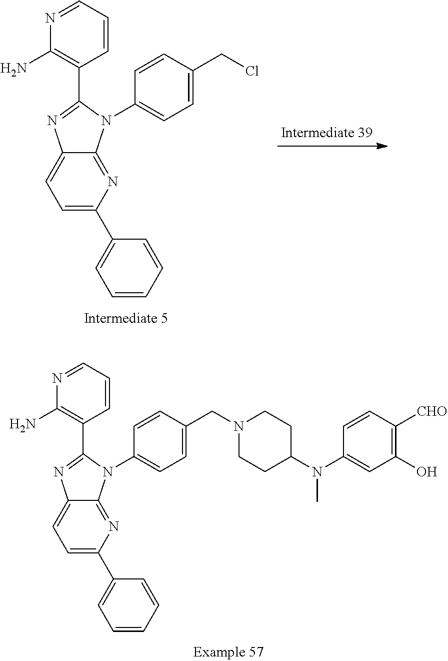 [SMILES: Nc1ncccc1-c1nc2ccc(-c3ccccc3)nc2n1-c1ccc(CCl)cc1 ; CN(c1ccc(C=O)c(O)c1)C1CCN(Cc2ccc(-n3c(-c4cccnc4N)nc4ccc(-c5ccccc5)nc43)cc2)CC1]

Following the general procedures described in Example 54, the reaction was carried out using Intermediate 5 and Intermediate 39. After purified by prep-HPLC (column: Waters xbridge 150×25 mm 10 μm; mobile phase: [water (NH₄HCO₃)-ACN]; gradient: 44%-74% B over 14 min, 4-((1-(4-(2-(2-aminopyridin-3-yl)-5-phenyl-3H-imidazo[4,5-b]pyridin-3-yl)benzyl)piperidin-4-yl)(methyl)amino)-2-hydroxybenzaldehyde (Example 57, 13.1 mg, yield: 6% for two steps) was obtained as a yellow lyophilized powder. MS: m/z=610.4 [M+H]⁺. ¹H NMR (400 MHz, Dimethylsulfoxide-d₆) δ 11.19 (br s, 1H), 9.66 (s, 1H), 8.27 (d, J=8.4 Hz, 1H), 8.07-7.95 (m, 4H), 7.52-7.36 (m, 8H), 7.16 (dd, J=7.6, 1.6 Hz, 1H), 7.01 (s, 2H), 6.49 (dd, J=8.8, 2.0 Hz, 1H), 6.39 (dd, J=7.6, 4.8 Hz, 1H), 6.17 (d, J=2.0 Hz, 1H), 3.84-3.71 (m, 1H), 3.61 (s, 2H), 2.94 (d, J=11.6 Hz, 2H), 2.85 (s, 3H), 2.25-2.17 (m, 2H), 1.89-1.77 (m, 2H), 1.63-1.62 (m, 2H).

### Example 58: (R)—N-(1-(4-(2-(2-Aminopyridin-3-yl)-5-phenyl-3H-imidazo[4,5-b]pyridin-3-yl)benzyl)piperidin-3-yl)-3-formyl-4-hydroxybenzamide

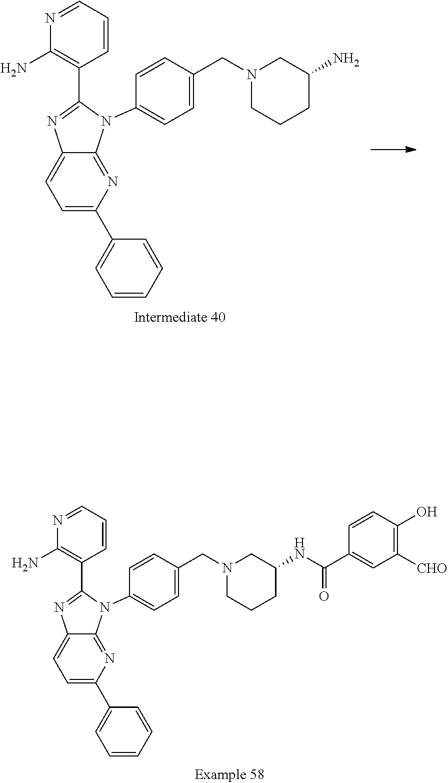 [SMILES: Nc1ncccc1-c1nc2ccc(-c3ccccc3)nc2n1-c1ccc(CN2CCC[C@@H](N)C2)cc1 ; Nc1ncccc1-c1nc2ccc(-c3ccccc3)nc2n1-c1ccc(CN2CCC[C@@H](NC(=O)c3ccc(O)c(C=O)c3)C2)cc1]

Following the general procedures described in Example 36, the reaction was carried out using 3-formyl-4-hydroxybenzoic acid and Intermediate 40. After purified by prep-HPLC (column: Waters xbridge 150×25 mm 10 μm; mobile phase: [water(NH₄HCO₃)-ACN]; gradient: 40%-70% B over 14 min), (R)—N-(1-(4-(2-(2-aminopyridin-3-yl)-5-phenyl-3H-imidazo[4,5-b]pyridin-3-yl)benzyl)piperidin-3-yl)-3-formyl-4-hydroxybenzamide (Example 58, 6.1 mg, yield: 9%) was obtained as a yellow solid. MS: m/z=624.5 [M+H]⁺. ¹H NMR (400 MHz, Dimethylsulfoxide-d₆) δ 10.29 (s, 1H), 8.26 (d, J=8.4 Hz, 1H), 8.21-8.14 (m, 2H), 8.04-7.96 (m, 4H), 7.92-7.90 (m, 1H), 7.53-7.48 (m, 2H), 7.47-7.39 (m, 5H), 7.39-7.34 (m, 1H), 7.11 (dd, J=7.6, 2.0 Hz, 1H), 7.07 (br s, 2H), 7.00-6.94 (m, 1H), 6.36 (dd, J=8.0, 4.8 Hz, 1H), 4.04-3.88 (m, 1H), 3.69-3.62 (m, 1H), 3.58-3.51 (m, 1H), 2.90-2.75 (m, 2H), 2.01-1.92 (m, 2H), 1.86-1.75 (m, 2H), 1.64-1.49 (m, 2H).

### Example 59: (R)—N-(1-(4-(2-(2-aminopyridin-3-yl)-5-phenyl-3H-imidazo[4,5-b]pyridin-3-yl)benzyl)pyrrolidin-3-yl)-3-formyl-4-hydroxybenzamide

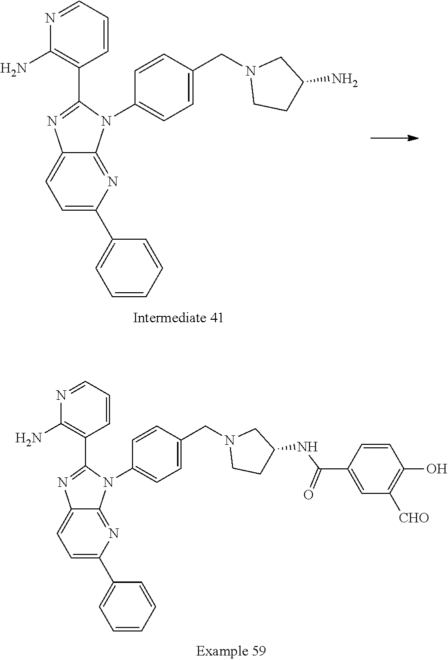 [SMILES: Nc1ncccc1-c1nc2ccc(-c3ccccc3)nc2n1-c1ccc(CN2CC[C@@H](N)C2)cc1 ; Nc1ncccc1-c1nc2ccc(-c3ccccc3)nc2n1-c1ccc(CN2CC[C@@H](NC(=O)c3ccc(O)c(C=O)c3)C2)cc1]

Following the general procedures described in Example 36, the reaction was carried out using 3-formyl-4-hydroxybenzoic acid and Intermediate 41. After purified by prep-HPLC (column: Waters xbridge 150×25 mm 10 μm; mobile phase: [water(NH₄HCO₃)-ACN]; gradient: 35%-65% B over 14 min), (R)—N-(1-(4-(2-(2-aminopyridin-3-yl)-5-phenyl-3H-imidazo[4,5-b]pyridin-3-yl)benzyl)pyrrolidin-3-yl)-3-formyl-4-hydroxybenzamide (Example 59, 6.1 mg, yield: 9%) was obtained as a yellow solid. MS: m/z=610.4 [M+H]⁺. ¹H NMR (400 MHz, Dimethylsulfoxide-d₆) δ 10.30 (s, 1H), 8.53-8.47 (m, 1H), 8.27 (d, J=8.4 Hz, 1H), 8.23 (d, J=2.4 Hz, 1H), 8.05-7.97 (m, 4H), 7.96-7.94 (m, 1H), 7.54-7.50 (m, 2H), 7.48-7.41 (m, 5H), 7.41-7.37 (m, 1H), 7.16-7.13 (m, 1H), 7.04 (br s, 2H), 7.02-6.98 (m, 1H), 6.36 (dd, J=8.0, 4.8 Hz, 1H), 4.47-4.35 (m, 1H), 3.70 (d, J=6.8 Hz, 2H), 2.86-2.79 (m, 2H), 2.24-2.10 (m, 2H), 2.02-1.69 (m, 2H).

### Example 60: N-(1-(4-(2-(2-Aminopyridin-3-yl)-5-phenyl-3H-imidazo[4,5-b]pyridin-3-yl)benzyl)piperidin-4-yl)-4-formyl-3-hydroxy-N-methylbenzamide

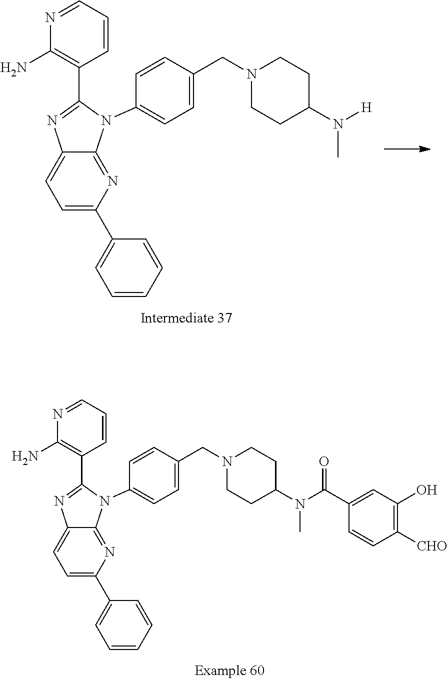 [SMILES: CNC1CCN(Cc2ccc(-n3c(-c4cccnc4N)nc4ccc(-c5ccccc5)nc43)cc2)CC1 ; CN(C(=O)c1ccc(C=O)c(O)c1)C1CCN(Cc2ccc(-n3c(-c4cccnc4N)nc4ccc(-c5ccccc5)nc43)cc2)CC1]

Following the general procedures described in Example 36, the reaction was carried out using 4-formyl-3-hydroxybenzoic acid and Intermediate 37. After purified by prep-HPLC (column: Waters xbridge 150×25 mm×10 μm; mobile phase: [water (NH₄HCO₃)-ACN]; gradient: 39%-69% B over 10 min), N-(1-(4-(2-(2-aminopyridin-3-yl)-5-phenyl-3H-imidazo[4,5-b]pyridin-3-yl)benzyl)piperidin-4-yl)-4-formyl-3-hydroxy-N-methylbenzamide (Example 60, 38.7 mg, yield: 33%) was obtained as a light yellow solid. MS: m/z=638.4 [M+H]⁺. ¹H NMR (400 MHz, Dimethylsulfoxide-d₆) δ 10.28 (s, 1H), 8.28 (d, J=8.0 Hz, 1H), 8.05-7.95 (m, 4H), 7.79 (d, J=7.6 Hz, 1H), 7.48-7.35 (m, 7H), 7.15-7.85 (m, 5H), 6.40-6.26 (m, 1H), 3.60-3.50 (m, 2H), 3.05-2.75 (m, 4H), 2.15-2.10 (m, 1H), 1.90-1.55 (m, 6H).

### Example 61: N-(1-(4-(2-(2-Aminopyridin-3-yl)-5-phenyl-3H-imidazo[4,5-b]pyridin-3-yl)benzyl)azetidin-3-yl)-3-formyl-4-hydroxybenzamide

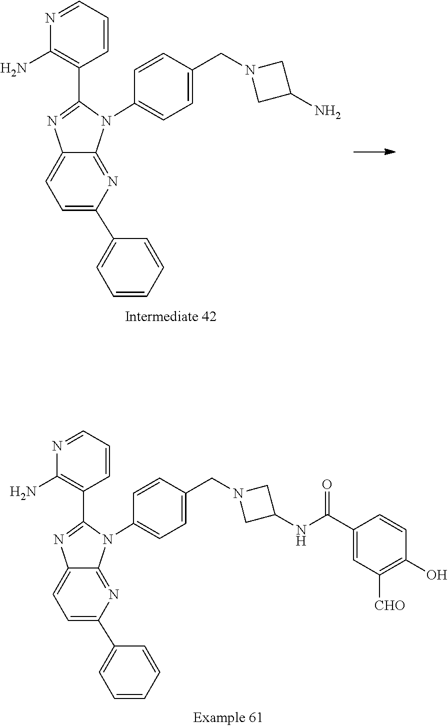 [SMILES: Nc1ncccc1-c1nc2ccc(-c3ccccc3)nc2n1-c1ccc(CN2CC(N)C2)cc1 ; Nc1ncccc1-c1nc2ccc(-c3ccccc3)nc2n1-c1ccc(CN2CC(NC(=O)c3ccc(O)c(C=O)c3)C2)cc1]

Following the general procedures described in Example 36, the reaction was carried out using 3-formyl-4-hydroxybenzoic acid and Intermediate 42. After purified by prep-HPLC (column: Waters xbridge 150×25 mm 10 μm; mobile phase: [water (NH₄HCO₃)-ACN]; gradient: 27%-57% B over 14 min), N-(1-(4-(2-(2-aminopyridin-3-yl)-5-phenyl-3H-imidazo[4,5-b]pyridin-3-yl)benzyl)azetidin-3-yl)-3-formyl-4-hydroxybenzamide (Example 61, 7.7 mg, yield: 12%) was obtained as a yellow solid. MS: m/z=596.4 [M+H]⁺. ¹H NMR (400 MHz, Dimethylsulfoxide-d₆) δ 10.30 (s, 1H), 8.83-8.76 (m, 1H), 8.27 (d, J=8.4 Hz, 1H), 8.24-8.20 (m, 1H), 8.07-7.95 (m, 5H), 7.51-7.36 (m, 8H), 7.16 (dd, J=7.6, 2.0 Hz, 1H), 7.03-6.97 (m, 3H), 6.39 (dd, J=7.6, 4.8 Hz, 1H), 4.57-4.44 (m, 1H), 3.71 (s, 2H), 3.63-3.57 (m, 2H), 3.16-3.09 (m, 2H).

### Example 62: 5-(6-(4-(2-(2-Aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde

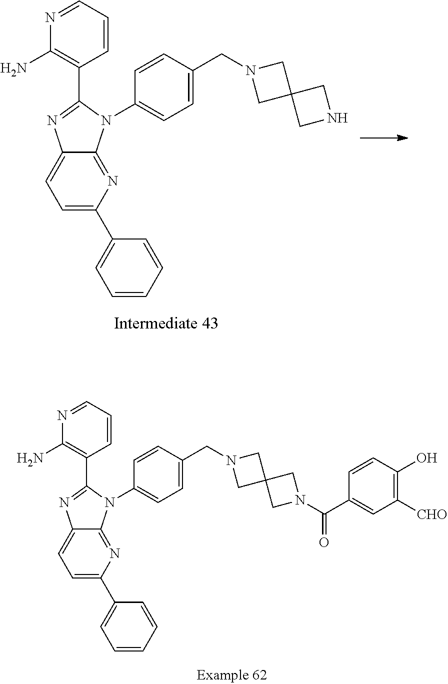 [SMILES: Nc1ncccc1-c1nc2ccc(-c3ccccc3)nc2n1-c1ccc(CN2CC3(CNC3)C2)cc1 ; Nc1ncccc1-c1nc2ccc(-c3ccccc3)nc2n1-c1ccc(CN2CC3(C2)CN(C(=O)c2ccc(O)c(C=O)c2)C3)cc1]

Following the general procedures described in Example 36, the reaction was carried out using 3-formyl-4-hydroxybenzoic acid and Intermediate 43. After purified by silica gel flash chromatography (Eluent of 0%-8% MeOH in CH₂Cl₂), 5-(6-(4-(2-(2-Aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde (Example 62, 9.1 mg, yield: 12%) was obtained as a yellow solid. MS: m/z=622.2 [M+H]⁺. ¹H NMR (400 MHz, Dimethylsulfoxide-d₆) δ 11.44-11.06 (m, 1H), 10.27 (s, 1H), 8.27 (d, J=8.4 Hz, 1H), 8.05-7.96 (m, 4H), 7.94-7.91 (m, 1H), 7.83-7.71 (m, 1H), 7.49-7.38 (m, 7H), 7.15 (d, J=7.6 Hz, 1H), 7.00 (br s, 3H), 6.40 (dd, J=8.0, 4.8 Hz, 1H), 4.55-4.30 (m, 2H), 4.22-4.04 (m, 2H), 3.62 (s, 2H), 3.37-3.34 (m, 4H).

### Example 63: (R)—N-(1-(4-(2-(2-Aminopyridin-3-yl)-5-phenyl-3H-imidazo[4,5-b]pyridin-3-yl)benzyl)piperidin-3-yl)-4-formyl-3-hydroxybenzamide

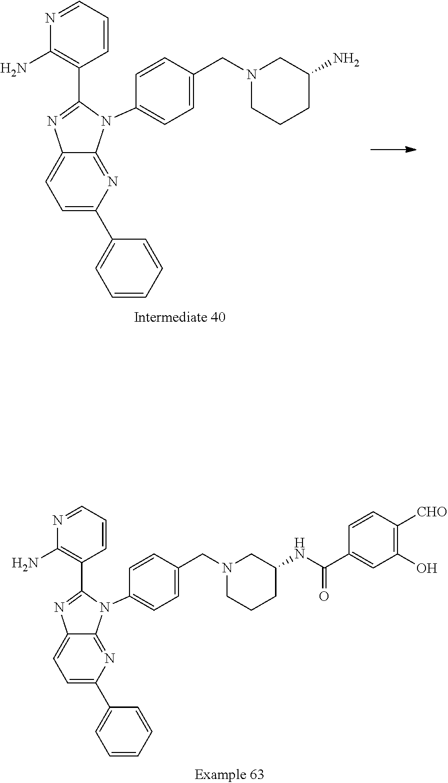 [SMILES: Nc1ncccc1-c1nc2ccc(-c3ccccc3)nc2n1-c1ccc(CN2CCC[C@@H](N)C2)cc1 ; Nc1ncccc1-c1nc2ccc(-c3ccccc3)nc2n1-c1ccc(CN2CCC[C@@H](NC(=O)c3ccc(C=O)c(O)c3)C2)cc1]

Following the general procedures described in Example 36, the reaction was carried out using 4-formyl-3-hydroxybenzoic acid and Intermediate 40. After purified by prep-HPLC (column: Waters xbridge 150×25 mm 10 μm; mobile phase: [water(NH₄HCO₃)-ACN]; gradient: 36%-66% B over 14 min), (R)—N-(1-(4-(2-(2-aminopyridin-3-yl)-5-phenyl-3H-imidazo[4,5-b]pyridin-3-yl)benzyl)piperidin-3-yl)-4-formyl-3-hydroxybenzamide (Example 63, 7.2 mg, yield: 10%) was obtained as a yellow solid. MS: m/z=624.4 [M+H]⁺. ¹H NMR (400 MHz, Dimethylsulfoxide-d₆) δ 10.31 (s, 1H), 8.35 (d, J=7.6 Hz, 1H), 8.26 (d, J=8.0 Hz, 1H), 8.05-7.96 (m, 3H), 7.93 (dd, J=4.8, 1.6 Hz, 1H), 7.70 (d, J=8.0 Hz, 1H), 7.53-7.48 (m, 2H), 7.48-7.39 (m, 6H), 7.39-7.33 (m, 2H), 7.12 (dd, J=7.6, 1.6 Hz, 1H), 7.06 (s, 2H), 6.35 (dd, 1=7.6, 4.8 Hz, 1H), 4.04-3.90 (m, 1H), 3.69-3.62 (m, 1H), 3.60-3.52 (m, 1H), 2.89-2.83 (m, 1H), 2.80-2.75 (m, 1H), 2.02-1.92 (m, 2H), 1.85-1.79 (m, 1H), 1.77-1.70 (m, 1H), 1.61-1.51 (m, 1H), 1.44-1.33 (m, 1H).

### Example 64: (R)—N-(1-(4-(2-(2-Aminopyridin-3-yl)-5-phenyl-3H-imidazo[4,5-b]pyridin-3-yl)benzyl)pyrrolidin-3-yl)-4-formyl-3-hydroxybenzamide

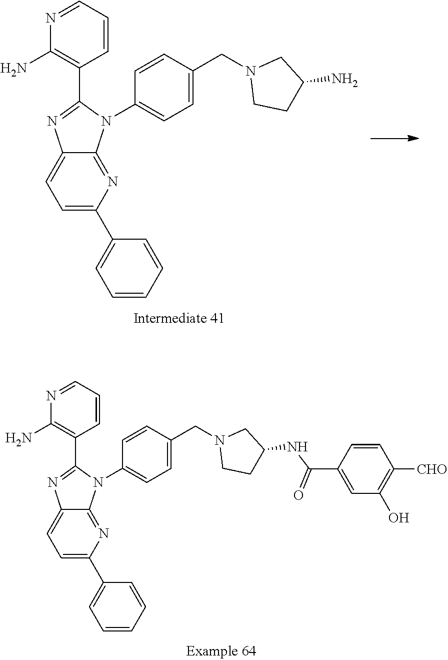 [SMILES: Nc1ncccc1-c1nc2ccc(-c3ccccc3)nc2n1-c1ccc(CN2CC[C@@H](N)C2)cc1 ; Nc1ncccc1-c1nc2ccc(-c3ccccc3)nc2n1-c1ccc(CN2CC[C@@H](NC(=O)c3ccc(C=O)c(O)c3)C2)cc1]

Following the general procedures described in Example 36, the reaction was carried out using 4-formyl-3-hydroxybenzoic acid and Intermediate 41. After purified by prep-HPLC (column: Waters xbridge 150×25 mm 10 μm; mobile phase: [water(NH₄HCO₃)-ACN]; gradient: 36%-66% B over 14 min), (R)—N-(1-(4-(2-(2-aminopyridin-3-yl)-5-phenyl-3H-imidazo[4,5-h]pyridin-3-yl)benzyl)pyrrolidin-3-yl)-4-formyl-3-hydroxybenzamide (Example 64, 16.2 mg, yield: 23%) was obtained as a yellow solid. MS: m/z=610.4 [M+H]⁺. ¹H NMR (400 MHz, Dimethylsulfoxide-d₆) δ 10.97-10.79 (m, 1H), 10.31 (s, 1H), 8.65 (d, J=7.2 Hz, 11H), 8.27 (d, J=8.4 Hz, 1H), 8.05-7.93 (m, 4H), 7.70 (d, J=8.0 Hz, 1H), 7.54-7.49 (m, 2H), 7.47-7.41 (m, 5H), 7.41-7.35 (m, 2H), 7.14 (dd, J=7.6, 1.6 Hz, 1H), 7.03 (s, 2H), 6.35 (dd, J=8.0, 5.2 Hz, 1H), 4.45-4.33 (m, 1H), 3.70 (d, J=5.6 Hz, 2H), 3.30-3.23 (m, 2H), 2.87-2.80 (m, 1H), 2.73-2.68 (m, 1H), 2.24-2.11 (m, 1H), 1.90-1.77 (m, 1H).

### Example 65: N-(1-(4-(2-(2-Aminopyridin-3-yl)-5-phenyl-3H-imidazo[4,5-h]pyridin-3-yl)benzyl)azetidin-3-yl)-4-formyl-3-hydroxybenzamide

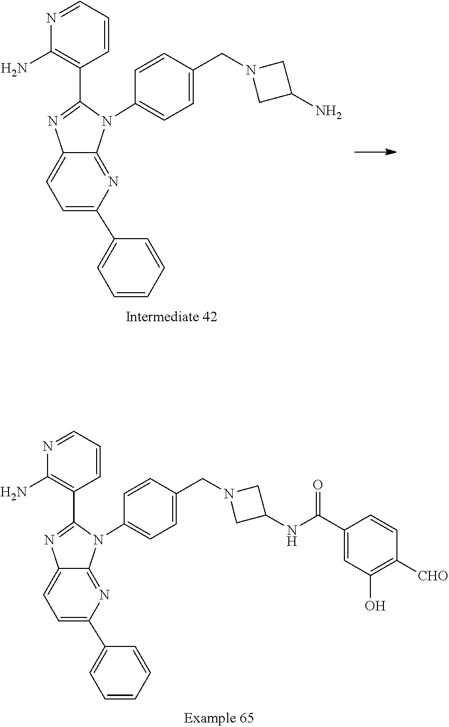 [SMILES: Nc1ncccc1-c1nc2ccc(-c3ccccc3)nc2n1-c1ccc(CN2CC(N)C2)cc1 ; Nc1ncccc1-c1nc2ccc(-c3ccccc3)nc2n1-c1ccc(CN2CC(NC(=O)c3ccc(C=O)c(O)c3)C2)cc1]

Following the general procedures described in Example 36, the reaction was carried out using 4-formyl-3-hydroxybenzoic acid and Intermediate 42. After purified by prep-HPLC (column: Waters xbridge 150×25 mm 10 μm; mobile phase: [water(NH₄HCO₃)-ACN]; gradient: 32%-62% B over 14 min), N-(1-(4-(2-(2-aminopyridin-3-yl)-5-phenyl-3H-imidazo[4,5-h]pyridin-3-yl)benzyl)azetidin-3-yl)-4-formyl-3-hydroxybenzamide (Example 65, 8.1 mg, yield: 11%) was obtained as a yellow solid. MS: m/z=596.4 [M+H]⁺. ¹H NMR (400 MHz, Dimethylsulfoxide-d₆) δ 11.04-10.79 (m, 1H), 10.31 (s, 1H), 8.97 (d, J=7.2 Hz, 1H), 8.27 (d, J=8.4 Hz, 1H), 8.05-8.01 (m, 2H), 8.01-7.96 (m, 2H), 7.72 (d, J=8.0 Hz, 1H), 7.49-7.37 (m, 9H), 7.16 (dd, J=8.0, 2.0 Hz, 1H), 7.00 (s, 2H), 6.39 (dd, J=7.6, 4.8 Hz, 1H), 4.54-4.44 (m, 1H), 3.71 (s, 2H), 3.66-3.57 (m, 2H), 3.17-3.09 (m, 2H).

### Example 66: 4-(6-(4-(2-(2-Aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde

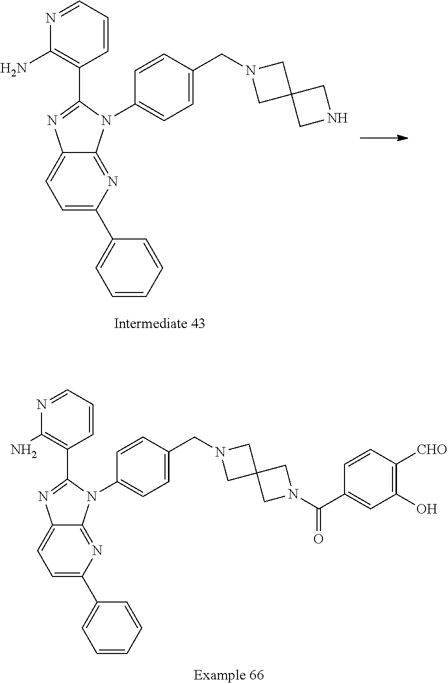 [SMILES: Nc1ncccc1-c1nc2ccc(-c3ccccc3)nc2n1-c1ccc(CN2CC3(CNC3)C2)cc1 ; Nc1ncccc1-c1nc2ccc(-c3ccccc3)nc2n1-c1ccc(CN2CC3(C2)CN(C(=O)c2ccc(C=O)c(O)c2)C3)cc1]

Following the general procedures described in Example 36, the reaction was carried out using 4-formyl-3-hydroxybenzoic acid and Intermediate 43. After purified by silica gel flash chromatography (Eluent of 0%-8% MeOH in CH₂C2), 4-(6-(4-(2-(2-aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde (Example 66, 5.2 mg, yield: 8%) was obtained as a yellow solid. MS: m/z=622.4 [M+H]⁺. ¹H NMR (400 MHz, Dimethylsulfoxide-d₆) δ 10.30 (s, 1H), 8.33-8.22 (m, 1H), 8.04-7.97 (m, 4H), 7.64 (d, J=8.0 Hz, 1H), 7.49-7.38 (m, 8H), 7.18-7.12 (m, 2H), 7.09-7.03 (m, 1H), 7.00 (s, 2H), 6.40 (dd, J=7.6, 4.8 Hz, 1H), 4.36 (s, 2H), 4.13 (s, 2H), 3.61 (s, 2H), 3.38-3.35 (m, 4H).

### Example 67: 5-(7-(4-(2-(2-Aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde

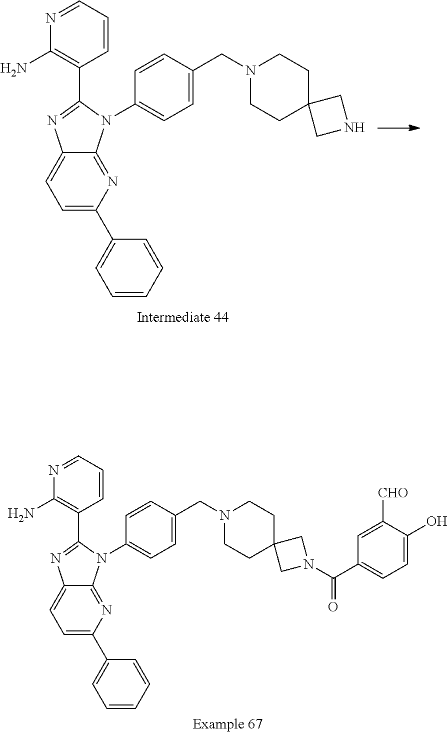 [SMILES: Nc1ncccc1-c1nc2ccc(-c3ccccc3)nc2n1-c1ccc(CN2CCC3(CC2)CNC3)cc1 ; Nc1ncccc1-c1nc2ccc(-c3ccccc3)nc2n1-c1ccc(CN2CCC3(CC2)CN(C(=O)c2ccc(O)c(C=O)c2)C3)cc1]

Following the general procedures described in Example 36, the reaction was carried out using 3-formyl-4-hydroxybenzoic acid and Intermediate 44. After purified by prep-HPLC (column: Phenomenex C18 150×25 mm×10 μm; mobile phase: [water (NH₃H₂O)-ACN]; gradient: 26%-56%, over 12 min), 5-(7-(4-(2-(2-aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde (Example 67, 25.8 mg, yield: 19%) was obtained as a light-yellow solid. MS: m/z=650.5 [M+H]⁺. ¹H NMR (400 MHz, Dimethylsulfoxide-d₆) δ 10.27 (s, 1H), 8.27 (d, J=8.4 Hz, 1H), 8.04-8.01 (m, 2H), 7.99-7.97 (m, 2H), 7.94 (d, J=2.0 Hz, 1H), 7.80 (dd, J=8.8, 2.0 Hz, 1H), 7.48-7.44 (m, 7H), 7.41-7.38 (m, 1H), 7.15 (dd, J=7.6, 1.6 Hz, 1H), 7.03 (br s, 2H), 6.99 (d, J=8.8 Hz, 1H), 6.36 (dd, J=7.6, 4.8 Hz, 1H), 4.06 (s, 2H), 3.77 (s, 2H), 3.55 (s, 2H), 2.35-2.30 (m, 2H), 1.84-1.67 (m, 6H).

### Example 68: N-(7-(4-(2-(2-Aminopyridin-3-yl)-5-phenyl-3H-imidazo[4,5-b]pyridin-3-yl)benzyl)-7-azaspiro[3.5]nonan-2-yl)-3-formyl-4-hydroxybenzamide

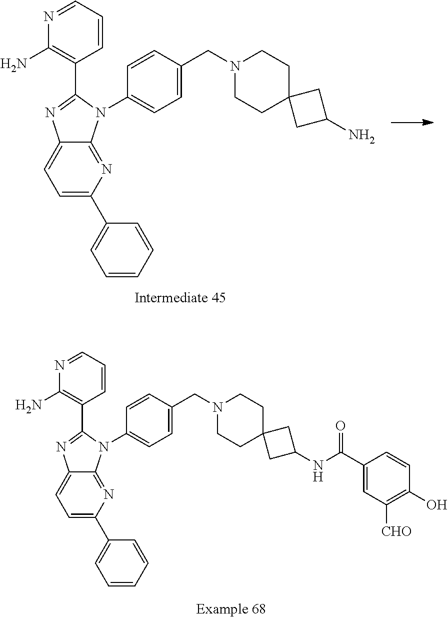 [SMILES: Nc1ncccc1-c1nc2ccc(-c3ccccc3)nc2n1-c1ccc(CN2CCC3(CC2)CC(N)C3)cc1 ; Nc1ncccc1-c1nc2ccc(-c3ccccc3)nc2n1-c1ccc(CN2CCC3(CC2)CC(NC(=O)c2ccc(O)c(C=O)c2)C3)cc1]

Following the general procedures described in Example 36, the reaction was carried out using 3-formyl-4-hydroxybenzoic acid and Intermediate 45. After purified by prep-HPLC (column: Phenomenex C18 150×25 mm×10 μm; mobile phase: [water (NH₄HCO₃)-ACN]; gradient: 35%-55% B over 14 min), N-(7-(4-(2-(2-aminopyridin-3-yl)-5-phenyl-3H-imidazo[4,5-b]pyridin-3-yl)benzyl)-7-azaspiro[3.5]nonan-2-yl)-3-formyl-4-hydroxybenzamide (Example 68, 8.3 mg, yield: 6.1%) was obtained as a light-yellow solid. MS: m/z=664.4 [M+H]⁺. ¹H NMR (400 MHz, Dimethylsulfoxide-d₆)S 10.29 (s, 1H), 8.59-8.51 (m, 1H), 8.27 (d, J=8.0 Hz, 1H), 8.21-8.17 (m, 1H), 8.05-7.97 (m, 6H), 7.47-7.43 (m, 6H), 7.40-7.37 (m, 1H), 7.17-7.14 (m, 1H), 7.03 (s, 2H), 6.98 (d, J=8.4 Hz, 1H), 6.38 (dd, J=7.6, 4.8 Hz, 1H), 4.42-4.29 (m, 1H), 3.54 (s, 2H), 2.17-2.14 (m, 4H), 1.88-1.77 (m, 4H), 1.63-1.57 (m, 4H).

### Example 69: N-(2-(4-(2-(2-Aminopyridin-3-yl)-5-phenyl-3H-imidazo[4,5-b]pyridin-3-yl)benzyl)-2-azaspiro[3.3]heptan-6-yl)-3-formyl-4-hydroxybenzamide

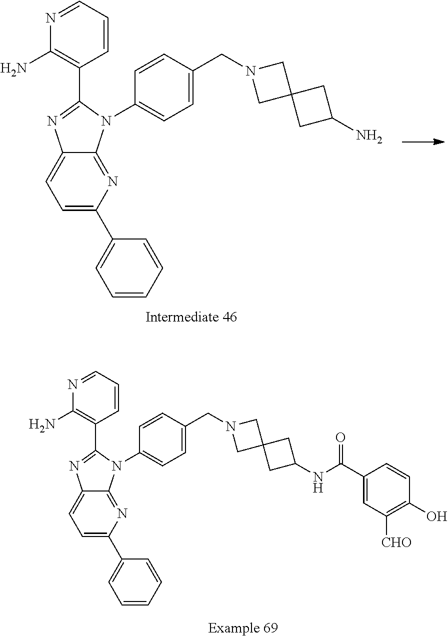 [SMILES: Nc1ncccc1-c1nc2ccc(-c3ccccc3)nc2n1-c1ccc(CN2CC3(CC(N)C3)C2)cc1 ; Nc1ncccc1-c1nc2ccc(-c3ccccc3)nc2n1-c1ccc(CN2CC3(CC(NC(=O)c4ccc(O)c(C=O)c4)C3)C2)cc1]

Following the general procedures described in Example 36, the reaction was carried out using 3-formyl-4-hydroxybenzoic acid and Intermediate 46. After purified by prep-HPLC (column: Phenomenex C18 150×25 mm×10 μm; mobile phase: [water (NH₄HCO₃)-ACN]; gradient: 28%-58% B over 10 min), N-(2-(4-(2-(2-aminopyridin-3-yl)-5-phenyl-3H-imidazo[4,5-b]pyridin-3-yl)benzyl)-2-azaspiro[3.3]heptan-6-yl)-3-formyl-4-hydroxybenzamide (Example 69, 12 mg, yield: 6%) was obtained as an off white lyophilized powder. MS: m/z=636.4 [M+H]⁺. ¹H NMR (400 MHz, Methanol-d₄) δ 10.12 (s, 1H), 8.25-8.14 (m, 2H), 8.04-8.01 (m, 2H), 8.00-7.93 (m, 3H), 7.53-7.46 (m, 4H), 7.43-7.32 (m, 4H), 7.01-6.91 (m, 1H), 6.53-6.43 (m, 1H), 4.42-4.29 (m, 1H), 3.82 (s, 2H), 3.57 (s, 2H), 3.45 (s, 2H), 2.66-2.61 (m, 2H), 2.31-2.23 (m, 2H).

### Example 70: N-(2-(4-(2-(2-Aminopyridin-3-yl)-5-phenyl-3H-imidazo[4,5-b]pyridin-3-yl)benzyl)-2-azaspiro[3.3]heptan-6-yl)-4-formyl-3-hydroxybenzamide

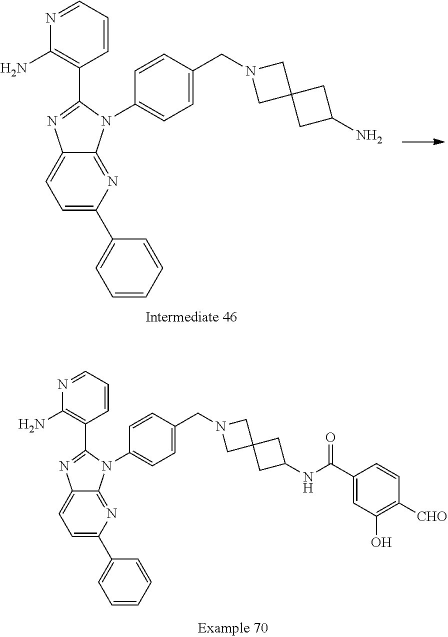 [SMILES: Nc1ncccc1-c1nc2ccc(-c3ccccc3)nc2n1-c1ccc(CN2CC3(CC(N)C3)C2)cc1 ; Nc1ncccc1-c1nc2ccc(-c3ccccc3)nc2n1-c1ccc(CN2CC3(CC(NC(=O)c4ccc(C=O)c(O)c4)C3)C2)cc1]

Following the general procedures described in Example 36, the reaction was carried out using 4-formyl-3-hydroxybenzoic acid and Intermediate 46. After purified by prep-HPLC (column: Phenomenex C18 150×25 mm×10 μm; mobile phase: [water (NH₄HCO₃)-ACN]; gradient: 35%-65% B over 10 min), N-(2-(4-(2-(2-aminopyridin-3-yl)-5-phenyl-3H-imidazo[4,5-b]pyridin-3-yl)benzyl)-2-azaspiro[3.3]heptan-6-yl)-4-formyl-3-hydroxybenzamide (Example 70, 34.6 mg, yield: 18.8%) was obtained as a yellow lyophilized powder. MS: m/z=636.3 [M+H]⁺. ¹H NMR (400 MHz, Methanol-d₄) δ 10.12 (s, 1H), 8.25-8.18 (m, 1H), 8.05-7.99 (m, 3H), 7.96 (d, J=8.4 Hz, 1H), 7.79 (d, J=8.0 Hz, 1H), 7.66-7.58 (m, 4H), 7.44-7.35 (m, 6H), 6.53-6.50 (m, 1H), 4.38-4.31 (m, 3H), 4.15 (s, 2H), 4.04 (s, 2H), 2.77-2.71 (m, 2H), 2.45-2.37 (m, 2H).

### Example 71: 4-(7-(4-(2-(2-Aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde

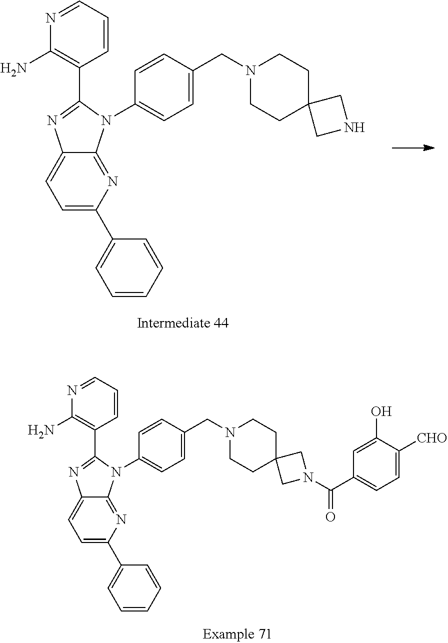 [SMILES: Nc1ncccc1-c1nc2ccc(-c3ccccc3)nc2n1-c1ccc(CN2CCC3(CC2)CNC3)cc1 ; Nc1ncccc1-c1nc2ccc(-c3ccccc3)nc2n1-c1ccc(CN2CCC3(CC2)CN(C(=O)c2ccc(C=O)c(O)c2)C3)cc1]

Following the general procedures described in Example 36, the reaction was carried out using 4-formyl-3-hydroxybenzoic acid and Intermediate 44. After purified by prep-HPLC (column: Phenomenex C18 150×25 mm×10 μm; mobile phase: [water (NH₃H₂O)-ACN]; gradient: 39%-69%, over 12 min), 4-(7-(4-(2-(2-aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde (Example 71, 14.1 mg, yield: 10%) was obtained as a light yellow solid. MS: m/z=650.4 [M+H]⁺. ¹H NMR (400 MHz, Dimethylsulfoxide-d₆) δ 11.27-10.58 (m, 1H), 10.31 (s, 1H), 8.27 (d, J=8.4 Hz, 1H), 8.02 (d, J=7.2 Hz, 2H), 8.00-7.97 (m, 2H), 7.68 (d, J=8.0 Hz, 1H), 7.48-7.44 (m, 6H), 7.40 (d, J=7.2 Hz, 1H), 7.24-7.22 (m, 1H), 7.17-7.13 (m, 2H), 7.03 (br s, 2H), 6.35 (dd, J=7.6, 4.8 Hz, 1H), 3.96 (s, 2H), 3.74 (s, 2H), 3.54 (s, 2H), 2.42-2.34 (m, 2H), 1.94-1.25 (m, 6H).

### Example 72: 4-(4-(4-(2-(2-Aminopyridin-3-yl)-5-phenyl-3H-imidazo[4,5-b]pyridin-3-yl)benzyl)-1,4-diazepane-1-carbonyl)-2-hydroxybenzaldehyde

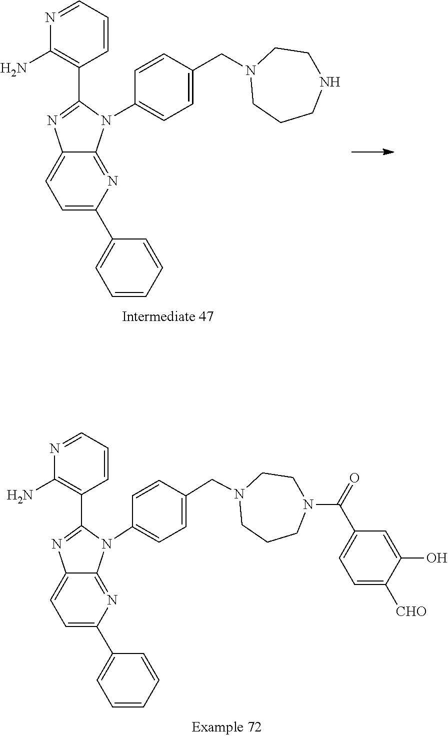 [SMILES: Nc1ncccc1-c1nc2ccc(-c3ccccc3)nc2n1-c1ccc(CN2CCCNCC2)cc1 ; Nc1ncccc1-c1nc2ccc(-c3ccccc3)nc2n1-c1ccc(CN2CCCN(C(=O)c3ccc(C=O)c(O)c3)CC2)cc1]

Following the general procedures described in Example 36, the reaction was carried out using 4-formyl-3-hydroxybenzoic acid and Intermediate 47. After purified by prep-HPLC (column: Phenomenex C18 150×25 mm×10 μm; mobile phase: [water (NH₃H₂O)-ACN]; gradient: 41%-71%, over 12 min), 4-(4-(4-(2-(2-aminopyridin-3-yl)-5-phenyl-3H-imidazo[4,5-b]pyridin-3-yl)benzyl)-1,4-diazepane-1-carbonyl)-2-hydroxybenzaldehyde (Example 72, 24.8 mg, yield: 18%) was obtained as a light yellow solid. MS: m/z=624.4 [M+H]⁺. ¹H NMR (400 MHz, Dimethylsulfoxide-d₆) δ 11.42-10.66 (m, 1H), 10.27 (d, J=11.6 Hz, 1H), 8.27 (dd, J=8.4, 3.6 Hz, 1H), 8.03-8.01 (m, 1H), 8.01-7.95 (m, 3H), 7.71-7.65 (m, 1H), 7.54-7.51 (m, 1H), 7.48-7.41 (m, 6H), 7.17-7.10 (m, 1H), 7.04 (d, J=5.2 Hz, 2H), 6.95-6.93 (m, 1H), 6.91 (d, J=8.0 Hz, 1H), 6.40-6.29 (m, 1H), 3.77 (s, 1H), 3.71 (s, 1H), 3.67-3.63 (m, 2H), 3.36 (s, 2H), 2.71-2.62 (m, 4H), 1.87-1.72 (m, 2H).

### Example 73: 4-(7-(4-(2-(2-Aminopyridin-3-yl)-5-phenyl-3H-imidazo[4,5-b]pyridin-3-yl)benzyl)-2,7-diazaspiro[3.5]nonan-2-yl)-2-hydroxybenzaldehyde

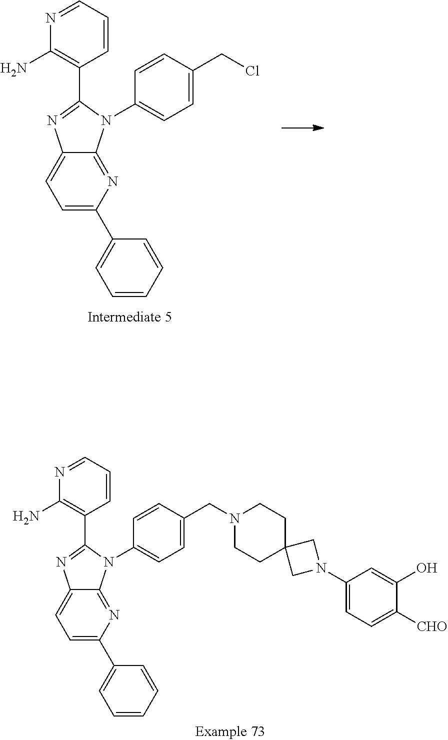 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCC3(CC2)CN(c2ccc(C=O)c(O)c2)C3)cc1]

To a solution of Intermediate 5 (150 mg, 364 μmol) and Intermediate 48 (103 mg, 364 μmol, HCl salt) in DMF (2 mL) were added K₂CO₃ (151 mg, 1.09 mmol) and NaI (5.46 mg, 36.4 μmol). The mixture was stirred at 80 °C for 2 hr. The reaction mixture was filtered and concentrated under reduced pressure. After purified by prep-HPLC (column: Waters xbridge 150×25 mm×10 μm; mobile phase: [water (NH₄HCO₃)-ACN]; gradient: 43%-83% B over 18 min), 4-(7-(4-(2-(2-aminopyridin-3-yl)-5-phenyl-3H-imidazo[4,5-b]pyridin-3-yl)benzyl)-2,7-diazaspiro[3.5]nonan-2-yl)-2-hydroxybenzaldehyde (Example 73, 10.8 mg, yield: 4.5%) was obtained as a light yellow solid. MS: m/z=622.4 [M+H]⁺. ¹H NMR (400 MHz, Dimethylsulfoxide-d4) δ 11.50-11.16 (m, 1H), 9.64 (s, 1H), 8.27 (d, J=8.4 Hz, 1H), 8.05-7.99 (m, 4H), 7.49-7.43 (m, 7H), 7.40-7.39 (m, 1H), 7.18-7.14 (m, 1H), 7.03 (s, 2H), 6.41-6.36 (m, 1H), 6.06-6.02 (m, 1H), 5.77-5.74 (m, 1H), 3.70 (s, 4H), 3.56 (s, 2H), 2.42-2.34 (m, 4H), 1.81-1.77 (m, 4H).

### Example 74: 5-(8-(4-(2-(2-Aminopyridin-3-yl)-5-phenyl-3H-imidazo[4,5-b]pyridin-3-yl)benzyl)-2,8-diazaspiro[4.5]decane-2-carbon 1-2-hydroxybenzaldehyde

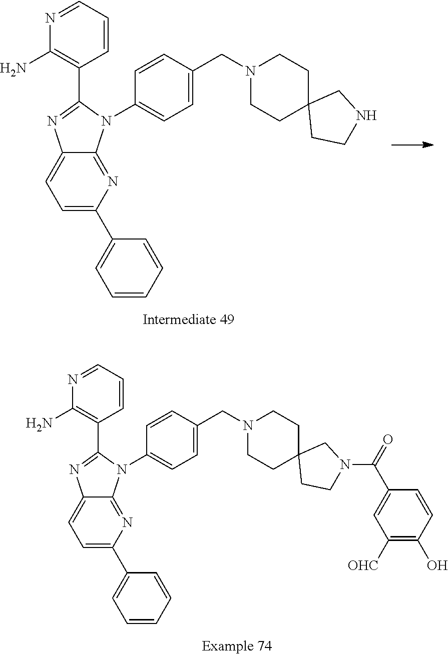 [SMILES: Nc1ncccc1-c1nc2ccc(-c3ccccc3)nc2n1-c1ccc(CN2CCC3(CCNC3)CC2)cc1 ; Nc1ncccc1-c1nc2ccc(-c3ccccc3)nc2n1-c1ccc(CN2CCC3(CC2)CCN(C(=O)c2ccc(O)c(C=O)c2)C3)cc1]

Following the general procedures described in Example 36, the reaction was carried out using 3-formyl-4-hydroxybenzoic acid and Intermediate 49. After purified by prep-HPLC (column: Phenomenex C18 150×25 mm×10 μm; mobile phase: [water (NH₄HCO₃)-ACN]; gradient: 30%-60% B over 14 min), 5-(8-(4-(2-(2-aminopyridin-3-yl)-5-phenyl-3H-imidazo[4,5-b]pyridin-3-yl)benzyl)-2,8-diazaspiro[4.5]decane-2-carbonyl)-2-hydroxybenzaldehyde (Example 74, 8.5 mg, yield: 8%) was obtained as a yellow lyophilized powder. MS: m/z=664.5 [M+H]⁺. ¹H NMR (400 MHz, Dimethylsulfoxide-d₆) δ 10.26 (s, 1H), 8.26 (d, J=8.4 Hz, 1H), 8.08-7.95 (m, 4H), 7.89-7.75 (m, 1H), 7.75-7.60 (m, 1H), 7.50-7.37 (m, 7H), 7.18-7.10 (m, 1H), 7.09-6.92 (m, 3H), 6.52-6.23 (m, 1H), 3.67-3.42 (m, 6H), 2.44-2.34 (m, 2H), 2.33-2.02 (m, 2H), 1.82-1.69 (m, 2H), 1.63-1.46 (m, 4H).

### Example 75: 4-(8-(4-(2-(2-Aminopyridin-3-yl)-5-phenyl-3H-imidazo[4,5-b]pyridin-3-yl)benzyl)-2,8-diazaspiro[4.5]decane-2-carbonyl)-2-hydroxybenzaldehyde

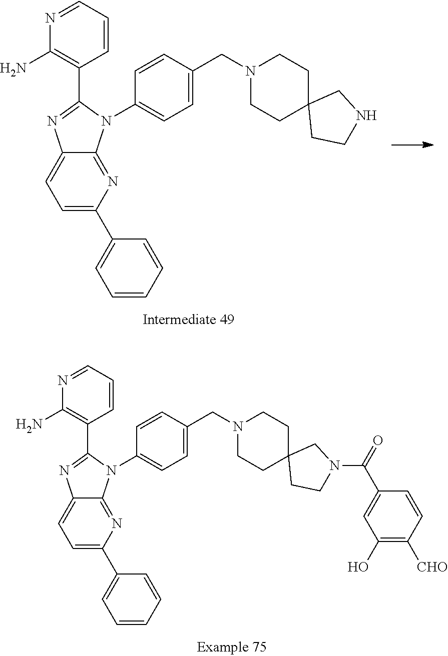 [SMILES: Nc1ncccc1-c1nc2ccc(-c3ccccc3)nc2n1-c1ccc(CN2CCC3(CCNC3)CC2)cc1 ; Nc1ncccc1-c1nc2ccc(-c3ccccc3)nc2n1-c1ccc(CN2CCC3(CC2)CCN(C(=O)c2ccc(C=O)c(O)c2)C3)cc1]

Following the general procedures described in Example 36, the reaction was carried out using 4-formyl-3-hydroxybenzoic acid and Intermediate 49. After purified by prep-HPLC (column: Phenomenex C18 150×25 mm×10 μm; mobile phase: [water (NH₄HCO₃)-ACN]; gradient: 33%-63% B over 14 min), 4-(8-(4-(2-(2-aminopyridin-3-yl)-5-phenyl-3H-imidazo[4,5-b]pyridin-3-yl)benzyl)-2,8-diazaspiro[4.5]decane-2-carbonyl)-2-hydroxybenzaldehyde (Example 75, 28.6 mg, yield: 37%) was obtained as a yellow lyophilized powder. MS: m/z=664.4 [M+H]⁺. ¹H NMR (400 MHz, Dimethylsulfoxide-d₆) S 10.29 (s, 1H), 8.27 (dd, J=8.4, 3.6 Hz, 1H), 8.06-7.94 (m, 4H), 7.77-7.58 (m, 1H), 7.49-7.38 (m, 7H), 7.19-7.11 (m, 1H), 7.08-6.96 (m, 4H), 6.43-6.30 (m, 1H), 3.57-3.50 (m, 2H), 3.44-3.38 (m, 2H), 3.34-3.33 (m, 2H), 2.47-2.37 (m, 2H), 2.36-2.15 (m, 2H), 1.81-1.70 (m, 2H), 1.65-1.54 (m, 2H), 1.53-1.47 (m, 2H).

### Example 76: 4-(8-(4-(2-(2-Aminopyridin-3-yl)-5-phenyl-3H-imidazo[4,5-b]pyridin-3-yl)benzyl)-2,8-diazaspiro[4.5]decan-2-yl)-2-hydroxybenzaldehyde

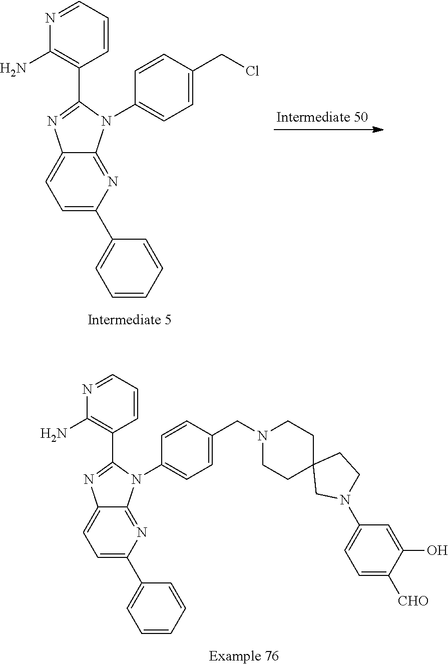 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCC3(CC2)CCN(c2ccc(C=O)c(O)c2)C3)cc1]

Following the general procedures described in Example 73, the reaction was carried out using Intermediate 5 and Intermediate 50. After purified by prep-HPLC (column: Phenomenex C18 150×25 mm×10 μm; mobile phase: [water(NH₄HCO₃)-ACN]; gradient: 53%-83% B over 14 min), 4-(8-(4-(2-(2-aminopyridin-3-yl)-5-phenyl-3H-imidazo[4,5-b]pyridin-3-yl)benzyl)-2,8-diazaspiro[4.5]decan-2-yl)-2-hydroxybenzaldehyde (Example 76, 21.1 mg, yield: 9% for two steps) was obtained as a yellow lyophilized powder. MS: m/z=636.4 [M+H]⁺. ¹H NMR (400 MHz, Dimethylsulfoxide-d_{f};) δ 11.43-11.19 (m, 1H), 9.62 (s, 1H), 8.27 (d, J=8.4 Hz, 1H), 8.05-7.96 (m, 4H), 7.50-7.38 (m, 8H), 7.15 (dd, J=7.6, 1.6 Hz, 1H), 7.03 (s, 2H), 6.37 (dd, J=7.6, 4.8 Hz, 1H), 6.27-6.19 (m, 1H), 5.93-5.87 (m, 1H), 3.59 (s, 2H), 3.44-3.38 (m, 3H), 3.22 (s, 2H), 2.55-2.53 (m, 1H), 2.44-2.36 (m, 2H), 1.89-1.82 (m, 2H), 1.64-1.56 (m, 4H).

### Example 77: 5-(9-(4-(2-(2-Aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde

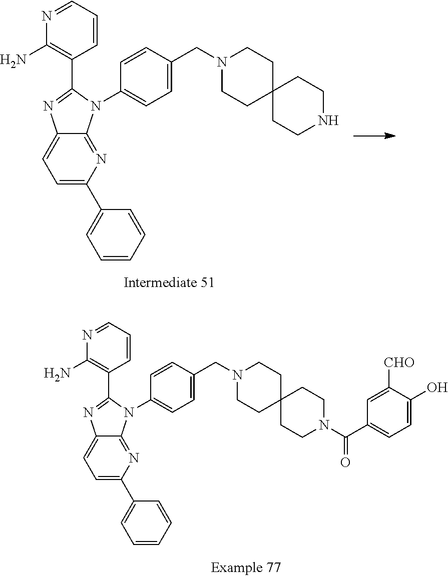 [SMILES: Nc1ncccc1-c1nc2ccc(-c3ccccc3)nc2n1-c1ccc(CN2CCC3(CCNCC3)CC2)cc1 ; Nc1ncccc1-c1nc2ccc(-c3ccccc3)nc2n1-c1ccc(CN2CCC3(CC2)CCN(C(=O)c2ccc(O)c(C=O)c2)CC3)cc1]

Following the general procedures described in Example 36, the reaction was carried out using 4-formyl-3-hydroxybenzoic acid and Intermediate 51. After purified by prep-HPLC (column: Phenomenex C18 150×25 mm×10 μm; mobile phase: [water(NH₄HCO₃)-ACN]; gradient: 33%-63% B over 14 min), 5-(9-(4-(2-(2-aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde (Example 77, 16.5 mg, yield: 16%) was obtained as a yellow lyophilized powder. MS: m/z=678.5 [M+H]⁺. ¹H NMR (400 MHz, Dimethylsulfoxide-d₆) S 10.27 (s, 1H), 8.27 (d, J=8.4 Hz, 1H), 8.07-7.95 (m, 4H), 7.66 (d, J=2.0 Hz, 1H), 7.55 (dd, J=8.4, 2.0 Hz, 1H), 7.49-7.42 (m, 6H), 7.41-7.36 (m, 1H), 7.19-7.12 (m, 1H), 7.10-6.90 (m, 3H), 6.35 (dd, J=7.6, 4.8 Hz, 1H), 3.58 (s, 2H), 3.53-3.41 (m, 4H), 2.47-2.33 (m, 4H), 1.58-1.49 (m, 4H), 1.48-1.37 (m, 4H).

### Example 78: 4-(9-(4-(2-(2-Aminopyridin-3-yl)-5-phenyl-3H-imidazo[4,5-b]pyridin-3-yl)benzyl)-3,9-diazaspiro[5.5]undecan-3-yl)-2-hydroxybenzaldehyde

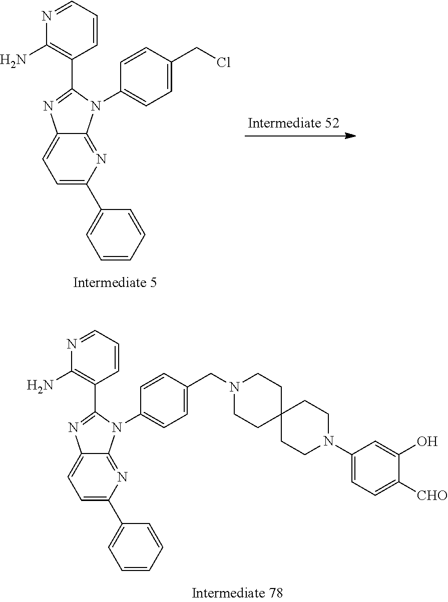 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCC3(CC2)CCN(c2ccc(C=O)c(O)c2)CC3)cc1]

Following the general procedures described in Example 73, the reaction was carried out using Intermediate 5 and Intermediate 52. After purified by prep-HPLC (column: Phenomenex C18 150×25 mm×10 μm; mobile phase: [water (NH₄HCO₃)-ACN]; gradient: 53%-83% B over 14 min), 4-(9-(4-(2-(2-aminopyridin-3-yl)-5-phenyl-3H-imidazo[4,5-b]pyridin-3-yl)benzyl)-3,9-diazaspiro[5.5]undecan-3-yl)-2-hydroxybenzaldehyde (Example 78, 24.3 mg, yield: 10% for two steps) was obtained as a yellow lyophilized powder. MS: m/z=650.2 [M+H]⁺. ¹H NMR (400 MHz, Dimethylsulfoxide-d₆) δ 11.30-10.98 (m, 1H), 9.67 (s, 1H), 8.27 (d, J=8.4 Hz, 1H), 8.05-7.96 (m, 4H), 7.50-7.38 (m, 8H), 7.15 (dd, J=7.6, 1.6 Hz, 1H), 7.03 (s, 2H), 6.37 (d, J=6.8 Hz, 1H), 6.37-6.36 (m, 1H), 6.28-6.27 (m, 1H), 3.59 (s, 2H), 3.44-3.38 (m, 4H), 2.55-2.53 (m, 1H), 2.49-2.40 (m, 3H), 1.64-1.43 (m, 8H).

### Example 79: 4-(6-(4-(2-(2-Aminopyridin-3-yl)-5-phenyl-3H-imidazo[4,5-b]pyridin-3-yl)benzyl)-2,6-diazaspiro[3.4]octan-2-yl)-2-hydroxybenzaldehyde

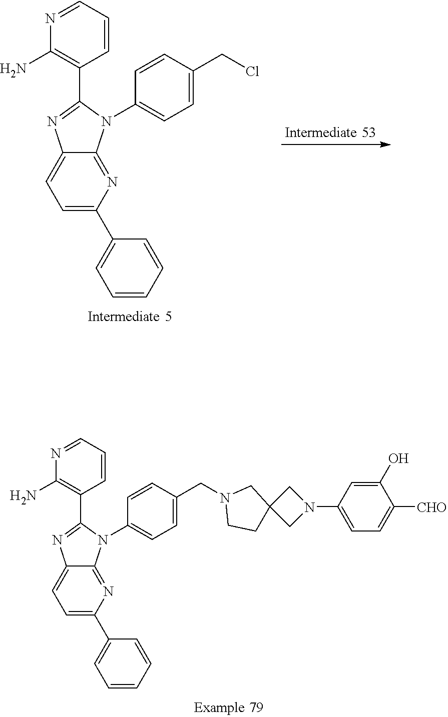

Following the general procedures described in Example 73, the reaction was carried out using Intermediate 5 and Intermediate 53. After purified by silica gel flash chromatography (Eluent of 0%~8% MeOH in CH₂Cl₂), 4-(6-(4-(2-(2-aminopyridin-3-yl)-5-phenyl-3H-imidazo[4,5-b]pyridin-3-yl)benzyl)-2,6-diazaspiro[3.4]octan-2-yl)-2-hydroxybenzaldehyde (Example 79, 12.5 mg, yield: 8% for two steps) was obtained as an off white solid. MS: m/z=608.3 [M+H]⁺. ¹H NMR (400 MHz, Dimethylsulfoxide-d₆) δ 11.31 (s, 1H), 9.66 (s, 1H), 8.27 (d, J=8.4 Hz, 1H), 8.02-7.95 (m, 4H), 7.52-7.48 (m, 2H), 7.46-7.42 (m, 5H), 7.39-7.34 (m, 1H), 7.16 (dd, J=7.6, 1.8 Hz, 1H), 7.03 (s, 2H), 6.36 (dd, J=7.6, 4.8 Hz, 1H), 6.03 (dd, J=8.8, 1.6 Hz, 1H), 5.77 (d, J=1.6 Hz, 1H), 3.92 (s, 4H), 3.70 (s, 2H), 2.76 (s, 2H), 2.62-2.58 (m, 2H), 2.13-2.08 (m, 2H).

### Example 80: 5-(7-(4-(2-(2-Aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde

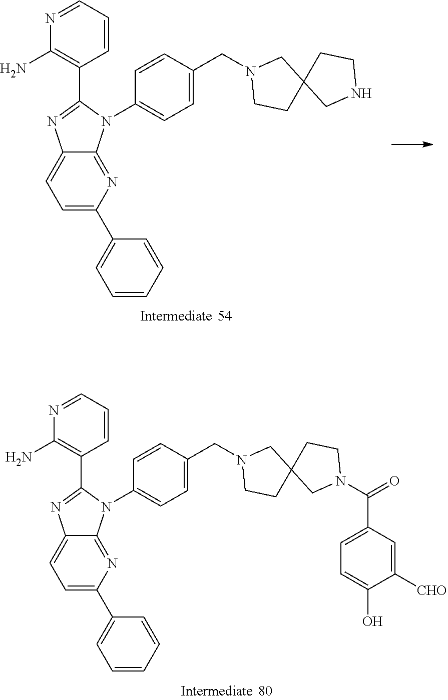

Following the general procedures described in Example 36, the reaction was carried out using 3-formyl-4-hydroxybenzoic acid and Intermediate 54. After purified by prep-HPLC (column: Waters xbridge 150×25 mm 10 μm; mobile phase: [water (NH₄HCO₃)-ACN]; gradient: 38%-68% B over 10 min), 5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 80, 31 mg, yield: 24%) was obtained as a light yellow solid. MS: m/z=650.4 [M+H]⁺. ¹H NMR (400 MHz, Dimethylsulfoxide-d₆) δ 10.26 (s, 1H), 8.26 (d, J=8.4 Hz, 1H), 8.05-7.95 (m, 4H), 7.85-7.77 (m, 1H), 7.73-7.63 (m, 1H), 7.54-7.35 (m, 8H), 7.17-7.10 (m, 1H), 7.05-6.97 (m, 3H), 6.38-6.34 (m, 1H), 3.74-3.60 (m, 2H), 3.50-3.42 (m, 4H), 2.68-2.58 (m, 2H), 2.45-2.32 (m, 2H) 1.92-1.85 (m, 2H), 1.78-1.65 (m, 2H).

### Example 81: 4-(7-(4-(2-(2-Aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde

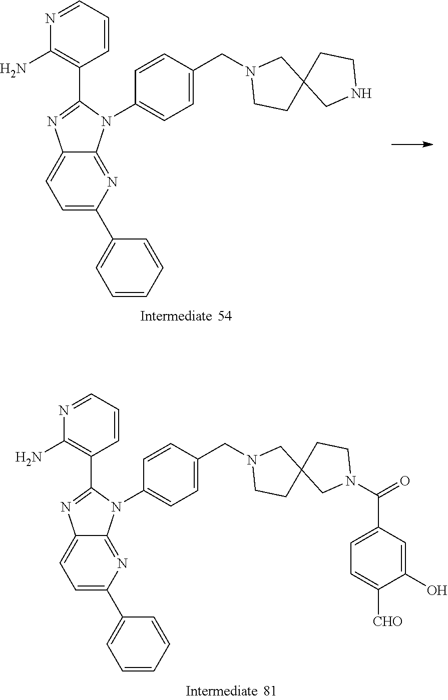

Following the general procedures described in Example 36, the reaction was carried out using 3-formyl-4-hydroxybenzoic acid and Intermediate 54. After purified by prep-HPLC (column: Waters xbridge 150×25 mm×10 μm; mobile phase: [water (NH₄HCO₃)-ACN]; gradient: 40%-70% B over 10 min), 4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 81, 28 mg, yield: 22%) was obtained as a light yellow solid. MS: m/z=650.4 [M+H]⁺. ¹H NMR (400 MHz, Dimethylsulfoxide-d₆) δ 10.27 (s, 1H), 8.28-8.24 (m, 1H), 8.05-7.95 (m, 4H), 7.70-7.65 (m, 1H), 7.54-7.35 (m, 8H), 7.17-7.10 (m, 1H), 7.05-6.97 (m, 31H), 6.95-6.85 (m, 1H), 6.38-6.34 (m, 1H), 3.74-3.60 (m, 2H), 3.50-3.42 (m, 4H), 2.68-2.58 (m, 2H), 2.45-2.32 (m, 2H) 1.90-1.65 (m, 4H).

### Example 82: 4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-2-hydroxybenzaldehyde

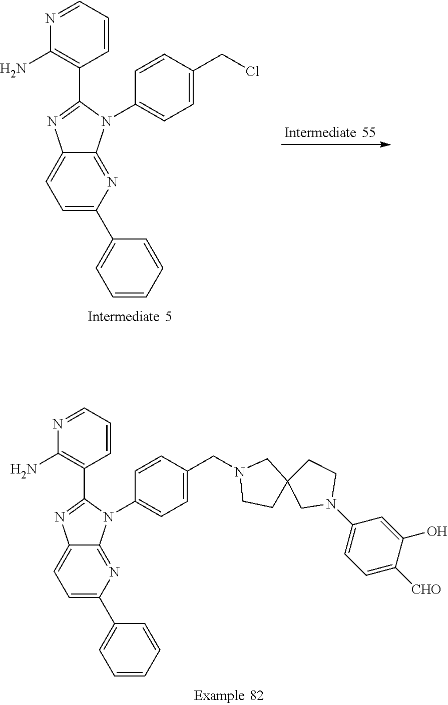

Following the general procedures described in Example 73, the reaction was carried out using Intermediate 5 and Intermediate 55. After purified by prep-HPLC (column: Waters xbridge 150×25 mm 10 μm; mobile phase: [water (NH₄HCO₃)-ACN]; gradient: 55%-85% B over 10 min), 4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde (Example 82, 23.2 mg, yield: 26%) was obtained as a yellow solid. MS: m/z=622.5 [M+H]⁺. ¹H NMR (400 MHz, Dimethylsulfoxide-d₆) δ 11.3 (br s, 1H), 9.63 (s, 1H), 8.26 (d, J=8.4 Hz, 1H), 8.04-7.96 (m, 3H), 7.92 (dd, J=4.8, 1.6 Hz, 1H), 7.52-7.35 (m, 8H), 7.11 (dd, J=7.6, 1.6 Hz, 1H), 7.03 (br s, 2H), 6.30-6.21 (m, 2H), 5.92 (d, J=1.6 Hz, 1H), 3.75-3.65 (m, 2H), 3.42-3.37 (m, 2H), 3.30-3.21 (m, 2H), 2.77-2.68 (m, 1H), 2.63-2.58 (m, 1H), 2.56-2.54 (m, 1H), 2.45-2.40 (m, 1H), 2.03-1.92 (m, 2H), 1.86-1.77 (m, 2H).

### Example 83: 5-(7-(4-(2-(2-Aminopyridin-3-yl)-5-phenyl-3H-imidazo[4,5-b]pyridin-3-yl)benzyl)-2,7-diazaspiro[4.4]nonan-2-yl)-2-hydroxybenzaldehyde

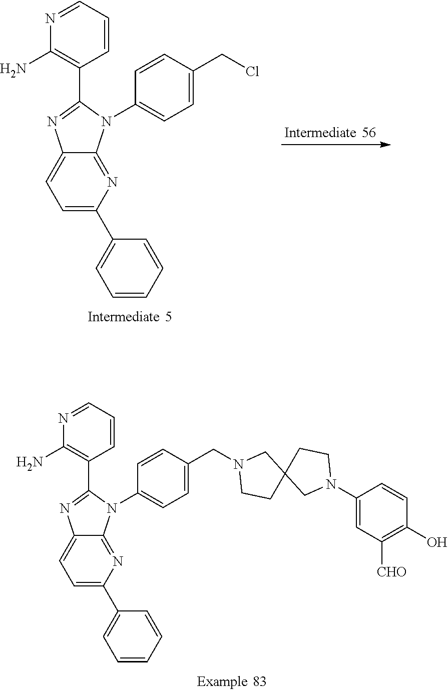

Following the general procedures described in Example 73, the reaction was carried out using Intermediate 5 and Intermediate 56. After purified by prep-TLC (SiO₂, CH₂Cl₂:MeOH=10:1), 5-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde (Example 83, 14.0 mg, yield: 8% for two steps) was obtained as a yellow solid. MS: m/z=622.3 [M+H]⁺. ¹H NMR (400 MHz, Acetonitrile-d₃) δ 9.88 (s, 1H), 8.16 (d, J=8.4 Hz, 1H), 8.02-7.98 (m, 2H), 7.94 (dd, J=4.8, 1.6 Hz, 1H), 7.89 (d, J=8.4 Hz, 1H), 7.52 (d, J=8.4 Hz, 2H), 7.45-7.37 (m, 5H), 7.11 (dd, J=7.6, 1.6 Hz, 1H), 6.78 (d, J=2.8 Hz, 1H), 6.58 (br s, 2H), 6.28 (dd, J=7.6, 4.8 Hz, 1H), 3.78-3.65 (m, 2H), 3.32-3.27 (m, 2H), 3.18 (d, J=8.8 Hz, 1H), 2.93-2.85 (m, 1H), 2.79-2.74 (m, 1H), 2.69-2.56 (m, 2H), 2.51-2.42 (m, 1H), 2.03-2.00 (m, 2H), 1.91-1.87 (m, 2H).

### Example 84: 5-(2-(4-(2-(2-Aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde

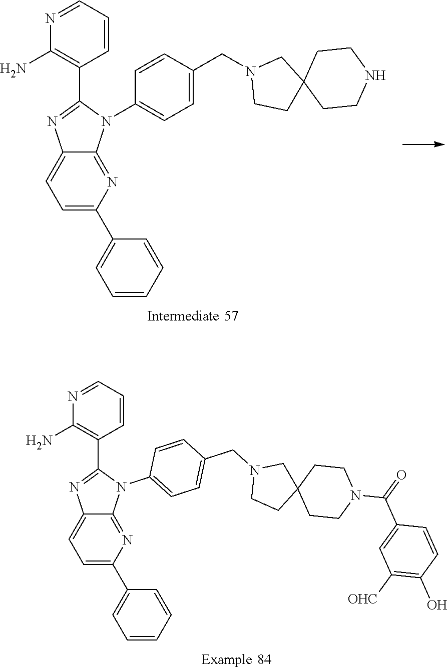

Following the general procedures described in Example 36, the reaction was carried out using 3-formyl-4-hydroxybenzoic acid and Intermediate 57. After purified by prep-HPLC (column: Waters xbridge 150×25 mm×10 μm; mobile phase: [water(NH₄HCO₃)-ACN]; gradient: 38%-68% B over 10 min), 5-(2-(4-(2-(2-aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde (Example 84, 19.3 mg, yield: 17%) was obtained as a light yellow solid. MS: m/z=664.6 [M+H]⁺. ¹H NMR (400 MHz, Dimethylsulfoxide-d₆) δ 10.27 (s, 1H), 8.26 (d, J=8.4 Hz, 1H), 8.05-7.95 (m, 4H), 7.68 (d, J=2.0 Hz, 1H), 7.54 (dd, J=8.8, 2.0 Hz, 1H), 7.48-7.35 (m, 8H), 7.16 (dd, J=6.4, 1.6 Hz, 1H), 7.03-7.00 (m, 2H), 6.38 (dd, J=7.6, 4.8 Hz, 1H), 3.66 (s, 2H), 3.45-3.40 (m, 2H), 3.28-3.21 (m, 2H), 2.61-2.55 (m, 2H), 2.44-2.40 (m, 2H), 1.70-1.63 (m, 2H), 1.58-1.49 (m, 4H).

### Example 85: 4-(2-(4-(2-(2-Aminopyridin-3-yl)-5-phenyl-3H-imidazo[4,5-b]pyridin-3-yl)benzyl)-2,8-diazaspiro[4.5]decan-8-yl)-2-hydroxybenzaldehyde

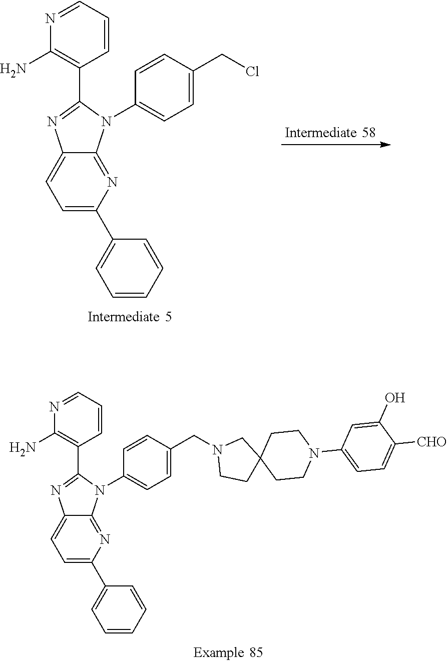

Following the general procedures described in Example 73, the reaction was carried out using Intermediate 5 and Intermediate 58. After purified by prep-TLC (SiO₂, CH₂Cl₂:MeOH=10:1), 4-(2-(4-(2-(2-aminopyridin-3-yl)-5-phenyl-3H-imidazo[4,5-b]pyridin-3-yl)benzyl)-2,8-diazaspiro[4.5]decan-8-yl)-2-hydroxybenzaldehyde (Example 85, 8.7 mg, yield, 4% for two steps) was obtained as a yellow solid. MS: m/z=636.4 [M+H]⁺. ¹H NMR (400 MHz, Dimethylsulfoxide-d₆) δ 11.14 (s, 1H), 9.69 (s, 1H), 8.27 (d, J=8.4 Hz, 1H), 8.05-7.94 (m, 4H), 7.52-7.41 (m, 7H), 7.38-7.35 (m, 1H), 7.16 (d, J=6.8 Hz, 1H), 7.04 (br s, 2H), 6.62-6.56 (m, 1H), 6.35 (dd, J=7.6, 4.8 Hz, 1H), 6.34-6.27 (m, 1H), 3.67 (s, 2H), 3.44-3.35 (m, 4H), 2.63-2.58 (m, 2H), 2.43-2.39 (m, 2H), 1.66 (t, J=6.8 Hz, 2H), 1.65-1.55 (m, 4H).

### Example 86: 5-(2-(4-(2-(2-Aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde

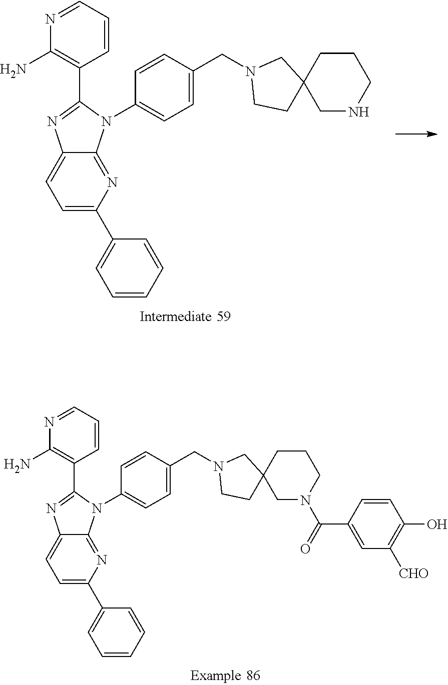

Following the general procedures described in Example 36, the reaction was carried out using 3-formyl-4-hydroxybenzoic acid and Intermediate 59. After purified by prep-HPLC (column: Waters xbridge 150×25 mm 10 μm; mobile phase: [water (NH₄HCO₃)-ACN]; gradient: 42%-72% B over 10 min), 5-(2-(4-(2-(2-aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde (Example 86, 21.8 mg, yield: 32%) was obtained as a yellow solid. MS: m/z=664.4 [M+H]⁺. ¹H NMR (400 MHz, Methanol-d₄)S 10.04 (s, 1H), 8.19 (d, J=8.8 Hz, 1H), 8.03 (d, J=7.6 Hz, 2H), 7.98-7.93 (m, 2H), 7.75 (s, 1H), 7.52 (d, J=8.4 Hz, 2.0 Hz 1H), 7.43-7.31 (m, 8H), 6.97 (d, J=8.0 Hz, 1H), 6.47 (d, J=7.6 Hz, 5.2 Hz, 1H), 3.89-3.54 (m, 6H), 3.48-3.42 (m, 2H), 2.72-2.63 (m, 2H), 1.74-1.59 (m, 6H).

### Example 87: 4-(2-(4-(2-(2-Aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde

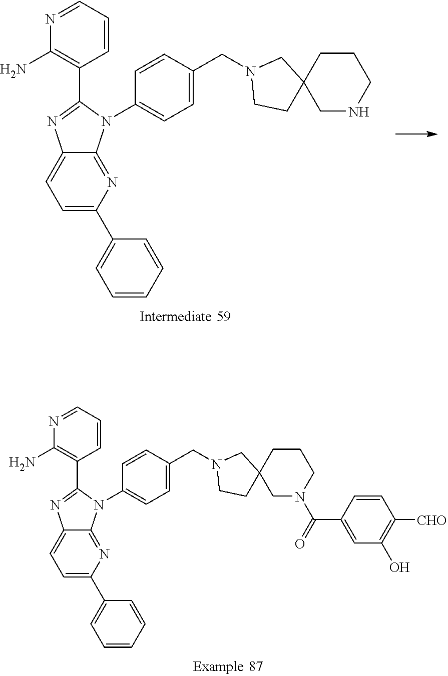

Following the general procedures described in Example 36, the reaction was carried out using 3-formyl-4-hydroxybenzoic acid and Intermediate 59. After purified by prep-HPLC (column: Waters xbridge 150×25 mm 10 μm; mobile phase: [water (NH₄HCO₃)-ACN]; gradient: 45%-75% B over 10 min), 4-(2-(4-(2-(2-aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde (Example 87, 24.8 mg, yield: 37%) was obtained as a yellow solid. MS: m/z=664.4 [M+H]⁺. ¹H NMR (400 MHz, Methanol-d₄) δ 10.08-9.98 (m, 1H), 8.19 (d, J=8.4 Hz, 1H), 8.04-7.94 (m, 4H), 7.75-7.70 (m, 1H), 7.58 (d, J=8.4 Hz, 1H), 7.44-7.33 (m, 7H), 6.93-6.89 (m, 2H), 6.50-6.46 (m, 1H), 3.80-3.75 (m, 1H), 3.66-3.56 (m, 2H), 3.50-3.34 (m, 4H), 2.71-2.62 (m, 2H), 2.44-2.35 (m, 1H), 1.77-1.58 (m, 6H).

### Example 88: 4-(2-(4-(2-(2-Aminopyridin-3-yl)-5-phenyl-3H-imidazo[4,5-b]pyridin-3-yl)benzyl)-2,7-diazaspiro[4.5]decan-7-yl)-2-hydroxybenzaldehyde

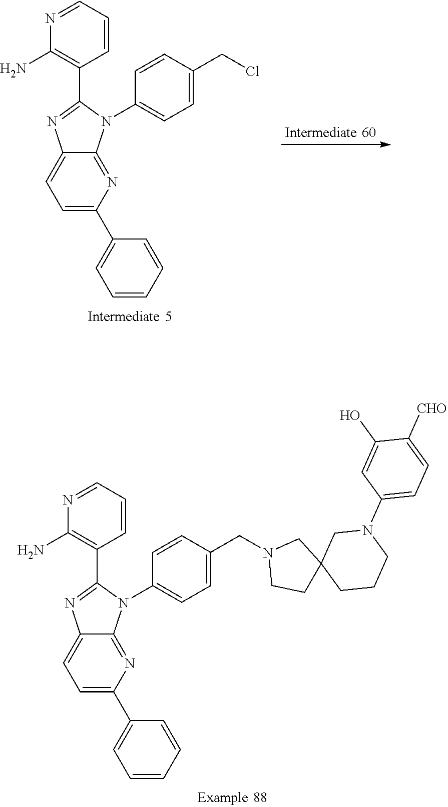

Following the general procedures described in Example 73, the reaction was carried out using Intermediate 5 and Intermediate 60. After purified by prep-HPLC (column: Waters xbridge 150×25 mm 10 μm; mobile phase: [water (NH₄HCO₃)-ACN]; gradient: 53%-83% B over 14 min), 4-(2-(4-(2-(2-aminopyridin-3-yl)-5-phenyl-3H-imidazo[4,5-b]pyridin-3-yl)benzyl)-2,7-diazaspiro[4.5]decan-7-yl)-2-hydroxybenzaldehyde (Example 88, 9.6 mg, yield: 4% for two steps) was obtained as an off white solid. MS: m/z=636.4 [M+H]⁺, ¹H NMR (400 MHz, Methanol-d₄) δ 9.32 (s, 1H), 8.19 (d, J=8.4 Hz, 1H), 8.02 (d, J=6.8 Hz, 2H), 7.97-7.88 (m, 2H), 7.54 (d, J=8.4 Hz, 3H), 7.47-7.32 (m, 5H), 7.30-7.20 (m, 2H), 6.50 (dd, J=8.8, 2.0 Hz, 1H), 6.37 (dd, J=7.6, 5.2 Hz, 1H), 6.27 (d, J=2.4 Hz, 1H), 3.90-3.80 (m, 1H), 3.71-3.64 (m, 1H), 3.59-3.45 (m, 2H), 3.29-3.10 (m, 4H), 3.05-2.92 (m, 1H), 2.77 (d, J=9.6 Hz, 1H), 2.63-2.52 (m, 1H), 2.18 (d, J=9.2 Hz, 1H), 1.75-1.62 (m, 4H).

### Example 89: 4-(6-(4-(2-(2-Aminopyridin-3-yl)-5-phenyl-3H-imidazo[4,5-b]pyridin-3-yl)benzyl)-2,6-diazaspiro[3.3]heptan-2-1-2-hydroxybenzaldehyde

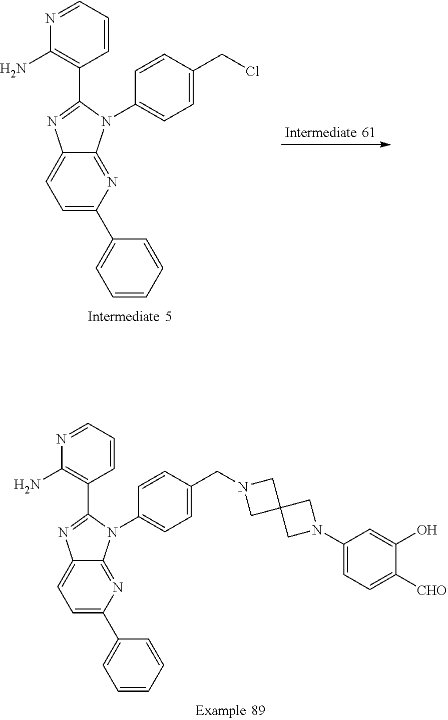

Following the general procedures described in Example 73, the reaction was carried out using Intermediate 5 and Intermediate 61. After purified by silica gel flash chromatography (Eluent of 0%~8% MeOH in CH₂Cl₂), 4-(6-(4-(2-(2-aminopyridin-3-yl)-5-phenyl-3H-imidazo[4,5-b]pyridin-3-yl)benzyl)-2,6-diazaspiro[3.3]heptan-2-yl)-2-hydroxybenzaldehyde (Example 89, 13.1 mg, yield: 9% for two steps) was obtained as a yellow solid. MS: m/z=594.2 [M+H]⁺. ¹H NMR (400 MHz, Dimethylsulfoxide-d₆) δ 9.66 (s, 1H), 8.27 (d, J=8.4 Hz, 1H), 8.04-7.97 (m, 4H), 7.49-7.37 (m, 9H), 7.15 (dd, J=7.6, 1.6 Hz, 1H), 6.99 (s, 2H), 6.40 (dd, J=7.6, 4.8 Hz, 1H), 6.02 (dd, J=8.8, 2.0 Hz, 1H), 5.76 (d, J=2.0 Hz, 1H), 4.08 (s, 4H), 3.64 (s, 2H), 2.07 (s, 4H).

### Example 90: 4-(2-(4-(2-(2-Aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde

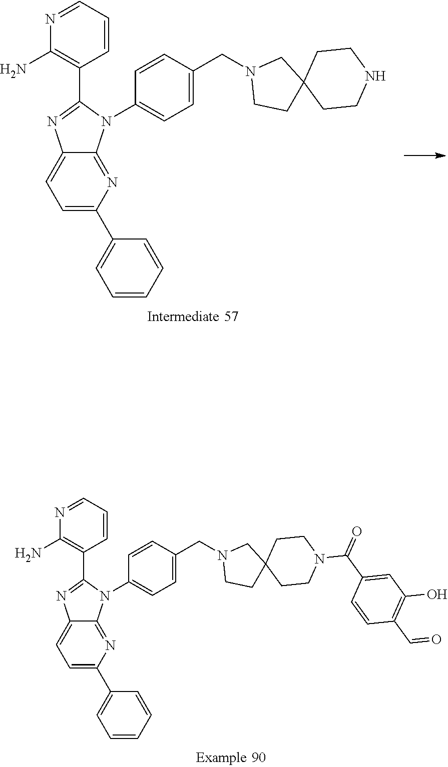

Following the general procedures described in Example 36, the reaction was carried out using 3-formyl-4-hydroxybenzoic acid and Intermediate 57. After purified by prep-HPLC (column: Waters xbridge 150×25 mm×10 μm; mobile phase: [water (NH₄HCO₃)-ACN]; gradient: 38%-68% B over 10 min), 4-(2-(4-(2-(2-aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde (Example 90, 40.6 mg, yield: 33%) was obtained as a light yellow solid. MS: m/z=664.5 [M+H]⁺. ¹H NMR (400 MHz, Dimethylsulfoxide-de) δ 10.28 (s, 1H), 8.28 (d, J=8.4 Hz, 1H), 8.05-7.95 (m, 4H), 7.68 (d, J=8.0 Hz, 1H), 7.48-7.35 (m, 8H), 7.16 (dd, J=7.6, 1.6 Hz, 1H), 7.02 (br s, 2H), 6.94-6.87 (m, 2H), 6.38 (dd, J=7.6, 4.8 Hz, 1H), 3.66-3.61 (m, 3H), 3.53-3.48 (m, 1H), 3.25-3.21 (m, 2H), 2.61-2.55 (m, 2H), 2.44-2.40 (m, 2H), 1.70-1.45 (m, 6H).

### Example 91: 5-(6-(4-(2-(2-Aminopyridin-3-yl)-5-phenyl-3H-imidazo[4,5-b]pyridin-3-yl)benzyl)-2,6-diazaspiro[3.3]heptan-2-yl)-2-hydroxybenzaldehyde

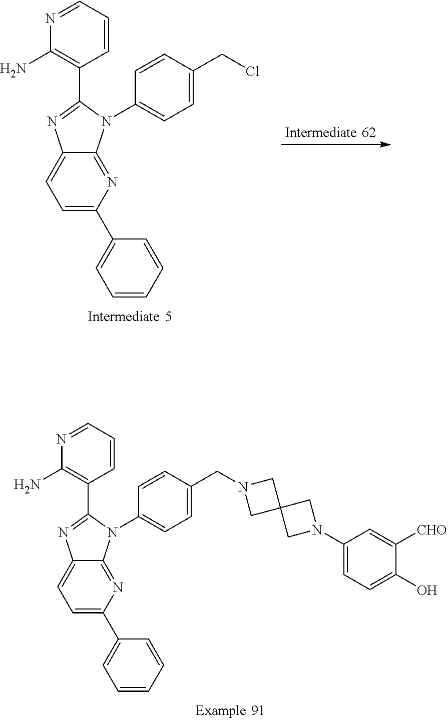

Following the general procedures described in Example 73, the reaction was carried out using Intermediate 5 and Intermediate 62. After purified by silica gel flash chromatography (Eluent of 0%~8% MeOH in CH₂Cl₂), 5-(6-(4-(2-(2-aminopyridin-3-yl)-5-phenyl-3H-imidazo[4,5-b]pyridin-3-yl)benzyl)-2,6-diazaspiro[3.3]heptan-2-yl)-2-hydroxybenzaldehyde (Example 91, 12.5 mg, yield: 9% for two steps) was obtained as a yellow solid. MS: m/z=594.2 [M+H]⁺. ¹H NMR (400 MHz, Dimethylsulfoxide-d₆) δ 11.85 (s, 1H), 8.26 (d, J=8.4 Hz, 1H), 8.07-7.94 (m, 4H), 7.49-7.34 (m, 9H), 7.15 (dd, J=8.0, 2.0 Hz, 1H), 7.00 (s, 2H), 6.93-6.81 (m, 2H), 6.40 (dd, J=7.6, 4.8 Hz, 1H), 4.55 (s, 2H), 4.19 (s, 2H), 3.62 (s, 2H), 3.37 (s, 4H).

### Example 92: 5-(2-(4-(2-(2-Aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde

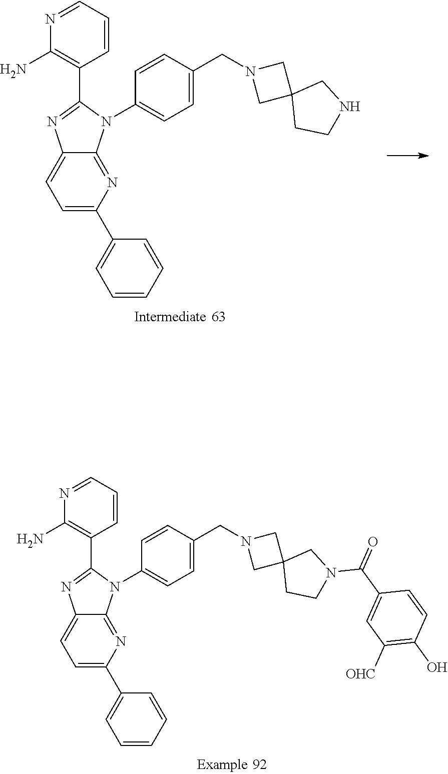

Following the general procedures described in Example 36, the reaction was carried out using 3-formyl-4-hydroxybenzoic acid and Intermediate 63. After purified by prep-HPLC (column: Waters xbridge 150×25 mm 10 μm; mobile phase: [water (NH₄HCO₃)-ACN]; gradient: 29%-59% B over 18 min), 5-(2-(4-(2-(2-aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde (Example 92, 7.5 mg, yield: 5%) was obtained as a yellow solid. MS: m/z=636.4 [M+H]⁺. ¹H NMR (400 MHz, Methanol-d₄)S 10.16-10.03 (m, 1H), 8.19 (d, J=7.2 Hz, 1H), 8.05-7.93 (m, 5H), 7.79-7.70 (m, 1H), 7.50-7.35 (m, 8H), 7.06-6.94 (m, 1H), 6.54-6.41 (m, 1H), 3.84-3.79 (m, 1H), 3.75 (s, 2H), 3.54-3.52 (m, 2H), 3.49-3.45 (m, 3H), 3.36-3.34 (m, 2H), 2.22-2.13 (m, 2H).

### Example 93: 4-(2-(4-(2-(2-Aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde

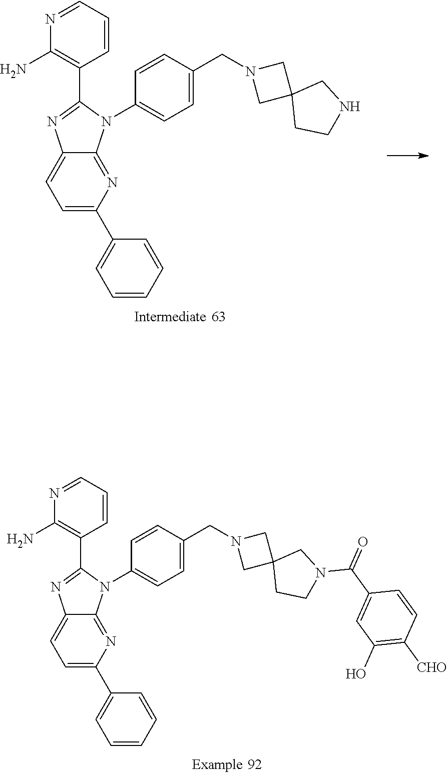

Following the general procedures described in Example 36, the reaction was carried out using 4-formyl-3-hydroxybenzoic acid and Intermediate 63. After purified by prep-HPLC (column: Waters Xbridge C18 150×50 mm×10 μm; mobile phase: [water(NH₄HCO₃)-ACN]; gradient: 41%-71% B over 10 min), 4-(2-(4-(2-(2-aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde (Example 93, 11.8 mg, yield: 17%) was obtained as a yellow solid. MS: m/z=636.4 [M+H]⁺. ¹H NMR (400 MHz, Methanol-d₄)S 10.12 (d, J=5.2 Hz, 1H), 8.20 (dd, J=8.4 Hz, 4.0 Hz, 1H), 8.04-7.94 (m, 5H), 7.81-7.77 (m, 1H), 7.53-7.51 (m, 1H), 7.47-7.40 (m, 6H), 7.11-7.04 (m, 2H), 6.51-6.44 (m, 1H), 3.81 (s, 1H), 3.73 (d, J=3.6 Hz, 2H), 3.63-3.61 (m, 3H), 3.48-3.45 (m, 2H), 3.42 (s, 2H), 2.20-2.15 (m, 2H).

### Example 94: 4-(2-(4-(2-(2-Aminopyridin-3-yl)-5-phenyl-3H-imidazo[4,5-b]pyridin-3-yl)benzyl)-2,6-diazaspiro[3.4]octan-6-yl)-2-hydroxybenzaldehyde

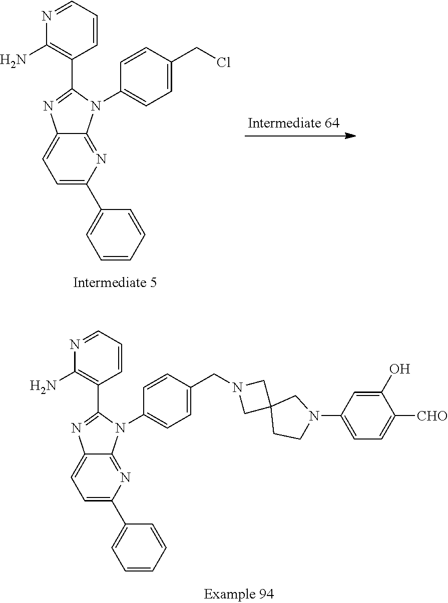 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC3(CCN(c4ccc(C=O)c(O)c4)C3)C2)cc1]

Following the general procedures described in Example 73, the reaction was carried out using Intermediate 5 and Intermediate 64. After purified by prep-HPLC (column: Phenomenex C18 150×25 mm×10 μm; mobile phase: [water (NH₄HCO₃)-ACN]; gradient: 42%-72% B over 14 min), 4-(2-(4-(2-(2-aminopyridin-3-yl)-5-phenyl-3H-imidazo[4,5-b]pyridin-3-yl)benzyl)-2,6-diazaspiro[3.4]octan-6-yl)-2-hydroxybenzaldehyde (Example 94, 13.6 mg, yield: 6% for two steps) was obtained as a yellow solid. MS: m/z=608.2 [M+H]⁺. ¹H NMR (400 MHz, Dimethylsulfoxide-d₆) δ 11.32 (s, 1H), 9.63 (s, 1H), 8.26 (d, J=8.4 Hz, 1H), 8.04-7.96 (m, 4H), 7.48-7.42 (m, 7H), 7.41-7.36 (m, 1H), 7.20-7.13 (m, 1H), 6.99 (s, 2H), 6.39 (dd, J=7.6, 4.8 Hz, 1H), 6.27-6.19 (m, 1H), 5.95-5.89 (m, 1H), 3.70 (s, 2H), 3.50 (s, 2H), 3.37-3.36 (m, 2H), 3.26-3.20 (m, 4H), 2.18-2.15 (m, 2H).

### Example 95: 5-(2-(4-(2-(2-Aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde

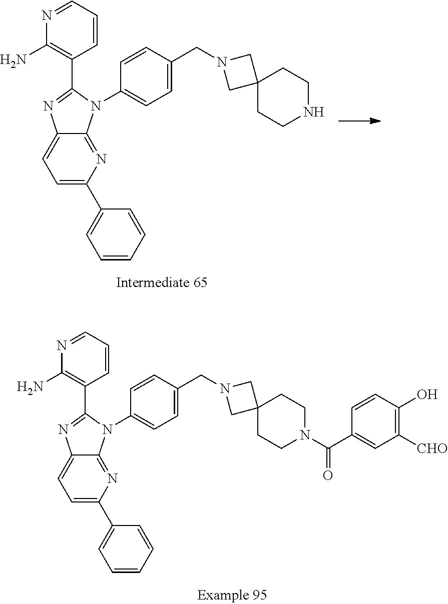 [SMILES: Nc1ncccc1-c1nc2ccc(-c3ccccc3)nc2n1-c1ccc(CN2CC3(CCNCC3)C2)cc1 ; Nc1ncccc1-c1nc2ccc(-c3ccccc3)nc2n1-c1ccc(CN2CC3(CCN(C(=O)c4ccc(O)c(C=O)c4)CC3)C2)cc1]

Following the general procedures described in Example 44, the reaction was carried out using 3-formyl-4-hydroxybenzoic acid and Intermediate 65. After purified by prep-HPLC (column: Phenomenex C18 80×40 mm×3 μm; mobile phase: [water (NH₃H₂O+NH₄HCO₃)-ACN]; gradient: 40%-70% B over 7 min), 5-(2-(4-(2-(2-aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde (Example 95, 18.5 mg, yield: 9.5%) was obtained as yellow solid. MS: m/z=650.3 [M+H]⁺. ¹H NMR (400 MHz, Dimethylsulfoxide-d₆) δ 10.28 (s, 1H), 8.27 (d, J=8.4 Hz, 1H), 8.10-7.93 (m, 4H), 7.65 (d, J=2.4 Hz, 1H), 7.60-7.35 (m, 8H), 7.25-7.12 (m, 1H), 7.08-6.94 (m, 3H), 6.40 (dd, J=7.6, 4.4 Hz, 1H), 3.71 (s, 2H), 3.40-3.36 (m, 4H), 3.07 (s, 4H), 1.85-1.51 (m, 4H).

### Example 96: 4-(2-(4-(2-(2-Aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde

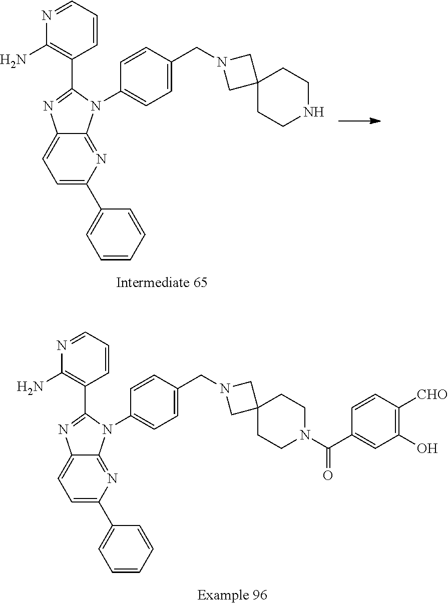 [SMILES: Nc1ncccc1-c1nc2ccc(-c3ccccc3)nc2n1-c1ccc(CN2CC3(CCNCC3)C2)cc1 ; Nc1ncccc1-c1nc2ccc(-c3ccccc3)nc2n1-c1ccc(CN2CC3(CCN(C(=O)c4ccc(C=O)c(O)c4)CC3)C2)cc1]

Following the general procedures described in Example 44, the reaction was carried out using 4-formyl-3-hydroxybenzoic acid and Intermediate 65. After purified by prep-HPLC (column: Welch Xtimate C18 150×25 mm×5 μm; mobile phase: [water (NH₃H₂O+NH₄HCO₃)-ACN]; gradient: 45%-75% B over 7 min), 4-(2-(4-(2-(2-aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde (Example 96, 19.2 mg, yield: 9.9%) was obtained as yellow solid. MS: m/z=650.4 [M+H]⁺. ¹H NMR (400 MHz, Dimethylsulfoxide-d₆) δ 10.28 (s, 1H), 8.26 (d, J=8.4 Hz, 1H), 8.09-7.93 (m, 4H), 7.69 (d, J=7.6 Hz, 1H), 7.49-7.37 (m, 8H), 7.21-7.13 (m, 1H), 7.00 (br s, 2H), 6.95-6.85 (m, 2H), 6.39 (dd, J=7.6, 4.8 Hz, 1H), 3.70 (s, 2H), 3.34-3.31 (m, 4H), 3.06 (s, 4H), 1.87-1.56 (m, 4H).

### Example 97: 4-(2-(4-(2-(2-Aminopyridin-3-yl)-5-phenyl-3H-imidazo[4,5-b]pyridin-3-yl)benzyl)-2,7-diazaspiro[3.5]nonan-7-yl)-2-hydroxybenzaldehyde

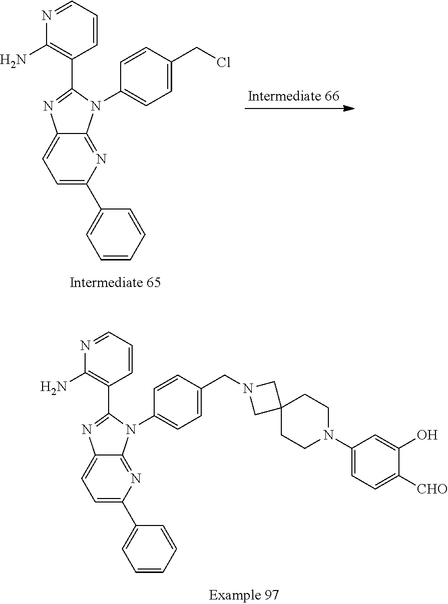 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC3(CCN(c4ccc(C=O)c(O)c4)CC3)C2)cc1]

Following the general procedures described in Example 73, the reaction was carried out using Intermediate 5 and Intermediate 66. After purified by prep-HPLC (column: Waters xbridge 150×25 mm×10 μm; mobile phase: [water (NH₄HCO₃)-ACN]; gradient: 51%-81% B over 14 min), 4-(2-(4-(2-(2-aminopyridin-3-yl)-5-phenyl-3H-imidazo[4,5-b]pyridin-3-yl)benzyl)-2,7-diazaspiro[3.5]nonan-7-yl)-2-hydroxybenzaldehyde (Example 97, 11.6 mg, yield: 7.4%) was obtained as a light yellow solid. MS: m/z=622.5 [M+H]⁺. ¹H NMR (400 MHz, Dimethylsulfoxide-d₆) δ 9.68 (s, 1H), 8.26 (d, J=8.4 Hz, 1H), 8.03-7.97 (m, 4H), 7.48-7.39 (m, 9H), 7.16 (dd, J=7.6, 2.0 Hz, 1H), 6.99 (s, 2H), 6.59 (dd, J=9.2, 2.4 Hz, 1H), 6.40 (dd, J=7.6, 4.8 Hz, 1H), 6.30 (d, J=2.0 Hz, 1H), 3.70 (s, 2H), 3.40-3.37 (m, 4H), 3.05 (s, 4H), 1.77-1.72 (m, 4H).

### Example 98: (R)-5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde

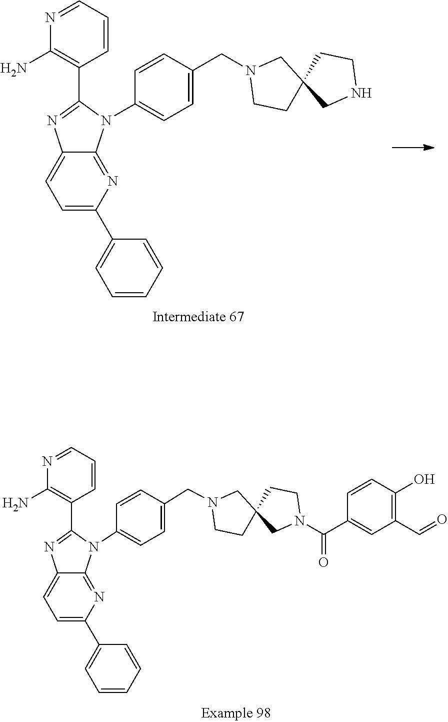 [SMILES: Nc1ncccc1-c1nc2ccc(-c3ccccc3)nc2n1-c1ccc(CN2CC[C@@]3(CCNC3)C2)cc1 ; Nc1ncccc1-c1nc2ccc(-c3ccccc3)nc2n1-c1ccc(CN2CC[C@@]3(CCN(C(=O)c4ccc(O)c(C=O)c4)C3)C2)cc1]

Following the general procedures described in Example 44, the reaction was carried out using 3-formyl-4-hydroxybenzoic acid and Intermediate 67. After purified by prep-HPLC (column: Boston Prime C18 150×30 mm×5 μm; mobile phase: [water (NH₃H₂O+NH₄HCO₃)-ACN]; gradient: 38%-68% B over 7 min), (R)-5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 98, 22.4 mg, 34.5 μmol, yield: 22.4%) was obtained as a yellow solid. MS: m/z=650.4 [M+H]⁺. ¹H NMR (400 MHz, Dimethylsulfoxide-d₆) δ 10.27 (s, 1H), 8.27 (d, J=8.4 Hz, 1H), 8.14-7.95 (m, 5H), 7.80-7.78 (m, 1H), 7.67-7.65 (m, 1H), 7.52-7.41 (m, 7H), 7.19-7.01 (m, 4H), 6.45-6.27 (m, 1H), 4.12-3.51 (m, 2H), 3.50-3.48 (m, 2H), 3.35-3.22 (m, 2H), 2.69-2.55 (m, 2H), 2.50-2.13 (m, 2H), 1.99-1.71 (m, 4H).

### Example 99: (R)-4-(7-(4-(2-(2-aminopyridin-3-yl)-5-phenyl-3H-imidazo[4,5-b]pyridin-3-yl)benzyl 2,7-diazaspiro[4.4]nonane-2-carbon 1-2-hydroxybenzaldehyde

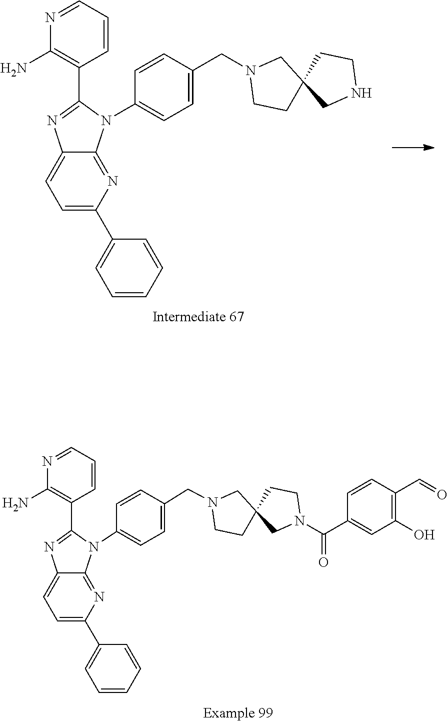 [SMILES: Nc1ncccc1-c1nc2ccc(-c3ccccc3)nc2n1-c1ccc(CN2CC[C@@]3(CCNC3)C2)cc1 ; Nc1ncccc1-c1nc2ccc(-c3ccccc3)nc2n1-c1ccc(CN2CC[C@@]3(CCN(C(=O)c4ccc(C=O)c(O)c4)C3)C2)cc1]

Following the general procedures described in Example 44, the reaction was carried out using 4-formyl-3-hydroxybenzoic acid and Intermediate 67. After purified by prep-HPLC (column: Welch Xtimate C18 150×30 mm×5 μm; mobile phase: [water (NH₃H₂O+NH₄HCO₃)-ACN]; gradient: 55%-85% B over 7 min), (R)-4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 99, 30.6 mg, yield: 31%) was obtained as a yellow solid. MS: m/z=650.4 [M+H]⁺. ¹H NMR (400 MHz, Dimethylsulfoxide-d₆) δ 10.27 (s, 1H), 8.26 (d, J=8.4 Hz, 1H), 8.07-7.96 (m, 4H), 7.73-7.62 (m, 1H), 7.59-7.30 (m, 8H), 7.17-6.92 (m, 5H), 6.45-6.25 (m, 1H), 3.81-3.57 (m, 2H), 3.56-3.45 (m, 2H), 3.28-3.22 (m, 2H), 2.67-2.55 (m, 2H), 2.46-2.25 (m, 2H), 1.92-1.67 (m, 4H).

### Example 100: (R)-4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-2-hydroxybenzaldehyde

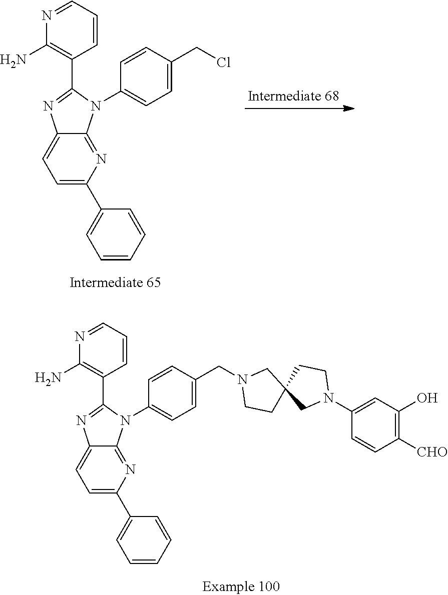 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC[C@@]3(CCN(c4ccc(C=O)c(O)c4)C3)C2)cc1]

Following the general procedures described in Example 73, the reaction was carried out using Intermediate 5 and Intermediate 68. After purified by silica gel flash chromatography (Eluent of 0%~3% MeOH in CH₂Cl₂) and prep-HPLC (column: Welch Xtimate C18 150×25 mm×5 μm; mobile phase: [water(NH₃H₂O+NH₄HCO₃)-ACN]; gradient: 80%-100% B over 7 min), (R)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde (Example 100, 23.3 mg, yield: 4% for two steps) was obtained as a yellow solid. MS: m/z=622.4 [M+H]⁺. ¹H NMR (400 MHz, Dimethylsulfoxide-d₆) δ 11.32 (s, 1H), 9.63 (s, 1H), 8.26 (d, J=8.4 Hz, 1H), 8.05-7.95 (m, 3H), 7.90-7.82 (m, 1H), 7.51-7.30 (m, 8H), 7.11-7.07 (m, 1H), 7.04 (s, 2H), 6.30-6.22 (m, 2H), 5.92 (s, 1H), 3.75-3.64 (m, 2H), 3.40-3.32 (m, 3H), 3.30-3.26 (m, 1H), 2.80-2.72 (m, 1H), 2.62-2.51 (m, 2H), 2.46-2.38 (m, 1H), 2.05-1.92 (m, 2H), 1.90-1.75 (m, 2H).

### Example 101: (S)-5-(7-(4-(2-(2-Aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde

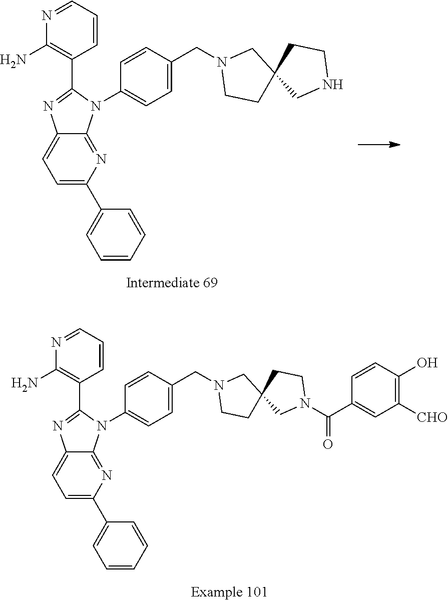 [SMILES: Nc1ncccc1-c1nc2ccc(-c3ccccc3)nc2n1-c1ccc(CN2CC[C@]3(CCNC3)C2)cc1 ; Nc1ncccc1-c1nc2ccc(-c3ccccc3)nc2n1-c1ccc(CN2CC[C@]3(CCN(C(=O)c4ccc(O)c(C=O)c4)C3)C2)cc1]

Following the general procedures described in Example 44, the reaction was carried out using 3-formyl-4-hydroxybenzoic acid and Intermediate 69. After purified by prep-HPLC (column: Welch Xtimate C18 150×25 mm×5 m; mobile phase: [water (NH₃H₂O+NH₄HCO₃)-ACN]; gradient: 40%-70% B over 7 min), (S)-5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 101, 16.0 mg, yield: 13%) was obtained as a yellow solid. MS: m/z=672.3 [M+Na]⁺. ¹H NMR (400 MHz, Dimethylsulfoxide-d₆) δ 10.26 (s, 1H), 8.26 (d, J=8.4 Hz, 1H), 8.07-7.92 (m, 4H), 7.85-7.32 (m, 10H), 7.17-6.98 (m, 4H), 6.37-6.32 (m, 1H), 3.71-3.38 (m, 6H), 2.67-2.55 (m, 2H), 2.47-2.36 (m, 2H), 1.94-1.65 (m, 4H).

### Example 102: (S)-4-(7-(4-(2-(2-Aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde

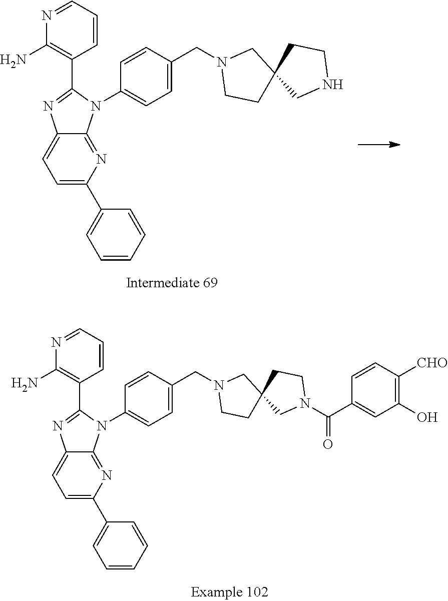 [SMILES: Nc1ncccc1-c1nc2ccc(-c3ccccc3)nc2n1-c1ccc(CN2CC[C@]3(CCNC3)C2)cc1 ; Nc1ncccc1-c1nc2ccc(-c3ccccc3)nc2n1-c1ccc(CN2CC[C@]3(CCN(C(=O)c4ccc(C=O)c(O)c4)C3)C2)cc1]

Following the general procedures described in Example 44, the reaction was carried out using 4-formyl-3-hydroxybenzoic acid and Intermediate 69. After purified by prep-HPLC (column: Welch Xtimate C18 150×25 mm×5 □m; mobile phase: [water (NH₃H₂O+NH₄HCO₃)-ACN]; gradient: 45%-75% B over 7 min), (S)-4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde (Example 102, 31.4 mg, yield: 26%) was obtained as a yellow solid. MS: m/z=650.4 [M+H]⁺. ¹H NMR (400 MHz, Dimethylsulfoxide-d₆) δ 10.29-10.26 (m, 1H), 8.26 (d, J=8.4 Hz, 1H), 8.04-7.96 (m, 4H), 7.69-7.64 (m, 1H), 7.52-7.34 (m, 8H), 7.18-7.10 (m, 1H), 7.06-6.94 (m, 4H), 6.39-6.32 (m, 1H), 3.71-3.40 (m, 2H), 3.27-3.22 (m, 2H), 2.68-2.56 (m, 4H), 2.46-2.31 (m, 2H), 1.94-1.66 (m, 4H).

### Example 103: (S)-4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-2-hydroxybenzaldehyde

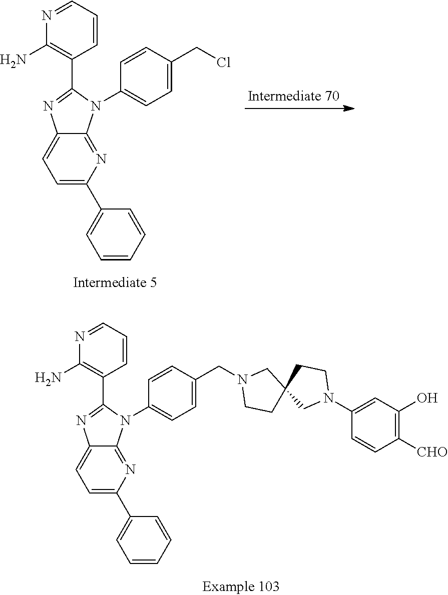 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC[C@]3(CCN(c4ccc(C=O)c(O)c4)C3)C2)cc1]

To a solution of Intermediate 70 (75.5 mg, 267 μmol, HCl) in DMF (0.5 mL) were added Intermediate 5 (100 mg, 243 μmol) and DIEA (126 mg, 971 μmol). The mixture was stirred at 80 °C for 1 hr. The mixture was concentrated under reduced pressure. After purified by prep-HPLC (column: Welch Xtimate C18 150×25 mm×5 μm; mobile phase: [water (NH₃H₂O+NH₄HCO₃)-ACN]; gradient: 80%-100% B over 7 min), (S)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde (Example 103, 15.1 mg, yield: 10%) was obtained as a yellow solid. MS: m/z=622.4 [M+H]⁺. ¹H NMR (400 MHz, Dimethylsulfoxide-d₆) δ 11.34 (s, 1H), 9.64 (s, 1H), 8.27 (d, J=8.0 Hz, 1H), 8.06-7.95 (m, 3H), 7.94-7.92 (m, 1H), 7.59-7.35 (m, 9H), 7.12 (dd, J=7.6, 1.6 Hz, 1H), 7.05-7.02 (m, 1H), 6.31-6.20 (m, 2H), 5.93 (d, J=1.6 Hz, 1H), 3.76-3.63 (m, 2H), 3.46-3.40 (m, 1H), 3.30-3.25 (m, 2H), 2.77-2.63 (m, 4H), 2.46-2.40 (m, 1H), 2.07-1.94 (m, 2H), 1.90-1.76 (m, 2H).

### Example 104: (R)-4-(4-(4-(2-(2-Aminopyridin-3-yl)-5-phenyl-3H-imidazo[4,5-b]pyridin-3-yl)benzyl)-2-methylpiperazin-1-yl)-2-hydroxybenzaldehyde

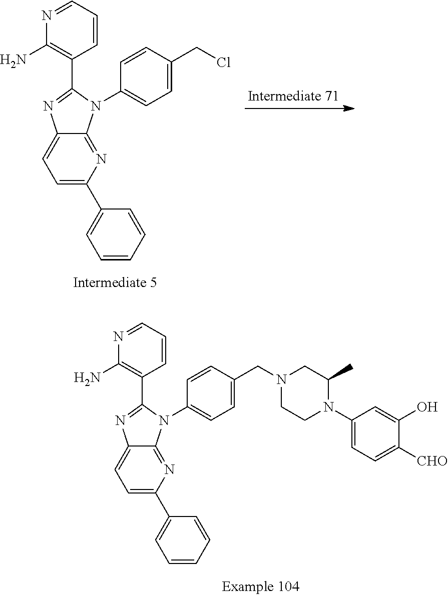 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)CCN1c1ccc(C=O)c(O)c1]

Following the general procedures described in Example 73, the reaction was carried out using Intermediate 5 and Intermediate 71. After purified by prep-HPLC (column: Phenomenex C18 75×30 mm×3 μm; mobile phase: [water(NH₃H₂O+NH₄HCO₃)-ACN]; gradient: 70%-100% B over 8 min), (R)-4-(4-(4-(2-(2-aminopyridin-3-yl)-5-phenyl-3H-imidazo[4,5-b]pyridin-3-yl)benzyl)-2-methylpiperazin-1-yl)-2-hydroxybenzaldehyde (Intermediate 104, 9.1 mg, yield: 4.2% for two steps) was obtained as an off-white solid. MS: m/z=596.3 [M+H]⁺. ¹H NMR (400 MHz, Dimethylsulfoxide-d6) 11.13 (s, 1H), 9.72 (s, 1H), 8.28 (d, J=8.4 Hz, 1H), 8.05-7.98 (m, 4H), 7.59-7.37 (m, 8H), 7.21-7.14 (m, 1H), 7.13-6.97 (m, 2H), 6.65-6.53 (m, 1H), 6.45-6.36 (m, 1H), 6.33-6.24 (m, 1H), 4.33-4.12 (m, 1H), 3.75-3.45 (m, 2H), 3.16-2.70 (m, 4H), 2.27-2.16 (m, 2H), 1.16 (d, J=6.0 Hz, 3H).

### Example 105: (S)-5-(4-(4-(2-(2-Aminopyridin-3-yl)-5-phenyl-3H-imidazo[4,5-b]pyridin-3-yl)benzyl)-2-methylpiperazine-1-carbonyl)-2-hydroxybenzaldehyde

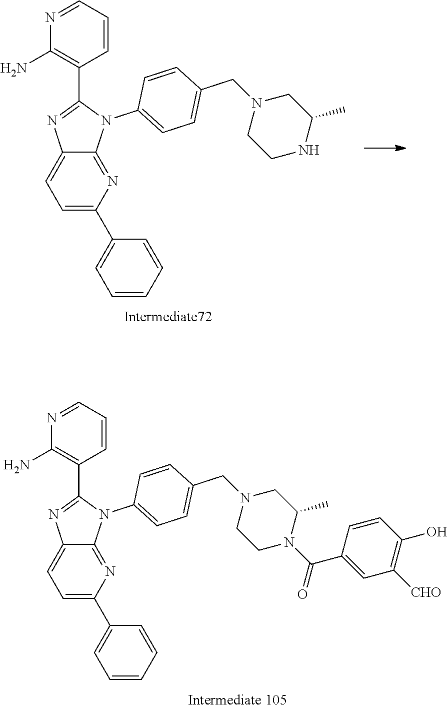 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1C(=O)c1ccc(O)c(C=O)c1]

Following the general procedures described in Example 44, the reaction was carried out using 3-formyl-4-hydroxybenzoic acid and Intermediate 72. After purified by silica gel flash chromatography (Methanol in CH₂Cl₂=0-10%) and prep-HPLC (column: Xtimate C18 150×40 mm×5 μm; mobile phase: [water (HCl)-ACN]; gradient: 15%-45% B over 10 min), (S)-5-(4-(4-(2-(2-aminopyridin-3-yl)-5-phenyl-3H-imidazo[4,5-b]pyridin-3-yl)benzyl)-2-methylpiperazine-1-carbonyl)-2-hydroxybenzaldehyde (Example 105, 5.4 mg, yield: 4.4%) was obtained as a yellow oil. MS: m/z=624.3 [M+H]⁺. ¹H NMR (400 MHz, Dimethylsulfoxide-d₆) δ 11.30 (s, 1H), 11.15-10.97 (m, 1H), 10.31 (s, 1H), 8.51-8.32 (m, 3H), 8.13 (dd, J=6.4, 1.6 Hz, 1H), 8.09-8.04 (m, 3H), 7.90-7.85 (m, 2H), 7.83-7.79 (m, 1H), 7.75-7.71 (m, 1H), 7.70-7.67 (m, 2H), 7.61 (dd, J=8.4, 2.0 Hz, 1H), 7.50-7.45 (m, 2H), 7.44-7.39 (m, 1H), 7.12 (d, J=8.8 Hz, 1H), 6.90-6.83 (m, 1H), 4.49-4.47 (m, 1H), 4.44-4.20 (m, 2H), 3.40-3.21 (m, 4H), 3.18-3.05 (m, 2H), 1.43 (d, J=7.2 Hz, 3H).

### Example 106: (S)-4-(4-(4-(2-(2-Aminopyridin-3-yl)-5-phenyl-3H-imidazo[4,5-b]pyridin-3-yl)benzyl)-2-methylpiperazine-1-carbonyl)-2-hydroxybenzaldehyde

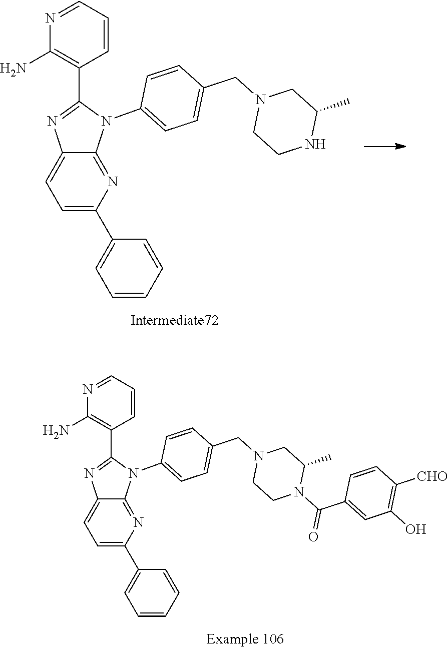

Following the general procedures described in Example 44, the reaction was carried out using 4-formyl-3-hydroxybenzoic acid and Intermediate 72. After purified by silica gel flash chromatography (Methanol in Dichloromethane=0-10%) and prep-HPLC (column: Xtimate C18 150×40 mm×5 μm; mobile phase: [water (HCl)-ACN]; gradient: 15%-45% B over 10 min), (S)-4-(4-(4-(2-(2-aminopyridin-3-yl)-5-phenyl-3H-imidazo[4,5-b]pyridin-3-yl)benzyl)-2-methylpiperazine-1-carbonyl)-2-hydroxybenzaldehyde (Intermediate 106, 10.7 mg, yield: 8.8%) was obtained as a yellow solid. MS: m/z=624.2 [M+H]⁺. ¹H NMR (400 MHz, Dimethylsulfoxide-d₆) δ 11.50-11.08 (m, 2H), 10.31 (s, 1H), 8.78-8.25 (m, 3H), 8.15 (d, J=5.2 Hz, 1H), 8.10-8.01 (m, 3H), 7.95-7.78 (m, 3H), 7.75-7.62 (m, 3H), 7.53-7.39 (m, 3H), 7.15-7.07 (m, 1H), 7.01-6.94 (m, 1H), 6.92-6.81 (m, 1H), 4.57-4.49 (m, 1H), 4.38-4.30 (m, 1H), 3.61-2.90 (m, 7H), 1.45 (d, J=5.6 Hz, 3H).

### Example 107: (S)-4-(4-(4-(2-(2-aminopyridin-3-yl)-5-phenyl-3H-imidazo[4,5-b]pyridin-3-yl)benzyl)-2-methylpiperazin-1-yl)-2-hydroxybenzaldehyde

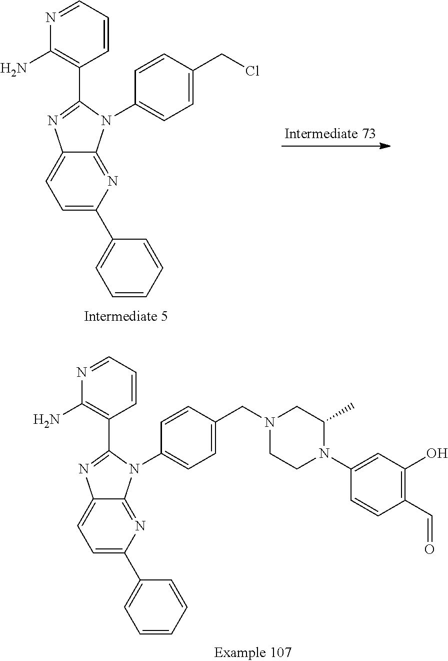 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)CCN1c1ccc(C=O)c(O)c1]

Following the general procedures described in Example 103, the reaction was carried out using Intermediate 5 and Intermediate 73. After purified by prep-HPLC (column: Welch Xtimate C18 150×25 mm×5 μm; mobile phase: [water (NH₃H₂O+NH₄HCO₃)-ACN]; gradient: 80%-100% B over 7 min), (S)-4-(4-(4-(2-(2-aminopyridin-3-yl)-5-phenyl-3H-imidazo[4,5-b]pyridin-3-yl)benzyl)-2-methylpiperazin-1-yl)-2-hydroxybenzaldehyde (Example 107, 42.5 mg, yield: 20%) was obtained as a yellow solid. MS: m/z=596.4 [M+H]⁺. ¹H NMR (400 MHz, Dimethylsulfoxide-d₆) δ 11.13 (s, 1H), 9.71 (s, 1H), 8.27 (d, J=8.4 Hz, 1H), 8.0-7.97 (m, 4H), 7.56-7.36 (m, 8H), 7.16 (dd, J=8.0, 2.0 Hz, 1H), 7.07-7.03 (m, 2H), 6.57 (dd, 1=8.4, 2.0 Hz, 1H), 6.41-6.36 (m, 1H), 6.27 (d, J=4.8 Hz, 1H), 4.27-4.20 (m, 1H), 3.74-3.65 (m, 2H), 3.57-3.51 (m, 1H), 3.18-3.07 (m, 1H), 2.96 (d, J=10.4 Hz, 1H), 2.76-2.65 (m, 1H), 2.35-2.21 (m, 1H), 2.20-2.10 (m, 1H), 1.16 (d, J=7.2 Hz, 3H).

### Example 108: 4-(7-(4-(2-(2-Aminopyridin-3-yl)-5-phenyl-3H-imidazo[4,5-b]pyridin-3-yl)benzyl)-4,7-diazaspiro[2.5]octan-4-yl)-2-hydroxybenzaldehyde

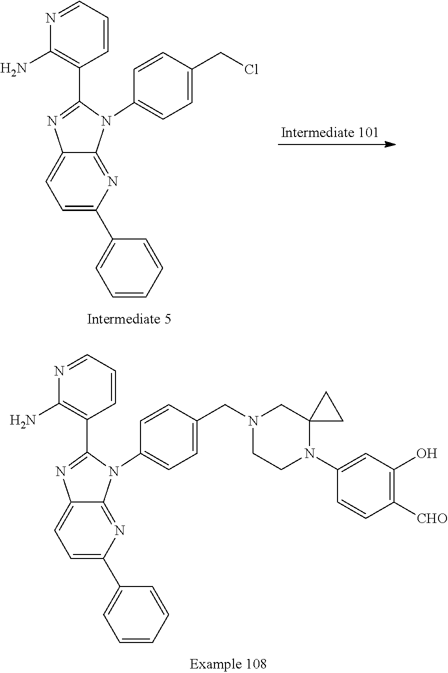 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCN(c3ccc(C=O)c(O)c3)C3(CC3)C2)cc1]

Following the general procedures described in Example 103, the reaction was carried out using Intermediate 5 and Intermediate 101. After purified by prep-HPLC (column: Welch Xtimate C18 150×25 mm×5 m; mobile phase: [water (NH₃H₂O+NH₄HCO₃)-ACN]; gradient: 75%-100% B over 7 min), 4-(7-(4-(2-(2-aminopyridin-3-yl)-5-phenyl-3H-imidazo[4,5-b]pyridin-3-yl)benzyl)-4,7-diazaspiro[2.5]octan-4-yl)-2-hydroxybenzaldehyde (Example 108, 68.5 mg, yield: 37%) was obtained as a white solid. MS: m/z=608.3 [M+H]⁺. ¹H NMR (400 MHz, Dimethylsulfoxide-d₆) δ 11.01 (s, 1H), 9.79 (s, 1H), 8.27 (d, J=8.8 Hz, 1H), 8.07-7.94 (m, 4H), 7.54-7.35 (m, 8H), 7.12-7.03 (m, 3H), 6.67 (dd, J=8.8, 1.6 Hz, 1H), 6.54 (d, J=1.6 Hz, 1H), 6.26 (dd, J=8.0, 4.8 Hz, 1H), 4.13-4.00 (m, 1H), 3.68-3.39 (m, 3H), 2.83-2.61 (m, 2H), 2.17-1.89 (m, 2H), 1.21-1.08 (m, 1H), 1.02-0.90 (m, 1H) 0.83-0.62 (m, 2H).

### Example 109: 4-(3-(4-(2-(2-Aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde

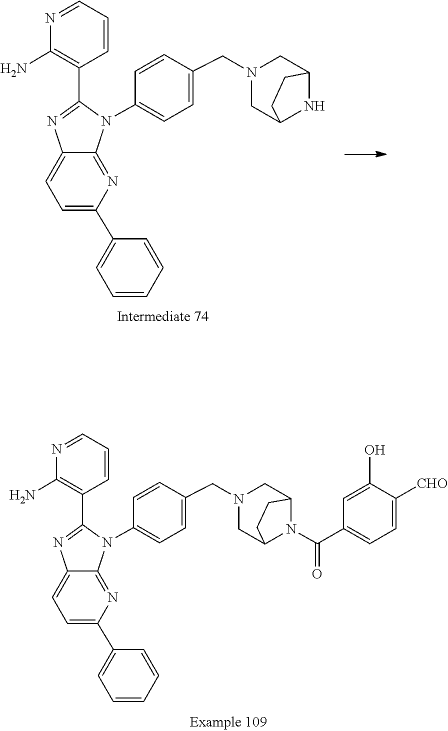 [SMILES: Nc1ncccc1-c1nc2ccc(-c3ccccc3)nc2n1-c1ccc(CN2CC3CCC(C2)N3)cc1 ; Nc1ncccc1-c1nc2ccc(-c3ccccc3)nc2n1-c1ccc(CN2CC3CCC(C2)N3C(=O)c2ccc(C=O)c(O)c2)cc1]

Following the general procedures described in Example 36, the reaction was carried out using 4-formyl-3-hydroxybenzoic acid and Intermediate 74. After purified by prep-HPLC (column: Phenomenex C₁₈ 150×25 mm×10 μm; mobile phase: [water (NH₄HCO₃)-ACN]; gradient: 38%-68%, over 10 min), 4-(3-(4-(2-(2-aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde (Example 109, 7.5 mg, yield: 4%) was obtained as a yellow solid. MS: m/z=636.4 [M+H]⁺. ¹H NMR (400 MHz, Dimethylsulfoxide-d₆) δ 11.47-10.44 (m, 1H), 10.29 (s, 1H), 8.26 (d, J=8.4 Hz, 1H), 8.07-7.96 (m, 4H), 7.69 (d, J=8.0 Hz, 1H), 7.53-7.39 (m, 7H), 7.15-7.11 (m, 1H), 7.06-6.92 (m, 4H), 6.36 (dd, J=7.6, 4.8 Hz, 1H), 4.65-4.53 (m, 1H), 4.01-3.89 (m, 1H), 3.70-3.58 (m, 2H), 2.83-2.71 (m, 1H), 2.67-2.64 (m, 1H), 2.38-2.31 (m, 2H), 1.96-1.80 (m, 4H).

### Example 110: 5-(3-(4-(2-(2-Aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde

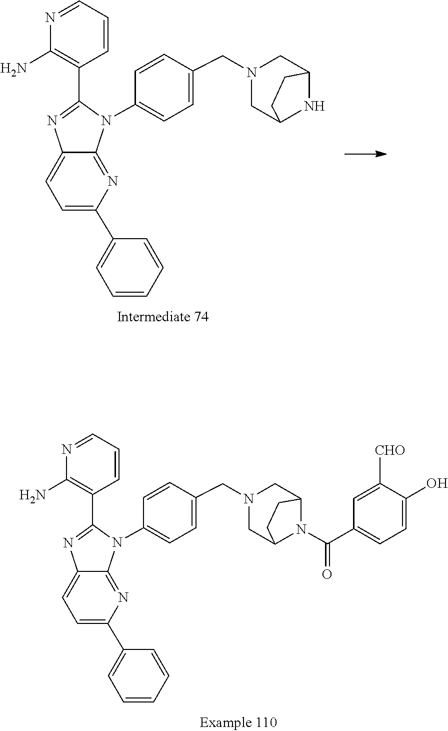 [SMILES: Nc1ncccc1-c1nc2ccc(-c3ccccc3)nc2n1-c1ccc(CN2CC3CCC(C2)N3)cc1 ; Nc1ncccc1-c1nc2ccc(-c3ccccc3)nc2n1-c1ccc(CN2CC3CCC(C2)N3C(=O)c2ccc(O)c(C=O)c2)cc1]

Following the general procedures described in Example 36, the reaction was carried out using 3-formyl-4-hydroxybenzoic acid and Intermediate 74. After purified by prep-HPLC (column: Phenomenex C₁₈ 150×25 mm×10 μm; mobile phase: [water (NH₃H₂O)-ACN]; gradient: 18%-48%, over 16 min), 5-(3-(4-(2-(2-aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde (Example 110, 8.1 mg, yield: 4%) was obtained as a yellow solid. MS: m/z=636.4 [M+H]⁺. ¹H NMR (400 MHz, Dimethylsulfoxide-d₆) δ 10.26 (s, 1H), 8.27 (d, J=8.4 Hz, 1H), 8.06-7.97 (m, 4H), 7.76 (d, J=2.4 Hz, 1H), 7.64-7.57 (m, 1H), 7.53-7.40 (m, 7H), 7.17-7.11 (m, 1H), 7.04 (s, 2H), 6.96 (d, J=8.4 Hz, 1H), 6.41-6.34 (m, 1H), 3.63 (s, 2H), 3.03-2.92 (m, 1H), 2.83-2.82 (m, 1H), 2.75-2.68 (m, 2H), 2.38-2.31 (m, 2H), 1.92-1.74 (m, 4H).

### Example 111: 4-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)-2-hydroxybenzaldehyde

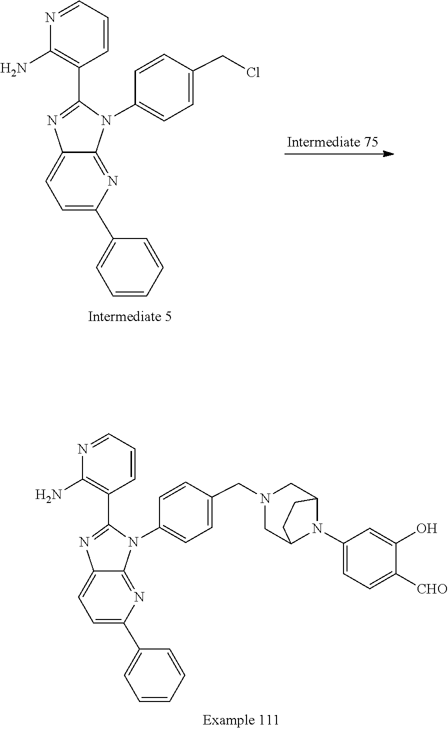 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC3CCC(C2)N3c2ccc(C=O)c(O)c2)cc1]

Following the general procedures described in Example 73, the reaction was carried out using Intermediate 5 and Intermediate 75. After purified by prep-HPLC (column: Waters xbridge 150×25 mm×10 μm; mobile phase: [water (NH₄HCO₃)-ACN]; gradient: 63%-93% B over 10 min), 4-(3-(4-(2-(2-aminopyridin-3-yl)-5-phenyl-3H-imidazo[4,5-b]pyridin-3-yl)benzyl)-3,8-diazabicyclo[3.2.1]octan-8-yl)-2-hydroxybenzaldehyde (Example 111, 17.9 mg, yield: 8%) was obtained as a yellow solid. MS: m/z=608.4 [M+H]⁺. ¹H NMR (400 MHz, Dimethylsulfoxide-d₆) δ 11.3 (s, 1H), 9.64 (s, 1H), 8.27 (d, J=8.4 Hz, 1H), 8.04-7.97 (m, 4H), 7.49-7.40 (m, 8H), 7.12-7.05 (m, 3H), 6.51 (dd, J=8.8, 2.0 Hz, 1H), 6.29 (dd, J=7.6, 4.8 Hz, 1H), 6.25 (d, J=2.0 Hz, 1H), 4.50-4.40 (m, 2H), 3.55 (s, 2H), 2.63-2.58 (m, 2H), 2.35-2.32 (m, 2H), 2.04-1.97 (m, 2H), 1.93-1.85 (m, 2H).

### Example 112: 5-(8-(4-(2-(2-Aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde

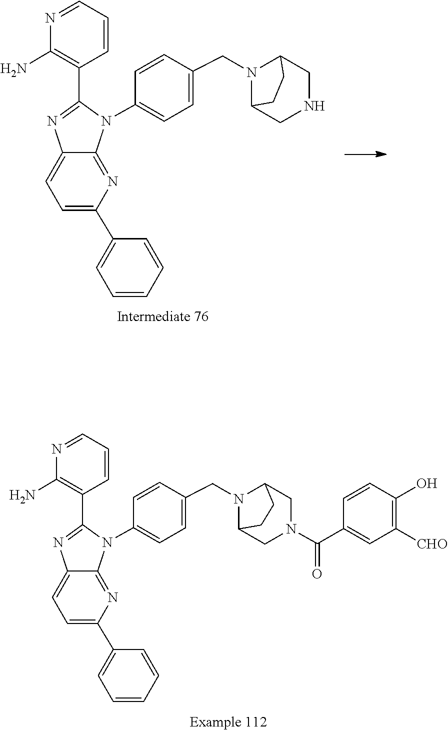 [SMILES: Nc1ncccc1-c1nc2ccc(-c3ccccc3)nc2n1-c1ccc(CN2C3CCC2CNC3)cc1 ; Nc1ncccc1-c1nc2ccc(-c3ccccc3)nc2n1-c1ccc(CN2C3CCC2CN(C(=O)c2ccc(O)c(C=O)c2)C3)cc1]

Following the general procedures described in Example 36, the reaction was carried out using 3-formyl-4-hydroxybenzoic acid and Intermediate 76. After purified by prep-HPLC (column: Waters xbridge 150×25 mm×10 μm; mobile phase: [water (NH₄HCO₃)-ACN]; gradient: 35%-65% B over 10 min), 5-(8-(4-(2-(2-aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde (Example 112, 20.0 mg, yield: 15%) was obtained as a light yellow solid. MS: m/z=636.4 [M+H]⁺. ¹H NMR (400 MHz, Methanol-d₄) δ 10.14 (s, 1H), 8.21 (d, J=8.4 Hz, 1H), 8.05-7.95 (m, 4H), 7.81 (d, J=2.0 Hz, 1H), 7.65-7.60 (m, 3H), 7.48-7.35 (m, 9H), 7.04 (d, J=8.4 Hz, 1H), 6.50 (dd, J=7.6, 5.2 Hz, 1H), 4.63-4.59 (m, 1H), 4.46-4.38 (m, 1H), 3.70 (s, 2H), 3.54-3.45 (m, 2H), 3.20-3.14 (m, 2H), 2.15-2.10 (m, 2H), 1.80-1.70 (m, 2H).

### Example 113: 4-(8-(4-(2-(2-Aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde

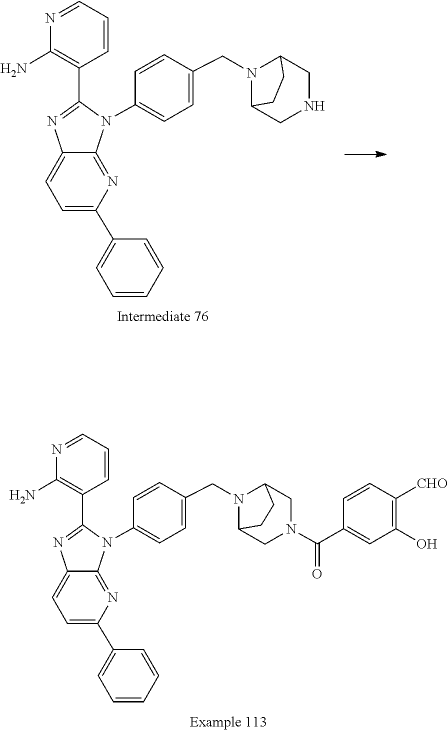 [SMILES: Nc1ncccc1-c1nc2ccc(-c3ccccc3)nc2n1-c1ccc(CN2C3CCC2CNC3)cc1 ; Nc1ncccc1-c1nc2ccc(-c3ccccc3)nc2n1-c1ccc(CN2C3CCC2CN(C(=O)c2ccc(C=O)c(O)c2)C3)cc1]

Following the general procedures described in Example 36, the reaction was carried out using 3-formyl-4-hydroxybenzoic acid and Intermediate 76. After purified by prep-HPLC (column: Waters xbridge 150×25 mm×10 μm; mobile phase: [water (NH₄HCO₃)-ACN]; gradient: 38%-68% B over 10 min), 4-(8-(4-(2-(2-aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde (Example 113, 20.0 mg, yield: 15%) was obtained as a light yellow solid. MS: m/z=636.4 [M+H]⁺. ¹H NMR (400 MHz, Methanol-d₄) δ 10.11 (s, 1H), 8.19 (d, J=8.4 Hz, 1H), 8.04-8.01 (m, 2H), 7.98 (dd, J=4.8, 1.6 Hz, 1H), 7.94 (d, J=8.8 Hz, 1H), 7.80 (d, J=7.6 Hz, 1H), 7.63-7.60 (m, 2H), 7.47-7.36 (m, 6H), 7.34 (dd, J=7.6, 2.0 Hz, 1H), 7.02-6.98 (m, 1H), 6.95 (d, J=1.2 Hz, 1H), 6.48 (dd, J=7.6, 4.8 Hz, 1H), 4.39-4.29 (m, 1H), 3.68 (s, 2H), 3.49-3.45 (m, 1H), 3.40-3.35 (m, 2H), 3.23-3.19 (m, 1H), 3.17-3.12 (m, 1H), 2.18-2.08 (m, 2H), 1.78-1.74 (m, 1H), 1.65-1.58 (m, 1H).

### Example 114: 4-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)-2-hydroxybenzaldehyde

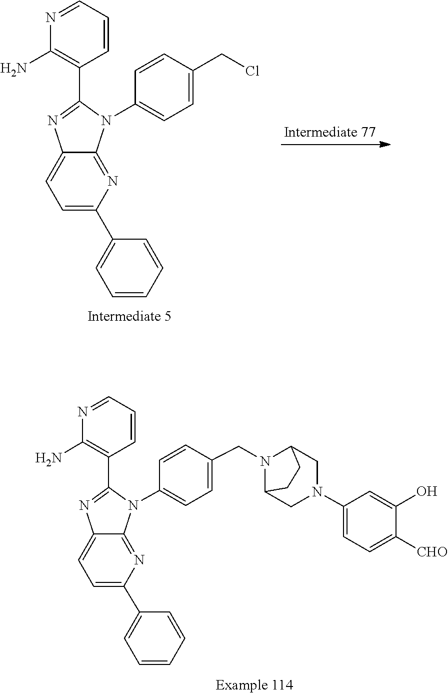 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2C3CCC2CN(c2ccc(C=O)c(O)c2)C3)cc1]

Following the general procedures described in Example 73, the reaction was carried out using Intermediate 5 and Intermediate 77. After purified by prep-HPLC (column: Waters xbridge 150×25 mm×10 μm; mobile phase: [water (NH₄HCO₃)-ACN]; gradient: 20%-50% B over 53 min), 4-(8-(4-(2-(2-aminopyridin-3-yl)-5-phenyl-3H-imidazo[4,5-b]pyridin-3-yl)benzyl)-3,8-diazabicyclo[3.2.1]octan-3-yl)-2-hydroxybenzaldehyde (Example 114, 62.0 mg, yield: 34%) was obtained as a light yellow solid. MS: m/z=608.4 [M+H]⁺. ¹H NMR (400 MHz, Dimethylsulfoxide-d₆) δ 11.30-10.92 (m, 1H), 9.71 (s, 1H), 8.27 (d, J=8.4 Hz, 11H), 8.05-7.95 (m, 4H), 7.58 (d, J=8.4 Hz, 2H), 7.50-7.35 (m, 7H), 7.18 (dd, J=7.6, 1.2 Hz, 1H), 7.01 (br s, 2H), 6.51 (dd, J=9.2, 2.4 Hz, 1H), 6.40 (dd, J=7.6, 4.8 Hz, 1H), 6.21 (d, J=2.4 Hz, 1H), 4.04-4.03 (m, 2H), 3.70 (s, 2H), 3.61-3.56 (m, 2H), 3.09-3.05 (m, 2H), 2.06-2.02 (m, 2H), 1.67-1.60 (m, 2H).

### Example 115: N-(1-(4-(2-(2-Aminopyridin-3-yl)-5-phenyl-3H-imidazo[4,5-b]pyridin-3-yl)benzyl)azepan-4-yl)-3-formyl-4-hydroxybenzamide

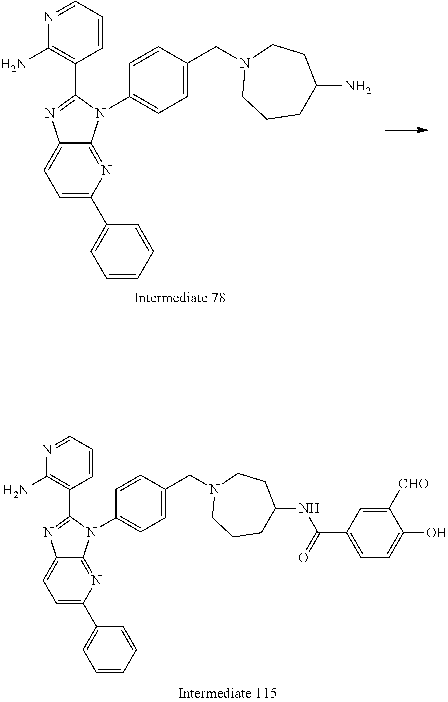 [SMILES: Nc1ncccc1-c1nc2ccc(-c3ccccc3)nc2n1-c1ccc(CN2CCCC(N)CC2)cc1 ; Nc1ncccc1-c1nc2ccc(-c3ccccc3)nc2n1-c1ccc(CN2CCCC(NC(=O)c3ccc(O)c(C=O)c3)CC2)cc1]

Following the general procedures described in Example 36, the reaction was carried out using 3-formyl-4-hydroxybenzoic acid and Intermediate 78. After purified by prep-HPLC (column: Phenomenex Cis 150×25 mm×10 μm; mobile phase: [water(NH₄HCO₃)-ACN]; gradient: 35%-65% B over 10 min), N-(1-(4-(2-(2-aminopyridin-3-yl)-5-phenyl-3H-imidazo[4,5-b]pyridin-3-yl)benzyl)azepan-4-yl)-3-formyl-4-hydroxybenzamide (Example 115, 11.4 mg, yield: 15%) was obtained as a yellow solid. MS: m/z=638.4 [M+H]⁺. ¹H NMR (400 MHz, Dimethylsulfoxide-d₆) δ 10.30 (s, 1H), 8.31 (d, J=7.6 Hz, 1H), 8.26 (d, J=8.4 Hz, 1H), 8.20 (d, J=2.0 Hz, 1H), 8.04-7.96 (m, 5H), 7.55-7.50 (m, 2H), 7.46-7.42 (m, 4H), 7.39-7.35 (m, 1H), 7.17-7.13 (m, 1H), 7.09-6.98 (m, 3H), 6.37 (dd, J=7.6, 4.8 Hz, 1H), 4.15-4.00 (m, 1H), 3.74-3.67 (m, 2H), 2.74-2.64 (m, 4H), 1.90-1.70 (m, 6H).

### Example 116: N-(1-(4-(2-(2-aminopyridin-3-yl)-5-phenyl-3H-imidazo[4,5-b]pyridin-3-yl)benzyl)azepan-4-yl)-4-formyl-3-hydroxybenzamide

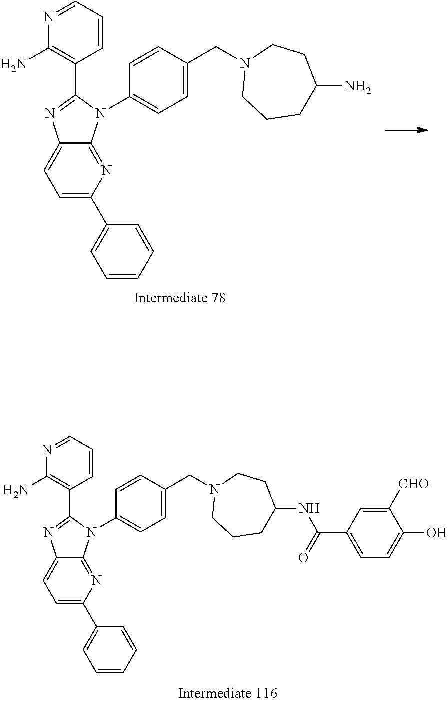 [SMILES: Nc1ncccc1-c1nc2ccc(-c3ccccc3)nc2n1-c1ccc(CN2CCCC(N)CC2)cc1 ; Nc1ncccc1-c1nc2ccc(-c3ccccc3)nc2n1-c1ccc(CN2CCCC(NC(=O)c3ccc(O)c(C=O)c3)CC2)cc1]

Following the general procedures described in Example 36, the reaction was carried out using 4-formyl-3-hydroxybenzoic acid and Intermediate 78. After purified by prep-HPLC (column: Phenomenex C₁₈ 150×25 mm×10 μm; mobile phase: [water(NH₄HCO₃)-ACN]; gradient: 42%-72% B over 10 min), N-(1-(4-(2-(2-aminopyridin-3-yl)-5-phenyl-3H-imidazo[4,5-b]pyridin-3-yl)benzyl)azepan-4-yl)-4-formyl-3-hydroxybenzamide (Example 116, 7.8 mg, yield: 11%) was obtained as a yellow solid. MS: m/z=638.5 [M+H]⁺. ¹H NMR (400 MHz, Dimethylsulfoxide-d₆) δ 10.30 (s, 1H), 8.44 (d, J=8.0 Hz, 1H), 8.27 (d, J=8.4 Hz, 1H), 8.04-7.97 (m, 4H), 7.69 (d, J=8.0 Hz, 1H), 7.55-7.50 (m, 2H), 7.48-7.39 (m, 6H), 7.38-7.33 (m, 2H), 7.16 (dd, J=7.6, 1.6 Hz, 1H), 7.04 (s, 2H), 6.37 (dd, J=7.6, 4.8 Hz, 1H), 4.12-4.04 (m, 1H), 3.33 (s, 2H), 2.70-2.59 (m, 4H), 1.90-1.56 (m, 6H).

### Example 117: N-(1-(4-(2-(2-Aminopyridin-3-yl)-5-phenyl-3H-imidazo[4,5-b]pyridin-3-yl)benzyl)azepan-3-yl)-3-formyl-4-hydroxybenzamide

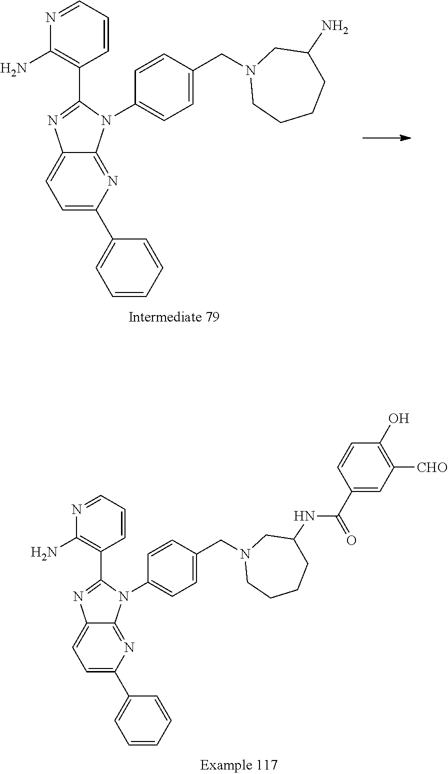 [SMILES: Nc1ncccc1-c1nc2ccc(-c3ccccc3)nc2n1-c1ccc(CN2CCCCC(N)C2)cc1 ; Nc1ncccc1-c1nc2ccc(-c3ccccc3)nc2n1-c1ccc(CN2CCCCC(NC(=O)c3ccc(O)c(C=O)c3)C2)cc1]

Following the general procedures described in Example 36, the reaction was carried out using 3-formyl-4-hydroxybenzoic acid and Intermediate 79. After purified by prep-HPLC (column: Phenomenex C₁₈ 150×25 mm×10 μm; mobile phase: [water (NH₄HCO₃)-ACN]; gradient: 40%-70% B over 14 min), N-(1-(4-(2-(2-aminopyridin-3-yl)-5-phenyl-3H-imidazo[4,5-b]pyridin-3-yl)benzyl)azepan-3-yl)-3-formyl-4-hydroxybenzamide (Example 117, 12.6 mg, yield: 19%) was obtained as a yellow solid. MS: m/z=638.5 [M+H]⁺. ¹H NMR (400 MHz, Dimethylsulfoxide-d₆) δ 10.24 (s, 1H), 8.26 (d, J=8.4 Hz, 1H), 8.19-8.10 (m, 2H), 8.04-7.96 (m, 3H), 7.95-7.88 (m, 2H), 7.55 (d, J=8.4 Hz, 2H), 7.46-7.38 (m, 5H), 7.11-7.02 (m, 3H), 6.95 (d, J=8.4 Hz, 1H), 6.29 (dd, J=7.6, 4.8 Hz, 1H), 4.15-4.05 (m, 1H), 3.77 (s, 2H), 2.90-2.78 (m, 2H), 2.71-2.66 (m, 2H), 1.88-1.62 (m, 6H).

### Example 118: N-(1-(4-(2-(2-Aminopyridin-3-yl)-5-phenyl-3H-imidazo[4,5-b]pyridin-3-yl)benzyl)azepan-3-yl)-4-formyl-3-hydroxybenzamide

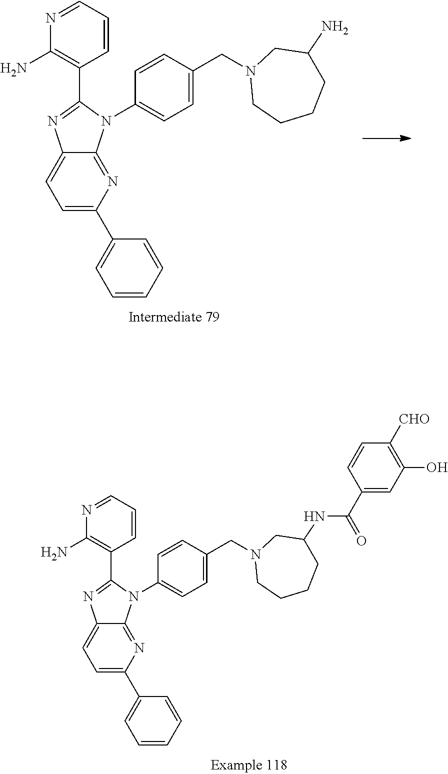 [SMILES: Nc1ncccc1-c1nc2ccc(-c3ccccc3)nc2n1-c1ccc(CN2CCCCC(N)C2)cc1 ; Nc1ncccc1-c1nc2ccc(-c3ccccc3)nc2n1-c1ccc(CN2CCCCC(NC(=O)c3ccc(C=O)c(O)c3)C2)cc1]

Following the general procedures described in Example 36, the reaction was carried out using 4-formyl-3-hydroxybenzoic acid and Intermediate 79. After purified by prep-HPLC (column: Phenomenex C₁₈ 150×25 mm×10 m; mobile phase: [water (NH₄HCO₃)-ACN]; gradient: 44%-74% B over 14 min), N-(1-(4-(2-(2-aminopyridin-3-yl)-5-phenyl-3H-imidazo[4,5-b]pyridin-3-yl)benzyl)azepan-3-yl)-4-formyl-3-hydroxybenzamide (Example 118, 11.6 mg, yield: 18%) was obtained as a yellow solid. MS: m/z=638.4 [M+H]r. ¹H NMR (400 MHz, Dimethylsulfoxide-d₆) δ 11.07-10.65 (m, 1H), 10.26 (s, 1H), 8.26-8.24 (m, 1H), 8.03-7.95 (m, 3H), 7.93 (dd, J=4.4, 1.2 Hz, 1H), 7.63 (d, J=8.0 Hz, 1H), 7.57-7.52 (m, 2H), 7.46-7.36 (m, 7H), 7.31 (d, J=8.0 Hz, 1H), 7.10-7.04 (m, 3H), 6.31 (dd, J=7.6, 4.8 Hz, 1H), 4.16-4.05 (m, 1H), 3.79 (s, 2H), 2.88-2.68 (m, 4H), 1.78-1.54 (m, 6H).

### Example 119: 5-(4-(4-(2-(2-Aminopyridin-3-yl)-5-phenyl-3H-imidazo[4,5-b]pyridin-3-yl)benzyl)-1,4-diazepane-1-carbonyl)-2-hydroxybenzaldehyde

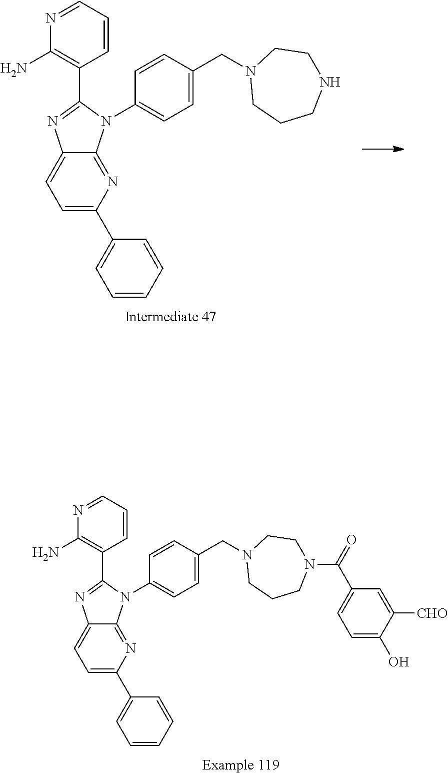 [SMILES: Nc1ncccc1-c1nc2ccc(-c3ccccc3)nc2n1-c1ccc(CN2CCCNCC2)cc1 ; Nc1ncccc1-c1nc2ccc(-c3ccccc3)nc2n1-c1ccc(CN2CCCN(C(=O)c3ccc(O)c(C=O)c3)CC2)cc1]

Following the general procedures described in Example 36, the reaction was carried out using 3-formyl-4-hydroxybenzoic acid and Intermediate 47. After purified by prep-HPLC (column: Phenomenex C18 150×25 mm×10 μm; mobile phase: [water (NH₃H₂O)-ACN]; gradient: 30%-60%, over 14 min), 5-(4-(4-(2-(2-aminopyridin-3-yl)-5-phenyl-3H-imidazo[4,5-b]pyridin-3-yl)benzyl)-1,4-diazepane-1-carbonyl)-2-hydroxybenzaldehyde (Example 119, 10.1 mg, yield: 7.4%) was obtained as a light-yellow solid. MS: m/z=624.4 [M+H]⁺. ¹H NMR (400 MHz, Dimethylsulfoxide-d₆) δ 10.26 (s, 1H), 8.27 (d, J=8.4 Hz, 1H), 8.00 (dd, J=14.8, 7.2 Hz, 4H), 7.56 (dd, J=8.4, 2.0 Hz, 1H), 7.50-7.37 (m, 9H), 7.16-7.16 (m, 1H), 7.07-7.02 (s, 2H), 7.01 (d, J=8.8 Hz, 1H), 6.38-6.27 (m, 1H), 3.73 (s, 2H), 3.65-3.57 (m, 2H), 3.50-3.41 (m, 2H), 2.71-2.59 (m, 4H), 1.86-1.76 (m, 2H).

### Example 120: 4-(4-(4-(2-(2-Aminopyridin-3-yl)-5-phenyl-3H-imidazo[4,5-b]pyridin-3-yl)benzyl)-1,4-diazepan-1-yl)-2-hydroxybenzaldehyde

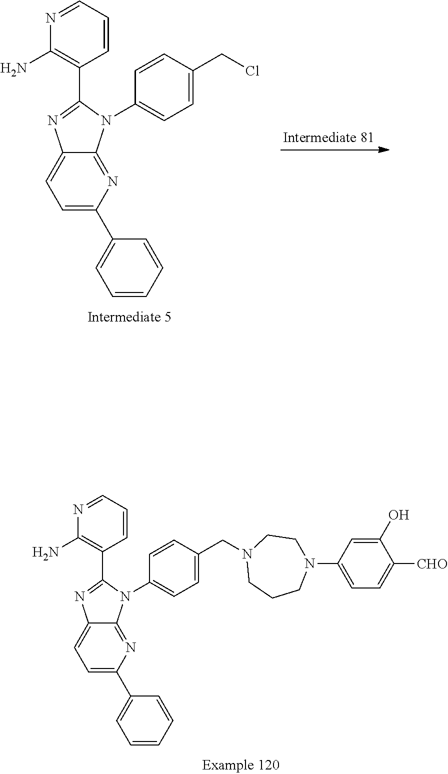 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CCCN(c3ccc(C=O)c(O)c3)CC2)cc1]

Following the general procedures described in Example 73, the reaction was carried out using Intermediate 5 and Intermediate 81. After purified by prep-HPLC (column: Waters xbridge 150×25 mm×10 μm; mobile phase: [water (NH₄HCO₃)-ACN]; gradient: 40%-80% B over 18 min), 4-(4-(4-(2-(2-aminopyridin-3-yl)-5-phenyl-3H-imidazo[4,5-b]pyridin-3-yl)benzyl)-1,4-diazepan-1-yl)-2-hydroxybenzaldehyde (Example 120, 24.8 mg, yield: 11%) was obtained as a light yellow solid. MS: m/z=596.4 [M+H]⁺. ¹H NMR (400 MHz, Dimethylsulfoxide-d₆) δ 11.25 (s, 1H), 9.63 (s, 1H), 8.27 (d, J=8.4 Hz, 1H), 8.04-7.97 (m, 4H), 7.49-7.43 (m, 7H), 7.42-7.39 (m, 1H), 7.12 (dd, J=7.6, 1.6 Hz, 1H), 7.05 (s, 2H), 6.45 (dd, J=8.8, 2.0 Hz, 1H), 6.34 (dd, J=7.6, 4.8 Hz, 1H), 6.14 (d, J=2.0 Hz, 1H), 3.74 (s, 2H), 3.65-3.62 (m, 2H), 3.60-3.57 (m, 2H), 2.75-2.73 (m, 2H), 2.58-2.56 (m, 2H), 1.90-1.89 (m, 2H).

### Example 121: 5-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde

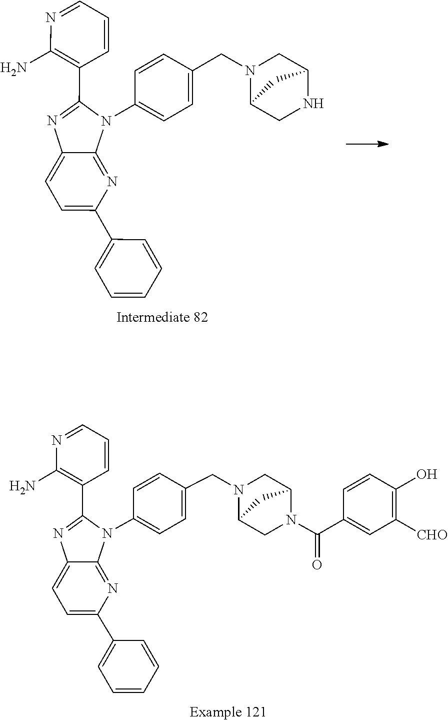 [SMILES: Nc1ncccc1-c1nc2ccc(-c3ccccc3)nc2n1-c1ccc(CN2C[C@@H]3C[C@H]2CN3)cc1 ; Nc1ncccc1-c1nc2ccc(-c3ccccc3)nc2n1-c1ccc(CN2C[C@@H]3C[C@H]2CN3C(=O)c2ccc(O)c(C=O)c2)cc1]

Following the general procedures described in Example 36, the reaction was carried out using 3-formyl-4-hydroxybenzoic acid and Intermediate 82. After purified by prep-HPLC (column: Phenomenex C18 150×25 mm×10 μm; mobile phase: [water(NH₄HCO₃)-ACN]; gradient: 28%-58% B over 14 min), 5-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde (Intermediate 121, 14.1 mg, yield: 10%) was obtained as a yellow powder. MS: m/z=622.2 [M+H]⁺. ¹H NMR (400 MHz, Dimethysulfoxide-d₆) δ 10.27 (s, 1H), 8.27 (d, J=8.4 Hz, 1H), 8.05-7.96 (m, 4H), 7.81-7.56 (m, 2H), 7.52-7.37 (m, 7H), 7.20-7.13 (m, 1H), 7.06-6.95 (m, 3H), 6.45-6.34 (m, 1H), 4.70-4.18 (m, 1H), 3.91-3.75 (m, 2H), 3.66-3.49 (m, 3H), 2.93-2.85 (m, 1H), 2.76-2.69 (m, 1H), 1.93-1.68 (m, 2H).

### Example 122: 4-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde

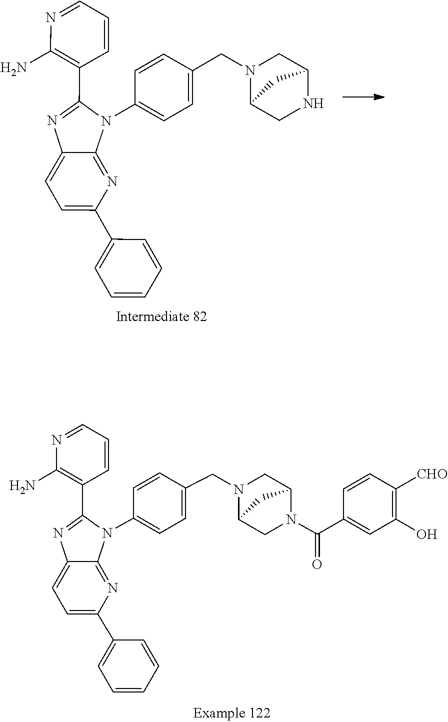 [SMILES: Nc1ncccc1-c1nc2ccc(-c3ccccc3)nc2n1-c1ccc(CN2C[C@@H]3C[C@H]2CN3)cc1 ; Nc1ncccc1-c1nc2ccc(-c3ccccc3)nc2n1-c1ccc(CN2C[C@@H]3C[C@H]2CN3C(=O)c2ccc(C=O)c(O)c2)cc1]

Following the general procedures described in Example 36, the reaction was carried out using 4-formyl-3-hydroxybenzoic acid and Intermediate 82. After purified by prep-HPLC (column: Phenomenex C18 150×25 mm×10 μm; mobile phase: [water(NH₄HCO₃)-ACN]; gradient: 30%-60% B over 10 min) and purified by prep-TLC (CH₂Cl₂:MeOH=10:1), 4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde (Example 122, 19.8 mg, yield: 13%) was obtained as a yellow solid. MS: m/z=622.4 [M+H]⁺. ¹H NMR (400 MHz, Methanol-d₄) δ 10.17-10.10 (m, 1H), 8.22-8.16 (m, 1H), 8.06-8.01 (m, 2H), 8.00-7.96 (m, 1H), 7.96-7.92 (m, 1H), 7.81 (dd, J=8.0, 1.6 Hz, 1H), 7.60 (d, J=8.4 Hz, 1H), 7.55 (d, J=8.4 Hz, 1H), 7.48-7.40 (m, 4H), 7.39-7.31 (m, 2H), 7.18-7.03 (m, 2H), 6.52-6.44 (m, 1H), 3.95-3.84 (m, 2H), 3.81-3.62 (m, 2H), 3.55-3.43 (m, 2H), 2.99-2.84 (m, 2H), 2.12-1.98 (m, 1H), 1.94-1.84 (m, 1H).

### Example 123: 4-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde

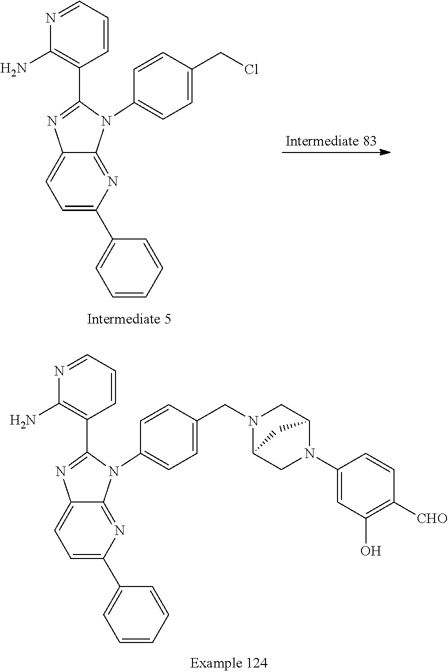 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2C[C@@H]3C[C@H]2CN3c2ccc(C=O)c(O)c2)cc1]

Following the general procedures described in Example 73, the reaction was carried out using Intermediate 5 and Intermediate 83. After purified by prep-HPLC (column: Waters xbridge 150×25 mm×10 μm; mobile phase: [water(NH₄HCO₃)-ACN]; gradient: 51%-81% B over 14 min), 4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde (Example 123, 32.7 mg, yield, 15% for two steps) was obtained as a yellow powder. MS: m/z=594.3 [M+H]⁺, ¹H NMR (400 MHz, Methanol-d₄) δ 9.48 (s, 1H), 8.19 (d, J=8.4 Hz, 1H), 8.03 (d, J=7.6 Hz, 2H), 7.99-7.96 (m, 1H), 7.93 (d, J=8.4 Hz, 1H), 7.55 (d, J=8.4 Hz, 2H), 7.45-7.36 (m, 6H), 7.33-7.30 (m, 1H), 6.47 (dd, J=7.6, 5.2 Hz, 1H), 6.35-6.28 (m, 1H), 6.02 (s, 1H), 4.53-4.52 (m, 1H), 3.85 (s, 2H), 3.72-3.71 (m, 1H), 3.57-3.53 (m, 1H), 3.47-3.43 (m, 1H), 3.03-2.99 (m, 1H), 2.76-2.70 (m, 1H), 2.13-2.08 (m, 1H), 1.95-1.89 (m, 1H).

### Example 124: 4-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde

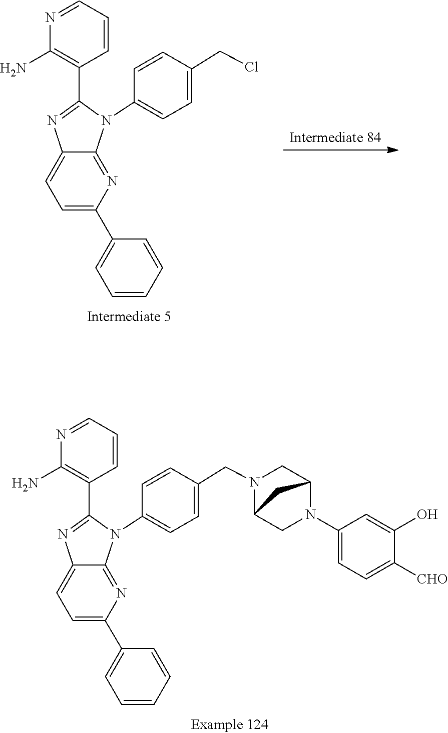 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2C[C@H]3C[C@@H]2CN3c2ccc(C=O)c(O)c2)cc1]

Following the general procedures described in Example 73, the reaction was carried out using Intermediate 5 and Intermediate 84. After purified by silica gel flash chromatography (Eluent of 0%-10% MeOH in CH₂Cl₂), 4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde (Example 124, 120 mg, yield: 16% for two steps) was obtained as a light yellow solid. MS: m/z=594.4 [M+H]⁺. ¹H NMR (400 MHz, Dimethylsulfoxide-d₆) δ 11.38 (br s, 1H), 9.61 (s, 1H), 8.25 (d, J=8.4 Hz, 1H), 8.05-7.95 (m, 4H), 7.50-7.35 (m, 8H), 7.17-7.12 (m, 1H), 7.01 (br s, 2H), 6.38 (dd, J=7.6, 4.8 Hz, 1H), 6.38-6.30 (m, 1H), 6.10-5.98 (m, 1H), 4.55-4.53 (m, 1H), 3.84-3.76 (m, 2H), 3.63-3.62 (m, 1H), 3.45-3.40 (m, 2H), 3.19-3.16 (m, 1H), 2.95-2.88 (m, 1H), 1.99-1.94 (m, 1H), 1.85-1.78 (m, 1H).

### Example 125: 4-(5-(4-(2-(2-aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde

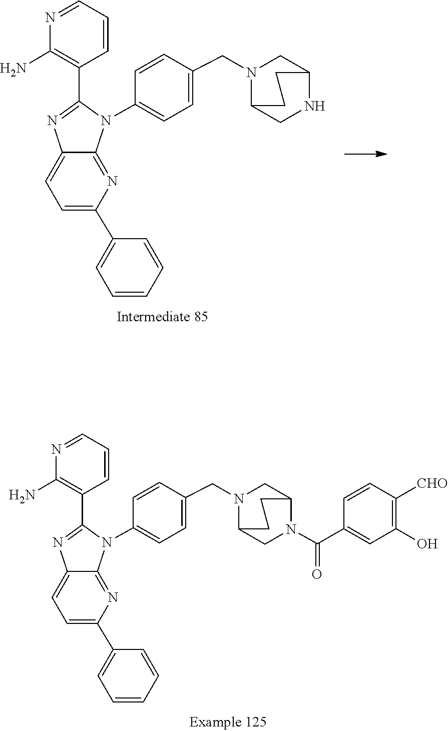 [SMILES: Nc1ncccc1-c1nc2ccc(-c3ccccc3)nc2n1-c1ccc(CN2CC3CCC2CN3)cc1 ; Nc1ncccc1-c1nc2ccc(-c3ccccc3)nc2n1-c1ccc(CN2CC3CCC2CN3C(=O)c2ccc(C=O)c(O)c2)cc1]

Following the general procedures described in Example 36, the reaction was carried out using 4-formyl-3-hydroxybenzoic acid and Intermediate 85. After purified by prep-HPLC (column: Waters xbridge 150×25 mm×10 μm; mobile phase: [water(NH₄HCO₃)-ACN]; gradient: 41%-71% B over 53 min), 4-(5-(4-(2-(2-aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde (Example 125, 22.3 mg, yield: 17%) was obtained as a yellow powder. MS: m/z=636.3 [M+H]⁺. ¹H NMR (400 MHz, Dimethysulfoxide-d₆) δ 11.18-10.75 (m, 1H), 10.33-10.23 (m, 1H), 8.27 (d, J=8.4 Hz, 1H), 8.04-7.97 (m, 4H), 7.73-7.66 (m, 1H), 7.55-7.50 (m, 2H), 7.49-7.43 (m, 4H), 7.42-7.38 (m, 1H), 7.19-7.13 (m, 1H), 7.06-6.91 (m, 4H), 6.41-6.36 (m, 1H), 3.87-3.82 (m, 2H), 3.59-3.45 (m, 2H), 3.11-2.87 (m, 2H), 2.85-2.62 (m, 2H), 2.11-1.70 (m, 4H).

### Example 126: 5-(5-(4-(2-(2-Aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde

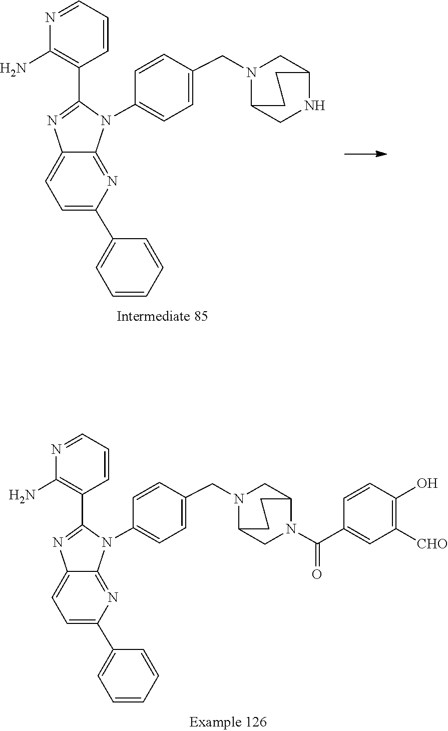 [SMILES: Nc1ncccc1-c1nc2ccc(-c3ccccc3)nc2n1-c1ccc(CN2CC3CCC2CN3)cc1 ; Nc1ncccc1-c1nc2ccc(-c3ccccc3)nc2n1-c1ccc(CN2CC3CCC2CN3C(=O)c2ccc(O)c(C=O)c2)cc1]

Following the general procedures described in Example 36, the reaction was carried out using 3-formyl-4-hydroxybenzoic acid and Intermediate 85. After purified by prep-HPLC (column: Phenomenex C18 150×25 mm×10 μm; mobile phase: [water(NH₄HCO₃)-ACN]; gradient: 30%-60% B over 10 min), 5-(5-(4-(2-(2-aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde (Example 126, 18.5 mg, yield: 14%) was obtained as a yellow powder. MS: m/z=636.4 [M+H]⁺. ¹H NMR (400 MHz, Dimethysulfoxide-d₆) δ 11.69-10.71 (m, 1H), 10.27 (s, 1H), 8.27 (d, J=8.4 Hz, 1H), 8.06-7.96 (m, 4H), 7.77-7.62 (m, 1H), 7.57-7.37 (m, 8H), 7.15 (d, J=6.8 Hz, 1H), 7.06-6.94 (m, 3H), 6.42-6.33 (m, 1H), 3.86-3.70 (m, 3H), 3.58-3.41 (m, 1H), 3.09-2.88 (m, 2H), 2.87-2.64 (m, 2H), 2.11-1.87 (m, 2H), 1.81-1.59 (m, 2H).

### Example 127: 4-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-2-hydroxybenzaldehyde

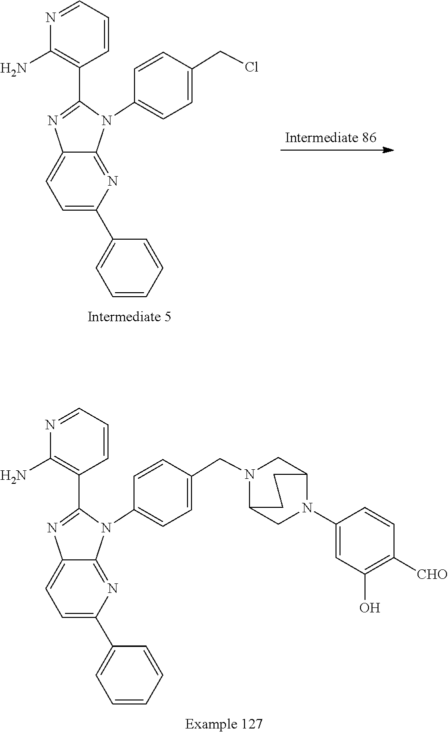 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2CC3CCC2CN3c2ccc(C=O)c(O)c2)cc1]

Following the general procedures described in Example 73, the reaction was carried out using Intermediate 5 and Intermediate 86. After purified by prep-HPLC (column: Waters xbridge 150×25 mm×10 μm; mobile phase: [water(NH₄HCO₃)-ACN]; gradient: 55%-85% B over 14 min), 4-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-2-hydroxybenzaldehyde (Example 127, 23.2 mg, yield, 10% for two steps) was obtained as a yellow powder. MS: m/z=608.3 [M+H]⁺, ¹H NMR (400 MHz, Methanol-d₄) δ 9.48 (s, 1H), 8.19 (d, J=8.4 Hz, 1H), 8.05-8.01 (m, 2H), 7.98 (dd, J=4.8, 1.6 Hz, 1H), 7.94 (d, J=8.4 Hz, 1H), 7.59 (d, J=8.4 Hz, 2H), 7.49-7.28 (m, 8H), 6.48 (dd, J=7.6, 4.8 Hz, 1H), 6.44-6.35 (m, 1H), 6.11-6.04 (m, 1H), 4.16-4.09 (m, 1H), 3.94-3.85 (m, 3H), 3.38-3.35 (m, 1H), 3.10-3.00 (m, 3H), 2.26-2.17 (m, 1H), 2.00-1.93 (m, 2H), 1.75-1.67 (m, 1H).

### Example 128: 2-((1-(4-(2-(2-Aminopyridin-3-yl)-5-phenyl-3H-imidazo[4,5-b]pyridin-3-yl)benzyl)piperidin-4-yl)oxy)-6-hydroxybenzaldehyde

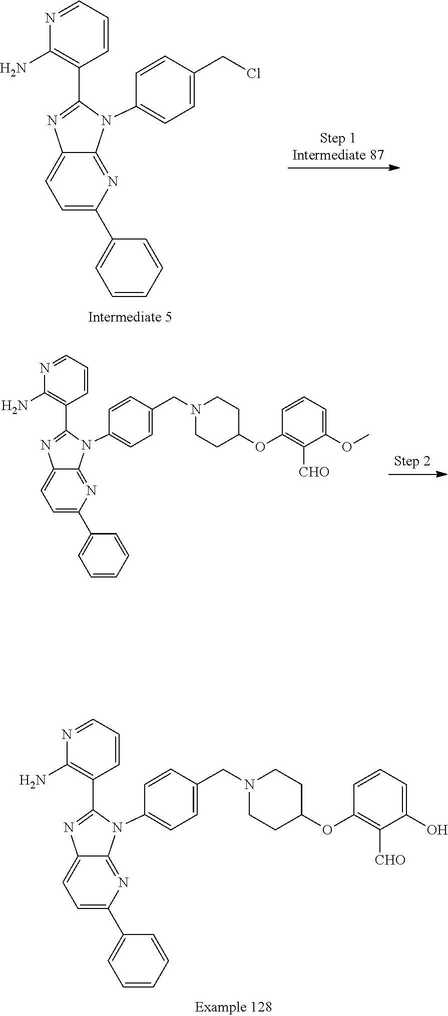 [SMILES: Nc1ncccc1-c1nc2ccc(-c3ccccc3)nc2n1-c1ccc(CCl)cc1 ; COc1cccc(OC2CCN(Cc3ccc(-n4c(-c5cccnc5N)nc5ccc(-c6ccccc6)nc54)cc3)CC2)c1C=O ; Nc1ncccc1-c1nc2ccc(-c3ccccc3)nc2n1-c1ccc(CN2CCC(Oc3cccc(O)c3C=O)CC2)cc1]

### Step 1: 2-((1-(4-(2-(2-Aminopyridin-3-yl)-5-phenyl-3H-imidazo[4,5-b]pyridin-3-yl)benzyl)piperidin-4-yl)oxy)-6-methoxybenzaldehyde

To a solution of Intermediate 5 (250 mg, 606 μmol) in DMF (2 mL) were added Intermediate 87 (157 mg, 578 μmol, HCl salt), NaI (27.2 mg, 182 μmol) and K₂CO₃ (419 mg, 3.03 mmol). The mixture was stirred at 80 °C for 2 hr under N₂. The reaction mixture was diluted with H₂O (10 mL), filtered and concentrated under reduced pressure. After purified by silica gel flash chromatography (Eluent of 0%-5% MeOH in CH₂Cl₂), 2-((1-(4-(2-(2-aminopyridin-3-yl)-5-phenyl-3H-imidazo[4,5-b]pyridin-3-yl)benzyl)piperidin-4-yl)oxy)-6-methoxybenzaldehyde (Example 128, 250 mg, yield: 56%) was obtained as a yellow solid. MS: m/z=611.4 [M+H]⁺. ¹H NMR (400 MHz, Dimethylsulfoxide-d₆) δ 10.41 (s, 1H), 8.27 (d, J=8.4 Hz, 1H), 8.04-7.95 (m, 4H), 7.54-7.43 (m, 7H), 7.40 (d, J=7.2 Hz, 1H), 7.15 (d, J=7.2 Hz, 1H), 7.02 (br s, 2H), 6.81 (d, J=8.4 Hz, 1H), 6.71 (d, J=8.4 Hz, 1H), 6.37 (dd, J=7.2, 4.8 Hz, 1H), 4.60-4.50 (m, 1H), 3.80 (s, 3H), 3.59 (s, 2H), 2.69-2.61 (m, 2H), 2.40-2.30 (m, 2H), 1.99-1.91 (m, 2H), 1.80-1.65 (m, 2H).

### Step 2: 2-((1-(4-(2-(2-Aminopyridin-3-yl)-5-phenyl-3H-imidazo[4,5-b]pyridin-3-yl)benzyl)piperidin-4-yl)oxy)-6-hydroxybenzaldehyde

To a mixture of 2-((1-(4-(2-(2-aminopyridin-3-yl)-5-phenyl-3H-imidazo[4,5-b]pyridin-3-yl)benzyl)piperidin-4-yl)oxy)-6-methoxybenzaldehyde (100 mg, 163 μmol) in CH₂Cl₂ (15 mL) was added BBr₃ (820 mg, 3.27 mmol) at 0 °C The mixture was stirred at 20 °C for 16 hr. The reaction mixture was quenched with NaHCO₃ (30 mL), diluted with H₂O (20 mL), and extracted with CH₂Cl₂ (30 mL×3). The combined organic layers were dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 62%-92% B over 12 min), 2-((1-(4-(2-(2-aminopyridin-3-yl)-5-phenyl-3H-imidazo[4,5-b]pyridin-3-yl)benzyl)piperidin-4-yl)oxy)-6-hydroxybenzaldehyde (Example 128, 12.4 mg, yield: 12%) was obtained as a white powder. MS: m/z=597.4 [M+H]⁺. ¹H NMR (400 MHz, Dimethylsulfoxide-d_{f};) δ 11.74 (br s, 1H), 10.33 (s, 1H), 8.27 (d, J=8.4 Hz, 1H), 8.05-7.96 (m, 4H), 7.52-7.43 (m, 7H), 7.41-7.36 (m, 1H), 7.15 (d, J=7.2 Hz, 1H), 7.03 (br s, 2H), 6.68 (d, J=8.4 Hz, 1H), 6.49 (d, J=8.4 Hz, 1H), 6.37 (dd, J=7.6, 4.8 Hz, 1H), 4.70-4.58 (m, 1H), 3.60 (s, 2H), 2.70-2.60 (m, 2H), 2.40-2.30 (m, 2H), 2.30-1.93 (m, 2H), 1.83-1.6 (m, 2H).

### Example 129: 4-((1-(4-(2-(2-Aminopyridin-3-yl)-5-phenyl-3H-imidazo[4,5-b]pyridin-3-yl)benzyl)piperidin-4-yl)oxy)-2-hydroxybenzaldehyde

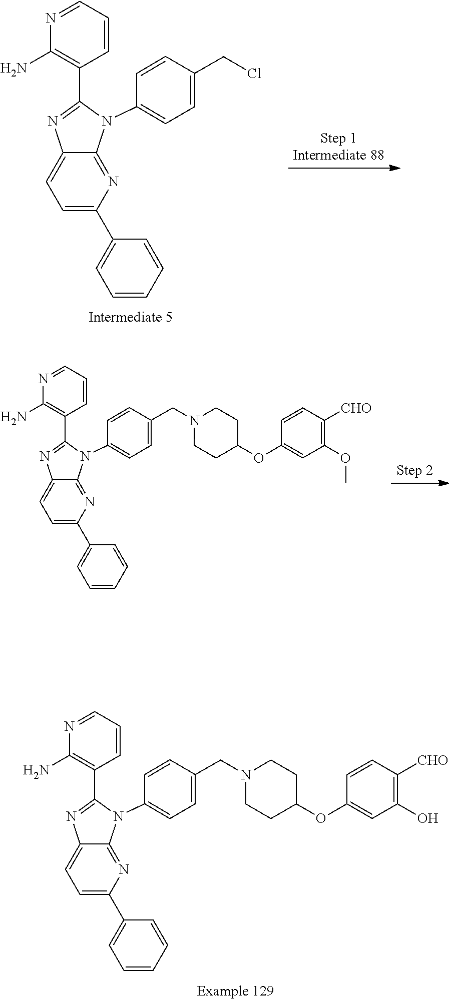 [SMILES: Nc1ncccc1-c1nc2ccc(-c3ccccc3)nc2n1-c1ccc(CCl)cc1 ; COc1cc(OC2CCN(Cc3ccc(-n4c(-c5cccnc5N)nc5ccc(-c6ccccc6)nc54)cc3)CC2)ccc1C=O ; Nc1ncccc1-c1nc2ccc(-c3ccccc3)nc2n1-c1ccc(CN2CCC(Oc3ccc(C=O)c(O)c3)CC2)cc1]

Following the general procedures described in Example 128, the reactions were carried out using Intermediate 5 and Intermediate 88. After purified by prep-HPLC (column: Waters xbridge 150×25 mm×10 μm; mobile phase: [water (NH₄HCO₃)-ACN]; gradient: 52%-72% B over 14 min), 4-((1-(4-(2-(2-aminopyridin-3-yl)-5-phenyl-3H-imidazo[4,5-b]pyridin-3-yl)benzyl)piperidin-4-yl)oxy)-2-hydroxybenzaldehyde (Example 129, 5.7 mg, yield: 5.6%) was obtained as a light yellow solid. MS: m/z=597.3 [M+H]⁺. ¹H NMR (400 MHz, Dimethylsulfoxide-d₆) δ 11.03-10.89 (m, 1H), 9.99 (s, 1H), 8.27 (d, J=8.4 Hz, 1H), 8.04-7.97 (m, 4H), 7.61 (d, J=8.8 Hz, 1H), 7.50-7.44 (m, 6H), 7.43-7.38 (m, 1H), 7.18-7.14 (m, 1H), 7.02 (s, 2H), 6.59 (dd, J=8.8, 2.0 Hz, 1H), 6.50 (d, J=2.0 Hz, 1H), 6.38 (dd, J=7.6, 4.8 Hz, 1H), 4.58-4.49 (m, 1H), 3.61 (s, 2H), 2.75-2.70 (m, 2H), 2.39-2.33 (m, 2H), 2.03-1.97 (m, 2H), 1.74-1.66 (m, 2H).

### Example 130: 5-((1-(4-(2-(2-Aminopyridin-3-yl)-5-phenyl-3H-imidazo[4,5-b]pyridin-3-yl)benzyl)piperidin-4-yl)oxy)-2-hydroxybenzaldehyde

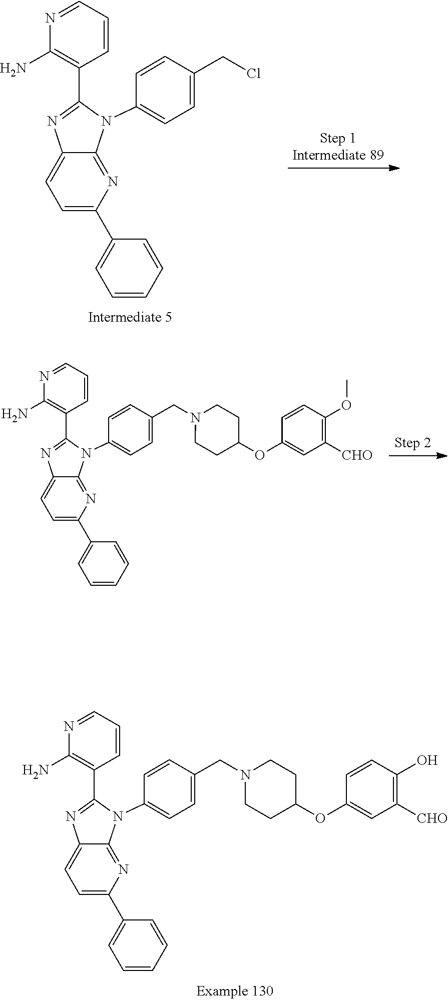 [SMILES: Nc1ncccc1-c1nc2ccc(-c3ccccc3)nc2n1-c1ccc(CCl)cc1 ; COc1ccc(OC2CCN(Cc3ccc(-n4c(-c5cccnc5N)nc5ccc(-c6ccccc6)nc54)cc3)CC2)cc1C=O ; Nc1ncccc1-c1nc2ccc(-c3ccccc3)nc2n1-c1ccc(CN2CCC(Oc3ccc(O)c(C=O)c3)CC2)cc1]

Following the general procedures described in Example 128, the reactions were carried out using Intermediate 5 and Intermediate 89. After purified by prep-HPLC (column: Waters xbridge 150×25 mm×10 μm; mobile phase: [water (NH₄HCO₃)-ACN]; gradient: 50%-80% B over 10 min), 5-((1-(4-(2-(2-aminopyridin-3-yl)-5-phenyl-3H-imidazo[4,5-b]pyridin-3-yl)benzyl)piperidin-4-yl)oxy)-2-hydroxybenzaldehyde (Example 130, 8.6 mg, yield: 5.9% for two steps) was obtained as a light yellow solid. MS: m/z=int[M+H]⁺. ¹H NMR (400 MHz, Dimethylsulfoxide-d₆) δ 10.22 (s, 1H), 8.27 (d, J=8.4 Hz, 1H), 8.04-7.98 (m, 4H), 7.50-7.44 (m, 7H), 7.40 (d, J=7.2 Hz, 1H), 7.19-7.15 (m, 3H), 7.02 (s, 2H), 6.95-6.92 (m, 1H), 6.38 (dd, J=7.6, 4.8 Hz, 1H), 4.34-4.27 (m, 1H), 3.60 (s, 2H), 2.74-2.70 (m, 2H), 2.32-2.26 (m, 2H), 1.97-1.90 (m, 2H), 1.68-1.62 (m, 2H).

### Example 131: 5-((3aR,6aS)-5-(4-(2-(2-Aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde

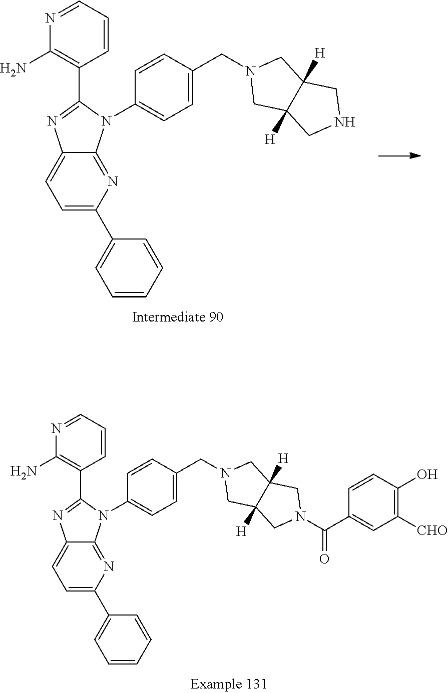 [SMILES: Nc1ncccc1-c1nc2ccc(-c3ccccc3)nc2n1-c1ccc(CN2C[C@H]3CNC[C@H]3C2)cc1 ; Nc1ncccc1-c1nc2ccc(-c3ccccc3)nc2n1-c1ccc(CN2C[C@@H]3CN(C(=O)c4ccc(O)c(C=O)c4)C[C@@H]3C2)cc1]

Following the general procedures described in Example 44, the reaction was carried out using 3-formyl-4-hydroxybenzoic acid and Intermediate 90. After purified by prep-HPLC (column: Boston Prime C18 150×30 mm×5 μm; mobile phase: [water (NH₃H₂O+NH₄HCO₃)-ACN]; gradient: 45%-75% B over 7 min), 5-((3aR,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde (Example 131, 9.1 mg, yield: 8%) was obtained as a yellow solid. MS: m/z=636.3 [M+H]⁺. ¹H NMR (400 MHz, Dimethylsulfoxide-d₆) δ 10.25 (s, 1H), 8.27 (d, J=8.8 Hz, 1H), 8.04-7.97 (m, 4H), 7.80 (d, J=2.0 Hz, 1H), 7.68 (dd, J=8.4, 2.4 Hz, 1H), 7.49-7.36 (m, 9H), 7.14 (dd, J=7.6, 1.2 Hz, 1H), 7.03-7.01 (m, 2H), 6.36 (dd, J=7.6, 2.0 Hz, 1H), 3.76-3.60 (m, 4H), 2.80 (s, 2H), 2.57-2.53 (m, 4H), 2.45-2.35 (m, 2H).

### Example 132: 4-((3aR,6aS)-5-(4-(2-(2-Aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde

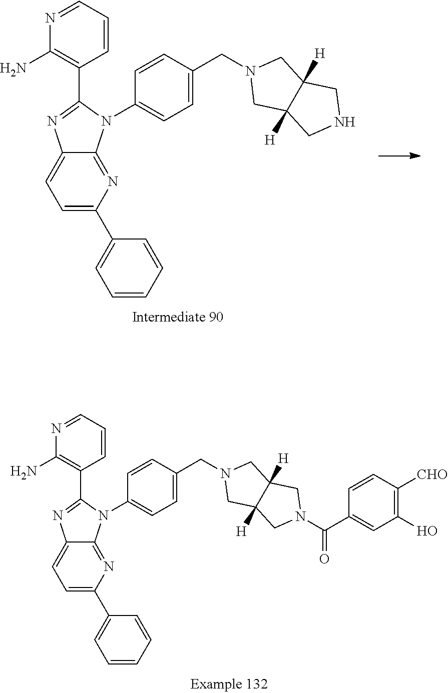 [SMILES: Nc1ncccc1-c1nc2ccc(-c3ccccc3)nc2n1-c1ccc(CN2C[C@H]3CNC[C@H]3C2)cc1 ; Nc1ncccc1-c1nc2ccc(-c3ccccc3)nc2n1-c1ccc(CN2C[C@@H]3CN(C(=O)c4ccc(C=O)c(O)c4)C[C@@H]3C2)cc1]

Following the general procedures described in Example 44, the reaction was carried out using 4-formyl-3-hydroxybenzoic acid and Intermediate 90. After purified by prep-TLC (CH₂Cl₂:MeOH=10:1) and prep-HPLC (column: Boston Prime C18 150×30 mm×5 μm; mobile phase: [water (NH₃H₂O+NH₄HCO₃)-ACN]; gradient: 50%-80% B over 7 min), 4-((3aR,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde (Example 132, 12.6 mg, yield: 10%) was obtained as an off-white solid. MS: m/z=658.2 [M+Na]⁺. ¹H NMR (400 MHz, Dimethylsulfoxide-d₆)S 10.92 (s, 1H), 10.26 (s, 1H), 8.27 (d, J=8.4 Hz, 1H), 8.03-7.97 (m, 4H), 7.67 (d, J=8.0 Hz, 1H), 7.49-7.36 (m, 7H), 7.15 (d, J=7.6 Hz, 1H), 7.05-6.99 (m, 4H), 6.40-6.34 (m, 1H), 3.71-3.53 (m, 4H), 3.27-3.15 (m, 2H), 2.88-2.78 (m, 2H), 2.62-2.57 (m, 2H), 2.47-2.34 (m, 2H).

### Example 133: 4-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde

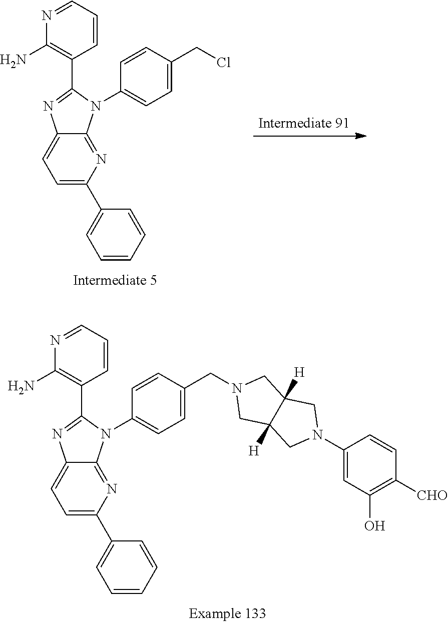 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2C[C@@H]3CN(c4ccc(C=O)c(O)c4)C[C@@H]3C2)cc1]

To a mixture of Intermediate 5 (200 mg, 485 μmol) and Intermediate 91 (143 mg, 534 μmol, HCl) in DMF (3 mL) was added DIEA (251 mg, 1.94 mmol). The mixture was stirred at 80 °C for 12 hr under N₂. The mixture was diluted with H₂O (50 mL) and extracted with EtOAc (50 mL×3). The combined organic layers were washed with brine (50 mL), dried over Na₂SO₄, filtered and concentrated. After purified by prep-HPLC (column: Phenomenex C18 80×40 mm×3 μm; mobile phase: [water (NH₄HCO₃)-ACN]; gradient: 53%-83% B over 9 min), 4-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde (Example 133, 61.3 mg, yield: 21% for two steps) was obtained as a yellow solid. MS: m/z=608.2 [M+H]⁺. 1H NMR (400 MHz, Dimethylsulfoxide-d₆) δ 11.27 (br s, 1H), 9.66 (s, 1H), 8.26 (d, J=8.4 Hz, 1H), 8.06-7.95 (m, 4H), 7.48-7.37 (m, 8H), 7.14 (dd, J=7.6, 1.6 Hz, 1H), 7.03 (br s, 2H), 6.37 (dd, J=7.6, 4.8 Hz, 1H), 6.28 (dd, J=8.8, 2.0 Hz, 1H), 5.98 (d, J=2.0 Hz, 1H), 3.68 (s, 2H), 3.60-3.56 (m, 2H), 3.27-3.21 (m, 2H), 3.01-2.92 (m, 2H), 2.68-2.62 (m, 2H), 2.58-2.53 (m, 2H).

### Example 134: 5-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde

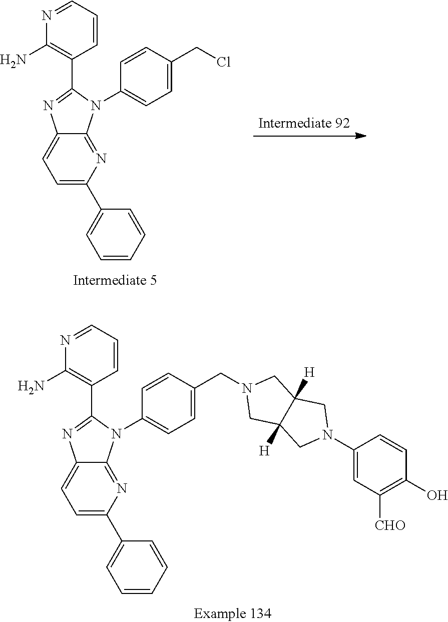 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(CN2C[C@@H]3CN(c4ccc(O)c(C=O)c4)C[C@@H]3C2)cc1]

Following the general procedures described in Example 133, the reaction was carried out using Intermediate 5 and Intermediate 92. After purified by silica gel flash chromatography (Eluent of 0%~10% MeOH in CH₂Cl₂), 5-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde (Example 134, 14.6 mg, yield: 14% for two steps) was obtained as a yellow solid. MS: m/z=608.3 [M+H]⁺. ¹H NMR (400 MHz, Dimethylsulfoxide-d₆) 10.22 (s, 1H), 9.97 (s, 1H), 8.26 (d, J=8.4 Hz, 1H), 8.04-8.01 (m, 2H), 8.00-7.97 (m, 2H), 7.49-7.37 (m, 7H), 7.14 (dd, J=7.6, 2.0 Hz, 1H), 7.05-6.99 (m, 3H), 6.92-6.87 (m, 2H), 6.36 (dd, J=7.6, 4.8 Hz, 1H), 3.66 (s, 2H), 3.31-3.27 (m, 2H), 3.06-2.95 (m, 2H), 2.92-2.85 (m, 2H), 2.75-2.69 (m, 2H), 2.45-2.38 (m, 2H).

### Example 135: 5-((3aS,6aS)-5-(4-(2-(2-Aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde

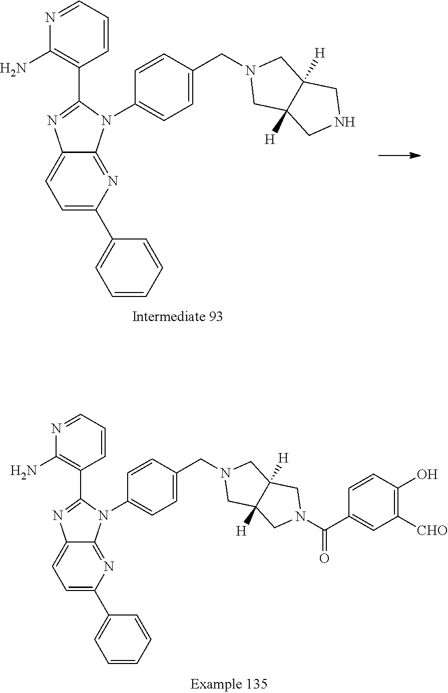 [SMILES: Nc1ncccc1-c1nc2ccc(-c3ccccc3)nc2n1-c1ccc(CN2C[C@H]3CNC[C@@H]3C2)cc1 ; Nc1ncccc1-c1nc2ccc(-c3ccccc3)nc2n1-c1ccc(CN2C[C@H]3CN(C(=O)c4ccc(O)c(C=O)c4)C[C@@H]3C2)cc1]

Following the general procedures described in Example 44, the reaction was carried out using 3-formyl-4-hydroxybenzoic acid and Intermediate 93. After purified by prep-HPLC (column: Boston Prime C18 150×30 mm×5 μm; mobile phase: [water (NH₃H₂O+NH₄HCO₃)-ACN]; gradient: 38%-68% B over 7 min), 5-((3aS,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde (Example 135, 32.7 mg, yield: 30%) was obtained as a yellow solid. MS: m/z=636.2 [M+H]⁺. ¹H NMR (400 MHz, Dimethylsulfoxide-d₆) δ 10.28 (s, 1H), 8.27 (d, J=8.4 Hz, 1H), 8.03-7.97 (m, 4H), 7.85 (d, J=2.4 Hz, 1H), 7.73 (dd, J=8.4, 2.4 Hz, 1H), 7.52-7.37 (m, 8H), 7.16 (dd, J=7.6, 1.6 Hz, 1H), 7.06-7.02 (m, 2H), 6.38 (dd, J=8.0, 4.8 Hz, 1H), 4.01-3.89 (m, 2H), 3.67-3.59 (m, 1H), 3.49-3.39 (m, 31H), 3.18-3.09 (m, 1H), 2.97-2.88 (m, 1H), 2.68-2.57 (m, 2H), 2.40-2.20 (m, 2H).

### Example 136:4-((3aS,6aS)-5-(4-(2-(2-Aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde

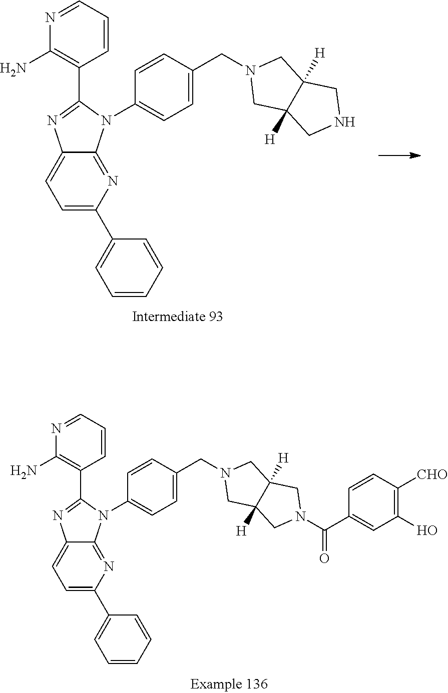 [SMILES: Nc1ncccc1-c1nc2ccc(-c3ccccc3)nc2n1-c1ccc(CN2C[C@H]3CNC[C@@H]3C2)cc1 ; Nc1ncccc1-c1nc2ccc(-c3ccccc3)nc2n1-c1ccc(CN2C[C@H]3CN(C(=O)c4ccc(C=O)c(O)c4)C[C@@H]3C2)cc1]

Following the general procedures described in Example 44, the reaction was carried out using 4-formyl-3-hydroxybenzoic acid and Intermediate 93. After purified by prep-HPLC (column: Welch Xtimate C18 150×25 mm×5 μm; mobile phase: [water (NH₃H₂O+NH₄HCO₃)-ACN]; gradient: 40%-70% B over 7 min), 4-((3aS,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde (Example 136, 9.2 mg, yield: 8%) was obtained as a yellow solid. MS: m/z=636.3 [M+H]⁺. ¹H NMR (400 MHz, Dimethylsulfoxide-d₆) δ 10.94 (s, 1H), 10.30 (s, 1H), 8.27 (d, J=8.0 Hz, 1H), 8.05-7.95 (m, 4H), 7.72-7.61 (m, 1H), 7.56-7.36 (m, 7H), 7.16 (dd, J=7.6, 1.6 Hz, 1H), 7.11-6.94 (m, 4H), 6.38 (dd, J=7.6, 4.4 Hz, 1H), 3.99 (br s, 2H), 3.73-3.65 (m, 1H), 3.33-3.21 (m, 4H), 3.18-3.10 (m, 1H), 3.01-2.73 (m, 2H), 2.65-2.52 (m, 2H).

### Example 137: 2-((4-(2-(2-Aminopyridin-3-yl)-5-phenyl-3H-imidazo[4,5-b]pyridin-3-yl)benzyl)oxy)-6-hydroxybenzaldehyde

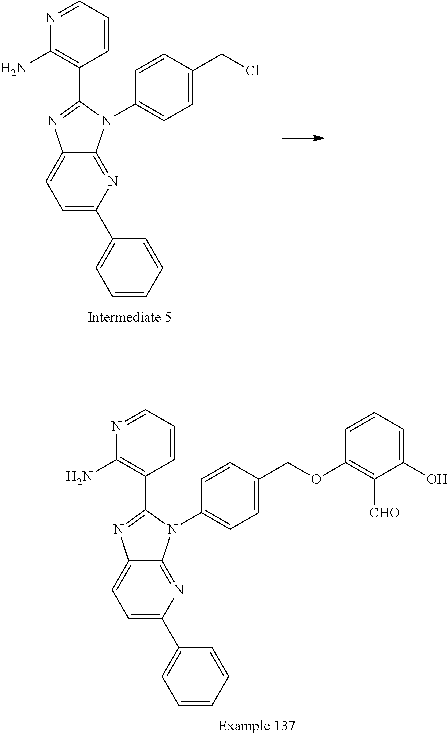 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(COc2cccc(O)c2C=O)cc1]

To a solution of Intermediate 5 (300 mg, 728 μmol), 2,6-dihydroxybenzaldehyde (101 mg, 728 μmol) in MeCN (5 mL) were added NaI (21.8 mg, 146 μmol) and Cs₂CO₃ (712 mg, 2.19 mmol). The mixture was stirred at 30 °C for 3 hr. The reaction mixture was filtered and concentrated under reduced pressure. After purified by prep-HPLC (column: Phenomenex Cis 150×25 mm×10 μm mobile phase: [water(NH₄HCO₃)-ACN]; gradient: 45%-75% B over 14 min) and prep-TLC (DCM:MeOH=10:1), 2-((4-(2-(2-aminopyridin-3-yl)-5-phenyl-3H-imidazo[4,5-b]pyridin-3-yl)benzyl)oxy)-6-hydroxybenzaldehyde (Example 137, 14 mg, yield: 4.3%) was obtained as a yellow solid. MS: m/z=514.3 [M+H]⁺. ¹H NMR (400 MHz, Chloroform-d) δ 11.99 (s, 1H), 10.48 (s, 1H), 8.15 (d, J=8.4 Hz, 1H), 8.06 (d, J=4.8 Hz, 1H), 8.01 (d, J=7.6 Hz, 2H), 7.82 (d, J=8.4 Hz, 1H), 7.62-7.57 (m, 2H), 7.54-7.49 (m, 2H), 7.47-7.41 (m, 3H), 7.40-7.35 (m, 1H), 7.82 (d, J=8.0 Hz, 1H), 6.75 (br s, 2H), 6.60 (d, J=8.4 Hz, 1H), 6.49 (d, J=8.4 Hz, 1H), 6.42-6.37 (m, 1H), 5.26 (s, 2H).

### Example 138: 4-((4-(2-(2-Aminopyridin-3-yl)-5-phenyl-3H-imidazo[4,5-b]pyridin-3-yl)benzyl)oxy)-2-hydroxybenzaldehyde

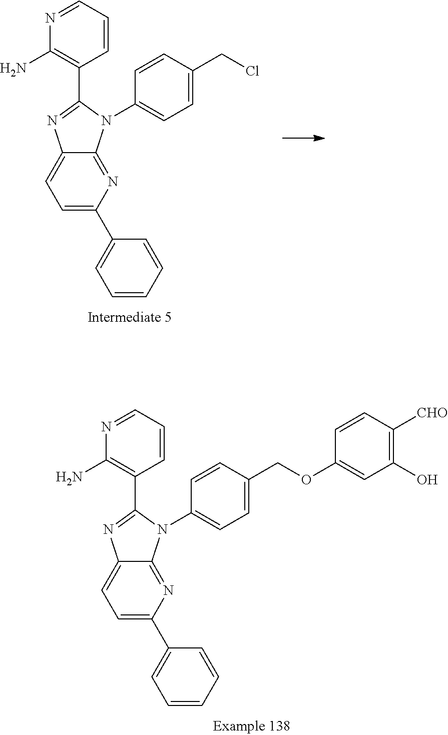 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(COc2ccc(C=O)c(O)c2)cc1]

To a solution of Intermediate 5 (100 mg, 243 μmol), 2,4-dihydroxybenzaldehyde (33.5 mg, 243 μmol) in MeCN (2 mL) were added NaI (7.28 mg, 45.6 μmol) and Cs₂CO₃ (237 mg, 728 μmol). The mixture was stirred at 45 °C for 16 hr. The reaction mixture was filtered and concentrated under reduced pressure. After purified by prep-HPLC (column: Phenomenex C₁₈ 150×25 mm×10 μm; mobile phase: [water(NH₄HCO₃)-ACN]; gradient: 45%-75% B over 10 min), 4-((4-(2-(2-aminopyridin-3-yl)-5-phenyl-3H-imidazo[4,5-b]pyridin-3-yl)benzyl)oxy)-2-hydroxybenzaldehyde (Example 138, 13 mg, yield: 10%) was obtained as a light yellow solid. MS: m/z=514.3 [M+H]⁺. ¹H NMR (400 MHz, Chlorofrom-d) δ 11.52 (s, 1H), 9.77 (s, 1H), 8.15 (d, J=8.4 Hz, 1H), 8.11-8.05 (m, 1H), 8.06-8.00 (m, 2H), 7.83 (d, J=8.4 Hz, 1H), 7.62-7.58 (m, 2H), 7.53-7.48 (m, 3H), 7.48-7.42 (m, 2H), 7.42-7.36 (m, 1H), 7.13 (dd, J=7.6, 1.2 Hz, 1H), 6.67 (dd, J=8.8, 2.4 Hz, 1H), 6.61 (br s, 2H), 6.56 (d, J=2.4 Hz, 1H), 6.43-6.38 (m, 1H), 5.24 (s, 2H).

### Example 139: 5-((4-(2-(2-Aminopyridin-3-yl)-5-phenyl-3H-imidazo[4,5-b]pyridin-3-yl)benzyl)oxy)-2-hydroxybenzaldehyde

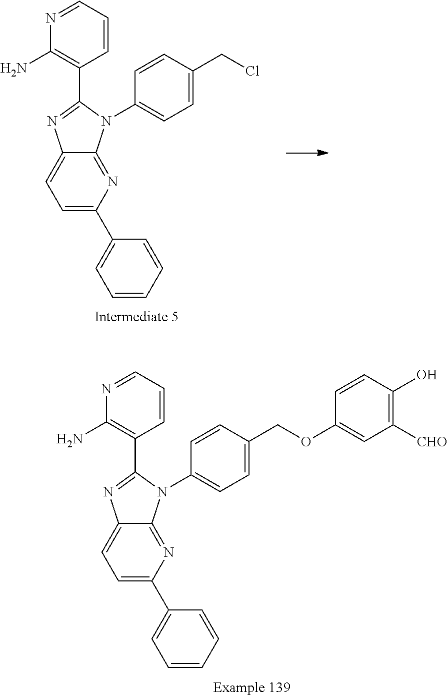 [SMILES: Nc1ncccc1-c1nc2ccc(-c3ccccc3)nc2n1-c1ccc(CCl)cc1 ; Nc1ncccc1-c1nc2ccc(-c3ccccc3)nc2n1-c1ccc(COc2ccc(O)c(C=O)c2)cc1]

To a solution of Intermediate 5 (500 mg, 1.21 mmol), 2,5-dihydroxybenzaldehyde (168 mg, 1.21 mmol) in DMF (4 mL) were added NaI (36.4 mg, 243 μmol) and Cs₂CO₃ (1.19 g, 3.64 mmol). The mixture was stirred at 80 °C for 2 hr. The reaction mixture was filtered and concentrated under reduced pressure. After purified by prep-HPLC (column: Phenomenex C₁₈ 150×25 mm×10 μm; mobile phase: [water(NH₄HCO₃)-ACN]; gradient: 33%-63% B over 14 min), 5-((4-(2-(2-aminopyridin-3-yl)-5-phenyl-3H-imidazo[4,5-b]pyridin-3-yl)benzyl)oxy)-2-hydroxybenzaldehyde (Example 139, 30.2 mg, yield: 4.8%) was obtained as a yellow lyophilized powder. MS: m/z=514.3 [M+H]⁺. ¹H NMR (400 MHz, Chlorofrom-d) δ 10.60-10.48 (m, 1H), 8.57-8.26 (m, 2H), 8.21 (d, J=8.4 Hz, 1H), 8.05-8.01 (m, 2H), 7.96-7.83 (m, 2H), 7.74-7.64 (m, 2H), 7.58-7.40 (m, 6H), 7.39-7.33 (m, 2H), 7.24-7.13 (m, 1H), 7.12-6.97 (m, 1H), 6.81-6.49 (m, 1H), 5.41-5.28 (m, 2H).

### Example 140: (S)—N-(1-(4-(2-(2-Aminopyridin-3-yl)-5-phenyl-3H-imidazo[4,5-b]pyridin-3-yl)phenyl)ethyl)-3-formyl-4-hydroxybenzamide

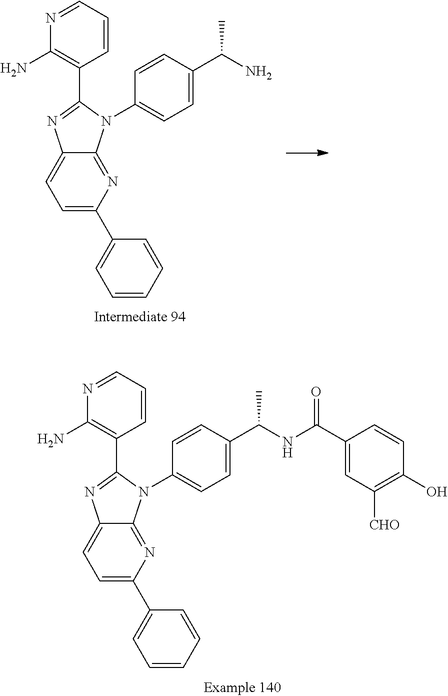 [SMILES: C[C@H](N)c1ccc(-n2c(-c3cccnc3N)nc3ccc(-c4ccccc4)nc32)cc1 ; C[C@H](NC(=O)c1ccc(O)c(C=O)c1)c1ccc(-n2c(-c3cccnc3N)nc3ccc(-c4ccccc4)nc32)cc1]

To a solution of Intermediate 94 (270 mg, 609 μmol) and DIEA (315 mg, 2.44 mmol) in CH₂Cl₂ (30 mL) was added 3-formyl-4-hydroxybenzoyl chloride (135 mg, 731 μmol) at 0 °C The mixture was stirred at 0 °C for 2 hr. The reaction mixture was diluted with NaHCO₃ (20 mL) and extracted with CH₂Cl₂ (50 mL×3). The combined organic layers were dried over Na₂SO₄, filtered and concentrated under reduced pressure. After purified by silica gel flash chromatography (Eluent of 0%-7% MeOH in CH₂Cl₂) and prep-HPLC (column: Waters xbridge 150×25 mm 10 μm; mobile phase: [water (NH₄HCO₃)-ACN]; gradient: 26%-56% B over 14 min), (S)—N-(1-(4-(2-(2-aminopyridin-3-yl)-5-phenyl-3H-imidazo[4,5-b]pyridin-3-yl)phenyl)ethyl)-3-formyl-4-hydroxybenzamide (Example 140, 4.1 mg, yield: 1.1%) was obtained as a light yellow powder. MS: m/z=inter [M+H]⁺. ¹H NMR (400 MHz, Dimethylsulfoxide-d₆) δ 10.32 (s, 1H), 8.92 (d, J=8.0 Hz, 1H), 8.30 (d, J=2.0 Hz, 1H), 8.26 (d, J=8.0 Hz, 1H), 8.06 (dd, J=8.8, 2.4 Hz, 1H), 8.03-7.96 (m, 4H), 7.55 (d, J=8.4 Hz, 2H), 7.49-7.44 (m, 4H), 7.42-7.35 (m, 1H), 7.18 (dd, J=7.6, 2.0 Hz, 1H), 7.04 (d, J=8.8 Hz, 1H), 6.95 (br s, 2H), 6.39 (dd, J=7.6, 4.8 Hz, 1H), 5.37-5.23 (m, 1H), 1.54 (d, J=6.8 Hz, 3H).

### Example 141: 5-(4-(4-(2-(2-Aminopyridin-3-yl)-5-(2-fluorophenyl)-3H-imidazo[4,5-b]pyridin-3-yl)benzyl)piperazine-1-carbonyl)-2-hydroxybenzaldehyde

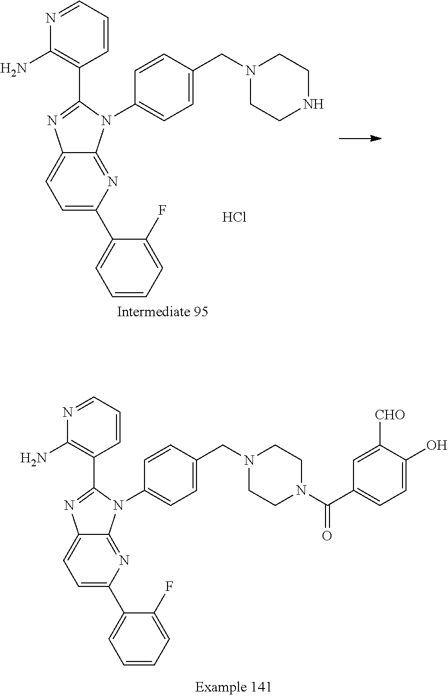 [SMILES: Nc1ncccc1-c1nc2ccc(-c3ccccc3F)nc2n1-c1ccc(CN2CCNCC2)cc1 ; Cl ; Nc1ncccc1-c1nc2ccc(-c3ccccc3F)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 95. After purified by prep-HPLC (column: Waters xbridge 150×25 mm×10 μm; mobile phase: [water (NH₄HCO₃)-ACN]; B %: 32%-62%, 10 min), 5-(4-(4-(2-(2-aminopyridin-3-yl)-5-(2-fluorophenyl)-3H-imidazo[4,5-b]pyridin-3-yl)benzyl)piperazine-1-carbonyl)-2-hydroxybenzaldehyde (Example 141, 19.3 mg, yield: 14%) was obtained as a yellow solid. MS: m/z=628.3 [M+H]⁺. ¹H NMR (400 MHz, Dimethylsulfoxide-d₆) δ 11.27 (br s, 1H), 10.27 (s, 1H), 8.29 (d, J=8.4 Hz, 11H), 8.01-7.96 (m, 1H), 7.83-7.74 (m, 2H), 7.68 (d, J=2.0 Hz, 1H), 7.56 (dd, J=8.4, 2.4 Hz, 1H), 7.49-7.43 (m, 5H), 7.35-7.27 (m, 2H), 7.15 (dd, J=8.0, 2.0 Hz, 1H), 7.04-6.99 (m, 3H), 6.38 (dd, J=7.6, 5.2 Hz, 1H), 3.60 (s, 2H), 3.56-3.40 (m, 4H), 2.46-2.38 (m, 4H). ¹⁹F NMR (400 MHz, Dimethylsulfoxide-d₆) δ −117.18.

### Example 142: 4-(4-(4-(2-(2-Aminopyridin-3-yl)-5-(2-fluorophenyl)-3H-imidazo[4,5-b]pyridin-3-yl)benzyl)piperazine-1-carbonyl)-2-hydroxybenzaldehyde

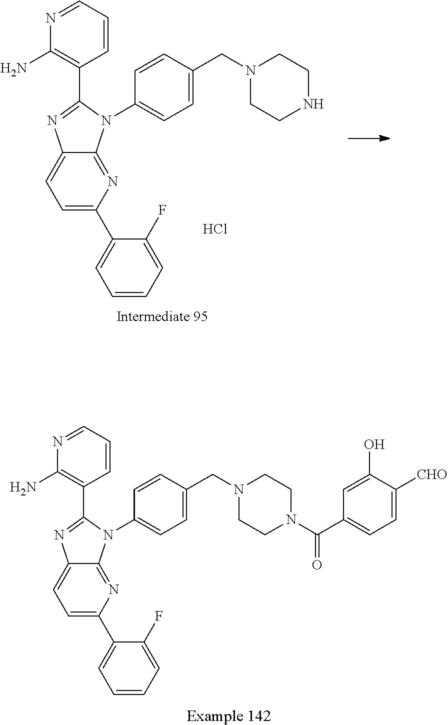 [SMILES: Nc1ncccc1-c1nc2ccc(-c3ccccc3F)nc2n1-c1ccc(CN2CCNCC2)cc1 ; Cl ; Nc1ncccc1-c1nc2ccc(-c3ccccc3F)nc2n1-c1ccc(CN2CCN(C(=O)c3ccc(C=O)c(O)c3)CC2)cc1]

Following the general procedures described in Example 36, the reaction was carried out using 4-formyl-3-hydroxybenzoic acid and Intermediate 95. The residue was purified by prep-HPLC (column: Waters xbridge 150×25 mm×10 μm; mobile phase: [water (NH₄HCO₃)-ACN]; B %: 35%-65%, 10 min), 4-(4-(4-(2-(2-Aminopyridin-3-yl)-5-(2-fluorophenyl)-3H-imidazo[4,5-b]pyridin-3-yl)benzyl)piperazine-1-carbonyl)-2-hydroxybenzaldehyde (Example 142, 32.1 mg, yield: 24%) was obtained as a yellow solid. MS: m/z=628.4 [M+H]⁺. ¹H NMR (400 MHz, Dimethylsulfoxide-d₆) δ 10.95 (br s, 1H), 10.29 (s, 1H), 8.29 (d, J=8.4 Hz, 1H), 8.03-7.96 (m, 1H), 7.82-7.76 (m, 2H), 7.69 (d, J=7.6 Hz, 1H), 7.49-7.43 (m, 5H), 7.35-7.29 (m, 2H), 7.19-7.14 (m, 1H), 6.98-6.89 (m, 4H), 6.39 (dd, J=7.6, 4.8 Hz, 1H), 3.60 (s, 2H), 3.27-3.17 (m, 4H), 2.41-2.36 (m, 4H). ¹⁹F NMR (400 MHz, Dimethylsulfoxide-d₆) δ −117.18.

### Example 143: 5-(4-(4-(2-(2-Aminopyridin-3-yl)-5-(4-fluorophenyl)-3H-imidazo[4,5-b]pyridin-3-yl)benzyl)piperazine-1-carbonyl)-2-hydroxybenzaldehyde

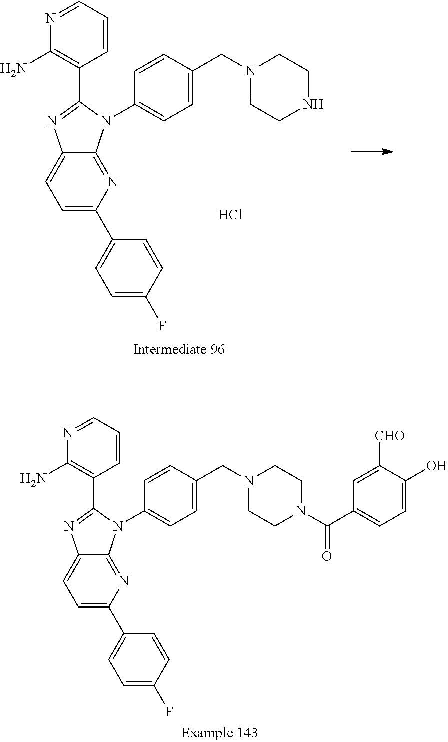 [SMILES: Nc1ncccc1-c1nc2ccc(-c3ccc(F)cc3)nc2n1-c1ccc(CN2CCNCC2)cc1 ; Cl ; Nc1ncccc1-c1nc2ccc(-c3ccc(F)cc3)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 96. After purified by prep-HPLC (column: Waters xbridge 150×25 mm×10 μm; mobile phase: [water (NH₄HCO₃)-ACN]; B %: 33%-63%, 10 min), 5-(4-(4-(2-(2-aminopyridin-3-yl)-5-(4-fluorophenyl)-3H-imidazo[4,5-b]pyridin-3-yl)benzyl)piperazine-1-carbonyl)-2-hydroxybenzaldehyde (Example 143, 16 mg, yield: 12%) was obtained as a yellow solid. MS: m/z=628.3 [M+H]⁺. ¹H NMR (400 MHz, Dimethylsulfoxide-d₆) δ 11.76-10.81 (m, 1H), 10.28 (s, 1H), 8.27 (d, J=8.4 Hz, 1H), 8.11-8.05 (m, 2H), 8.01-7.96 (m, 2H), 7.69 (d, J=2.4 Hz, 1H), 7.57 (dd, J=8.8, 2.4 Hz, 1H), 7.50-7.42 (m, 4H), 7.32-7.27 (m, 2H), 7.14 (dd, J=7.6, 1.6 Hz, 1H), 7.06-6.99 (m, 3H), 6.38 (dd, J=7.6, 4.8 Hz, 1H), 3.63 (s, 2H), 3.58-3.48 (m, 4H), 2.46-2.42 (m, 4H). ¹⁹F NMR (400 MHz, Dimethylsulfoxide-d₆) δ −113.57.

### Example 144: 4-(4-(4-(2-(2-Aminopyridin-3-yl)-5-(4-fluorophenyl)-3H-imidazo[4,5-b]pyridin-3-yl)benzyl)piperazine-1-carbonyl)-2-hydroxybenzaldehyde

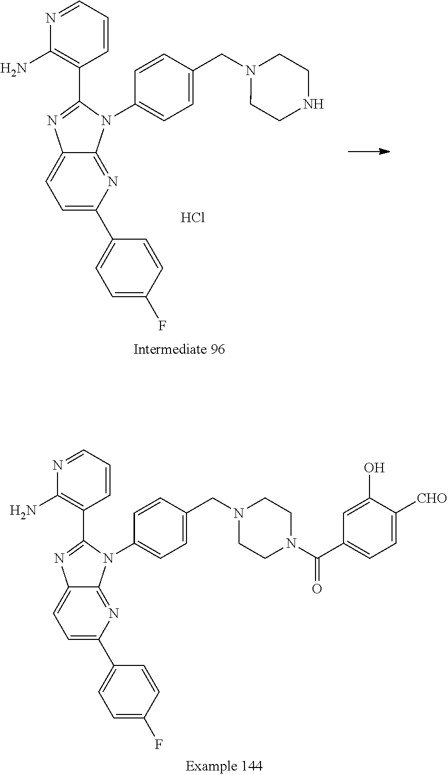 [SMILES: Nc1ncccc1-c1nc2ccc(-c3ccc(F)cc3)nc2n1-c1ccc(CN2CCNCC2)cc1 ; Cl ; Nc1ncccc1-c1nc2ccc(-c3ccc(F)cc3)nc2n1-c1ccc(CN2CCN(C(=O)c3ccc(C=O)c(O)c3)CC2)cc1]

Following the general procedures described in Example 36, the reaction was carried out using 3-formyl-4-hydroxybenzoic acid and Intermediate 96. After purified by prep-HPLC (column: Waters xbridge 150×25 mm×10 μm; mobile phase: [water (NH₄HCO₃)-ACN]; B %: 38%-68%, 10 min), 4-(4-(4-(2-(2-aminopyridin-3-yl)-5-(4-fluorophenyl)-3H-imidazo[4,5-b]pyridin-3-yl)benzyl)piperazine-1-carbonyl)-2-hydroxybenzaldehyde (Example 144, 17.2 mg, yield: 13%) was obtained as a yellow solid. MS: m/z=628.4 [M+H]⁺. ¹H NMR (400 MHz, Dimethylsulfoxide-d₆) δ 11.25-10.70 (m, 1H), 10.29 (s, 1H), 8.27 (d, J=8.4 Hz, 1H), 8.12-8.03 (m, 2H), 8.02-7.93 (m, 2H), 7.70 (d, J=7.6 Hz, 1H), 7.52-7.40 (m, 4H), 7.33-7.25 (m, 2H), 7.14 (dd, J=7.6, 1.6 Hz, 1H), 7.02 (s, 2H), 6.96-6.82 (m, 2H), 6.38 (dd, J=7.6, 4.8 Hz, 1H), 3.63 (s, 2H), 3.31-3.21 (m, 4H), 2.45-2.35 (m, 4H). ¹⁹F NMR (400 MHz, Dimethylsulfoxide-d₆) δ −113.57.

### Example 145: 4-(4-(4-(2-(2-Aminopyridin-3-yl)-5-(2-chlorophenyl)-3H-imidazo[4,5-b]pyridin-3-yl)benzyl)piperazin-1-yl)-2-hydroxybenzaldehyde

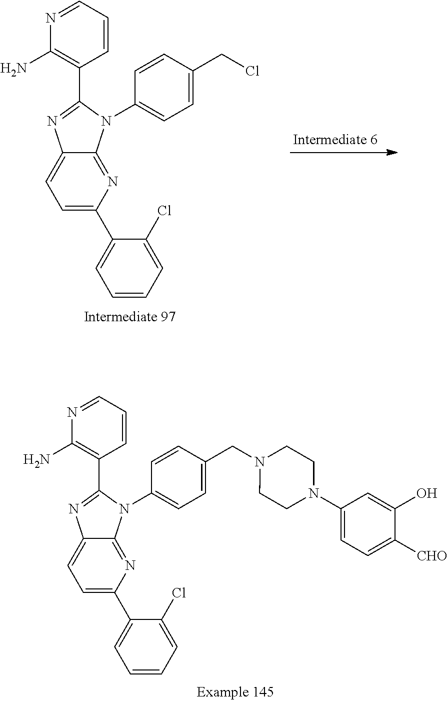 [SMILES: Nc1ncccc1-c1nc2ccc(-c3ccccc3Cl)nc2n1-c1ccc(CCl)cc1 ; Nc1ncccc1-c1nc2ccc(-c3ccccc3Cl)nc2n1-c1ccc(CN2CCN(c3ccc(C=O)c(O)c3)CC2)cc1]

Following the general procedures described in Example 73, the reaction was carried out using Intermediate 6 and Intermediate 97. After purified by prep-HPLC (column: Phenomenex C₁₈ 150×25 mm×10 μm; mobile phase: [water (NH₄HCO₃)-ACN]; B %: 60% 90%, over 14 min), 4-(4-(4-(2-(2-aminopyridin-3-yl)-5-(2-chlorophenyl)-3H-imidazo[4,5-b]pyridin-3-yl)benzyl)piperazin-1-yl)-2-hydroxybenzaldehyde (Example 145, 10.3 mg, yield: 15.9%) was obtained as a light yellow lyophilized powder. MS: m/z=616.4 [M+H]⁺. ¹H NMR (400 MHz, Dimethylsulfoxide-d₆)S 9.57 (s, 1H), 8.23 (d, J=8.0 Hz, 1H), 8.01-7.96 (m, 1H), 7.66 (d, J=8.4 Hz, 1H), 7.56-7.50 (m, 4H), 7.46-7.41 (m, 3H), 7.40-7.37 (m, 2H), 7.36-7.31 (m, 1H), 6.61-6.55 (m, 1H), 6.51-6.44 (m, 1H), 6.30 (d, J=1.6 Hz, 1H), 3.65 (s, 2H), 3.47-3.44 (m, 4H), 2.62-2.58 (m, 4H).

### Example 146: 4-(4-(4-(2-(2-Aminopyridin-3-yl)-5-(4-chlorophenyl)-3H-imidazo[4,5-b]pyridin-3-yl)benzyl)piperazin-1-yl)-2-hydroxybenzaldehyde

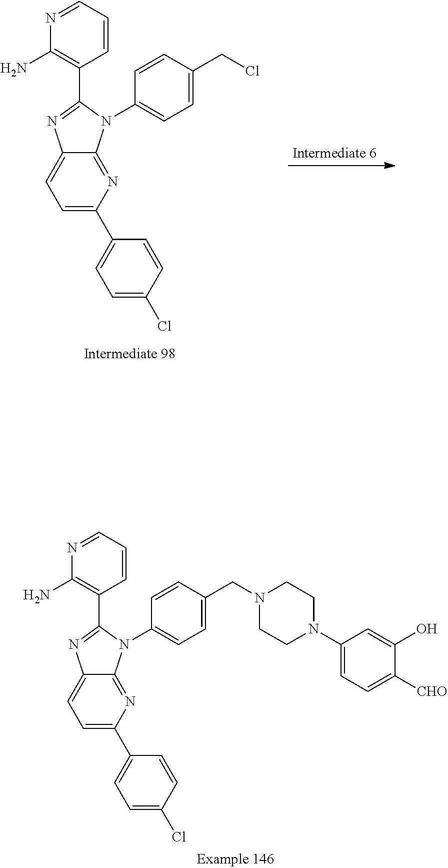 [SMILES: Nc1ncccc1-c1nc2ccc(-c3ccc(Cl)cc3)nc2n1-c1ccc(CCl)cc1 ; Nc1ncccc1-c1nc2ccc(-c3ccc(Cl)cc3)nc2n1-c1ccc(CN2CCN(c3ccc(C=O)c(O)c3)CC2)cc1]

Following the general procedures described in Example 73, the reaction was carried out using Intermediate 6 and Intermediate 98. After purified by prep-HPLC (column: Phenomenex C18 150×25 mm×10 μm; mobile phase: [water (NH₄HCO₃)-ACN]; B %: 60%-90%, over 14 min), 4-(4-(4-(2-(2-aminopyridin-3-yl)-5-(4-chlorophenyl)-3H-imidazo[4,5-b]pyridin-3-yl)benzyl)piperazin-1-yl)-2-hydroxybenzaldehyde (Example 146, 21.8 mg, yield: 24%) was obtained as a yellow lyophilized powder. MS: m/z=616.4 [M+H]⁺. ¹H NMR (400 MHz, Dimethylsulfoxide-d₆) δ 11.12 (s, 1H), 9.73 (s, 1H), 8.28 (d, J=8.4 Hz, 1H), 8.06 (d, J=8.4 Hz, 2H), 8.03-7.98 (m, 2H), 7.57-7.42 (m, 7H), 7.14 (dd, J=7.6, 1.6 Hz, 1H), 7.03 (br s, 2H), 6.61 (dd, J=8.8, 2.0 Hz, 1H), 6.36 (dd, J=7.6, 4.4 Hz, 1H), 6.33 (d, J=2.0 Hz, 1H), 3.64 (s, 2H), 3.47-3.40 (m, 4H), 2.56-2.52 (m, 4H).

### Example 147: 5-(4-(4-(2-(2-Aminopyridin-3-yl)-5-(4-chlorophenyl)-3H-imidazo[4,5-b]pyridin-3-yl)benzyl)piperazine-1-carbonyl)-2-hydroxybenzaldehyde

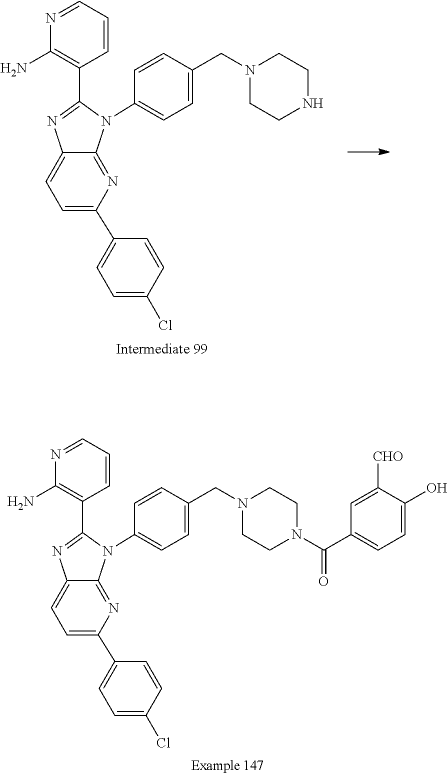 [SMILES: Nc1ncccc1-c1nc2ccc(-c3ccc(Cl)cc3)nc2n1-c1ccc(CN2CCNCC2)cc1 ; Nc1ncccc1-c1nc2ccc(-c3ccc(Cl)cc3)nc2n1-c1ccc(CN2CCN(C(=O)c3ccc(O)c(C=O)c3)CC2)cc1]

Following the general procedures described in Example 36, the reaction was carried out using 3-formyl-4-hydroxybenzoic acid and Intermediate 99. After purified by prep-HPLC (column: Phenomenex C18 150×25 mm×10 μm; mobile phase: [water (NH₄HCO₃)-ACN]; B %: 42%-72%, over 10 min), 5-(4-(4-(2-(2-aminopyridin-3-yl)-5-(4-chlorophenyl)-3H-imidazo[4,5-b]pyridin-3-yl)benzyl)piperazine-1-carbonyl)-2-hydroxybenzaldehyde (Example 147, 12.6 mg, yield: 14%) was obtained as a light-yellow lyophilized powder. MS: m/z=644.2 [M+H]⁺. ¹H NMR (400 MHz, Dimethylsulfoxide-d_{f};) δ 11.64-10.70 (m, 1H), 10.29 (s, 1H), 8.29 (d, J=8.4 Hz, 1H), 8.08-7.99 (m, 4H), 7.69 (d, J=2.0 Hz, 1H), 7.58 (dd, J=8.4, 2.0 Hz, 1H), 7.54-7.49 (m, 4H), 7.48-7.42 (m, 2H), 7.15 (dd, J=7.6, 1.6 Hz, 1H), 7.07-7.00 (m, 3H), 6.39 (dd, J=7.6, 4.8 Hz, 1H), 3.64 (s, 2H), 3.59-3.48 (m, 4H), 2.48-2.42 (m, 4H).

### Example 148: 4-(4-(4-(2-(2-Aminopyridin-3-yl)-5-(4-chlorophenyl)-3H-imidazo[4,5-b]pyridin-3-yl)benzyl)piperazine-1-carbonyl)-2-hydroxybenzaldehyde

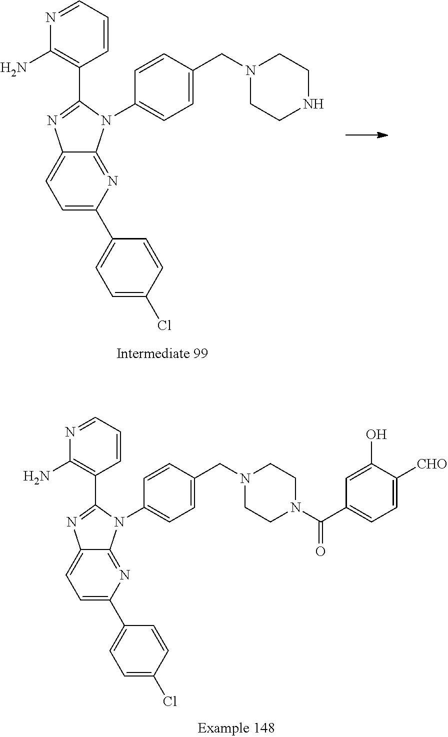 [SMILES: Nc1ncccc1-c1nc2ccc(-c3ccc(Cl)cc3)nc2n1-c1ccc(CN2CCNCC2)cc1 ; Nc1ncccc1-c1nc2ccc(-c3ccc(Cl)cc3)nc2n1-c1ccc(CN2CCN(C(=O)c3ccc(C=O)c(O)c3)CC2)cc1]

Following the general procedures described in Example 36, the reaction was carried out using 4-formyl-3-hydroxybenzoic acid and Intermediate 99. After purified by prep-HPLC (column: Phenomenex C18 150×25 mm×10 μm; mobile phase: [water (NH₄HCO₃)-ACN]; B %: 45%-75%, over 10 min), 4-(4-(4-(2-(2-aminopyridin-3-yl)-5-(4-chlorophenyl)-3H-imidazo[4,5-b]pyridin-3-yl)benzyl)piperazine-1-carbonyl)-2-hydroxybenzaldehyde (Example 148, 21.8 mg, yield: 24%) was obtained as a light-yellow solid. MS: m/z=644.2 [M+H]⁺. ¹H NMR (400 MHz, Dimethylsulfoxide-d₆) δ 11.24-10.69 (m, 1H), 10.30 (s, 1H), 8.29 (d, J=8.4 Hz, 1H), 8.08-7.98 (m, 4H), 7.70 (d, J=7.6 Hz, 1H), 7.54-7.45 (m, 6H), 7.15 (d, J=7.6 Hz, 1H), 7.02 (br s, 2H), 6.98-6.89 (m, 2H), 6.39 (dd, J=7.2, 5.2 Hz, 1H), 3.73-3.63 (m, 4H), 3.37-3.35 (m, 2H), 2.49-2.36 (m, 4H).

### Example 149: 5-(4-(4-(2-(2-Aminopyridin-3-yl)-5-(4-chlorophenyl)-3H-imidazo[4,5-b]pyridin-3-yl)benzyl)piperazin-1-yl)-2-hydroxybenzaldehyde

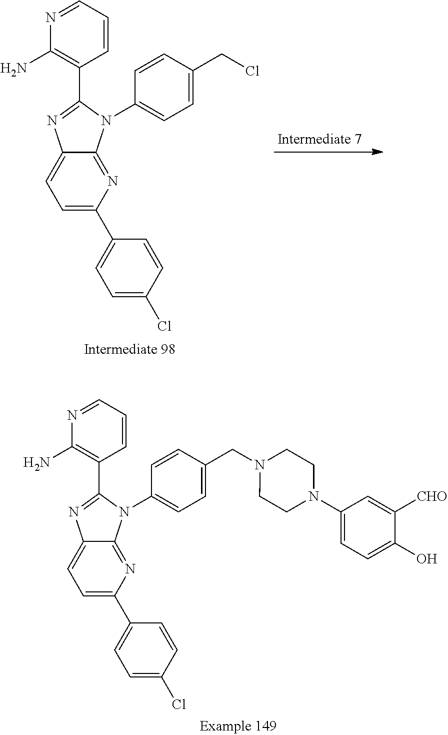 [SMILES: Nc1ncccc1-c1nc2ccc(-c3ccc(Cl)cc3)nc2n1-c1ccc(CCl)cc1 ; Nc1ncccc1-c1nc2ccc(-c3ccc(Cl)cc3)nc2n1-c1ccc(CN2CCN(c3ccc(O)c(C=O)c3)CC2)cc1]

Following the general procedures described in Example 73, the reaction was carried out using Intermediate 98 and Intermediate 7. After purified by prep-HPLC (column: Waters xbridge 150×25 mm×10 μm; mobile phase: [water (NH₄HCO₃)-ACN]; gradient: 59%-89% B over 14 min) and prep-TLC (CH₂Cl₂:MeOH=10:1), 5-(4-(4-(2-(2-aminopyridin-3-yl)-5-(4-chlorophenyl)-3H-imidazo[4,5-b]pyridin-3-yl)benzyl)piperazin-1-yl)-2-hydroxybenzaldehyde (Example 149, 3.7 mg, yield: 3.3%) was obtained as a light-yellow solid. MS: m/z=616.1 [M+H]⁺. ¹H NMR (400 MHz, Methanol-d₄) δ 10.03 (s, 1H), 8.22 (d, J=8.4 Hz, 1H), 8.04-7.96 (m, 4H), 7.66 (d, J=7.6 Hz, 2H), 7.55 (d, J=8.0 Hz, 2H), 7.43 (d, J=8.4 Hz, 2H), 7.38-7.28 (m, 3H), 6.92 (d, J=8.8 Hz, 1H), 6.51 (dd, J=7.6, 4.8 Hz, 1H), 4.05 (br s, 2H), 3.27-3.18 (m, 4H), 3.12-2.93 (m, 4H).

### Example 150: 5-(4-(4-(2-(2-Aminopyridin-3-yl)-5-(cyclohex-1-en-1-yl)-3H-imidazo[4,5-b]pyridin-3-yl)benzyl)piperazine-1-carbonyl)-2-hydroxybenzaldehyde

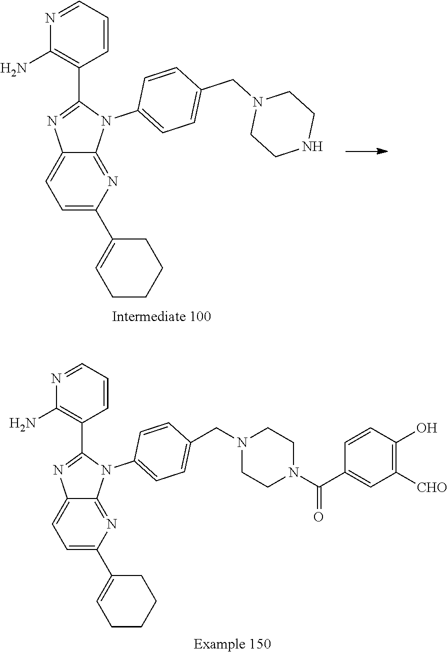 [SMILES: Nc1ncccc1-c1nc2ccc(C3=CCCCC3)nc2n1-c1ccc(CN2CCNCC2)cc1 ; Nc1ncccc1-c1nc2ccc(C3=CCCCC3)nc2n1-c1ccc(CN2CCN(C(=O)c3ccc(O)c(C=O)c3)CC2)cc1]

Following the general procedures described in Example 44, the reaction was carried out using 3-formyl-4-hydroxybenzoic acid and Intermediate 100. After purified by prep-HPLC (column: Welch Xtimate C18 150×25 mm×5 μm; mobile phase: [water (NH₃H₂O+NH₄HCO₃)-ACN]; gradient: 50%-80% B over 7 min) and (column: Phenomenex C18 75×30 mm×3 μm; mobile phase: [water (NH₃H₂O+NH₄HCO₃)-ACN]; gradient: 37%-67% B over 8 min), 5-(4-(4-(2-(2-aminopyridin-3-yl)-5-(cyclohex-1-en-1-yl)-3H-imidazo[4,5-b]pyridin-3-yl)benzyl)piperazine-1-carbonyl)-2-hydroxybenzaldehyde (Example 150, 4.9 mg, yield: 1.3%) was obtained as a yellow solid. MS: m/z=614.2 [M+H]⁺. ¹H NMR (400 MHz, Chloroform-d) δ 11.19 (br s, 1H), 9.93 (s, 1H), 8.05-7.92 (m, 2H), 7.75 (d, J=2.0 Hz, 1H), 7.61 (dd, J=8.8, 2.0 Hz, 1H), 7.55-7.42 (m, 4H), 7.41-7.33 (m, 3H), 7.16 (d, J=7.2 Hz, 1H), 7.03 (d, J=8.8 Hz, 1H), 6.74-6.69 (m, 1H), 6.42-6.36 (m, 1H), 3.80-3.60 (m, 5H), 2.62-2.47 (m, 6H), 2.31-2.18 (m, 3H), 1.79-1.73 (m, 2H), 1.70-1.63 (m, 2H).

### Example 151: 4-(4-(4-(2-(2-Aminopyridin-3-yl)-5-(cyclohex-1-en-1-yl)-3H-imidazo[4,5-b]pyridin-3-yl)benzyl)piperazine-1-carbonyl)-2-hydroxybenzaldehyde

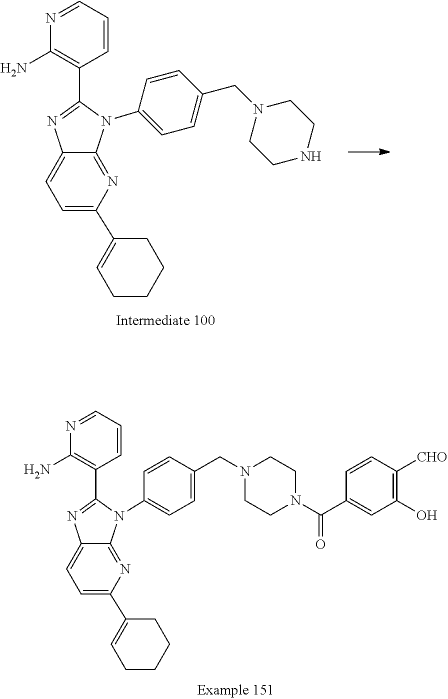 [SMILES: Nc1ncccc1-c1nc2ccc(C3=CCCCC3)nc2n1-c1ccc(CN2CCNCC2)cc1 ; Nc1ncccc1-c1nc2ccc(C3=CCCCC3)nc2n1-c1ccc(CN2CCN(C(=O)c3ccc(C=O)c(O)c3)CC2)cc1]

Following the general procedures described in Example 44, the reaction was carried out using 4-formyl-3-hydroxybenzoic acid and Intermediate 100. After purified by prep-HPLC (column: Welch Xtimate C18 150×25 mm×5 μm; mobile phase: [water (NH₃H₂O+NH₄HCO₃)-ACN]; gradient: 60%-90% B over 7 min) and prep-HPLC (column: Phenomenex C18 80×40 mm×3 μm; mobile phase: [water (NH₃H₂O+NH₄HCO₃)-ACN]; gradient: 50%-80% B over 7 min), 4-(4-(4-(2-(2-aminopyridin-3-yl)-5-(cyclohex-1-en-1-yl)-3H-imidazo[4,5-b]pyridin-3-yl)benzyl)piperazine-1-carbonyl)-2-hydroxybenzaldehyde (Example 151, 5.2 mg, yield: 1.6%) was obtained as a yellow solid. MS: m/z=614.1 [M+H]⁺. ¹H NMR (400 MHz, Chloroform-d) 11.10 (s, 1H), 9.93 (s, 1H), 8.03 (d, J=8.4 Hz, 1H), 7.92 (d, J=5.2 Hz, 1H), 7.82-7.74 (m, 1H), 7.64 (d, J=7.6 Hz, 1H), 7.53 (d, J=8.0 Hz, 2H), 7.47 (d, J=8.4 Hz, 1H), 7.37 (d, J=8.4 Hz, 2H), 7.22 (d, J=8.2 Hz, 1H), 7.12-7.03 (m, 2H), 7.02-6.97 (m, 1H), 6.75-6.69 (m, 1H), 6.41 (dd, J=8.0, 5.6 Hz, 1H), 3.91-3.80 (m, 2H), 3.69 (s, 2H), 3.52-3.42 (m, 2H), 2.67-2.63 (m, 1H), 2.55-2.47 (m, 4H), 2.32-2.20 (m, 3H), 1.79-1.73 (m, 2H), 1.69-1.63 (m, 2H).

### Example 152: 4-(9-(4-(2-(2-Aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde

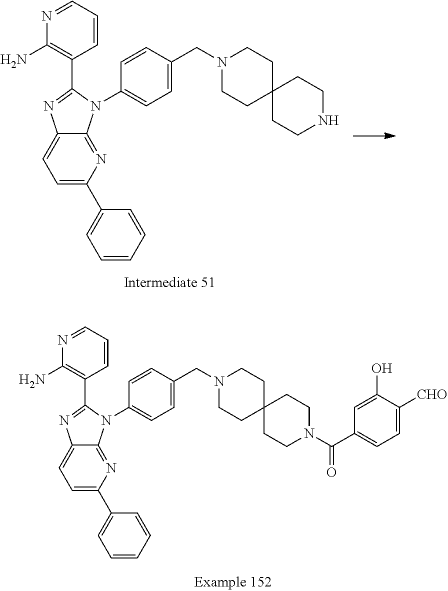 [SMILES: Nc1ncccc1-c1nc2ccc(-c3ccccc3)nc2n1-c1ccc(CN2CCC3(CCNCC3)CC2)cc1 ; Nc1ncccc1-c1nc2ccc(-c3ccccc3)nc2n1-c1ccc(CN2CCC3(CC2)CCN(C(=O)c2ccc(C=O)c(O)c2)CC3)cc1]

Following the general procedures described in Example 44, the reaction was carried out using 4-formyl-3-hydroxybenzoic acid and Intermediate 51. After purified by prep-HPLC (column: Phenomenex C18 150×25 mm×10 μm; mobile phase: [water (NH₄HCO₃)-ACN], gradient: 37%-67% B over 14 min), 4-(9-(4-(2-(2-aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde (Example 152, 18.7 mg, yield: 16%) was obtained as a yellow lyophilized powder. MS: m/z=678.4 [M+H]⁺. ¹H NMR (400 MHz, Dimethylsulfoxide-d₆) S 11.07-10.74 (m, 1H), 10.28 (s, 1H), 8.27 (d, J=8.4 Hz, 1H), 8.06-7.92 (m, 4H), 7.69 (d, J=7.6 Hz, 1H), 7.49-7.35 (m, 7H), 7.20-7.12 (m, 1H), 7.02 (s, 2H), 6.99-6.85 (m, 2H), 6.47-6.30 (m, 1H), 3.68-3.53 (m, 4H), 3.28-3.21 (m, 2H), 2.46-2.32 (m, 4H), 1.57-1.43 (m, 6H), 1.42-1.30 (m, 2H).

### Example 153: 4-(6-(4-(2-(2-Aminopyridin-3-yl)-5-phenyl-3H-imidazo[4,5-b]pyridin-3-yl)benzyl)-2,6-diazaspiro[3.4]octane-2-carbonyl)-2-hydroxybenzaldehyde

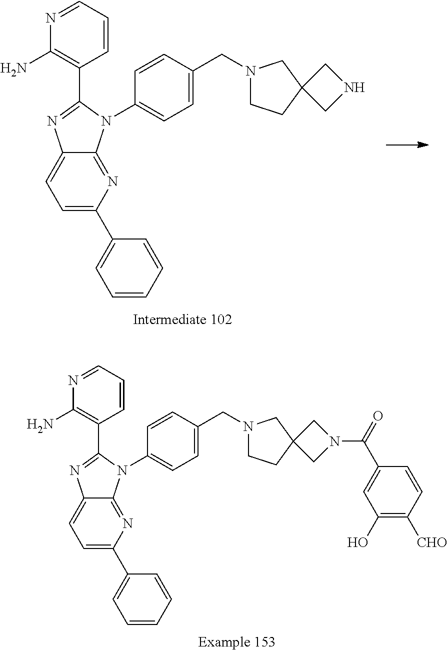

Following the general procedures described in Example 44, the reaction was carried out using 4-formyl-3-hydroxybenzoic acid and Intermediate 102. After purified by prep-HPLC (column: Phenomenex C18 150×25 mm×10 μm; mobile phase: [water(NH₄HCO₃)-ACN]; gradient: 30%-60% B over 14 min), 4-(6-(4-(2-(2-aminopyridin-3-yl)-5-phenyl-3H-imidazo[4,5-b]pyridin-3-yl)benzyl)-2,6-diazaspiro[3.4]octane-2-carbonyl)-2-hydroxybenzaldehyde (Example 153, 11 mg, yield: 12%) was obtained as a yellow lyophilized powder. MS: m/z=636.4 [M+H]⁺. 1H NMR (400 MHz, Methanol-d₄) 10.04 (s, 1H), 8.19 (d, J=8.4 Hz, 1H), 8.04-8.00 (m, 2H), 7.97-7.91 (m, 2H), 7.77 (d, J=7.6 Hz, 1H), 7.56-7.51 (m, 2H), 7.47-7.43 (m, 2H), 7.44-7.38 (m, 2H), 7.38-7.35 (m, 1H), 7.34-7.30 (m, 1H), 7.25-7.14 (m, 2H), 6.51-6.40 (m, 1H), 4.27 (s, 2H), 4.15-4.08 (m, 2H), 3.75 (s, 2H), 2.92-2.81 (m, 2H), 2.75-2.70 (m, 2H), 2.21-2.16 (m, 2H).

### II. Biological Evaluation

### Example 1: NanoBRET Target Engagement (TE) Assay

NanoBRET is a highly specific and validated cell-based technique for assessing target engagement (Vasta et al., 2018, Cell Chem Biol. 25(2):206-214). The NanoBRET™ Target Engagement (TE) Intracellular Kinase Assays are based on the NanoBRET™ System (Promega Corporation), an energy transfer technique designed to measure molecular proximity in living cells. The NanoBRET™ TE Assays measure the apparent affinity of test compounds by competitive displacement of the NanoBRET™ tracer compound, which is a cell permeable molecule engineered to be reversibly bound to a NanoLuc® luciferase-kinase fusion expressed in cells. For compound screening, when a test compound binds to the selected kinase, the BRET signal is attenuated. For kinase inhibitors in particular, intracellular target selectivity is fundamental to pharmacological mechanism and allows the proteins of interest to be in the correct cellular confirmation. Although non-cell-based techniques have been developed to measure kinase binding or enzymatic inhibition with accuracy and precision, such approaches can fail to accurately predict engagement of the full-length target protein in the more complex and biologically relevant cellular context (Knight and Shokat, 2005, Chem. Biol. 12, 621-637; Smyth and Collins, 2009, J. Chem. Biol. 2, 131-151). The NanoBRET assay procedure was used to interrogate the compounds against the full length AKT E17K per manufacturers suggestions. Briefly, HEK-293 cells (ATCC Cat #CRL-1573) were used for transfection purposes using FuGENE HD Transfection Reagent (Promega Cat #E2311). All cells were evaluated for viability prior to transfection and optimization of the transfection was done prior to experimentation. Greater than 95% viability was used for all experiments. Following transfection, cells were washed and resuspended in Opti-MEM. NanoBRET assays were performed in white, 384-well plates (Corning) at a density of 2×10⁵ cells/well. All example compounds were prepared as concentrated stock solutions in DMSO (Sigma-Aldrich). Compounds are dissolved in DMSO to make 10 mM stock solution. Example compounds were transferred as 40 μL of 10 mM stock solution to a 384 pp-plate (LABCYTE, PP-0200) and diluted in 3-fold, 10-point dilution via transferring 12 μL compound into 24 μL DMSO by Apricot liquid handler. A Labcyte ECHO 550 compound dispenser was used to facilitate compound transfer directly to cells. Cells were equilibrated for 2 hr with energy transfer probes and example compound prior to BRET measurements. The AKTE17K (Promega Cat #NV2421) as well as specific probe (NanoBRET tracer, Promega Cat #N264B) was prepared at a concentration of 20× in tracer dilution buffer (12.5 mM HEPES, 31.25% PEG-400, pH 7.5). For target engagement analysis, the energy transfer probes were added to the cells at concentrations optimized for the target in question (AKT E17K). Following compound incubation, NanoBRET NanoGlo Substrate (Promega Cat #N157D) and Extracellular Nanoluc Inhibitor (Promega Cat #N235C) was added according to the manufacturer's recommended protocol, and luminescence was measured on Envision Reader (Perkin Elmer) Multimode Luminometer equipped with 450 nmBPfilter (donor) and 600 nmLPfilter (acceptor), using 0.5 s integration time. Milli-BRET units (mBU) are calculated by multiplying the raw BRET values by 1000. Apparent tracer affinity values (EC50) were determined using the sigmoidal dose-response (variable slope). Competitive displacement data were then plotted and data were fit to determine the EC₅₀ value for each example compound. Table 3 provides the assay results for select examples. Activity is defined as “+”, for EC₅₀ greater 600 nanomolar; “++” for EC₅₀ between 60-600 nanomolar; “+++” for EC₅₀ between 15-60 nanomolar; and “++++”, for EC₅₀ less than 15 nanomolar.

| Ex- | | AKT1- |
|---|---|---|
| am- | | E17K |
| ple | Name | IC₅₀ |
| 1 | N-(4-(2-(2-aminopyridin-3-yl)-5- | ++ |
| | phenyl-3H-imidazo[4,5-b]pyridin-3- | |
| | yl)benzyl)-4-formyl-3-hydroxybenzamide | |
| 2 | N-(4-(2-(2-aminopyridin-3-yl)- | ++ |
| | 3H-imidazo[4,5-b]pyridin-3- | |
| | yl)benzyl)-3-formyl-4-hydroxybenzamide | |
| 3 | N-(4-(2-(2-aminopyridin-3-yl)- | + |
| | 3H-imidazo[4,5-b]pyridin-3- | |
| | yl)benzyl)-4-formyl-3-hydroxybenzamide | |
| 4 | N-(4-(2-(2-aminopyridin-3-yl)-5- | +++ |
| | phenyl-3H-imidazo[4,5-b]pyridin- | |
| | 3-yl)benzyl)-3-formyl-4-hydroxybenzamide | |
| 5 | 5-(4-(4-(2-(2-aminopyridin-3- | ++ |
| | yl)-5-phenyl-3H-imidazo[4,5- | |
| | b]pyridin-3-yl)benzyl)piperazine-1-carbonyl)-2- | |
| | hydroxybenzaldehyde | |
| 6 | 4-(4-(4-(2-(2-Aminopyridin-3-yl)-5-phenyl-3H- | ++ |
| | imidazo[4,5-b]pyridin-3-yl)benzyl)piperazine-1- | |
| | carbonyl)-2-hydroxybenzaldehyde | |
| 7 | N-(1-(4-(2-(2-aminopyridin-3-yl)-5-phenyl-3H- | ++++ |
| | imidazo[4,5-b]pyridin-3-yl)benzyl)piperidin- | |
| | 4-yl)-3-formyl-4-hydroxybenzamide | |
| 8 | N-(1-(4-(2-(2-aminopyridin-3-yl)-5-phenyl-3H- | ++ |
| | imidazo[4,5-b]pyridin-3-yl)benzyl)piperidin- | |
| | 4-yl)-4-formyl-3-hydroxybenzamide | |
| 9 | 4-(4-(4-(2-(2-aminopyridin-3-yl)-5-phenyl- | +++ |
| | 3H-imidazo[4,5-b]pyridin- | |
| | 3-yl)benzyl)piperazin- | |
| | 1-yl)-2-hydroxybenzaldehyde | |
| 10 | 5-(4-(4-(2-(2-aminopyridin-3-yl)-5-phenyl- | ++ |
| | 3H-imidazo[4,5-b]pyridin- | |
| | 3-yl)benzyl)piperazin- | |
| | 1-yl)-2-hydroxybenzaldehyde | |
| 11 | N-(1-(4-(2-(2-aminopyridin-3- | + |
| | yl)-5-phenyl-3H-imidazo[4,5-b]pyridin- | |
| | 3-yl)phenyl)cyclobutyl)-3- | |
| | formyl-4-hydroxybenzamide | |
| 12 | N-(1-(4-(2-(2-aminopyridin-3-yl)-5- | ++ |
| | phenyl-3H-imidazo[4,5-b]pyridin-3- | |
| | yl)benzyl)piperidin-4-yl)-N-cyclopropyl-3- | |
| | formyl-4-hydroxybenzamide | |
| 13 | N-(1-(4-(2-(2-aminopyridin-3-yl)-5-phenyl-3H- | ++ |
| | imidazo[4,5-b]pyridin-3-yl)benzyl)piperidin-4- | |
| | yl)-N-ethyl-3-formyl-4-hydroxybenzamide | |
| 14 | 5-(4-(4-(2-(2-aminopyridin-3-yl)-5-(2-oxo- | + |
| | 1,2-dihydropyridin-3-yl)-3H-imidazo[4,5- | |
| | b]pyridin-3-yl)benzyl)piperazine-1- | |
| | carbonyl)-2-hydroxybenzaldehyde | |
| 15 | 4-(4-(4-(2-(2-aminopyridin-3-yl)-5-(2-oxo- | + |
| | 1,2-dihydropyridin-3-yl)-3H-imidazo[4,5- | |
| | b]pyridin-3-yl)benzyl)piperazine-1- | |
| | carbonyl)-2-hydroxybenzaldehyde | |
| 16 | 4-(4-(4-(2-(2-aminopyridin-3-yl)-3H- | ++ |
| | imidazo[4,5-b]pyridin-3-yl)benzyl)piperazin- | |
| | 1-yl)-2-hydroxybenzaldehyde | |
| 17 | 4-((4-(2-(2-aminopyridin-3-yl)- | |
| | 5-phenyl-3H-imidazo[4,5- | ++ |
| | b]pyridin-3-yl)benzyl)(methyl)amino)- | |
| | 2-hydroxybenzaldehyde | |
| 18 | 5-((4-(2-(2-aminopyridin-3- | + |
| | yl)-5-phenyl-3H-imidazo[4,5- | |
| | b]pyridin-3-yl)benzyl)(methyl)amino)- | |
| | 2-hydroxybenzaldehyde | |
| 19 | N-(4-(2-(2-aminopyridin-3-yl)-5-phenyl- | ++ |
| | 3H-imidazo[4,5-b]pyridin-3-yl)benzyl)- | |
| | 5-formyl-2-hydroxybenzamide | |
| 20 | N-(4-(2-(2-aminopyridin-3-yl)-5-phenyl- | + |
| | 3H-imidazo[4,5-b]pyridin-3-yl)benzyl)- | |
| | 3-formyl-2-hydroxybenzamide | |
| 21 | N-(4-(2-(2-aminopyridin-3-yl)-5-phenyl- | ++ |
| | 3H-imidazo[4,5-b]pyridin-3-yl)benzyl)- | |
| | 2-formyl-3-hydroxybenzamide | |
| 22 | 2-amino-N-(4-(2-(2-aminopyridin-3-yl)- | + |
| | 5-phenyl-3H-imidazo[4,5-b]pyridin-3- | |
| | yl)benzyl)-5-formyl-4-hydroxybenzamide | |
| 23 | 3-(4-(4-(2-(2-aminopyridin- | ++ |
| | 3-yl)-5-phenyl-3H-imidazo[4,5- | |
| | b]pyridin-3-yl)benzyl)piperazin-1-yl)- | |
| | 2-hydroxybenzaldehyde | |
| 24 | 4-(4-(4-(2-(2-aminopyridin-3- | ++ |
| | yl)-5-phenyl-3H-imidazo[4,5- | |
| | b]pyridin-3-yl)benzyl)piperazin-1- | |
| | yl)-2-fluoro-6-hydroxybenzaldehyde | |
| 25 | 4-(4-(4-(2-(2-aminopyridin-3- | +++ |
| | yl)-5-phenyl-3H-imidazo[4,5- | |
| | b]pyridin-3-yl)benzyl)piperazin-1-yl)- | |
| | 5-fluoro-2-hydroxybenzaldehyde | |
| 26 | 4-(4-(4-(2-(2-aminopyridin- | +++ |
| | 3-yl)-5-phenyl-3H-imidazo[4,5-b]pyridin- | |
| | 3-yl)benzyl)piperazin-1-yl)-3-fluoro-2- | |
| | hydroxybenzaldehyde | |
| 27 | 4-(4-(4-(2-(2-aminopyridin-3-yl)-5- | ++ |
| | phenyl-3H-imidazo[4,5-b]pyridin-3- | |
| | yl)benzyl)piperazin-1-yl)-5-bromo-2- | |
| | hydroxybenzaldehyde | |
| 28 | 4-(4-(4-(2-(2-aminopyridin-3-yl)- | ++ |
| | 5-phenyl-3H-imidazo[4,5-b]pyridin- | |
| | 3-yl)benzyl)piperazin-1-yl)-2-hydroxy-5- | |
| | vinylbenzaldehyde | |
| 29 | 5-(4-(4-(2-(2-aminopyridin-3-yl)-5- | ++ |
| | (3,6-dihydro-2H-pyran-4-yl)-3H-imidazo[4,5- | |
| | b]pyridin-3-yl)benzyl)piperazine-1- | |
| | carbonyl)-2-hydroxybenzaldehyde | |
| 30 | 4-(4-(4-(2-(2-aminopyridin-3-yl)-5-(3,6- | ++ |
| | dihydro-2H-pyran-4-yl)-3H-imidazo[4,5- | |
| | b]pyridin-3-yl)benzyl)piperazine-1- | |
| | carbonyl)-2-hydroxybenzaldehyde | |
| 31 | 4-(4-(4-(2-(2-aminopyridin-3- | ++ |
| | yl)-5-morpholino-3H-imidazo[4,5- | |
| | b]pyridin-3-yl)benzyl)piperazin- | |
| | 1-yl)-2-hydroxybenzaldehyde | |
| 32 | 5-(4-(4-(2-(2-aminopyridin-3- | + |
| | yl)-5-morpholino-3H-imidazo[4,5- | |
| | b]pyridin-3-yl)benzyl)piperazine-1- | |
| | carbonyl)-2-hydroxybenzaldehyde | |
| 33 | 4-(4-(4-(2-(2-aminopyridin-3-yl)- | + |
| | 5-morpholino-3H-imidazo[4,5- | |
| | b]pyridin-3-yl)benzyl)piperazine- | |
| | 1-carbonyl)-2-hydroxybenzaldehyde | |
| 34 | 3-(2-(2-aminopyridin-3-yl)-3-(4-((4-(3- | ++ |
| | formyl-4-hydroxybenzoyl)piperazin- | |
| | 1-yl)methyl)phenyl)-3H- | |
| | imidazo[4,5-b]pyridin-5-yl)benzonitrile | |
| 35 | 3-(2-(2-aminopyridin-3-yl)-3-(4- | ++ |
| | ((4-(4-formyl-3-hydroxybenzoyl)piperazin- | |
| | 1-yl)methyl)phenyl)-3H- | |
| | imidazo[4,5-b]pyridin-5-yl)benzonitrile | |
| 36 | 2-(2-(2-aminopyridin-3-yl)-3-(4- | + |
| | ((4-(3-formyl-4-hydroxybenzoyl)piperazin- | |
| | 1-yl)methyl)phenyl)-3H- | |
| | imidazo[4,5-b]pyridin-5-yl)benzonitrile | |
| 37 | 2-(2-(2-aminopyridin-3-yl)-3-(4-((4- | ++ |
| | (4-formyl-3-hydroxybenzoyl)piperazin- | |
| | 1-yl)methyl)phenyl)- | |
| | 3H-imidazo[4,5-b]pyridin-5-yl)benzonitrile | |
| 38 | 4-(2-(2-aminopyridin-3-yl)-3-(4- | ++ |
| | ((4-(3-formyl-4-hydroxybenzoyl)piperazin- | |
| | 1-yl)methyl)phenyl)-3H- | |
| | imidazo[4,5-b]pyridin-5-yl)benzonitrile | |
| 39 | 4-(2-(2-aminopyridin-3-yl)-3-(4-((4- | ++ |
| | (4-formyl-3-hydroxybenzoyl)piperazin- | |
| | 1-yl)methyl)phenyl)-3H- | |
| | imidazo[4,5-b]pyridin-5-yl)benzonitrile | |
| 40 | 5-(4-(4-(2-(2-aminopyridin- | ++ |
| | 3-yl)-5-(3-fluorophenyl)-3H- | |
| | imidazo[4,5-b]pyridin-3-yl)benzyl)piperazine- | |
| | 1-carbonyl)-2-hydroxybenzaldehyde | |
| 41 | 4-(4-(4-(2-(2-aminopyridin- | ++ |
| | 3-yl)-5-(3-fluorophenyl)-3H- | |
| | imidazo[4,5-b]pyridin-3-yl)benzyl)piperazine- | |
| | 1-carbonyl)-2-hydroxybenzaldehyde | |
| 42 | 5-(4-(4-(2-(2-aminopyridin-3- | ++ |
| | yl)-5-(3-chlorophenyl)-3H- | |
| | imidazo[4,5-b]pyridin-3-yl)benzyl)piperazine- | |
| | 1-carbonyl)-2-hydroxybenzaldehyde | |
| 43 | 4-(4-(4-(2-(2-aminopyridin-3- | ++ |
| | yl)-5-(3-chlorophenyl)-3H- | |
| | imidazo[4,5-b]pyridin-3-yl)benzyl)piperazine- | |
| | 1-carbonyl)-2-hydroxybenzaldehyde | |
| 44 | 5-(4-(4-(2-(2-aminopyridin-3- | + |
| | yl)-5-(1-methyl-6-oxo-1,6-dihydropyridin- | |
| | 3-yl)-3H-imidazo[4,5-b]pyridin-3- | |
| | yl)benzyl)piperazine-1-carbonyl)- | |
| | 2-hydroxybenzaldehyde | |
| 45 | 5-(4-(4-(2-(2-aminopyridin-3-yl)-5-(6-oxo- | + |
| | 1,6-dihydropyridin-2-yl)-3H-imidazo[4,5- | |
| | b]pyridin-3-yl)benzyl)piperazine-1- | |
| | carbonyl)-2-hydroxybenzaldehyde | |
| 46 | 4-(4-(4-(2-(2-aminopyridin-3-yl)-5-(6-oxo- | + |
| | 1,6-dihydropyridin-2-yl)-3H-imidazo[4,5- | |
| | b]pyridin-3-yl)benzyl)piperazine-1- | |
| | carbonyl)-2-hydroxybenzaldehyde | |
| 47 | 4-((1-(4-(2-(2-aminopyridin-3-yl)-5-phenyl-3H- | ++ |
| | imidazo[4,5-b]pyridin-3-yl)benzyl)piperidin-4- | |
| | yl)amino)-2-hydroxybenzaldehyde | |
| 48 | 5-((1-(4-(2-(2-aminopyridin-3-yl)-5-phenyl-3H- | ++ |
| | imidazo[4,5-b]pyridin-3-yl)benzyl)piperidin-4- | |
| | yl)amino)-2-hydroxybenzaldehyde | |
| 49 | N-(1-(4-(2-(2-aminopyridin-3-yl)-5-phenyl-3H- | +++ |
| | imidazo[4,5-b]pyridin-3-yl)benzyl)piperidin- | |
| | 4-yl)-3-formyl-2-hydroxybenzamide | |
| 50 | 3-((1-(4-(2-(2-aminopyridin-3-yl)-5-phenyl-3H- | +++ |
| | imidazo[4,5-b]pyridin-3-yl)benzyl)piperidin- | |
| | 4-yl)amino)-2-hydroxybenzaldehyde | |
| 51 | 2-((1-(4-(2-(2-aminopyridin-3-yl)-5-phenyl-3H- | ++ |
| | imidazo[4,5-b]pyridin-3-yl)benzyl)piperidin-4- | |
| | yl)amino)-6-hydroxybenzaldehyde | |
| 52 | N-(1-(4-(2-(2-aminopyridin-3- | +++ |
| | yl)-5-morpholino-3H-imidazo[4,5- | |
| | b]pyridin-3-yl)benzyl)piperidin- | |
| | 4-yl)-3-formyl-4-hydroxybenzamide | |
| 53 | N-(1-(4-(2-(2-aminopyridin- | ++ |
| | 3-yl)-5-morpholino-3H-imidazo[4,5- | |
| | b]pyridin-3-yl)benzyl)piperidin- | |
| | 4-yl)-4-formyl-3-hydroxybenzamide | |
| 54 | 4-(4-(4-(2-(2-aminopyridin-3-yl)-6-phenyl-3H- | ++ |
| | imidazo[4,5-b]pyridin-3-yl)benzyl)piperazin- | |
| | 1-yl)-2-hydroxybenzaldehyde | |
| 55 | N-(1-(4-(2-(2-aminopyridin-3-yl)-5-phenyl-3H- | +++ |
| | imidazo[4,5-b]pyridin-3-yl)benzyl)piperidin- | |
| | 4-yl)-3-formyl-4-hydroxy-N-methylbenzamide | |
| 56 | 4-((1R,4R)-5-(4-(2-(2-aminopyridin-3-yl)-5- | ++ |
| | phenyl-3H-imidazo[4,5-b]pyridin-3-yl)benzyl)- | |
| | 2,5-diazabicyclo[2.2.1]heptane-2-carbonyl)-2- | |
| | hydroxybenzaldehyde | |
| 57 | 4-((1-(4-(2-(2-aminopyridin-3-yl)-5-phenyl-3H- | ++ |
| | imidazo[4,5-b]pyridin-3-yl)benzyl)piperidin-4- | |
| | yl)(methyl)amino)-2-hydroxybenzaldehyde | |
| 58 | (R)-N-(1-(4-(2-(2-aminopyridin- | +++ |
| | 3-yl)-5-phenyl-3H-imidazo[4,5- | |
| | b]pyridin-3-yl)benzyl)piperidin-3-yl)- | |
| | 3-formyl-4-hydroxybenzamide | |
| 59 | (R)-N-(1-(4-(2-(2-aminopyridin-3- | ++++ |
| | yl)-5-phenyl-3H-imidazo[4,5- | |
| | b]pyridin-3-yl)benzyl)pyrrolidin-3-yl)- | |
| | 3-formyl-4-hydroxybenzamide | |
| 60 | N-(1-(4-(2-(2-aminopyridin-3-yl)-5-phenyl-3H- | ++ |
| | imidazo[4,5-b]pyridin-3-yl)benzyl)piperidin- | |
| | 4-yl)-4-formyl-3-hydroxy-N-methylbenzamide | |
| 61 | N-(1-(4-(2-(2-aminopyridin-3-yl)-5-phenyl-3H- | +++ |
| | imidazo[4,5-b]pyridin-3-yl)benzyl)azetidin- | |
| | 3-yl)-3-formyl-4-hydroxybenzamide | |
| 62 | 5-(6-(4-(2-(2-aminopyridin-3- | ++++ |
| | yl)-5-phenyl-3H-imidazo[4,5-b]pyridin- | |
| | 3-yl)benzyl)-2,6-diazaspiro[3.3]heptane- | |
| | 2-carbonyl)-2-hydroxybenzaldehyde | |
| 63 | (R)-N-(1-(4-(2-(2-aminopyridin- | ++ |
| | 3-yl)-5-phenyl-3H-imidazo[4,5- | |
| | b]pyridin-3-yl)benzyl)piperidin- | |
| | 3-yl)-4-formyl-3-hydroxybenzamide | |
| 64 | (R)-N-(1-(4-(2-(2-aminopyridin- | ++ |
| | 3-yl)-5-phenyl-3H-imidazo[4,5- | |
| | b]pyridin-3-yl)benzyl)pyrrolidin-3-yl)- | |
| | 4-formyl-3-hydroxybenzamide | |
| 65 | N-(1-(4-(2-(2-aminopyridin-3- | +++ |
| | yl)-5-phenyl-3H-imidazo[4,5-b]pyridin- | |
| | 3-yl)benzyl)azetidin-3-yl)-4-formyl-3- | |
| | hydroxybenzamide | |
| 66 | 4-(6-(4-(2-(2-aminopyridin-3- | ++ |
| | yl)-5-phenyl-3H-imidazo[4,5-b]pyridin- | |
| | 3-yl)benzyl)-2,6-diazaspiro[3.3]heptane- | |
| | 2-carbonyl)-2-hydroxybenzaldehyde | |
| 67 | 5-(7-(4-(2-(2-aminopyridin-3- | ++ |
| | yl)-5-phenyl-3H-imidazo[4,5-b]pyridin- | |
| | 3-yl)benzyl)-2,7-diazaspiro[3.5]nonane- | |
| | 2-carbonyl)-2-hydroxybenzaldehyde | |
| 68 | N-(7-(4-(2-(2-aminopyridin-3- | +++ |
| | yl)-5-phenyl-3H-imidazo[4,5-b]pyridin- | |
| | 3-yl)benzyl)-7-azaspiro[3.5]nonan-2- | |
| | yl)-3-formyl-4-hydroxybenzamide | |
| 69 | N-(2-(4-(2-(2-aminopyridin-3- | ++++ |
| | yl)-5-phenyl-3H-imidazo[4,5-b]pyridin- | |
| | 3-yl)benzyl)-2-azaspiro[3.3]heptan-6-yl)- | |
| | 3-formyl-4-hydroxybenzamide | |
| 70 | N-(2-(4-(2-(2-aminopyridin-3-yl)-5- | ++ |
| | phenyl-3H-imidazo[4,5-b]pyridin- | |
| | 3-yl)benzyl)-2-azaspiro[3.3]heptan-6-yl)- | |
| | 4-formyl-3-hydroxybenzamide | |
| 71 | 4-(7-(4-(2-(2-aminopyridin-3- | + |
| | yl)-5-phenyl-3H-imidazo[4,5-b]pyridin- | |
| | 3-yl)benzyl)-2,7-diazaspiro[3.5]nonane- | |
| | 2-carbonyl)-2-hydroxybenzaldehyde | |
| 72 | 4-(4-(4-(2-(2-aminopyridin-3-yl)- | +++ |
| | 5-phenyl-3H-imidazo[4,5-b]pyridin- | |
| | 3-yl)benzyl)-1,4-diazepane-1-carbonyl)-2- | |
| | hydroxybenzaldehyde | |
| 73 | 4-(7-(4-(2-(2-aminopyridin-3-yl)- | ++ |
| | 5-phenyl-3H-imidazo[4,5-b]pyridin-3- | |
| | yl)benzyl)-2,7-diazaspiro[3.5]nonan-2-yl)-2- | |
| | hydroxybenzaldehyde | |
| 74 | 5-(8-(4-(2-(2-aminopyridin-3-yl)- | +++ |
| | 5-phenyl-3H-imidazo[4,5-b]pyridin- | |
| | 3-yl)benzyl)-2,8-diazaspiro[4.5]decane- | |
| | 2-carbonyl)-2-hydroxybenzaldehyde | |
| 75 | 4-(8-(4-(2-(2-aminopyridin-3-yl)- | ++ |
| | 5-phenyl-3H-imidazo[4,5-b]pyridin- | |
| | 3-yl)benzyl)-2,8-diazaspiro[4.5]decane- | |
| | 2-carbonyl)-2-hydroxybenzaldehyde | |
| 76 | 4-(8-(4-(2-(2-aminopyridin-3-yl)- | ++ |
| | 5-phenyl-3H-imidazo[4,5- | |
| | b]pyridin-3-yl)benzyl)-2,8- | |
| | diazaspiro[4.5]decan-2-yl)-2- | |
| | hydroxybenzaldehyde | |
| 77 | 5-(9-(4-(2-(2-aminopyridin-3- | ++ |
| | yl)-5-phenyl-3H-imidazo[4,5-b]pyridin- | |
| | 3-yl)benzyl)-3,9-diazaspiro[5.5]undecane-3- | |
| | carbonyl)-2-hydroxybenzaldehyde | |
| 78 | 4-(9-(4-(2-(2-aminopyridin-3- | ++ |
| | yl)-5-phenyl-3H-imidazo[4,5-b]pyridin-3- | |
| | yl)benzyl)-3,9-diazaspiro[5.5]undecan-3-yl)-2- | |
| | hydroxybenzaldehyde | |
| 79 | 4-(6-(4-(2-(2-aminopyridin-3-yl)-5- | +++ |
| | phenyl-3H-imidazo[4,5-b]pyridin-3- | |
| | yl)benzyl)-2,6-diazaspiro[3.4]octan-2-yl)-2- | |
| | hydroxybenzaldehyde | |
| 80 | 5-(7-(4-(2-(2-aminopyridin-3-yl)-5- | ++ |
| | phenyl-3H-imidazo[4,5-b]pyridin-3- | |
| | yl)benzyl)-2,7-diazaspiro[4.4]nonane- | |
| | 2-carbonyl)-2-hydroxybenzaldehyde | |
| 81 | 4-(7-(4-(2-(2-aminopyridin-3-yl)- | ++ |
| | 5-phenyl-3H-imidazo[4,5-b]pyridin-3- | |
| | yl)benzyl)-2,7-diazaspiro[4.4]nonane- | |
| | 2-carbonyl)-2-hydroxybenzaldehyde | |
| 82 | 4-(7-(4-(2-(2-aminopyridin-3-yl)- | ++ |
| | 5-phenyl-3H-imidazo[4,5- | |
| | b]pyridin-3-yl)benzyl)-2,7- | |
| | diazaspiro[4.4]nonan-2-yl)-2- | |
| | hydroxybenzaldehyde | |
| 83 | 5-(7-(4-(2-(2-aminopyridin-3-yl)- | ++ |
| | 5-phenyl-3H-imidazo[4,5- | |
| | b]pyridin-3-yl)benzyl)-2, 7- | |
| | diazaspiro[4.4]nonan-2-yl)-2- | |
| | hydroxybenzaldehyde | |
| 84 | 5-(2-(4-(2-(2-aminopyridin-3- | ++ |
| | yl)-5-phenyl-3H-imidazo[4,5-b]pyridin- | |
| | 3-yl)benzyl)-2,8-diazaspiro[4.5]decane- | |
| | 8-carbonyl)-2-hydroxybenzaldehyde | |
| 85 | 4-(2-(4-(2-(2-aminopyridin-3-yl)- | ++ |
| | 5-phenyl-3H-imidazo[4,5- | |
| | b]pyridin-3-yl)benzyl)-2,8- | |
| | diazaspiro[4.5]decan-8-yl)-2- | |
| | hydroxybenzaldehyde | |
| 86 | 5-(2-(4-(2-(2-aminopyridin-3-yl)- | ++ |
| | 5-phenyl-3H-imidazo[4,5-b]pyridin- | |
| | 3-yl)benzyl)-2,7-diazaspiro[4.5]decane- | |
| | 7-carbonyl)-2-hydroxybenzaldehyde | |
| 87 | 4-(2-(4-(2-(2-aminopyridin-3-yl)- | ++ |
| | 5-phenyl-3H-imidazo[4,5-b]pyridin- | |
| | 3-yl)benzyl)-2,7-diazaspiro[4.5]decane- | |
| | 7-carbonyl)-2-hydroxybenzaldehyde | |
| 88 | 4-(2-(4-(2-(2-aminopyridin-3-yl)- | ++ |
| | 5-phenyl-3H-imidazo[4,5-b]pyridin- | |
| | 3-yl)benzyl)-2,7-diazaspiro[4.5]decan-7-yl)-2- | |
| | hydroxybenzaldehyde | |
| 89 | 4-(6-(4-(2-(2-aminopyridin-3-yl)- | +++ |
| | 5-phenyl-3H-imidazo[4,5-b]pyridin-3- | |
| | yl)benzyl)-2,6-diazaspiro[3.3]heptan-2-yl)-2- | |
| | hydroxybenzaldehyde | |
| 90 | 4-(2-(4-(2-(2-aminopyridin-3-yl)-5-phenyl- | ++ |
| | 3H-imidazo[4,5-b]pyridin-3- | |
| | yl)benzyl)-2,8-diazaspiro[4.5]decane- | |
| | 8-carbonyl)-2-hydroxybenzaldehyde | |
| 91 | 5-(6-(4-(2-(2-aminopyridin-3-yl)-5- | ++ |
| | phenyl-3H-imidazo[4,5-b]pyridin-3- | |
| | yl)benzyl)-2,6-diazaspiro[3.3]heptan-2-yl)-2- | |
| | hydroxybenzaldehyde | |
| 92 | 5-(2-(4-(2-(2-aminopyridin-3-yl)- | +++ |
| | 5-phenyl-3H-imidazo[4,5-b]pyridin- | |
| | 3-yl)benzyl)-2,6-diazaspiro[3.4]octane-6- | |
| | carbonyl)-2-hydroxybenzaldehyde | |
| 93 | 4-(2-(4-(2-(2-aminopyridin-3-yl)- | ++ |
| | 5-phenyl-3H-imidazo[4,5-b]pyridin-3- | |
| | yl)benzyl)-2,6-diazaspiro[3.4]octane- | |
| | 6-carbonyl)-2-hydroxybenzaldehyde | |
| 94 | 4-(2-(4-(2-(2-aminopyridin-3-yl)- | +++ |
| | 5-phenyl-3H-imidazo[4,5-b]pyridin-3- | |
| | yl)benzyl)-2,6-diazaspiro[3.4]octan-6-yl)-2- | |
| | hydroxybenzaldehyde | |
| 95 | 5-(2-(4-(2-(2-aminopyridin-3-yl)- | ++ |
| | 5-phenyl-3H-imidazo[4,5-b]pyridin- | |
| | 3-yl)benzyl)-2,7-diazaspiro[3.5]nonane- | |
| | 7-carbonyl)-2-hydroxybenzaldehyde | |
| 96 | 4-(2-(4-(2-(2-aminopyridin-3-yl)-5-phenyl- | ++ |
| | 3H-imidazo[4,5-b]pyridin-3-yl)benzyl)- | |
| | 2,7-diazaspiro[3.5]nonane-7-carbonyl)- | |
| | 2-hydroxybenzaldehyde | |
| 97 | 4-(2-(4-(2-(2-aminopyridin-3-yl)- | ++ |
| | 5-phenyl-3H-imidazo[4,5-b]pyridin-3- | |
| | yl)benzyl)-2,7-diazaspiro[3.5]nonan-7-yl)-2- | |
| | hydroxybenzaldehyde | |
| 98 | (R)-5-(7-(4-(2-(2-aminopyridin-3-yl)-5-phenyl- | +++ |
| | 3H-imidazo[4,5-b]pyridin-3-yl)benzyl)- | |
| | 2,7-diazaspiro[4.4]nonane- | |
| | 2-carbonyl)-2-hydroxybenzaldehyde | |
| 99 | (R)-4-(7-(4-(2-(2-aminopyridin-3-yl)-5-phenyl- | ++ |
| | 3H-imidazo[4,5-b]pyridin-3-yl)benzyl)- | |
| | 2, 7-diazaspiro[4.4]nonane- | |
| | 2-carbonyl)-2-hydroxybenzaldehyde | |
| 100 | (R)-4-(7-(4-(2-(2-aminopyridin-3-yl)-5-phenyl- | ++ |
| | 3H-imidazo[4,5-b]pyridin-3-yl)benzyl)-2,7- | |
| | diazaspiro[4.4]nonan-2-yl)- | |
| | 2-hydroxybenzaldehyde | |
| 101 | (S)-5-(7-(4-(2-(2-aminopyridin-3-yl)-5- | ++ |
| | phenyl-3H-imidazo[4,5-b]pyridin-3- | |
| | yl)benzyl)-2,7-diazaspiro[4.4]nonane- | |
| | 2-carbonyl)-2-hydroxybenzaldehyde | |
| 102 | (S)-4-(7-(4-(2-(2-aminopyridin-3-yl)-5- | ++ |
| | phenyl-3H-imidazo[4,5-b]pyridin-3-yl)benzyl)- | |
| | 2,7-diazaspiro[4.4]nonane-2-carbonyl)- | |
| | 2-hydroxybenzaldehyde | |
| 103 | (S)-4-(7-(4-(2-(2-aminopyridin-3-yl)-5- | ++ |
| | phenyl-3H-imidazo[4,5-b]pyridin-3-yl)benzyl)- | |
| | 2,7-diazaspiro[4.4]nonan-2-yl)-2- | |
| | hydroxybenzaldehyde | |
| 104 | (R)-4-(4-(4-(2-(2-aminopyridin-3-yl)-5-phenyl- | ++ |
| | 3H-imidazo[4,5-b]pyridin-3-yl)benzyl)-2- | |
| | methylpiperazin-1-yl)-2-hydroxybenzaldehyde | |
| 105 | (S)-5-(4-(4-(2-(2-aminopyridin-3-yl)-5-phenyl- | ++ |
| | 3H-imidazo[4,5-b]pyridin-3- | |
| | yl)benzyl)-2-methylpiperazine- | |
| | 1-carbonyl)-2-hydroxybenzaldehyde | |
| 106 | (S)-4-(4-(4-(2-(2-aminopyridin-3-yl)-5- | + |
| | phenyl-3H-imidazo[4,5-b]pyridin-3- | |
| | yl)benzyl)-2-methylpiperazine- | |
| | 1-carbonyl)-2-hydroxybenzaldehyde | |
| 107 | (S)-4-(4-(4-(2-(2-aminopyridin-3-yl)-5- | ++ |
| | phenyl-3H-imidazo[4,5-b]pyridin-3- | |
| | yl)benzyl)-2-methylpiperazin- | |
| | 1-yl)-2-hydroxybenzaldehyde | |
| 108 | 4-(7-(4-(2-(2-aminopyridin-3-yl)-5- | ++ |
| | phenyl-3H-imidazo[4,5-b]pyridin-3- | |
| | yl)benzyl)-4,7-diazaspiro[2.5]octan-4-yl)-2- | |
| | hydroxybenzaldehyde | |
| 109 | 4-(3-(4-(2-(2-aminopyridin-3-yl)-5- | + |
| | phenyl-3H-imidazo[4,5-b]pyridin- | |
| | 3-yl)benzyl)-3,8-diazabicyclo[3.2.1]octane-8- | |
| | carbonyl)-2-hydroxybenzaldehyde | |
| 110 | 5-(3-(4-(2-(2-aminopyridin-3-yl)-5- | + |
| | phenyl-3H-imidazo[4,5-b]pyridin-3- | |
| | yl)benzyl)-3,8-diazabicyclo[3.2. 1 ]octane-8- | |
| | carbonyl)-2-hydroxybenzaldehyde | |
| 111 | 4-(3-(4-(2-(2-aminopyridin-3-yl)-5- | + |
| | phenyl-3H-imidazo[4,5-b]pyridin-3-yl)benzyl)- | |
| | 3,8-diazabicyclo[3.2.1]octan-8-yl)-2- | |
| | hydroxybenzaldehyde | |
| 112 | 5-(8-(4-(2-(2-aminopyridin-3-yl)-5- | + |
| | phenyl-3H-imidazo[4,5-b]pyridin- | |
| | 3-yl)benzyl)-3,8-diazabicyclo[3.2.1 ]octane-3- | |
| | carbonyl)-2-hydroxybenzaldehyde | |
| 113 | 4-(8-(4-(2-(2-aminopyridin-3-yl)-5- | ++ |
| | phenyl-3H-imidazo[4,5-b]pyridin-3- | |
| | yl)benzyl)-3,8-diazabicyclo[3.2.1 ]octane-3- | |
| | carbonyl)-2-hydroxybenzaldehyde | |
| 114 | 4-(8-(4-(2-(2-aminopyridin-3-yl)-5- | ++ |
| | phenyl-3H-imidazo[4,5-b]pyridin-3- | |
| | yl)benzyl)-3,8-diazabicyclo[3.2.1]octan- | |
| | 3-yl)-2-hydroxybenzaldehyde | |
| 115 | N-(1-(4-(2-(2-aminopyridin-3-yl)-5- | +++ |
| | phenyl-3H-imidazo[4,5-b]pyridin-3- | |
| | yl)benzyl)azepan-4-yl)-3-formyl-4- | |
| | hydroxybenzamide | |
| 116 | N-(1-(4-(2-(2-aminopyridin-3-yl)-5- | ++ |
| | phenyl-3H-imidazo[4,5-b]pyridin-3- | |
| | yl)benzyl)azepan-4-yl)-4-formyl-3- | |
| | hydroxybenzamide | |
| 117 | N-(1-(4-(2-(2-aminopyridin-3-yl)-5- | + |
| | phenyl-3H-imidazo[4,5-b]pyridin-3- | |
| | yl)benzyl)azepan-3-yl)-3-formyl-4- | |
| | hydroxybenzamide | |
| 118 | N-(1-(4-(2-(2-aminopyridin-3-yl)-5- | + |
| | phenyl-3H-imidazo[4,5-b]pyridin-3- | |
| | yl)benzyl)azepan-3-yl)-4-formyl-3- | |
| | hydroxybenzamide | |
| 119 | 5-(4-(4-(2-(2-aminopyridin-3-yl)-5- | +++ |
| | phenyl-3H-imidazo[4,5-b]pyridin-3- | |
| | yl)benzyl)-1,4-diazepane-1-carbonyl)-2- | |
| | hydroxybenzaldehyde | |
| 120 | 4-(4-(4-(2-(2-aminopyridin-3-yl)-5-phenyl- | +++ |
| | 3H-imidazo[4,5-b]pyridin-3-yl)benzyl)-1,4- | |
| | diazepan-1-yl)-2-hydroxybenzaldehyde | |
| 121 | 5-((1S,4S)-5-(4-(2-(2-aminopyridin-3-yl)-5- | ++ |
| | phenyl-3H-imidazo[4,5-b]pyridin-3-yl)benzyl)- | |
| | 2,5-diazabicyclo[2.2.1]heptane-2-carbonyl)-2- | |
| | hydroxybenzaldehyde | |
| 122 | 4-((1S,4S)-5-(4-(2-(2-aminopyridin-3-yl)-5- | +++ |
| | phenyl-3H-imidazo[4,5-b]pyridin-3-yl)benzyl)- | |
| | 2,5-diazabicyclo[2.2.1]heptane-2-carbonyl)-2- | |
| | hydroxybenzaldehyde | |
| 123 | 4-((1S,4S)-5-(4-(2-(2-aminopyridin-3-yl)-5- | ++ |
| | phenyl-3H-imidazo[4,5-b]pyridin-3-yl)benzyl)- | |
| | 2,5-diazabicyclo[2.2.1]heptan-2-yl)- | |
| | 2-hydroxybenzaldehyde | |
| 124 | 4-((1R,4R)-5-(4-(2-(2-aminopyridin-3-yl)-5- | +++ |
| | phenyl-3H-imidazo[4,5-b]pyridin-3-yl)benzyl)- | |
| | 2,5-diazabicyclo[2.2.1]heptan-2-yl)- | |
| | 2-hydroxybenzaldehyde | |
| 125 | 4-(5-(4-(2-(2-aminopyridin-3-yl)-5- | ++ |
| | phenyl-3H-imidazo[4,5-b]pyridin-3-yl)benzyl)- | |
| | 2,5-diazabicyclo[2.2.2]octane-2- | |
| | carbonyl)-2-hydroxybenzaldehyde | |
| 126 | 5-(5-(4-(2-(2-aminopyridin-3-yl)-5-phenyl- | ++ |
| | 3H-imidazo[4,5-b]pyridin-3-yl)benzyl)-2,5- | |
| | diazabicyclo[2.2.2]octane-2- | |
| | carbonyl)-2-hydroxybenzaldehyde | |
| 127 | 4-(5-(4-(2-(2-aminopyridin-3-yl)-5-phenyl- | ++ |
| | 3H-imidazo[4,5-b]pyridin-3-yl)benzyl)-2,5- | |
| | diazabicyclo[2.2.2]octan-2-yl)-2- | |
| | hydroxybenzaldehyde | |
| 128 | 2-((1-(4-(2-(2-aminopyridin-3-yl)-5-phenyl-3H- | ++ |
| | imidazo[4,5-b]pyridin-3-yl)benzyl)piperidin- | |
| | 4-yl)oxy)-6-hydroxybenzaldehyde | |
| 129 | 4-((1-(4-(2-(2-aminopyridin-3-yl)-5-phenyl-3H- | +++ |
| | imidazo[4,5-b]pyridin-3-yl)benzyl)piperidin- | |
| | 4-yl)oxy)-2-hydroxybenzaldehyde | |
| 130 | 5-((1-(4-(2-(2-aminopyridin-3-yl)-5-phenyl-3H- | ++ |
| | imidazo[4,5-b]pyridin-3-yl)benzyl)piperidin- | |
| | 4-yl)oxy)-2-hydroxybenzaldehyde | |
| 131 | 5-((3aR,6aS)-5-(4-(2-(2-aminopyridin- | ++ |
| | 3-yl)-5-phenyl-3H-imidazo[4,5-b]pyridin- | |
| | 3-yl)benzyl)octahydropyrrolo[3,4- | |
| | c]pyrrole-2-carbonyl)-2-hydroxybenzaldehyde | |
| 132 | 4-((3aR,6aS)-5-(4-(2-(2-aminopyridin- | ++ |
| | 3-yl)-5-phenyl-3H-imidazo[4,5-b]pyridin-3- | |
| | yl)benzyl)octahydropyrrolo[3,4- | |
| | c]pyrrole-2-carbonyl)-2-hydroxybenzaldehyde | |
| 133 | 4-((3aR,6aS)-5-(4-(2-(2-aminopyridin- | ++ |
| | 3-yl)-5-phenyl-3H-imidazo[4,5-b]pyridin- | |
| | 3-yl)benzyl)hexahydropyrrolo[3,4- | |
| | c]pyrrol-2(1H)-yl)-2-hydroxybenzaldehyde | |
| 134 | 5-((3aR,6aS)-5-(4-(2-(2-aminopyridin- | ++ |
| | 3-yl)-5-phenyl-3H-imidazo[4,5-b]pyridin- | |
| | 3-yl)benzyl)hexahydropyrrolo[3,4- | |
| | c]pyrrol-2(1H)-yl)-2-hydroxybenzaldehyde | |
| 135 | 5-((3aS,6aS)-5-(4-(2-(2-aminopyridin- | +++ |
| | 3-yl)-5-phenyl-3H-imidazo[4,5-b]pyridin- | |
| | 3-yl)benzyl)octahydropyrrolo[3,4- | |
| | c]pyrrole-2-carbonyl)-2-hydroxybenzaldehyde | |
| 136 | 4-((3aS,6aS)-5-(4-(2-(2-aminopyridin- | ++ |
| | 3-yl)-5-phenyl-3H-imidazo[4,5-b]pyridin- | |
| | 3-yl)benzyl)octahydropyrrolo[3,4- | |
| | c]pyrrole-2-carbonyl)-2-hydroxybenzaldehyde | |
| 137 | 2-((4-(2-(2-aminopyridin-3-yl)-5-phenyl- | + |
| | 3H-imidazo[4,5-b]pyridin-3-yl)benzyl)oxy)- | |
| | 6-hydroxybenzaldehyde | |
| 138 | 4-((4-(2-(2-aminopyridin-3-yl)-5-phenyl- | ++ |
| | 3H-imidazo[4,5-b]pyridin-3-yl)benzyl)oxy)- | |
| | 2-hydroxybenzaldehyde | |
| 139 | 5-((4-(2-(2-aminopyridin-3-yl)-5-phenyl- | + |
| | 3H-imidazo[4,5-b]pyridin-3-yl)benzyl)oxy)- | |
| | 2-hydroxybenzaldehyde | |
| 140 | (S)-N-(1-(4-(2-(2-aminopyridin-3-yl)- | +++ |
| | 5-phenyl-3H-imidazo[4,5-b]pyridin-3- | |
| | yl)phenyl)ethyl)-3-formyl-4- | |
| | hydroxybenzamide | |
| 141 | 5-(4-(4-(2-(2-aminopyridin-3-yl)-5-(2- | +++ |
| | fluorophenyl)-3H-imidazo[4,5- | |
| | b]pyridin-3-yl)benzyl)piperazine-1- | |
| | carbonyl)-2-hydroxybenzaldehyde | |
| 142 | 4-(4-(4-(2-(2-aminopyridin-3- | + |
| | yl)-5-(2-fluorophenyl)-3H-imidazo[4,5- | |
| | b]pyridin-3-yl)benzyl)piperazine-1- | |
| | carbonyl)-2-hydroxybenzaldehyde | |
| 143 | 5-(4-(4-(2-(2-aminopyridin- | ++ |
| | 3-yl)-5-(4-fluorophenyl)-3H- | |
| | imidazo[4,5-b]pyridin-3-yl)benzyl)piperazine- | |
| | 1-carbonyl)-2-hydroxybenzaldehyde | |
| 144 | 4-(4-(4-(2-(2-aminopyridin- | ++ |
| | 3-yl)-5-(4-fluorophenyl)-3H- | |
| | imidazo[4,5-b]pyridin-3-yl)benzyl)piperazine- | |
| | 1-carbonyl)-2-hydroxybenzaldehyde | |
| 145 | 4-(4-(4-(2-(2-aminopyridin-3- | ++ |
| | yl)-5-(2-chlorophenyl)-3H-imidazo[4,5- | |
| | b]pyridin-3-yl)benzyl)piperazin-1-yl)-2- | |
| | hydroxybenzaldehyde | |
| 146 | 4-(4-(4-(2-(2-aminopyridin- | + |
| | 3-yl)-5-(4-chlorophenyl)-3H-imidazo[4,5- | |
| | b]pyridin-3-yl)benzyl)piperazin-1-yl)-2- | |
| | hydroxybenzaldehyde | |
| 147 | 5-(4-(4-(2-(2-aminopyridin- | + |
| | 3-yl)-5-(4-chlorophenyl)-3H- | |
| | imidazo[4,5-b]pyridin-3-yl)benzyl)piperazine- | |
| | 1-carbonyl)-2-hydroxybenzaldehyde | |
| 148 | 4-(4-(4-(2-(2-aminopyridin- | + |
| | 3-yl)-5-(4-chlorophenyl)-3H- | |
| | imidazo[4,5-b]pyridin-3-yl)benzyl)piperazine- | |
| | 1-carbonyl)-2-hydroxybenzaldehyde | |
| 149 | 5-(4-(4-(2-(2-aminopyridin-3- | + |
| | yl)-5-(4-chlorophenyl)-3H-imidazo[4,5- | |
| | b]pyridin-3-yl)benzyl)piperazin-1-yl)-2- | |
| | hydroxybenzaldehyde | |
| 150 | 5-(4-(4-(2-(2-aminopyridin-3-yl)- | ++ |
| | 5-(cyclohex-1-en-1-yl)-3H-imidazo[4,5- | |
| | b]pyridin-3-yl)benzyl)piperazine- | |
| | 1-carbonyl)-2-hydroxybenzaldehyde | |
| 151 | 4-(4-(4-(2-(2-aminopyridin-3-yl)- | + |
| | 5-(cyclohex-1-en-1-yl)-3H-imidazo[4,5- | |
| | b]pyridin-3-yl)benzyl)piperazine- | |
| | 1-carbonyl)-2-hydroxybenzaldehyde | |
| 152 | 4-(9-(4-(2-(2-aminopyridin-3-yl)-5- | ++ |
| | phenyl-3H-imidazo[4,5-b]pyridin-3- | |
| | yl)benzyl)-3,9-diazaspiro[5.5]undecane-3- | |
| | carbonyl)-2-hydroxybenzaldehyde | |
| 153 | 4-(6-(4-(2-(2-aminopyridin-3-yl)-5- | ++ |
| | phenyl-3H-imidazo[4,5-b]pyridin-3- | |
| | yl)benzyl)-2,6-diazaspiro[3.4]octane- | |
| | 2-carbonyl)-2-hydroxybenzaldehyde | |

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1-53. (canceled)

54. A compound having the structure of Formula (Ia), or a pharmaceutically acceptable salt or solvate thereof: 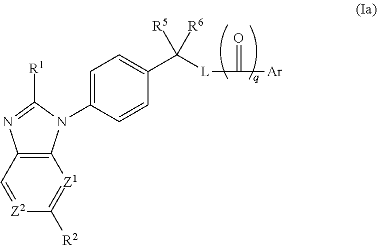
wherein:
Z¹ is N, C—H, or C—R;
Z² is N, C—H, or C—R⁴;
Ar is selected from 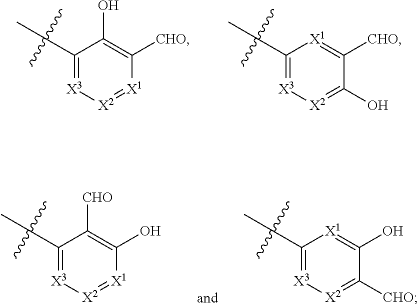
X¹ is N or C—R⁷;
X² is N or C—R⁷;
X³ is N or C—R⁷;
R¹ is optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, halogen, —OH, —SH, optionally substituted C1-C6 alkoxy, —S-(optionally substituted C1-C6 alkyl), —CN, optionally substituted C1-C6 alkyl, and optionally substituted aryl;
L is selected from —N(R⁸)—, or a divalent radical selected from: 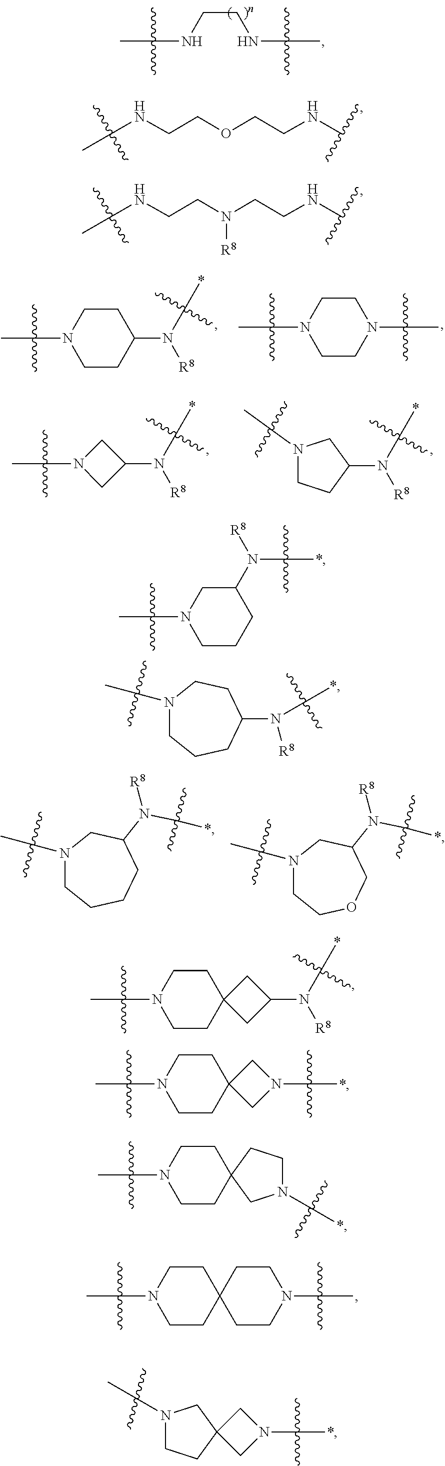 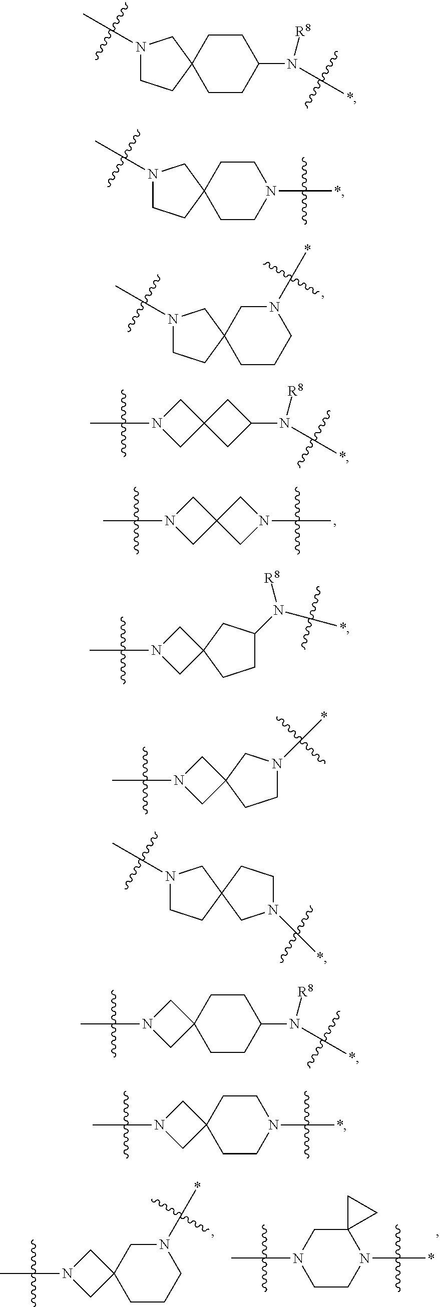 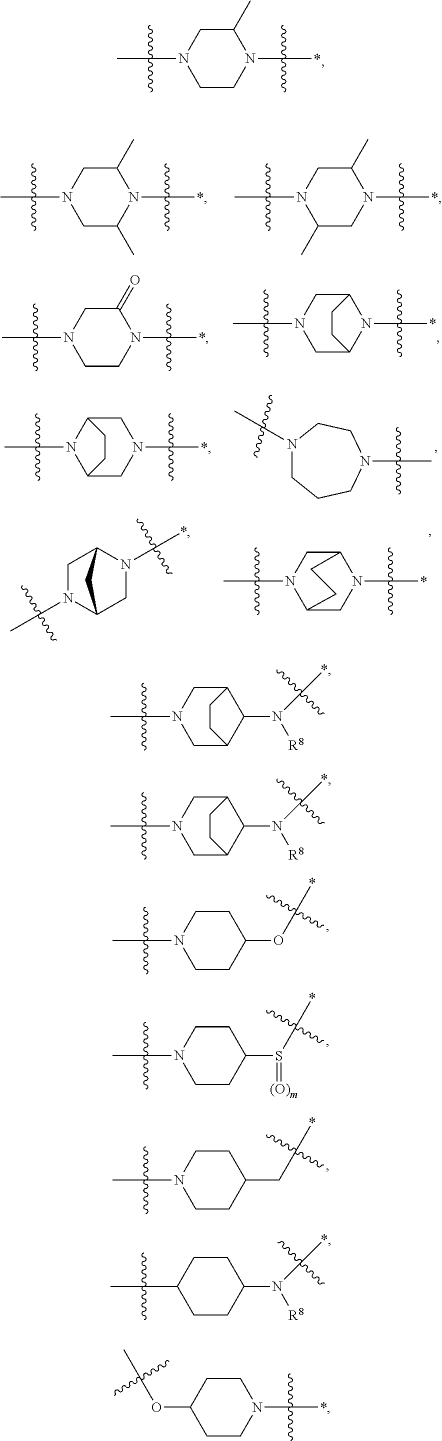 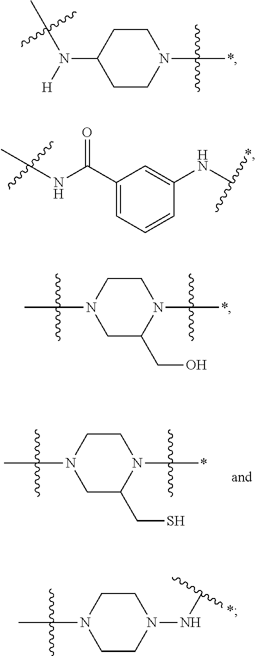
wherein the asterisk (*) indicates the bond to the —CO—Ar group;
R⁸ is hydrogen, optionally substituted C1-C6 alkyl, optionally substituted C3-C7 cycloalkyl, or optionally substituted heterocyclyl;
m is 0, 1, or 2;
n is 1, 2, or 3;
q is O or 1; and
wherein the compound of Formula (Ia) is not

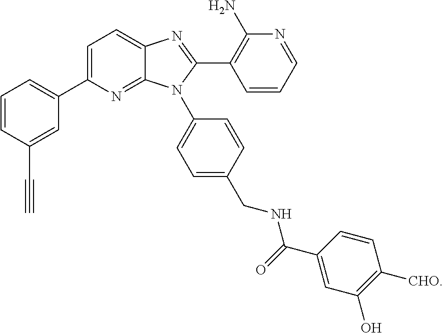 [SMILES: C#Cc1cccc(-c2ccc3nc(-c4cccnc4N)n(-c4ccc(CNC(=O)c5ccc(C=O)c(O)c5)cc4)c3n2)c1]

55. The compound of claim 54, or pharmaceutically acceptable salt or solvate thereof, wherein Z¹ is N.

56. The compound of claim 54, or pharmaceutically acceptable salt or solvate thereof, wherein Z² is C—H.

57. The compound of claim 54, or pharmaceutically acceptable salt or solvate thereof, wherein R¹ is an optionally substituted pyridyl.

58. The compound of claim 54, or pharmaceutically acceptable salt or solvate thereof, wherein R² is optionally substituted aryl.

59. The compound of claim 58, or pharmaceutically acceptable salt or solvate thereof, wherein the optionally substituted aryl is an optionally substituted phenyl.

60. The compound of claim 54, or pharmaceutically acceptable salt or solvate thereof, wherein L is —N(R⁸)—.

61. The compound of claim 54, or pharmaceutically acceptable salt or solvate thereof, wherein L is selected from:

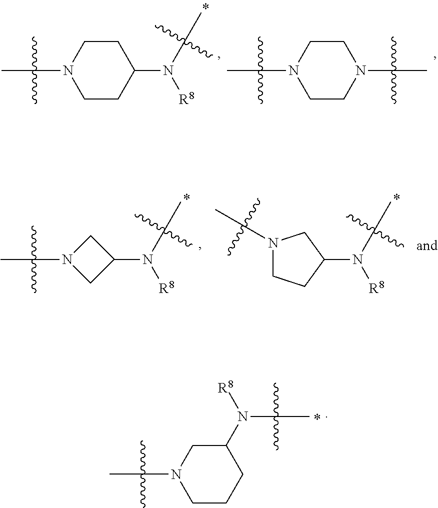

62. The compound of claim 54, or pharmaceutically acceptable salt or solvate thereof, wherein L is selected from:

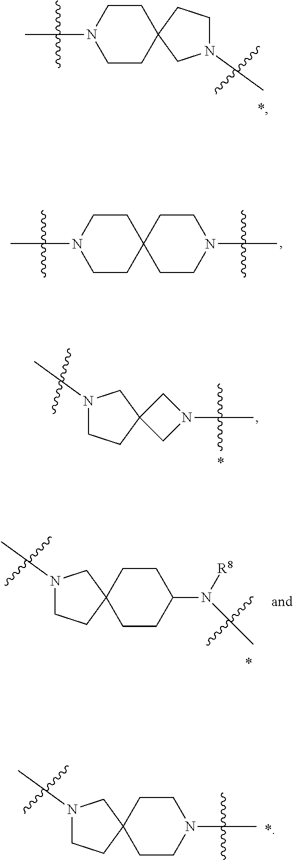

63. The compound of claim 54, or pharmaceutically acceptable salt or solvate thereof, wherein L is selected from:

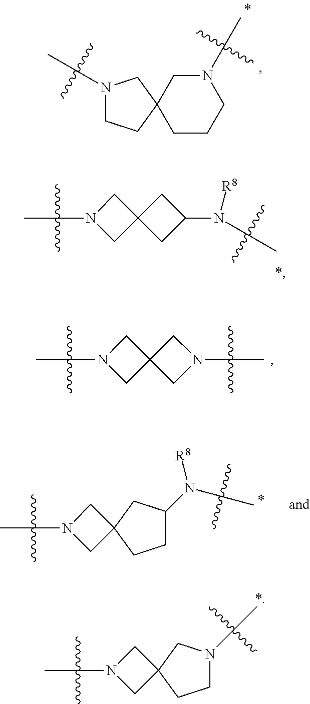

64. The compound of claim 54, or pharmaceutically acceptable salt or solvate thereof, wherein L is selected from:

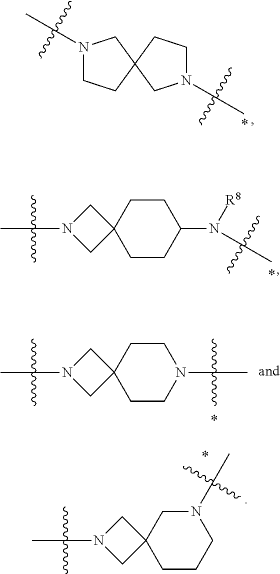

65. The compound of claim 54, or pharmaceutically acceptable salt or solvate thereof, wherein R⁸ is hydrogen or optionally substituted C1-C6 alkyl.

66. The compound of claim 54, or pharmaceutically acceptable salt or solvate thereof, wherein q is 0.

67. The compound of claim 54, or pharmaceutically acceptable salt or solvate thereof, wherein q is 1.

68. The compound of claim 54, or pharmaceutically acceptable salt or solvate thereof, wherein Ar is

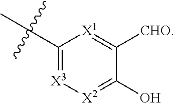

69. The compound of claim 54, or pharmaceutically acceptable salt or solvate thereof, wherein Ar is

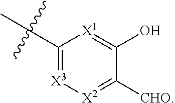

70. The compound of claim 68, or pharmaceutically acceptable salt or solvate thereof, wherein X¹ is C—H; X² is C—H; and X³ is C—H.

71. The compound of claim 69, or pharmaceutically acceptable salt or solvate thereof, wherein X¹ is C—H; X² is C—H; and X³ is C—H.

72. The compound of claim 54, or pharmaceutically acceptable salt or solvate thereof, selected from the group consisting of:
N-(4-(2-(2-aminopyridin-3-yl)-5-phenyl-3H-imidazo[4,5-b]pyridin-3-yl)benzyl)-4-formyl-3-hydroxybenzamide;
N-(4-(2-(2-aminopyridin-3-yl)-3H-imidazo[4,5-b]pyridin-3-yl)benzyl)-3-formyl-4-hydroxybenzamide;
N-(4-(2-(2-aminopyridin-3-yl)-3H-imidazo[4,5-b]pyridin-3-yl)benzyl)-4-formyl-3-hydroxybenzamide;
N-(4-(2-(2-aminopyridin-3-yl)-5-phenyl-3H-imidazo[4,5-b]pyridin-3-yl)benzyl)-3-formyl-4-hydroxybenzamide;
5-(4-(4-(2-(2-aminopyridin-3-yl)-5-phenyl-3H-imidazo[4,5-b]pyridin-3-yl)benzyl)piperazine-1-carbonyl)-2-hydroxybenzaldehyde;
4-(4-(4-(2-(2-aminopyridin-3-yl)-5-phenyl-3H-imidazo[4,5-b]pyridin-3-yl)benzyl)piperazine-1-carbonyl)-2-hydroxybenzaldehyde;
N-(1-(4-(2-(2-aminopyridin-3-yl)-5-phenyl-3H-imidazo[4,5-b]pyridin-3-yl)benzyl)piperidin-4-yl)-3-formyl-4-hydroxybenzamide;
N-(1-(4-(2-(2-aminopyridin-3-yl)-5-phenyl-3H-imidazo[4,5-b]pyridin-3-yl)benzyl)piperidin-4-yl)-4-formyl-3-hydroxybenzamide;
4-(4-(4-(2-(2-aminopyridin-3-yl)-5-phenyl-3H-imidazo[4,5-b]pyridin-3-yl)benzyl)piperazin-1-yl)-2-hydroxybenzaldehyde;
5-(4-(4-(2-(2-aminopyridin-3-yl)-5-phenyl-3H-imidazo[4,5-b]pyridin-3-yl)benzyl)piperazin-1-yl)-2-hydroxybenzaldehyde; and
N-(1-(4-(2-(2-aminopyridin-3-yl)-5-phenyl-3H-imidazo[4,5-b]pyridin-3-yl)phenyl)cyclobutyl)-3-formyl-4-hydroxybenzamide.

73. A compound, or pharmaceutically acceptable salt or solvate thereof, selected from the group consisting of:
N-(1-(4-(2-(2-aminopyridin-3-yl)-5-phenyl-3H-imidazo[4,5-b]pyridin-3-yl)benzyl)piperidin-4-yl)-N-cyclopropyl-3-formyl-4-hydroxybenzamide;
N-(1-(4-(2-(2-aminopyridin-3-yl)-5-phenyl-3H-imidazo[4,5-b]pyridin-3-yl)benzyl)piperidin-4-yl)-N-ethyl-3-formyl-4-hydroxybenzamide;
5-(4-(4-(2-(2-aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(2-oxo-1,2-dihydropyridin-3-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-3H-imidazo[4,5-b]pyridin-3-yl)benzyl)piperazin-1-yl)-2-hydroxybenzaldehyde;
4-((4-(2-(2-aminopyridin-3-yl)-5-phenyl-3H-imidazo[4,5-b]pyridin-3-yl)benzyl)(methyl)amino)-2-hydroxybenzaldehyde;
5-((4-(2-(2-aminopyridin-3-yl)-5-phenyl-3H-imidazo[4,5-b]pyridin-3-yl)benzyl)(methyl)amino)-2-hydroxybenzaldehyde;
N-(4-(2-(2-aminopyridin-3-yl)-5-phenyl-3H-imidazo[4,5-b]pyridin-3-yl)benzyl)-5-formyl-2-hydroxybenzamide;
N-(4-(2-(2-aminopyridin-3-yl)-5-phenyl-3H-imidazo[4,5-b]pyridin-3-yl)benzyl)-3-formyl-2-hydroxybenzamide;
N-(4-(2-(2-aminopyridin-3-yl)-5-phenyl-3H-imidazo[4,5-b]pyridin-3-yl)benzyl)-2-formyl-3-hydroxybenzamide;
2-amino-N-(4-(2-(2-aminopyridin-3-yl)-5-phenyl-3H-imidazo[4,5-b]pyridin-3-yl)benzyl)-5-formyl-4-hydroxybenzamide;
3-(4-(4-(2-(2-aminopyridin-3-yl)-5-phenyl-3H-imidazo[4,5-b]pyridin-3-yl)benzyl)piperazin-1-yl)-2-hydroxybenzaldehyde;
4-(4-(4-(2-(2-aminopyridin-3-yl)-5-phenyl-3H-imidazo[4,5-b]pyridin-3-yl)benzyl)piperazin-1-yl)-2-fluoro-6-hydroxybenzaldehyde;
4-(4-(4-(2-(2-aminopyridin-3-yl)-5-phenyl-3H-imidazo[4,5-b]pyridin-3-yl)benzyl)piperazin-1-yl)-5-fluoro-2-hydroxybenzaldehyde;
4-(4-(4-(2-(2-aminopyridin-3-yl)-5-phenyl-3H-imidazo[4,5-b]pyridin-3-yl)benzyl)piperazin-1-yl)-3-fluoro-2-hydroxybenzaldehyde;
4-(4-(4-(2-(2-aminopyridin-3-yl)-5-phenyl-3H-imidazo[4,5-b]pyridin-3-yl)benzyl)piperazin-1-yl)-5-bromo-2-hydroxybenzaldehyde;
4-(4-(4-(2-(2-aminopyridin-3-yl)-5-phenyl-3H-imidazo[4,5-b]pyridin-3-yl)benzyl)piperazin-1-yl)-2-hydroxy-5-vinylbenzaldehyde;
5-(4-(4-(2-(2-aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(3,6-dihydro-2H-pyran-4-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-morpholino-3H-imidazo[4,5-b]pyridin-3-yl)benzyl)piperazin-1-yl)-2-hydroxybenzaldehyde;
5-(4-(4-(2-(2-aminopyridin-3-yl)-5-morpholino-3H-imidazo[4,5-b]pyridin-3-yl)benzyl)piperazine-1-carbonyl)-2-hydroxybenzaldehyde;
4-(4-(4-(2-(2-aminopyridin-3-yl)-5-morpholino-3H-imidazo[4,5-b]pyridin-3-yl)benzyl)piperazine-1-carbonyl)-2-hydroxybenzaldehyde;
3-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile;
3-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile;
2-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile;
2-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile;
4-(2-(2-aminopyridin-3-yl)-3-(4-((4-(3-formyl-4-hydroxybenzoyl)piperazin-1-yl)methyl)phenyl)-3H-imidazo[4,5-b]pyridin-5-yl)benzonitrile;
4-(2-(2-aminopyridin-3-yl)-3-(4-((4-(4-formyl-3-hydroxybenzoyl)piperazin-1-yl)methyl)phenyl)-3H-imidazo[4,5-b]pyridin-5-yl)benzonitrile;
5-(4-(4-(2-(2-aminopyridin-3-yl)-5-(3-fluorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(3-fluorophenyl)-3H-imidazo[4,5-b]pyridin-3-yl)benzyl)piperazine-1-carbonyl)-2-hydroxybenzaldehyde;
5-(4-(4-(2-(2-aminopyridin-3-yl)-5-(3-chlorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(3-chlorophenyl)-3H-imidazo[4,5-b]pyridin-3-yl)benzyl)piperazine-1-carbonyl)-2-hydroxybenzaldehyde;
5-(4-(4-(2-(2-aminopyridin-3-yl)-5-(1-methyl-6-oxo-1,6-dihydropyridin-3-yl)-3H-imidazo[4,5-b]pyridin-3-yl)benzyl)piperazine-1-carbonyl)-2-hydroxybenzaldehyde;
5-(4-(4-(2-(2-aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(6-oxo-1,6-dihydropyridin-2-yl)-3H-imidazo[4,5-b]pyridin-3-yl)benzyl)piperazine-1-carbonyl)-2-hydroxybenzaldehyde;
4-((1-(4-(2-(2-aminopyridin-3-yl)-5-phenyl-3H-imidazo[4,5-b]pyridin-3-yl)benzyl)piperidin-4-yl)amino)-2-hydroxybenzaldehyde;
5-((1-(4-(2-(2-aminopyridin-3-yl)-5-phenyl-3H-imidazo[4,5-b]pyridin-3-yl)benzyl)piperidin-4-yl)amino)-2-hydroxybenzaldehyde;
N-(1-(4-(2-(2-aminopyridin-3-yl)-5-phenyl-3H-imidazo[4,5-b]pyridin-3-yl)benzyl)piperidin-4-yl)-3-formyl-2-hydroxybenzamide;
3-((1-(4-(2-(2-aminopyridin-3-yl)-5-phenyl-3H-imidazo[4,5-b]pyridin-3-yl)benzyl)piperidin-4-yl)amino)-2-hydroxybenzaldehyde;
2-((1-(4-(2-(2-aminopyridin-3-yl)-5-phenyl-3H-imidazo[4,5-b]pyridin-3-yl)benzyl)piperidin-4-yl)amino)-6-hydroxybenzaldehyde;
N-(1-(4-(2-(2-aminopyridin-3-yl)-5-morpholino-3H-imidazo[4,5-b]pyridin-3-yl)benzyl)piperidin-4-yl)-3-formyl-4-hydroxybenzamide;
N-(1-(4-(2-(2-aminopyridin-3-yl)-5-morpholino-3H-imidazo[4,5-b]pyridin-3-yl)benzyl)piperidin-4-yl)-4-formyl-3-hydroxybenzamide;
4-(4-(4-(2-(2-aminopyridin-3-yl)-6-phenyl-3H-imidazo[4,5-b]pyridin-3-yl)benzyl)piperazin-1-yl)-2-hydroxybenzaldehyde;
N-(1-(4-(2-(2-aminopyridin-3-yl)-5-phenyl-3H-imidazo[4,5-b]pyridin-3-yl)benzyl)piperidin-4-yl)-3-formyl-4-hydroxy-N-methylbenzamide;
4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde;
4-((1-(4-(2-(2-aminopyridin-3-yl)-5-phenyl-3H-imidazo[4,5-b]pyridin-3-yl)benzyl)piperidin-4-yl)(methyl)amino)-2-hydroxybenzaldehyde;
(R)—N-(1-(4-(2-(2-aminopyridin-3-yl)-5-phenyl-3H-imidazo[4,5-b]pyridin-3-yl)benzyl)piperidin-3-yl)-3-formyl-4-hydroxybenzamide;
(R)—N-(1-(4-(2-(2-aminopyridin-3-yl)-5-phenyl-3H-imidazo[4,5-b]pyridin-3-yl)benzyl)pyrrolidin-3-yl)-3-formyl-4-hydroxybenzamide;
N-(1-(4-(2-(2-aminopyridin-3-yl)-5-phenyl-3H-imidazo[4,5-b]pyridin-3-yl)benzyl)piperidin-4-yl)-4-formyl-3-hydroxy-N-methylbenzamide;
N-(1-(4-(2-(2-aminopyridin-3-yl)-5-phenyl-3H-imidazo[4,5-b]pyridin-3-yl)benzyl)azetidin-3-yl)-3-formyl-4-hydroxybenzamide;
5-(6-(4-(2-(2-aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde;
(R)—N-(1-(4-(2-(2-aminopyridin-3-yl)-5-phenyl-3H-imidazo[4,5-b]pyridin-3-yl)benzyl)piperidin-3-yl)-4-formyl-3-hydroxybenzamide;
(R)—N-(1-(4-(2-(2-aminopyridin-3-yl)-5-phenyl-3H-imidazo[4,5-b]pyridin-3-yl)benzyl)pyrrolidin-3-yl)-4-formyl-3-hydroxybenzamide;
N-(1-(4-(2-(2-aminopyridin-3-yl)-5-phenyl-3H-imidazo[4,5-b]pyridin-3-yl)benzyl)azetidin-3-yl)-4-formyl-3-hydroxybenzamide;
4-(6-(4-(2-(2-aminopyridin-3-yl)-5-phenyl-3H-imidazo[4,5-b]pyridin-3-yl)benzyl)-2,6-diazaspiro[3.3]heptane-2-carbonyl)-2-hydroxybenzaldehyde;
5-(7-(4-(2-(2-aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde;
N-(7-(4-(2-(2-aminopyridin-3-yl)-5-phenyl-3H-imidazo[4,5-b]pyridin-3-yl)benzyl)-7-azaspiro[3.5]nonan-2-yl)-3-formyl-4-hydroxybenzamide;
N-(2-(4-(2-(2-aminopyridin-3-yl)-5-phenyl-3H-imidazo[4,5-b]pyridin-3-yl)benzyl)-2-azaspiro[3.3]heptan-6-yl)-3-formyl-4-hydroxybenzamide;
N-(2-(4-(2-(2-aminopyridin-3-yl)-5-phenyl-3H-imidazo[4,5-b]pyridin-3-yl)benzyl)-2-azaspiro[3.3]heptan-6-yl)-4-formyl-3-hydroxybenzamide;
4-(7-(4-(2-(2-aminopyridin-3-yl)-5-phenyl-3H-imidazo[4,5-b]pyridin-3-yl)benzyl)-2,7-diazaspiro[3.5]nonane-2-carbonyl)-2-hydroxybenzaldehyde;
4-(4-(4-(2-(2-aminopyridin-3-yl)-5-phenyl-3H-imidazo[4,5-b]pyridin-3-yl)benzyl)-1,4-diazepane-1-carbonyl)-2-hydroxybenzaldehyde;
4-(7-(4-(2-(2-aminopyridin-3-yl)-5-phenyl-3H-imidazo[4,5-b]pyridin-3-yl)benzyl)-2,7-diazaspiro[3.5]nonan-2-yl)-2-hydroxybenzaldehyde;
5-(8-(4-(2-(2-aminopyridin-3-yl)-5-phenyl-3H-imidazo[4,5-b]pyridin-3-yl)benzyl)-2,8-diazaspiro[4.5]decane-2-carbonyl)-2-hydroxybenzaldehyde;
4-(8-(4-(2-(2-aminopyridin-3-yl)-5-phenyl-3H-imidazo[4,5-b]pyridin-3-yl)benzyl)-2,8-diazaspiro[4.5]decane-2-carbonyl)-2-hydroxybenzaldehyde;
4-(8-(4-(2-(2-aminopyridin-3-yl)-5-phenyl-3H-imidazo[4,5-b]pyridin-3-yl)benzyl)-2,8-diazaspiro[4.5]decan-2-yl)-2-hydroxybenzaldehyde;
5-(9-(4-(2-(2-aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde;
4-(9-(4-(2-(2-aminopyridin-3-yl)-5-phenyl-3H-imidazo[4,5-b]pyridin-3-yl)benzyl)-3,9-diazaspiro[5.5]undecan-3-yl)-2-hydroxybenzaldehyde;
4-(6-(4-(2-(2-aminopyridin-3-yl)-5-phenyl-3H-imidazo[4,5-b]pyridin-3-yl)benzyl)-2,6-diazaspiro[3.4]octan-2-yl)-2-hydroxybenzaldehyde;
5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde;
5-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde;
5-(2-(4-(2-(2-aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,8-diazaspiro[4.5]decan-8-yl)-2-hydroxybenzaldehyde;
5-(2-(4-(2-(2-aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,7-diazaspiro[4.5]decane-7-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,7-diazaspiro[4.5]decan-7-yl)-2-hydroxybenzaldehyde;
4-(6-(4-(2-(2-aminopyridin-3-yl)-5-phenyl-3H-imidazo[4,5-b]pyridin-3-yl)benzyl)-2,6-diazaspiro[3.3]heptan-2-yl)-2-hydroxybenzaldehyde;
4-(2-(4-(2-(2-aminopyridin-3-yl)-5-phenyl-3H-imidazo[4,5-b]pyridin-3-yl)benzyl)-2,8-diazaspiro[4.5]decane-8-carbonyl)-2-hydroxybenzaldehyde;
5-(6-(4-(2-(2-aminopyridin-3-yl)-5-phenyl-3H-imidazo[4,5-b]pyridin-3-yl)benzyl)-2,6-diazaspiro[3.3]heptan-2-yl)-2-hydroxybenzaldehyde;
5-(2-(4-(2-(2-aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,6-diazaspiro[3.4]octane-6-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,6-diazaspiro[3.4]octan-6-yl)-2-hydroxybenzaldehyde;
5-(2-(4-(2-(2-aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,7-diazaspiro[3.5]nonane-7-carbonyl)-2-hydroxybenzaldehyde;
4-(2-(4-(2-(2-aminopyridin-3-yl)-5-phenyl-3H-imidazo[4,5-b]pyridin-3-yl)benzyl)-2,7-diazaspiro[3.5]nonan-7-yl)-2-hydroxybenzaldehyde;
(R)-5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
(R)-4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
(R)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde;
(S)-5-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
(S)-4-(7-(4-(2-(2-aminopyridin-3-yl)-5-phenyl-3H-imidazo[4,5-b]pyridin-3-yl)benzyl)-2,7-diazaspiro[4.4]nonane-2-carbonyl)-2-hydroxybenzaldehyde;
(S)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-2-hydroxybenzaldehyde;
(R)-4-(4-(4-(2-(2-aminopyridin-3-yl)-5-phenyl-3H-imidazo[4,5-b]pyridin-3-yl)benzyl)-2-methylpiperazin-1-yl)-2-hydroxybenzaldehyde;
(S)-5-(4-(4-(2-(2-aminopyridin-3-yl)-5-phenyl-3H-imidazo[4,5-b]pyridin-3-yl)benzyl)-2-methylpiperazine-1-carbonyl)-2-hydroxybenzaldehyde;
(S)-4-(4-(4-(2-(2-aminopyridin-3-yl)-5-phenyl-3H-imidazo[4,5-b]pyridin-3-yl)benzyl)-2-methylpiperazine-1-carbonyl)-2-hydroxybenzaldehyde;
(S)-4-(4-(4-(2-(2-aminopyridin-3-yl)-5-phenyl-3H-imidazo[4,5-b]pyridin-3-yl)benzyl)-2-methylpiperazin-1-yl)-2-hydroxybenzaldehyde;
4-(7-(4-(2-(2-aminopyridin-3-yl)-5-phenyl-3H-imidazo[4,5-b]pyridin-3-yl)benzyl)-4,7-diazaspiro[2.5]octan-4-yl)-2-hydroxybenzaldehyde;
4-(3-(4-(2-(2-aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde;
5-(3-(4-(2-(2-aminopyridin-3-yl)-5-phenyl-3H-imidazo[4,5-b]pyridin-3-yl)benzyl)-3,8-diazabicyclo[3.2.1]octane-8-carbonyl)-2-hydroxybenzaldehyde;
4-(3-(4-(2-(2-aminopyridin-3-yl)-5-phenyl-3H-imidazo[4,5-b]pyridin-3-yl)benzyl)-3,8-diazabicyclo[3.2.1]octan-8-yl)-2-hydroxybenzaldehyde;
5-(8-(4-(2-(2-aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde;
4-(8-(4-(2-(2-aminopyridin-3-yl)-5-phenyl-3H-imidazo[4,5-b]pyridin-3-yl)benzyl)-3,8-diazabicyclo[3.2.1]octane-3-carbonyl)-2-hydroxybenzaldehyde;
4-(8-(4-(2-(2-aminopyridin-3-yl)-5-phenyl-3H-imidazo[4,5-b]pyridin-3-yl)benzyl)-3,8-diazabicyclo[3.2.1]octan-3-yl)-2-hydroxybenzaldehyde;
N-(1-(4-(2-(2-aminopyridin-3-yl)-5-phenyl-3H-imidazo[4,5-b]pyridin-3-yl)benzyl)azepan-4-yl)-3-formyl-4-hydroxybenzamide;
N-(1-(4-(2-(2-aminopyridin-3-yl)-5-phenyl-3H-imidazo[4,5-b]pyridin-3-yl)benzyl)azepan-4-yl)-4-formyl-3-hydroxybenzamide;
N-(1-(4-(2-(2-aminopyridin-3-yl)-5-phenyl-3H-imidazo[4,5-b]pyridin-3-yl)benzyl)azepan-3-yl)-3-formyl-4-hydroxybenzamide;
N-(1-(4-(2-(2-aminopyridin-3-yl)-5-phenyl-3H-imidazo[4,5-b]pyridin-3-yl)benzyl)azepan-3-yl)-4-formyl-3-hydroxybenzamide;
5-(4-(4-(2-(2-aminopyridin-3-yl)-5-phenyl-3H-imidazo[4,5-b]pyridin-3-yl)benzyl)-1,4-diazepane-1-carbonyl)-2-hydroxybenzaldehyde;
4-(4-(4-(2-(2-aminopyridin-3-yl)-5-phenyl-3H-imidazo[4,5-b]pyridin-3-yl)benzyl)-1,4-diazepan-1-yl)-2-hydroxybenzaldehyde;
5-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde;
4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carbonyl)-2-hydroxybenzaldehyde;
4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde;
4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-2-hydroxybenzaldehyde;
4-(5-(4-(2-(2-aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde;
5-(5-(4-(2-(2-aminopyridin-3-yl)-5-phenyl-3H-imidazo[4,5-b]pyridin-3-yl)benzyl)-2,5-diazabicyclo[2.2.2]octane-2-carbonyl)-2-hydroxybenzaldehyde;
4-(5-(4-(2-(2-aminopyridin-3-yl)-5-phenyl-3H-imidazo[4,5-b]pyridin-3-yl)benzyl)-2,5-diazabicyclo[2.2.2]octan-2-yl)-2-hydroxybenzaldehyde;
2-((1-(4-(2-(2-aminopyridin-3-yl)-5-phenyl-3H-imidazo[4,5-b]pyridin-3-yl)benzyl)piperidin-4-yl)oxy)-6-hydroxybenzaldehyde;
4-((1-(4-(2-(2-aminopyridin-3-yl)-5-phenyl-3H-imidazo[4,5-b]pyridin-3-yl)benzyl)piperidin-4-yl)oxy)-2-hydroxybenzaldehyde;
5-((1-(4-(2-(2-aminopyridin-3-yl)-5-phenyl-3H-imidazo[4,5-b]pyridin-3-yl)benzyl)piperidin-4-yl)oxy)-2-hydroxybenzaldehyde;
5-((3aR,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde;
4-((3aR,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde;
4-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde;
5-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-2-hydroxybenzaldehyde;
5-((3aS,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde;
4-((3aS,6aS)-5-(4-(2-(2-aminopyridin-3-yl)-5-phenyl-3H-imidazo[4,5-b]pyridin-3-yl)benzyl)octahydropyrrolo[3,4-c]pyrrole-2-carbonyl)-2-hydroxybenzaldehyde;
2-((4-(2-(2-aminopyridin-3-yl)-5-phenyl-3H-imidazo[4,5-b]pyridin-3-yl)benzyl)oxy)-6-hydroxybenzaldehyde;
4-((4-(2-(2-aminopyridin-3-yl)-5-phenyl-3H-imidazo[4,5-b]pyridin-3-yl)benzyl)oxy)-2-hydroxybenzaldehyde;
5-((4-(2-(2-aminopyridin-3-yl)-5-phenyl-3H-imidazo[4,5-b]pyridin-3-yl)benzyl)oxy)-2-hydroxybenzaldehyde;
(S)—N-(1-(4-(2-(2-aminopyridin-3-yl)-5-phenyl-3H-imidazo[4,5-b]pyridin-3-yl)phenyl)ethyl)-3-formyl-4-hydroxybenzamide;
5-(4-(4-(2-(2-aminopyridin-3-yl)-5-(2-fluorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(2-fluorophenyl)-3H-imidazo[4,5-b]pyridin-3-yl)benzyl)piperazine-1-carbonyl)-2-hydroxybenzaldehyde;
5-(4-(4-(2-(2-aminopyridin-3-yl)-5-(4-fluorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(4-fluorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(2-chlorophenyl)-3H-imidazo[4,5-b]pyridin-3-yl)benzyl)piperazin-1-yl)-2-hydroxybenzaldehyde;
4-(4-(4-(2-(2-aminopyridin-3-yl)-5-(4-chlorophenyl)-3H-imidazo[4,5-b]pyridin-3-yl)benzyl)piperazin-1-yl)-2-hydroxybenzaldehyde;
5-(4-(4-(2-(2-aminopyridin-3-yl)-5-(4-chlorophenyl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(4-chlorophenyl)-3H-imidazo[4,5-b]pyridin-3-yl)benzyl)piperazine-1-carbonyl)-2-hydroxybenzaldehyde;
5-(4-(4-(2-(2-aminopyridin-3-yl)-5-(4-chlorophenyl)-3H-imidazo[4,5-b]pyridin-3-yl)benzyl)piperazin-1-yl)-2-hydroxybenzaldehyde;
5-(4-(4-(2-(2-aminopyridin-3-yl)-5-(cyclohex-1-en-1-yl)-3H-imidazo[4,5-b]pyridin-3-yl)benzyl)piperazine-1-carbonyl)-2-hydroxybenzaldehyde;
4-(4-(4-(2-(2-aminopyridin-3-yl)-5-(cyclohex-1-en-1-yl)-3H-imidazo[4,5-b]pyridin-3-yl)benzyl)piperazine-1-carbonyl)-2-hydroxybenzaldehyde;
4-(9-(4-(2-(2-aminopyridin-3-yl)-5-phenyl-3H-imidazo[4,5-b]pyridin-3-yl)benzyl)-3,9-diazaspiro[5.5]undecane-3-carbonyl)-2-hydroxybenzaldehyde; and
4-(6-(4-(2-(2-aminopyridin-3-yl)-5-phenyl-3H-imidazo[4,5-b]pyridin-3-yl)benzyl)-2,6-diazaspiro[3.4]octane-2-carbonyl)-2-hydroxybenzaldehyde.

74. A pharmaceutical composition comprising a compound, or pharmaceutically acceptable salt or solvate thereof, as described in claim 54, and a pharmaceutically acceptable excipient.

75. A method of treating cancer in a patient in need thereof, comprising administering to the patient a compound as described in claim 54, or pharmaceutically acceptable salt or solvate thereof.
